(19) United States
(12) Patent Application Publication
(10) Pub. No.: US 2011/0028447 A1
(43) Pub. Date: Feb. 3, 2011

(54) INDAZOLE DERIVATIVES

(75) Inventors: Ingrid Price Buchler, Cambridge, MA (US); Michael Joseph Hayes, Saint Charles, MO (US); Shridhar Gajanan Hegde, Germantown, TN (US); Susan Landis Hockerman, Kirkwood, MO (US); Darin Eugene Jones, Baldwin, MO (US); Steven Wade Kortum, Groton, CT (US); Joseph Gerace Rico, Kalamazoo, MI (US); Ruth Elizabeth TenBrink, Labadie, MO (US); Kun Ken Wu, Chesterfield, MO (US)

Correspondence Address: PFIZER INC.;PATENT DEPARTMENT, Bld 114 M/S 9114, EASTERN POINT ROAD, GROTON, CT, 06340, US

(73) Assignee: Pfizer Inc.

(21) Appl. No.: 12/918,914

(22) PCT Filed: Feb. 26, 2009
(86) PCT No.: PCT/IB2009/000432

§ 371 Date: Aug. 23, 2010

Related U.S. Application Data
(60) Provisional application No. 61/032,657, filed on Feb. 29, 2008.

(51) Int. Cl. A61K 31/541; C07D 231/56; C07D 413/06; C07D 401/06; C07D 401/10; C07D 413/12; C07D 471/04; C07D 413/10; C07D 405/12; C07D 403/12; C07D 401/12; C07D 417/12; A61P 29/00; A61K 31/416; A61K 31/422; A61K 31/4439; A61K 31/4245; A61K 31/437; A61K 31/41; A61K 31/5377; A61K 31/454; A61K 31/496; A61K 31/4178; A61K 31/513; A61K 31/4196; A61K 31/4192; A61K 31/427; A61K 31/433

(52) U.S. Cl. 514/210.18; 548/362.5; 548/247; 546/275.7; 548/143; 546/120; 548/236; 548/253; 548/131; 544/140; 544/371; 544/58.2; 548/311.7; 548/229; 544/319; 548/266.4; 548/262.4; 548/255; 548/214; 548/139; 514/406; 514/378; 514/338; 514/364; 514/303; 514/374; 514/381; 514/234.5; 514/322; 514/254.06; 514/228.2; 514/402; 514/376; 514/269; 514/383; 514/372; 514/363

(57) ABSTRACT

This invention relates to compounds, pharmaceutical compositions and methods for the treatment of a condition mediated by CB1 receptor activity in a mammalian subject including a human, which comprises administering to a mammal in need of such treatment a therapeutically effective amount of the compound of formula (I) wherein R¹, R² and R³ are as defined in this specification.

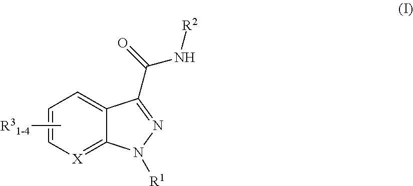

### FIELD OF THE INVENTION

The present invention provides pharmaceutically active indazole compounds and analogues. Such compounds have cannabinoid (CB)1 receptor binding activity. The present invention also relates to pharmaceutical compositions, methods of treatment and use, comprising the above derivatives for the treatment of disease conditions mediated by CB1 receptor binding activity.

### BACKGROUND OF THE INVENTION

Cannabinoid receptors, endogenous cannabinoids and the enzymes that synthesize and degrade endocannabinoids make up the endocannabinoid system. CB1 and CB2 are two subtypes of cannabinoid receptors. CB1 and CB2 are both G protein coupled receptors. CB1 receptors primarily exist in the central nervous system, but are also found in some peripheral tissues including pituitary gland, immune cells, reproductive tissues, gastrointestinal tissues, sympathetic ganglia, heart, lung, urinary bladder and adrenal gland. CB2 receptors primarily exist in immune cells. Cannabinoid agonists are believed to be useful in the treatment of pain and several other indications.

There is a need to provide new CB1 ligands that are good drug candidates. They should be well absorbed from the gastrointestinal tract, be metabolically stable and possess favorable pharmacokinetic properties. Furthermore, the ideal drug candidate will exist in a physical form that is stable, non-hygroscopic and easily formulated.

### SUMMARY OF THE INVENTION

The present invention is directed to pharmaceutically active indazole compounds.

Such compounds are useful for as CB1 agonists.

This invention is directed, in part, to compounds that generally fall within the structure of Formula I:

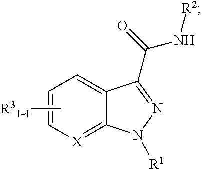

or a pharmaceutically acceptable salt thereof, wherein

X is CH or N;

R¹ is
R⁴₁₋₅-aryl-(CH₂)ₙ— or
R⁵₁₋₅-heteroaryl-(CH₂)ₙ—; wherein
   each R⁴ is independently H, halo, cyano, NH₂—C(O)—, C₁-C₆ alkoxy-, trifluoromethyl or C₁-C₆ alkoxy-C(O)—;
   each R⁵ is independently H or C₁-C₆ alkyl;

R² is
NR¹¹R¹²—C(O)—R¹³CH—,
R¹⁴—C(O)—NR¹⁵—(CH₂)ₙ—R¹³CH—,
R¹⁶—C(O)—R¹³CH—,
C₁-C₆ alkoxy-C(O)—(CH₂), —NR¹⁵—C(O)—R¹³CH—,
NR¹⁷R¹⁸—C(O)—(CH₂)ₙ—NR¹⁹—C(O)—R¹³CH—,
R²⁰—SO₂—NR²¹—(CH₂)ₙ—R¹³CH—,
R²²R²³CH—,
R²⁴₁₋₅-heteroaryl,
R²⁴₁₋₅-heteroaryl-R¹³CH—,
R²⁴₁₋₅-heteroaryl-NR¹⁵—C(O)—R¹³CH—,
R²⁵₁₋₅-heterocyclyl,
R²⁵₁₋₅-heterocyclyl-(CH₂)ₙ—,
R²⁶₁₋₅—C₃-C₇ cycloalkyl,
NR²⁷R²⁸—(CH₂)ₙ—NR²⁹—C(O)—R¹³CH—,
R³⁰—SO₂—NR³¹—(CH₂)ₙ—NR¹⁵—C(O)—R¹³CH—,
R³⁰—SO₂—(CH₂)ₙ—NR³¹—C(O)—R¹³CH—,
R³²—C(O)—R³³CH—NR³⁴—C(O)—R¹³CH—,
R³²—C(O)—(CH₂)ₙ—NR³⁴—C(O)—R¹³CH—,
R³⁵₁₋₅-heteroaryl-(CH₂)ₙ—NR³⁶—C(O)—R¹³CH—,
R³⁷₁₋₅-heterocyclyl-(CH₂)ₙ—NR³⁶—C(O)—R¹³CH—,
R³⁷₁₋₅-heterocyclyl-C(O)—R¹³CH—,
R³⁸₁₋₅-aryl-R³⁹C—NR⁴⁰—C(O)—R¹³CH—,
R³⁸₁₋₅-aryl-(CH₂)ₙ—NR⁴⁰—C(O)—R¹³CH—,
R⁴¹₁₋₅-aryl-(CH₂)ₙ—,
NR¹⁷R¹⁸—C(O)—CH(R⁴²)—NR¹⁹—C(O)—R¹³CH—, or
R⁴³—CH(OH)—CH₂—NR¹⁹—C(O)—R¹³CH—;
wherein
   R¹¹ and R¹² are independently H, OH, C₁-C₆ alkyl, C₁-C₆ haloalkyl, OH—C₁-C₆ alkyl, (OH)₂—C₁-C₆ alkyl, (OH)₃—C₄-C₆ alkyl, C₁-C₆ alkoxy-(CH₂)ₙ—, C₃-C₇ cycloalkyl, benzo-fused C₃-C₇ cycloalkyl, cyano-C₁-C₆ alkyl, NH₂—C(NH)—C₁-C₆ alkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl-, OH—C₃-C₇ cycloalkyl-, C₁-C₆ alkoxy-C(O)—C₃-C₇ cycloalkyl-, (C₁-C₆ alkoxy-aryl)-C₃-C₇ cycloalkyl-, NH₂—C(O)—C₃-C₇ cycloalkyl-, OH-aryl, or R²⁴₁₋₅-heteroaryl-O—(CH₂)ₙ—;
   R¹³ is H, C₁-C₆ alkyl, OH—C₁-C₆ alkyl, aryl, aryl-(CH₂)ₙ—, or C₃-C₇ cycloalkyl;
   R¹⁴ is (C₁-C₆ alkyl)₂N—, aryl, C₁-C₆ alkyl, or C₃-C₇ cycloalkyl;
   R¹⁵, R²¹, R²⁹, R³¹, R³⁴, and R⁴⁰ are independently H or C₁-C₆ alkyl;
   R¹⁶ is OH or C₁-C₆ alkoxy;
   R¹⁷ and R¹⁸ are independently H, C₁-C₆ alkyl, C₃-C₇ cycloalkyl, OH—C₁-C₆ alkyl, (OH)₂—C₁-C₆ alkyl, or R²⁴₁₋₅-heteroaryl-;
   each R¹⁹ is independently H or C₁-C₆ alkyl;
   R²⁰ is C₁-C₆ alkyl, C₁-C₆ haloalkyl, or (C₁-C₆ alkyl)₂N—;
   R²² and R²³ are independently C₁-C₆ alkyl, C₃-C₇ cycloalkyl-(CH₂)ₙ—, OH—C₁-C₆ alkyl, aryl, or aryl-OH—C₁-C₆ alkylene;
   each R²⁴ is independently H, C₁-C₆ alkyl, C₃-C₇ cycloalkyl, C₁-C₆ haloalkyl, oxo, OH, NH₂, C₁-C₆ alkoxy-C(O)—, NH₂—C(O)—(CH₂)ₙ—, NH₂—C(O)—, NH₂—C(O)—NH—, OH—C(O)—, NH₂—C(O)—(CH₂)ₙ—NH—C(O)—, (OH)₂—C₁-C₆ alkyl-NH—C(O)—, OH—C₁-C₆ alkyl-NH—C(O)—, or C₃-C₇ cycloalkyl-C(O)—NH—;
   each R²⁵ is independently H or oxo;
   each R²⁶ is independently H, OH, OH—C₁-C₆ alkyl, aryl-(CH₂)ₙ—O—, NH₂—C(O)— or C₁-C₆ alkoxy-C(O)—;
   R²⁷ and R²⁸ independently are H, NH₂—C(O)—, C₃-C₇ cycloalkyl-C(O)—, or R²⁴₁₋₅-heteroaryl-;
   R³⁰ is C₁-C₆ alkyl, C₃-C₇ cycloalkyl, NH₂, C₁-C₆ alkyl-NH—, C₃-C₇ cycloalkyl-(CH₂)ₙNH—, morpholin-4-yl, or R³⁸₁₋₅-phenyl;
   R³² is OH or C₁-C₆ alkoxy-;
   each R³³ is independently H, C₁-C₆ alkyl, or OH—C₁-C₆ alkyl;
   each R³⁵ is independently H, C₁-C₆ alkyl, NH₂—C(O)—, C₁-C₆ alkoxy-C(O)—, C₃-C₇ cycloalkyl, OH, phenyl, or heteroaryl, or two adjacent R³⁵ groups may together form —(CH₂)₃₋₆—;
   each R³⁶ is independently H, C₁-C₆ alkyl, C₁-C₆ alkoxy-, or NH₂—C(O)—;
   each R³⁷ is independently H, NH₂C(O)—, OH, halo, cyano, oxo, OH—C₁-C₆ alkyl, (OH)₂—C₁-C₆ alkyl, NH₂C(O)—(CH₂)ₙ—, NH₂C(O)—(CH₂)ₙ—C(O)—, NH₂C(O)—NH—(CH₂)ₙ—, C₁-C₆ alkyl-NH—C(O)—O—, (OH)—C₁-C₆ alkyl-NH—C(O)—, (OH)₂—C₁-C₆ alkyl-NH—C(O)—, C₁-C₆ alkyl-C(O)—, C₁-C₆ alkoxy-C(O)—, C₃-C₇ cycloalkyl-C(O)—NH—(CH₂)ₙ—, C₁-C₆ alkyl-SO₂—, C₃-C₇ cycloalkyl-SO₂—, or C₃-C₇ cycloalkyl-SO₂—NH—(CH₂)ₙ—;
   each R³⁸ is independently H, NH₂SO₂—, cyano, heteroaryl, OH, halo, C₁-C₆ alkoxy, OH—C(O)—, or C₁-C₆ alkoxy-C(O)—;
   each R³⁹ is independently H, C₁-C₆ alkyl, or OH—C₁-C₆ alkyl;
   each R⁴¹ is independently H, C₁-C₆ alkoxy or halo;
   R⁴² is H, C₁-C₆ alkyl, OH—C₁-C₆ alkyl, aryl, aryl-(CH₂)ₙ— or NH₂—C(O)—CH₂;
   R⁴³ is OH—C(O)—, C₁-C₆ alkoxy-C(O)—, NH₂—C(O)— or R⁴⁴R⁴⁵NCH₂—; and
   R⁴⁴ and R⁴⁵ are independently C₁-C₆ alkyl or OH—C₁-C₆ alkyl, or
   R⁴⁴ and R⁴⁵ together with the nitrogen atom to which they are attached form a pyrrolidine, piperidine or morpholine ring;

n is an integer from 1 to 6; and

each R³ is independently H, halo, C₁-C₆ alkyl, aryl, NH₂—C(O)—, C₁-C₆ alkoxy or heteroaryl.

This invention also includes pharmaceutically acceptable salts, solvates and hydrates. This invention also includes all tautomers and stereochemical isomers of these compounds.

This invention also is directed, in part, to a method for treating a CB1 mediated disorder in a mammal. Such CB1 mediated disorders include pain, rheumatoid arthritis and osteoarthritis. The method comprises administering an above-described compound or pharmaceutically acceptable salt thereof, to the mammal in an amount that is therapeutically-effective to treat the condition.

Further benefits of Applicants' invention will be apparent to one skilled in the art from reading this specification.

### DETAILED DESCRIPTION OF THE INVENTION

The invention will be more carefully understood from the following description given by way of example only. The present invention is directed to a class of indazole compounds. In particular, the present invention is directed to indazole compounds useful as CB1 agonists. While the present invention is not so limited, an appreciation of various aspects of the invention will be gained through the following discussion and the examples provided below.

### DEFINITIONS

The following is a list of definitions of various terms used herein:

The symbol [symbol] represents the point of attachment.

The term “alkane” refers to a saturated acyclic hydrocarbon which can be either a straight chain or branched chain.

The term “alkyl” refers to a straight or branched chain univalent radical derived from an alkane by removal of one hydrogen. Examples of such alkyl radicals are methyl, ethyl, n-propyl, isopropyl, n-butyl, isobutyl, sec-butyl, t-butyl, pentyl, neopentyl, hexyl, isohexyl, and the like.

The term “alkylene” refers to a straight chain or branched bivalent radical derived from alkane by the removal of H from each of the two terminal carbons. Examples include methylene:

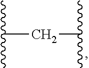

ethylene:

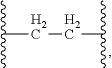

propylene:

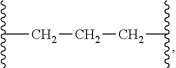

isopropylene:

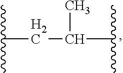

and the like.

The term “alkoxy” means alkyl-O—, wherein alkyl is as defined above. Examples of such a substituent include methoxy (CH₃—O—), ethoxy, n-propoxy, isopropoxy, n-butoxy, iso-butoxy, sec-butoxy, and tert-butoxy.

The term “cycloalkyl” means a saturated carbocyclyl substituent containing from 3 to about 20 carbon atoms. A cycloalkyl may be a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “aryl” means an aromatic carbocyclyl containing from 6 to 14 carbon ring atoms. The term aryl embraces both single and multiple rings. Examples of aryls include phenyl, naphthalenyl, and indenyl.

The term “arylalkyl” means alkyl substituted with aryl, wherein alkyl and aryl are as defined above.

The term “carboxy” or “carboxyl” means OH—C(O)—, which also may be depicted as:

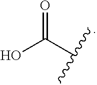

The term “formyl” means HC(O)—, which may also be depicted as:

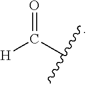

The symbol “C(O)” means C═O which also may be depicted as:

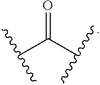

The term “oxo” means a keto radical, and may be depicted as ═O.

The term “hydroxy” or “hydroxyl” means OH—.

The term “hydroxyalkyl” means alkyl substituted with one more hydroxyl, wherein hydroxyl and alkyl are as defined above.

The term “halo” or “halogen” refers to bromo, chloro, fluoro or iodo.

The term “oxy” means an ether substituent, and may be depicted as —O—.

The term “sulfonyl” means SO₂—.

The term “thio” means SH—. The term “alkylthio” is an alkyl substituted thio, which is also depicted as:

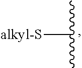

wherein thio and alkyl are as defined above.

The term “heterocyclyl” means a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of heterocyclyls include piperidinyl, morpholinyl, thiomorpholinyl, tetrahydrofuranyl, tetrahydropyranyl, pyrrolidinyl, piperazinyl and diazepanyl.

The term “heteroaryl” means an aromatic heterocyclyl containing from 5 to 14 ring atoms. A heteroaryl may be a single ring or 2 or 3 fused rings. Examples of heteroaryl substituents include isoxazolyl, pyridinyl, furyl, oxadiazolyl, tetrazolyl, dihydroimidazolyl, thiadiazolyl, oxazolyl, triazolyl and dihydroisoxazolyl.

The terms “substituent” and “radical” are interchangeable. If substituents are described as being “independently selected” from a group, each substituent is selected independent of the other. Each substituent therefore may be identical to or different from the other substituent(s).

The term “pharmaceutically-acceptable” is used adjectivally in this specification to mean that the modified noun is appropriate for use as a pharmaceutical product or as a part of a pharmaceutical product.

### Compounds of the Invention

In a first embodiment, this invention is directed to compounds of Formula I:

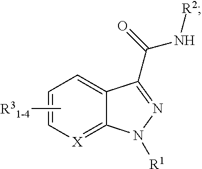

or a pharmaceutically acceptable salt thereof, wherein

X is CH or N;

R¹ is
R⁴₁₋₅-aryl-(CH₂)ₙ— or
R⁵₁₋₅-heteroaryl-(CH₂)ₙ—; wherein
   each R⁴ is independently H, halo, cyano, NH₂—C(O)—, C₁-C₆ alkoxy-, trifluoromethyl or C₁-C₆ alkoxy-C(O)—;
   each R⁵ is independently H or C₁-C₆ alkyl;

R² is
NR¹¹R¹²—C(O)—R¹³CH—,
R¹⁴—C(O)—NR¹⁵—(CH₂)ₙ—R¹³CH—,
R¹⁶—C(O)—R¹³CH—, C₁-C₆ alkoxy-C(O)—(CH₂)ₙ—NR¹⁵—C(O)—R¹³CH—,
NR¹⁷R¹⁸—C(O)—(CH₂)ₙ—NR¹⁹—C(O)—R¹³CH—,
R²⁰—SO₂—NR²¹—(CH₂)ₙ—R¹³CH—,
R²²R²³CH—,
R²⁴₁₋₅-heteroaryl,
R²⁴₁₋₅-heteroaryl-R¹³CH—,
R²⁴₁₋₅-heteroaryl-NR¹⁵—C(O)—R¹³CH—,
R²⁵₁₋₅-heterocyclyl,
R²⁵₁₋₅-heterocyclyl-(CH₂)ₙ—,
R²⁶₁₋₅—C₃-C₇ cycloalkyl,
NR²⁷R²⁸—(CH₂)ₙ—NR²⁹—C(O)—R¹³CH—,
R³⁰—SO₂—NR³¹—(CH₂)ₙ—NR¹⁵—C(O)—R¹³CH—,
R³⁰—SO₂—(CH₂)ₙ—NR³¹—C(O)—R¹³CH—,
R³²—C(O)—R³³CH—NR³⁴—C(O)—R¹³CH—,
R³²—C(O)—(CH₂)ₙ—NR³⁴—C(O)—R¹³CH—,
R³⁵₁₋₅-heteroaryl-(CH₂)ₙ—NR³⁶—C(O)—R¹³CH—,
R³⁷₁₋₅-heterocyclyl-(CH₂)ₙ—NR³⁶—C(O)—R¹³CH—,
R³⁷₁₋₅-heterocyclyl-C(O)—R¹³CH—,
R³⁸₁₋₅-aryl-R³⁹C—NR⁴⁰—C(O)—R¹³CH—,
R³⁸₁₋₅-aryl-(CH₂)ₙ—NR⁴⁰—C(O)—R¹³CH—,
R⁴¹₁₋₅-aryl-(CH₂)ₙ—,
NR¹⁷R¹⁸—C(O)—CH(R⁴²)—NR¹⁹—C(O)—R¹³CH—, or
R⁴³—CH(OH)—CH₂—NR¹⁹—C(O)—R¹³CH—;
wherein
   R¹¹ and R¹² are independently H, OH, C₁-C₆ alkyl, C₁-C₆ haloalkyl, OH—C₁-C₆ alkyl, (OH)₂—C₁-C₆ alkyl, (OH)₃—C₄-C₆ alkyl, C₁-C₆ alkoxy-(CH₂)ₙ—, C₃-C₇ cycloalkyl, benzo-fused C₃-C₇ cycloalkyl, cyano-C₁-C₆ alkyl, NH₂—C(NH)—C₁-C₆ alkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl-, OH—C₃-C₇ cycloalkyl-, C₁-C₆ alkoxy-C(O)—C₃-C₇ cycloalkyl-, (C₁-C₆ alkoxy-aryl)-C₃-C₇ cycloalkyl-, NH₂—C(O)—C₃-C₇ cycloalkyl-, OH-aryl, or R²⁴₁₋₅-heteroaryl-O—(CH₂)ₙ—;
   R¹³ is H, C₁-C₆ alkyl, OH—C₁-C₆ alkyl, aryl, aryl-(CH₂)ₙ—, or C₃-C₇ cycloalkyl;
   R¹⁴ is (C₁-C₆ alkyl)₂N—, aryl, C₁-C₆ alkyl, or C₃-C₇ cycloalkyl;
   R¹⁵, R²¹, R²⁹, R³¹, R³⁴, and R⁴⁰ are independently H or C₁-C₆ alkyl;
   R¹⁶ is OH or C₁-C₆ alkoxy;
   R¹⁷ and R¹⁸ are independently H, C₁-C₆ alkyl, C₃-C₇ cycloalkyl, OH—C₁-C₆ alkyl, (OH)₂—C₁-C₆ alkyl, or R²⁴₁₋₅-heteroaryl-;
   each R¹⁹ is independently H or C₁-C₆ alkyl;
   R²⁰ is C₁-C₆ alkyl, C₁-C₆ haloalkyl, or (C₁-C₆ alkyl)₂N—;
   R²² and R²³ are independently C₁-C₆ alkyl, C₃-C₇ cycloalkyl-(CH₂)ₙ—, OH—C₁-C₆ alkyl, aryl, or aryl-OH—C₁-C₆ alkylene;
   each R²⁴ is independently H, C₁-C₆ alkyl, C₃-C₇ cycloalkyl, C₁-C₆ haloalkyl, oxo, OH, NH₂, C₁-C₆ alkoxy-C(O)—, NH₂—C(O)—(CH₂)ₙ—, NH₂—C(O)—, NH₂—C(O)—NH—, OH—C(O)—, NH₂—C(O)—(CH₂)ₙ—NH—C(O)—, (OH)₂—C₁-C₆ alkyl-NH—C(O)—, OH—C₁-C₆ alkyl-NH—C(O)—, or C₃-C₇ cycloalkyl-C(O)—NH—;
   each R²⁵ is independently H or oxo;
   each R²⁶ is independently H, OH, OH—C₁-C₆ alkyl, aryl-(CH₂)ₙ—O—, NH₂—C(O)— or C₁-C₆ alkoxy-C(O)—;
   R²⁷ and R²⁸ independently are H, NH₂—C(O)—, C₃-C₇ cycloalkyl-C(O)—, or R²⁴₁₋₅-heteroaryl-;
   R³⁰ is C₁-C₆ alkyl, C₃-C₇ cycloalkyl, NH₂, C₁-C₆ alkyl-NH—, C₃-C₇ cycloalkyl-(CH₂)ₙ—NH—, morpholin-4-yl, or R³⁸₁₋₅-phenyl;
   R³² is OH or C₁-C₆ alkoxy-;
   each R³³ is independently H, C₁-C₆ alkyl, or OH—C₁-C₆ alkyl;
   each R³⁵ is independently H, C₁-C₆ alkyl, NH₂—C(O)—, C₁-C₆ alkoxy-C(O)—, C₃-C₇ cycloalkyl, OH, phenyl, or heteroaryl, or two adjacent R³⁵ groups may together form —(CH₂)₃₋₆—;
   each R³⁶ is independently H, C₁-C₆ alkyl, C₁-C₆ alkoxy-, or NH₂—C(O)—;
   each R³⁷ is independently H, NH₂C(O)—, OH, halo, cyano, oxo, OH—C₁-C₆ alkyl, (OH)₂—C₁-C₆ alkyl, NH₂C(O)—(CH₂)ₙ—, NH₂C(O)—(CH₂)ₙ—C(O)—, NH₂C(O)—NH—(CH₂)ₙ—, C₁-C₆ alkyl-NH—C(O)—O—, (OH)—C₁-C₆ alkyl-NH—C(O)—, (OH)₂—C₁-C₆ alkyl-NH—C(O)—, C₁-C₆ alkyl-C(O)—, C₁-C₆ alkoxy-C(O)—, C₃-C₇ cycloalkyl-C(O)—NH—(CH₂)ₙ—, C₁-C₆ alkyl-SO₂—, C₃-C₇ cycloalkyl-SO₂—, or C₃-C₇ cycloalkyl-SO₂—NH—(CH₂)ₙ—;
   each R³⁸ is independently H, NH₂SO₂—, cyano, heteroaryl, OH, halo, C₁-C₆ alkoxy, OH—C(O)—, or C₁-C₆ alkoxy-C(O)—;
   each R³⁹ is independently H, C₁-C₆ alkyl, or OH—C₁-C₆ alkyl;
   each R⁴¹ is independently H, C₁-C₆ alkoxy or halo;
   R⁴² is H, C₁-C₆ alkyl, OH—C₁-C₆ alkyl, aryl, aryl-(CH₂)ₙ— or NH₂—C(O)—CH₂;
   R⁴³ is OH—C(O)—, C₁-C₆ alkoxy-C(O)—, NH₂—C(O)— or R⁴⁴R⁴⁵NCH₂—; and
   R⁴⁴ and R⁴⁵ are independently C₁-C₆ alkyl or OH—C₁-C₆ alkyl, or
   R⁴⁴ and R⁴⁵ together with the nitrogen atom to which they are attached form a pyrrolidine, piperidine or morpholine ring;
n is an integer from 1 to 6; and
each R³ is independently H, halo, C₁-C₆ alkyl, aryl, NH₂—C(O)—, C₁-C₆ alkoxy or heteroaryl.

Among its many further embodiments, the present invention includes compounds or pharmaceutically acceptable salts thereof, having a structure according to Formula I:

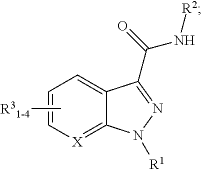

wherein

X is CH or N;

R¹ is R⁴₁₋₅-aryl-(CH₂)ₙ— or R⁵₁₋₅-heteroaryl-(CH₂)ₙ—; wherein
each R⁴ is independently H, halo, cyano or NH₂—C(O)—;
each R⁵ is independently H or C₁-C₆ alkyl;

R² is NR¹¹R¹²—C(O)—R¹³CH—, R¹⁴—C(O)—NR¹⁵—(CH₂)ₙ—R¹³CH—, R¹⁶—C(O)—R¹³CH—, C₁-C₆ alkoxy-C(O)—(CH₂)ₙ—NR¹⁵—C(O)—R¹³CH—, NR¹⁷R¹⁸—C(O)—(CH₂)ₙ—NR¹⁹—C(O)—R¹³CH—, R²⁰—SO₂—NR²¹—(CH₂)ₙ—R¹³CH—, R²²R²³CH—, R²⁴₁₋₅-heteroaryl, R²⁴₁₋₅-heteroaryl-R¹³CH—, R²⁴₁₋₅-heteroaryl-NR¹⁵—C(O)—R¹³CH—, R²⁵₁₋₅-heterocyclyl, R²⁵₁₋₅-heterocyclyl-(CH₂)ₙ—, R²⁶₁₋₅—C₃-C₇ cycloalkyl, NR²⁷R²⁸—(CH₂)ₙ—NR²⁹—C(O)—R¹³CH—, R³⁰—SO₂—NR³¹—(CH₂)ₙ—NR¹⁵—C(O)—R¹³CH—, R³⁰—SO₂—(CH₂)ₙ—NR³¹—C(O)—R¹³CH—, R³²—C(O)—R³³CH—NR³⁴—C(O)—R¹³CH—, R³²—C(O)—(CH₂)ₙ—NR³⁴—C(O)—R¹³CH—, R³⁵₁₋₅-heteroaryl-(CH₂)ₙ—NR³⁶—C(O)—R¹³CH—, R³⁷₁₋₅-heterocyclyl-(CH₂)ₙ—NR³⁶—C(O)—R¹³CH—, R³⁷₁₋₅-heterocyclyl-C(O)—R¹³CH—, R³⁸₁₋₅-aryl-R³⁹C—NR⁴⁰—C(O)—R¹³CH—, R³⁸₁₋₅-aryl-(CH₂)ₙ—NR⁴⁰—C(O)—R¹³CH— or R⁴¹₁₋₅-aryl-(CH₂)ₙ—; wherein
R¹¹ and R¹² are independently H, C₁-C₆ alkyl, OH—C₁-C₆ alkyl, (OH)₂—C₁-C₆ alkyl, C₁-C₆ alkoxy-(CH₂)ₙ—, C₃-C₇ cycloalkyl, cyano-C₁-C₆ alkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl-, or OH-aryl;
R¹³ is H, C₁-C₆ alkyl, OH—C₁-C₆ alkyl, aryl, aryl-(CH₂)ₙ—, or C₃-C₇ cycloalkyl;
R¹⁴ is (C₁-C₆ alkyl)₂N—, aryl, C₁-C₆ alkyl, or C₃-C₇ cycloalkyl;
R¹⁵, R²¹, R²⁹, R³¹, R³³, R³⁴, R³⁶, R³⁹ and R⁴⁰ are independently H or C₁-C₆ alkyl;
R¹⁶ is OH or C₁-C₆ alkoxy;
R¹⁷, R¹⁸ and R¹⁹ are independently H or C₁-C₆ alkyl;
R²⁰ is C₁-C₆ alkyl, C₁-C₆ haloalkyl, or (C₁-C₆ alkyl)₂N—;
R²² and R²³ are independently C₁-C₆ alkyl, C₃-C₇ cycloalkyl-(CH₂)ₙ—, OH—C₁-C₆ alkyl, aryl, or aryl-OH—C₁-C₆ alkylene;
each R²⁴ is independently H, C₁-C₆ alkyl, C₃-C₇ cycloalkyl, C₁-C₆ haloalkyl, oxo, NH₂, C₁-C₆ alkoxy-C(O)—, NH₂—C(O)—(CH₂)ₙ—, NH₂—C(O)—, NH₂—C(O)—NH—, OH—C(O)—, NH₂—C(O)—(CH₂)ₙ—NH—C(O)—, (OH)₂—C₁-C₆ alkyl-NH—C(O)—, or OH—C₁-C₆ alkyl-NH—C(O)—;
each R²⁵ is independently H or oxo;
each R²⁶ is independently H, OH, OH—C₁-C₆ alkyl, aryl-(CH₂)ₙ—O—, NH₂—C(O)— or C₁-C₆ alkoxy-C(O)—;
R²⁷ and R²⁸ independently are H, NH₂—C(O)—, or C₃-C₇ cycloalkyl-C(O)—;
R³⁰ is C₁-C₆ alkyl, C₃-C₇ cycloalkyl or NH₂;
R³² is OH;
R³⁵ is independently H, C₁-C₆ alkyl, NH₂—C(O)—, C₁-C₆ alkoxy-C(O)— or C₃-C₇ cycloalkyl;
each R³⁷ is independently H, NH₂C(O)— or OH;
each R³⁸ is independently H, NH₂SO₂—, cyano, heteroaryl, OH, halo, C₁-C₆ alkoxy, OH—C(O)—, or C₁-C₆ alkoxy-C(O)—;
each R⁴¹ independently from H, C₁-C₆ alkoxy or halo;

n is an integer from 1 to 6; and

each R³ is independently H, halo, C₁-C₆ alkyl, aryl, NH₂—C(O)—, C₁-C₆ alkoxy or heteroaryl.
In another embodiment X is CH or N;

R¹ is R⁴₁₋₅-benzyl, R⁵₁₋₅-isoxazolyl-CH₂— or R⁵₁₋₅-pyridinyl-CH₂—; wherein
each R⁴ is H, fluoro, cyano, NH₂—C(O)—;
each R⁵ is independently H or CH₃;

R² is NR¹¹R¹²—C(O)—R¹³CH—, R¹⁴—C(O)—NR¹⁵— CH₂—R¹³CH—, R¹⁶—C(O)—R¹³CH—, (CH₃)₃C—O—C(O)—CH₂—NR¹⁵—C(O)—R¹³CH—, NR¹⁷R¹⁹—C(O)—CH₂—NR¹⁹—C(O)—R¹³CH—, NR¹⁷R¹⁸—C(O)— (CH₂)₂—NR¹⁹—C(O)—R¹³CH—, R²⁹—SO₂—NR²¹—CH₂—R¹³CH—, R²²R²³CH—, R²⁴₁₋₅-dihydroimidazolyl, R²⁴₁₋₅-isoxazolyl, R²⁴₁₋₅-thiadiazolyl, R²⁴₁₋₅-isoxazolyl-R¹³CH—, R²⁴₁₋₅-oxazolyl-R¹³CH—, R²⁴₁₋₅-furyl-R¹³CH—, R²⁴₁₋₅-oxadiazolyl-R¹³CH—, R²⁴₁₋₅-triazolyl-R¹³CH—, R²⁴₁₋₅-dihydroisoxazolyl-R¹³CH—, R²⁴₁₋₅-tetrazolyl-R¹³CH—, R²⁴₁₋₅-isoxazolyl-NR¹⁵—C(O)—R¹³CH—, R²⁴₁₋₅-thiadiazolyl-NR¹⁵—C(O)—R¹³CH—, R²⁵₁₋₅-tetrahydrofuranyl, R²⁵₁₋₅-tetrahydrofuranyl-CH₂—, R²⁶₁₋₅-cyclohexyl, R²⁶₁₋₅-tetrahydronapthyl, R²⁶₁₋₅-dihydroindenyl, NR²⁷R²⁹—(CH₂)₂—NR²⁹—C(O)—R¹³CH—, R³⁰—SO₂—NR³¹—(CH₂)₂—NR¹⁵—C(O)—R¹³CH—, R³⁹—SO₂—(CH₂)₂—NR³¹—C(O)—R¹³CH—, R³²—C(O)—R³³CH—NR³⁴—C(O)—R¹³CH—, R³²—C(O)—(CH₂)₂—NR³⁴—C(O)—R¹³CH—, R³⁵₁₋₅-oxadiazole-(CH₂)₂—NR³⁶—C(O)—R¹³CH—, R³⁵₁₋₅-oxadiazole-CH₂—NR³⁶—C(O)—R¹³CH—, R³⁵₁₋₅-pyridinyl-CH₂—NR³⁶—C(O)—R¹³CH—, R³⁵₁₋₅-tetrazolyl-CH₂—NR³⁶—C(O)—R¹³CH—, R³⁷₁₋₅-tetrahydropyranyl-CH₂—NR³⁶—C(O)—R¹³CH—,

R³⁷₁₋₅-piperidinyl-C(O)—R¹³CH—, R³⁷₁₋₅-pyrrolidinyl-C(O)—R¹³CH—, R³⁷₁₋₅-morpholinyl-(CH₂)₂—NR³⁶—C(O)—R¹³CH—, R³⁷₁₋₅-piperidinyl-(CH₂)₂—NR³⁶—C(O)—R¹³CH—, R³⁷₁₋₅-piperazinyl-(CH₂)₂—NR³⁶—C(O)—R¹³CH—, R³⁷₁₋₅-tertrahydropyranyl-(CH₂)₂—NR³⁶—C(O)—R¹³CH—, R³⁸₁₋₅-phenyl-R³⁹C—NR⁴⁹—C(O)—R¹³CH—, R³⁹₁₋₅-phenyl-(CH₂)₂—NR⁴⁹—C(O)—R¹³CH—, R³⁹₁₋₅-phenyl-(CH₂)₃—NR⁴⁹—C(O)—R¹³CH— or
R⁴¹₁₋₅-benzyl; wherein
R¹¹ and R¹² independently are H, CH₃, (CH₃)₂CH—, cyclobutyl, cyclopropyl, CH₃—O—(CH₂)₂—, OH-ethyl, OH-propyl, (OH)₂-propyl, cyano-CH₂—, (OH—CH₂)₂—CH—, OH-cyclopropyl-CH₂—, OH-cyclopentyl-CH₂—, OH-methyl-cyclopropyl or OH-phenyl;
R¹³ is H, (CH₃)₃C—, (CH₃)₂CHCH₂—, (CH₃)₂CH—, OH-ethyl, benzyl, phenyl, or cyclohexyl;
R¹⁴ is (CH₃CH₂)₂N—, phenyl, (CH₃)₃C—, or cyclopropyl;
R¹⁵, R²¹, R²⁹, R³¹, R³³, R³⁴, R³⁶, R³⁹ and R⁴⁰ are independently H or CH₃;
R¹⁶ is OH or CH₃O;
R¹⁷, R¹⁸ and R¹⁹ are independently H or CH₃;
R²⁰ is (CH₃)₂CH—, CH₃, CF₃, or (CH₃)₂N—;
R²² and R²³ are independently (CH₃)₃C—, (CH₃)₂CH—, cyclohexyl-CH₂—, OHCH₂, phenyl, OH-isopropyl, OH-ethyl, or phenyl-OHCH—;
each R²⁴ is independently H, CH₃, CH₃CH₂—, (CH₃)₃C—, cyclopropyl, CF₃, oxo, NH₂, CH₃CH₂—O—C(O)—, NH₂—C(O)—CH₂—, NH₂—C(O)—, NH₂—C(O)—NH—, OH—C(O)—, NH₂—C(O)—CH₂—NH—C(O)—, (OH)₂-propyl-NH—C(O)— or OH-ethyl-NH—C(O)—;
each R²⁵ is independently H or oxo;
each R²⁶ is independently H, OH, OHCH₂, benzyl-O—, NH₂—C(O)— or CH₃CH₂—O—C(O)—;
R²⁷ and R²⁸ are independently H, NH₂—C(O)—, or cyclopropyl-C(O)—;
R³⁰ is CH₃, cyclopropyl or NH₂;
R³² is OH;
each R³⁵ is independently H, CH₃, NH₂—C(O)—, CH₃CH₂—O—C(O)—, or cyclopropyl;
each R³⁷ is independently H, NH₂C(O)— or OH;
each R³⁸ is independently H, NH₂SO₂—, cyano, tetrazolyl, OH, chloro, CH₃—O—, OH—C(O)—, or CH₃—O—C(O)—;
each R⁴¹ is independently H, CH₃O or fluoro; and

each R³ is independently H, CH₃, chloro, bromo, fluoro, phenyl, NH₂—C(O)—, CH₃O, pyridinyl or oxazolyl.

In another embodiment X is CH or N;

R¹ is

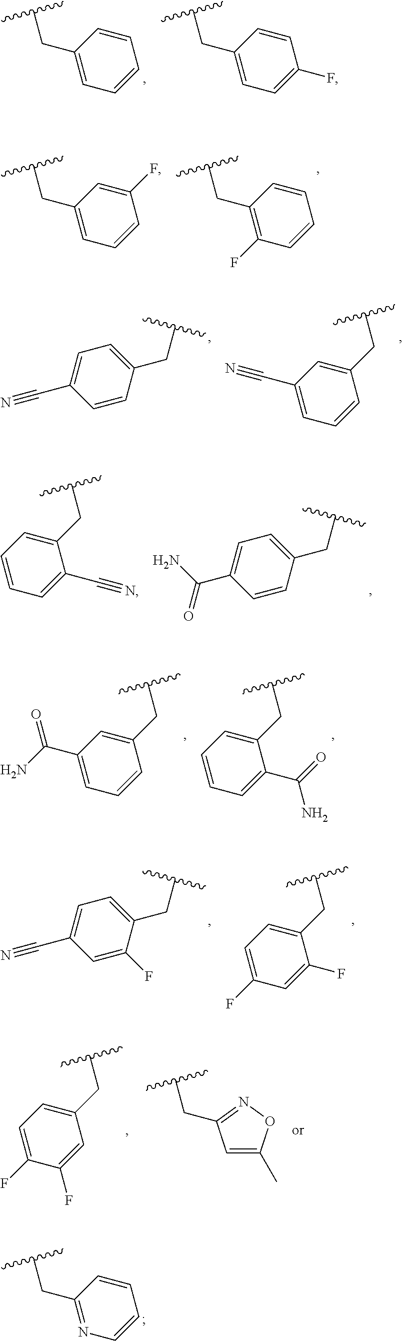

R² is

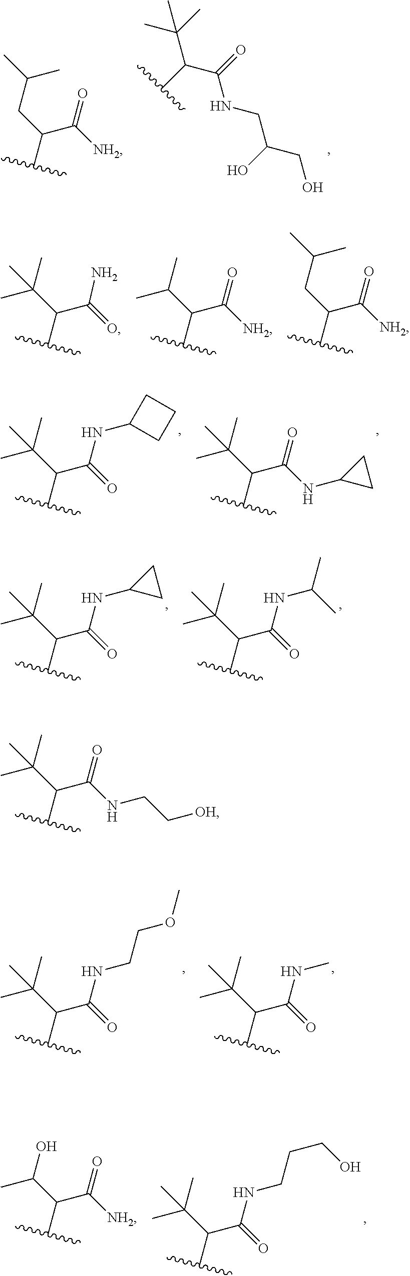

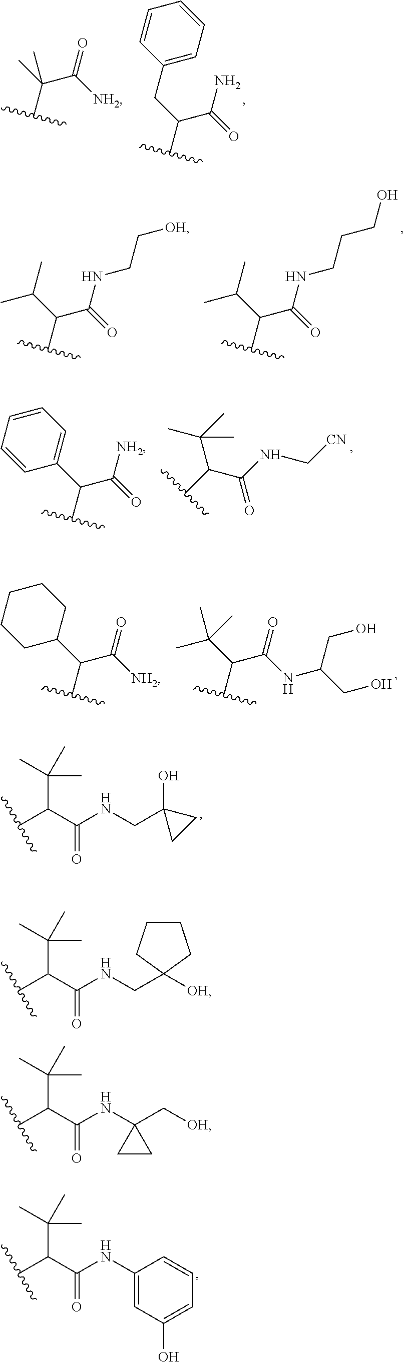

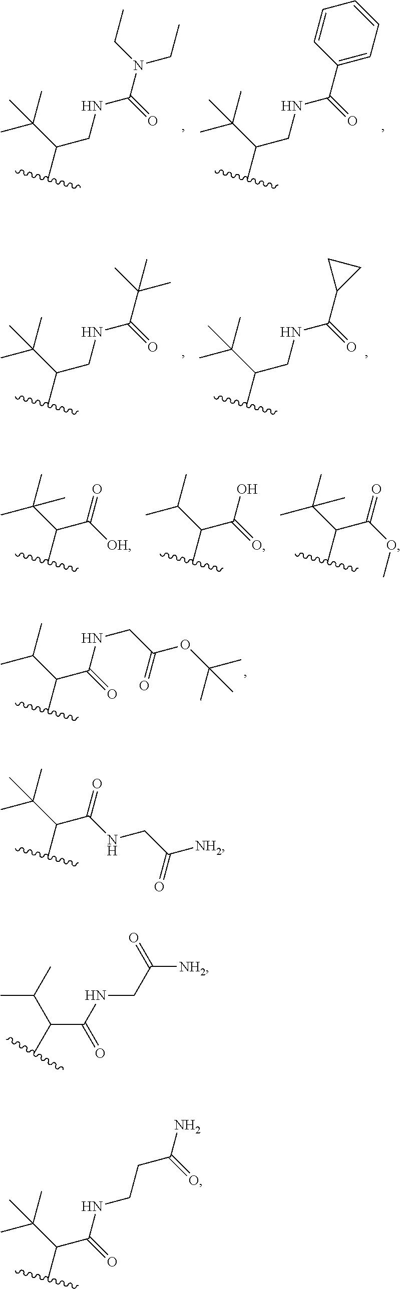

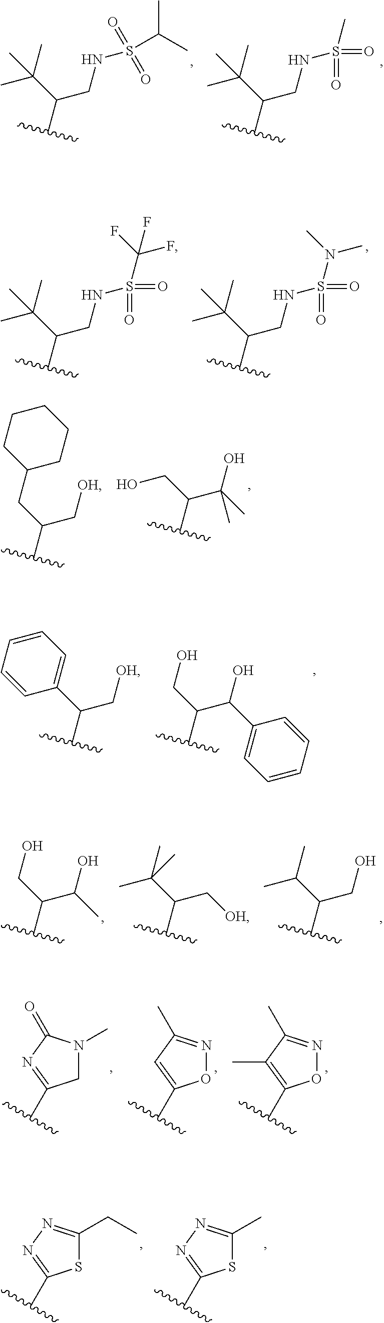

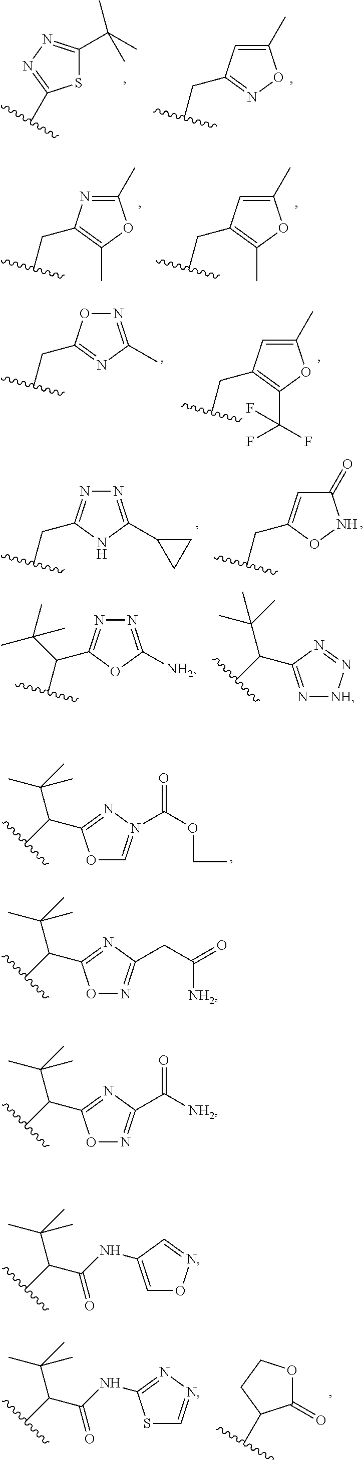

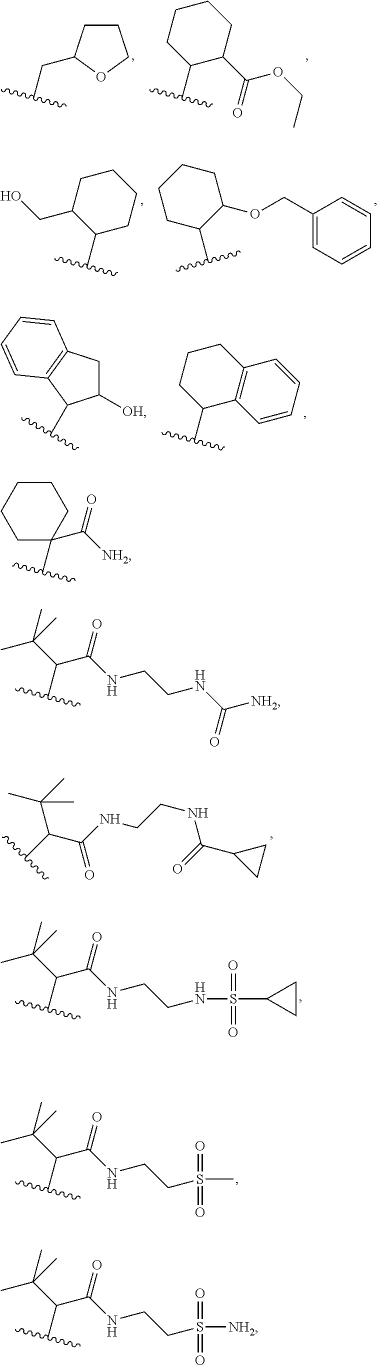

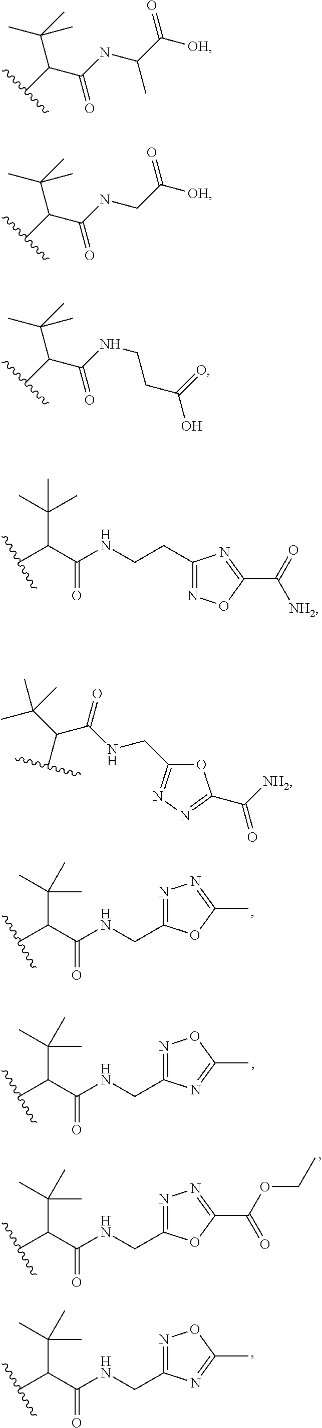

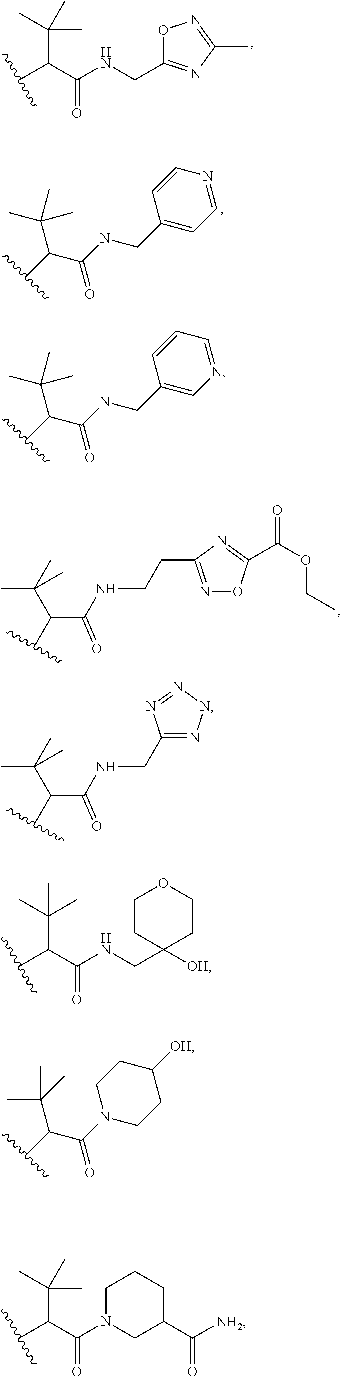

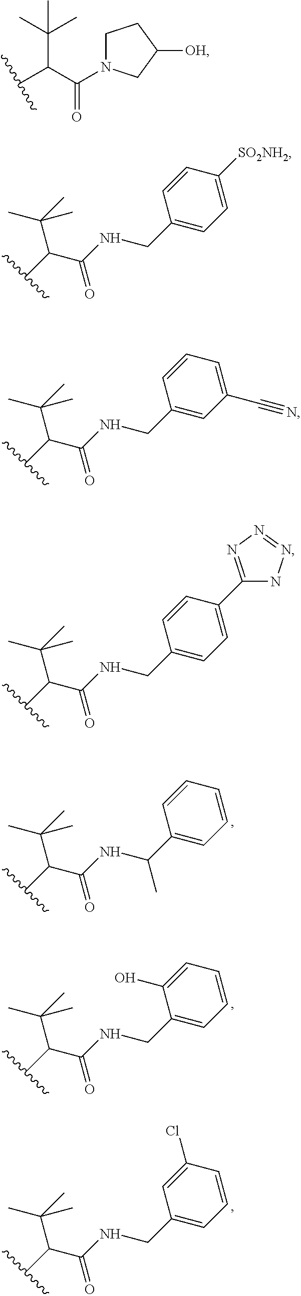

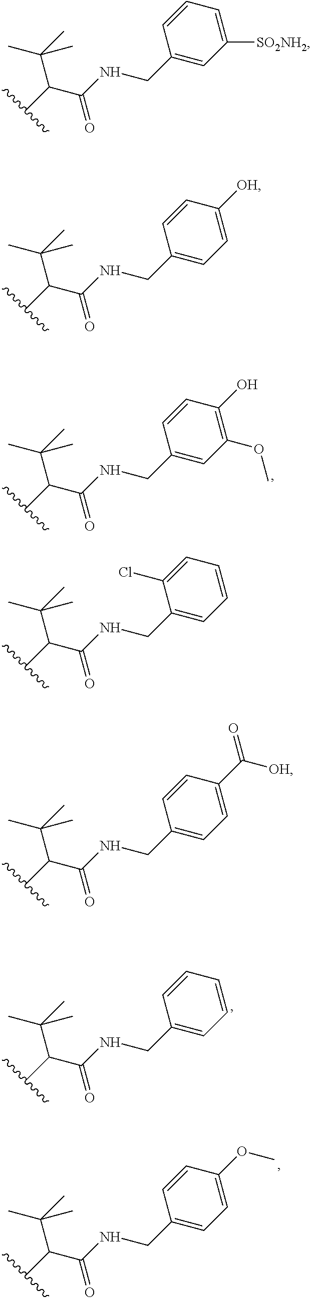

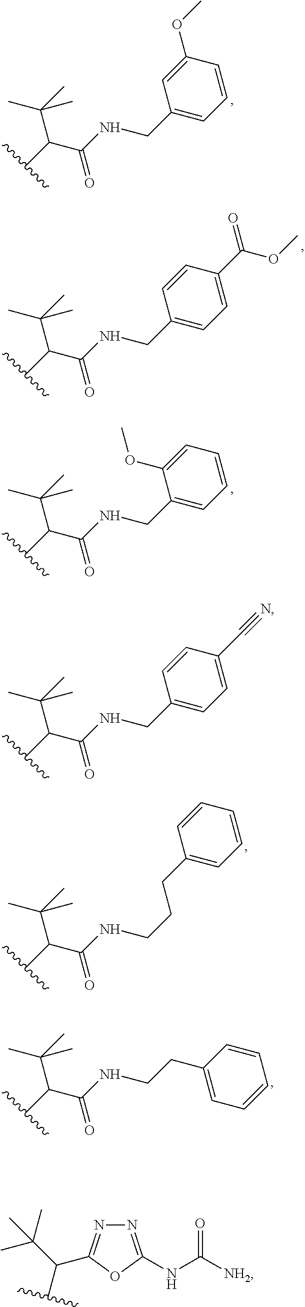

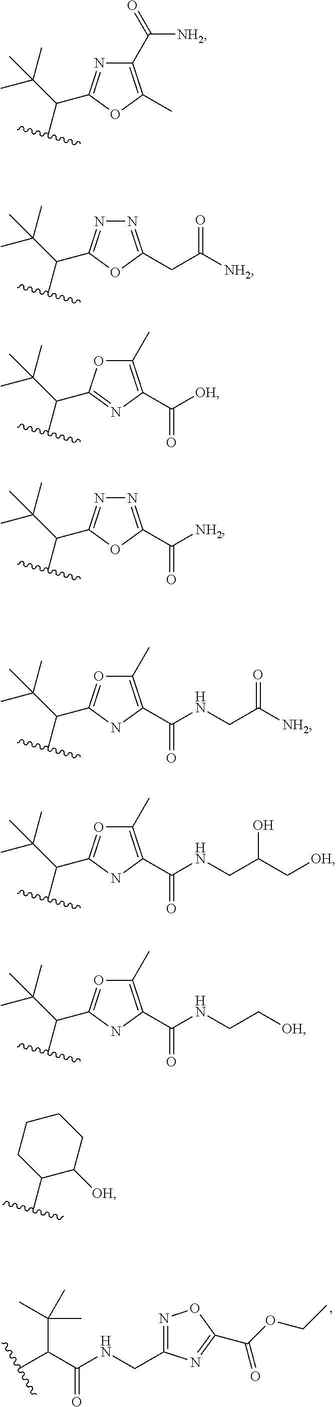

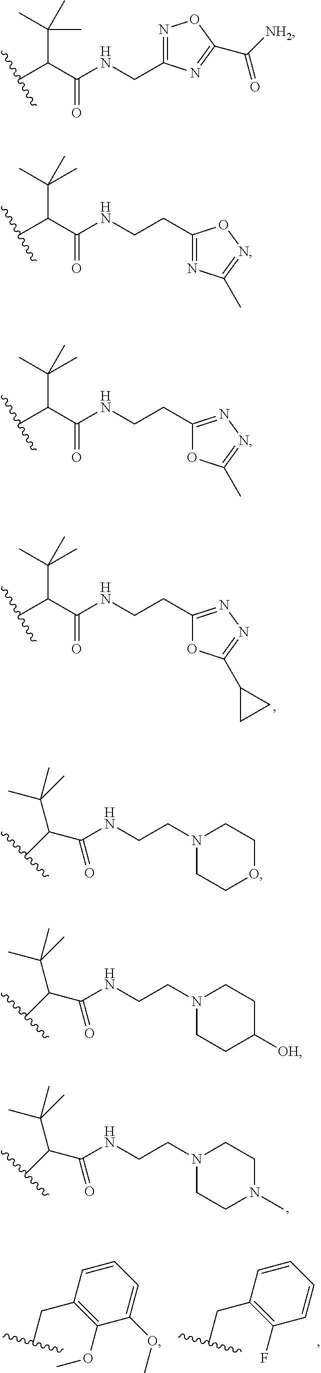

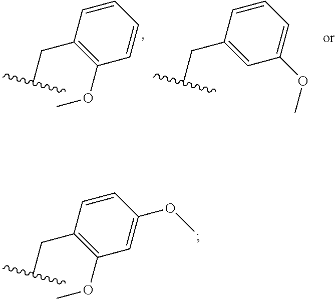

and

each R³ is independently H, CH₃, chloro, bromo, fluoro, phenyl, NH₂—C(O)—, CH₃O—, 3-pyridinyl, 4-pyridinyl, or 2-oxazolyl.

In one embodiment a compound of formula I or a pharmaceutically acceptable salt thereof, wherein

X is CH or N;

R¹ is R⁴₁₋₅-aryl-(CH₂)ₙ— or R⁵₁₋₅-heteroaryl-(CH₂)ₙ—; wherein
each R⁴ is independently H, halo, cyano or NH₂—C(O)—;
each R⁵ is independently H or C₁-C₆ alkyl;

R² is NR¹¹R¹²—C(O)—R¹³CH—, R¹⁶—C(O)—R¹³CH—, NR¹⁷R¹⁸—C(O)—(CH₂)ₙ—NR¹⁹—C(O)—R¹³CH—, R²²R²³CH—, R²⁴₁₋₅-heteroaryl-R¹³CH—, R²⁶₁₋₅—C₃-C₇ cycloalkyl, NR²⁷R²⁸—(CH₂)ₙ—NR²⁹—C(O)—R¹³CH—, R³⁰—SO₂—NR³¹—(CH₂)ₙ—NR¹⁹—C(O)—R¹³CH—, R³⁰—SO₂—(CH₂)ₙ—NR³¹—C(O)—R¹³CH—, R³²—C(O)—R³³CH—NR³⁴—C(O)—R¹³CH—, R³⁵₁₋₅-heteroaryl-(CH₂)ₙ—NR³⁶—C(O)—R¹³CH—,

R³⁷₁₋₅-heterocyclyl-(CH₂)ₙ—NR³⁶—C(O)—R¹³CH—, R³⁷₁₋₅-heterocyclyl-C(O)—R¹³CH— or R⁴¹₁₋₅-aryl-(CH₂)ₙ—; wherein
R¹¹ and R¹² are independently H, C₁-C₆ alkyl, OH—C₁-C₆ alkyl, (OH)₂—C₁-C₆ alkyl, C₁-C₆ alkoxy-(CH₂)ₙ—, C₃-C₇ cycloalkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl, OH-aryl,
R¹³ is H, C₁-C₆ alkyl, OH—C₁-C₆ alkyl, aryl, aryl-(CH₂)ₙ—, or C₃-C₇ cycloalkyl;
R¹⁶ is OH or C₁-C₆ alkoxy;
R¹⁷, R¹⁸ and R¹⁹ are independently H or C₁-C₆ alkyl;
R²² and R²³ are independently C₁-C₆ alkyl, C₃-C₇ cycloalkyl-(CH₂)ₙ—, OH—C₁-C₆ alkyl, or aryl;
each R²⁴ is independently H, C₁-C₆ alkyl, NH₂, NH₂—C(O)—NH—, NH₂—C(O)—, NH₂—C(O)—(CH₂)ₙ—, OH—C(O)—, NH₂—C(O)—(CH₂)ₙ—NH—C(O)—, (OH)₂—C₁-C₆ alkyl-NH—C(O)—, or OH—C₁-C₆ alkyl-NH—C(O)—;
each R²⁶ is independently H, OH, OH—C₁-C₆ alkyl, aryl-(CH₂)ₙ—O—, NH₂—C(O)— or C₁-C₆ alkoxy-C(O)—;
R²⁷ and R²⁸ independently are H or NH₂—C(O)—;
R²⁹R³³, R³⁴, R³⁶ and R³⁸ are independently H or C₁-C₆ alkyl;
R³⁰ is C₁-C₆ alkyl, C₃-C₇ cycloalkyl or NH₂;
R³¹ is H,
R³² is OH;
each R³⁵ is independently H, C₁-C₆ alkyl, NH₂—C(O)—, C₁-C₆ alkoxy-C(O)—, or C₃-C₇ cycloalkyl;
each R³⁷ is independently H, NH₂C(O)— or OH;
each R⁴¹ independently from H, C₁-C₆ alkoxy or halo;

n is an integer from 1 to 6; and

each R³ is independently H, halo, C₁-C₆ alkyl, aryl, NH₂—C(O)—, C₁-C₆ alkoxy or heteroaryl.

In another embodiment X is CH or N;

R¹ is R⁴₁₋₅-benzyl, R⁵₁₋₅-isoxazolyl- CH₂— or R⁵₁₋₅-pyridinyl-CH₂—; wherein
each R⁴ is H, fluoro, cyano, NH₂—C(O)—;
each R⁵ is independently H or CH₃;

R² is NR¹¹R¹²—C(O)—R¹³CH—, R¹⁶—C(O)—R¹³CH—, NR¹⁷R¹⁸—C(O)—CH₂—NR¹⁹—C(O)—R¹³CH—, NR¹⁷R¹⁸—C(O)— (CH₂)₂—NR¹⁹—C(O)—R¹³CH—, R²²R²³CH—, R²⁴₁₋₅-furyl-R¹³CH—, R²⁴₁₋₅-oxadiazolyl-R¹³CH—, R²⁴₁₋₅-tetrazolyl-R¹³CH—, R²⁶₁₋₅-cyclohexyl, R²⁸₁₋₅-tetrahydronapthyl,

R²⁶₁₋₅-dihydroindenyl, NR²⁷R²⁸—(CH₂)₂—NR²⁹—C(O)—R¹³CH—, R³⁹—SO₂—NR³¹—(CH₂)₂—NR¹⁹—C(O)—R¹³CH—, R³⁹—SO₂—(CH₂)₂—NR³¹—C(O)—R¹³CH—, R³²—C(O)—R³³CH—NR³⁴—C(O)—R¹³CH—, R³⁵₁₋₅-oxadiazole-CH₂—NR³⁶—C(O)—R¹³CH—, R³⁵₁₋₅-oxadiazole-(CH₂)₂—NR³⁶—C(O)—R¹³CH—,
R³⁷₁₋₆-morpholinyl-(CH₂)₂—NR³⁶—C(O)—R¹³CH—, R³⁷₁₋₅-piperidinyl-(CH₂)₂—NR³⁶—C(O)—R¹³CH—, R³⁷₁₋₅-piperazinyl-(CH₂)₂—NR³⁶—C(O)—R¹³CH—, R³⁷₁₋₅-tertrahydropyranyl-(CH₂)₂—NR³⁶—C(O)—R¹³CH—, R³⁷₁₋₅-piperidinyl-C(O)—R¹³CH—, R³⁷₁₋₅-pyrrolidinyl-C(O)—R¹³CH— or R⁴¹₁₋₅-benzyl; wherein
R¹¹ and R¹² are independently H, CH₃, (CH₃)₂CH—, cyclobutyl, cyclopropyl,
   CH₃O(CH₂)₂—, OH-ethyl, OH-propyl, (OH)₂-propyl, (OH—CH₂)₂—CH—, OH-cyclopropyl-CH₂—, OH-cyclopentyl-CH₂—, OH—CH₂-cyclopropyl, or OH-phenyl;
   R¹³ is H, (CH₃)₃C, (CH₃)₂CHCH₂—, (CH₃)₂CH—, OH-ethyl, benzyl, phenyl, or cyclohexyl;

### R¹⁶ is OH or CH₃O;

R¹⁷, R¹⁸ and R¹⁹ are independently H or CH₃;
R²² and R²³ are independently (CH₃)₃C—, (CH₃)₂CH—,
   cyclohexyl-CH₂—, OHCH₂, phenyl, OH-isopropyl, or OH-ethyl;
   each R²⁴ is independently H, CH₃, NH₂, NH₂—C(O)—NH—, NH₂—C(O)—, NH₂—C(O)—CH₂—, OH—C(O)—, NH₂—C(O)—CH₂—NH—C(O)—, (OH)₂-propyl-NH—C(O)—, or OH-ethyl-NH—C(O)—; each R²⁶ is independently H, OH, OHCH₂, benzyl-O—, NH₂—C(O)— or CH₃CH₂—O—C(O)—;
R²⁷ and R²⁸ are independently H or NH₂—C(O)—;
R²⁹R³³, R³⁴, R³⁶ and R³⁸ are independently H or CH₃;
R³⁰ is CH₃, cyclopropyl or NH₂;
R³¹ is H,
R³² is OH;
each R³⁵ is independently H, CH₃, NH₂—C(O)—, CH₃CH₂—O—C(O)—, or cyclopropyl;
each R³⁷ is independently H, NH₂C(O)— or OH;
each R⁴¹ is independently H, CH₃O or fluoro; and

each R³ is independently H, CH₃, chloro, bromo, fluoro, phenyl, NH₂—C(O)—, CH₃O, pyridinyl or oxazolyl.

In another embodiment X is CH or N;

R¹ is

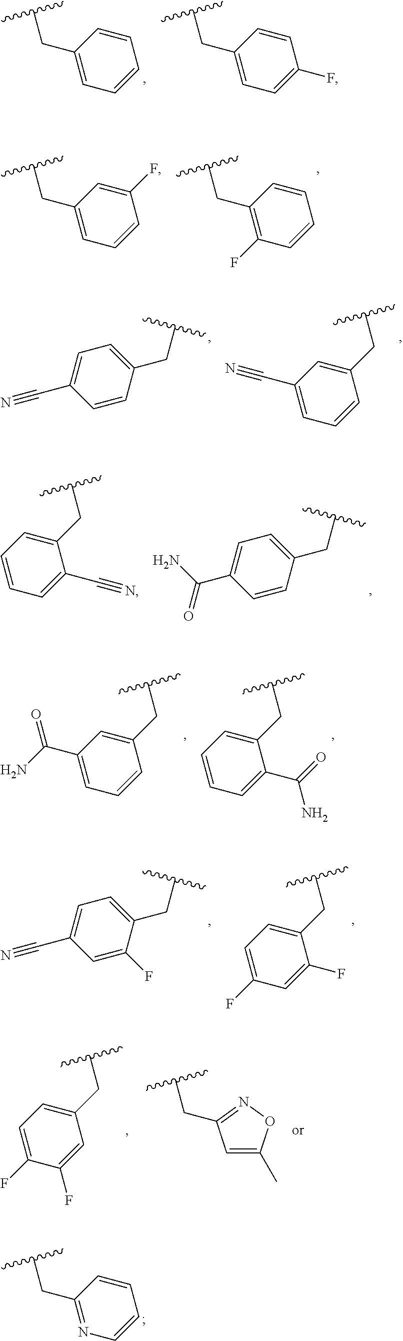

R² is

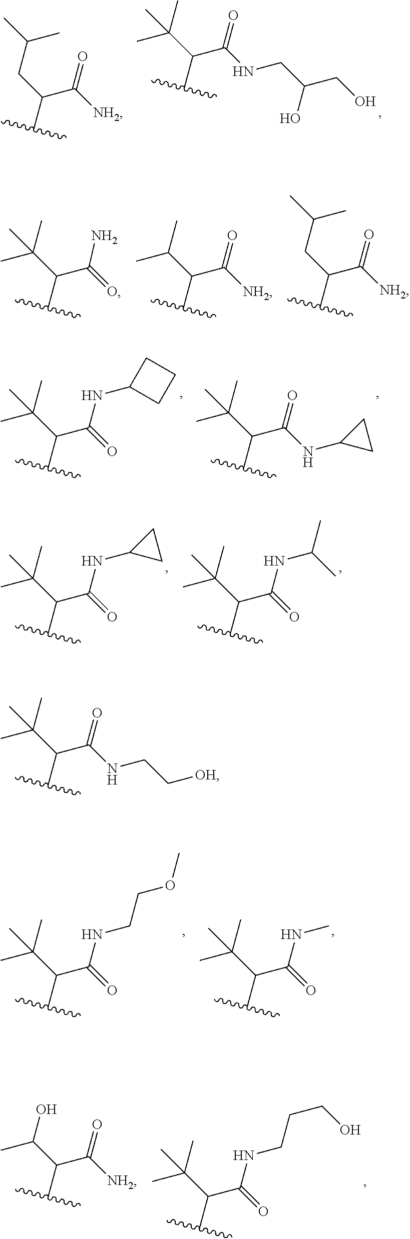

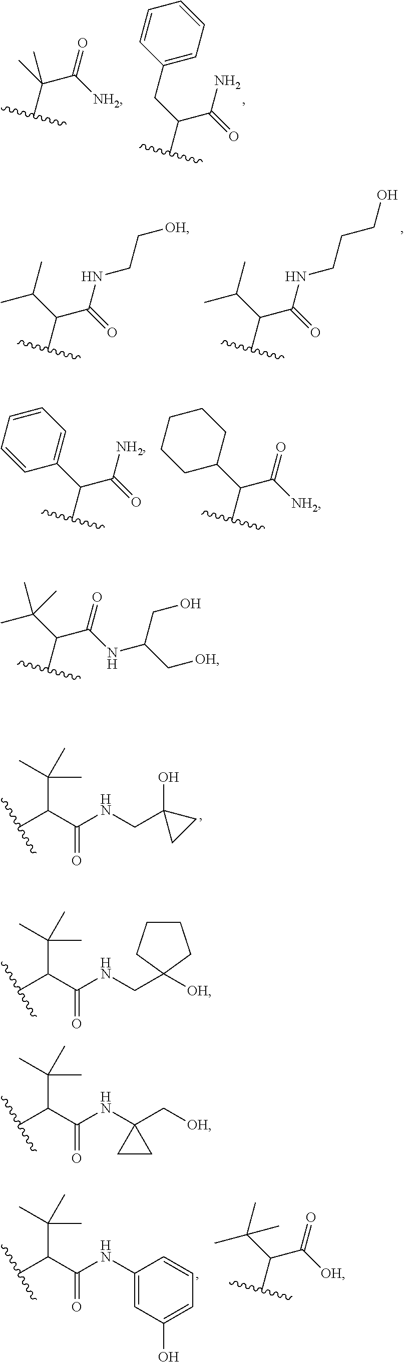

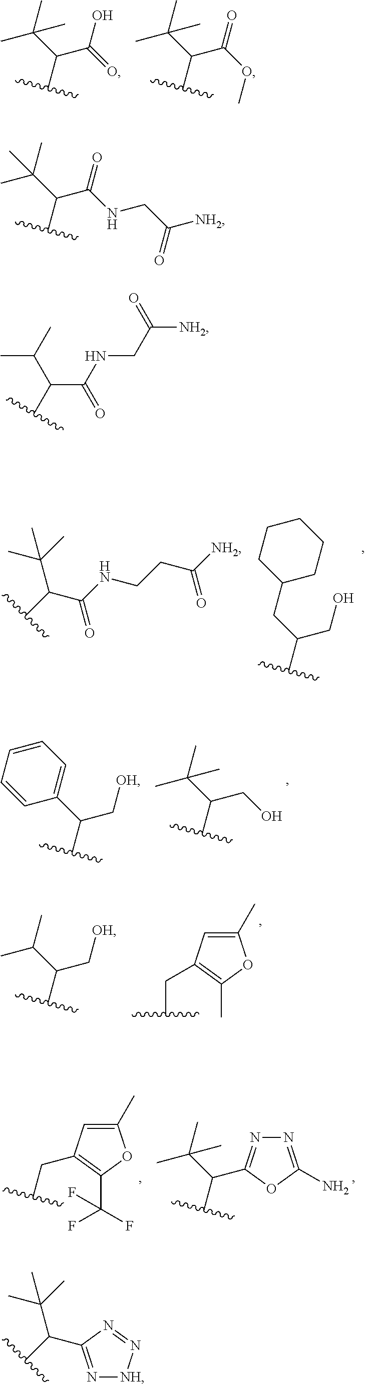

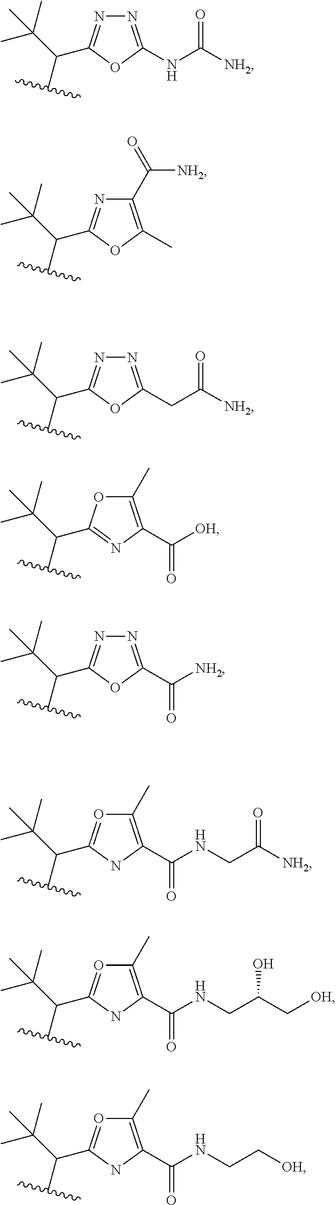

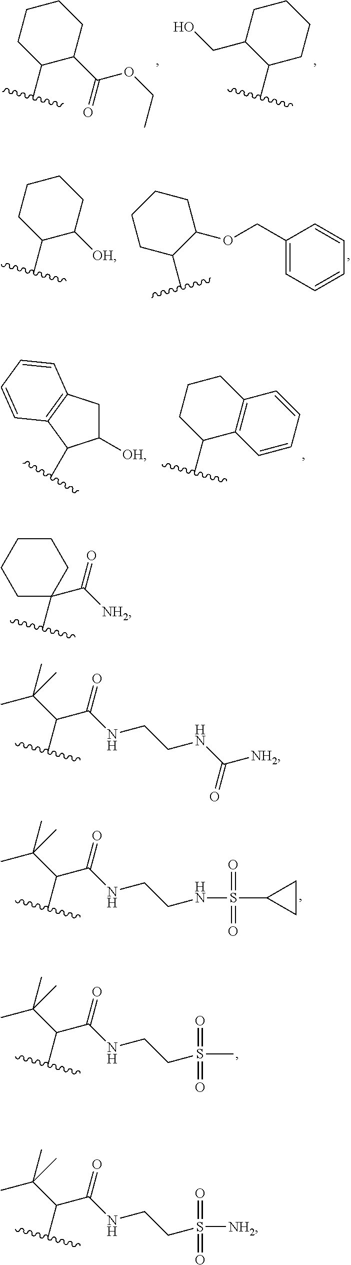

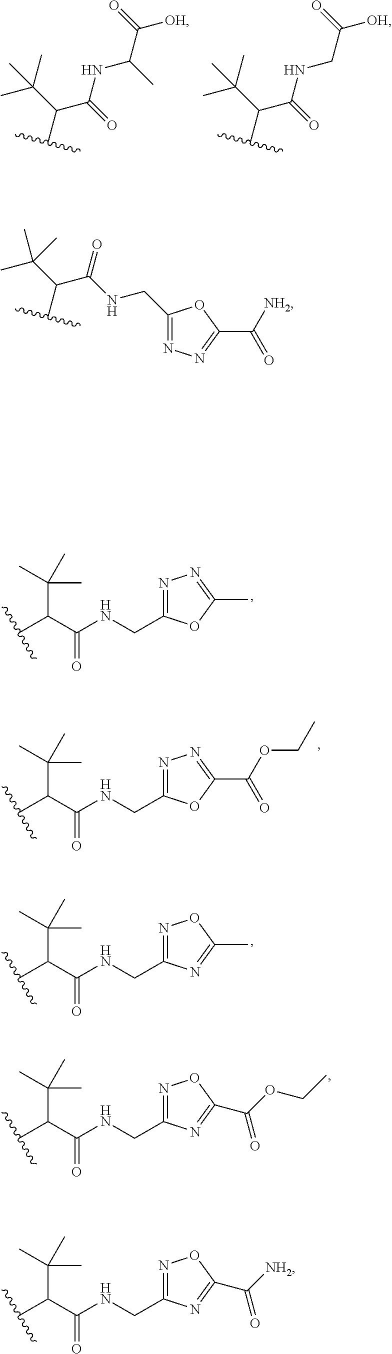

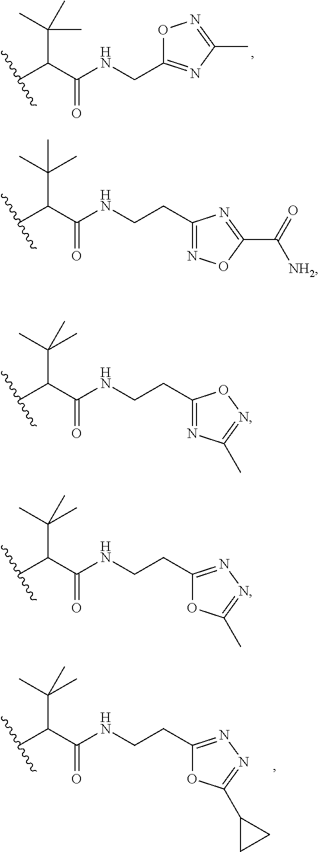

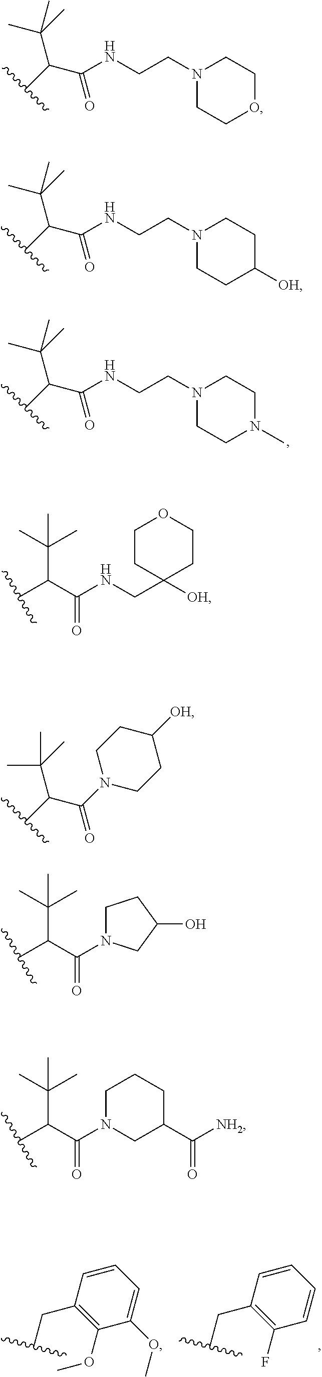

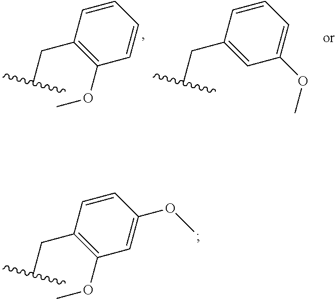

and

each R³ is independently H, CH₃, chloro, bromo, fluoro, phenyl, NH₂—C(O)—, CH₃O, 3-pyridinyl, 4-pyridinyl, or 2-oxazolyl.

In another embodiment X is CH.

In another embodiment X is CH;

R¹ is R⁴₁₋₅-aryl-(CH₂)ₙ— or R⁵₁₋₅-heteroaryl-(CH₂)ₙ—; wherein
each R⁴ is independently H, halo, cyano, or NH₂—C(O)—;
each R⁵ is independently H or C₁-C₆ alkyl;

R² is NR¹¹R¹²—C(O)—R¹³CH—, NR¹⁷R¹⁵—C(O)—(CH₂)ₙ—NR¹⁹—C(O)—R¹³CH—, R²²R²³CH—, R²⁴₁₋₅-heteroaryl-R¹³CH, R³⁰—SO₂—NR³¹—(CH₂)ₙ—NR¹⁹—C(O)—R¹³CH—, R³⁰—SO₂—(CH₂)ₙ—NR³¹—C(O)—R¹³CH— or R³²—C(O)—R³³CH—NR³⁴—C(O)—R¹³CH—; wherein
R¹¹ and R¹² are independently H, OH—C₁-C₆ alkyl, (OH)₂—C₁-C₆ alkyl, C₃-C₇ cycloalkyl or (OH—C₁-C₆ alkyl)₂-(CH₂)ₙ—;
R¹³ is C₁-C₆ alkyl;
R¹⁷, R¹⁸ and R¹⁹ are independently H;
R²² and R²³ are independently C₁-C₆ alkyl or OH—C₁-C₆ alkyl;
each R²⁴ is independently Hor NH₂;
R³⁰ is C₃-C₇ cycloalkyl or NH₂;
R³¹ is H;
R³² is OH;
R³³ is H;
R³⁴ is H;

n is an integer from 1 to 6; and

R³ is H, halo or C₁-C₆ alkyl;

In another embodiment X is CH;

R¹ is

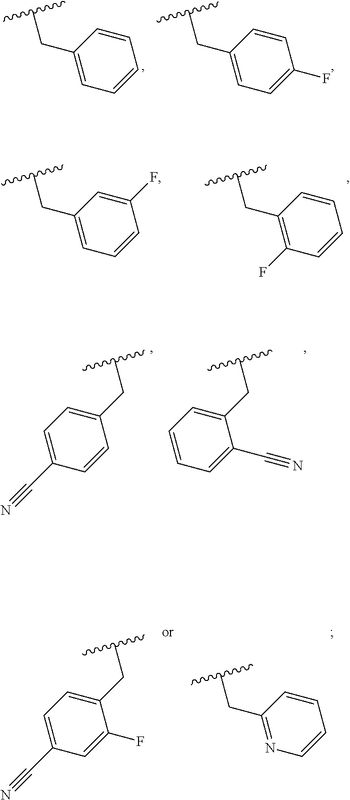

R² is

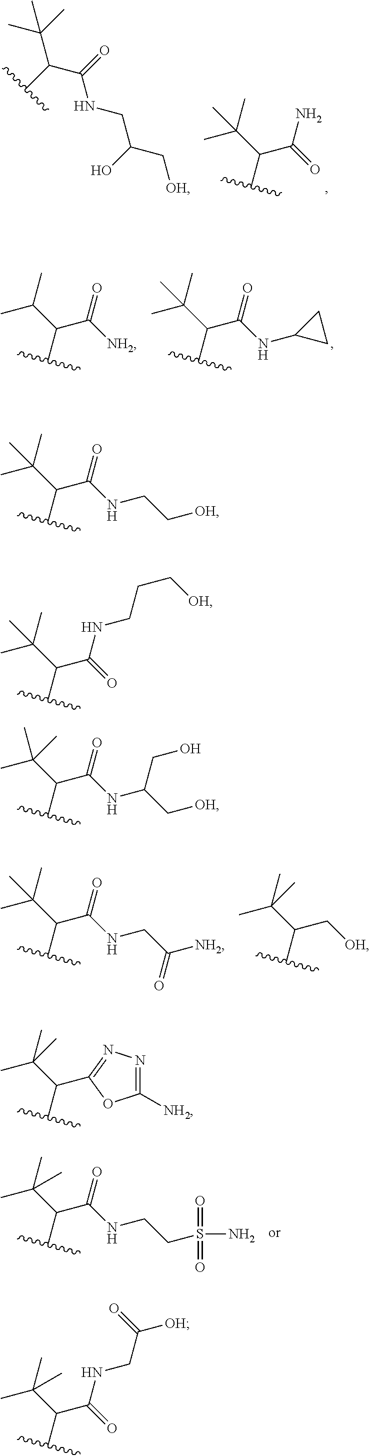

and

R³ is H, F, C₁ or CH₃;

In one embodiment X is N;

R¹ is R⁴₁₋₅-aryl-(CH₂)ₙ— or R⁵₁₋₅-heteroaryl-(CH₂)ₙ—; wherein
each R⁴ is independently H, halo, cyano, or NH₂—C(O)—;
each R⁵ is independently H;

R² is NR¹¹R¹²—C(O)—R¹³CH—, R²²R²³CH— or R¹⁶—C(O)—R¹³CH—; wherein
R¹¹ and R¹² are independently H;
R¹³ is C₁-C₆ alkyl or OH—C₁-C₆ alkyl;
R¹⁶ is OH;
R²² and R²³ are independently C₁-C₆ alkyl or OH—C₁-C₆ alkyl;

n is an integer from Ito 6; and

### R³ is H.

In another embodiment X is N;

R¹ is R⁴₁₋₅-benzyl or R⁵₁₋₅-pyridinyl-CH₂—; wherein
each R⁴ is H or fluoro;
each R⁵ is independently H;

R² is NR¹¹R¹²—C(O)—R¹³CH—, R²²R²³CH— or R¹⁶—C(O)—R¹³CH—; wherein
R¹¹ and R¹² are independently H;
R¹³ is (CH₃)₃C, (CH₃)₂CHCH₂, (CH₃)₂CH₂OH-ethyl;
R¹⁶ is OH;
R²² and R²³ are independently (CH₃)₃C or OHCH₂; and

R³ is H.

In another embodiment X is N;

R¹ is

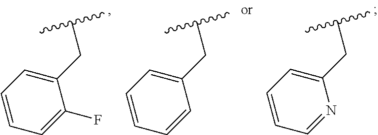

R² is

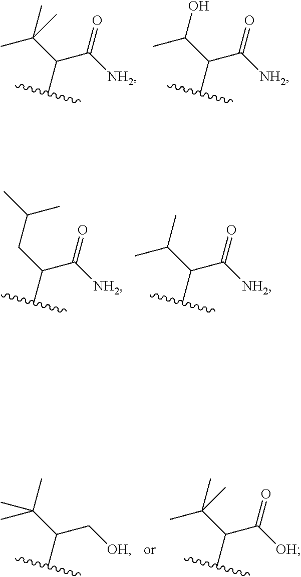

and

R³ is H.

In another embodiment the compound has the general formula

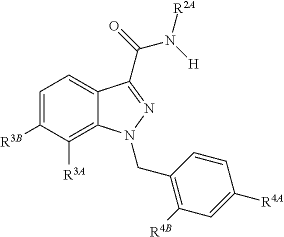

wherein
R^{2A}, is selected from
NR¹¹R¹²—C(O)—R¹³CH—,
C₁-C₆ alkoxy-C(O)—(CH₂)ₙ—NR¹⁵—C(O)—R¹³CH—,
NR¹⁷R¹⁸—C(O)—(CH₂)ₙ—NR¹⁹—C(O)—R¹³CH—,
R²⁴₁₋₅-heteroaryl-NR¹⁵—C(O)—R¹³CH—,
NR²⁷R²⁸—(CH₂)ₙ—NR²⁹—C(O)—R¹³CH—,
R³⁰—SO₂—NR³¹—(CH₂)ₙ—NR¹⁵—C(O)—R¹³CH—,
R³⁰—SO₂—(CH₂)ₙ—NR³¹—C(O)—R¹³CH—,
R³²—C(O)—R³³CH—NR³⁴—C(O)—R¹³CH—,
R³²—C(O)—(CH₂)ₙ—NR³⁴—C(O)—R¹³CH—,
R³⁵₁₋₅-heteroaryl-(CH₂)ₙ—NR³⁶—C(O)—R¹³CH—,
R³⁷₁₋₅-heterocyclyl-(CH₂)ₙ—NR³⁸—C(O)—R¹³CH—,
R³⁷₁₋₅-heterocyclyl-C(O)—R¹³CH—,
R³⁸₁₋₅-aryl-R³⁹C—NR⁴⁰—C(O)—R¹³CH—, or
R³⁸₁₋₅-aryl-(CH₂)ₙ—NR⁴⁰—C(O)—R¹³CH—
wherein
   R¹¹ and R¹² are independently H, C₁-C₆ alkyl, OH—C₁-C₆ alkyl, (OH)₂—C₁-C₆ alkyl, C₁-C₆ alkoxy-(CH₂)ₙ—, C₃-C₇ cycloalkyl, cyano-C₁-C₆ alkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl-, or OH-aryl;
   R¹³ is H, C₁-C₆ alkyl, OH—C₁-C₆ alkyl, aryl, aryl-(CH₂)ₙ—, or C₃-C₇ cycloalkyl;
   R¹⁵, R²⁹, R³¹, R³³, R³⁴, R³⁶, R³⁹ and R⁴⁰ are independently H or C₁-C₆ alkyl;
   R¹⁷, R¹⁸ and R¹⁹ are independently H or C₁-C₆ alkyl;
   each R²⁴ is independently H, C₁-C₆ alkyl, C₃-C₇ cycloalkyl, C₁-C₆ haloalkyl, oxo, NH₂, C₁-C₆ alkoxy-C(O)—, NH₂—C(O)—(CH₂)ₙ—, NH₂—C(O)—, NH₂—C(O)—NH—, OH—C(O)—, NH₂—C(O)—(CH₂)ₙ—NH—C(O)—, (OH)₂—C₁-C₆ alkyl-NH—C(O)—, or OH—C₁-C₆ alkyl-NH—C(O)—;
   each R²⁵ is independently H or oxo;
   R²⁷ and R²⁸ independently are H, NH₂—C(O)—, or C₃-C₇ cycloalkyl-C(O)—;
   R³⁰ is C₁-C₆ alkyl, C₃-C₇ cycloalkyl or NH₂;
   R³² is OH;
   R³⁵ is independently H, C₁-C₆ alkyl, NH₂—C(O)—, C₁-C₆ alkoxy-C(O)— or C₃-C₇ cycloalkyl;
   each R³⁷ is independently H, NH₂C(O)— or OH;
   each R³⁸ is independently H, NH₂SO₂—, cyano, heteroaryl, OH, halo, C₁-C₆ alkoxy, OH—C(O)—, or C₁-C₆ alkoxy-C(O)—;
   n is an integer from 1 to 6;
      R^{3A} and R^{3B} are independently selected from H and halo;
      R^{4A} is selected from F and CN; and
      R^{4B} is selected from H and F.

Preferably, R¹³ is C₁-C₆ alkyl. More preferably it is branched C₃-C₆ alkyl. Most preferably it is tert-butyl.

In another embodiment the compound has the general formula

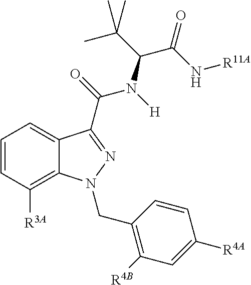

wherein R^{3A} is selected from H, F and Cl, R^{4A} is selected from F and CN, R^{4B} is selected from H and F, and R^{11A} is selected from H, OH—C₁-C₆ alkyl and (OH)₂—C₁-C₆ alkyl.

In another embodiment the compound has the general formula

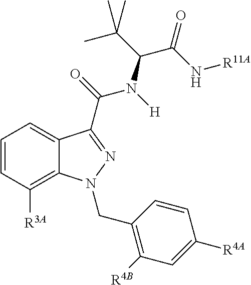

wherein R^{3A} is selected from H, F and Cl, R^{4A} is selected from F and CN, R^{4B} is selected from H and F, and R^{11A} is selected from H, 2-hydroxyethyl and 2,3-dihydroxypropyl.

In one embodiment the compound, or a pharmaceutically acceptable salt thereof, is selected from the group consisting of
N-[(1S)-1-(aminocarbonyl)-2,2-dimethylpropyl]-1-benzyl-5-bromo-1H-indazole-3-carboxamide;
1-[4-(aminocarbonyl)benzyl]-N-[(1S)-1-(aminocarbonyl)-2,2-dimethylpropyl]-1H-indazole-3-carboxamide;
N-[(1S)-1-(aminocarbonyl)-2,2-dimethylpropyl]-1-benzyl-5-pyridin-3-yl-1H-indazole-3-carboxamide;
1-[3-(aminocarbonyl)benzyl]N-[(1S)-1-(aminocarbonyl)-2,2-dimethylpropyl]-1H-indazole-3-carboxamide;
N-[(1S)-1-(aminocarbonyl)-2,2-dimethylpropyl]-1-benzyl-6-bromo-1H-indazole-3-carboxamide;
1-[2-(aminocarbonyl)benzyl]-N-[(1S)-1-(aminocarbonyl)-2,2-dimethylpropyl]-1H-indazole-3-carboxamide;
N-[(1S)-1-(aminocarbonyl)-2,2-dimethylpropyl]-1-benzyl-5-(1,3-oxazol-2-yl)-1H-indazole-3-carboxamide;
N-[(1S)-1-(aminocarbonyl)-2,2-dimethylpropyl]-1-benzyl-5-pyridin-4-yl-1H-indazole-3-carboxamide;
N-[(1S)-1-(aminocarbonyl)-2,2-dimethylpropyl]-1-benzyl-6-pyridin-4-yl-1H-indazole-3-carboxamide;
methyl N-[(1-benzyl-1H-indazol-3-yl)carbonyl]-3-methyl-L-valinate;
1-benzyl-N-(4-methoxybenzyl)-1H-indazole-3-carboxamide;
1-benzyl-N-(2-methoxybenzyl)-1H-indazole-3-carboxamide;
1-benzyl-N-(2-fluorobenzyl)-1H-indazole-3-carboxamide;
1-benzyl-N-(2,3-dimethoxybenzyl)-1H-indazole-3-carboxamide;
1-benzyl-N-(3-methoxybenzyl)-1H-indazole-3-carboxamide;
N-[(1-benzyl-1H-indazol-3-yl)carbonyl]-3-methyl-L-valine;
N-[(1S)-1-(aminocarbonyl)-2,2-dimethylpropyl]-1-benzyl-6-pyridin-3-yl-1H-indazole-3-carboxamide;
N-[(1S)-1-(aminocarbonyl)-2,2-dimethylpropyl]-1-benzyl-5-methoxy-1H-indazole-3-carboxamide;
N˜3˜-[(1S)-1-(aminocarbonyl)-2,2-dimethylpropyl]-1-benzyl-1H-indazole-3,5-dicarboxamide;
N-[(1S)-1-(aminocarbonyl)-2,2-dimethylpropyl]-1-benzyl-6-phenyl-1H-indazole-3-carboxamide;
N-[(1S)-1-(aminocarbonyl)-2,2-dimethylpropyl]-1-benzyl-5-phenyl-1H-indazole-3-carboxamide;
1-(4-cyanobenzyl)-N-{(1S)-1-[(cyclopropylamino)carbonyl]-2,2-dimethylpropyl}-1H-indazole-3-carboxamide;
N-{[1-(4-cyanobenzyl)-1H-indazol-3-yl]carbonyl}-3-methyl-L-valylglycinamide;
1-(4-cyanobenzyl)-N-[(1S)-1-{[(3-hydroxypropyl)amino]carbonyl}-2,2-dimethylpropyl]-1H-indazole-3-carboxamide;
1-(4-cyanobenzyl)-N-[(2,5-dimethyl-3-furyl)methyl]-1H-indazole-3-carboxamide;
1-(4-cyanobenzyl)-N-[(1S)-1-{[(2-hydroxyethyl)amino]carbonyl}-2,2-dimethylpropyl]-1H-indazole-3-carboxamide;
1-(4-cyanobenzyl)-N-[(1S)-2,2-dimethyl-1-(2H-tetrazol-5-yl)propyl]-1H-indazole-3-carboxamide;
N-[(1S)-1-(5-amino-1,3,4-oxadiazol-2-yl)-2,2-dimethylpropyl]-1-(4-cyanobenzyl)-1H-indazole-3-carboxamide;
N-{([1-(4-cyanobenzyl)-1H-indazol-3-yl]carbonyl}-3-methyl-L-valine;
1-benzyl-N-[(1S)-1-({[(2S)-2,3-dihydroxypropyl]amino}carbonyl)-2,2-dimethylpropyl]-1H-indazole-3-carboxamide;
1-benzyl-N-[(1S)-1-({[(2R)-2,3-dihydroxypropyl]amino}carbonyl)-2,2-dimethylpropyl]-1H-indazole-3-carboxamide;
1-benzyl-N-[(1S)-1-{5-[(cyclopropylcarbonyl)amino]-1,3,4-oxadiazol-2-yl}-2,2-dimethylpropyl]-1H-indazole-3-carboxamide;
N-[(1-benzyl-1H-indazol-3-yl)carbonyl]-3-methyl-L-valylglycine;
N-[(1S)-1-({[(2R)-2,3-dihydroxypropyl]amino}carbonyl)-2,2-dimethylpropyl]-1-(4-fluorobenzyl)-1H-indazole-3-carboxamide;
N-[(1S)-1-{5-[(cyclopropylcarbonyl)amino]-1,3,4-oxadiazol-2-yl}-2,2-dimethylpropyl]-1-(4-fluorobenzyl)-1H-indazole-3-carboxamide;
N-[(1S)-1-({[(2S)-2,3-dihydroxypropyl]amino}carbonyl)-2,2-dimethylpropyl]-1-(4-fluorobenzyl)-1H-indazole-3-carboxamide;
N-{(1S)-1-[(cyclopropylamino)carbonyl]-2,2-dimethylpropyl}-1-(4-fluorobenzyl)-1H-indazole-3-carboxamide;
1-(4-fluorobenzyl)-N-[(1S)-1-{[(2-hydroxyethyl)amino]carbonyl}-2,2-dimethylpropyl]-1H-indazole-3-carboxamide;
N-{[1-(4-fluorobenzyl)-1H-indazol-3-yl]carbonyl}-3-methyl-L-valylglycinamide;
N-{[1-(4-fluorobenzyl)-1H-indazol-3-yl]carbonyl}-3-methyl-L-valylglycine;
N-{(1S)-1-[({2-[(aminocarbonyl)amino]ethyl}amino)carbonyl]-2,2-dimethylpropyl}-1-benzyl-1H-indazole-3-carboxamide;
N-{(1S)-1-[({2-[(aminocarbonyl)amino]ethyl}amino)carbonyl]-2,2-dimethylpropyl}-1-(4-cyanobenzyl)-1H-indazole-3-carboxamide;
N-{(1S)-1-[({2-[(aminocarbonyl)amino]ethyl}amino)carbonyl]-2,2-dimethylpropyl}-1-(4-fluorobenzyl)-1H-indazole-3-carboxamide;
N-[(1S)-1-(aminocarbonyl)-2,2-dimethylpropyl]-1-(4-cyano-2-fluorobenzyl)-1H-indazole-3-carboxamide;
1-(4-cyano-2-fluorobenzyl)-N-{(1S)-1-[(cyclopropylamino)carbonyl]-2,2-dimethylpropyl}-1H-indazole-3-carboxamide;
1-(4-cyano-2-fluorobenzyl)-N-[(1S)-1-{5-[(cyclopropylcarbonyl)amino]-1,3,4-oxadiazol-2-yl}-2,2-dimethylpropyl]-1H-indazole-3-carboxamide;
1-(4-cyano-2-fluorobenzyl)-N-[(1S)-1-({[(2R)-2,3-dihydroxypropyl]amino}carbonyl)-2,2-dimethylpropyl]-1H-indazole-3-carboxamide;
1-(4-cyano-2-fluorobenzyl)-N-[(1S)-1-({[(2S)-2,3-dihydroxypropyl]amino}carbonyl)-2,2-dimethylpropyl]-1H-indazole-3-carboxamide;
1-(4-cyano-2-fluorobenzyl)-N-[(1S)-1-{[(2-hydroxyethyl)amino]carbonyl}-2,2-dimethylpropyl]-1H-indazole-3-carboxamide;
N-{[1-(4-cyano-2-fluorobenzyl)-1H-indazol-3-yl]carbonyl}-3-methyl-L-valylglycinamide;
1-(4-cyano-2-fluorobenzyl)-N-[(1S)-1-{[(3-hydroxypropyl)amino]carbonyl}-2,2-dimethylpropyl]-1H-indazole-3-carboxamide;
N-{(1S)-1-[({2-[(aminocarbonyl)amino]ethyl}amino)carbonyl]-2,2-dimethylpropyl}-1-(4-cyano-2-fluorobenzyl)-1H-indazole-3-carboxamide;
N-[(1S)-1-(5-amino-1,3,4-oxadiazol-2-yl)-2,2-dimethylpropyl]-1-(4-cyano-2-fluorobenzyl)-1H-indazole-3-carboxamide;
1-benzyl-N-{(1S)-1-[({2-[(cyclopropylsulfonyl)amino]ethyl}amino)carbonyl]-2,2-dimethylpropyl}-1H-indazole-3-carboxamide;
1-(4-cyanobenzyl)-N-{(1S)-1-[({2-[(cyclopropylsulfonyl)amino]ethyl}amino)carbonyl]-2,2-dimethylpropyl}-1H-indazole-3-carboxamide;
1-(4-cyano-2-fluorobenzyl)-N-{(1S)-1-[({2-[(cyclopropylsulfonyl)amino]ethyl}amino)carbonyl]-2,2-dimethylpropyl}-1H-indazole-3-carboxamide;
N-{(1S)-1-[({2-[(cyclopropylsulfonyl)amino]ethyl}amino)carbonyl]-2,2-dimethylpropyl}-1-(4-fluorobenzyl)-1H-indazole-3-carboxamide;
1-benzyl-N-{(1S)-1-[({2-[(cyclopropylcarbonyl)amino]ethyl}amino)carbonyl]-2,2-dimethylpropyl}-1H-indazole-3-carboxamide;
1-(4-cyanobenzyl)-N-{(1S)-1-[({2-[(cyclopropylcarbonyl)amino]ethyl}amino)carbonyl]-2,2-dimethylpropyl}-1H-indazole-3-carboxamide;
1-(4-cyano-2-fluorobenzyl)-N-{(1S)-1-[({2-[(cyclopropylcarbonyl)amino]ethyl}amino)carbonyl]-2,2-dimethylpropyl}-1H-indazole-3-carboxamide;
N-{(1S)-1-[({2-[(cyclopropylcarbonyl)amino]ethyl}amino)carbonyl]-2,2-dimethylpropyl}-1-(4-fluorobenzyl)-1H-indazole-3-carboxamide;
1-(4-cyanobenzyl)-N-[(1S)-2,2-dimethyl-1-({[2-(methylsulfonyl)ethyl]amino}carbonyl)propyl]-1H-indazole-3-carboxamide;
1-(4-cyano-2-fluorobenzyl)-N-[(1S)-2,2-dimethyl-1-({[2-(methylsulfonyl)ethyl]amino}carbonyl)propyl]-1H-indazole-3-carboxamide;
N-[(1S)-1-({[2-(aminosulfonyl)ethyl]amino}carbonyl)-2,2-dimethylpropyl]-1-(4-cyanobenzyl)-1H-indazole-3-carboxamide;
N-[(1S)-1-({[2-(aminosulfonyl)ethyl]amino}carbonyl)-2,2-dimethylpropyl]-1-(4-cyano-2-fluorobenzyl)-1H-indazole-3-carboxamide;
1-(4-cyanobenzyl)-N-{(1S)-1-[(cyclopropylamino)carbonyl]-2,2-dimethylpropyl}-7-fluoro-1H-indazole-3-carboxamide,
1-(4-cyanobenzyl)-7-fluoro-N-[(1S)-1-{[(2-hydroxyethyl)amino]carbonyl}-2,2-dimethylpropyl]-1H-indazole-3-carboxamide;
1-(4-cyanobenzyl)-7-fluoro-N-[(1S)-1-{[(3-hydroxypropyl)amino]carbonyl}-2,2-dimethylpropyl]-1H-indazole-3-carboxamide;
N-{[1-(4-cyanobenzyl)-7-fluoro-1H-indazol-3-yl]carbonyl}-3-methyl-L-valylglycinamide;
N-[(1S)-1-(aminocarbonyl)-2,2-dimethylpropyl]-1-(4-cyanobenzyl)-7-fluoro-1H-indazole-3-carboxamide;
N-[(1S)-1-(5-amino-1,3,4-oxadiazol-2-yl)-2,2-dimethylpropyl]-1-(4-cyanobenzyl)-7-fluoro-1H-indazole-3-carboxamide;
1-(4-cyanobenzyl)-N-[(1S)-1-({[(2S)-2,3-dihydroxypropyl]amino}carbonyl)-2,2-dimethylpropyl]-7-fluoro-1H-indazole-3-carboxamide;
1-(4-cyanobenzyl)-N-[(1S)-1-({[(2R)-2,3-dihydroxypropyl]amino}carbonyl)-2,2-dimethylpropyl]-7-fluoro-1H-indazole-3-carboxamide;
1-(4-cyanobenzyl)-N-{(1S)-1-[({2-[(cyclopropylsulfonyl)amino]ethyl}amino)carbonyl]-2,2-dimethylpropyl}-7-fluoro-1H-indazole-3-carboxamide
N-{(1S)-1-[({[5-(aminocarbonyl)-1,3,4-oxadiazol-2-yl]methyl}amino)carbonyl]-2,2-dimethylpropyl}-1-(4-cyanobenzyl)-7-fluoro-1H-indazole-3-carboxamide;
1-(4-cyanobenzyl)-7-fluoro-N-[(1S)-1-({[2-hydroxy-1-(hydroxymethyl)ethyl]amino}carbonyl)-2,2-dimethylpropyl]-1H-indazole-3-carboxamide;
N-[(1S)-1-{5-[(aminocarbonyl)amino)]-1,3,4-oxadiazol-2-yl}-2,2-dimethylpropyl]-1-(4-fluorobenzyl)-1H-indazole-3-carboxamide;
N-{(1S)-1-[4-(aminocarbonyl)-5-methyl-1,3-oxazol-2-yl]-2,2-dimethylpropyl}-1-(4-fluorobenzyl)-1H-indazole-3-carboxamide;
N-{(1S)-1-[5-(2-amino-2-oxoethyl)-1,3,4-oxadiazol-2-yl]-2,2-dimethylpropyl}-1-(4-fluorobenzyl)-1H-indazole-3-carboxamide;
2-[(1S)-1-({[1-(4-fluorobenzyl)-1H-indazol-3-yl]carbonyl}amino)-2,2-dimethylpropyl]-5-methyl-1,3-oxazole-4-carboxylic acid;
N-{(1S)-1-[5-(aminocarbonyl)-1,3,4-oxadiazol-2-yl]-2,2-dimethylpropyl}-1-(4-fluorobenzyl)-1H-indazole-3-carboxamide;
N-[(1S)-1-(4-{[(2-amino-2-oxoethyl)amino]carbonyl}-5-methyl-1,3-oxazol-2-yl)-2,2-dimethylpropyl]-1-(4-fluorobenzyl)-1H-indazole-3-carboxamide;
N-{(1S)-1-[4-({[(2S)-2,3-dihydroxypropyl]amino}carbonyl)-5-methyl-1,3-oxazol-2-yl]-2,2-dimethylpropyl}-1-(4-fluorobenzyl)-1H-indazole-3-carboxamide;
1-(4-fluorobenzyl)-N-[(1S)-1-(4-{[(2-hydroxyethyl)amino]carbonyl}-5-methyl-1,3-oxazol-2-yl)-2,2-dimethylpropyl]-1H-indazole-3-carboxamide;
N-[(1S)-2,2-dimethyl-1-({[(5-methyl-1,3,4-oxadiazol-2-yl)methyl]amino}carbonyl)propyl]-1-(4-fluorobenzyl)-1H-indazole-3-carboxamide;
1-(4-cyanobenzyl)-N-[(1S)-2,2-dimethyl-1-({[(5-methyl-1,3,4-oxadiazol-2-yl)methyl]amino}carbonyl)propyl]-1H-indazole-3-carboxamide;
ethyl 5-{[(N-{[1-(4-fluorobenzyl)-1H-indazol-3-yl]carbonyl}-3-methyl-L-valyl)amino]methyl}-1,3,4-oxadiazole-2-carboxylate;
ethyl 5-{[(N-{[1-(4-cyanobenzyl)-1H-indazol-3-yl]carbonyl}-3-methyl-L-valyl)amino]methyl}-1,3,4-oxadiazole-2-carboxylate;
N-{(1S)-1-[({[5-(aminocarbonyl)-1,3,4-oxadiazol-2-yl]methyl}amino)carbonyl]-2,2-dimethylpropyl}-1-(4-fluorobenzyl)-1H-indazole-3-carboxamide;
N-{(1S)-1-[({[5-(aminocarbonyl)-1,3,4-oxadiazol-2-yl]methyl}amino)carbonyl]-2,2-dimethylpropyl}-1-(4-cyanobenzyl)-1H-indazole-3-carboxamide;
N-[(1S)-2,2-dimethyl-1-({[(5-methyl-1,2,4-oxadiazol-3-yl)methyl]amino}carbonyl)propyl]-1-(4-fluorobenzyl)-1H-indazole-3-carboxamide;
1-(4-cyanobenzyl)-N-[(1S)-2,2-dimethyl-1-({[(5-methyl-1,2,4-oxadiazol-3-yl)methyl]amino}carbonyl)propyl]-1H-indazole-3-carboxamide;
1-(4-fluorobenzyl)-N-{(1S)-1-[(4-hydroxypiperidin-1-yl)carbonyl]-2,2-dimethylpropyl}-1H-indazole-3-carboxamide;
1-(4-cyanobenzyl)-N-{(1S)-1-[(4-hydroxypiperidin-1-yl)carbonyl]-2,2-dimethylpropyl}-1H-indazole-3-carboxamide;
ethyl 3-{[(N-{[1-(4-fluorobenzyl)-1H-indazol-3-yl]carbonyl}-3-methyl-L-valyl)amino]methyl}-1,2,4-oxadiazole-5-carboxylate;
ethyl 3-{[(N-{[1-(4-cyanobenzyl)-1H-indazol-3-yl]carbonyl}-3-methyl-L-valyl)amino]methyl}-1,2,4-oxadiazole-5-carboxylate;
N-{(1S)-1-[({[5-(aminocarbonyl)-1,2,4-oxadiazol-3-yl]methyl}amino)carbonyl]-2,2-dimethylpropyl}-1-(4-fluorobenzyl)-1H-indazole-3-carboxamide;
N-{(1S)-1-[({[5-(aminocarbonyl)-1,2,4-oxadiazol-3-yl]methyl}amino)carbonyl]-2,2-dimethylpropyl}-1-(4-cyanobenzyl)-1H-indazole-3-carboxamide;
N-[(1S)-2,2-dimethyl-1-({[(3-methyl-1,2,4-oxadiazol-5-yl)methyl]amino}carbonyl)propyl]-1-(4-fluorobenzyl)-1H-indazole-3-carboxamide;
1-(4-cyanobenzyl)-N-[(1S)-2,2-dimethyl-1-({[(3-methyl-1,2,4-oxadiazol-5-yl)methyl]amino}carbonyl)propyl]-1H-indazole-3-carboxamide;
N-[(1S)-2,2-dimethyl-1-{[(2-morpholin-4-ylethyl)amino]carbonyl}propyl]-1-(4-fluorobenzyl)-1H-indazole-3-carboxamide;
1-(4-fluorobenzyl)-N-[(1S)-1-({[2-(4-hydroxypiperidin-1-yl)ethyl]amino}carbonyl)-2,2-dimethylpropyl]-1H-indazole-3-carboxamide;
N-[(1S)-2,2-dimethyl-1-({[2-(4-methylpiperazin-1-yl)ethyl]amino}carbonyl)propyl]-1-(4-fluorobenzyl)-1H-indazole-3-carboxamide;
N-{(1S)-1-[({2-[5-(aminocarbonyl)-1,2,4-oxadiazol-3-yl]ethyl}amino)carbonyl]-2,2-dimethylpropyl}-1-(4-fluorobenzyl)-1H-indazole-3-carboxamide;
N-{(1S)-1-[({2-[5-(aminocarbonyl)-1,2,4-oxadiazol-3-yl]ethyl}amino)carbonyl]-2,2-dimethylpropyl}-1-(4-cyanobenzyl)-1H-indazole-3-carboxamide;
N-[(1S)-2,2-dimethyl-1-({[2-(3-methyl-1,2,4-oxadiazol-5-yl)ethyl]amino}carbonyl)propyl]-1-(4-fluorobenzyl)-1H-indazole-3-carboxamide;
N-[(1S)-2,2-dimethyl-1-({[2-(5-methyl-1,3,4-oxadiazol-2-yl)ethyl]amino}carbonyl)propyl]-1-(4-fluorobenzyl)-1H-indazole-3-carboxamide;
N-[(1S)-1-({[2-(5-cyclopropyl-1,3,4-oxadiazol-2-yl)ethyl]amino}carbonyl)-2,2-dimethylpropyl]-1-(4-fluorobenzyl)-1H-indazole-3-carboxamide;
1-(4-fluorobenzyl)-N-[(1S)-1-({[(4-hydroxytetrahydro-2H-pyran-4-yl)methyl]amino}carbonyl)-2,2-dimethylpropyl]-1H-indazole-3-carboxamide;
1-(4-cyanobenzyl)-N-[(1S)-1-({[(4-hydroxytetrahydro-2H-pyran-4-yl)methyl]amino}carbonyl)-2,2-dimethylpropyl]-1H-indazole-3-carboxamide;
1-(4-fluorobenzyl)-N-[(1S)-1-{[(3R)-3-hydroxypyrrolidin-1-yl]carbonyl}-2,2-dimethylpropyl]-1H-indazole-3-carboxamide;
1-(4-cyanobenzyl)-N-[(1S)-1-{[(3R)-3-hydroxypyrrolidin-1-yl]carbonyl}-2,2-dimethylpropyl]-1H-indazole-3-carboxamide;
1-(cyclohexylmethyl)-N-[(1S)-1-({[(1-hydroxycyclopropyl)methyl]amino}carbonyl)-2,2-dimethylpropyl]-1H-indazole-3-carboxamide;
1-(4-cyanobutyl)-N-[(1S)-1-({[(1-hydroxycyclopropyl)methyl]amino}carbonyl)-2,2-dimethylpropyl]-1H-indazole-3-carboxamide;
1-(cyclohexylmethyl)-N-[(1S)-1-{[(3-hydroxyphenyl)amino]carbonyl}-2,2-dimethylpropyl]-1H-indazole-3-carboxamide;
1-(4-cyanobutyl)-N-[(1S)-1-{[(3-hydroxyphenyl)amino]carbonyl}-2,2-dimethylpropyl]-1H-indazole-3-carboxamide;
1-(cyclohexylmethyl)-N-[(1S)-1-({[(1-hydroxycyclopentyl)methyl]amino}carbonyl)-2,2-dimethylpropyl]-1H-indazole-3-carboxamide;
1-(4-cyanobutyl)-N-[(1S)-1-({[(1-hydroxycyclopentyl)methyl]amino}carbonyl)-2,2-dimethylpropyl]-1H-indazole-3-carboxamide;
1-(cyclohexylmethyl)-N-[(1S)-1-({[1-(hydroxymethyl)cyclopropyl]amino}carbonyl)-2,2-dimethylpropyl]-1H-indazole-3-carboxamide;
1-(4-fluorobenzyl)-N-[(1S)-1-({[(4-hydroxytetrahydro-2H-pyran-4-yl)methyl]amino}carbonyl)-2,2-dimethylpropyl]-1H-indazole-3-carboxamide;
N-[(1S)-1-{[3-(aminocarbonyl)piperidin-1-yl]carbonyl}-2,2-dimethylpropyl]-1-(cyclohexylmethyl)-1H-indazole-3-carboxamide;
N-[(1S)-1-{[3-(aminocarbonyl)piperidin-1-yl]carbonyl}-2,2-dimethylpropyl]-1-(4-cyanobutyl)-1H-indazole-3-carboxamide;
N-[(1S)-1-(aminocarbonyl)-2,2-dimethylpropyl]-1-(4-cyanobenzyl)-5-fluoro-1H-indazole-3-carboxamide; 1-[4-(aminocarbonyl)benzyl]N-[(1S)-1-(aminocarbonyl)-2,2-dimethylpropyl]-5-fluoro-1H-indazole-3-carboxamide;
1-[4-(aminocarbonyl)benzyl]-5-fluoro-N-[(1S)-1-{[(2-hydroxyethyl)amino]carbonyl}-2,2-dimethylpropyl]-1H-indazole-3-carboxamide;
1-(4-cyanobenzyl)-5-fluoro-N-[(1S)-1-{[(2-hydroxyethyl)amino]carbonyl}-2,2-dimethylpropyl]-1H-indazole-3-carboxamide;
1-(4-cyanobenzyl)-N-{(1S)-1-[(cyclopropylamino)carbonyl]-2,2-dimethylpropyl)-5-fluoro-1H-indazole-3-carboxamide;
N-([1-(4-cyanobenzyl)-5-fluoro-1H-indazol-3-yl]carbonyl}-3-methyl-L-valylglycinamide;
N-{[1-(4-cyanobenzyl)-5-fluoro-1H-indazol-3-yl]carbonyl}-3-methyl-L-valylglycine;
N-[(1S)-1-(aminocarbonyl)-2,2-dimethylpropyl]-5-fluoro-1-(4-fluorobenzyl)-1H-indazole-3-carboxamide;
N-{(1S)-1-[(cyclopropylamino)carbonyl]-2,2-dimethylpropyl}-5-fluoro-1-(4-fluorobenzyl)-1H-indazole-3-carboxamide;
5-fluoro-1-(4-fluorobenzyl)-N-[(1S)-1-{[(2-hydroxyethyl)amino]carbonyl}-2,2-dimethylpropyl]-1H-indazole-3-carboxamide;
N-{[5-fluoro-1-(4-fluorobenzyl)-1H-indazol-3-yl]carbonyl}-3-methyl-L-valylglycinamide;
5-fluoro-1-(4-fluorobenzyl)-N-[(1S)-1-{[(3-hydroxypropyl)amino]carbonyl}-2,2-dimethylpropyl]-1H-indazole-3-carboxamide;
N-[(1S)-1-(aminocarbonyl)-2,2-dimethylpropyl]-7-fluoro-1-(4-fluorobenzyl)-1H-indazole-3-carboxamide;
N-{(1S)-1-[(cyclopropylamino)carbonyl]-2,2-dimethylpropyl}-7-fluoro-1-(4-fluorobenzyl)-1H-indazole-3-carboxamide;
7-fluoro-1-(4-fluorobenzyl)-N-[(1S)-1-{[(2-hydroxyethyl)amino]carbonyl}-2,2-dimethylpropyl]-1H-indazole-3-carboxamide;
7-fluoro-1-(4-fluorobenzyl)-N-[(1S)-1-{[(3-hydroxypropyl)amino]carbonyl}-2,2-dimethylpropyl]-1H-indazole-3-carboxamide;
N-{[7-fluoro-1-(4-fluorobenzyl)-1H-indazol-3-yl]carbonyl}-3-methyl-L-valylglycinamide;
N-{(1S)-1-[({[5-(aminocarbonyl)-1,3,4-oxadiazol-2-yl]methyl}amino)carbonyl]-2,2-dimethylpropyl}-7-fluoro-1-(4-fluorobenzyl)-1H-indazole-3-carboxamide;
N-[(1S)-1-(aminocarbonyl)-2,2-dimethylpropyl]-7-chloro-1-(4-fluorobenzyl)-1H-indazole-3-carboxamide;
7-chloro-N-{(1S)-1-[(cyclopropylamino)carbonyl]-2,2-dimethylpropyl}-1-(4-fluorobenzyl)-1H-indazole-3-carboxamide;
7-chloro-1-(4-fluorobenzyl)-N-[(1S)-1-{[(2-hydroxyethyl)amino]carbonyl}-2,2-dimethylpropyl]-1H-indazole-3-carboxamide;
7-chloro-1-(4-fluorobenzyl)-N-[(1S)-1-{[(3-hydroxypropyl)amino]carbonyl}-2,2-dimethylpropyl]-1H-indazole-3-carboxamide;
N-{[7-chloro-1-(4-fluorobenzyl)-1H-indazol-3-yl]carbonyl}-3-methyl-L-valylglycinamide;
N-{(1S)-1-[({2-[(cyclopropylsulfonyl)amino]ethyl}amino)carbonyl]-2,2-dimethylpropyl}-7-fluoro-1-(4-fluorobenzyl)-1H-indazole-3-carboxamide;
7-chloro-N-{(1S)-1-[({2-[(cyclopropylsulfonyl)amino]ethyl}amino)carbonyl]-2,2-dimethylpropyl}-1-(4-fluorobenzyl)-1H-indazole-3-carboxamide;
N-{[7-fluoro-1-(4-fluorobenzyl)-1H-indazol-3-yl]carbonyl}-3-methyl-L-valylglycine;
N-{[7-fluoro-1-(4-fluorobenzyl)-1H-indazol-3-yl]carbonyl}-3-methyl-L-valyl-D-alanine;
N-{[7-chloro-1-(4-fluorobenzyl)-1H-indazol-3-yl]carbonyl}-3-methyl-L-valyl-D-alanine;
7-chloro-N-[(1S)-1-({[(2S)-2,3-dihydroxypropyl]amino}carbonyl)-2,2-dimethylpropyl]-1-(4-fluorobenzyl)-1H-indazole-3-carboxamide;
N-[(1S)-1-({[(2S)-2,3-dihydroxypropyl]amino}carbonyl)-2,2-dimethylpropyl]-7-fluoro-1-(4-fluorobenzyl)-1H-indazole-3-carboxamide;
7-chloro-N-[(1S)-1-({[(2R)-2,3-dihydroxypropyl]amino}carbonyl)-2,2-dimethylpropyl]-1-(4-fluorobenzyl)-1H-indazole-3-carboxamide;
N-[(1S)-1-({[(2R)-2,3-dihydroxypropyl]amino}carbonyl)-2,2-dimethylpropyl]-7-fluoro-1-(4-fluorobenzyl)-1H-indazole-3-carboxamide;
N-{(1S)-1-[({[5-(aminocarbonyl)-1,3,4-oxadiazol-2-yl]methyl}amino)carbonyl]-2,2-dimethylpropyl}-7-chloro-1-(4-fluorobenzyl)-1H-indazole-3-carboxamide;
N-{[7-chloro-1-(4-fluorobenzyl)-1H-indazol-3-yl]carbonyl}-3-methyl-L-valylglycine;
N-[(1S)-1-(aminocarbonyl)-2,2-dimethylpropyl]-7-chloro-1-(4-cyanobenzyl)-1H-indazole-3-carboxamide;
7-chloro-1-(4-cyanobenzyl)-N-{(1S)-1-[(cyclopropylamino)carbonyl]-2,2-dimethylpropyl}-1H-indazole-3-carboxamide;
7-chloro-1-(4-cyanobenzyl)-N-[(1S)-1-{[(2-hydroxyethyl)amino]carbonyl}-2,2-dimethylpropyl]-1H-indazole-3-carboxamide;
7-chloro-1-(4-cyanobenzyl)-N-[(1S)-1-{[(3-hydroxypropyl)amino]carbonyl}-2,2-dimethylpropyl]-1H-indazole-3-carboxamide;
N-{[7-chloro-1-(4-cyanobenzyl)-1H-indazol-3-yl]carbonyl}-3-methyl-L-valylglycinamide;
7-chloro-1-(4-cyanobenzyl)-N-{(1S)-1-[({2-[(cyclopropylsulfonyl)amino]ethyl}amino)carbonyl]-2,2-dimethylpropyl}-1H-indazole-3-carboxamide;
7-chloro-1-(4-cyanobenzyl)-N-[(1S)-1-({[(2S)-2,3-dihydroxypropyl]amino}carbonyl)-2,2-dimethylpropyl]-1H-indazole-3-carboxamide;
7-chloro-1-(4-cyanobenzyl)-N-[(1S)-1-({[(2R)-2,3-dihydroxypropyl]amino}carbonyl)-2,2-dimethylpropyl]-1H-indazole-3-carboxamide;
N-{[7-chloro-1-(4-cyanobenzyl)-1H-indazol-3-yl]carbonyl}-3-methyl-L-valylglycine;
N-{[7-chloro-1-(4-cyanobenzyl)-1H-indazol-3-yl]carbonyl}-3-methyl-L-valyl-D-alanine;
N-{[1-(3-fluorobenzyl)-1H-indazol-3-yl]carbonyl}-3-methyl-L-valylglycine;
N-{[1-(2-fluorobenzyl)-1H-indazol-3-yl]carbonyl}-3-methyl-L-valylglycine;
N-{[1-(2,4-difluorobenzyl)-1H-indazol-3-yl]carbonyl}-3-methyl-L-valylglycine; or
N-{[1-(3,4-difluorobenzyl)-1H-indazol-3-yl]carbonyl}-3-methyl-L-valylglycine.

In one embodiment the compound, or a pharmaceutically acceptable salt thereof, is selected from the group consisting of
N-[(1S)-1-(aminocarbonyl)-2,2-dimethylpropyl]-1-(2-fluorobenzyl)-1H-pyrazolo[3,4-b]pyridine-3-carboxamide;
N-[(1S,2R)-1-(aminocarbonyl)-2-hydroxypropyl]-1-(2-fluorobenzyl)-1H-pyrazolo[3,4-b]pyridine-3-carboxamide;
N-[(1S)-1-(aminocarbonyl)-3-methylbutyl]-1-(2-fluorobenzyl)-1H-pyrazolo[3,4-b]pyridine-3-carboxamide;
1-(2-fluorobenzyl)-N-[(1S)-1-(hydroxymethyl)-2,2-dimethylpropyl]-1H-pyrazolo[3,4-b]pyridine-3-carboxamide;
N-[(1S)-1-(aminocarbonyl)-2,2-dimethylpropyl]-1-(pyridin-2-ylmethyl)-1H-pyrazolo[3,4-b]pyridine-3-carboxamide;
N-[(1S)-1-(aminocarbonyl)-2-methylpropyl]-1-(pyridin-2-ylmethyl)-1H-pyrazolo[3,4-b]pyridine-3-carboxamide;
N-[(1S)-1-(aminocarbonyl)-3-methylbutyl]-1-(pyridin-2-ylmethyl)-1H-pyrazolo[3,4-b]pyridine-3-carboxamide;
N-[(1-benzyl-1H-pyrazolo[3,4-b]pyridin-3-yl)carbonyl]-3-methyl-L-valine;
N-[(1S)-1-(aminocarbonyl)-2,2-dimethylpropyl]-1-benzyl-1H-pyrazolo[3,4-b]pyridine-3-carboxamide;
N-[(1S)-1-(aminocarbonyl)-2-methylpropyl]-1-benzyl-1H-pyrazolo[3,4-b]pyridine-3-carboxamide;
1-benzyl-N-[(1S)-1-(hydroxymethyl)-2,2-dimethylpropyl]-1H-pyrazolo[3,4-b]pyridine-3-carboxamide;
N-[(1S)-1-(aminocarbonyl)-3-methylbutyl]-1-benzyl-1H-pyrazolo[3,4-b]pyridine-3-carboxamide;
N-[(1S,2R)-1-(aminocarbonyl)-2-hydroxypropyl]-1-benzyl-1H-pyrazolo[3,4-b]pyridine-3-carboxamide;
N-[(1S)-1-(hydroxymethyl)-2,2-dimethylpropyl]-1-(pyridin-2-ylmethyl)-1H-pyrazolo[3,4-b]pyridine-3-carboxamide;
N-[(1S,2R)-1-(aminocarbonyl)-2-hydroxypropyl]-1-(pyridin-2-ylmethyl)-1H-pyrazolo[3,4-b]pyridine-3-carboxamide; or
N-[(1S)-1-(aminocarbonyl)-2-methylpropyl]-1-(2-fluorobenzyl)-1H-pyrazolo[3,4-b]pyridine-3-carboxamide.

In one embodiment the present invention is a pharmaceutical composition comprising a compound of Formula I or a pharmaceutically acceptable salt, enantiomer, or racemate thereof.

In one embodiment the present invention is a method for the treatment of a CB1 mediated disorder in a subject in need of such treatment or prevention, wherein the method comprises administering to the subject an amount of a compound of Formula I or a pharmaceutically acceptable salt, enantiomer, or racemate thereof, wherein the amount of the compound is effective for the treatment or prevention of the CB1 mediated disorder.

In one embodiment the CB1 mediated disorder is pain.

### Salts of the Compounds of this Invention

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the compounds of this invention may be prepared from an inorganic or organic acid. Examples of suitable inorganic acids include hydrochloric, hydrobromic acid, hydroionic, nitric, carbonic, sulfuric, and phosphoric acid. Suitable organic acids generally include, for example, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, undecanoate and naphthalene-1,5-disulfonate.

Pharmaceutically-acceptable base addition salts of the compounds of this invention include, for example, metallic salts and organic salts. Preferred metallic salts include alkali metal (group Ia) salts, alkaline earth metal (group IIa) salts, and other physiological acceptable metal salts. Such salts may be made from aluminum, calcium, lithium, magnesium, potassium, sodium, and zinc. Preferred organic salts may be made from tertiary amines and quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

Also within the scope of the invention are so-called ‘prodrugs’ of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985). Some examples of prodrugs in accordance with the invention include:
(i) where the compound of formula (I) contains an alcohol functionality (—OH), an ether thereof, for example, replacement of the hydrogen with (C₁-C₆)alkanoyloxymethyl;
(ii) where the compound of formula (I) contains carboxy group, an ester thereof, for example, replacement of the OH of the carboxy with C₁-C₈ alkyl; and
(ii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

Compounds of formula (I) containing one or more asymmetric carbon atoms can exist as two or more stereoisomers. Where the compound contains, for example, a keto or oxime group or an aromatic moiety, tautomeric isomerism ('tautomerism') can occur. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

All of the compounds of the formula (I) can be prepared by the procedures described in the general methods presented below or by the specific methods described in the Examples section and the Preparations section, or by routine modifications thereof. The present invention also encompasses any one or more of these processes for preparing the compounds of formula (I), in addition to any novel intermediates used therein.

### Treating Conditions Using the Compounds of this Invention

The method of the present invention is useful for, but not limited to, the treatment of disorders that are mediated by CB1 in a subject. For example, the compounds described herein would be useful for the treatment of any symptoms associated with a CB1 meditated disorder described below.

As used herein, the terms “treating”, “treatment”, “treated”, or “to treat,” can be used interchangeably. Treatment includes palliative treatment, preventive treatment and restorative treatment. Palliative treatment includes alleviation, elimination of causation of pain and/or inflammation associated with a CB1 mediated disorder. Preventative treatment means to prevent or to slow the appearance of symptoms associated with a CB1 mediated disorder. For methods of prevention, the subject is any subject, and preferably is a subject that is in need of prevention of a CB1 mediated disorder.

The term “subject” for purposes of treatment includes any human or animal subject who is in need of the prevention of, or who has a TNFα-mediated inflammatory disease or disorder. The subject is typically a mammal.

In some embodiments, the methods and compositions of the present invention encompass the treatment of conditions including pain and neurodegenerative disorders. (See Annu. Rev. Pharmacol. Toxicol. (2006) 46:101-22; Clinical Neuroscience Research (2005) δ 185-199; Prostaglandins, Leukotrienes and Essential Fatty Acids (2002) 66(2&3), 101-121.)

In some embodiments, the methods and compositions of the present invention encompass the treatment of pain, including but not limited to chronic pain, acute pain, joint pain, nociceptive pain, neuropathic pain, allodynia, hyperalgesia, burn pain, menstrual cramps, kidney stones, headache, migraine headache, sinus headaches, tension headaches, dental pain, myasthenia gravis, rheumatoid arthritic pain, osteoarthritic pain, back pain, cancer pain, multiple sclerosis, sarcoidosis, Behcet's syndrome, myositis, polymyositis, gingivitis, hypersensitivity, swelling occurring after injury, closed head injury, endometriosis, stroke, and the like.

In other embodiments, the methods and compositions of the present invention encompass the treatment of the connective tissue and joint disorders selected from the group consisting of osteoarthritis, rheumatoid arthritis, ankylosing spondylitis, fibromyalgia, spondyloarthopathies, gouty arthritis, lumbar spondylarthrosis, carpal tunnel syndrome, psoriatic arthritis, sclerodoma, canine hip dysplasia, systemic lupus erythematosus, juvenile arthritis, osteoarthritis, tendonitis and bursitis.

In other embodiments, the methods and compositions of the present invention encompass the treatment of neurological dosirders including neuroinflammation and neurodegenerative disorders selected from the group consisting of neuritis, Alzheimer's disease, multiple sclerosis (MS), Parkinson's disease, Tourette's syndrome, spasticity and epilepsy.

In other embodiments, the methods and compositions of the present invention encompass the treatment of neuropathies including HIV related neuropathy, nerve injury, spinal cord injury, sciatica, neuralgia, diabetic neuropathy, nerve pain, and some peripheral neuropathies and neurodegenerative disorders.

In other embodiments, the methods and compositions of the present invention encompass the treatment of the respiratory disorders selected from the group consisting of cough, asthma, bronchitis, chronic obstructive pulmonary disease (COPD), broncho constriction, cystic fibrosis, pulmonary edema, pulmonary embolism, pneumonia, pulmonary sarcoisosis, silicosis, pulmonary fibrosis, respiratory failure, acute respiratory distress syndrome, seasonal allergic rhinitis, reversible airway obstruction, adult respiratory disease syndrome, cryptogenic fibrosing alveolitis and emphysema.

In other embodiments, the methods and compositions of the present invention encompass the treatment of the dermatological disorders selected from the group consisting of acne, psoriasis, eczema, burns, poison ivy, poison oak and dermatitis.

In other embodiments, the methods and compositions of the present invention encompass the treatment of the surgical disorders selected from the group consisting of pain and swelling following surgery, infection following surgery and inflammation following surgery.

In other embodiments, the methods and compositions of the present invention encompass the treatment of the gastrointestinal disorders selected from the group consisting of colitis, inflammatory bowel disease, irritable bowel syndrome, Crohn's disease, gastritis, irritable bowel syndrome, diarrhea, constipation, dysentery, ulcerative colitis, gastric esophageal reflux, gastric ulcers, gastric varices, ulcers, functional gastrointestinal disorder, and heartburn.

In other embodiments, the methods and compositions of the present invention encompass the treatment of the ophthalmic disorders selected from the group consisting of retinopathies, uveitis, ocular photophobia, acute injury to the eye tissue, conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocorneal endothelial syndrome, keratoconus, lattice dystrophy, map-dot-fingerprint dystrophy, ocular herpes, pterygium, myopia, hyperopia, and cataracts.

Cannabinoid agonists are believed to be useful in the treatment of other disorders including acute cerebral ischemia, neuroprotection, anxiety, cerebrovascular ischemia, cachexia, nausea, emesis, chemotherapy-induced emesis, cutaneous T cell lymphoma, diabetes, osteoporosis, glomerulonephritis, renal ischemia, nephritis, hepatitis, cerebral stroke, vasodialation, hypertension, vasculitis, myocardial infarction and cerebral ischemia.

### Pharmaceutical Compositions Containing the Compounds of this Invention

This invention also is directed to pharmaceutical compositions (or “medicaments”) comprising the compounds described above (including tautomers of the compounds, and pharmaceutically-acceptable salts of the compounds and tautomers), and to methods for making pharmaceutical compositions comprising those compounds in combination with one or more conventional non-toxic, pharmaceutically-acceptable carriers, diluents, wetting or suspending agents, vehicles, and/or adjuvants (the carriers, diluents, wetting or suspending agents, vehicles, and adjuvants sometimes being collectively referred to in this specification as “carrier materials”); and/or other active ingredients. The preferred composition depends on the method of administration. Formulation of drugs is generally discussed in, for example, Hoover, John E., Remington's Pharmaceutical Sciences (Mack Publishing Co., Easton, Pa.: 1975) (incorporated by reference into this specification). See also, Liberman, H. A., Lachman, L., eds., Pharmaceutical Dosage Forms (Marcel Decker, New York, N.Y., 1980) (incorporated by reference into this specification).

In many embodiments, the pharmaceutical composition is made in the form of a dosage unit containing a particular amount of the active ingredient. Typically, the pharmaceutical composition contains from about 0.1 to 1000 mg (and more typically, 7.0 to 350 mg) of the compound.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of the invention. The overall daily dose will typically be in the range 0.001 mg to 40 mg which may be administered in a single dose or, more usually, as divided doses throughout the day.

Solid dosage forms for oral administration include, for example, hard or soft capsules, tablets, pills, powders, and granules. In such solid dosage forms, the compounds are ordinarily combined with one or more adjuvants. If administered per os, the compounds may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation, as may be provided in a dispersion of the compound of this invention in hydroxypropylmethyl cellulose. In the case of capsules, tablets, and pills, the dosage forms also may comprise buffering agents, such as sodium citrate, or magnesium or calcium carbonate or bicarbonate. Tablets and pills additionally may be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable carrier materials include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non-ionic detergents), and/or polyethylene glycols (e.g., PEG 400).

Formulations for parenteral administration may, for example, be prepared from sterile powders or granules having one or more of the carriers materials mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

### General Synthesis

Compounds of formula (I) illustrated in the Examples hereinafter, and the requisite intermediates for preparing the compounds of formula (I), may be prepared using the methods described in the following Schemes A and B. The skilled man will appreciate that the compounds of the invention could be made by methods other than those specifically described herein, for example by adaptation of the herein described methods according to the known art. In the methods below, unless otherwise specified, the groups X, R¹, R², and R³₁₋₄ are as described above for a compound of formula (I).

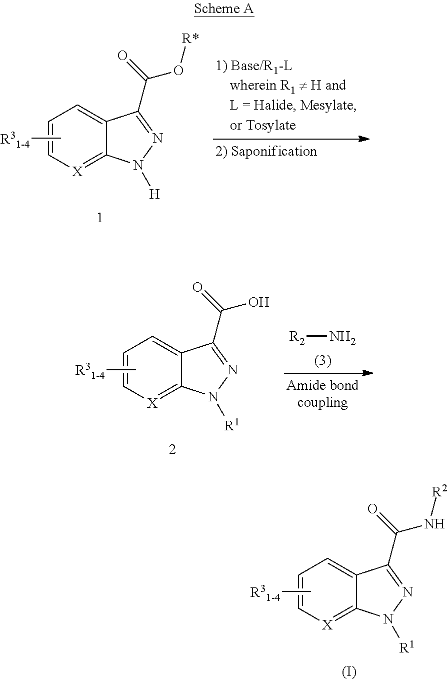

Starting compound 1, wherein X is either carbon or nitrogen and R* is a carboxyl protecting group such as alkyl or aralkyl, can be treated with a base and an alkylating agent. Exemplary bases include sodium hydride, potassium tert-butoxide, sodium hexamethyldisilazide, and potassium carbonate, and exemplary alkylating agents include R¹-L where L is a leaving group, such as a halogen, or a mesylate, or a tosylate, and R¹ is as described in the description of general formula (I). The reaction generally produces a mixture of regioisomers wherein the alkylation occurs either on N1 or N2 position of the indazole ring, depending upon the base and the alkylating agent. The desired N1-alkylated regioisomer is isolated in pure form by either chromatographic separation, or recrystallization of the crude product mixture. Saponification of the alkylated product with an aqueous base such as sodium hydroxide, potassium hydroxide, or lithium hydroxide gives compound 2.

Compound 2 may be coupled with an amine 3 by using reaction conditions well known in the art for peptide bond synthesis [see, for example, Bodanszky and Bodanszky, The Practice of Peptide Chemistry, Springer-Verlag (1984); Bodanszky, Principles of Peptide Synthesis, Springer-Verlag (1984); Han, S-Y and Kim, Y-A, Tetrahedron, vol. 60, pp 2447-2467 (2004)] to give a compound of formula (I). Exemplary reagents for activating the carboxyl group of compound 2 for reacting with the amine 3 include carbodiimide reagents such as N,N′-dicyclohexylcarbodiimide (DCC) and 1-[3-(dimethylamino)propyl]-3-ethylcarbodimide (EDC), either alone or in combination with 1-hydroxybenzotriazole (HOBt), and uronium reagents such as O-(7-azabenzotriazol-1-yl)-1,1,3,3-tetramethyluronium hexafluorophosphate (HATU), O-(benzotriazol-1-yl)-1,1,3,3-tetramethyluronium hexafluorophosphate (HBTU), and O-(benzotriazol-1-yl)-1,1,3,3-tetramethyluronium tetrafluoroborate (TBTU).

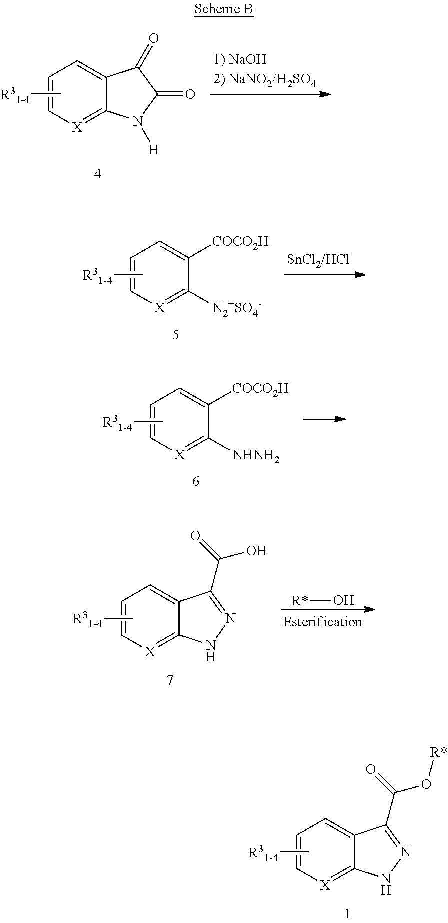

Starting compound 1, wherein X is a carbon and R* is a carboxyl protecting group such as alkyl or aralkyl, can be prepared from compound 4 according to the procedure of Johnson, B. L.; Rodgers, J. D. Syn. Comm. 2005, 35, 2681-2684 as shown in Scheme B. Thus, compound 4 is converted to compound 5 via base-catalyzed ring opening followed by diazotization. Reduction of compound 5 to produce compound 6, and subsequent ring closure gives compound 7. Esterification of compound 7 with a suitable alcohol of the formula R*—OH and an acid catalyst gives compound 1.

Starting compound 1, wherein X is a nitrogen and R* is a carboxyl protecting group such as alkyl or aralkyl, can be prepared according to known methods in the literature [see, for example, Lynch, B. M. et al, Canadian Journal of Chemistry, vol. 66, pp 420-428 (1988); Huang, S. et al, Bioorganic & Medicinal Chemistry Letters, vol. 17, pp 1243-1245 (2007); Lin, R. et al, Bioorganic & Medicinal Chemistry Letters, vol. 17, pp 4297-4302 (2007)].

Amine compounds 3 (R₂—NH₂) are either commercially available, or readily prepared according to methods known in the art as depicted in the protocols for representative Preparations herein.

Compounds of the invention are available by either the methods described herein in the Methods, Examples and Preparations, or suitable adaptations thereof using methods known in the art. It is to be understood that the synthetic transformations mentioned herein may be carried out in various different sequences in order that the desired compounds may be efficiently assembled. The skilled chemist will exercise his judgment and skill as to the most efficient sequence of reactions for synthesis of a given target compound.

The compounds, salts and solvates (including hydrates) of the invention may be separated and purified by conventional methods.

Separation of diastereomers may be achieved by conventional techniques, e.g. by chromatography or HPLC of a stereoisomeric mixture of a compound of formula (I) or a suitable salt or derivative thereof. An individual enantiomer of a compound of formula (I) may also be prepared from a corresponding optically pure intermediate or by resolution, such as by chromatography of the corresponding racemate using a suitable chiral support or by fractional crystallization of the diastereomeric salts formed by a reaction of the corresponding racemate with a suitable optically active acid or base.

### Biological Evaluation

### Method for Assessing Biological Activities:

The Human CB1 receptor binding affinity and other biological activities of the compounds of this invention are determined by the following procedures. Membrane preparation: Human Embryonic Kidney (HEK) Cells expressing the human CB1 receptor under transcriptional regulation of a tetracycline inducible promoter were grown in Dulbecco's Modified Essential Medium with sodium pyruvate (Invitrogen, Carlsbad, Calif.) containing 10% tetracycline free fetal bovine serum (Clonetech, Mountain View, Calif.) 100 μg/ml hygromycin (Calbiochem, San Diego, Calif.), 5 μg/ml blasticidin (Invitrogen). CB1 receptor expression was induced by addition of 1 μg/ml doxycycline (Calbiochem) and incubation for an additional 24 hours. Cells were released from flasks using Cell Dissociation Buffer (Invitrogen). Cells were pelleted by centrifugation at 500×G for 5 minutes. Membranes were prepared by resuspending cells in ice cold TEE Buffer (25 mM Tris pH 7.4, 5 mM EDTA, 5 mM EGTA, Complete Protease Inhibitor (Roche, Basel, Switzerland)). Cells were lysed with 12 strokes of a dounce homogenizer. Unlysed cells were pelleted by centrifugation at 500×G for 5 minutes. Membranes were pelleted by centrifugation at 25,000×G for 30 minutes. Membranes were resuspended in TEE, dounced 12 strokes, and pelleted a second time at 25,000×G for 30 minutes. Membrane pellet was resuspended in 50 mM Tris pH 7.4, 100 mM NaCl, 3 mM MgCl₂, 0.2 mM EGTA, Complete Protease Inhibitor (Roche). Protein concentration was determined using the Micro-BCA Protein Assay Kit (Pierce, Rockford, Ill.) using BSA as a standard. Membranes were quick frozen and stored at −80 degrees Celsius until use.

Binding experiments: 50 μl of test compound was incubated with 50 μl of [³H] CP-55,940 (Perkin Elmer, Boston, Mass.) (final concentration=500 μM) and 150 μl of membrane homogenate (1 μg/well) in polypropylene 96-well plates (Corning, Acton, Mass.). Final reaction conditions were 50 mM Tris pH 7.4, 100 mM NaCl, 3 mM MgCl₂, 0.2 mM EGTA, 0.04% BSA. Nonspecific binding was determined by incubation with 50 μM WIN-55, 212-2 (Tocris, Ellisville, Mo.). After incubation at room temperature for 60 minutes reactions were harvested by vacuum filtration through Unifilter GF/B-96 filters (Perkin Elmer) that had been presoaked in assay buffer containing 0.5% BSA (Sigma, St. Louis, Mo.) using a FilterMate Plate Harvester (Perkin Elmer). Filters were rinsed 4 times with 50 mM Tris pH 7.4, 0.025% Tween-20 and dried at 50 degrees Celsius for at least 30 minutes. 40 μl of Microscint-20 (Perkin Elmer) was added per well, and plates were counted using a Top-Count Microplate Scintillation Counter (Perkin Elmer). Binding data were analyzed and EC₅₀ and K, values calculated using Graph Pad Prism 4.0 Software.

### GTPγS Binding:

Membrane preparation: CHO cells expressing the human CB1 receptor were grown to 80% confluence in Ham's F-12 Nutrient Medium (Invitrogen) containing 10% fetal bovine serum (Invitrogen), 1% pen/strep (Invitogen), 1% Nonessential amino acids (Invitrogen) and 500 μg/ml G418 (Invitrogen). Cells were released from flasks using Cell Dissociation Buffer (Invitrogen). Cells were pelleted by centrifugation at 500×G for 5 minutes. Membranes were prepared by resuspending cells in ice cold Assay Buffer (25 mM Tris pH 7.4, 5 mM EDTA, 5 mM EGTA, Complete Protease Inhibitor (Roche)). Cells were lysed with 12 strokes of a dounce homogenizer. Unlysed cells were pelleted by centrifugation at 500×G for 5 minutes. Membranes were pelleted by centrifugation at 25,000×G for 30 minutes. Membranes were resuspended in TEE, dounced 12 strokes, and pelleted a second time at 25,000×G for 30 minutes. Membrane pellet was resuspended in 50 mM Tris pH 7.4, 100 mM NaCl, 3 mM MgCl₂, 0.2 mM EGTA, Complete Protease Inhibitor (Roche). Protein concentration was determined using the Micro-BCA Protein Assay Kit (Pierce) using BSA as a standard. Membranes were frozen and stored at −80 degrees Celsius until use.

GTPγS Binding: 40 μl of test compound was incubated with 20 μl of [³⁵S] GTPγS (Perkin Elmer) (1250 Ci/millimole) and 140 μl of membrane homogenate (5 μg/well) in polypropylene 96-well plates (Corning). Final reaction conditions were 50 mM Tris pH 7.4, 100 mM NaCl, 3 mM MgCl₂, 0.2 mM EGTA, 0.04% BSA. After incubation at 37 degrees Celsius for 45 minutes reactions were harvested by vacuum filtration through Unifilter GF/B-96 filters (Perkin Elmer) using a FilterMate Plate Harvester (Perkin Elmer). Filters were rinsed 4 times with ice cold 50 mM Tris pH 7.4, 3 mM MgCl₂, 0.2 mM EGTA and dried at 50 degrees Celsius for at least 30 minutes. 40 μl of Microscint-20 (Perkin Elmer) was added per well, and plates were counted using a Top-Count Microplate Scintillation Counter (Perkin Elmer). Binding data were analyzed and EC₅₀ values were calculated using Graph Pad Prism 4.0 Software.

The above protocol assays were used to determine biological activity. The Ki towards human CB1 receptors for certain compounds of the invention are measured to be 0.01-1000 nM. The EC50 towards human CB1 receptors in the GTPγS assay for certain compounds of the invention are measured to be 0.1-5000 nM. Table 1 shows certain biological activities for some of the exemplified compounds.

TABLE 1
CB1 Binding Affinity and Agonism

| Example No. | CB1 Ki (nM) | GTPγS EC50 (nM) |
|---|---|---|
| 1 | 0.36 | 0.98 |
| 2 | 0.9 | 23.2 |
| 3 | 49.9 | 298 |
| 4 | 708 | ND* |
| 5 | 954 | ND* |
| 6 | 12.6 | 160 |
| 7 | 2.04 | 12.9 |
| 8 | 118 | 209 |
| 9 | 84.2 | ND* |
| 10 | 1.91 | 37.5 |
| 11 | 0.29 | 0.55 |
| 12 | 11.5 | 302 |
| 13 | 0.73 | 11.9 |
| 17 | 4.69 | 149 |
| 19 | 2.57 | 20.5 |
| 20 | 51.1 | 216 |
| 27 | 0.33 | 14.7 |
| 28 | 2.05 | 121 |
| 30 | 9.22 | 78.9 |
| 33 | 0.24 | 0.92 |
| 34 | 154 | ND* |
| 35 | 35.3 | 271 |
| 38 | 0.14 | 2.42 |
| 43 | 27.1 | 101 |
| 45 | 8.79 | 21.4 |
| 65 | 3.85 | 90 |
| 67 | 46.5 | 827 |
| 68 | 4.61 | 90.1 |
| 69 | 18.8 | 183 |
| 71 | 8.85 | 314 |
| 73 | 22.9 | 217 |
| 77 | 5.39 | 48.1 |
| 78 | 0.59 | 2.88 |
| 79 | 2.02 | 27.1 |
| 80 | 0.21 | 1.82 |
| 81 | 0.32 | 0.98 |
| 82 | 1.12 | 22.1 |
| 83 | 15.3 | 720 |
| 85 | 1.72 | 16.1 |
| 86 | 2.75 | 34.2 |
| 87 | 2.26 | 46.1 |
| 88 | 15.4 | 132 |
| 89 | 63.4 | 539 |
| 90 | 27.4 | 385 |
| 91 | 1.87 | 53.8 |
| 92 | 14.1 | 265 |
| 93 | 14.3 | 41.8 |
| 94 | 27.5 | 77 |
| 95 | 2.22 | 13.7 |
| 96 | 1.18 | 16.9 |
| 97 | 1.04 | 16.7 |
| 98 | 0.98 | 8.63 |
| 99 | 0.18 | 0.5 |
| 103 | 2.68 | 9.08 |
| 108 | 3.78 | 27 |
| 109 | 8.14 | 110 |
| 110 | 28.9 | 237 |
| 111 | 0.72 | 9.73 |
| 112 | 0.51 | 31.8 |
| 113 | 7.79 | 188 |
| 115 | 1.09 | 8.72 |
| 116 | 13.5 | 49.6 |
| 117 | 9.54 | 168 |
| 118 | 0.7 | 23.8 |
| 120 | 3.05 | 40 |
| 122 | 0.73 | 13.1 |
| 126 | 0.97 | 2.55 |
| 127 | 32.8 | 136 |
| 128 | 0.97 | 3.58 |
| 129 | 12.6 | 106 |
| 130 | 14.7 | 72.7 |
| 131 | 0.6 | 13.2 |
| 133 | 0.55 | 6.35 |
| 134 | 32.7 | 326 |
| 135 | 3.47 | 14.3 |
| 136 | 10.7 | 115 |
| 137 | 0.69 | 1.69 |
| 139 | 0.82 | 6.36 |
| 140 | 39.3 | 645 |
| 141 | 9.42 | 41.1 |
| 148 | 1.8 | 32.2 |
| 151 | 1.63 | 6.3 |
| 154 | 0.53 | 4.81 |
| 160 | 1.45 | 32.3 |
| 163 | 4.45 | 180 |
| 166 | 5.14 | 132 |
| 170 | 0.27 | 0.7 |
| 171 | 0.42 | 0.44 |
| 172 | 0.42 | 0.42 |
| 173 | 2.37 | 5.12 |
| 174 | 1.1 | 1.81 |
| 175 | 0.19 | 0.64 |
| 176 | 0.22 | 0.51 |
| 177 | 0.28 | 0.31 |
| 178 | 0.56 | 1.61 |
| 179 | 0.87 | 5.41 |
| 180 | 0.37 | 3.81 |
| 181 | 0.1 | 0.33 |
| 182 | 0.34 | 2.23 |
| 183 | 0.26 | 0.83 |
| 184 | 0.37 | 3.94 |
| 185 | 0.51 | 10.2 |
| 186 | 0.19 | 1.03 |
| 187 | 0.09 | 1.09 |
| 198 | 1.19 | 7.07 |
| 199 | 1.32 | 8.94 |
| 200 | 4.8 | 35 |
| 201 | 14.2 | 70.1 |
| 202 | 0.8 | 3.07 |
| 203 | 10.2 | 63.2 |
| 211 | 3.08 | 18.3 |
| 212 | 52.1 | |
| 213 | 15.7 | 57.7 |
| 214 | 3.87 | 23.4 |
| 215 | 7.69 | 41.3 |
| 216 | 225 | |
| 217 | >400 | |
| 218 | 7.92 | 360 |
| 219 | >400 | |
| 220 | 1.26 | 3.78 |
| 221 | 87.5 | |
| 222 | 21.4 | |
| 223 | 1.12 | 4.76 |
| 224 | 6.77 | 19.3 |
| 225 | 6.3 | 26.9 |
| 226 | 0.18 | 0.73 |
| 227 | >400 | |
| 228 | 4.26 | 15.1 |
| 229 | 31.3 | |
| 230 | 6.5 | 31.4 |
| 231 | 2.25 | 5.12 |
| 232 | 54.2 | |
| 233 | 2.45 | 11.5 |
| 234 | 13.4 | 36.1 |
| 235 | 222 | |
| 236 | 0.94 | 3.91 |
| 237 | >400 | |
| 238 | 6.46 | 25.7 |
| 239 | 46.8 | |
| 240 | 152 | |
| 241 | 1.65 | 5.72 |
| 242 | 0.36 | 3.37 |
| 243 | 11.3 | 91.3 |
| 244 | 2.42 | 16.2 |
| 245 | 2.61 | 12.4 |
| 246 | 6.58 | 69 |
| 247 | 0.65 | 0.95 |
| 248 | 108 | |
| 249 | 2.51 | 16.3 |
| 250 | 3.72 | 18.1 |
| 251 | 0.51 | 2.33 |
| 252 | 205 | |
| 253 | 4.5 | 26 |
| 254 | 12.3 | 153 |
| 255 | 13.1 | 130 |
| 256 | 98.6 | |
| 257 | 224 | |
| 258 | >400 | |
| 259 | 132 | |
| 260 | >400 | |
| 261 | 76.7 | |
| 262 | 8.25 | 38.9 |
| 263 | 8.36 | 100 |
| 264 | 6.75 | 103 |
| 265 | 13.1 | 82 |
| 266 | 0.94 | 4.34 |
| 267 | 78 | |
| 268 | >400 | |
| 269 | 23.8 | |
| 270 | 0.76 | 2.62 |
| 271 | 2.91 | 24.9 |
| 272 | >400 | |
| 273 | >400 | |
| 274 | >400 | |
| 275 | >400 | |
| 276 | >400 | |
| 277 | 31.7 | |
| 278 | 68.8 | |
| 279 | 54.1 | |
| 280 | 176 | |
| 281 | 4.83 | 37.4 |
| 282 | 0.17 | 0.78 |
| 283 | >400 | |
| 284 | 1.03 | 12.3 |
| 285 | 27.9 | |
| 286 | 5.74 | 36.1 |
| 287 | >400 | |
| 288 | 1.18 | 9.53 |
| 289 | 5.13 | 35.8 |
| 290 | 92 | |
| 291 | 1.2 | |
| 292 | 5.25 | 19 |
| 293 | >400 | |
| 294 | >400 | |
| 295 | 9.17 | 64.3 |
| 296 | 64.1 | |
| 297 | 124 | |
| 298 | 182 | |
| 299 | 8.56 | 23.8 |
| 300 | 5.85 | 121 |
| 301 | 70.3 | |
| 302 | 5.41 | 33.1 |
| 303 | 2.27 | 11 |
| 304 | 152 | |
| 305 | 18 | 86.4 |
| 306 | 0.78 | 1.39 |
| 307 | 1.27 | 1.56 |
| 308 | 2.63 | 5.55 |
| 309 | 1.59 | 2.59 |
| 310 | 1.48 | 2.1 |
| 311 | 147 | |
| 312 | 178 | |
| 313 | 273 | |
| 314 | 130 | |
| 315 | 2.91 | 8.67 |
| 316 | 243 | |
| 317 | 31.1 | |
| 318 | 68.7 | |
| 319 | 45.8 | |
| 320 | 12 | 63.2 |
| 321 | 1.58 | 16.6 |
| 322 | 8.89 | 109 |
| 323 | 2.99 | 22.5 |
| 324 | 0.15 | 2.6 |
| 325 | 1.97 | 5.33 |
| 326 | 15.8 | 53.2 |
| 327 | 4.19 | 18.1 |
| 328 | 0.71 | 1.7 |
| 329 | 2.93 | 8 |
| 330 | 0.2 | 0.41 |
| 331 | 2.2 | 9.9 |
| 332 | 15 | 27.3 |
| 333 | 1.49 | 3.75 |
| 334 | 1.72 | 9.41 |
| 335 | 3.21 | 14.9 |
| 336 | 0.11 | 0.52 |
| 337 | 3.48 | 21.1 |
| 338 | 3.43 | 24.9 |
| 339 | 5.36 | 21.6 |
| 340 | 2.59 | 7.22 |
| 341 | 3.74 | 13.9 |
| 342 | 20.5 | |
| 343 | 216 | |
| 344 | 10.1 | 60.2 |
| 345 | 0.61 | 1.69 |
| 346 | 5.14 | 12.1 |
| 347 | 24.4 | |
| 348 | 7.83 | 19.7 |
| 349 | 101 | |
| 350 | 229 | |
| 351 | 24.3 | |
| 352 | 4.14 | 49.5 |
| 353 | 72.8 | |
| 354 | 11.7 | >500 |
| 355 | 52.9 | |
| 356 | 32.6 | |
| 357 | 2.93 | 48.6 |
| 358 | 4.89 | 7.46 |
| 359 | 47.2 | |
| 360 | 73.5 | |
| 361 | 41.6 | |
| 362 | 125 | |
| 363 | 57.8 | |
| 364 | 20.9 | |
| 365 | 11.9 | 34.8 |
| 366 | 241 | |
| 367 | 41.4 | |
| 368 | 2 | 4.55 |
| 369 | 28.1 | |
| 370 | 132 | |
| 371 | 54.8 | |
| 372 | 22.6 | |
| 373 | 14.6 | 11.6 |
| 374 | 7.33 | 12.5 |
| 375 | 7.92 | 31.4 |
| 376 | 1.52 | 4.4 |
| 377 | 22.5 | |
| 378 | 158 | |
| 379 | >400 | |
| 380 | >400 | |
| 381 | 15.3 | 26.9 |
| 382 | 238 | |
| 383 | >400 | |
| 384 | 286 | |
| 385 | 166 | |
| 386 | 209 | |
| 387 | >400 | |
| 388 | >400 | |
| 389 | >400 | |
| 390 | >400 | |
| 391 | >400 | |
| 392 | >400 | |
| 393 | >400 | |
| 394 | >400 | |
| 395 | 41.2 | 30.8 |
| 396 | 239 | |
| 397 | 243 | |
| 398 | 5.8 | 26.6 |
| 399 | >400 | |
| 400 | 12.3 | 28.1 |
| 401 | >400 | |
| 402 | 277 | |
| 403 | >400 | |
| 404 | 13.1 | 38.2 |
| 405 | 48.1 | |
| 406 | 89.7 | |
| 407 | 36.2 | |
| 408 | >400 | |
| 409 | 73 | |
| 410 | 104 | |
| 411 | 3.73 | 21.3 |
| 412 | >400 | |
| 413 | 14 | 52 |
| 414 | 7.61 | 38.6 |
| 415 | 8.69 | 10.8 |
| 416 | 9.26 | 47.1 |
| 417 | 7.84 | 25.7 |
| 418 | 0.78 | 4.07 |
| 419 | 110 | |
| 420 | 11.2 | 43.7 |
| 421 | 2.88 | 17.2 |
| 422 | 4.67 | 19.6 |
| 423 | 5.19 | 30.8 |
| 424 | 1.28 | 10.2 |
| 425 | 0.92 | 3.36 |
| 426 | 90 | |
| 427 | 15 | 50.5 |
| 428 | 0.89 | |
| 429 | 0.44 | |
| 430 | 18.2 | |
| 431 | 13.6 | |
| 432 | 15.7 | 101 |
| 433 | 35.5 | |
| 434 | 55.1 | |
| 435 | 6.5 | |
| 436 | 1.13 | |
| 437 | 2.79 | |
| 438 | 10.9 | 20 |
| 439 | 3.26 | |
| 440 | 104 | |
| 441 | >400 | |
| 442 | >400 | |
| 443 | >400 | |
| 444 | >400 | |
| 445 | >400 | |
| 446 | 168 | |
| 447 | 170 | |
| 448 | >400 | |
| 449 | >400 | |
| 450 | 241 | |
| 451 | >400 | |
| 452 | >400 | |
| 453 | >400 | |
| 454 | >400 | |
| 455 | 33.6 | |
| 456 | 43.8 | |
| 457 | 1.77 | 108 |
| 458 | 120 | |
| 459 | 2.78 | 17.7 |
| 460 | 24.6 | |
| 461 | >400 | |
| 462 | 2.29 | 13 |
| 463 | 274 | |
| 464 | 58 | |
| 465 | >400 | |
| 466 | >400 | |
| 467 | 53.8 | |
| 468 | 23.5 | |
| 469 | 80.7 | |
| 470 | 11.2 | 33.7 |
| 471 | >400 | |
| 472 | 19.6 | 52.7 |
| 473 | 17 | 41.8 |
| 474 | 41.2 | |
| 475 | 141 | |
| 476 | 6.48 | 31.4 |
| 477 | 28.3 | |
| 478 | 21.3 | 23.7 |
| 479 | 13.4 | 131 |
| 480 | 15.3 | 42.6 |
| 481 | 52.6 | |
| 482 | 12.1 | 22.2 |
| 483 | 84 | |
| 484 | >400 | |
| 485 | 152 | |
| 486 | 43.9 | |
| 487 | 109 | |
| 488 | 5.61 | 21.4 |
| 489 | 127 | |
| 490 | 12.4 | 63.1 |
| 491 | 88.5 | |
| 492 | 1.32 | 9.71 |
| 493 | 0.95 | 9.94 |
| 494 | 0.34 | 1.17 |
| 495 | 130 | |
| 496 | 16.5 | 30.2 |
| 497 | 16.7 | 59.5 |
| 498 | 16.4 | 38.3 |
| 499 | 18.3 | 204 |
| 500 | 10.1 | 47.4 |
| 501 | 24.2 | 16.8 |
| 502 | 17.3 | 36.9 |
| 503 | 321 | |
| 504 | 21.3 | 132 |
| 505 | 301 | |
| 506 | 1.3 | 8.97 |
| 507 | 212 | |
| 508 | 2.71 | 16.2 |
| 509 | 0.45 | 7.55 |
| 510 | 6.87 | 24.8 |
| 511 | 0.68 | 6.7 |
| 512 | 8.4 | 31.4 |
| 513 | 2.3 | 13.7 |
| 514 | 3.03 | 33.3 |
| 515 | 37.5 | |
| 516 | 4.28 | 44.6 |
| 517 | 15.9 | 111 |
| 518 | 1.8 | 13.6 |
| 519 | 0.95 | 5.77 |
| 520 | 1.88 | 10.1 |
| 521 | >400 | |
| 522 | >400 | |
| 523 | 5.22 | 2.8 |

*ND = Not determined

### Examples and Preparations

The invention is illustrated in the following non-limiting examples and preparations in which, unless stated otherwise: all operations were carried out at room or ambient temperature, that is, in the range of 18-25 degrees Celsius; evaporation of solvent was carried out using a rotary evaporator under reduced pressure with a bath temperature of up to 60 degrees Celsius; reactions were monitored by thin layer chromatography (TLC) and reaction times are given for illustration only; melting points (mp) given are uncorrected (polymorphism may result in different melting points); the structure and purity of all isolated compounds were assured by at least one of the following techniques: TLC (Merck silica gel 60 F₂₅₄ precoated TLC plates or Merck NH₂ gel (an amine coated silica gel) F₂₅₄ₛ precoated TLC plates), mass spectrometry, nuclear magnetic resonance spectra (NMR), infrared absorption spectra (IR) or microanalysis. Yields are given for illustrative purposes only. Workup with a cation-exchange column was carried out using SCX cartridge (Varian BondElute), which was preconditioned with methanol. Flash column chromatography was carried out using Merck silica gel 60 (63-200 □m), Wako silica gel 300HG (40-60 □m), Fuji Silysia NH gel (an amine coated silica gel) (30-50 □m), Biotage KP-SIL (32-63 □m) or Biotage AMINOSILICA (an amine coated silica gel) (40-75 □m). Preparative TLC was carried out using Merck silica gel 60 F₂₅₄ precoated TLC plates (0.5 or 1.0 mm thickness). Low-resolution mass spectral data (EI) were obtained on an Integrity (Waters) mass spectrometer. Low-resolution mass spectral data (ESI) were obtained on ZMD™ or ZQ™ (Waters) and mass spectrometer. NMR data were determined at 270 MHz (JEOL JNM-LA 270 spectrometer), 300 MHz (JEOL JNM-LA300 spectrometer) or 600 MHz (Bruker AVANCE 600 spectrometer) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, quint=quintet, m=multiplet, bs=broad singlet, etc. IR spectra were measured by a Fourier transform infrared spectrophotometer (Shimazu FTIR-8300). Chemical symbols have their usual meanings; by (boiling point), mp (melting point), rt (room temperature), L (liter(s)), mL (milliliter(s)), g (gram(s)), mg (milligram(s)), mol (moles), mmol (millimoles), eq. (equivalent(s)), quant. (quantitative yield). Following abbreviations may be used in examples: CDI (N,N′-carbonyldiimidazole), DMF (N,N-dimethylformamide), DMSO (dimethylsulfoxide), EDC.HCl (1-ethyl-3-(3-dimethylaminopropyl)carbodiimide hydrochloride), HATU [2-(7-aza-1H-benzotriazol-1-yl)-1,1,3,3-tetramethyluronium hexafluorophosphate], TBTU [2-(1H-benzotriazol-1-yl)-1,1,3,3-tetramethyluronium tetrafluoroborate], EtOH (ethanol), HOBt (1-Hydroxy-1H-benzotriazole), MeOH (methanol), THF (tetrahydrofuran), and TFA (trifluoroacetic acid). Rf means retention time measured by LC/MS (Waters 2790) under the following condition;

Column: Xterra, C18, 5 μm, 4.6×50 mm (40 degrees Celsius)

flow: 2.0 mL/min

Gradient: Water/MeOH/1% HCO₂H aq.=90/5/5 to 0/95/5

Total run time: 2.5 minutes.

### Example 1

### N-[(1S)-1-(Aminocarbonyl)-2,2-dimethylpropyl]-1-(4-fluorobenzyl)-1H-indazole-3-carboxamide

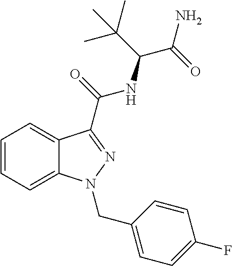

### Step 1: Methyl 1-(4-fluorobenzyl)-1H-indazole-3-carboxylate

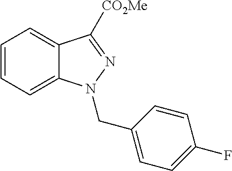

To a solution of methyl indazole-3-carboxylate (1.0 g, 5.67 mmol) in anhydrous THF (30 ml), cooled in an ice bath was added slowly solid potassium tert-butoxide (694 mg, 6.18 mmol). The mixture was then stirred at room temperature for 1 h, followed by the addition of 4-fluorobenzyl bromide (1.1 ml, 8.96 mmol) at 0° C. The reaction mixture was stirred for 5 h at room temperature, then quenched by the addition of water and extracted with ethyl acetate. The organic layer was dried over sodium sulfate and concentrated under reduced pressure. The residue was purified by column chromatography over silica gel (100-200 mesh) using 15% ethyl acetate-hexane as eluant to afford pure product methyl 1-(4-fluorobenzyl)-1H-indazole-3-carboxylate (1.5 g, yield 92%).

¹H NMR (400 MHz, CDCl₃) δ: 4.04 (s, 3H), 5.66 (s, 2H), 6.95-7.00 (m, 2H), 7.18-7.22 (m, 2H), 7.28-7.39 (m, 3H), 8.22-8.24 (m, 1H). FIA-MS: 285.2 [M+H]⁺, 307.2 [M+H+Na]⁺.

### Step 2: 1-(4-Fluorobenzyl)-1H-indazole-3-carboxylic acid

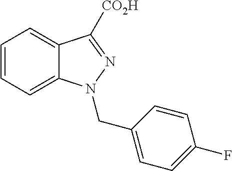

To a solution of 1-(4-fluorobenzyl)-1H-indazole-3-carboxylic acid methyl ester (300 mg, 1.05 mmol), dissolved in methanol was added 1M NaOH (2 mL). The mixture was stirred for 12 h at ambient temperature. After completion of the reaction, mixture was evaporated up to dryness. The residue was dissolved in water and neutralized with 1N HCl and extracted with ethyl acetate. The organic layer was dried over sodium sulfate and concentrated to afford desired product 1-(4-fluorobenzyl)-1H-indazole-3-carboxylic acid as white solid (280 mg, yield 98%).

¹H NMR (400 MHz, DMSO-d₆) δ: 5.76 (s, 2H), 7.14-7.18 (m, 2H), 7.29-7.35 (m, 3H), 7.45-7.49 (m, 1H), 7.85 (d, J=8.4 Hz, 1H), 8.09 (d, J=8.0 Hz, 1H), 13.1 (br s, 1H). FIA-MS: 271.3 [M+H]⁺, 293.3 [M+H+Na]⁺.

### Step 3: N-[(1S)-1-(aminocarbonyl)-2,2-dimethylpropyl]-1-(4-fluorobenzyl)-1H-indazole-3-carboxamide

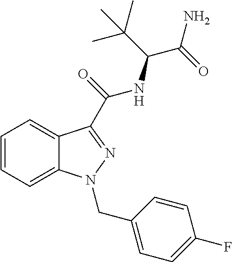

A mixture of 1-(4-fluorobenzyl)-1H-indazole-3-carboxylic acid (100 mg, 0.37 mmol), L-tert-leucinamide (Preparation 1, 73.5 mg, 0.56 mmol), EDC.HCl (108 mg, 0.56 mmol), HOBt (76 mg, 0.56 mmol) and N,N-diisopropylethylamine (0.33 mL, 1.88 mmol) in dry DMF (5 mL) was stirred at room temperature for 18 h. Then after completion of the reaction, water was added to the reaction mixture and extracted with ethyl acetate. The organic layer was separated, dried over sodium sulfate and concentrated under reduced pressure to give crude material, which on column chromatography over silica gel (100-200 mesh) using 50% ethyl acetate-hexane as eluant to afford pure product N-[(1S)-1-(aminocarbonyl)-2,2-dimethylpropyl]-1-(4-fluorobenzyl)-1H-indazole-3-carboxamide as white solid (70 mg, yield 49%).

¹H NMR (400 MHz, CD₃OD) δ: 1.10 (s, 9H), 4.53 (s, 1H), 5.71 (s, 2H), 7.02-7.06 (m, 2H), 7.26-7.32 (m, 3H), 7.40-7.44 (m, 1H), 7.59 (d, J=8.8 Hz, 1H), 8.21 (d, J=8.0 Hz, 1H). FIA-MS: 383.2 [M+H]⁺, 405.1 [M+H+Na]⁺.

### Example 2

### N-[(1S)-1-(aminocarbonyl)-2-methylpropyl]-1-(4-fluorobenzyl)-1H-indazole-3-carboxamide

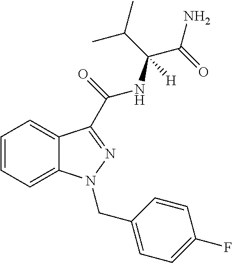

A mixture of 1-(4-fluorobenzyl)-1H-indazole-3-carboxylic acid (Example 1, Step 2, 100 mg, 0.37 mmol), L-valinamide (65.5 mg, 0.56 mmol), EDC.HCl (108 mg, 0.56 mmol), HOBt (76 mg, 0.56 mmol) and N,N-diisopropylethylamine (0.33 mL, 1.88 mmol) in dry DMF (5 mL) was stirred at room temperature for 18 h. Then after completion of the reaction, water was added to the reaction mixture and extracted with ethyl acetate. The organic layer was separated, dried over sodium sulfate and concentrated under reduced pressure to give crude material, which was purified by column chromatography over silica gel using 50% ethyl acetate-hexane as eluant to afford N-[(1S)-1-(aminocarbonyl)-2-methylpropyl]-1-(4-fluorobenzyl)-1H-indazole-3-carboxamide as white solid (88 mg, yield 64%)

¹H NMR (400 MHz, CD₃OD) δ: 1.03 (d, J=6.8 Hz, 3H), 1.05 (d, J=6.8 Hz, 3H), 2.14-2.24 (m, 1H), 4.50 (d, J=6.4 Hz, 1H), 5.71 (s, 2H), 7.02-7.06 (m, 2H), 7.26-7.32 (m, 3H), 7.40-7.44 (m, 1H), 7.59 (d, J=8.8 Hz, 1H), 8.21 (d, J=8.0 Hz, 1H). FIA-MS: 369.2 [M+H]⁺, 391.3 [M+H+Na]⁺.

### Example 3

### N-[(1S)-2-amino-2-oxo-1-phenylethyl]-1-(4-fluorobenzyl)-1H-indazole-3-carboxamide

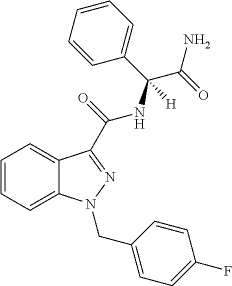

A mixture of 1-(4-fluorobenzyl)-1H-indazole-3-carboxylic acid (Example 1, Step 2, 100 mg, 0.37 mmol), (S)-2-amino-2-phenyl-acetamide (84.7 mg, 0.56 mmol), EDC.HCl (108 mg, 0.56 mmol), HOBt (76 mg, 0.56 mmol) and N,N-diisopropylethylamine (0.33 mL, 1.88 mmol) in dry DMF (5 mL) was stirred at room temperature for 18 h. Then after completion of the reaction, water was added to the reaction mixture and extracted with ethyl acetate. The organic layer was separated, dried over sodium sulfate and concentrated under reduced pressure to give crude material, which was purified by column chromatography over silica gelusing 50% ethyl acetate-hexane as eluant to afford N-[(1S)-2-amino-2-oxo-1-phenylethyl]-1-(4-fluorobenzyl)-1H-indazole-3-carboxamide as white solid (90 mg, yield 60%).

¹H NMR (400 MHz, CD₃OD) δ: 5.68 (s, 1H), 5.70 (s, 2H), 7.01-7.05 (m, 2H), 7.24-7.43 (m, 7H), 7.53-7.59 (m, 3H), 8.18 (d, J=8.4 Hz, 1H). FIA-MS: 403.3 [M+H]⁺, 425.1 [M+H+Na]⁺.

### Example 4

### N-α-{[1-(4-fluorobenzyl)-1H-indazol-3-yl]carbonyl}-L-phenylalaninamide

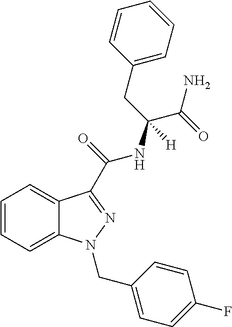

A mixture of 1-(4-fluorobenzyl)-1H-indazole-3-carboxylic acid (Example 1, Step 2, 100 mg, 0.37 mmol), L-phenylalaninamide (92 mg, 0.56 mmol), EDC.HCl (108 mg, 0.56 mmol), HOBt (76 mg, 0.56 mmol) and N,N-diisopropylethylamine (0.33 mL, 1.88 mmol) in dry DMF (5 mL) was stirred at room temperature for 18 h. Then after completion of the reaction, water was added to the reaction mixture and extracted with ethyl acetate. The organic layer was separated, dried over sodium sulfate and concentrated under reduced pressure to give crude material, which was purified by column chromatography over silica gel using 50% ethyl acetate-hexane as eluant to afford N-α-{[1-(4-fluorobenzyl)-1H-indazol-3-yl]carbonyl}-L-phenylalaninamide as white solid (55 mg, yield 32%).

¹H NMR (400 MHz, CD₃OD) δ: 3.08-3.26 (m, 3H), 5.67 (s, 2H), 7.02-7.06 (m, 2H), 7.17-7.30 (m, 8H), 7.38-7.42 (m, 1H), 7.58 (d, J=8.8 Hz, 1H), 8.14 (d, J=8.4 Hz, 1H). FIA-MS: 417.2 [M+H]⁺.

### Example 5

### N-[(1S)-1-(aminocarbonyl)-2,2-dimethylpropyl]-1-[(5-methylisoxazol-3-yl)methyl]-1H-indazole-3-carboxamide

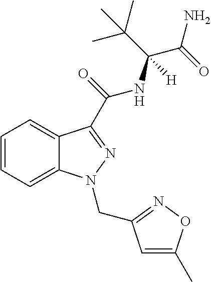

### Step 1: Methyl 1-[(5-methylisoxazol-3-yl)methyl]-1H-indazole-3-carboxylate

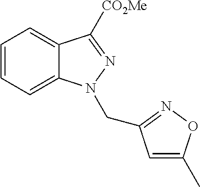

To a solution of methyl indazole-3-carboxylate (200 mg, 1.14 mmol) in anhydrous THF (6 ml), cooled in an ice bath was added slowly potassium tert-butoxide (138.8 mg, 1.23 mmol). The mixture was stirred at room temperature for 1 hr, then 3-chloromethyl-5-methylisoxazole (235 mg, 1.79 mmol) was added at 0° C. This reaction mixture was stirred for 12 h at room temperature. The reaction was quenched by the addition of water and extracted with ethyl acetate. The organic layer was dried over sodium sulfate and concentrated under reduced pressure. The residue was purified by column chromatography over silica gel using 15% ethyl acetate-hexane as eluant to afford methyl 1-[(5-methylisoxazol-3-yl)methyl]-1H-indazole-3-carboxylate (150 mg, yield 42%).

¹H NMR (400 MHz, CDCl₃) δ: 2.32 (s, 3H), 4.05 (s, 3H), 5.70 (s, 2H), 5.84 (s, 1H), 7.30-7.34 (m, 1H), 7.41-7.45 (m, 1H), 7.53 (d, J=8.4 Hz, 1H), 8.20-8.22 (m, 1H). FIA-MS: 272.3 [M+H]⁺, 294.1 [M+H+Na]⁺.

### Step 2: 1-[(5-Methylisoxazol-3-yl)methyl]-1H-indazole-3-carboxylic acid

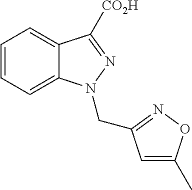

To a solution of methyl 1-[(5-methylisoxazol-3-yl)methyl]-1H-indazole-3-carboxylate (500 mg, 1.84 mmol) in methanol (3 mL) was added 1M NaOH (3 mL). The mixture was stirred for 4 h at ambient temperature. After completion of the reaction, mixture was evaporated up to dryness. The residue was dissolved in water and acidified to pH 6 with 1N HCl and extracted with ethyl acetate. The organic layer was dried over sodium sulfate and concentrated to afford 1-[(5-methylisoxazol-3-yl)methyl]-1H-indazole-3-carboxylic acid as white solid (450 mg, yield 95%).

¹H NMR (400 MHz, DMSO-d₆) δ: 2.32 (s, 3H), 5.83 (s, 2H), 6.05 (s, 1H), 7.34 (t, J=7.6 Hz, 1H), 7.48-7.83 (m, 1H), 7.82 (d, J=8.4 Hz, 1H), 8.09 (d, J=8.0 Hz, 1H), 13.1 (br s, 1H). FIA-MS: 258.3 [M+H]⁺, 280.2 [M+H+Na]⁺.

### Step 3: N-[(1S)-1-(aminocarbonyl)-2,2-dimethylpropyl]-1-[(5-methylisoxazol-3-yl)methyl]-1H-indazole-3-carboxamide

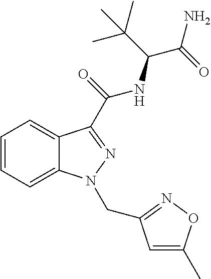

A mixture of 1-[(5-methylisoxazol-3-yl)methyl]-1H-indazole-3-carboxylic acid (100 mg, 0.39 mmol), L-tert-leucinamide (Preparation 1, 77.48 mg, 0.59 mmol), EDC.HCl (114.25 mg, 0.59 mmol), HOBt (80.5 mg, 0.59 mmol) and N,N-diisopropylethylamine (0.35 mL, 2.01 mmol) in dry DMF (5 mL) was stirred at room temperature for 18 h. Then after completion of the reaction, water was added to the reaction mixture and extracted with ethyl acetate. The organic layer was separated, dried over sodium sulfate and concentrated under reduced pressure to give crude material, which was purified by column chromatography over silica gel (100-200 mesh) using 70% ethyl acetate-hexane as eluant to afford N-[(1S)-1-(aminocarbonyl)-2,2-dimethylpropyl]-1-[(5-methylisoxazol-3-yl)methyl]-1H-indazole-3-carboxamide as white solid (45 mg, yield 30%).

¹H NMR (400 MHz, CD₃OD) δ: 1.09 (s, 9H), 2.34 (s, 3H), 4.52-4.54 (m, 1H), 5.75 (s, 2H), 6.01 (s, 1H), 7.28-7.32 (m, 1H), 7.45-7.48 (m, 1H), 7.65 (d, J=8.8 Hz, 1H), 8.22 (d, J=8.0 Hz, 1H). FIA-MS: 370.4 [M+H]⁺, 392.3 [M+H+Na]⁺.

### Example 6

### N-[(1S)-1-(aminocarbonyl)-2,2-dimethylpropyl]-1-(pyridin-2-ylmethyl)-1H-indazole-3-carboxamide

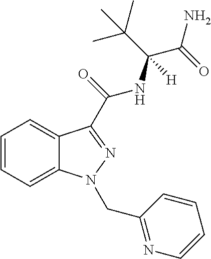

### Step 1: Methyl 1-(pyridin-2-ylmethyl)-1H-indazole-3-carboxylate

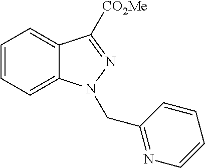

To a solution of methyl indazole-3-carboxylate (200 mg, 1.14 mmol) in anhydrous THF (6 ml), cooled in an ice bath was added slowly solid sodium hydride (840 mg, 7.5 mmol). The mixture was stirred at rt for 2 h, then a solution of 2-(chloromethyl)pyridine hydrochloride (294 mg, 1.79 mmol) in DMF (1 mL) and 1 mL triethylamine were added at 0° C. This reaction mixture was stirred for 12 h at room temperature and then 12 h at 60° C. The reaction was quenched by the addition of water and extracted with ethyl acetate. The organic layer was dried over sodium sulfate and concentrated under reduced pressure, and the residue was purified by column chromatography over silica gel (100-200 mesh) using 15% ethyl acetate-hexane as eluant to afford methyl 1-(pyridin-2-ylmethyl)-1H-indazole-3-carboxylate (100 mg, yield 33%).

¹H NMR (400 MHz, DMSO-d₆) δ: 3.91 (s, 3H), 5.89 (s, 2H), 7.17 (d, J=8.0 Hz, 1H), 7.29-7.38 (m, 2H), 7.49 (t, J=7.2 Hz, 1H), 7.74-7.83 (m, 2H), 8.10 (d, J=8.0 Hz, 1H), 8.47 (br s, 1H). MS 268.1 [M+H]⁺.

### Step 2: 1-(Pyridin-2-ylmethyl)-1H-indazole-3-carboxylic acid

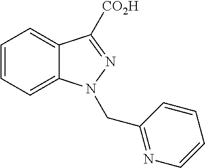

To a solution of methyl 1-pyridin-2-ylmethyl-1H-indazole-3-carboxylate (350 mg, 1.31 mmol) in methanol was added 1M NaOH (3 ml). The mixture was stirred for 6 h at ambient temperature. After completion of the reaction, mixture was evaporated to dryness. The residue was dissolved in water and adjusted the pH to 6 with 1N HCl and extracted with ethyl acetate. The organic layer was dried over sodium sulfate and concentrated to afford desired product 1-pyridin-2-ylmethyl-1H-indazole-3-carboxylic acid as yellowish solid (150 mg, yield 45%).

¹H NMR (400 MHz, DMSO-d₆) δ: 5.87 (s, 2H), 7.15 (d, J=8.0 Hz, 1H), 7.29-7.34 (m, 2H), 7.46 (t, J=7.6 Hz, 1H), 7.74-7.79 (m, 2H), 8.10 (d, J=8.4 Hz, 1H), 8.48 (d, J=4.4 Hz, 1H), 13.1 (br s, 1H). FIA-MS: 254.3 [M+H]⁺, 276.2 [M+H+Na]⁺.

### Step 3: N-[(1S)-1-(aminocarbonyl)-2,2-dimethylpropyl]-1-(pyridin-2-ylmethyl)-1H-indazole-3-carboxamide

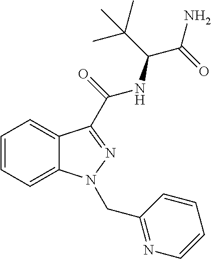

A mixture of 1-(pyridin-2-ylmethyl)-1H-indazole-3-carboxylic acid (100 mg, 0.39 mmol), L-tert-leucinamide (Preparation 1, 78.4 mg, 0.60 mmol), EDC.HCl (115.6 mg, 0.60 mmol), HOBt (81.4 mg, 0.60 mmol) and N,N-diisopropylethylamine (0.35 mL, 2.01 mmol) in dry DMF (5 mL) was stirred at room temperature for 18 h. Then after completion of the reaction, water was added to the reaction mixture and extracted with ethyl acetate. The organic layer was separated, dried over sodium sulfate and concentrated under reduced pressure to give crude material, which was purified by column chromatography over silica gel 70% ethyl acetate-hexane as eluant to afford N-[(1S)-1-(aminocarbonyl)-2,2-dimethylpropyl]-1-(pyridin-2-ylmethyl)-1H-indazole-3-carboxamide as white solid (105 mg, yield 73%).

¹H NMR (400 MHz, DMSO-d₆) δ: 0.97 (s, 9H), 4.45 (d, J=9.6 Hz, 1H), 5.89 (br s, 2H), 7.16 (d, J=7.6 Hz, 1H), 7.27-7.31 (m, 3H), 7.43-7.45 (m, 1H), 7.57 (d, J=9.6 Hz, 1H), 7.71-7.76 (m, 3H), 8.18 (d, J=8.0 Hz, 1H), 8.48 (d, J=4.8 Hz, 1H). FIA-MS: 366.4 [M+H]⁺, 388.3 [M+H+Na]⁺.

### Example 7

### N-[(1S)-1-(aminocarbonyl)-2,2-dimethylpropyl]-1-benzyl-5-bromo-1H-indazole-3-carboxamide

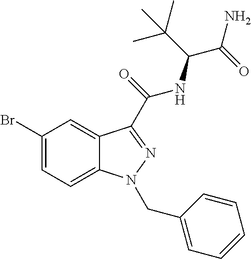

### Step 1: Methyl 1-benzyl-5-bromo-1H-indazole-3-carboxylate

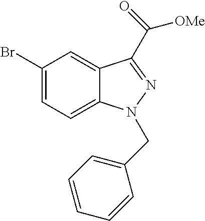

To a slurry of 60% sodium hydride (0.157 g, 3.92 mmol) in dry THF (15 mL) was added methyl 6-bromo-1H-indazole-3-carboxylate (1.0 g, 3.92 mmol). During addition gas is evolved. After stirring under nitrogen at room temperature for 30 minutes benzyl bromide (0.68 g, 3.98 mmol) was added and the mixture stirred at room temperature overnight. The mixture was partitioned between brine and ethyl acetate. The layers were separated and the organic phase washed with brine, dried over anhydrous magnesium sulfate and concentrated under reduced pressure. The residue was purified via flash chromatography on silica gel (70 g) using 30% ethyl acetate in hexanes as eluent to give 0.996 g (73.6%) of the title compound: ¹H NMR (400 MHz, CDCl₃) δ ppm 4.08 (s, 3H) 5.68 (s, 2H) 7.24 (dd, J=7.51, 1.71 Hz, 2H) 7.31-7.38 (m, 3H) 7.43 (dd, J=8.53, 1.37 Hz, 1H) 7.54-7.58 (m, 1H) 8.13 (d, J=8.53 Hz, 1H).

### Step 2: 1-Benzyl-5-bromo-1H-indazole-3-carboxylic acid

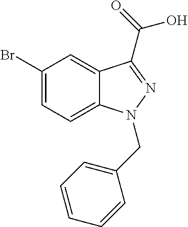

To a mixture of methyl 1-benzyl-5-bromo-1H-indazole-3-carboxylate (0.907 g, 2.63 mmol) in methanol (30 mL) was added 1N NaOH (5.0 mL, 5.0 mmol). The mixture was heated to 50° C. for 2.5 h then cooled to room temperature. The mixture was acidified to pH 4 with 1N HCl and extracted twice with ethyl acetate (30 mL). The ethyl acetate extracts were combined, dried over anhydrous magnesium sulfate and concentrated under reduced pressure and dried to give 0.7969 g (91.6%) of the title compound: ¹H NMR (400 MHz, DMSO-d₆) δ ppm 5.59 (d, J=3.07 Hz, 2H) 7.10-7.17 (m, 3H) 7.18-7.26 (m, 4H) 7.31-7.37 (m, 1H) 8.33-8.40 (m, 1H).

### Step 3: N-[(1S)-1-(aminocarbonyl)-2,2-dimethylpropyl]-1-benzyl-5-bromo-1H-indazole-3-carboxamide

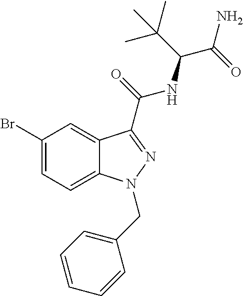

To a mixture of 1-benzyl-5-bromo-1H-indazole-3-carboxylic acid (0.7969 g, 2.406 mmol) in THF (20 mL) was added L-tert-leucinamide hydrochloride (Preparation 1, 0.401 g, 2.41 mmol), diisopropylethylamine (1.5 mL, 2.41 mmol) and HATU (0.915 g, 2.41 mmol). The mixture was stirred at room temperature for 3 h then partitioned between brine and ethyl acetate. The layers were separated and the organic phase washed with brine, dried over anhydrous magnesium sulfate and concentrated under reduced pressure. The mixture contains some tetramethyl urea from the HATU. The residue was dissolved in dichloromethane and washed 6 times with brine, dried over anhydrous magnesium sulfate and concentrated under reduced pressure. The residue was purified via flash chromatography on silica gel (70 g) using 50:40:10 ethyl acetate: dichloromethane: hexanes as eluent to give 0.7598 g (71%) of the title compound: ¹H NMR (400 MHz, CDCl₃) 5 ppm 1.17 (s, 9H) 4.57 (d, J=9.22 Hz, 1H) 5.57 (br. s., 1H) 5.63 (s, 2H) 6.02 (br. s., 1H) 7.16-7.25 (m, 3H) 7.30-7.38 (m, 3H) 7.44 (dd, J=8.88, 1.71 Hz, 1H) 7.70 (d, J=9.56 Hz, 1H) 8.54 (d, J=1.71 Hz, 1H).

### Example 8

### N-[(1S)-1-(Aminocarbonyl)-2,2-dimethylpropyl]-1-benzyl-5-pyridin-3-yl-1H-indazole-3-carboxamide

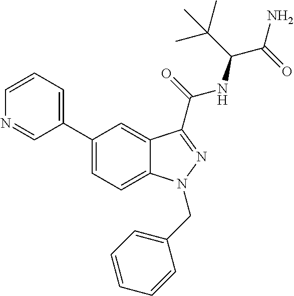

To a mixture of N-[(1S)-1-(aminocarbonyl)-2,2-dimethylpropyl]-1-benzyl-5-bromo-1H-indazole-3-carboxamide (Example 25, 0.1011 g, 0.228 mmol) in 1,4-dioxane (5.0 mL) and water (2.0 mL) was added di potassium phosphate (0.12 g, 0.684 mmol) and 3-pyridineboronic acid (0.0841 g, 0.684 mmol). Nitrogen gas was bubbled through the mixture for 5 minutes at which time 1,1′-bis(diphenylphosphino)ferrocene palladium dichloride (0.018 g, 0.025 mmol) was added and the mixture heated to 80° C. under nitrogen atmosphere overnight. The mixture was removed from heat and cooled to room temperature. The mixture was partitioned between brine and ethyl acetate, the layers were separated and the aqueous phase extracted with ethyl acetate. The combined ethyl acetate extracts were washed four times with brine, dried over anhydrous magnesium sulfate and concentrated under reduced pressure. The residue was purified via flash chromatography on silica gel (20 g) using ethyl acetate as eluent to give 0.0633 g (63%) of the title compound: MS (ESI⁺) for C26H27N5O2 m/z 442.2243 (M+H)⁺; ¹H NMR (400 MHz, DMSO-d₆) δ ppm 0.97 (s, 9H) 4.44 (d, J=10.25 Hz, 1H) 5.79 (s, 2H) 7.17 (br. s., 1H) 7.21-7.27 (m, 3H) 7.27-7.34 (m, 2H) 7.45 (dd, J=8.05, 5.12 Hz, 1H) 7.61 (d, J=9.52 Hz, 1H) 7.66 (br. s., 1H) 7.76 (dd, J=8.79, 2.20 Hz, 1H) 7.82-7.90 (m, 1H) 8.00-8.08 (m, 1H) 8.37 (s, 1H) 8.49-8.59 (m, 1H) 8.85 (d, J=1.46 Hz, 1H).

### Example 9

### N-{[1-(4-fluorobenzyl)-1H-indazol-3-yl]carbonyl}-3-methyl-L-valylglycine

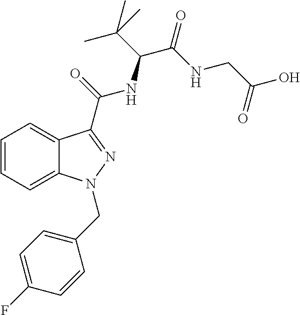

### Step 1: ((S)-2-{[1-(4-fluorobenzyl)-1H-indazole-3-carbonyl]-amino}-3,3-dimethylbutyryl-amino)acetic acid benzyl ester

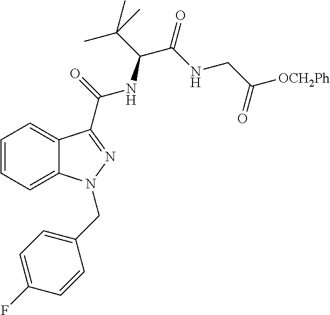

To a solution of 1-(4-fluorobenzyl)-1H-indazole-3-carboxylic acid (Example 1, Step 2, 114 mg, 0.42 mmol) in dry DMF (5 mL), N,N-diisopropylethylamine (0.5 mL, 2.96 mmol), EDC.HCl (121 mg, 0.63 mmol), HOBT (86 mg, 0.63 mmol) was added and stirred at room temperature under nitrogen atmosphere for 1 h. (2-Amino-3,3-dimethyl butyrylamino)acetic acid benzyl ester hydrochloride (Preparation 3, 200 mg, 0.63 mmol) was then added and the stirring was continued for 18 h at room temperature. On completion of reaction (monitored by TLC, Rf=0.5; solvent system 30% ethyl acetate in hexane, spots visualized with either UV or Iodine), the solution was diluted with water (50 mL), extracted with ethyl acetate (50 mL), washed with brine (25 mL). The organic layer was dried over anhydrous sodium sulfate and concentrated under reduced pressure to obtain crude product (200 mg). The crude mixture was subjected to column chromatography using 100-200 mesh silica gel, eluting with 15-20% ethyl acetate-hexane to afford ((S)-2-{[1-(4-fluorobenzyl)-1H-indazole-3-carbonyl]-amino}-3,3-dimethylbutyrylamino) acetic acid benzyl ester as sticky semi solid (193 mg, yield 83%).

¹H NMR (400 MHz, DMSO-d₆) δ: 0.99 (s, 9H), 3.87-3.93 (dd, J=8.4, 17.2 Hz, 1H), 3.99-4.05 (dd, J=6, 17.6 Hz, 1H), 4.57 (d, J=10 Hz, 1H), 5.12 (s, 2H), 5.78 (s, 2H), 7.15 (t, J=8.8 Hz, 2H), 7.28-7.35 (m, 8H), 7.46 (t, J=8 Hz, 1H), 7.61 (d, J=9.6 Hz, 1H), 7.79 (d, J=8.8 Hz, 1H), 8.17 (d, J=8 Hz, 1H), 8.80 (t, J=6 Hz, 1H). FIA-MS: 531.0 [M+H]⁺, 553.3 [M+H+Na]⁺.

### Step 2: N-{[1-(4-fluorobenzyl)-1H-indazol-3-yl]carbonyl}-3-methyl-L-valylglycine

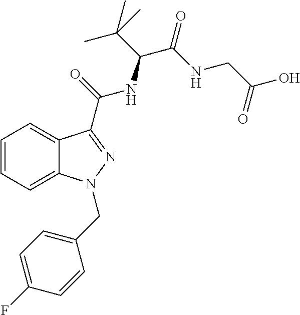

To a solution of ((S)-2-{[1-(4-fluorobenzyl)-1H-indazole-3-carbonyl]-amino}-3,3-dimethylbutyryl-amino)acetic acid benzyl ester (96 mg, 0.181 mmol) in absolute ethanol (5 mL), purged with nitrogen gas, 10% palladium on carbon (10 mg) was added and resulting mixture was stirred at room temperature under hydrogen (1 atm) for 5 h. On completion of reaction (monitored by TLC, Rf=0.1; solvent system ethyl acetate, spots visualized with either UV or Iodine), mixture was filtered through celite bed, and the filtrate evaporated to give N-{[1-(4-fluorobenzyl)-1H-indazol-3-yl]carbonyl}-3-methyl-L-valylglycine as white solid (40 mg, yield 50.6%).

¹H NMR (400 MHz, DMSO-d₆) δ: 1.00 (s, 9H), 3.75 (dd, J=6, 18 Hz, 1H), 3.85 (dd, J=6, 17 Hz, 1H), 4.56 (d, J=10 Hz, 1H), 5.78 (s, 2H), 7.16 (m, 2H), 7.27-7.33 (m, 3 H), 7.46 (t, J=8 Hz, 1H), 7.62 (d, J=10 Hz, 1H), 7.80 (d, J=9 Hz, 1H), 8.17 (d, J=8 Hz, 1H), 8.67 (t, J=6 Hz, 1H). FIA-MS: 441.2 [M+H]⁺, 463.2 [M+H+Na]⁺.

### Example 10

### 1-(4-cyanobenzyl)-7-fluoro-N-[(1S)-1-{[(2-hydroxyethyl)amino]carbonyl}-2,2-dimethylpropyl]-1H-indazole-3-carboxamide

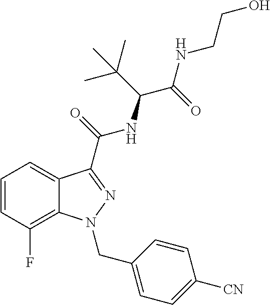

### Step 1: 7-Fluoro-1H-indazole-3-carboxylic acid

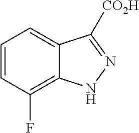

This compound was prepared following the procedure of Johnson, B. L.; Rodgers, J. D. Syn. Comm. 2005, 35, 2681-2684. A suspension of 5.28 g 7-fluoroisatin in 30 mL of water was added 1.30 g NaOH, in 10 mL water with stirring. The resulting dark red solution was stirred until all of the solids dissolved and was then cooled in an ice water bath. The solution was then slowly added a cooled (ice bath) solution of 2.21 g NaNO₂ in 10 mL water. These combined solutions were then added slowly to cooled (ice bath) to solution of aqueous sulfuric acid (3.4 mL H₂SO₄ in 60 mL water). Ice was added to maintain a temperature of approximately 0° C. After stirring for approximately 10 minutes, this dark red diazonium solution was added slowly to a chilled (0° C., ice bath) solution of 18 g SnCl₂2H₂O in 30 mL concentrated HCl. Ice was again added to maintain a temperature of approximately 0° C. After stirring for approximately 1 hour, the reaction was filtered and the resulting residue was dissolved in 1 N NaOH (60 mL), washed with ether (2×50 mL). The resulting yellow-brown solution was cooled in an ice bath and acidified to a pH˜3 (litmus paper) with concentrated HCl, which resulted in the formation of a dark yellow precipitate. The precipitate was collected by filtration, washed with water, and dried over night in an oven to give 3.69 g (47%) of 7-fluoro-1H-indazole-3-carboxylic acid as an orange solid. ¹H NMR (400 MHz, DMSO-d₆) δ 14.35 (br s, 1H), 13.22 (br s, 1H), 7.89-7.87 (m, 1H), 7.26-7.21 (m, 2H). MS (ESI) m/z 181 (M+H)⁺.

### Step 2: Methyl 7-Fluoro-1H-indazole-3-carboxylate

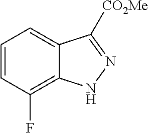

A solution of 30 g 7-fluoro-1H-indazole-3-carboxylic acid in 1200 mL dry methanol was added 8 mL concentrated sulfuric acid. The resulting mixture was heated to reflux and was continued over night. Reaction was allowed to cool to room temperature and was diluted with ethyl acetate (1000 mL). Organic solution was washed with saturated NaHCO₃ (2×250 mL), brine (2×250 mL), dried (MgSO₄), filtered and concentrated to a brown solid. Crude reaction was purified via MPLC (5%-30% ethyl ether/heptane) to afford 20.74 g (64%) of methyl 7-fluoro-1H-indazole-3-carboxylate as a bright yellow solid. ¹H NMR (400 MHz, DMSO-d₆) δ 14.49 (br s, 1H), 7.85-7.83 (m, 1H), 7.28-7.21 (m, 2H), 3.92 (s, 3H). MS (ESI) m/z 195 (M+H)⁺.

### Step 3: Methyl 1-(4-cyanobenzyl)-7-fluoro-1H-indazole-3-carboxylate

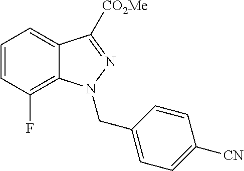

A suspension of 1.67 g of 60% sodium hydride in 134.0 mL dry DMF was added 7 g methyl 7-fluoro-1H-indazole-3-carboxylate in 10 mL dry DMF drop wise via syringe at room temperature. The mixture was allowed to stir for approximately 1 h at room temperature and was then added 8.02 g of 4-cyanobenzyl bromide in 56 mL DMF drop wise via syringe. The resulting mixture was then heated to 60° C. and allowed to stir over night. Reaction was allowed to cool to room temperature and was quenched by the careful addition of water (500 mL). The aqueous solution was extracted with ethyl acetate (4×150 mL). The organic solution is washed with brine (2×200 mL), dried (MgSO₄), filtered and concentrated to an oil. Crude reaction was purified via MPLC (25%-50% ethyl ether/heptane) to afford 7.68 g (68.8%) of methyl 1-(4-cyanobenzyl)-7-fluoro-1H-indazole-3-carboxylate as a light yellow solid. ¹H NMR (400 MHz, CDCl₃) δ 8.01 (d, J=8.0 Hz, 1H), 7.60 (d, J=7.8 Hz, 2H), 7.36 (d, J=8.0 Hz, 2H, 7.20-7.28 (m, 1H), 7.06-7.14 (m, 1H), 5.85 (s, 2H), 4.06 (s, 3H). MS (ESI) m/z 310 (M+H)⁺.

### Step 4: 1-(4-cyanobenzyl)-7-fluoro-1H-indazole-3-carboxylic acid

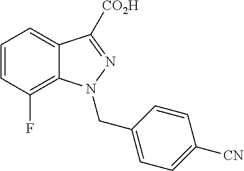

A solution of 6.07 g of methyl 1-(4-cyanobenzyl)-7-fluoro-1H-indazole-3-carboxylate in 100 mL THF was added 20 mL of 2.5 M sodium hydroxide at room temperature. The resulting mixture was allowed to stir overnight. Reaction was diluted with 150 mL water and the aqueous solution was washed with ethyl ether (3×50 mL). The aqueous solution was cooled in an ice bath and acidified with concentrated HCl to a pH-3 to afford a white precipitate. The precipitate was collected by filtration, washed with water and dried under reduced pressure to afford 5.42 g (94%) of 1-(4-cyanobenzyl)-7-fluoro-1H-indazole-3-carboxylic acid as a white solid. ¹H NMR (400 MHz, DMSO-d₆) δ 13.38 (br s, 1H), 7.93-7.92 (m, 1H), 7.79 (d, J=8.2 Hz, 2H), 7.33-7.26 (m, 4H), 5.90 (s, 2H). MS (ESI) m/z 195 (M+H)⁺.

### Step 4: 1-(4-cyanobenzyl)-7-fluoro-N-[(1S)-1-{[(2-hydroxyethyl)amino]carbonyl}-2,2-dimethylpropyl]-1H-indazole-3-carboxamide

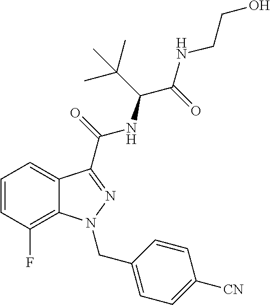

A solution of 1.05 g 1-(4-cyanobenzyl)-7-fluoro-1H-indazole-3-carboxylic acid and 3.1 mL of N,N-diisopropylethylamine in 18 mL of DMF was added 1.66 g HATU with stirring. The resulting mixture was allowed to stir for 10 min, and was then added 908 mg of (S)-2-Amino-N-(2-hydroxyethyl)-3,3-dimethylbutyramide hydrochloride (Preparation 4). The resulting tan solution was allowed to stir at room temperature over night. The dark brown reaction mixture was, diluted with water (100 mL). The aqueous solution was extracted with ethyl acetate (3×25 mL). The combined organic solutions were washed with brine (2×25 mL), dried (MgSO₄), filtered and concentrated under reduced pressure to give a dark brown oil. Crude reaction was purified via MPLC (25-50% ethyl acetate/heptane) to afford 1.27 g (80%) of 1-(4-cyanobenzyl)-7-fluoro-N-[(1S)-1-{[(2-hydroxyethyl)amino]carbonyl}-2,2-dimethylpropyl]-1H-indazole-3-carboxamide as an off white solid. ¹H NMR (400 MHz, DMSO-d₆) δ ppm 8.32 (t, J=5.5 Hz, 1H), 7.99-8.06 (m, 1H), 7.81 (d, J=8.2 Hz, 1H), 7.66 (d, J=9.7 Hz, 1H), 7.22-7.37 (m, 3H), 5.94 (s, 2H), 4.69 (t, J=5.1 Hz, 1H), 4.51 (d, J=9.7 Hz, 1H), 3.41 (q, J=5.7 Hz, 2H), 3.07-3.27 (m, 2H), 0.97 (s, 9H). MS (ESI) m/z 195 (M+H)⁺. MS (ESI) m/z 452 (M+H)⁺.

### Example 11

### N-{(1S)-1-[({[5-(aminocarbonyl)-1,3,4-oxadiazol-2-yl]methyl}amino)carbonyl]-2,2-dimethylpropyl}-1-(4-fluorobenzyl)-1H-indazole-3-carboxamide

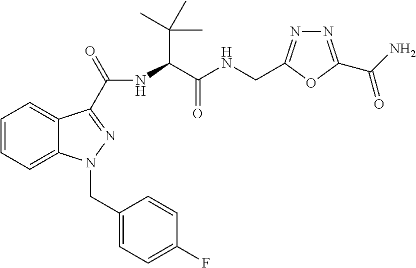

To a solution of 1-(4-fluorobenzyl)-1H-indazole-3-carboxylic acid (Example 1, Step 2, 200 mg, 0.74 mmol) in dichloromethane (2 mL) was added TBTU (356 mg, 1.11 mmol) and triethylamine (0.52 mL, 3.70 mmol). After fifteen minutes of stirring at ambient temperature, (S)-5-((2-amino-3,3-dimethylbutanamido)methyl)-1,3,4-oxadiazole-2-carboxamide trifluoroacetate (Preparation 27, 328 mg, 0.89 mmol) was added and stirring continued for one hour. The reaction was quenched with water and the biphasic solution was filtered through a phase separator tube. The resulting organic solution was concentrated to provide the crude product as an oil. The crude material was purified using chromatography over silica gel (heptane/ethyl acetate) to provide N-{(1S)-1-[({[5-(aminocarbonyl)-1,3,4-oxadiazol-2-yl]methyl}amino)carbonyl]-2,2-dimethylpropyl}-1-(4-fluoro-benzyl)-1H-indazole-3-carboxamide as a colorless oil (95 mg, 25% yield).

¹H NMR (400 MHz, DMSO-d₆) δ ppm 0.98 (s, 9H) 4.47-4.73 (m, 3H) 5.77 (s, 2H) 7.15 (t, J=8.79 Hz, 2H) 7.23-7.38 (m, 3H) 7.45 (t, J=7.69 Hz, 1H) 7.62 (d, J=10.25 Hz, 1H) 7.79 (d, J=8.79 Hz, 1H) 8.16 (d, J=8.79 Hz, 1H) 8.19 (br. s., 1H) 8.59 (s, 1H) 9.16 (t, J=5.49 Hz, 1H); LC-MS: 508 [M+H]⁺

### Example 12

### N-[(1S)-1-(Aminocarbonyl)-2,2-dimethylpropyl]-1-(4-fluorobenzyl)-1H-indazole-3-carboxamide

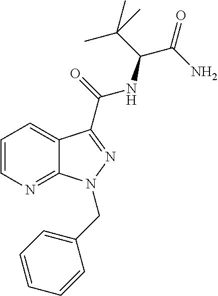

### Step 1: Ethyl 1H-pyrazolo[3,4-b]pyridine-3-carboxylate

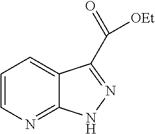

1H-Pyrazolo[3,4-b]pyridine-3-carboxylic acid (prepared according to the procedure in the literature; Lynch, B. M. et al, Can. J. Chem. 1988, 66, 420-428; 2 g, 9 mmol) was suspended in ethanol (60 mL) and purged with HCl gas for 5 min. The resultant mixture was stirred at room temperature overnight. The reaction mixture was concentrated, diluted with water, neutralized with 2M Na₂CO₃ solution, and extracted with ethyl acetate(3×20 mL). The combined organic layers were concentrated and the residue was purified by chromatography using 40-60% ethyl acetate/hexane as eluent to give ethyl 1H-pyrazolo[3,4-b]pyridine-3-carboxylate as light brown solid (904 mg, 40%). LC-MS; 228, [M+H]⁺.

### Step 2: Ethyl 1-benzyl-1H-pyrazolo[3,4-b]pyridine-3-carboxylate

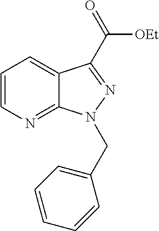

A solution of ethyl 1H-pyrazolo[3,4-b]pyridine-3-carboxylate (1.19 g, 5.23 mmol) in DMF(10 mL) was added dropwise to a suspension of NaH (230 mg, 5.75 mmol) in DMF (10 mL). The reaction mixture was heated to 50° C. for 45 min, then a solution of benzyl bromide (1.79 g, 10.5 mmol) in 10 mL of DMF was added dropwise. The reaction mixture was stirred at 50° C. overnight. The reaction was quenched by addition of water while cooling in an ice-bath, and then extracted with ethyl acetate. The organic layer was washed with brine, dried over Na₂SO₄, and concentrated. The residue was purified by chromatography over silica gel using 40-60% ethyl acetate-hexane as eluent to afford the ethyl 1-benzyl-1H-pyrazolo[3,4-b]pyridine-3-carboxylate as white solid (620 mg, 42.2%).

¹H NMR (400 MHz, DMSO-d₆) δ ppm 1.34 (t, J=7.12 Hz, 3H) 4.37 (q, J=7.25 Hz, 2H) 5.78 (s, 2H) 7.21-7.33 (m, 5H) 7.45 (dd, J=8.06, 4.57 Hz, 1H) 8.47 (dd, J=8.06, 1.61 Hz, 1H) 8.68 (dd, J=4.56, 1.61 Hz, 1H). LC-MS; 282 [M+H]⁺, 304 [M+Na]⁺.

### Step 3: 1-Benzyl-1H-pyrazolo[3,4-b]pyridine-3-carboxylic acid

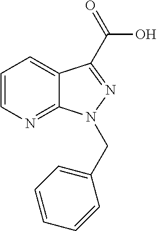

A mixture of ethyl 1-benzyl-1H-pyrazolo[3,4-b]pyridine-3-carboxylate (620 mg, 2.2 mmol), 1N NaOH (5 mL), THF (5 mL), and ethanol (5 mL) was stirred for 4 h at room temperature. The reaction was concentrated, diluted with water, and neutralized with 1N HCl solution. The resultant precipitate was collected by filtration, and air dried to give 1-benzyl-1H-pyrazolo[3,4-b]pyridine-3-carboxylic acid as white solid (525 mg, 94%).

LC-MS; 254 [M+H]⁺, 276 [M+Na]⁺.

### Step 4: N-[(1S)-1-(aminocarbonyl)-2,2-dimethylpropyl]-1-benzyl-1H-pyrazolo[3,4-b]pyridine-3-carboxamide

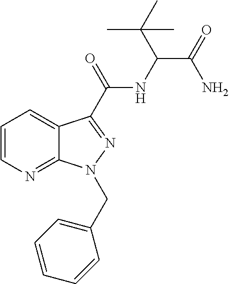

A mixture of 1-benzyl-1H-pyrazolo[3,4-b]pyridine-3-carboxylic acid (50 mg, 0.20 mmol), L-tert-leucinamide (Preparation 1, 49.4 mg, 0.30 mmol), EDC.HCl (57 mg, 0.30 mmol), HOBt (40 mg, 0.30 mmol) and N,N-diisopropylethylamine (0.17 mL, 0.98 mmol) in dry DMF (2 mL) was stirred at 50° C. overnight. The crude reaction mixture was subjected to purification by reverse-phase HPLC to afford N-[(1S)-1-(aminocarbonyl)-2,2-dimethylpropyl]-1-benzyl-1H-pyrazolo[3,4-b]pyridine-3-carboxamide as gummy solid (7.4 mg, 10%).

¹H NMR (400 MHz, DMSO-d₆) δ ppm 0.99 (s, 8H) 4.46 (d, J=9.52 Hz, 1H) 5.80 (d, J=2.93 Hz, 2H) 7.22 (br. s., 1H) 7.25-7.34 (m, 3H) 7.41 (dd, J=8.05, 4.39 Hz, 1H) 7.63 (d, J=9.52 Hz, 1H) 7.68 (br. s., 1H) 8.56 (d, J=9.15 Hz, 1H) 8.68 (d, J=4.39 Hz, 1H); LC-MS: 365 [M+H]⁺.

Preparations:

### Preparation 1: L-tert-leucinamide

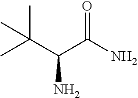

### Step 1: Benzyl [(1S)-1-(aminocarbonyl)-2,2-dimethylpropyl]carbamate

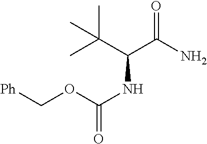

To a solution of N-[(benzyloxy)carbonyl]-tert-leucine (prepared according to the procedure in the literature; Emily, M. S. et al. Tetrahedron 2001, 57, 5303-5320; 3.7 g, 14 mmol) in DMF (80 mL) were added ammonium chloride (900 mg, 17 mmol), triethylamine (5.9 mL, 42 mmol), HOBt (2.8 g, 18 mmol), and EDC (3.1 g, 18 mmol) at rt. After 17 h, the reaction mixture was quenched by addition of sat. aq. sodium bicarbonate (100 mL) and extracted with ethyl acetate (100 mL×3). The combined organic layers were washed with water (100 mL×3), brine (50 mL), dried over sodium sulfate, filtered and concentrated in vacuo. The residue was purified by column chromatography on silica gel eluting with hexane/ethyl acetate (2/1-1/1) to afford 3.0 g (82%) of the title compound. MS (ESI) m/z 265 (M+H)⁺.

### Step 2: L-tert-Leucinamide

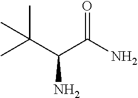

To a solution of benzyl [(1S)-1-(aminocarbonyl)-2,2-dimethylpropyl]carbamate (3.7 g, 14 mmol) in THF (40 mL) was added 10% Pd/C (710 mg). The flask was evacuated and flushed with H₂ gas and this process was repeated three times. The flask was filled with H₂ gas (4 atm) and stirred for 3 h at rt. Then the reaction mixture was filtered through a pad of Celite and concentrated in vacuo to give the title compound as white solid (crude; 1.8 g). ¹H-NMR (300 MHz, DMSO-d₆) [symbol] 6.59 (bs, 1H), 5.92 (bs, 1H), 3.12 (s, 1H), 1.02 (s, 1H). MS (ESI) m/z 131 (M+H)⁺.

### Preparation 2: (S)-2-Amino-N-carbamoylmethyl-3,3-dimethylbutyramide hydrochloride

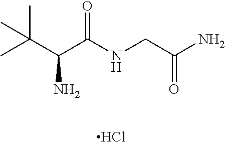

### Step 1: [(S)-1-(Carbamoylmethylcarbamoyl)-2,2-dimethylpropyl]carbamic acid tent-butyl ester

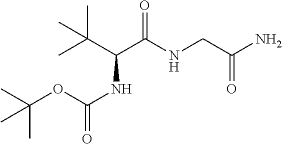

To a solution of N-Boc-L-tert-leucine (1.0 g, 4.327 mmol) in dry DMF (10 ml), N,N-diisopropylethyl amine (5.1 ml, 30.3 mmol), EDC.HCl (1.23 g, 6.5 mmol), HOBT (880 mg, 6.5 mmol) was added and stirred at it under nitrogen atmosphere for 30 min. Glycinamide hydrochloride (720 mg, 6.5 mmol) was then added to it and stirring was continued for 18 h at rt. On completion of reaction (monitored by TLC, R_{f}=0.3; solvent system 40% ethyl acetate in hexane, spots visualized with either KMnO₄ or Iodine), the solution was diluted with distilled water (100 ml), extracted with ethyl acetate (100 ml), washed with brine (50 ml), dried over anhydrous Na₂SO₄ and concentrated under reduced pressure to obtain crude product (1.6 g). The crude mixture was subjected to column chromatography using 100-200 mesh silica gel, eluting with 30-50% ethyl acetate-hexane to afford desired product [(S)-1-(Carbamoylmethylcarbamoyl)-2,2-dimethylpropyl]-carbamic acid tert-butyl ester as gummy sticky mass (1.09 g, yield 87.9%).

### Step 2: (S)-2-Amino-N-carbamoylmethyl-3,3-dimethylbutyramide hydrochloride

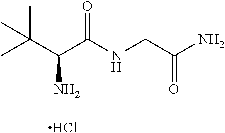

[(S)-1-(Carbamoylmethylcarbamoyl)-2,2-dimethyl-propyl]carbamic acid tert butyl ester (1.09 g, 3.79 mmol) was dissolved in 40 ml of 4N 1,4-dioxane-HCl solution and stirred at rt under nitrogen atmosphere for 4 hr. On completion of reaction (monitored by TLC, R_{f}=0.1; solvent system 50% ethyl acetate in hexane, spots visualized with UV), dioxane was removed under reduced pressure to afford desired product (S)-2-Amino-N-carbamoylmethyl-3,3-dimethylbutyramide hydrochloride as gummy semi solid (750 mg, yield 88%). ¹H NMR (400 MHz, DMSO-d₆) δ: 0.99 (s, 9H), 3.56-3.59 (m, 1H), 3.69-3.72 (m, 2H), 7.10 (br s, 1H), 7.47 (br s, 1H), 8.25 (br s, 3H), 8.73 (br s, 1H). FIA-MS: 188.2 [M+H]⁺.

### Preparation 3: ((S-2-Amino-3,3-dimethyl-butyrylamino)acetic acid benzyl ester hydrochloride

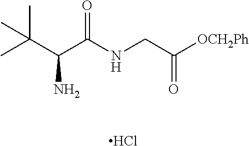

### Step 1: ((S)-2-tert-Butoxycarbonylamino-3,3-dimethylbutyrylamino)acetic acid benzyl ester

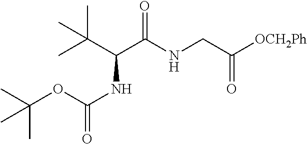

To a solution of N-Boc-L-tert-leucine (1.5 g, 6.48 mmol) in dry DMF (40 mL) N,N-diisopropylethylamine (8.0 mL, 45.34 mmol), EDC.HCl (1.89 g, 9.89 mmol) and HOBt (1.34 g, 9.89 mmol) were added under nitrogen atmosphere, and stirred at room temperature for 1 h. Then glycine benzyl ester (as p-toluenesulfonic acid salt) (3.33 g, 9.89 mmol) was added to the reaction mixture and stirred at room temperature for additional 18 h. After completion of the reaction (monitored by TLC, 30% ethyl acetate in hexane, R_{f} for product 0.5, spots visualized with UV and iodine), water (400 ml) was added to the reaction mixture and extracted with ethyl acetate (400 ml). The organic layer was separated, dried over sodium sulfate and concentrated under reduced pressure to give crude material (2.7 g), which on column chromatography over silica gel (100-200 mesh) using 20% ethyl acetate-hexane as eluant afforded ((S)-2-tert-butoxycarbonylamino-3,3-dimethylbutyrylamino)acetic acid benzyl ester as white solid (2.0 g, yield 82%).

¹H NMR (400 MHz, CDCl₃) δ: 0.99 (s, 9H), 1.41 (s, 9H), 3.87 (d, J=8.8 Hz, 1H), 3.93-3.97 (m, 1H), 4.17-4.21 (m, 1H), 5.14-5.23 (m, 3H), 6.19 (s, 1H), 7.31-7.38 (m, 5H). FIA-MS: 379.0 [M+H]⁺, 396.1 [M+H+NH₃]⁺, 401.2 [M+H+NH₃]⁺.

### Step 2: ((S)-2-Amino-3,3-dimethylbutyrylamino)acetic acid benzyl ester hydrochloride

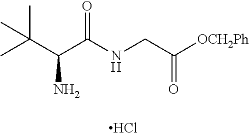

((S)-2-tert-Butoxycarbonylamino-3,3-dimethylbutyrylamino)acetic acid benzyl ester (2.0 g, 5.29 mmol) was dissolved in 16 mL of 4NHCl-1,4-dioxane solution and stirred at room temperature under nitrogen atmosphere for 4 h. Upon completion of reaction (monitored by TLC, R_{f}=0.1; solvent system 30% ethyl acetate in hexane, spots visualized with UV), dioxane was removed under reduced pressure to afford ((S)-2-amino-3,3-dimethylbutyrylamino)acetic acid benzyl ester hydrochloride as off-white solid (1.6 g, yield 96%).

¹H NMR (400 MHz, CDCl₃) δ: 1.09 (s, 9H), 3.69 (m, 3H), 5.10 (s, 2H), 7.30-7.36 (m, 5H), 8.01 (brs, 3H), 8.60 (br s, 1H).

The following intermediates were prepared in a similar manner:

| Compound Name | Structure | Analytical Data |
|---|---|---|
| Preparation 4: (S)-2-Amino-N-(2-hydroxyethyl)- 3,3-dimethylbutyramide hydrochloride | 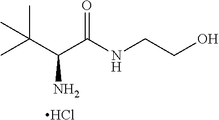 | ¹H NMR (400 MHz, DMSO-d₆) δ ppm: 0.97 (s, 9H), 3.08-3.22 (m, 1H), 3.25-3.33 (m, 1H), 3.38-3.56 (m, 3H), 4.79 (br s, 1H), 8.14 (br s, 3H), 8.52 (t, J = 5.6 Hz, 1H). FIA-MS: 175.2 [M + H]⁺. |
| Preparation 5: (S)-2-Amino-N-(3-hydroxypropyl)- 3,3-dimethylbutyramide hydrochloride | 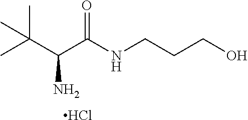 | ¹H NMR (400 MHz, DMSO-d₆) δ ppm: 0.97 (s, 9H), 1.56-1.60 (m, 2H), 3.09-3.16 (m, 1H), 3.24-3.28 (m, 1H), 8.10 (br s, 3H), 8.45 (br s, 1H). FIA-MS: 189.4 [M + H]⁺. |
| Preparation 6: (S)-2-Amino-N-cyclopropyl-3,3- dimethylbutyramide hydrochloride | 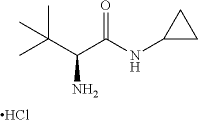 | ¹H NMR (400 MHz, CDCl₃) δ ppm: 0.63-0.73 (m, 4H), 1.15 (s, 9H), 2.44 (br s, 1H), 2.75 (br s, 1H), 8.13 (br s, 3H), 8.30 (br s, 1H). FIA-MS: 171.2 [M + H]⁺. |
| Preparation 7: (S)-2-Amino-N-cyclobutyl-3,3- dimethylbutyramide hydrochloride | 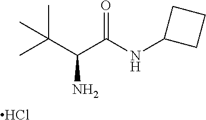 | ¹H NMR (400 MHz, CDCl₃) δ ppm: 1.15 (s, 9H), 1.74 (m, 2H), 1.93-2.29 (m, 5H), 4.31 (m, 1H), 8.10 (br s, 4H). FIA-MS: 185.3 [M + H]⁺. |
| Preparation 8: N-[(2S)-2,3-dihydroxypropyl]-3- methyl-L-valinamide hydrochloride | 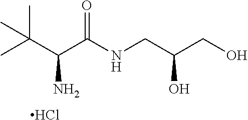 | 1H NMR (400 MHz, DMSO-d₆) δ ppm: 0.99 (s, 9H) 2.87-2.96 (m, 1H) 3.33 (ddd, J = 19.74, 5.47, 5.28 Hz, 2H) 3.43 (td, J = 6.64, 4.30 Hz, 1H) 3.49-3.57 (m, 2H) 8.12 (br. s., 3H) 8.44 (t, J = 5.67 Hz, 1H). LC/MS 205.1 (M + H). |
| Preparation 9: N-[(2R)-2,3-dihydroxypropyl]-3- methyl-L-valinamide hydrochloride | 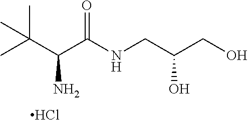 | ¹H NMR (400 MHz, DMSO-d₆) δ ppm 0.84 (s, 9H) 1.58 (br. s., 2H) 2.83 (s, 1H) 3.00 (dd, J = 12.76, 6.85 Hz, 1H) 3.10-3.17 (m, 1H) 3.13 (d, J = 5.91 Hz, 1H) 3.26 (d, J = 3.76 Hz, 2H) 3.30 (s, 1H) 3.39-3.48 (m, J = 10.44, 5.50, 5.37, 5.27 Hz, 1H) 4.50 (t, J = 5.24 Hz, 1H) 4.70 (d, J = 4.56 Hz, 1H) 7.70 (t, J = 5.77 Hz, 1H). FIA-MS: 205.1 [M + H]⁺. |
| Preparation 10: (S)-2-Amino-N-(1,3-dihydroxy-2- propyl)-3,3-dimethylbutyramide hydrochloride | 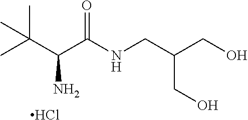 | 1H NMR (400 MHz, DMSO-d6): δ ppm 0.98 (s, 9H), 3.16 (s, 1H), 3.38-3.46 (m, 3H), 3.46-3.48 (m, 2H), 3.50-3.56 (m, 2H), 3.76-3.78 (m, 1H), 8.08 (br s, 2H), 8.22 (d, J = 8.0 Hz, 1H). LC-MS 205.4 [M + H]+. |
| Preparation 11: N-{2- [(aminocarbonyl)amino]ethyl}- 3-methyl-L-valinamide hydrochloride | 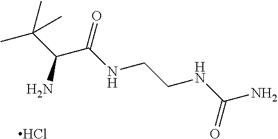 | 1H NMR (400 MHz, DMSO-d₆) δ ppm 1.00 (s, 9H) 2.37-2.44 (m, 1H) 3.01-3.17 (m, 2H) 3.19-3.30 (m, 1H) 3.49 (br. s., 1H) 8.18 (br. s., 3H) 8.57 (t, 1H) 8.72 (br. s., 2H) |
| Preparation 12: N-{2- [(cyclopropylsulfonyl)amino]ethyl}- 3-methyl-L-valinamide hydrochloride | 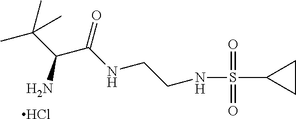 | ¹H NMR (400 MHz, DMSO-d₆) δ ppm 0.86-0.94 (m, 2H) 0.96 (s, 9H) 2.47-2.50 (m, 1H) 2.50-2.57 (m, 2H) 3.01-3.09 (m, 2H) 3.15-3.25 (m, 1H) 3.25-3.36 (m, 1H) 3.48 (d, J = 3.13 Hz, 1H) 7.14 (br. s., 1H) 8.14 (d, J = 2.15 Hz, 2H) 8.60 (t, J = 5.57 Hz, 1H); LC-MS: 392 [M + H]⁺, |
| Preparation 13: N-{2- [(cyclopropylcarbonyl)amino]ethyl}- 3-methyl-L-valinamide hydrochloride | 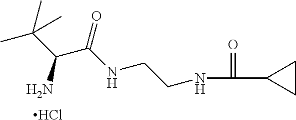 | ¹H NMR (400 MHz, DMSO-d₆) δ ppm 0.56-0.70 (m, 4H) 0.98 (s, 9H) 1.56-1.68 (m, 1H) 3.06-3.31 (m, 4H) 3.50 (dd, 1H) 8.31 (d, J = 2.35 Hz, 2H) 8.43 (t, 1H) 8.73 (t, J = 4.59 Hz, 1H) LC-MS: 278 [M + H]⁺, 300 [M + Na]⁺. |
| Preparation 14: 3-methyl-N-[2- (methylsulfonyl)ethyl]-L-valinamide hydrochloride | 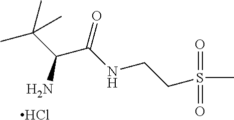 | ¹H NMR (400 MHz, DMSO-d₆) δ ppm 0.98 (s, 9H) 3.23-3.36 (m, 2H) 3.44-3.53 (m, 2H) 3.56 (s, 3H) 8.23 (d, J = 3.91 Hz, 3H) 8.90 (t, J = 5.67 Hz, 1H). FIA-MS: 237.1 [M + H]⁺. |
| Preparation 15: N-[2-(Aminosulfonyl)ethyl]-3- methyl-L-valinamide hydrochloride | 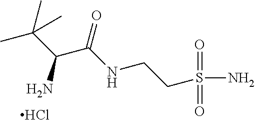 | ¹H NMR (400 MHz, DMSO-d₆) δ ppm 0.98 (s, 9H) 3.10-3.22 (m, 2H) 3.41-3.53 (m, 2H) 3.57 (s, 3H) 6.97 (s, 1H) 8.17 (d, J = 2.74 Hz, 2H) 8.76 (t, J = 5.67 Hz, 1H). FIA-MS: 238.1 [M + H]⁺. |
| Preparation 16: N-[(1-Hydroxycyclopropyl)methyl]- 3-methyl-L-valinamide hydrochloride | 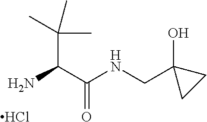 | 1H NMR (400 MHz, DMSO-d₆) δ ppm 0.94 (t, J = 7.33 Hz, 2H) 1.01 (bs, 2H) 1.04 (s, 9H) 2.34-2.56 (m, 2H) 2.69 (s, 1H) 3.68 (d, J = 5.47 Hz, 1H) 3.86-3.99 (m, 1H) 4.06-4.19 (m, 1H) 8.31 (d, J = 3.52 Hz, 2H) 8.92 (t, J = 5.47 Hz, 1H) MS: 201.2 [M + H]+ |
| Preparation 17: N-(3-Hydroxyphenyl)-3-methyl- L-valinamide hydrochloride | 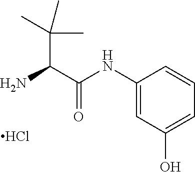 | 1H NMR (400 MHz, DMSO-d₆) δ ppm 0.93-1.08 (m, 9H) 3.91 (d, J = 5.28 Hz, 1H) 6.27-6.66 (m, 1H) 6.95-7.23 (m, 2H) 8.3 (d, J = 3.71 Hz, 1H) 8.3 (bs, 2H) 10.74 (s, 1H) MS: 222.3 [M + H]+ |
| Preparation 18: N-[(1-Hydroxycyclopentyl)methyl]- 3-methyl-L-valinamide hydrochloride | 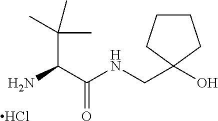 | 1H NMR (400 MHz, DMSO-d₆) δ ppm 0.85-1.08 (m, 9H) 1.39-2.05 (m, 8H) 3.24 (d, J = 11.14 Hz, 1H) 3.47-3.63 (m, 1H) 3.73 (d, J = 10.94 Hz, 1H) 8.10 (s, 1H) 8.23 (br. s., 3H); MS: 229.3 [M + H]+ |
| Preparation 19: N-[1-(Hydroxymethyl)cyclopropyl]- 3-methyl-L-valinamide hydrochloride | 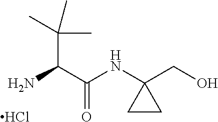 | 1H NMR (400 MHz, DMSO-d₆) δ ppm 0.34-0.79 (m, 4H) 0.96 (s, 9H) 2.44-2.57 (m, 1H) 3.28 (d, J = 11.14 Hz, 1H) 3.42 (d, J = 4.30 Hz, 1H) 3.51-3.68 (m, 1H) 8.27 (br. s., 3H) 8.75 (s, 1H) MS: 201.4 [M + H]+ |
| Preparation 20: 1-(3-Methyl-L-valyl)piperidine-3- carboxamide hydrochloride | 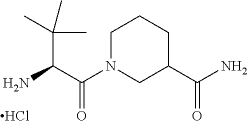 | 1H NMR (400 MHz, DMSO-d₆) δ ppm 0.98-1.09 (m, 15H) 2.51 (t, J = 5.57 Hz, 2H) 2.90 (s, 1H) 3.1 (bs, 2H) 7.44-7.63 (m, 3H) 8.39 (br. s., 1H); MS: 242.3 [M + H]+ |

### Preparation 21: (S)-5-((2-Amino-3,3-dimethylbutanamido)methyl)-1,3,4-oxadiazole-2-carboxylic acid ethyl ester, trifluoroacetate

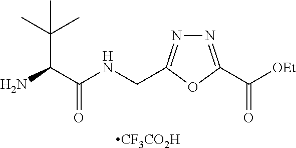

### Step 1: (S)-5-((2-(tert-butoxycarbonylamino)-3,3-dimethylbutanamido)methyl)-1,3,4-oxadiazole-2-carboxylic acid ethyl ester

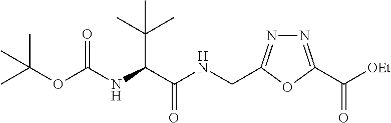

To a solution of N-Boc-L-tert-leucine (4.91 g, 21.2 mmol) in dichloromethane (50 mL) was added TBTU (10.2 g, 31.9 mmol) and triethylamine (8.88 mL, 63.7 mmol). After fifteen minutes of stirring at ambient temperature, ethyl 5-(aminomethyl)-1,3,4-oxadiazole-2-carboxylate (prepared according to the procedure in the literature; Kolb, H. C. et al. U.S. Pat. No. 6,951,946; 4.0 g, 23.0 mmol) was added and stirring continued for 18 hours. The solution was partitioned between ethyl acetate and water. The organic layer was washed with water (100 mL) and saturated sodium chloride (100 mL) and dried over magnesium sulfate. Filtration and concentration provided the crude product as a brown oil. The material was purified using normal phase chromatography (heptane/ethyl acetate) to provide the title compound as a colorless oil (5.72 g, 64% yield).

¹H NMR (400 MHz, DMSO-d₆) δ ppm 0.91 (s, 9H) 1.32 (t, 3H) 1.38 (s, 9H) 3.89 (d, J=9.38 Hz, 2H) 4.41 (q, J=7.04 Hz, 2H) 4.50-4.70 (m, 1H) 6.54 (d, J=8.99 Hz, 1H) 8.77 (t, J=5.08 Hz, 1H)

### Step 2: Ethyl (S)-5-((2-amino-3,3-dimethylbutanamido)methyl)-1,3,4-oxadiazole-2-carboxylate, trifluoroacetate salt

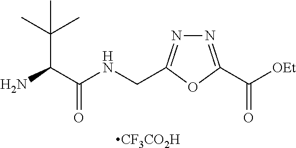

To a solution of ethyl (S)-3-((2-(tert-butoxycarbonylamino)-3,3-dimethylbutanamido)methyl)-1,2,4-oxadiazole-5-carboxylate (900 mg, 2.34 mmol) in dichloromethane (3 mL) was added trifluoroacetic acid (3 mL). The solution was stirred for one and concentrated in vacuo to provide the title compound as a brown oil (900 mg, quantitative yield). ¹H NMR (400 MHz, DMSO-d₆) δ ppm 1.00 (s, 9H) 1.34 (t, J=7.23 Hz, 3H) 3.53 (d, J=5.47 Hz, 2H) 4.43 (q, J=7.03 Hz, 2H) 4.48-4.77 (m, 1H) 8.09 (br. s., 2H) 9.07-9.22 (m, 1H). MS: 285 (M+H)

The following intermediates were prepared in a similar manner:

| Compound Name | Structure | Analytical Data |
|---|---|---|
| Preparation 22: (3R)-1-(3-Methyl- L-valyl)pyrrolidin-3- ol trifluoroacetate | 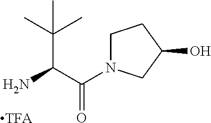 | 1H NMR (400 MHz, DMSO-d₆) δ ppm 0.99 (s, 9H) 1.69-1.95 (m, 2H) 3.22-3.30 (m, 1H) 3.37-3.58 (m, 3H) 3.66-3.76 (m, 1H) 3.88 (dd, J = 27.16, 5.28 Hz, 1H) 4.32 (d, J = 20.71 Hz, 1H) 8.02 (br. s., 2H). MS: 201 (M + H) |
| Preparation 23: Ethyl 3-{[(3-methyl- L- valyl)amino]methyl}- 1,2,4-oxadiazole- 5-carboxylate trifluoroacetate | 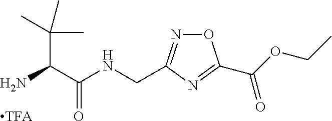 | 1H NMR (400 MHz, DMSO-d₆) δ ppm 1.00 (s, 9H) 1.34 (t, J = 7.23 Hz, 3H) 3.53 (d, J = 5.47 Hz, 2H) 4.43 (q, J = 7.03 Hz, 2H) 4.48-4.77 (m, 1H) 8.09 (br. s., 2H) 9.07-9.22 (m, 1H). MS: 285 (M + H) |
| Preparation 24: 3-Methyl-N-[(5- methyl-1,3,4- oxadiazol-2- yl)methyl]-L- valinamide trifluoroacetate | 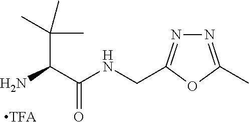 | 1H NMR (400 MHz, DMSO-d₆) δ ppm 0.99 (s, 9H) 2.48 (s, 3H) 3.38 (d, J = 7.04 Hz, 2H) 4.48-4.72 (m, 1H) 8.11 (br. s., 2H) 9.12 (t, J = 5.67 Hz, 1H). MS: 227 (M + H) |
| Preparation 25: 3-Methyl-N-[(5- methyl-1,2,4- oxadiazol-3- yl)methyl]-L- valinamide trifluoroacetate | 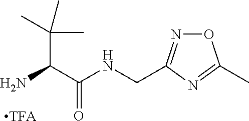 | 1H NMR (400 MHz, DMSO-d₆) δ ppm 0.99 (s, 9H) 2.58 (s, 3H) 3.51 (s, 2H) 4.33-4.63 (m, 1H) 8.09 (br. s., 2H) 9.03 (t, J = 5.67 Hz, 1H). MS: 227 (M + H) |
| Preparation 26: N-[(4- Hydroxytetrahydro- 2H-pyran-4- yl)methyl]-3- methyl-L- valinamide trifluoroacetate | 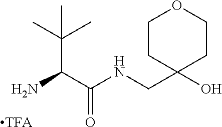 | 1H NMR (400 MHz, DMSO-d₆) δ ppm 0.99 (s, 9H) 1.34-1.46 (m, 2H) 1.49-1.60 (m, 2H) 3.02-3.29 (m, 1H) 3.55-3.66 (m, 6H) 4.57 (br. s., 1H) 8.03 (br. s., 2H) 8.28 (t, J = 5.86 Hz, 1H). MS: 245 (M + H) |

### Preparation 27: (S)-5-((2-amino-3,3-dimethylbutanamido)methyl)-1,3,4-oxadiazole-2-carboxamide, trifluoroacetate salt

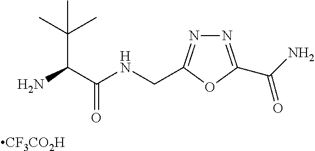

### Step 2: (S)-Tert-butyl 1-((5-carbamoyl-1,3,4-oxadiazol-2-yl)methylamino)-3,3-dimethyl-1-oxobutan-2-ylcarbamate

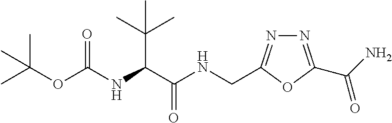

(S)-ethyl 5-((2-(tert-butoxycarbonyl)-3,3-dimethylbutanamido)methyl)-1,3,4-oxadiazole-2-carboxylate (5.72 g, 14.9 mmol) was dissolved into methanol (20 mL) and 2N ammonia in methanol (15 mL) was added. The solution was stirred at ambient temperature for one hour. The solution was concentrated in vacuo to provide the desired material as a white foam (quantitative yield); ¹H NMR (400 MHz, DMSO-d₆) δ ppm 0.90 (s, 9H) 1.38 (s, 9H) 3.89 (d, J=9.77 Hz, 2H) 4.46-4.66 (m, 1H) 6.52 (d, J=8.99 Hz, 1H) 8.18 (s, 1H) 8.56 (s, 1H) 8.73 (t, J=4.89 Hz, 1H)

### Step 3: (S)-5-(2-Amino-3,3-dimethylbutanamido)methyl)-1,3,4-oxadiazole-2-carboxamide, trifluoroacetate salt

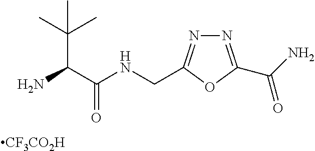

The (S)-tert-butyl 1-((5-carbamoyl-1,3,4-oxadiazol-2-yl)methylamino)-3,3-dimethyl-1-oxobutan-2-ylcarbamate (5.7 g, 14.9 mmol) was dissolved into dichloromethane (20 mL) and trifluoroacetic acid (10 mL) was added. The solution was stirred at ambient temperature for one hour. Concentration in vacuo followed by titration with diethyl ether provided the desired compound as a white solid (5.21 g, 95% yield).

¹H NMR (400 MHz, DMSO-d₆) δ ppm 1.00 (s, 9H) 3.54 (d, J=5.47 Hz, 2H) 4.62-4.78 (m, 1H) 8.11 (br. s., 2H) 8.23 (s, 1H) 8.61 (s, 1H) 9.21 (t, 1H)

### Preparation 28: 5-((S)-1-Amino-2,2-dimethylpropyl)-[1,3,4]oxadiazol-2-ylamine dihydrochloride

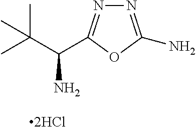

### Step 1: ((S)-1-Hydrazinocarbonyl-2,2-dimethylpropyl)carbamic acid tert-butyl ester

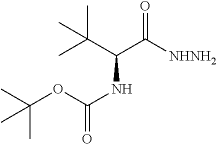

To a solution of N-Boc-L-tert-leucine (2.0 g, 8.647 mmol) in dry THF (20 mL), N,N-carbonyl diimidazole (CDI) (1.54 g, 9.511 mmol) was added and stirred at room temperature under nitrogen atmosphere for 1.5 h. Hydrazine hydrate (1.3 ml, 26.6 mmol) was then added to it and stirring was continued for 18 h at room temperature. On completion of reaction (monitored by TLC, R_{f}=0.3; solvent system 40% ethyl acetate in hexane), THF was evaporated up to dryness and the residual mass dissolved in 1,4-dioxane (50 mL) and filtered. The filtrate was concentrated under reduced pressure and the residual mass (as white sticky material) was again dissolved in DCM. The solution was washed with distilled water, brine, dried over anhydrous Na₂SO₄ and concentrated under reduced pressure to afford desired product ((S)-1-hydrazinocarbonyl-2,2-dimethylpropyl)carbamic acid tert-butyl ester (2.3 g) as gummy sticky mass contaminated with imidazole.

¹H NMR (400 MHz, DMSO-d₆) δ: 0.87 (s, 9H), 1.37 (s, 9H), 3.80 (d, J=9.6 Hz, 1H), 6.35 (d, J=9.6 Hz, 1H), 9.10 (s, 1H)+Imidazole: 7.01 (s, 2H), 7.63 (s, 1H). ¹H NMR (400 MHz, DMSO-d₆-D₂O exchange) δ: 0.88 (s, 9H), 1.35 (s, 9H), 3.77 (s, (1H), +Imidazole: 7.01 (2H, 7.65 (s, 1H). FIA-MS: 246.3 [M+H]⁺, 268.3 [M+H+Na]⁺.

### Step 2: [1-(5 Amino-[1,3,4]oxadiazol-2-yl)-(S)-2,2-dimethylpropyl]carbamic acid tert-butyl ester

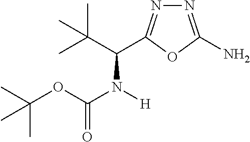

To a clear solution of ((S)-1-hydrazinocarbonyl-2,2-dimethylpropyl)carbamic acid tert-butyl ester (1.5 g, 6.117 mmol) in 1,4-dioxane (50 mL), a solution of NaHCO₃ (0.515 g, 6.117 mmol) in distilled water (15 mL) was added to form a white suspension. Cyanogen bromide (0.65 g, 6.117 mmol) was added portion wise to the reaction mixture and stirred for 18 h at room temperature. On completion of reaction (monitored by TLC, R_{f}=0.5; solvent system 50% ethyl acetate in hexane), the dioxane was evaporated under reduced pressure and ethyl acetate (100 mL) was added. This solution was then washed twice with distilled water (2×100 mL), brine, dried over anhydrous Na₂SO₄ and concentrated under reduced pressure. The residual mass obtained was washed with hexane to afford desired product [1-(5-amino-[1,3,4]oxadiazol-2-yl)-(S)-2,2-dimethylpropyl]carbamic acid tert-butyl ester (0.7 g, yield 42%) as off white solid.

¹H NMR (400 MHz, CDCl₃) δ: 1.01 (s, 9H), 1.27 (s, 9H), 4.65 (d, J=9.6 Hz, 1H), 5.44 (d, J=8.4 Hz, 1H), 8.92 (br s, 2H). MS, 271.4 [M+H]⁺.

### Step 3: 5-((S)-1-Amino-2,2-dimethylpropyl)-[1,3,4]oxadiazol-2-ylamine dihydrochloride

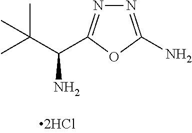

[1-(5-Amino-[1,3,4]oxadiazol-2-yl)-(S)-2,2-dimethylpropyl]carbamic acid tert-butyl ester (4.0 g, 14.81 mmol) was added to 75 mL of 4N HCl in dioxane solution and the solution was stirred at room temperature for 4 h. Evaporation of the reaction mixture under reduced pressure gave 5-((S)-1-amino-2,2-dimethylpropyl)-[1,3,4]oxadiazol-2-ylamine dihydrochloride as white solid (3.5 g, yield 98.59%).

¹H NMR (400 MHz, DMSO-d₆) δ: 0.95 (s, 9H), 4.31 (d, J=5.6 Hz, 1H), 6.34 (br s, 3H), 7.60 (br s, 1H), 8.86 (d, J=4.0 Hz, 3H). LC-MS, 171.1 [M+H]⁺.

### Preparation 29: N-{5-[(1S)-1-amino-2,2-dimethylpropyl]-1,3,4-oxadiazol-2-yl}cyclopropane-carboxamide hydrochloride

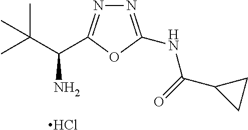

### Step 1: tert-butyl [(1S)-1-{5-[(cyclopropylcarbonyl)amino]-1,3,4-oxadiazol-2-yl}-2,2-dimethylpropyl]carbamate

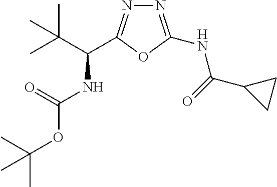

To a mixture of tert-butyl [(1S)-1-(5-amino-1,3,4-oxadiazol-2-yl)-2,2-dimethylpropyl]carbamate (Preparation 28, Step 2, 500 mg, 1.85 mmol) in pyridine (20 ml) was added cyclopropanecarbonyl chloride (202 μl, 2.22 mmol) dropwise. The resultant solution was allowed to stir at ambient temperature. The mixture was poured onto water and extracted with ethyl acetate. The organic layer was concentrated to a residue. Purification was accomplished by SiO₂ chromatography eluting with 0-50% ethyl acetate/heptane, yielding 503 mg (80%) of desired product. ¹H NMR (400 MHz, DMSO-d₆) δ ppm 0.80-0.88 (m, 4H) 0.92 (s, 9H) 1.19-1.29 (m, 1H) 1.35 (s, 9H) 1.79-1.89 (m, 1H) 4.55 (d, J=8.86 Hz, 1H) 7.50 (d, J=8.59 Hz, 1H) 11.77 (s, 1H). FIA-MS: 339.2 [M+H]⁺.

### Step 2: N-{5-[(1S)-1-amino-2,2-dimethylpropyl]-1,3,4-oxadiazol-2-yl}cyclopropane-carboxamide hydrochloride

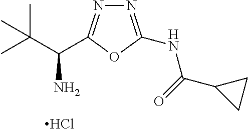

To a solution of tert-butyl [(1S)-1-{5[(cyclopropylcarbonyl)amino]-1,3,4-oxadiazol-2-yl}-2,2-dimethylpropyl]carbamate (502 mg, 1.48 mmol) in dioxane (5 ml) was added HCl (4.0 M in dioxane, 3 ml) at ambient temperature. The resultant mixture was allowed to stir at ambient temperature. The reaction mixture was concentrated to a solid. The solids were suspended in ethyl ether and collected by filtration. The hygroscopic solids were placed in a vacuum oven overnight to dry. Yield=408 mg (94%). ¹H NMR (400 MHz, DMSO-d₆) δ ppm 0.81-0.93 (m, 4H) 0.96-1.02 (m, 9H) 1.92 (t, J=4.57 Hz, 1H) 3.36 (t, J=6.98 Hz, 1H) 4.51 (s, 1H) 5.73 (s, 1H) 8.83 (br. s., 2H) 12.14 (s, 1H). FIA-MS: 237.3 [M+H]⁺.

### Preparation 30: 1-{5-[(1S)-1-Amino-2,2-dimethylpropyl]-1,3,4-oxadiazol-2-yl}urea hydrochloride

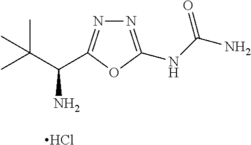

### Step 1: tert-Butyl [(1S)-1-{5-[(aminocarbonyl)amino]-1,3,4-oxadiazol-2-yl}-2,2-dimethylpropyl]carbamate

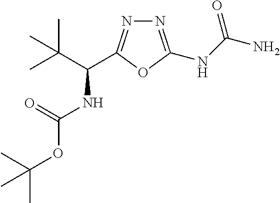

To a stirred solution of tert-butyl [(1S)-1-(5-amino-1,3,4-oxadiazol-2-yl)-2,2-dimethylpropyl]carbamate (Preparation 28, Step 2, 250 mg, 0.9 mmol) in dry THF (5 ml) at 0° C. was added trichloroacetyl isocyanate (240 μl, 2 mmol) slowly, dropwise. The cooling bath was removed after complete addition and reaction mixture allowed to stir at ambient temperature for 1 hour. The mixture was concentrated in vacuo. The residue was dissolved in methanol (3 ml) and purged with ammonia gas for 3 minutes. The resultant mixture was allowed to stir at ambient temperature overnight. The reaction mixture was concentrated by rotary evaporator. The solids were triturated with diethyl ether and collected by filtration yielding 115.5 mg (40%). ¹H NMR (400 MHz, DMSO-d₆) δ ppm 0.96 (s, 9H) 1.38 (s, 9H) 4.54 (d, J=8.99 Hz, 1H) 7.10 (br. s., 2H) 7.51 (d, J=8.79 Hz, 1H) 10.59 (s, 1H). FIA-MS: 314.1 [M+H]⁺.

### Step 2: 1-{5-[(1S)-1-Amino-2,2-dimethylpropyl]-1,3,4-oxadiazol-2-yl}urea hydrochloride

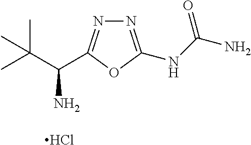

To a solution of tert-butyl [(1S)-1-{5-[(aminocarbonyl)amino]-1,3,4-oxadiazol-2-yl}-2,2-dimethylpropyl]carbamate (115 mg, 0.37 mmol) in dioxane (2 ml) was added HCl (4N in dioxane, 1.5 ml). The resultant mixture was allowed to stir at ambient temperature overnight. The mixture was concentrated under a nitrogen stream and placed on high vacuum yielding 125.4 mg of desired material. ¹H NMR (400 MHz, DMSO-d₆) δ ppm 1.03 (s, 9H) 4.48 (d, J=5.47 Hz, 1H) 7.08 (br. s., 2H) 8.90 (d, J=4.30 Hz, 3H). FIA-MS: 214.2 [M+H]⁺.

### Preparation 31: 5-[(1S)-1-amino-2,2-dimethylpropyl]-1,3,4-oxadiazole-2-carboxamide hydrochloride

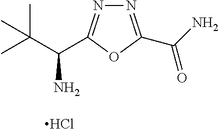

### Step 1: [N′—(S)-2-tert-Butoxycarbonylamino-3,3-dimethyl-butyryl)-hydrazino]-oxo-acetic acid ethyl ester

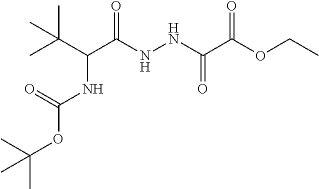

To a solution of ((S)-Hydrazinocarbonyl-2,2-dimethyl-propyl)-carbamic acid tert-butyl ester (Preparation 28, Step 1, 500 mg, 2.0 mmol) and sodium bicarbonate (197 mg, 2.3 mmol) in THF (10 ml) at 0° C. was added ethyloxalyl chloride (239 μl 1, 2.1 mmol) dropwise over 10 minutes. The reaction mixture was allowed to warm to ambient temperature overnight. The reaction mixture was filtered through a cake of Celite eluting with THF. The cloudy filtrate was concentrated to an oily residue. Toluene (˜2 ml) was added and triturated with ethyl ether. The ethereal solution was concentrated to a residue and purified by SiO₂ chromatograhpy eluting with 30-100% ethyl acetate/heptane yielding 653.7 mg (93%). ¹H NMR (400 MHz, DMSO-d₆) δ ppm 0.90 (s, 9H) 1.25 (t, J=7.12 Hz, 3H) 1.35 (s, 9H) 3.91 (d, J=9.67 Hz, 1H) 4.22 (q, 2H) 6.56 (d, J=9.67 Hz, 1H) 10.08 (s, 1H) 10.74 (s, 1H). FIA-MS: 368.2 [M+Na]⁺.

### Step 2: Ethyl 5-{(1S)-1-[(tert-butoxycarbonyl)amino]-2,2-dimethylpropyl}-1,3,4-oxadiazole-2-carboxylate

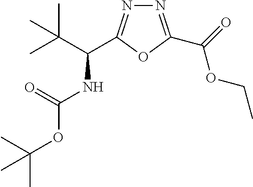

Triethylamine (600 μl, 4.2 mmol) and a solution of [N′—(S)-2-tert-Butoxycarbonylamino-3,3-dimethyl-butyryl)-hydrazino]-oxo-acetic acid ethyl ester (350 mg, 1.0 mmol) in dry dichloromethane (5 ml) was added sequentially to a stirred solution of triphenylphosphine (548 mg, 2.0 mmol) and iodine (851 mg, 2.0 mmol) in dichloromethane (10 ml) at ambient temperature. The reaction was completed in 2 hours. The reaction mixture was extracted (2×30 ml) saturated sodium thiosulfate. The organic layer was concentrated and resultant residue purified by SiO₂ chromatography eluting with 0-75% ethyl acetate/heptane. The oily residue was placed under high vacuum yielding 151.3 mg (46%). ¹H NMR (400 MHz, DMSO-d₆) δ ppm 0.97 (s, 9H) 1.36 (q, 3H) 1.34 (s, 9H) 4.42 (q, J=7.04 Hz, 2H) 4.73 (d, J=8.60 Hz, 1H) 7.73 (d, J=8.60 Hz, 1H). FIA-MS: 350.1 [M+Na]⁺.

### Step 3: tert-butyl {(1S)-1-[5-(aminocarbonyl)-1,3,4-oxadiazol-2-yl]-2,2-dimethylpropyl} carbamate

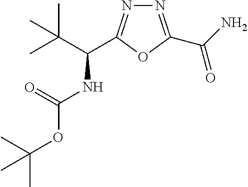

To a solution of ethyl 5-{(1S)-1-[(tert-butoxycarbonyl)amino]-2,2-dimethylpropyl}-1,3,4-oxadiazole-2-carboxylate (150 mg, 0.46 mmol) in ethanol (3 ml) was bubbled ammonia gas for 2 minutes. The vial was sealed and heated at 50° C. overnight. The mixture was concentrated to a residue and dissolved in dichloromethane. The material was purified by SiO₂ chromatography eluting with 0-15% methanol/dichloromethane. The fractions were isolated and concentrated to a residue yielding 123.9 mg (91%). ¹H NMR (400 MHz, DMSO-d₆) δ ppm 0.97 (s, 9H) 1.38 (s, 9H) 4.71 (d, J=8.60 Hz, 1H) 7.67 (d, J=8.60 Hz, 1H) 8.21 (s, 1H) 8.57 (br. s., 1H). FIA-MS: 321.1 [M+Na]⁺.

### Step 4: 5-[(1S)-1-amino-2,2-dimethylpropyl]-1,3,4-oxadiazole-2-carboxamide hydrochloride

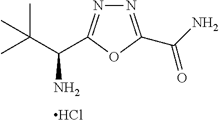

To a solution of tert-butyl {(1S)-1-[5-(aminocarbonyl)-1,3,4-oxadiazol-2-yl]-2,2-dimethylpropyl}carbamate (120 mg, 0.40 mmol) in dioxane (2 ml) was added 4N HCl in dioxane (1 ml). The resultant mixture was stirred at ambient temperature overnight. The reaction mixture was concentrated to a residue. The residue was triturated with ethyl ether and collected by filtration yielding 72.0 mg (76%). ¹H NMR (400 MHz, DMSO-d₆) δ ppm 1.04 (s, 9H) 3.42 (br. s., 1H) 8.33 (s, 1H) 8.71 (s, 1H) 8.92 (br. s., 3H). FIA-MS: 199.1 [M+H]⁺.

### Preparation 32: (1S)-2,2-dimethyl-1-(2H-tetrazol-5-yl)propan-1-amine hydrochloride

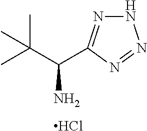

### Step 1: Benzyl [(1S)-1-cyano-2,2-dimethylpropyl]carbamate

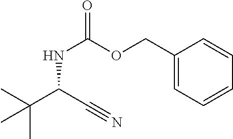

To a solution of benzyl [(1S)-1-(aminocarbonyl)-2,2-dimethylpropyl]carbamate (Preparation 1, Step 1, 2.8 g, 10.9 mmol) in pyridine (25 ml) was added phosphorus oxychloride (1.2 ml, 2.0 g, 13.1 mmol) as a solution in dichloromethane (15 ml), dropwise at −10° C. The resultant mixture stirred for 3 hours. The reaction mixture was poured over ice water (˜100 ml). The layers were separated and organic extracted 1×30 ml 1.0 M CuSO₄ solution, 2×50 ml water and 1×50 ml brine. The organic layer was dried over Na₂SO₄ and concentrated in vacuo. The oily residue was purified by SiO₂ chromatography (70 g) eluting 0-10% methanol/dichloromethane. The oil was taken on in subsequent reactions without additional purification and/or characterization. 2.18 g. LC/MS 247.1 (M+H).

### Step 2: Benzyl [(1S)-2,2-dimethyl-1-(2H-tetrazol-5-yl)propyl]carbamate

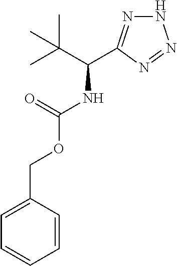

Sodium azide (633 mg, 9.7 mmol) and ammonium chloride (544 mg, 10.2 mmol) were added simultaneously to a solution of benzyl [(1S)-1-cyano-2,2-dimethylpropyl]carbamate (2.2 g, 8.8 mmol) in DMF (35 ml). The resultant reaction mixture was heated to 95° C. for 3 hours. Additional sodium azide (633 mg, 9.7 mmol) and NH₄Cl (544 mg, 10.2 mmol) was added and reaction heated to 95° C. The incomplete reaction mixture was cooled to ambient temperature and quenched by pouring over ice water (˜100 ml). The solution's pH was adjusted to 2 with 4 N HCl. The acidic solution was extracted 3×30 ml CH₂Cl₂. The organic washes were washed with brine (1×30 ml) and dried over MgSO₄. Purification was accomplished by SiO₂ chromatography (Flashmaster 70 g) eluting 10-60% ethyl acetate/hexanes. 646.7 mg, 25% yield. ¹H NMR (400 MHz, DMSO-d₆) δ ppm 0.89 (s, 10H) 4.77 (d, J=8.59 Hz, 1H) 4.99 (d, J=7.25 Hz, 2H) 7.22-7.35 (m, 5H) 7.90 (d, J=8.59 Hz, 1H). LC/MS 290.1 (M+H).

### Step 3: (1S)-2,2-dimethyl-1-(2H-tetrazol-5-yl)propan-1-amine hydrochloride

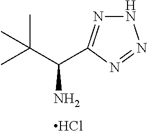

The 5% palladium/charcoal catalyst (20 mg) was added to the dry benzyl [(1S)-2,2-dimethyl-1-(2H-tetrazol-5-yl)propyl]carbamate (600 mg, 2.1 mmol) in a round bottomed flask. To the flask was added methanol (10 ml) under a nitrogen atmosphere. The atmosphere was escaped and purged with hydrogen twice before affixing a hydrogen balloon to the flask. The reaction was maintained at atmospheric pressure overnight at ambient temperature. The reaction mixture was purged with nitrogen gas and filtered through a cake of Celite. The Celite was washed with methanol and filtrate concentrated to a pale tan solid. 320.1 mg, 99% yield. ¹H NMR (400 MHz, DMSO-d₆) δ ppm 0.90 (s, 10H) 4.13 (s, 1H) 7.99 (br. s., 2H). LC/MS 156.1 (M+H).

The following Examples were synthesized according to the general procedures used in the representative Examples and representative Preparations described above.

| Example | Structure | | MS |
|---|---|---|---|
| No. | IUPAC Name | ¹H NMR | (M + H) |
| 13 | 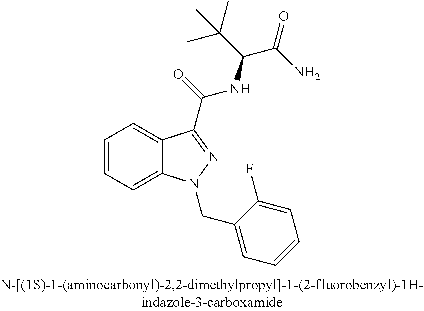 | ¹H NMR (400 MHz, DMSO-d6) δ ppm 1.05 (s, 7H) 1.30 (br. s., 1H) 2.60 (s, 1H) 4.52 (d, J = 9.52 Hz, 1H) 5.90 (s, 2H) 7.22 (d, J = 5.12 Hz, 1H) 7.20 (br. s., 0H) 7.23-7.33 (m, 4H) 7.28 (d, J = 8.05 Hz, 0H) 7.34-7.40 (m, 1H) 7.54 (t, J = 7.69 Hz, 1H) 7.62 (d, J = 9.52 Hz, 1H) 7.73 (br. s., 1H) 7.82 (d, J = 8.05 Hz, 1H) 8.25 (d, J = 8.78 Hz, 1H) | 383 |
| 14 | 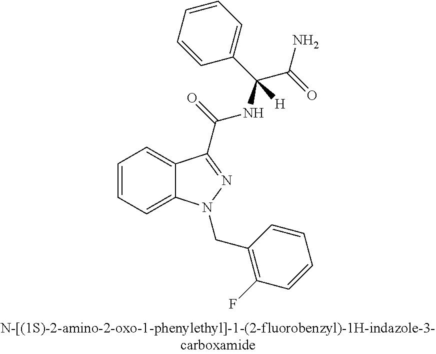 | ¹H NMR (400 MHz, DMSO-d6) δ ppm 8.24 (1H, d, J = 7.7 Hz), 8.14 (1H, d, J = 8.4 Hz), 7.84 (1H, br. s.), 7.76 (1H, d, J = 8.4 Hz), 7.49 (3H, d, J = 7.3 Hz), 7.33-7.39 (4H, m), 7.26-7.32 (2H, m), 7.16 (2H, d, J = 4.8 Hz), 5.85 (3H, s), 5.60 (1H, d, J = 7.7 Hz) | 403 |
| 15 | 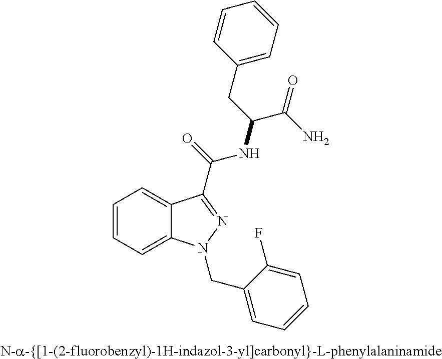 | ¹H NMR (400 MHz, DMSO-d6) δ ppm 3.04-3.11 (m, 1H) 3.12-3.18 (m, 1H) 4.68-4.78 (m, 1H) 5.80 (s, 3H) 7.14-7.26 (m, 9H) 7.34-7.41 (m, 1H) 7.46 (t, J = 7.69 Hz, 1H) 7.60 (br. s., 1H) 7.75 (d, J = 8.42 Hz, 1H) 7.89 (d, J = 8.05 Hz, 1H) 8.13 (d, J = 8.05 Hz, 1H) | 417 |
| 16 | 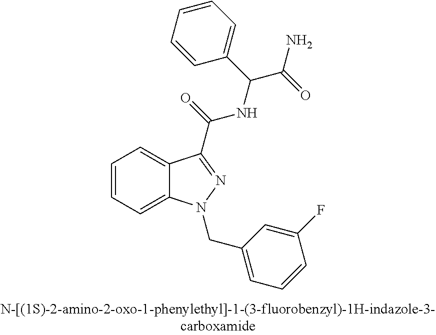 | ¹H NMR (400 MHz, DMSO-d6) δ ppm 8.30 (1H, d, J = 7.3 Hz), 8.14 (1H, d, J = 8.1 Hz), 7.84 (1H, br. s.), 7.78 (1H, d, J = 8.4 Hz), 7.43-7.53 (2H, m), 7.50 (2H, d, J = 7.3 Hz), 7.37 (4H, t, J = 7.1 Hz), 7.29 (2H, t, J = 7.5 Hz), 7.06 (1H, d, J = 7.3 Hz), 7.12 (1H, d, J = 9.1 Hz), 5.83 (2H, s), 5.61 (1H, d, J = 7.3 Hz) | 403 |
| 17 | 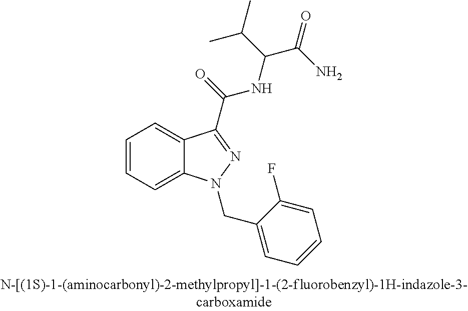 | ¹H NMR (400 MHz, DMSO-d6) δ ppm 0.98 (dd, J = 18.30, 6.59 Hz, 5H) 1.30 (br. s., 1H) 2.16 (dd, J = 13.18, 6.59 Hz, 1H) 2.60 (s, 1H) 4.47 (q, 1H) 5.89 (s, 2H) 7.14-7.25 (m, 2H) 7.30 (t, 1H) 7.36 (t, J = 7.69 Hz, 1H) 7.39-7.48 (m, 1H) 7.53 (t, J = 7.32 Hz, 1H) 7.67 (br. s., 1H) 7.74 (d, J = 8.78 Hz, 1H) 7.81 (d, J = 8.78 Hz, 1H) 8.25 (d, J = 8.05 Hz, 1H) | 369 |
| 18 | 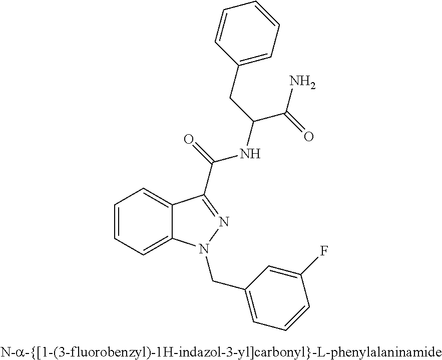 | ¹H NMR (400 MHz, DMSO-d6) δ ppm 8.13 (1H, d, J = 8.4 Hz), 7.99 (1H, d, J = 8.1 Hz), 7.78 (1H, d, J = 8.4 Hz), 7.61 (1H, br. s.), 7.45 (1H, t, J = 7.7 Hz), 7.38 (1H, q), 7.15-7.27 (7H, m), 7.05-7.11 (2H, m), 5.77 (2H, s), 4.72-4.79 (1H, m), 3.14-3.20 (1H, m), 3.05-3.12 (1H, m) | 417 |
| 19 | 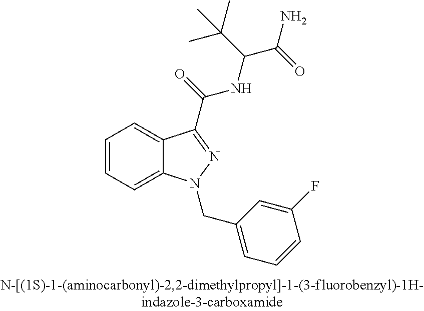 | ¹H NMR (400 MHz, DMSO-d6) δ ppm 8.19 (1H, d, J = 8.1 Hz), 7.78 (1H, d, J = 8.4 Hz), 7.69 (1H, br. s.), 7.61 (1H, d, J = 9.9 Hz), 7.47 (1H, t, J = 7.9 Hz), 7.30 (1H, t, J = 7.5 Hz), 7.28-7.39 (1H, m), 7.21 (1H, br. s.), 7.10 (2H, d, J = 9.1 Hz), 7.05 (1H, d, J = 7.7 Hz), 5.82 (2H, s), 4.47 (1H, d, J = 9.5 Hz), 3.27 (1H, s), 1.00 (9H, s) | 383 |
| 20 | 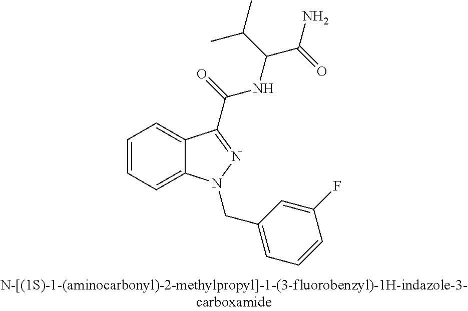 | ¹H NMR (400 MHz, DMSO-d6) δ ppm 8.19 (1H, d, J = 8.1 Hz), 7.77 (2H, t), 7.63 (1H, br. s.), 7.47 (1H, t, J = 7.5 Hz), 7.30 (1H, t, J = 7.5 Hz), 7.28-7.39 (1H, m), 7.17 (1H, br. s.), 7.10 (2H, d, J = 9.1 Hz), 7.05 (1H, d, J = 7.3 Hz), 5.81 (2H, s), 4.42 (1H, dd, J = 8.6, 6.4 Hz), 2.07-2.16 (1H, m), 0.93 (6H, dd, J = 17.0, 6.8 Hz) | 369 |
| 21 | 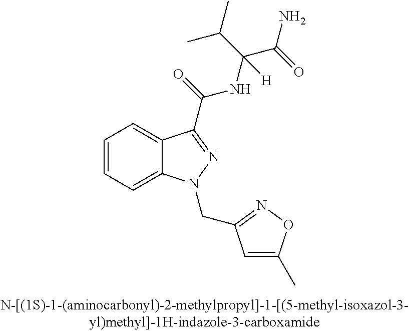 | ¹H NMR (400 MHz, DMSO-d6) δ ppm 8.18 (1H, d, J = 8.1 Hz), 7.72 (1H, d, J = 9.1 Hz), 7.78 (1H, d, J = 8.4 Hz), 7.63 (1H, br. s.), 7.49 (1H, t, J = 7.7 Hz), 7.31 (1H, t, J = 7.5 Hz), 7.18 (1H, br. s.), 6.07 (1H, s), 5.84 (2H, s), 4.41 (1H, t), 2.33 (3H, s), 2.11 (1H, q), 0.93 (6H, dd, J = 17.7, 6.8 Hz) | 356 |
| 22 | 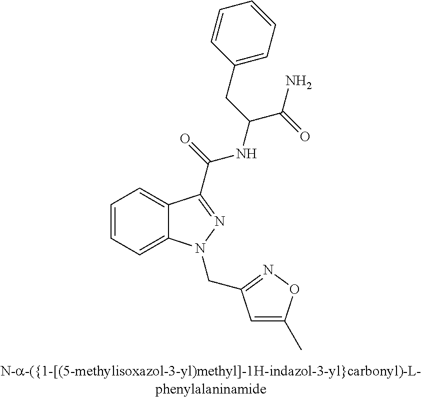 | ¹H NMR (400 MHz, DMSO-d6) δ ppm 8.13 (1H, d, J = 8.1 Hz), 7.97 (1H, d, J = 7.7 Hz), 7.77 (1H, d, J = 8.8 Hz), 7.61 (1H, br. s.), 7.48 (1H, t, J = 7.5 Hz), 7.20-7.31 (5H, m), 7.16 (2H, br. s.), 6.03 (1H, s), 5.80 (2H, s), 4.72-4.79 (1H, m), 3.13-3.20 (1H, m), 3.05-3.13 (1H, m), 2.34 (3H, s) | 404 |
| 23 | 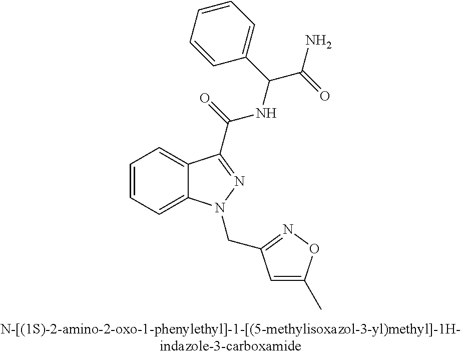 | ¹H NMR (400 MHz, DMSO-d6) δ ppm 8.27 (1H, d, J = 7.7 Hz), 8.13 (1H, d, J = 8.1 Hz), 7.85 (1H, br. s.), 7.78 (1H, d, J = 8.4 Hz), 7.50 (3H, d, J = 7.3 Hz), 7.34-7.40 (3H, m), 7.30 (2H, t, J = 7.1 Hz), 6.08 (1H, s), 5.85 (2H, s), 5.60 (1H, d, J = 7.3 Hz), 2.33 (3H, s) | 390 |
| 24 | 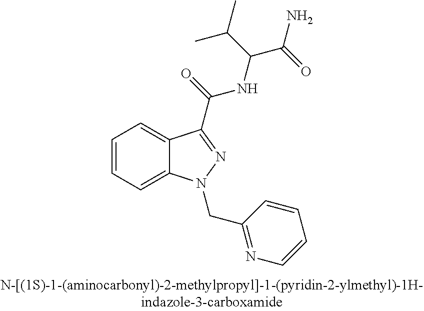 | ¹H NMR (400 MHz, DMSO-d6) δ ppm 8.50 (1H, d, J = 4.0 Hz), 8.19 (1H, d, J = 8.4 Hz), 7.71 (2H, d, J = 8.8 Hz), 7.68-7.78 (1H, m), 7.62 (1H, br. s.), 7.45 (1H, t, J = 7.7 Hz), 7.29 (2H, t, J = 7.7 Hz), 7.12-7.20 (2H, m), 5.88 (2H, s), 4.42 (1H, dd, J = 8.8, 6.6 Hz), 2.05-2.14 (1H, m, J = 13.2, 7.0, 6.6, 6.6 Hz), 0.92 (6H, dd, J = 19.4, 6.6 Hz) | 352 |
| 25 | 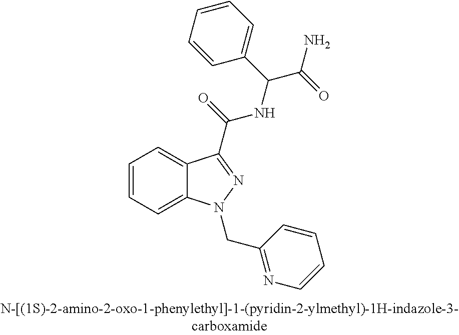 | ¹H NMR (400 MHz, DMSO-d6) δ ppm 8.50 (1H, d, J = 3.7 Hz), 8.25 (1H, d, J = 7.7 Hz), 8.14 (1H, d, J = 8.1 Hz), 7.84 (1H, br. s.), 7.72 (1H, d, J = 8.4 Hz), 7.69-7.80 (1H, m), 7.44 (1H, t, J = 7.9 Hz), 7.49 (2H, d, J = 7.7 Hz), 7.26-7.38 (6H, m), 7.17 (1H, d, J = 7.7 Hz), 5.89 (2H, s), 5.61 (1H, d. J = 7.7 Hz) | 386 |
| 26 | 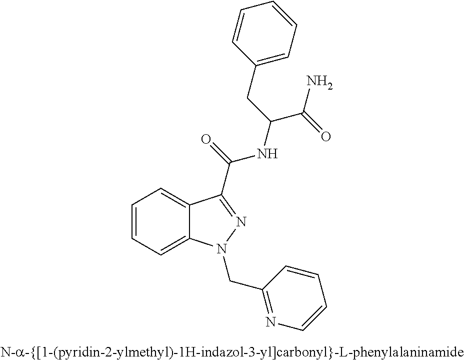 | ¹H NMR (400 MHz, DMSO-d6) δ ppm 8.50 (1H, d, J = 4.4 Hz), 8.14 (1H, d, J = 8.1 Hz), 7.95 (1H, d, J = 8.4 Hz), 7.71 (1H, d, J = 8.8 Hz), 7.76 (2H, t, J = 7.3 Hz), 7.60 (1H, br. s.), 7.43 (1H, t, J = 7.7 Hz), 7.27-7.29 (1H, m), 7.15-7.25 (6H, m), 7.10 (1H, d, J = 7.7 Hz), 5.84 (2H, s), 4.71-4.80 (1H, m), 3.12-3.18 (1H, m), 3.04-3.11 (1H, m) | 400 |
| 27 | 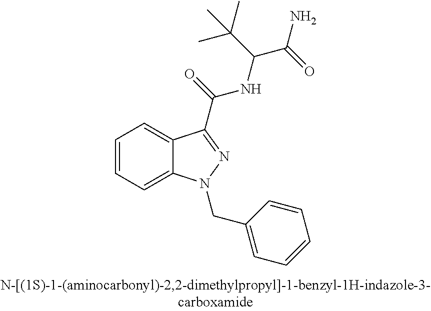 | ¹H NMR (400 MHz, DMSO-d6) δ ppm 8.18 (1H, d, J = 8.4 Hz), 7.75 (2H, dd, J = 17.0, 8.6 Hz), 7.62 (1H, br. s.), 7.46 (1H, t, J = 7.5 Hz), 7.26-7.36 (3H, m), 7.16 (3H, t, J = 8.8 Hz), 5.77 (2H, s), 4.42 (1H, dd, J = 8.8, 6.6 Hz), 2.06-2.16 (1H, m), 0.93 (6H, dd, J = 16.8, 7.0 Hz) | 365 |
| 28 | 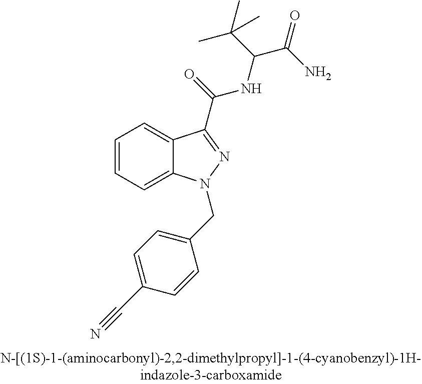 | ¹H NMR (400 MHz, DMSO-d6) δ ppm 0.95 (s, 9H) 4.42 (d, J = 9.52 Hz, 1H) 5.87 (s, 2H) 7.15 (br. s., 1H) 7.23-7.30 (m, 1H) 7.33 (d, J = 8.05 Hz, 2H) 7.43 (t, J = 7.32 Hz, 1H) 7.55 (d, J = 9.52 Hz, 1H) 7.64 (br. s., 1H) 7.71 (d, J = 8.78 Hz, 1H) 7.75 (d, J = 8.78 Hz, 2H) 8.16 (d, J = 8.05 Hz, 1H) | 390 |
| 29 | 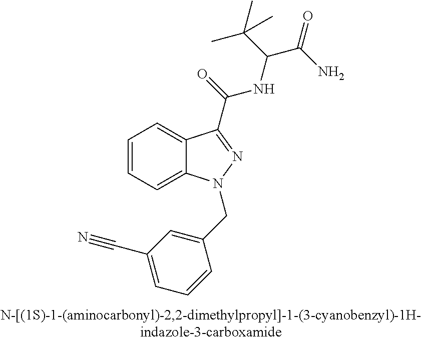 | ¹H NMR (400 MHz, DMSO-d6) δ ppm 0.95 (s, 9H) 4.42 (d, J = 9.52 Hz, 1H) 5.82 (s, 2H) 7.16 (br. s., 1H) 7.27 (t, J = 7.32 Hz, 1H) 7.40-7.46 (m, 1H) 7.47-7.53 (m, 2H) 7.56 (d, J = 9.52 Hz, 1H) 7.63 (br. s., 1H) 7.72 (d, J = 6.59 Hz, 1H) 7.73-7.78 (m, 2H) 8.15 (d, J = 8.05 Hz, 1H) | 390 |
| 30 | 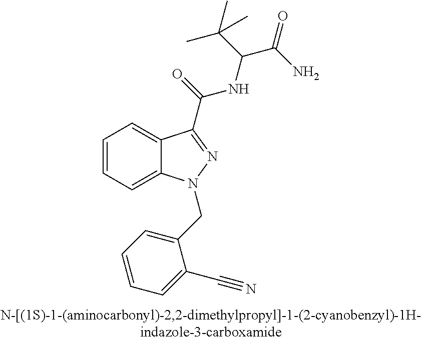 | ¹H NMR (400 MHz, DMSO-d6) δ ppm 0.94 (s, 9H) 4.40 (d, J = 9.52 Hz, 1H) 5.94 (d, J = 2.93 Hz, 2H) 7.07-7.15 (m, 2H) 7.28 (t. J = 7.69 Hz, 1H) 7.41-7.48 (m, 2H) 7.48-7.54 (m, 2H) 7.57-7.65 (m, 2H) 7.74 (d, J = 8.79 Hz, 1H) 7.86 (d, J = 6.59 Hz, 1H) 8.16 (d, J = 8.79 Hz, 1H) | 390 |
| 31 | 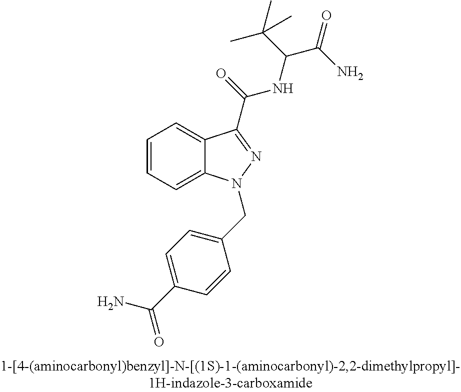 | ¹H NMR (400 MHz, DMSO-d6) δ ppm 0.95 (s, 9H) 4.42 (d, J = 10.25 Hz, 1H) 5.80 (s, 2H) 7.16 (br. s., 1H) 7.19-7.30 (m, 4H) 7.41 (t, J = 7.32 Hz, 1H) 7.57 (d, J = 9.52 Hz, 1H) 7.64 (br. s., 1H) 7.71 (d, J = 8.79 Hz, 1H) 7.76 (d, J = 8.05 Hz, 2H) 7.82 (br. s., 1H) 8.15 (d, J = 8.05 Hz, 1H) | 408 |
| 32 | 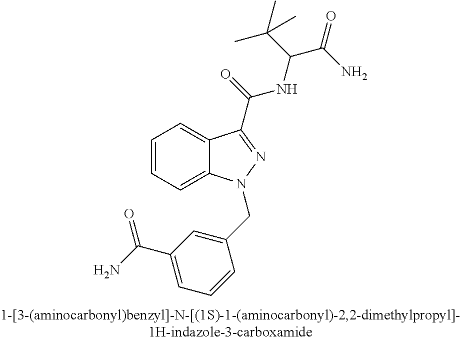 | ¹H NMR (400 MHz, DMSO-d6) δ ppm 0.95 (s, 9H) 4.43 (d, J = 9.52 Hz, 1H) 5.79 (s, 2H) 7.15 (br. s., 1H) 7.25 (t, J = 7.32 Hz, 2H) 7.28-7.32 (m, 1H) 7.36 (t, J = 7.69 Hz, 1H) 7.41 (t, J = 7.32 Hz, 1H) 7.56 (d, J = 10.25 Hz, 1H) 7.64 (br. s., 1H) 7.67-7.76 (m, 2H) 7.79 (s, 1H) 7.88 (br. s., 1H) 8.15 (d, J = 8.05 Hz, 1H) | 408 |
| 33 | 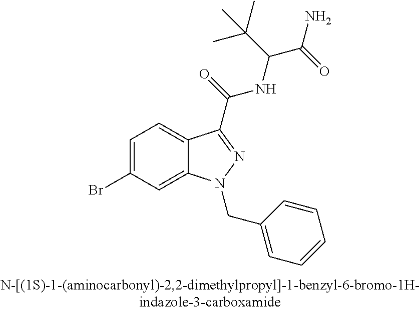 | ¹H NMR (400 MHz, CHLOROFORM-d) δ ppm 1.15 (s, 9H) 4.52 (d, J = 9.22 Hz, 1H) 5.51 (br.s., 1H) 5.58 (s, 2H) 5.88 (br. s., 1H) 7.18-7.22 (m, 2H) 7.29-7.38 (m, 4H) 7.49-7.51 (m, 1H) 7.68 (d, J = 9.56 Hz, 1H) 8.20 (d, J = 8.53 Hz, 1H) | 443, 445 |
| 34 | 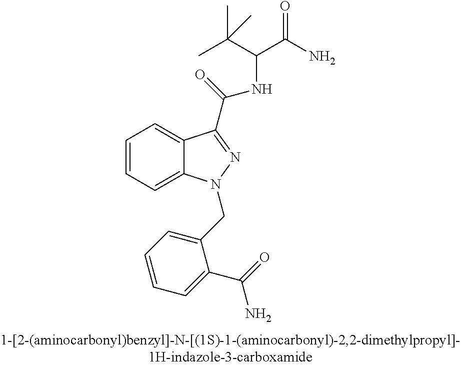 | ¹H NMR (400 MHz, DMSO-d6) δ ppm 0.97 (s, 9H) 4.44 (d, J = 10.25 Hz, 1H) 5.96 (br. s., 2H) 6.73 (d, J = 8.05 Hz, 1H) 7.18 (br. s., 1H) 7.22-7.36 (m, 3H) 7.41 (t, J = 7.69 Hz, 1H) 7.46-7.62 (m, 3H) 7.62-7.71 (m, 2H) 7.96 (br. s., 1H) 8.17 (d, J = 8.05 Hz, 1H) | 408 |
| 35 | 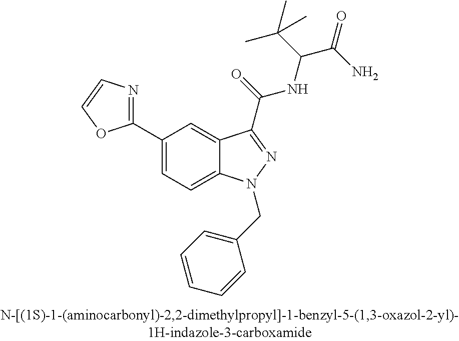 | ¹H NMR (400 MHz, DMSO-d6) δ ppm 0.96 (s, 9H) 4.45 (d, J = 9.52 Hz, 1H) 5.79 (s, 2H) 7.18 (br. s., 1H) 7.20-7.37 (m, 5H) 7.61 (d, J = 9.52 Hz, 1H) 7.67 (br. s., 1H) 7.89 (d, J = 8.79 Hz, 1H) 8.03 (d, J = 9.52 Hz, 1H) 8.17 (s, 1H) 8.77 (s, 1H) | 432 |
| 36 | 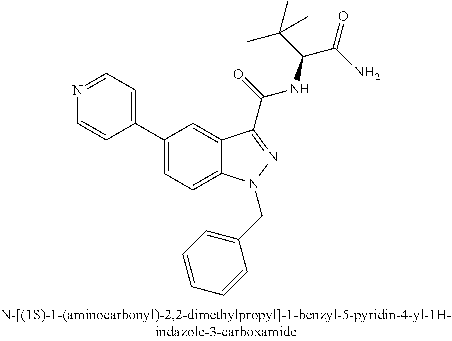 | ¹H NMR (400 MHz, DMSO-d6) δ ppm 0.97 (s, 9H) 4.43 (d, J = 9.52 Hz, 1H) 5.80 (s, 2H) 7.18 (br. s., 1H) 7.21-7.27 (m, 3H) 7.27-7.34 (m, 2H) 7.61 (d, J = 9.52 Hz, 1H) 7.64-7.71 (m, 3H) 7.80-7.86 (m, 1H) 7.85-7.91 (m, 1H) 8.48 (s, 1H) 8.60 (d, J = 5.86 Hz, 2H) | 442 |
| 37 | 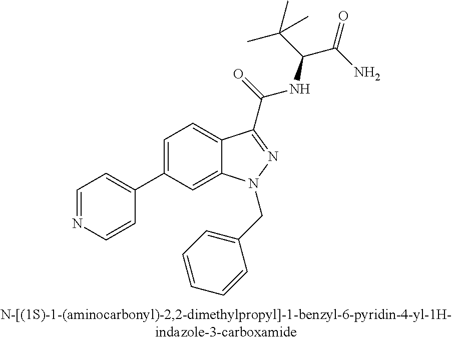 | ¹H NMR (400 MHz, DMSO-d6) δ ppm 0.96 (s, 9H) 4.44 (d, J = 9.52 Hz, 1H) 5.84 (s, 2H) 7.17 (br. s., 1H) 7.21-7.34 (m, 5H) 7.58 (d, J = 9.52 Hz, 1H) 7.65 (br. s., 1H) 7.69 (d, J = 9.52 Hz, 1H) 7.76 (d, J = 5.86 Hz, 2H) 8.19-8.31 (m, 2H) 8.63 (d, J = 5.12 Hz, 2H) | 442 |
| 38 | 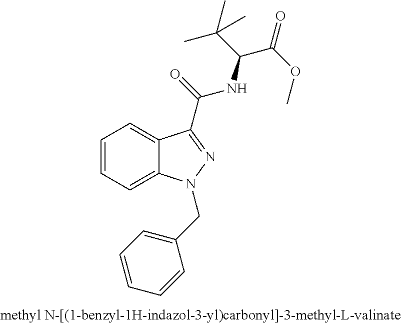 | ¹H NMR (400 MHz, DMSO-d6) δ ppm 0.98 (s, 9H) 3.66 (s, 3H) 4.46 (d, J = 8.78 Hz, 1H) 5.75 (s, 2H) 7.13-7.33 (m, 5H) 7.36-7.48 (m, 2H) 7.63 (d, J = 9.52 Hz, 1H) 7.73 (d, J = 8.05 Hz, 1H) 8.10 (d, J = 8.05 Hz, 1H) | 380 |
| 39 | 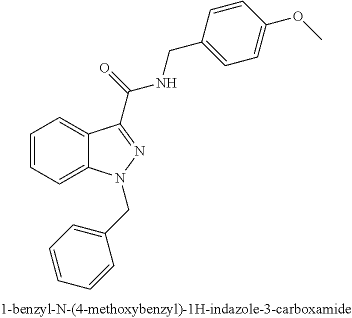 | ¹H NMR (400 MHz, DMSO-d6) δ ppm 3.68 (s, 4H) 4.38 (d, J = 5.86 Hz, 2H) 5.69 (s, 2H) 6.83 (d, J = 8.05 Hz, 2H) 7.17-7.32 (m, 8H) 7.39 (t, J = 7.32 Hz, 1H) 7.70 (d, J = 8.05 Hz, 1H) 8.15 (d, J = 8.05 Hz, 1H) 8.73 (t, J = 6.22 Hz, 1H) | 372 |
| 40 | 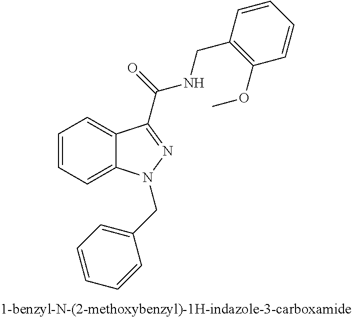 | ¹H NMR (400 MHz, DMSO-d6) δ ppm 3.80 (s, 3H) 4.45 (d, J = 5.86 Hz, 2H) 5.71 (s, 2H) 6.85 (t, J = 7.32 Hz, 1H) 6.95 (d, J = 8.05 Hz, 1H) 7.14-7.31 (m, 7H) 7.35-7.45 (m, 2H) 7.72 (d, J = 8.79 Hz, 1H) 8.14 (d, J = 8.05 Hz, 1H) 8.47-8.56 (m, 1H) | 372 |
| 41 | 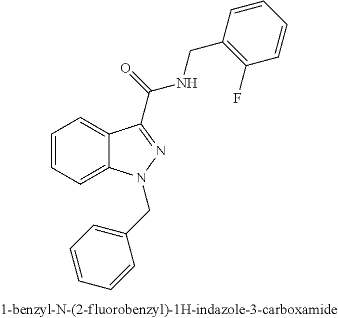 | ¹H NMR (400 MHz, DMSO-d6) δ ppm 4.52 (d, J = 5.86 Hz, 2H) 5.71 (s, 2H) 7.08-7.16 (m, 2H) 7.18-7.32 (m, 6H) 7.33-7.44 (m, 2H) 7.72 (d, J = 8.05 Hz, 1H) 8.14 (d, J = 8.05 Hz, 1H) 8.81 (t, J = 6.22 Hz, 1H) | 360 |
| 42 | 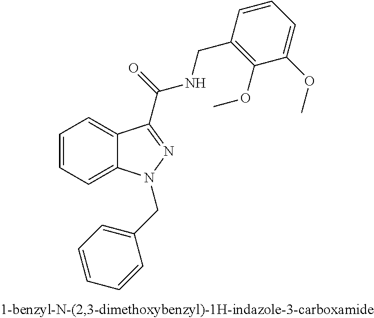 | ¹H NMR (400 MHz, DMSO-d6) δ ppm 3.75 (d, J = 3.66 Hz, 6H) 4.49 (d, J = 6.59 Hz, 2H) 5.71 (s, 2H) 6.87 (t, J = 7.69 Hz, 1H) 6.90 (s, 1H) 6.93-7.01 (m, 1H) 7.18-7.34 (m, 6H) 7.39 (t, J = 7.69 Hz, 1H) 7.71 (d, J = 8.05 Hz, 1H) 8.15 (d, J = 8.05 Hz, 1H) 8.57 (t, J = 6.22 Hz, 1H) | 402 |
| 43 | 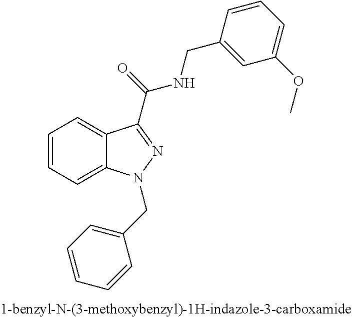 | ¹H NMR (400 MHz, DMSO-d6) δ ppm 3.68 (s, 3H) 4.43 (d, J = 5.86 Hz, 2H) 5.71 (s, 2H) 6.76 (d, J = 9.52 Hz, 1H) 6.89 (d, J = 4.39 Hz, 2H) 7.11-7.32 (m, 5H) 7.39 (t, J = 7.32 Hz, 1H) 7.71 (d, J = 8.79 Hz, 1H) 8.15 (d, J = 8.79 Hz, 1H) 8.80 (t, J = 6.22 Hz, 1H) | 372 |
| 44 | 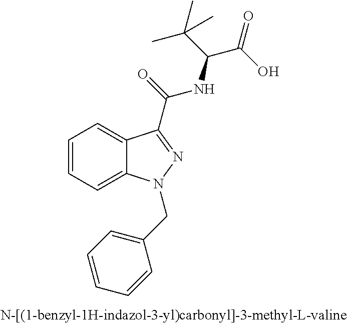 | ¹H NMR (400 MHz, DMSO-d6) δ ppm 0.99 (s, 9H) 4.38 (d, J = 9.52 Hz, 1H) 5.75 (s, 2H) 7.17-7.33 (m, 6H) 7.41 (t, J = 7.69 Hz, 1H) 7.54 (d, J = 9.52 Hz, 1H) 7.72 (d, J = 8.79 Hz, 1H) 8.12 (d, J = 8.05 Hz, 1H) | 366 |
| 45 | 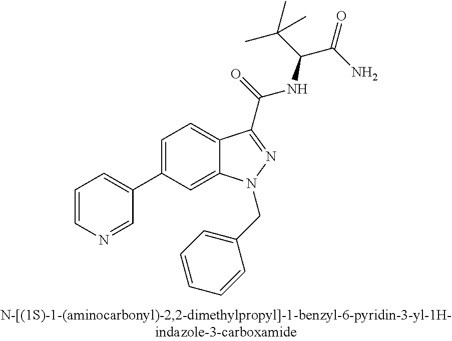 | ¹H NMR (400 MHz, DMSO-d6) δ ppm 0.96 (s, 9H) 4.44 (d, J = 9.52 Hz, 1H) 5.82 (s, 2H) 7.17 (br. s., 1H) 7.24 (d, J = 6.59 Hz, 1H) 7.25-7.33 (m, 4H) 7.48 (dd, J = 8.05, 4.39 Hz, 1H) 7.58 (d, J = 9.52 Hz, 2H) 7.61-7.68 (m, 2H) 8.13 (d, J = 8.05 Hz, 1H) 8.18 (s, 1H) 8.23 (d, J = 8.79 Hz, 1H) 8.52-8.60 (m, 1H) 8.95 (d, J = 2.20 Hz, 1H) | 442 |
| 46 | 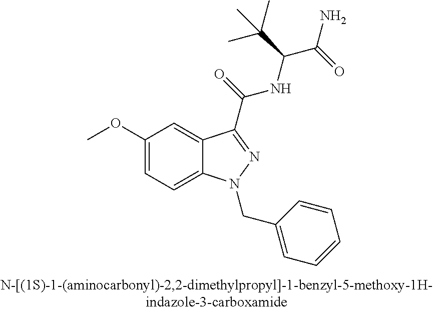 | ¹H NMR (400 MHz, CHLOROFORM-d) δ ppm 1.15 (s, 9H) 3.85 (s, 3H) 4.50 (d, J = 9.40 Hz, 1H) 5.42 (s, 1H) 5.57 (s, 2H) 5.86 (s, 1H) 6.98 (dd, J = 9.13, 2.42 Hz, 1H) 7.15-7.20 (m, 3H) 7.26-7.33 (m, 3H) 7.65 (d, J = 9.40 Hz, 1H) 7.67 (d, J = 2.15 Hz, 1H) | 395 |
| 47 | 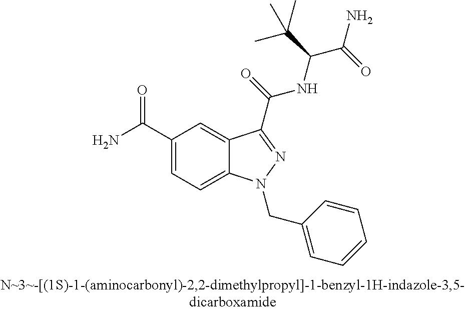 | ¹H NMR (400 MHz, CHLOROFORM-d) δ ppm 1.14 (s, 9H) 4.56 (d, J = 9.22 Hz, 1H) 5.61 (s, 2H) 5.77 (s, 1H) 6.20 (s, 1H) 7.18-7.23 (m, J = 6.49 Hz, 2H) 7.29-7.38 (m, 4H) 7.73 (d, J = 9.22 Hz, 1H) 7.97 (d, J = 8.88 Hz, 1H) 8.71 (s, 1H) 5-CONH2 protons not observed | 408 |
| 48 | 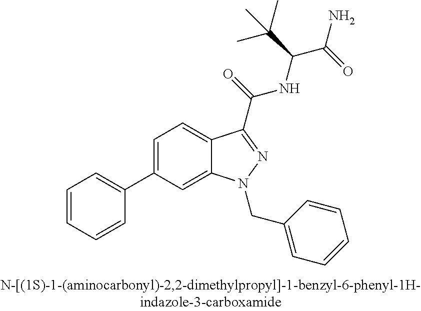 | ¹H NMR (400 MHz, CHLOROFORM-d) δ ppm 1.17 (s, 9H) 4.57 (d, J = 9.22 Hz, 1H) 5.50 (s, 1H) 5.67 (s, 1H) 5.96 (br. s., 1H) 7.22-7.25 (m, 2H) 7.28-7.39 (m, 4H) 7.42-7.48 (m, 3H) 7.51-7.54 (m, 1H) 7.56-7.59 (m, 2H) 7.71 (d, J = 9.22 Hz, 1H) 8.37 (d, J = 8.53 Hz, 1H) | 441 |
| 49 | 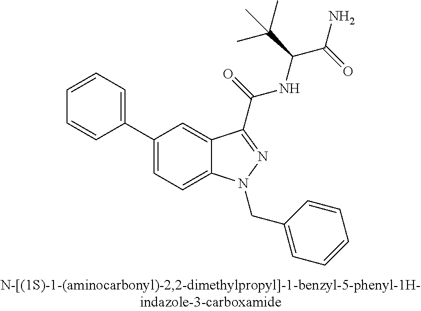 | ¹H NMR (400 MHz, CHLOROFORM-d) δ ppm 1.17 (s, 9H) 4.58 (d, J = 9.22 Hz, 1H) 5.50 (br. s., 1H) 5.65 (s, 2H) 6.00 (br. s., 1H) 7.24 (d, J = 6.14 Hz, 2H) 7.27-7.46 (m, 8H) 7.59-7.67 (m, 3H) 8.56 (d, J = 1.71 Hz, 1H) | 441 |
| 50 | 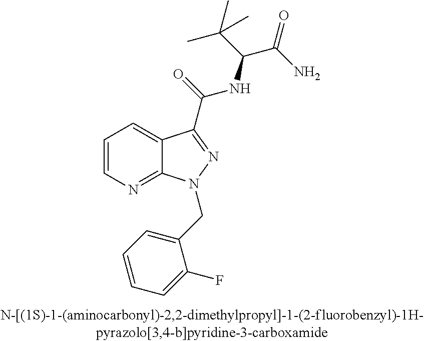 | ¹H NMR (400 MHz, DMSO-d6) δ ppm 0.95 (s, 9H) 2.46 (br. s., 1H) 4.41 (d, J = 9.52 Hz, 1H) 5.82 (s, 2H) 7.12 (s, 1H) 7.17 (br. s., 2H) 7.35 (br. s., 2H) 7.56 (br. s., 1H) 7.64 (br. s., 1H) 8.51 (br. s., 1H) 8.63 (br. s., 1H) | 384 |
| 51 | 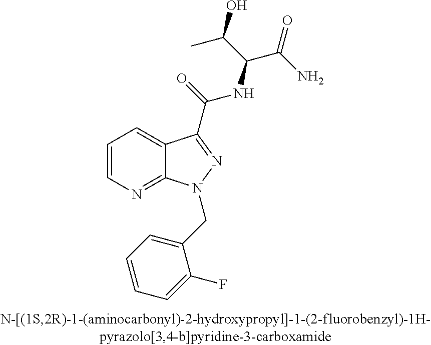 | ¹H NMR (400 MHz, DMSO-d6) δ ppm 1.06 (d, J = 6.22 Hz, 3H) 2.46 (br. s., 1H) 4.12 (dd, J = 9.15, 5.86 Hz, 1H) 4.33 (dd, J = 8.42, 3.29 Hz, 1H) 5.04 (d, J = 5.12 Hz, 1H) 5.82 (d, J = 3.66 Hz, 2H) 7.06-7.17 (m, 2H) 7.12 (t, J = 6.77 Hz, 2H) 7.39 (dt, J = 8.33, 4.44 Hz, 2H) 7.74 (d, J = 8.42 Hz, 1H) 8.53 (d, J = 6.59 Hz, 1H) 8.64 (d, J = 4.39 Hz, 1H) | 372 |
| 52 | 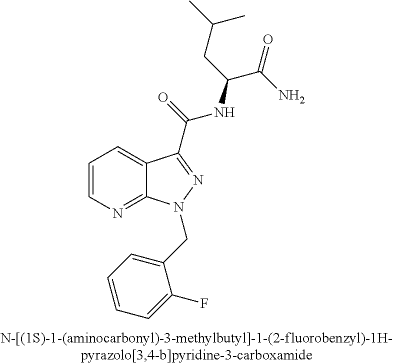 | ¹H NMR (400 MHz, DMSO-d6) δ ppm 0.87 (d, J = 4.39 Hz, 6H) 1.53-1.65 (m, 2H) 1.59 (d, J = 5.49 Hz, 1H) 2.46 (br. s., 2H) 4.50 (t, J = 8.78 Hz, 1H) 5.80 (d, J = 2.56 Hz, 2H) 7.01 (br. s., 1H) 7.10 (d, J = 4.76 Hz, 1H) 7.19 (t, J = 9.15 Hz, 1H) 7.37 (dd, J = 8.05, 4.76 Hz, 1H) 7.46 (br. s., 1H) 8.00 (d, J = 8.78 Hz, 1H) 8.51 (d, J = 1.46 Hz, 1H) 8.62 (d, J = 4.76 Hz, 1H) | 384 |
| 53 | 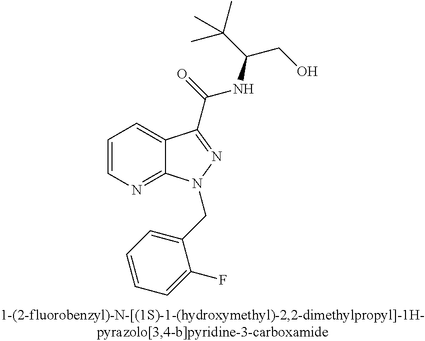 | ¹H NMR (400 MHz, DMSO-d6) δ ppm 0.90 (s, 9H) 2.46 (br. s., 1H) 3.59 (br. s., 2H) 3.86 (br. s., 1H) 4.52 (s, 1H) 5.80 (s, 2H) 7.09 (dd, J = 4.94, 3.11 Hz, 1H) 7.19 (s, 1H) 7.34 (br. s., 2H) 7.60 (br. s., 1H) 8.52 (s, 1H) 8.61 (br. s., 1H) | 371 |
| 54 | 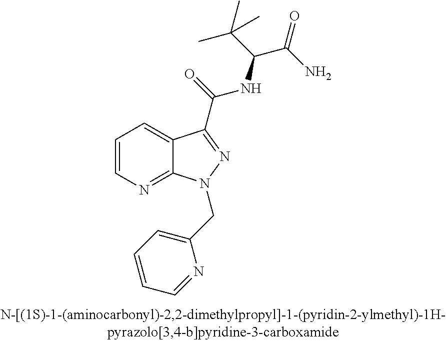 | ¹H NMR (400 MHz, DMSO-d6) δ ppm 0.88 (s, 3H) 0.95 (s, 5H) 1.09 (s. 1H) 2.05-2.15 (m, 2H) 3.25 (d, J = 14.27 Hz, 2H) 3.21 (d, J = 6.59 Hz, 1H) 3.33 (d, J = 6.59 Hz, 1H) 3.36 (br. s., 1H) 3.44 (br. s., 1H) 5.87 (d, J = 6.95 Hz, 1H) 7.16 (d, J = 12.44 Hz, 1H) 7.58 (d, J = 9.52 Hz, 1H) 7.65 (br. s., 1H) 8.59 (br. s., 1H) | 367 |
| 55 | 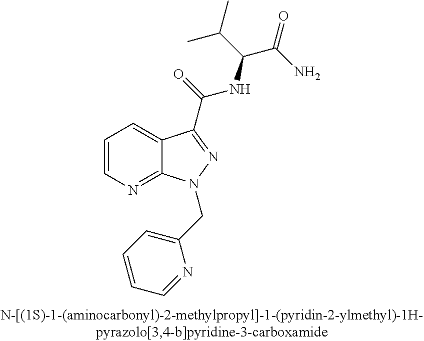 | ¹H NMR (400 MHz, DMSO-d6) δ ppm 0.83-0.93 (m, 6H) 2.02-2.13 (m, 1H) 2.07 (d, J = 6.59 Hz, 1H) 4.37 (dd, J = 8.78, 6.59 Hz, 1H) 5.87 (d, J = 4.39 Hz, 2H) 7.13 (d, J = 6.95 Hz, 2H) 7.26 (d, J = 5.12 Hz, 1H) 7.37 (dd, J = 8.05, 4.76 Hz, 1H) 7.57 (br. s., 1H) 7.71 (t, J = 7.87 Hz, 1H) 7.77 (d, J = 8.78 Hz, 1H) 8.53 (d, J = 8.05 Hz, 1H) 8.60 (d, J = 3.29 Hz, 1H) | 353 |
| 56 | 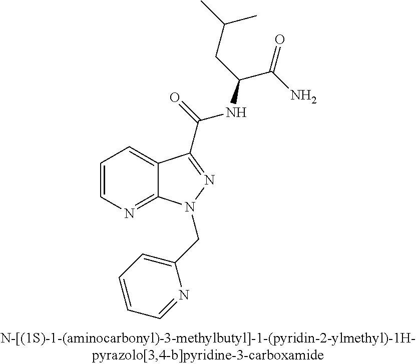 | ¹H NMR (400 MHz, DMSO-d6) δ ppm 0.87 (dd, J = 5.49, 3.29 Hz, 7H) 1.62 (t, J = 11.71 Hz, 1H) 2.09 (d, J = 5.49 Hz, 1H) 2.46 (br. s., 1H) 4.51 (t, J = 8.97 Hz, 1H) 5.86 (d, J = 1.83 Hz, 2H) 7.01 (br. s., 1H) 7.12 (d, J = 7.69 Hz, 1H) 7.26 (d, J = 5.12 Hz, 1H) 7.36 (dd, J = 8.05, 4.39 Hz, 1H) 7.45 (br. s., 1H) 7.71 (t, J = 7.69 Hz, 1H) 8.05 (d, J = 8.78 Hz, 1H) 8.54 (d, J = 8.05 Hz, 1H) 8.59 (d, J = 3.29 Hz, 1H) | 367 |
| 57 | 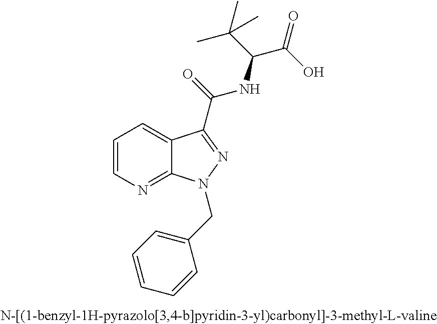 | ¹H NMR (400 MHz, DMSO-d6) δ ppm 1.03 (s, 9H) 4.42 (d, J = 9.52 Hz, 1H) 5.81 (d, J = 4.39 Hz, 2H) 7.25-7.34 (m, 4H) 7.42 (dd, J = 8.05, 4.39 Hz, 1H) 7.65 (s, 1H) 8.54 (d, J = 6.59 Hz, 1H) 8.68 (d, J = 2.93 Hz, 1H) | 481 |
| 58 | 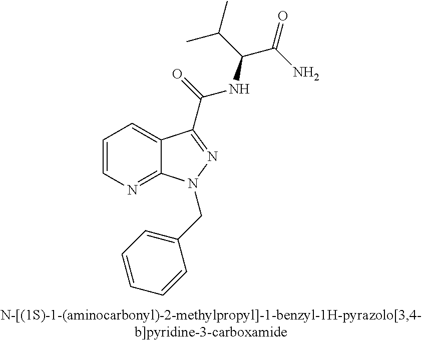 | ¹H NMR (400 MHz, DMSO-d6) δ ppm 0.92 (dd, J = 17.94, 6.59 Hz, 6H) 2.11 (d, J = 6.59 Hz, 1H) 4.41 (dd, J = 8.78, 6.59 Hz, 1H) 5.80 (d, J = 2.20 Hz, 2H) 7.17 (br. s., 1H) 7.25-7.35 (m, 2H) 7.29 (t, J = 7.50 Hz, 3H) 7.41 (dd, J = 8.05, 4.39 Hz, 1H) 7.62 (br. s., 1H) 7.81 (d, J = 8.78 Hz, 1H) 8.56 (d, J = 6.95 Hz, 1H) 8.67 (d, J = 2.93 Hz, 1H) | 352 |
| 59 | 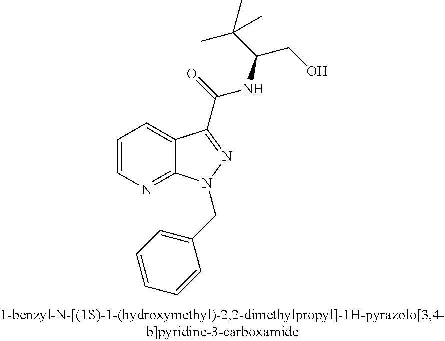 | ¹H NMR (400 MHz, DMSO-d6) δ ppm 0.95 (s, 9H) 3.63 (d, J = 5.86 Hz, 2H) 3.89 (br. s., 1H) 4.56 (s, 1H) 5.79 (d, J = 2.56 Hz, 2H) 7.25-7.34 (m, 4H) 7.68 (d, J = 9.88 Hz, 1H) 8.31-8.96 (m, 1H) | 353 |
| 60 | 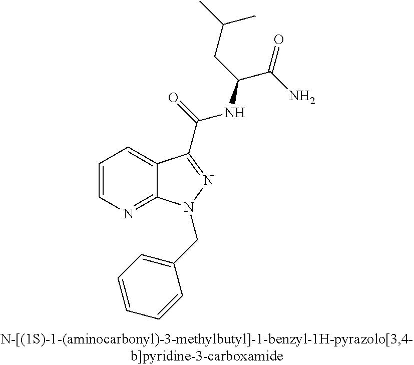 | ¹H NMR (400 MHz, DMSO-d6) δ ppm 0.91 (d, J = 5.49 Hz, 7H) 1.58-1.70 (m, 2H) 1.64 (d, J = 6.22 Hz, 1H) 4.54 (t, J = 8.97 Hz, 1H) 5.79 (s, 2H) 7.04 (br. s., 1H) 7.25-7.35 (m, 1H) 7.29 (t, J = 7.32 Hz, 3H) 7.40 (dd, J = 8.05, 4.39 Hz, 1H) 7.50 (br. s., 1H) 8.08 (d, J = 8.42 Hz, 1H) 8.56 (d, J = 6.95 Hz, 1H) 8.67 (d, J = 3.29 Hz, 1H) | 366 |
| 61 | 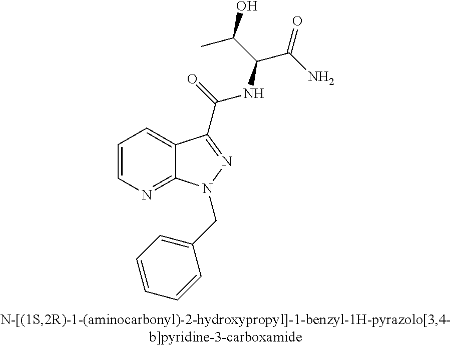 | ¹H NMR (400 MHz, DMSO-d6) δ ppm 1.10 (d, J = 6.59 Hz, 3H) 4.16 (dd, J = 9.52, 5.49 Hz, 1H) 4.37 (dd, J = 8.42, 3.29 Hz, 1H) 5.08 (d, J = 5.12 Hz, 1H) 5.81 (s, 2H) 7.11 (br. s., 1H) 7.26-7.36 (m, 2H) 7.30 (t, J = 5.67 Hz, 3H) 7.43 (dd, J = 12.63, 7.87 Hz, 2H) 7.80 (d, J = 8.42 Hz, 1H) 8.57 (d, J = 6.95 Hz, 1H) 8.68 (d, J = 3.29 Hz, 1H) | 354 |
| 62 | 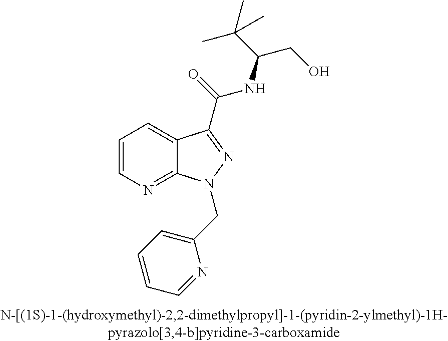 | ¹H NMR (400 MHz, DMSO-d6) δ ppm 0.94 (s, 9H) 3.57-3.67 (m, 2H) 3.82-3.98 (m, 1H) 4.55 (t, J = 5.12 Hz, 1H) 5.90 (d, J = 4.76 Hz, 2H) 7.40 (dd, J = 7.87, 4.58 Hz, 1H) 7.69 (d, J = 9.52 Hz, 1H) 7.72-7.78 (m, 1H) 8.55-8.65 (m, 2H) | 354 |
| 63 | 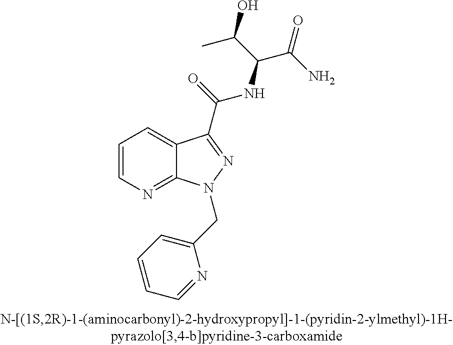 | ¹H NMR (400 MHz, DMSO-d6) δ ppm 1.10 (d, J = 6.22 Hz, 4H) 4.15 (d, J = 3.66 Hz, 1H) 4.37 (dd, J = 8.60, 3.48 Hz, 1H) 5.07 (d, J = 5.49 Hz, 1H) 5.91 (s, 2H) 7.11 (br. s., 1H) 7.18 (d, J = 7.32 Hz, 1H) 7.30 (dd, J = 6.04, 4.58 Hz, 1H) 7.42 (dd, J = 8.05, 4.39 Hz, 2H) 7.80 (d, J = 8.78 Hz, 1H) 7.73-7.82 (m, 1H) 8.58 (d, J = 7.69 Hz, 1H) 8.64 (d, J = 4.03 Hz, 1H) | 355 |
| 64 | 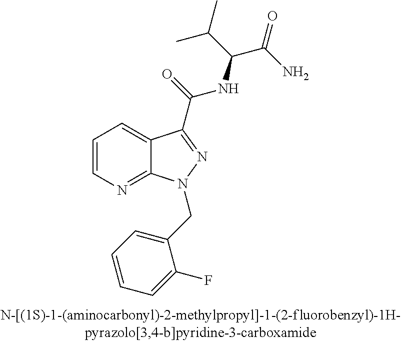 | ¹H NMR (400 MHz, DMSO-d6) δ ppm 0.88 (dd, J = 18.85, 6.77 Hz, 6H) 2.07 (d, J = 6.95 Hz, 1H) 2.46 (br. s., 1H) 4.37 (dd, J = 8.78, 6.22 Hz, 1H) 5.81 (d, J = 2.20 Hz, 2H) 7.08-7.19 (m, 1H) 7.12 (d, J = 5.86 Hz, 2H) 7.29-7.40 (m, 2H) 7.57 (br. s., 1H) 7.73 (d, J = 8.78 Hz, 1H) 8.52 (d, J = 6.59 Hz, 1H) 8.63 (d, J = 2.93 Hz, 1H) | 370 |
| 65 | 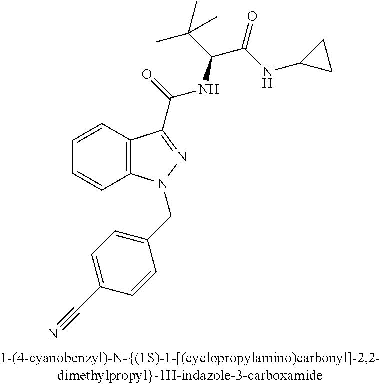 | ¹H NMR (400 MHz, DMSO-d6) δ ppm 0.35-0.45 (m, 2H) 0.58-0.66 (m, 2H) 0.94 (s, 9H) 2.65 (dd, J = 7.32, 3.66 Hz, 1H) 4.39 (d, J = 9.52 Hz, 1H) 5.92 (s, 2H) 7.28-7.37 (m, 3H) 7.47 (t, J = 7.32 Hz, 1H) 7.57 (d, J = 9.88 Hz, 1H) 7.77 (d, J = 8.42 Hz, 1H) 7.80 (d, J = 8.05 Hz, 2H) 8.18 (d, J = 8.42 Hz, 1H) 8.35 (d, J = 4.39 Hz, 1H) | 430 |
| 66 | 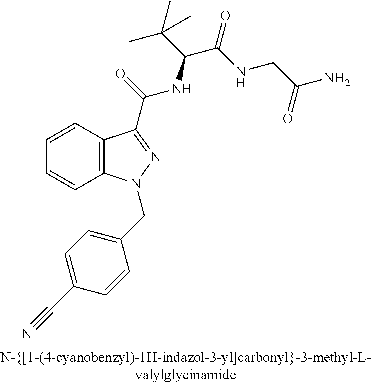 | ¹H NMR (400 MHz, DMSO-d6) δ ppm 0.95 (s, 8H) 2.46 (s, 2H) 3.30 (s, 1H) 3.35 (s, 1H) 3.65 (dd, J = 5.86, 1.46 Hz, 2H) 4.50 (d, J = 9.15 Hz, 1H) 5.89 (s, 2H) 6.97 (br. s., 1H) 7.25-7.34 (m, 3H) 7.43 (t, J = 7.69 Hz, 1H) 7.60 (d, J = 9.52 Hz, 1H) 7.73 (d, J = 8.42 Hz, 1H) 7.77 (d, J = 8.42 Hz, 1H) 8.15 (d, J = 8.05 Hz, 1H) 8.45 (t, J = 5.67 Hz, 1H) | 447 |
| 67 | 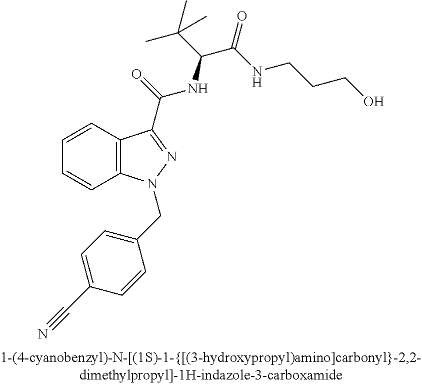 | ¹H NMR (400 MHz, DMSO-d6) δ ppm 0.87-0.96 (m, 8H) 1.53 (td, J = 6.68, 2.38 Hz, 2H) 2.06 (s, 1H) 2.46 (s, 2H) 2.90-2.98 (m, 1H) 2.98-3.06 (m, 1H) 3.12-3.22 (m, 1H) 3.37 (q, J = 6.22 Hz, 2H) 4.38-4.45 (m, 2H) 5.89 (s, 2H) 7.25-7.35 (m, 2H) 7.44 (t, J = 7.69 Hz, 1H) 7.56 (d, J = 9.88 Hz, 1H) 7.73 (d, J = 8.42 Hz, 1H) 7.77 (d, J = 8.05 Hz, 1H) 8.15 (d, J = 8.05 Hz, 1H) 8.24 (t, J = 5.67 Hz, 1H) | 448 |
| 68 | 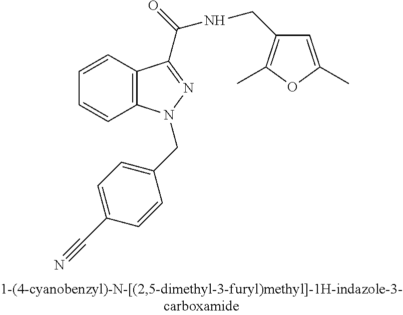 | ¹H NMR (400 MHz, DMSO-d6) δ ppm 2.10 (s, 3H) 2.19 (s, 3H) 2.46 (s, 2H) 4.13 (d, J = 6.22 Hz, 2H) 5.82 (s, 2H) 5.95 (s, 1H) 7.25 (t, J = 7.32 Hz, 1H) 7.31 (d, J = 8.42 Hz, 2H) 7.42 (t, J = 7.32 Hz, 1H) 7.74 (t, J = 8.42 Hz, 3H) 8.17 (d, J = 8.05 Hz, 1H) 8.61 (t, J = 6.04 Hz, 1H) | 385 |
| 69 | 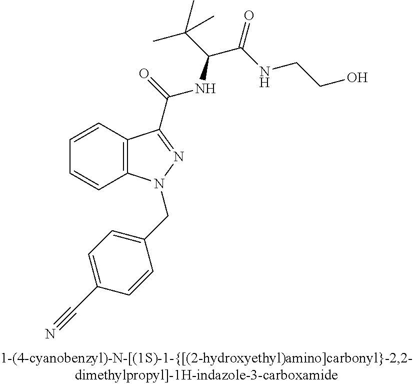 | ¹H NMR (400 MHz, DMSO-d6) δ ppm 0.86-0.96 (m, 9H) 2.06 (s, 1H) 2.46 (s, 2H) 2.93 (d, J = 5.86 Hz, 1H) 3.09 (d, J = 5.86 Hz, 1H) 3.15 (d, J = 5.86 Hz, 1H) 3.18 (d, J = 6.22 Hz, 1H) 3.37 (q, J = 5.86 Hz, 2H) 4.47 (d, J = 9.52 Hz, 1H) 4.64 (t, J = 5.12 Hz, 1H) 5.89 (s, 2H) 7.25-7.34 (m, 2H) 7.43 (t, J = 7.32 Hz, 1H) 7.57 (d, J = 9.88 Hz, 1H) 7.73 (d, J = 8.79 Hz, 1H) 7.77 (d, J = 8.05 Hz, 1H) 8.15 (d, J = 8.05 Hz, 1H) 8.28 (t, J = 5.49 Hz, 1H) | 434 |
| 70 | 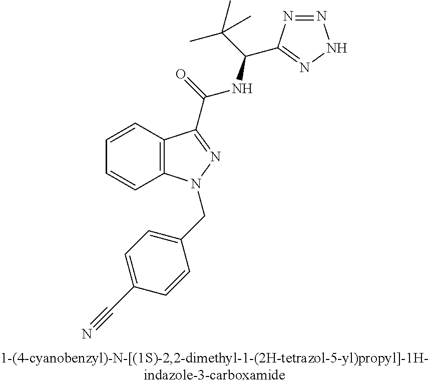 | ¹H NMR (400 MHz, DMSO-d6) δ ppm 0.87-0.96 (m, 6H) 1.61-1.72 (m, 1H) 2.37 (s, 1H) 2.46 (s, 2H) 2.66 (s, 1H) 2.84-2.92 (m, 1H) 2.92-3.02 (m, 1H) 3.21 (br. s., 1H) 3.41 (br. s., 3H) 5.27 (d, J = 9.52 Hz, 1H) 5.90 (s, 1H) 7.26 (t, J = 7.50 Hz, 1H) 7.33 (d, J = 8.42 Hz, 1H) 7.42 (t, J = 7.32 Hz, 1H) 7.71 (d, J = 8.42 Hz, 1H) 7.76 (d, J = 8.42 Hz, 1H) 7.92 (d, J = 9.52 Hz, 1H) | 415 |
| 71 | 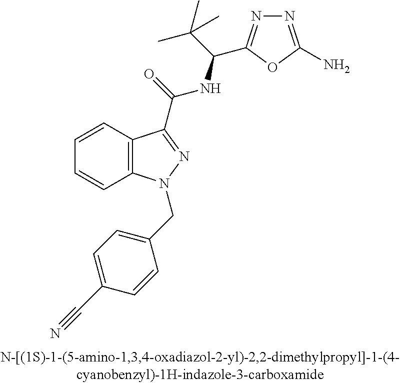 | ¹H NMR (400 MHz, DMSO-d6) δ ppm 0.92 (t, J = 7.14 Hz, 1H) 1.00 (s, 8H) 2.06 (s, 1H) 2.46 (s, 2H) 5.08 (d, J = 9.88 Hz, 1H) 5.88 (s, 2H) 7.01 (s, 2H) 7.28 (t, J = 7.50 Hz, 1H) 7.35 (d, J = 8.05 Hz, 2H) 7.44 (t, J = 7.32 Hz, 1H) 7.73 (d, J = 8.42 Hz, 1H) 7.77 (d, J = 8.42 Hz, 1H) 8.01 (d, J = 9.52 Hz, 1H) 8.12 (d, J = 8.05 Hz, 1H) | 430 |
| 72 | 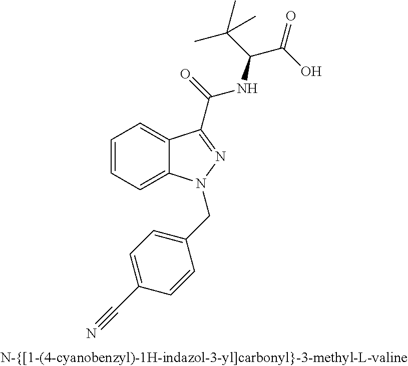 | ¹H NMR (400 MHz, DMSO-d6) δ ppm 1.01 (s, 10H) 2.48 (br. s., 2H) 4.41 (d, J = 9.52 Hz, 1H) 5.90 (s, 2H) 7.30 (t, J = 7.32 Hz, 1H) 7.37 (d, J = 8.05 Hz, 2H) 7.46 (t, J = 7.69 Hz, 1H) 7.57 (d, J = 9.52 Hz, 1H) 7.72-7.82 (m, 3H) 8.16 (d, J = 8.78 Hz, 1H) | 391 |
| 73 | 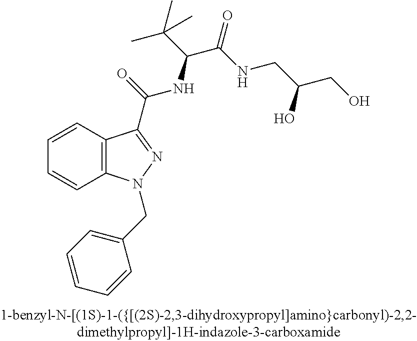 | ¹H NMR (400 MHz, DMSO-d6) δ ppm 0.98 (s, 10H) 2.92-3.03 (m, 1H) 3.24-3.32 (m, 3H) 3.51 (d, J = 6.22 Hz, 1H) 4.52-4.59 (m, 2H) 4.73 (d, J = 4.76 Hz, 1H) 5.80 (s, 2H) 7.23-7.35 (m, 5H) 7.45 (t, J = 7.50 Hz, 1H) 7.64 (d, J = 9.88 Hz, 1H) 7.78 (d, J = 8.79 Hz, 1H) 8.18 (d, J = 8.05 Hz, 1H) 8.28 (t, J = 5.49 Hz, 1H) | 439 |
| 74 | 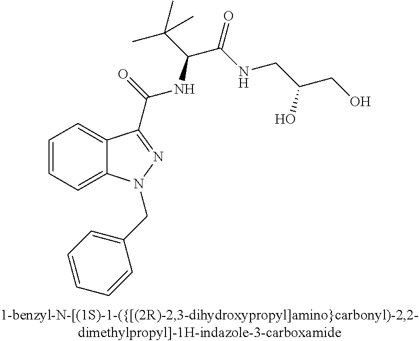 | ¹H NMR (400 MHz, DMSO-d6) δ ppm 0.97 (s, 9H) 3.03-3.12 (m, 1H) 3.19 (dt, J = 13.45, 5.35 Hz, 1H) 3.29 (t, J = 4.94 Hz, 2H) 3.49 (d, J = 4.03 Hz, 1H) 4.55 (d, J = 9.88 Hz, 2H) 4.75 (d, J = 4.39 Hz, 1H) 5.80 (s, 2H) 7.23-7.35 (m, 5H) 7.45 (t, J = 7.50 Hz, 1H) 7.63 (d, J = 9.52 Hz, 1H) 7.78 (d, J = 8.42 Hz, 1H) 8.18 (d, J = 8.05 Hz, 1H) 8.29 (t, J = 5.67 Hz, 1H) | 439 |
| 75 | 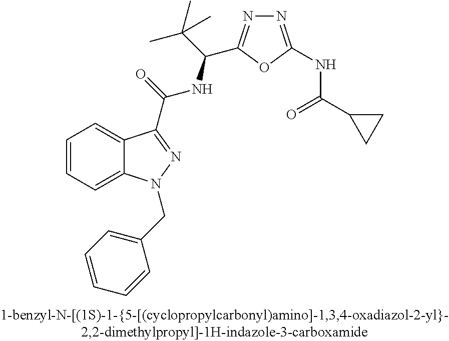 | ¹H NMR (400 MHz, DMSO-d6) δ ppm 0.84-0.92 (m, 4H) 0.97 (s, 1H) 1.02 (s, 3H) 1.07 (s, 7H) 1.88 (br. s., 1H) 5.26 (d, J = 9.15 Hz, 1H) 5.80 (s, 2H) 7.24-7.35 (m, 6H) 7.46 (t, J = 7.69 Hz, 1H) 7.78 (d, J = 8.79 Hz, 1H) 8.11-8.22 (m, 2H) 11.91 (br. s., 1H) | 473 |
| 76 | 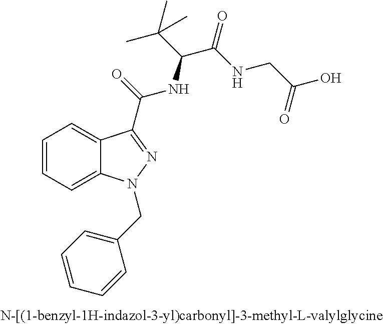 | ¹H NMR (400 MHz, DMSO-d6) δ ppm 1.00 (s, 5H) 2.50 (s, 1H) 3.70-3.90 (m, 2H) 4.56 (d, J = 9.52 Hz, 1H) 5.80 (s, 1H) 7.22-7.36 (m, 4H) 7.46 (t, J = 7.69 Hz, 1H) 7.63 (d, J = 10.25 Hz, 1H) 7.78 (d, J = 8.79 Hz, 1H) 8.18 (d, J = 8.05 Hz, 1H) 8.67 (t, J = 5.86 Hz, 1H) | 423 |
| 77 | 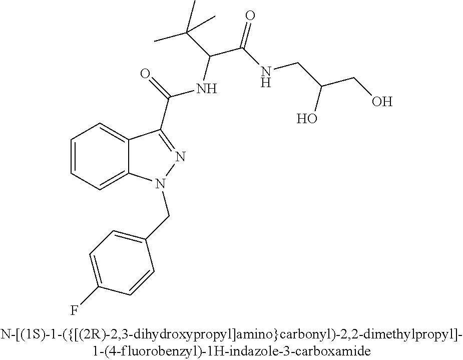 | ¹H NMR (400 MHz, DMSO-d6) δ ppm 0.97 (s, 10H) 3.03-3.12 (m, 1H) 3.14-3.24 (m, 1H) 3.29 (t, J = 5.67 Hz, 2H) 3.49 (d, J = 5.86 Hz, 1H) 4.51-4.58 (m, 2H) 4.75 (d, J = 5.12 Hz, 1H) 5.79 (s, 2H) 7.17 (t, J = 8.97 Hz, 2H) 7.26-7.35 (m, 3H) 7.46 (t, J = 7.69 Hz, 1H) 7.63 (d, J = 9.52 Hz, 1H) 7.80 (d, J = 8.42 Hz, 1H) 8.17 (d, J = 8.05 Hz, 1H) 8.28 (t, J = 5.67 Hz, 1H) | 457 |
| 78 | 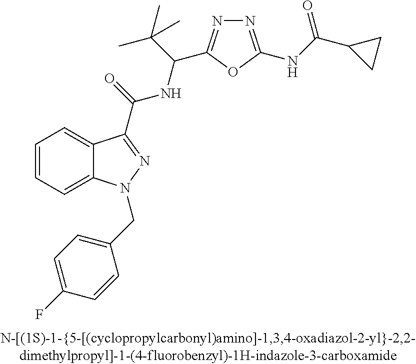 | ¹H NMR (400 MHz, DMSO-d6) δ ppm 0.82-0.92 (m, 5H) 1.02 (s, 3H) 1.07 (s, 7H) 1.87 (d, J = 2.93 Hz, 1H) 5.26 (d, J = 9.15 Hz, 1H) 5.79 (s, 2H) 7.16 (t, J = 8.79 Hz, 2H) 7.27-7.38 (m, 3H) 7.46 (t, J = 7.69 Hz, 1H) 7.80 (d, J = 8.42 Hz, 1H) 8.11-8.22 (m, 2H) 11.92 (br. s., 1H) | 491 |
| 79 | 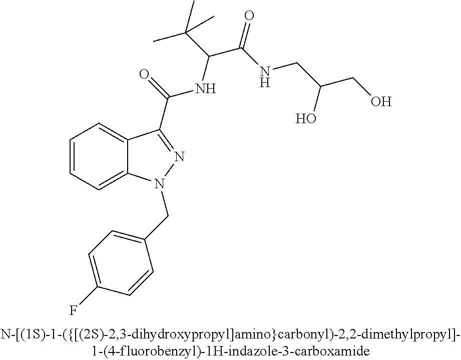 | ¹H NMR (400 MHz, DMSO-d6) δ ppm 0.98 (s, 10H) 2.97 (d, J = 7.32 Hz, 1H) 3.24-3.32 (m, 3H) 3.51 (d, J = 6.22 Hz, 1H) 4.52-4.58 (m, 2H) 4.73 (d, J = 4.76 Hz, 1H) 5.79 (s, 2H) 7.16 (t, J = 8.97 Hz, 2H) 7.26-7.35 (m, 3H) 7.46 (t, J = 7.32 Hz, 1H) 7.63 (d, J = 9.88 Hz, 1H) 7.80 (d, J = 8.42 Hz, 1H) 8.17 (d, J = 8.42 Hz, 1H) 8.27 (t, J = 5.67 Hz, 1H) | 457 |
| 80 | 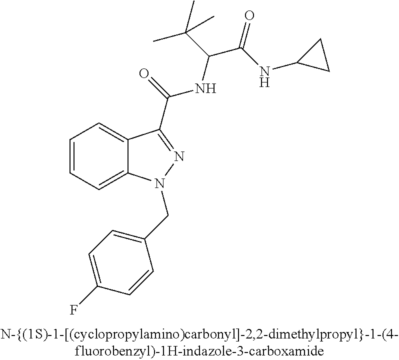 | ¹H NMR (400 MHz, DMSO-d6) δ ppm 0.36-0.46 (m, 2H) 0.57-0.67 (m, 2H) 0.89 (s, 1H) 0.95 (s, 9H) 2.66 (dd, J = 7.32, 3.29 Hz, 1H) 4.40 (d, J = 9.88 Hz, 1H) 5.79 (s, 2H) 7.17 (t, J = 8.79 Hz, 2H) 7.28-7.35 (m, 3H) 7.46 (t, J = 7.50 Hz, 1H) 7.58 (d, J = 9.88 Hz, 1H) 7.80 (d, J = 8.79 Hz, 1H) 8.17 (d, J = 8.05 Hz, 1H) 8.36 (d, J = 4.39 Hz, 1H) | 423 |
| 81 | 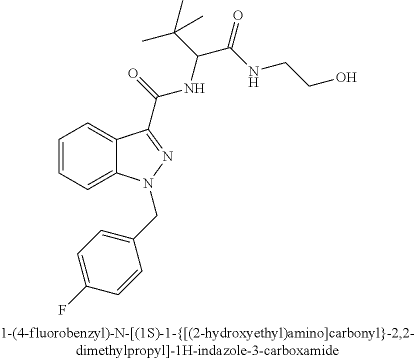 | ¹H NMR (400 MHz, DMSO-d6) δ ppm 0.97 (s, 10H) 3.13 (d, J = 5.86 Hz, 1H) 3.19 (d, J = 5.86 Hz, 1H) 3.22 (s, 1H) 3.41 (q, J = 5.98 Hz, 2H) 4.50 (d, J = 9.88 Hz, 1H) 4.68 (t, J = 5.31 Hz, 1H) 5.79 (s, 2H) 7.16 (t, J = 8.79 Hz, 2H) 7.26-7.35 (m, 3H) 7.46 (t, J = 7.69 Hz, 1H) 7.62 (d, J = 9.88 Hz, 1H) 7.80 (d, J = 8.79 Hz, 1H) 8.17 (d, J = 8.05 Hz, 1H) 8.32 (t, J = 5.67 Hz, 1H) | 427 |
| 82 | 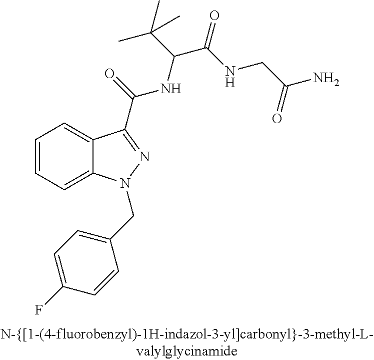 | ¹H NMR (400 MHz, DMSO-d6) δ ppm 0.86 (d, J = 8.05 Hz, 1H) 0.99 (s, 9H) 3.69 (d, J = 5.49 Hz, 2H) 4.54 (d, J = 9.52 Hz, 1H) 5.79 (s, 2H) 7.01 (br. s., 1H) 7.16 (t, J = 8.97 Hz, 2H) 7.26-7.36 (m, 4H) 7.46 (t, J = 7.69 Hz, 1H) 7.64 (d, J = 9.52 Hz, 1H) 7.79 (d, J = 8.42 Hz, 1H) 8.17 (d, J = 8.05 Hz, 1H) 8.49 (t, J = 5.67 Hz, 1H) | 440 |
| 83 | 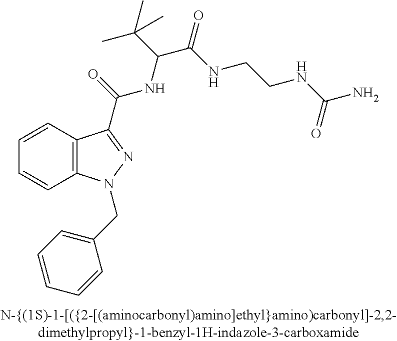 | ¹H NMR (400 MHz, DMSO-d6) δ ppm 0.97 (s, 9H) 2.99-3.10 (m, 3H) 3.11-3.21 (m, 1H) 4.45 (d, J = 9.88 Hz, 1H) 5.50 (s, 2H) 5.80 (s, 2H) 5.97 (br. s., 1H) 7.23-7.35 (m, 5H) 7.45 (t, J = 7.69 Hz, 1H) 7.62 (d, J = 9.52 Hz, 1H) 7.78 (d, J = 8.42 Hz, 1H) 8.17 (d, J = 8.05 Hz, 1H) 8.31-8.40 (m, 1H) | 451 |
| 84 | 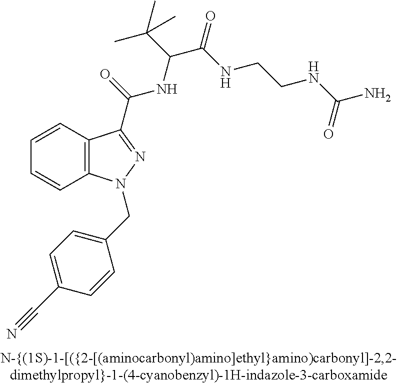 | ¹H NMR (400 MHz, DMSO-d6) δ ppm 0.97 (s, 9H) 2.99-3.10 (m, 3H) 3.11-3.20 (m, 1H) 4.45 (d, J = 9.52 Hz, 1H) 5.50 (s, 2H) 5.93 (s, 2H) 5.97 (br. s., 1H) 7.29-7.39 (m, 3H) 7.47 (t, J = 7.50 Hz, 1H) 7.60 (d J = 9.88 Hz, 1H) 7.77 (d, J = 8.42 Hz, 1H) 7.81 (d, J = 8.42 Hz, 2H) 8.19 (d, J = 8.05 Hz, 1H) 8.32-8.40 (m, 1H) | 476 |
| 85 | 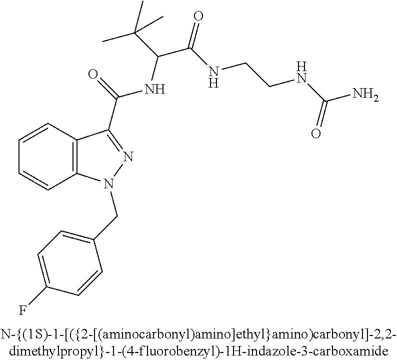 | ¹H NMR (400 MHz, DMSO-d6) δ ppm 0.91 (br. s., 1H) 0.97 (s, 9H) 2.99-3.10 (m, 3H) 3.11-3.22 (m, 1H) 4.45 (d, J = 9.88 Hz, 1H) 5.50 (s, 2H) 5.79 (s, 2H) 5.97 (br. s., 1H) 7.17 (t, J = 8.79 Hz, 2H) 7.26-7.36 (m, 3H) 7.46 (t, J = 7.50 Hz, 1H) 7.61 (d, J = 9.52 Hz, 1H) 7.80 (d, J = 8.79 Hz, 1H) 8.17 (d, J = 8.05 Hz, 1H) 8.30-8.40 (m, 1H) | 469 |
| 86 | 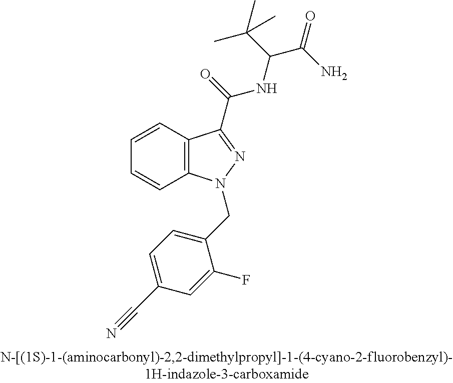 | ¹H NMR (400 MHz, DMSO-d6) δ ppm 0.96 (s, 9H) 3.31 (s, 1H) 4.44 (d, J = 9.88 Hz, 1H) 5.95 (s, 2H) 7.15 (t, J = 7.69 Hz, 1H) 7.26 (s, 1H) 7.31 (t, J = 7.50 Hz, 1H) 7.48 (t, J = 7.32 Hz, 1H) 7.55 (d, J = 9.52 Hz, 1H) 7.63 (d, J = 8.79 Hz, 1H) 7.72 (br. s., 1H) 7.78 (d, J = 8.42 Hz, 1H) 7.91 (d, J = 10.25 Hz, 1H) 8.19 (d, J = 8.05 Hz, 1H) | 408 |
| 87 | 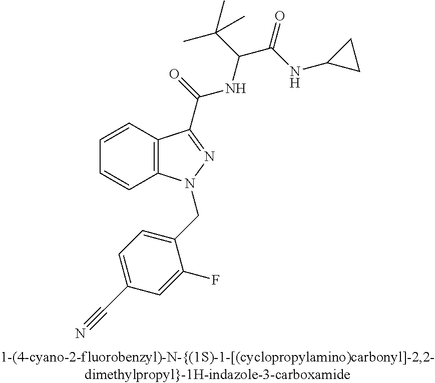 | ¹H NMR (400 MHz, DMSO-d6) δ ppm 0.33-0.41 (m, 2H) 0.41 (br. s., 1H) 0.56-0.66 (m, 2H) 0.93 (s, 10H) 2.64 (dd, J = 7.32, 3.29 Hz, 1H) 3.31 (s, 1H) 4.38 (d, J = 9.88 Hz, 1H) 5.95 (s, 2H) 7.17 (t, J = 7.69 Hz, 1H) 7.31 (t, J = 7.50 Hz, 1H) 7.51 (dd, J = 12.81, 8.79 Hz, 2H) 7.64 (d, J = 6.95 Hz, 1H) 7.78 (d, J = 8.42 Hz, 1H) 7.91 (d, J= 9.88 Hz, 1H) 8.18 (d, J = 8.42 Hz, 1H) 8.35 (d, J = 4.39 Hz, 1H) | 448 |
| 88 | 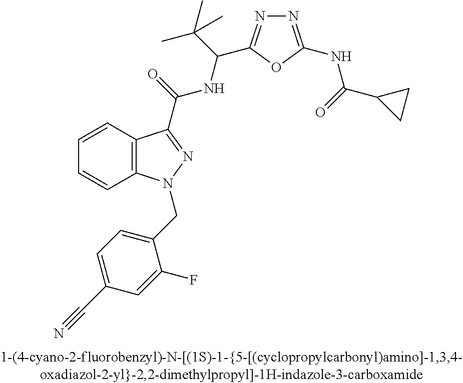 | ¹H NMR (400 MHz, DMSO-d6) δ ppm 0.81-0.91 (m, 4H) 0.94-1.01 (m, 3H) 1.04 (s, 7H) 1.86 (br. s., 1H) 3.31 (s, 1H) 5.23 (d, J = 9.52 Hz, 1H) 5.95 (s, 2H) 7.14-7.19 (m, 1H) 7.32 (t, J = 7.50 Hz, 1H) 7.49 (t, J = 7.87 Hz, 1H) 7.63 (d, J = 8.05 Hz, 1H) 7.78 (d, J = 8.42 Hz, 1H) 7.91 (d, J = 9.88 Hz, 1H) 8.11-8.20 (m, 2H) | 516 |
| 89 | 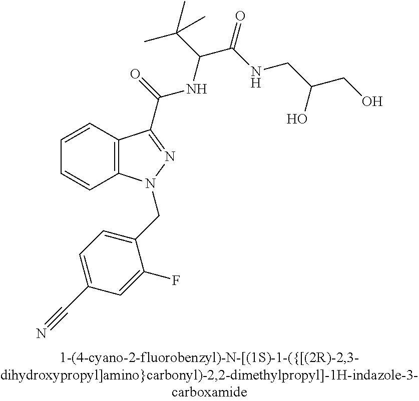 | ¹H NMR (400 MHz, DMSO-d6) δ ppm 0.95 (s, 9H) 3.01-3.11 (m, 1H) 3.12-3.23 (m, 1H) 3.28 (t, J = 5.49 Hz, 2H) 3.47 (d, J = 5.86 Hz, 1H) 4.48-4.56 (m, 2H) 4.73 (d, J = 5.12 Hz, 1H) 5.95 (s, 2H) 7.16 (t, J = 7.69 Hz, 1H) 7.31 (t, J = 7.50 Hz, 1H) 7.48 (t, J = 7.69 Hz, 1H) 7.57 (d, J = 9.88 Hz, 1H) 7.64 (d, J = 8.05 Hz, 1H) 7.78 (d, J = 8.42 Hz, 1H) 7.91 (d, J = 9.88 Hz, 1H) 8.18 (d, J = 8.42 Hz, 1H) 8.27 (t, J = 5.67 Hz, 1H) | 482 |
| 90 | 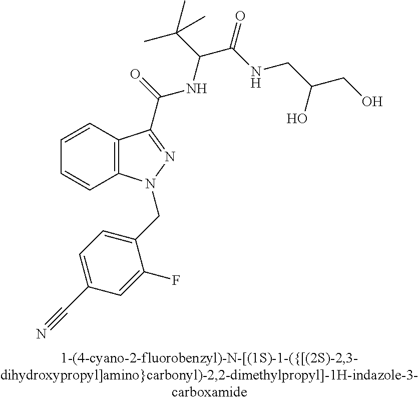 | ¹H NMR (400 MHz, DMSO-d6) δ ppm 0.95 (s, 10H) 2.95 (d, J = 6.95 Hz, 1H) 3.22-3.31 (m, 3H) 3.49 (d, J = 5.86 Hz, 1H) 4.49-4.58 (m, 2H) 4.72 (d, J = 4.76 Hz, 1H) 5.95 (s, 2H) 7.16 (t, J = 7.69 Hz, 1H) 7.31 (t, J = 7.50 Hz, 1H) 7.48 (t, J = 7.69 Hz, 1H) 7.57 (d, J = 9.88 Hz, 1H) 7.63 (d, J = 8.05 Hz, 1H) 7.78 (d, J = 8.42 Hz, 1H) 7.91 (d, J = 9.15 Hz, 1H) 8.18 (d, J = 8.05 Hz, 1H) 8.26 (t, J = 5.49 Hz, 1H) | 482 |
| 91 | 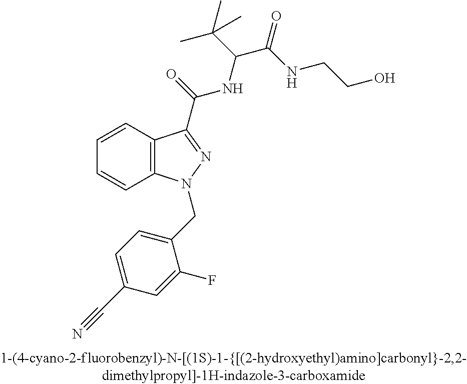 | ¹H NMR (400 MHz, DMSO-d6) δ ppm 0.94 (s, 10H) 3.11 (d, J = 5.86 Hz, 1H) 3.14-3.25 (m, 1H) 3.40 (q, J = 5.61 Hz, 2H) 4.48 (d, J = 9.52 Hz, 1H) 4.67 (t, J = 5.31 Hz, 1H) 5.95 (s, 2H) 7.15 (t, J = 7.69 Hz, 1H) 7.31 (t, J = 7.50 Hz, 1H) 7.48 (t, J = 7.69 Hz, 1H) 7.56 (d, J = 9.52 Hz, 1H) 7.64 (d, J = 8.05 Hz, 1H) 7.78 (d, J = 8.42 Hz, 1H) 7.91 (d, J = 9.88 Hz, 1H) 8.18 (d, J = 8.05 Hz, 1H) 8.30 (t, J = 5.49 Hz, 1H) | 452 |
| 92 | 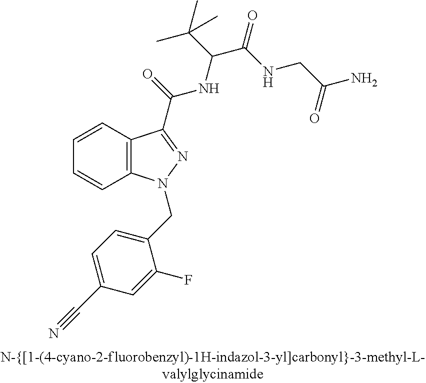 | ¹H NMR (400 MHz, DMSO-d6) δ ppm 0.84 (s, 1H) 0.97 (s, 9H) 3.31 (s, 1H) 3.67 (d, J = 5.86 Hz, 2H) 4.52 (d, J = 9.52 Hz, 1H) 5.95 (s, 2H) 7.00 (br. s., 1H) 7.15 (t, J = 7.69 Hz, 1H) 7.31 (t, J = 7.32 Hz, 2H) 7.48 (t, J = 7.50 Hz, 1H) 7.55-7.65 (m, 2H) 7.78 (d, J = 8.42 Hz, 1H) 7.91 (d, J = 9.15 Hz, 1H) 8.18 (d, J = 8.42 Hz, 1H) 8.48 (t, J = 5.67 Hz, 1H) | 465 |
| 93 | 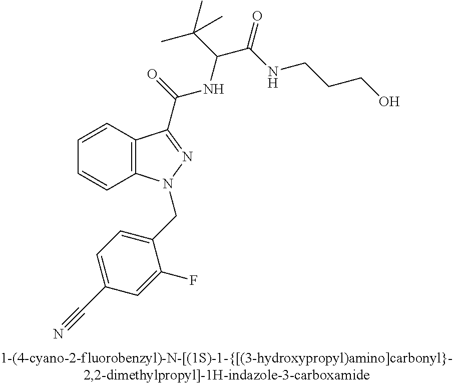 | ¹H NMR (400 MHz, DMSO-d6) δ ppm 0.94 (s, 10H) 1.55 (td, J = 6.59, 2.20 Hz, 2H) 2.99-3.09 (m, 1H) 3.15-3.25 (m, 1H) 3.33 (s, 1H) 3.39 (d, J = 5.86 Hz, 1H) 4.40-4.48 (m, 2H) 5.95 (s, 2H) 7.16 (t, J = 7.69 Hz, 1H) 7.31 (t, J = 7.50 Hz, 1H) 7.48 (t, J = 7.69 Hz, 1H) 7.55 (d, J = 9.52 Hz, 1H) 7.64 (d, J = 7.69 Hz, 1H) 7.78 (d, J = 8.42 Hz, 1H) 7.91 (d, J = 9.15 Hz, 1H) 8.18 (d, J = 8.05 Hz, 1H) 8.27 (t, J = 5.31 Hz, 1H) | 466 |
| 94 | 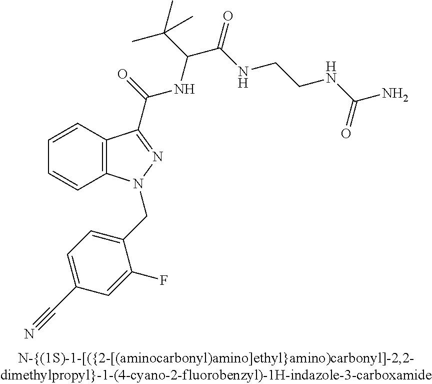 | ¹H NMR (400 MHz, DMSO-d6) δ ppm 0.94 (s, 8H) 3.00-3.07 (m, 2H) 3.14 (d, J = 6.59 Hz, 1H) 4.42 (d, J = 9.52 Hz, 1H) 5.49 (s, 2H) 5.93 (d, J = 13.18 Hz, 3H) 5.96 (br. s., 1H) 7.13-7.24 (m, 1H) 7.32 (q, J = 7.69 Hz, 1H) 7.49 (td, J = 7.50, 4.39 Hz, 1H) 7.55 (d, J = 9.88 Hz, 1H) 7.64 (td, J = 4.03, 2.93 Hz, 1H) 7.78 (d, J = 8.42 Hz, 1H) 7.83 (d, J = 8.79 Hz, 1H) 7.91 (dd, J = 7.69, 2.20 Hz, 1H) 8.10 (d, J = 8.05 Hz, 1H) 8.18 (d, J = 8.42 Hz, 1H) 8.32-8.37 (m, 1H) | 494 |
| 95 | 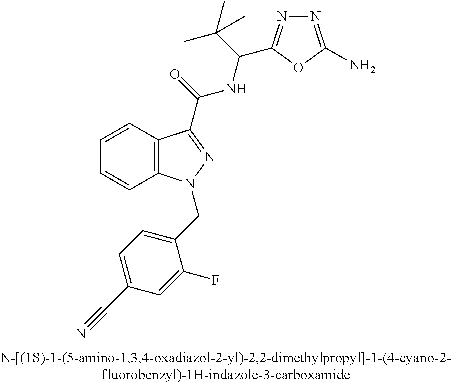 | ¹H NMR (400 MHz, DMSO-d6) ppm 0.96 (s, 9H) 1.33 (t, J = 12.81 Hz, 2H) 1.60 (br. s., 1H) 1.70 (br. s., 1H) 1.81 (br. s., 1H) 1.96 (br. s., 2H) 2.09 (br. s., 1H) 3.02-3.12 (m, 1H) 3.13-3.23 (m, 1H) 3.25-3.32 (m, 3H) 3.48 (d, J = 5.49 Hz, 1H) 4.45 (d, J = 6.95 Hz, 2H) 4.49-4.57 (m, 2H) 4.75 (d, J = 5.12 Hz, 1H) 7.27 (t, J = 7.50 Hz, 1H) 7.46 (t, J = 7.69 Hz, 1H) 7.57 (d, J = 9.52 Hz, 1H) 7.84 (d, J = 8.79 Hz, 1H) 8.15 (d, J = 8.42 Hz, 1H) 8.28 (t, J = 5.67 Hz, 1H) | 448 |
| 96 | 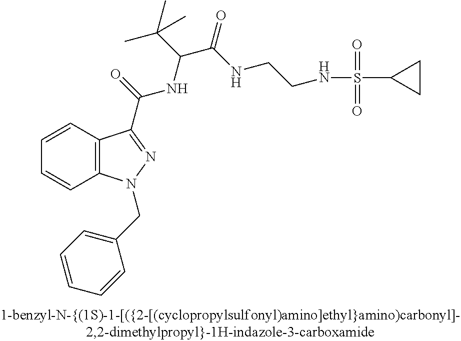 | ¹H NMR (400 MHz, DMSO-d6) δ ppm 0.82-0.94 (m, 5H) 0.97 (s, 9H) 3.03 (d, J = 5.86 Hz, 2H) 3.22 (dt, J = 13.27, 6.73 Hz, 2H) 4.45 (d, J = 9.88 Hz, 1H) 5.79 (s, 2H) 7.12 (br. s., 1H) 7.22-7.34 (m, 5H) 7.45 (t, J = 7.69 Hz, 1H) 7.61 (d, J = 9.52 Hz, 1H) 7.77 (d, J = 8.42 Hz, 1H) 8.16 (d, J = 8.05 Hz, 1H) 8.41 (t, J = 5.49 Hz, 1H) | 512 |
| 97 | 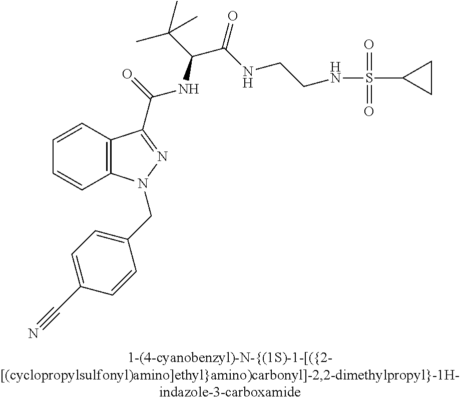 | ¹H NMR (400 MHz, DMSO-d6) δ ppm 0.82-0.93 (m, 5H) 0.96 (s, 9H) 2.51 (br. s., 1H) 3.03 (d, J = 5.86 Hz, 2H) 3.21 (dt, J = 13.45, 7.00 Hz, 2H) 4.45 (d, J = 9.52 Hz, 1H) 5.92 (s, 2H) 7.12 (t, J = 5.86 Hz, 1H) 7.27-7.38 (m, 3H) 7.47 (t, J = 7.69 Hz, 1H) 7.60 (d, J = 9.88 Hz, 1H) 7.77 (d, J = 8.42 Hz, 1H) 7.80 (d, J = 8.42 Hz, 2H) 8.18 (d, J = 8.05 Hz, 1H) 8.40 (t, J = 5.4 Hz, 1H) | 537 |
| 98 | 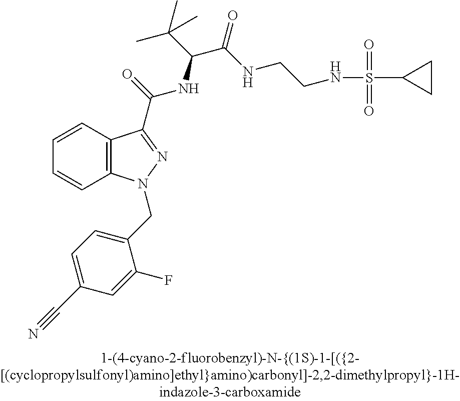 | ¹H NMR (400 MHz, DMSO-d6) δ ppm 0.82-0.93 (m, 5H) 0.95 (s, 9H) 2.51 (br. s., 1H) 3.02 (d, J = 5.86 Hz, 2H) 3.04 (br. s., 1H) 3.15-3.27 (m, 2H) 4.44 (d, J = 9.52 Hz, 1H) 5.95 (s, 2H) 7.11 (t, J = 5.86 Hz, 1H) 7.16 (t, J = 7.69 Hz, 1H) 7.31 (t, J = 7.50 Hz, 1H) 7.49 (t, J = 7.69 Hz, 1H) 7.55 (d, J = 9.52 Hz, 1H) 7.64 (d, J = 7.69 Hz, 1H) 7.79 (d, J = 8.42 Hz, 1H) 7.92 (d, J = 9.88 Hz, 1H) 8.18 (d, J = 8.05 Hz, 1H) 8.40 (t, J = 5.49 Hz, 1H) | 555 |
| 99 | 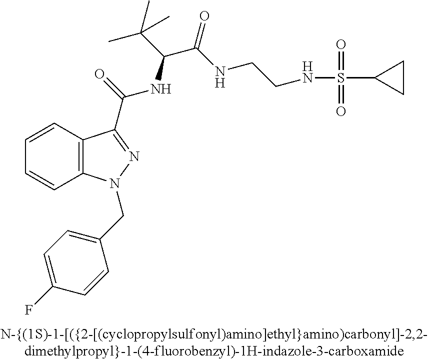 | ¹H NMR (400 MHz, DMSO-d6) δ ppm 0.82-0.94 (m, 5H) 0.97 (s, 9H) 2.51 (br. s., 1H) 3.03 (d, J = 6.22 Hz, 2H) 3.05 (br. s., 1H) 3.22 (dt, J = 13.36, 6.86 Hz, 2H) 4.45 (d, J = 9.52 Hz, 1H) 5.78 (s, 2H) 7.09-7.19 (m, 3H) 7.25-7.35 (m, 3H) 7.45 (t, J = 7.69 Hz, 1H) 7.60 (d, J = 9.88 Hz, 1H) 7.79 (d, J = 8.42 Hz, 1H) 8.16 (d, J = 8.05 Hz, 1H) 8.40 (t, J = 5.49 Hz, 1H) | 530 |
| 100 | 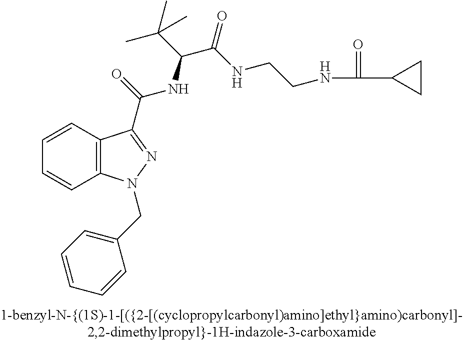 | ¹H NMR (400 MHz, DMSO-d6) δ ppm 0.56-0.66 (m, 4H) 0.97 (s, 9H) 1.43-1.50 (m, 1H) 3.05 (s, 1H) 3.11-3.22 (m, 3H) 3.32 (s, 1H) 4.44 (d, J = 9.52 Hz, 1H) 5.71 (s, 1H) 5.76-5.85 (m, 1H) 5.89-5.96 (m, 1H) 7.22-7.33 (m, 8H) 7.70 (d, J = 8.42 Hz, 1H) 7.77 (t, J = 4.21 Hz, 1H) 8.06-8.15 (m, 1H) 8.16-8.26 (m, 2H) | 476 |
| 101 | 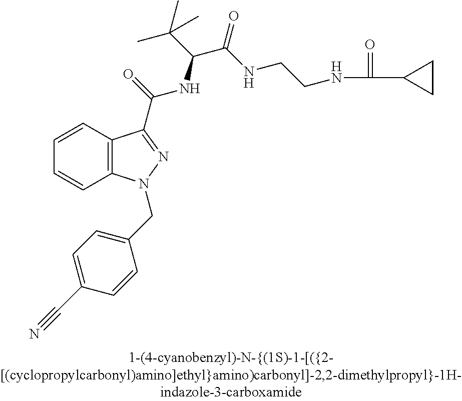 | ¹H NMR (400 MHz, DMSO-d6) δ ppm 0.55-0.64 (m, 4H) 0.95 (s, 9H) 1.42-1.49 (m, 1H) 3.07-3.17 (m, 3H) 3.20 (d, J = 6.95 Hz, 1H) 4.44 (d, J = 9.88 Hz, 1H) 5.92 (s, 2H) 7.28-7.37 (m, 3H) 7.46 (t, J = 7.69 Hz, 1H) 7.60 (d, J = 9.52 Hz, 1H) 7.76 (d, J = 8.79 Hz, 1H) 7.80 (d, J = 8.05 Hz, 2H) 8.06 (t, J = 4.76 Hz, 1H) 8.18 (d, J = 8.05 Hz, 1H) 8.32-8.38 (m, 1H) | 501 |
| 102 | 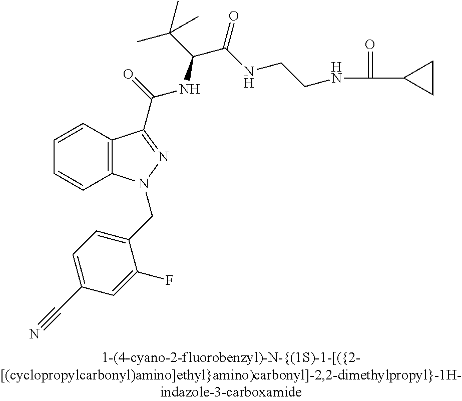 | ¹H NMR (400 MHz, DMSO-d6) δ ppm 0.96 (s, 11H) 1.27 (d, J = 7.32 Hz, 4H) 1.33 (br. s., 1H) 1.36 (d, J = 8.42 Hz, 1H) 1.51 (br. s., 1H) 1.95 (d, J = 9.52 Hz, 2H) 4.27 (t, J = 7.32 Hz, 1H) 4.45 (d, J = 7.32 Hz, 2H) 4.60 (d, J = 9.88 Hz, 1H) 7.27 (t, J = 7.50 Hz, 1H) 7.46 (t, J = 7.69 Hz, 1H) 7.55 (d, J = 9.88 Hz, 1H) 7.84 (d, J = 8.42 Hz, 1H) 8.16 (d, J = 8.05 Hz, 1H) 8.68 (d, J = 7.32 Hz, 1H) | 519 |
| 103 | 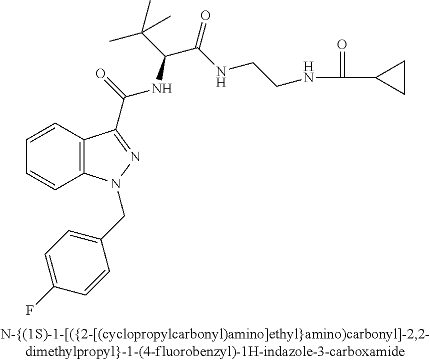 | ¹H NMR (400 MHz, DMSO-d6) δ ppm 0.55-0.64 (m, 4H) 0.96 (s, 9H) 1.42-1.49 (m, 1H) 3.08-3.17 (m, 3H) 3.21 (br. s., 1H) 4.44 (d, J = 9.52 Hz, 1H) 5.78 (s, 2H) 7.15 (t, J = 8.79 Hz, 2H) 7.26-7.33 (m, 3H) 7.45 (t, J = 7.69 Hz, 1H) 7.60 (d, J = 9.52 Hz, 1H) 7.79 (d, J = 8.42 Hz, 1H) 8.03-8.09 (m, 1H) 8.16 (d, J = 8.05 Hz, 1H) 8.32-8.38 (m, 1H) | 494 |
| 104 | 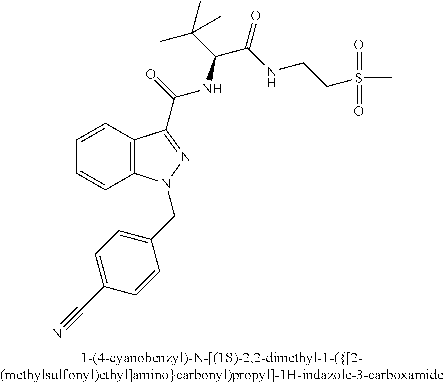 | ¹H NMR (400 MHz, DMSO-d6) δ ppm 0.91 (s, 1H) 0.96 (s, 9H) 2.99 (s, 3H) 3.21-3.31 (m, 2H) 3.43-3.54 (m, 2H) 4.46 (d, J = 9.52 Hz, 1H) 5.92 (s, 2H) 7.28-7.37 (m, 3H) 7.46 (t, J = 7.69 Hz, 1H) 7.60 (d, J = 9.88 Hz, 1H) 7.76 (d, J = 8.79 Hz, 1H) 7.80 (d, J = 8.05 Hz, 2H) 8.17 (d, J = 8.05 Hz, 1H) 8.60 (t, J = 5.49 Hz, 1H) | 496 |
| 105 | 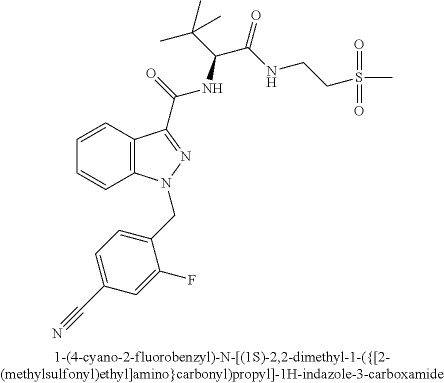 | ¹H NMR (400 MHz, DMSO-d6) δ ppm 0.91 (s, 1H) 0.95 (s, 9H) 2.99 (s, 3H) 3.26 (q, J = 6.95 Hz, 2H) 3.48 (td, J = 12.54, 6.04 Hz, 2H) 4.45 (d, J = 9.52 Hz, 1H) 5.95 (s, 2H) 7.17 (t, J = 7.69 Hz, 1H) 7.31 (t, J = 7.50 Hz, 1H) 7.49 (t, J = 7.87 Hz, 1H) 7.55 (d, J = 9.52 Hz, 1H) 7.64 (d, J = 7.69 Hz, 1H) 7.79 (d, J = 8.42 Hz, 1H) 7.91 (d, J = 10.25 Hz, 1H) 8.17 (d, J = 8.42 Hz, 1H) 8.59 (t, J = 5.49 Hz, 1H) | 514 |
| 106 | 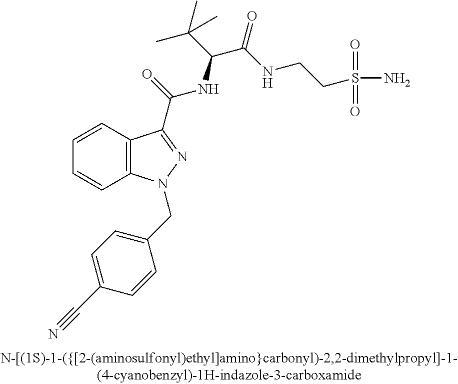 | ¹H NMR (400 MHz, DMSO-d6) δ ppm 0.96 (s, 9H) 3.07-3.17 (m, 2H) 3.41 (d, J = 5.86 Hz, 1H) 3.50 (d, J = 2.93 Hz, 1H) 4.44 (d, J = 9.52 Hz, 1H) 5.92 (s, 2H) 6.91 (s, 2H) 7.28-7.37 (m, 3H) 7.47 (t, J = 7.69 Hz, 1H) 7.59 (d, J = 9.52 Hz, 1H) 7.76 (d, J = 8.42 Hz, 1H) 7.80 (d, J = 8.05 Hz, 2H) 8.17 (d, J = 8.05 Hz, 1H) 8.51 (t, J = 5.49 Hz, 1H) | 497 |
| 107 | 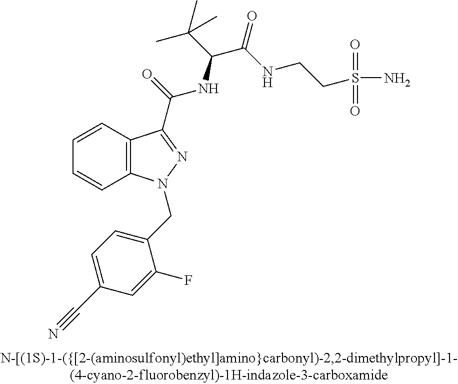 | ¹H NMR (400 MHz, DMSO-d6) δ ppm 0.95 (s, 9H) 3.11 (td, J = 9.06, 6.04 Hz, 2H) 3.40 (d, J = 5.86 Hz, 1H) 3.49 (dd, J = 8.42, 5.49 Hz, 1H) 4.42 (d, J = 9.52 Hz, 1H) 5.95 (s, 2H) 6.91 (s, 2H) 7.17 (t, J = 7.69 Hz, 1H) 7.31 (t, J = 7.50 Hz, 1H) 7.49 (t, J = 7.87 Hz, 1H) 7.55 (d, J = 9.52 Hz, 1H) 7.64 (d, J = 8.05 Hz, 1H) 7.78 (d, J = 8.42 Hz, 1H) 7.91 (d, J = 9.88 Hz, 1H) 8.17 (d, J = 8.42 Hz, 1H) 8.51 (t, J = 5.49 Hz, 1H) | 515 |
| 108 | 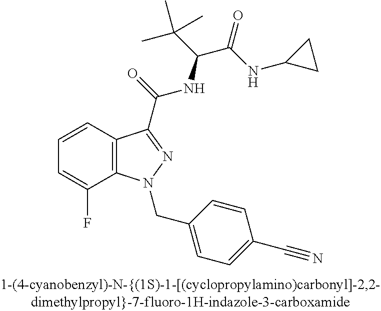 | ¹H NMR (400 MHz, DMSO-d6) δ ppm 8.37 (1H, d, J = 4.0 Hz), 8.01 (1H, d, J = 7.5 Hz), 7.82 (1H, d, J = 8.2 Hz), 7.62 (1H, d, J = 9.7 Hz), 7.24-7.35 (4H, m), 5.95 (2H, s), 4.41 (1H, d, J = 9.7 Hz), 2.62-2.71 (1H, m), 0.95 (9H, s), 0.59-0.67 (2H, m), 0.35-0.46 (2H, m) | 448 |
| 109 | 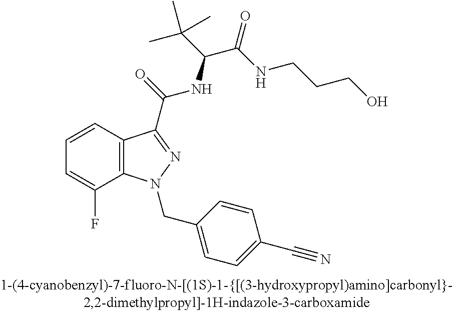 | ¹H NMR (400 MHz, DMSO-d6) δ ppm 8.29 (1H, t, J = 5.4 Hz), 7.99-8.05 (1H, m), 7.81 (1H, d, J = 8.2 Hz), 7.65 (1H, d, J = 9.5 Hz), 7.24-7.36 (4H, m), 5.94 (2H, s), 4.42-4.53 (1H, m), 3.38-3.44 (2H, m), 3.16-3.27 (1H, m), 3.01-3.12 (1H, m), 1.51-1.62 (2H, m), 0.97 (9H, s) | 466 |
| 110 | 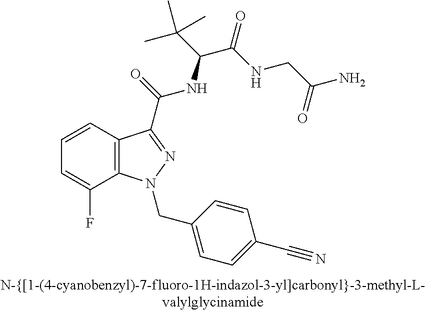 | ¹H NMR (400 MHz, DMSO-d6) δ ppm 8.50 (1H, t, J = 5.7 Hz), 7.98-8.08 (1H, m), 7.81 (1H, d, J = 8.2 Hz), 7.69 (1H, d, J = 9.3 Hz), 7.22-7.39 (3H, m), 7.02 (1H, br. s.), 5.94 (2H, s), 4.55 (1H, d, J = 9.3 Hz), 3.62-3.78 (2H, m), 0.99 (9H, s) | 465 |
| 111 | 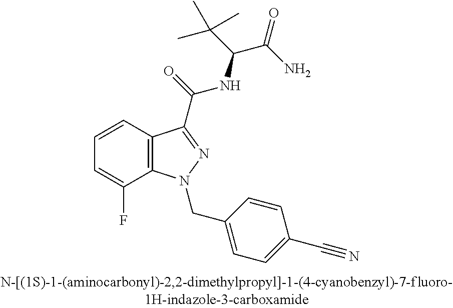 | ¹H NMR (400 MHz, DMSO-d6) δ ppm 7.71-7.85 (2H, m), 7.65 (1H, d, J = 9.7 Hz), 7.22-7.39 (4H, m), 5.94 (2H, s), 4.47 (1H, d, J = 9.7 Hz), 0.99 (9H, s) | 408 |
| 112 | 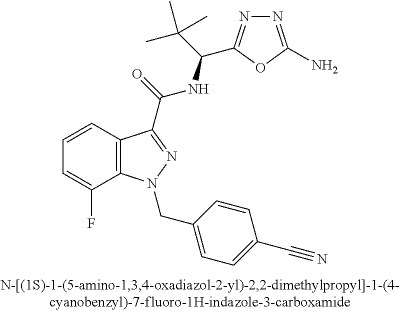 | ¹H NMR (400 MHz, DMSO-d6) δ ppm 8.22 (d, J = 9.52 Hz, 1H) 7.97 (d, J = 7.32 Hz, 1H) 7.81 (d, J = 8.05 Hz, 2H) 7.23-7.40 (m, 4H) 7.04 (s, 2H) 5.92 (s, 2H) 5.12 (d, J = 9.52 Hz, 1H) 1.04 (s, 9H). | 448 |
| 113 | 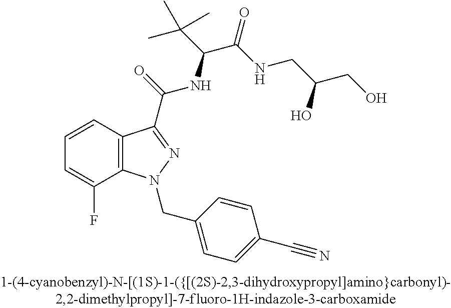 | ¹H NMR (400 MHz, DMSO-d6) δ ppm 8.27 (t, J = 5.49 Hz, 1H) 8.01 (d, J = 7.69 Hz, 1H) 7.80 (d, J = 8.05 Hz, 2H) 7.67 (d, J = 9.88 Hz, 1H) 7.22-7.40 (m, 4H) 5.93 (s, 2H) 4.72 (d, J = 5.12 Hz, 1H) 4.51-4.61 (m, 2H) 3.44-3.56 (m, 1H) 3.28 (t, J = 4.94 Hz, 2H) 2.89-3.02 (m, 1H) 0.97 (s, 9H) | 482 |
| 114 | 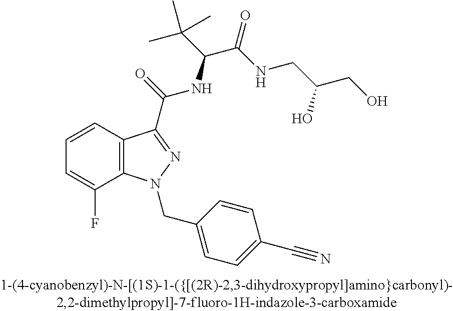 | ¹H NMR (400 MHz, DMSO-d6) δ ppm 8.28 (t, J = 5.49 Hz, 1H) 8.01 (d, J = 7.32 Hz, 1H) 7.81 (d, J = 8.05 Hz, 2H) 7.66 (d, J = 9.52 Hz, 1H) 7.21-7.35 (m, 4H) 5.93 (s, 2H) 4.74 (d, J = 5.12 Hz, 1H) 4.49-4.60 (m, 2H) 3.44-3.54 (m, 1H) 3.29 (t, J = 5.67 Hz, 2H) 3.13-3.23 (m, 1H) 3.01-3.11 (m, 1H) 0.97 (s, 9H). | 482 |
| 115 | 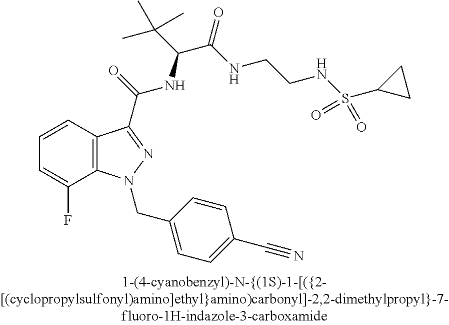 | ¹H NMR (400 MHz, DMSO-d6) δ ppm 8.41 (t, J = 5.49 Hz, 1H) 8.01 (d, J = 7.69 Hz, 1H) 7.81 (d, J = 8.05 Hz, 2H) 7.65 (d, J = 9.52 Hz, 1H) 7.21-7.37 (m, 4H) 7.12 (t, J = 5.86 Hz, 1H) 5.93 (s, 2H) 4.46 (d, J = 9.52 Hz, 1H) 3.14-3.29 (m, 2H) 2.95-3.09 (m, 2H) 0.76-1.12 (m, 13H). | 555 |
| 116 | 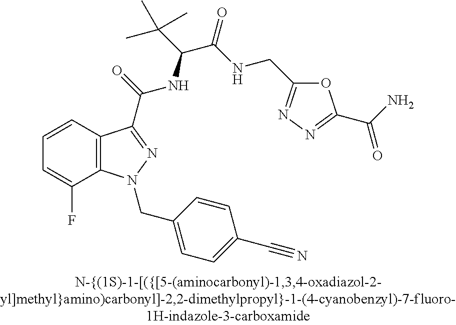 | ¹H NMR (400 MHz, DMSO-d6) δ ppm 9.16 (t, J = 5.49 Hz, 1H) 8.59 (s, 1H) 8.19 (s, 1H) 8.00 (d, J = 7.32 Hz, 1H) 7.80 (d, J = 8.42 Hz, 2H) 7.67 (d, J = 9.88 Hz, 1H) 7.22-7.35 (m, 4H) 5.93 (s, 2H) 4.53-4.72 (m, 3H) 0.98 (s, 9H). | 533 |
| 117 | 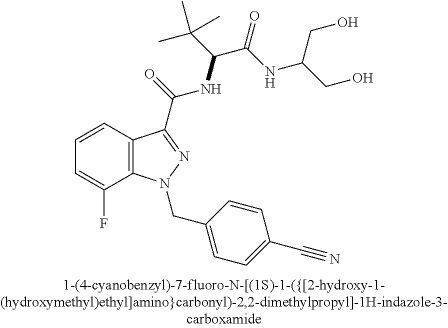 | ¹H NMR (400 MHz, DMSO-d6) δ ppm 8.04 (dd, J = 16.47, 7.69 Hz, 2H) 7.80 (d, J = 8.42 Hz, 2H) 7.65 (d, J = 9.52 Hz, 1H) 7.23-7.35 (m, 4H) 5.93 (s, 2H) 4.54-4.68 (m, 3H) 3.71-3.81 (m, 1H) 3.37-3.49 (m, 3H) 0.96 (s, 9H). | 482 |
| 118 | 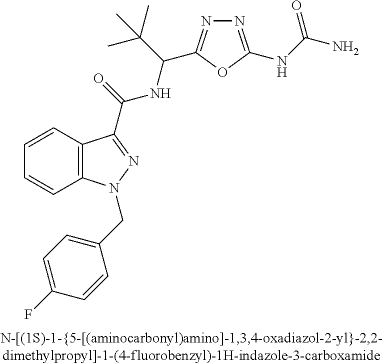 | ¹H NMR (400 MHz, DMSO-d6) δ ppm 1.01-1.10 (br. s., 9H) 5.21 (d, J = 9.52 Hz, 1H) 5.77 (s, 2H) 7.15 (t, J = 8.79 Hz, 3H) 7.26-7.37 (m, 3H) 7.45 (t, J = 7.69 Hz, 1H) 7.78 (d, J = 8.42 Hz, 1H) 8.12 (d, J = 8.05 Hz, 1H) 8.22 (d, J = 9.15 Hz, 1H) 10.63 (s, 1H) | 466 |
| 119 | 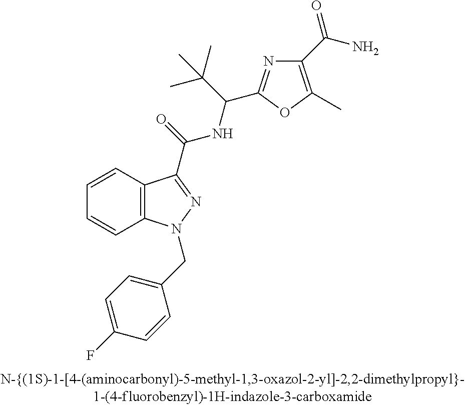 | ¹H NMR (400 MHz, DMSO-d6) δ ppm 1.03 (s, 9H) 5.14 (d, J = 9.52 Hz, 1H) 5.78 (s, 3H) 7.15 (t, J = 8.97 Hz, 2H) 7.26-7.37 (m, 3H) 7.39-7.48 (m, 3H) 7.78 (d, J = 8.42 Hz, 1H) 7.99 (d, J = 9.52 Hz, 1H) 8.12 (d, J = 8.05 Hz, 1H) | 464 |
| 120 | 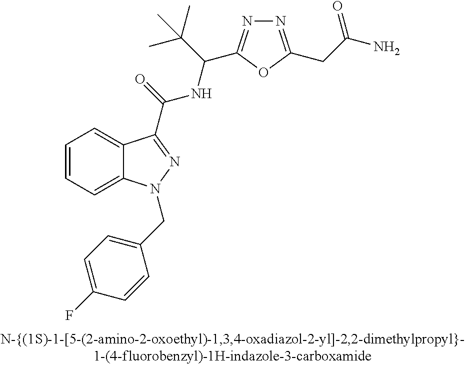 | ¹H NMR (400 MHz, DMSO-d6) δ ppm 1.04 (s, 9H) 3.83 (s, 2H) 5.31 (d, J = 9.52 Hz, 1H) 5.78 (s, 2H) 7.16 (t, J = 8.79 Hz, 2H) 7.22-7.32 (m, 2H) 7.35 (dd, J = 8.42, 5.49 Hz, 2H) 7.45 (t, J = 7.69 Hz, 1H) 7.71 (br. s., 1H) 7.79 (d, J = 8.42 Hz, 1H) 8.12 (d, J = 8.42 Hz, 1H) 8.20 (d, J = 9.52 Hz, 1H) | 465 |
| 121 | 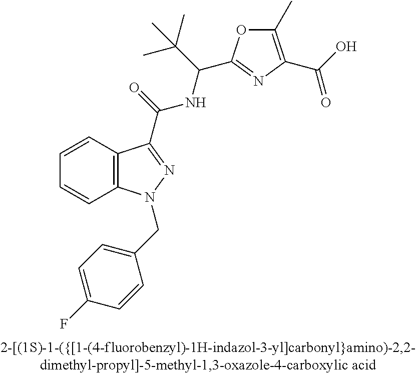 | ¹H NMR (400 MHz, DMSO-d6) δ ppm 1.02 (s, 9H) 2.55 (s, 3H) 5.13 (d, J = 9.52 Hz, 1H) 5.79 (s, 2H) 7.15 (t, J = 8.79 Hz, 2H) 7.28 (t, J = 7.50 Hz, 1H) 7.35 (dd, J = 8.42, 5.49 Hz, 2H) 7.45 (t, J = 7.69 Hz, 1H) 7.79 (d, J = 8.42 Hz, 1H) 7.99 (d, J = 9.52 Hz, 1H) 8.12 (d, J = 8.05 Hz, 1H) | 465 |
| 122 | 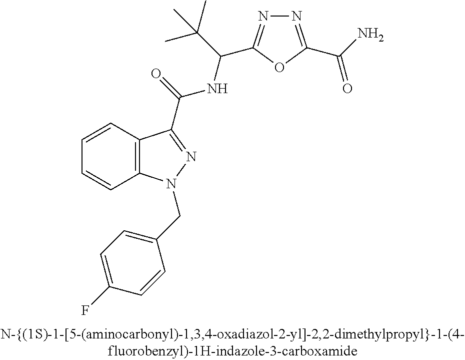 | ¹H NMR (400 MHz, DMSO-d6) δ ppm 1.08 (s, 9H) 5.38 (d, J = 9.15 Hz, 1H) 5.79 (s, 2H) 7.15 (t, J = 8.79 Hz, 2H) 7.29 (t, J = 7.50 Hz, 1H) 7.34 (dd, J = 8.42, 5.49 Hz, 2H) 7.45 (t, J = 7.69 Hz, 1H) 7.78 (d, J = 8.79 Hz, 1H) 8.11 (d, J = 8.42 Hz, 1H) 8.23 (s, 1H) 8.41 (d, J = 9.15 Hz, 1H) 8.62 (s, 1H) | 451 |
| 123 | 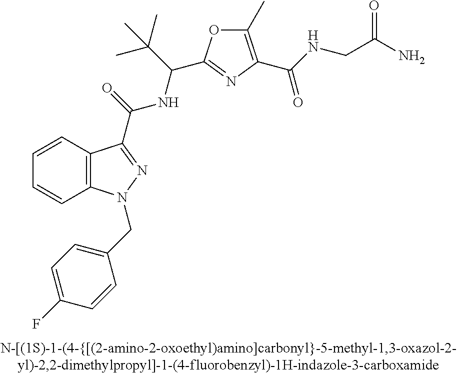 | ¹H NMR (400 MHz, DMSO-d6) δ ppm 1.04 (s, 9H) 2.55 (s, 3H) 3.77 (d, J = 5.49 Hz, 2H) 5.16 (d, J = 9.15 Hz, 1H) 5.78 (s, 2H) 7.07 (s, 1H) 7.15 (t, J = 8.79 Hz, 2H) 7.28 (t, J = 7.50 Hz, 1H) 7.35 (dd, J = 8.42, 5.49 Hz, 2H) 7.39 (s, 1H) 7.45 (t. J = 7.69 Hz, 1H) 7.78 (d, J = 8.79 Hz, 1H) 7.98-8.05 (m, 2H) 8.12 (d, J = 8.05 Hz, 1H) | 521 |
| 124 | 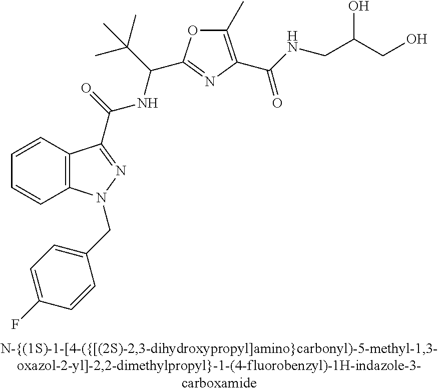 | ¹H NMR (400 MHz, DMSO-d6) δ ppm 1.04 (s, 9H) 2.55 (s, 3H) 3.09-3.18 (m, 1H) 3.24-3.30 (m, 1H) 3.36-3.43 (m, 1H) 3.52-3.60 (m, 1H) 4.61 (t, J = 5.31 Hz, 1H) 4.87 (d, J = 4.76 Hz, 1H) 5.15 (d, J = 9.52 Hz, 1H) 5.78 (s, 2H) 7.15 (t, J = 8.97 Hz, 2H) 7.28 (t, J = 7.69 Hz, 1H) 7.34 (dd, J = 8.42, 5.49 Hz, 2H) 7.45 (t, J = 7.69 Hz, 1H) 7.75-7.84 (m, 2H) 8.03 (d, J = 9.52 Hz, 1H) 8.11 (d, J = 8.05 Hz, 1H) | 538 |
| 125 | 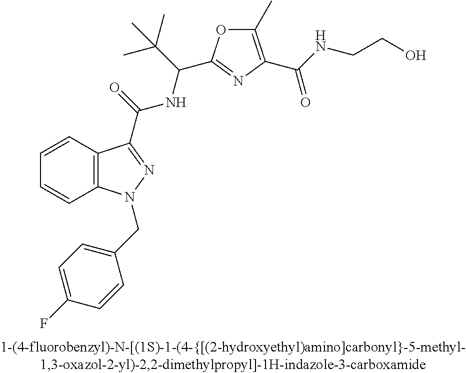 | ¹H NMR (400 MHz, DMSO-d6) δ ppm 1.04 (s, 9H) 2.55 (s, 3H) 3.24-3.30 (m, 2H) 3.45 (q, J = 5.86 Hz, 2H) 4.74 (t, J = 5.49 Hz, 1H) 5.15 (d, J = 9.52 Hz, 1H) 5.78 (s, 2H) 7.15 (t, J = 8.97 Hz, 2H) 7.28 (t, J = 7.69 Hz, 1H) 7.34 (dd, J = 8.60, 5.67 Hz, 2H) 7.45 (t, J = 7.87 Hz, 1H) 7.78 (d, J = 8.79 Hz, 1H) 7.90 (t, J = 5.67 Hz, 1H) 8.00 (d, J = 9.52 Hz, 1H) 8.11 (d, J = 8.05 Hz, 1H) | 508 |
| 126 | 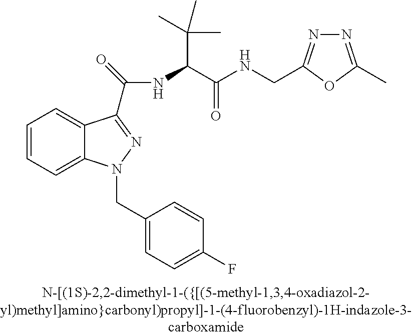 | ¹H NMR (400 MHz, DMSO-d6) δ ppm 0.98 (s, 9H) 2.43 (s, 3H) 4.41-4.62 (m, 3H) 5.77 (s, 2H) 7.15 (t, J = 8.79 Hz, 2H) 7.25-7.36 (m, 3H) 7.46 (t, 1H) 7.62 (d, J = 9.52 Hz, 1H) 7.79 (d, J = 8.79 Hz, 1H) 8.16 (d, J = 8.05 Hz, 1H) 9.06 (t, J = 5.49 Hz, 1H) | 479 |
| 127 | 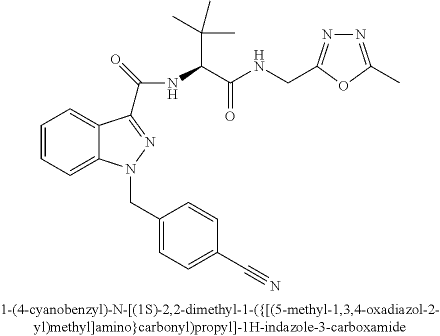 | ¹H NMR (400 MHz, DMSO-d6) δ ppm 0.97 (s, 9H) 2.43 (s, 3H) 4.40-4.61 (m, 3H) 5.92 (s, 2H) 7.31 (t, 1H) 7.35 (d, J = 8.05 Hz, 2H) 7.47 (t, 1H) 7.61 (d, J = 9.52 Hz, 1H) 7.77 (d, J = 8.79 Hz, 1H) 7.80 (d, J = 8.79 Hz, 2H) 8.18 (d, J = 8.05 Hz, 1H) 9.06 (t, J = 5.86 Hz, 1H) | 486 |
| 128 | 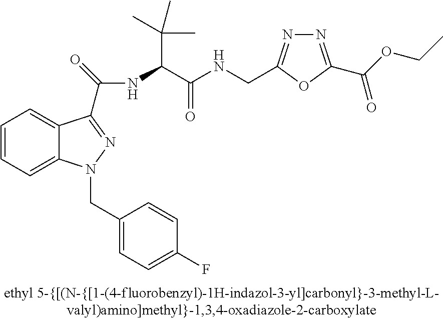 | ¹H NMR (400 MHz, DMSO-d6) δ ppm 0.99 (s, 9H) 1.28 (t, J = 7.32 Hz, 3H) 4.37 (q, J = 7.32 Hz, 2H) 4.53-4.75 (m, 3H) 5.77 (s, 2H) 7.15 (t, J = 8.79 Hz, 2H) 7.25-7.35 (m, 3H) 7.46 (t, J = 8.05 Hz, 1H) 7.61 (d, J = 9.52 Hz, 1H) 7.79 (d, J = 8.79 Hz, 1H) 8.16 (d, J = 8.05 Hz, 1H) 9.20 (t, J = 5.49 Hz, 1H) | 537 |
| 129 | 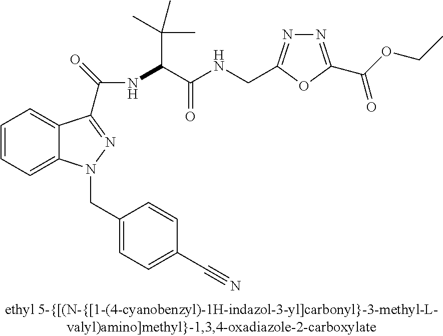 | ¹H NMR (400 MHz, DMSO-d6) δ ppm 0.89-1.08 (m, 9H) 1.28 (t, J = 7.32 Hz, 3H) 4.37 (q, J = 7.32 Hz, 2H) 4.49-4.77 (m, 3H) 5.91 (s, 2H) 7.23-7.41 (m, 3H) 7.47 (t, J = 7.69 Hz, 1H) 7.61 (d, J = 9.52 Hz, 1H) 7.72-7.87 (m, 3H) 8.18 (d, J = 8.05 Hz, 1H) 9.20 (t, J = 5.13 Hz, 1H) | 545 |
| 130 | 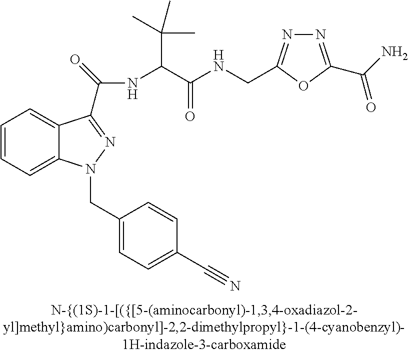 | ¹H NMR (400 MHz, DMSO-d6) δ ppm 0.98 (s, 9H) 4.50-4.72 (m, 3H) 5.85-5.96 (m, 2H) 7.25-7.40 (m, 3H) 7.47 (t, J = 7.69 Hz, 1H) 7.61 (d, J = 9.52 Hz, 1H) 7.71-7.84 (m, 2H) 7.91 (d, J = 8.05 Hz, 1H) 8.17 (s, 1H) 8.19 (br. s., 1H) 8.58 (s, 1H) 9.16 (t, J = 5.49 Hz, 1H) | 515 |
| 131 | 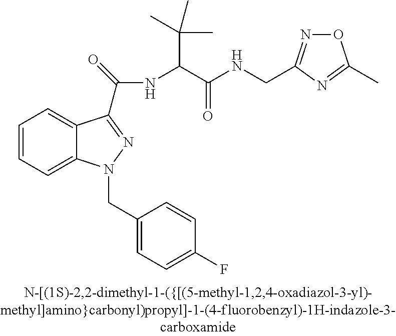 | ¹H NMR (400 MHz, DMSO-d6) δ ppm 0.98 (s, 9H) 2.54 (s, 3H) 4.25-4.64 (m, 3H) 5.78 (s, 2H) 7.15 (t, J = 8.79 Hz, 2H) 7.22-7.37 (m, 3H) 7.46 (t, J = 7.69 Hz, 1H) 7.62 (d, J = 9.52 Hz, 1H) 7.79 (d, J = 8.79 Hz, 1H) 8.16 (d, J = 8.05 Hz, 1H) 8.97 (t, J = 5.86 Hz, 1H) | 479 |
| 132 | 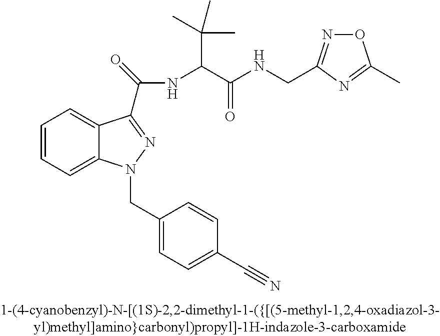 | ¹H NMR (400 MHz, DMSO-d6) δ ppm 0.97 (s, 9H) 2.54 (s, 3H) 4.26-4.62 (m, 3H) 5.92 (s, 2H) 7.22-7.41 (m, 3H) 7.47 (t, J = 7.69 Hz, 1H) 7.61 (d, J = 9.52 Hz, 1H) 7.73-7.85 (m, 3H) 8.18 (d, J = 8.05 Hz, 1H) 8.96 (t, J = 5.49 Hz, 1H) | 486 |
| 133 | 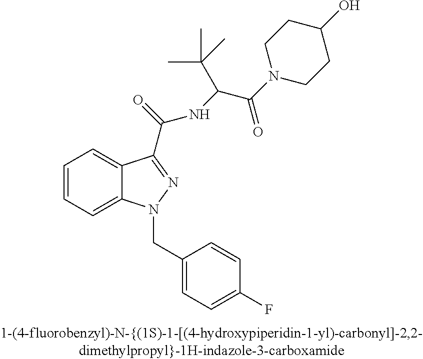 | ¹H NMR (400 MHz, DMSO-d6) δ ppm 0.98 (d, J = 5.86 Hz, 9H) 1.63-1.86 (m, 3H) 2.99 (t, J = 10.25 Hz, 1H) 3.15 (t, J = 9.52 Hz, 1H) 3.69 (d, J = 3.66 Hz, 1H) 3.81-4.08 (m, 3H) 4.71-4.84 (m, 1H) 5.08 (d, J = 9.52 Hz, 1H) 5.77 (br. s., 2H) 7.16 (t, J = 8.42 Hz, 2H) 7.24-7.37 (m, 3H) 7.45 (t, J = 7.69 Hz, 1H) 7.62 (t, J = 9.52 Hz, 1H) 7.79 (d, J = 8.79 Hz, 1H) 8.16 (dd, J = 8.05, 4.39 Hz, 1H) | 467 |
| 134 | 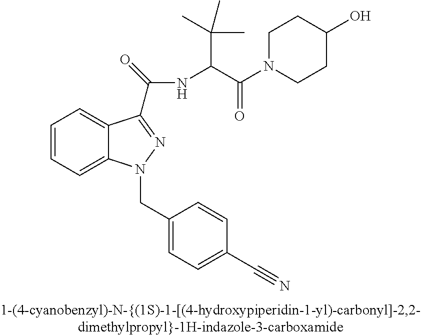 | ¹H NMR (400 MHz, DMSO-d6) δ ppm 0.98 (d, J = 5.86 Hz, 9H) 1.11-1.50 (m, 2H) 1.59-1.88 (m, 2H) 2.89-3.06 (m, 1H) 3.06-3.20 (m, 1H) 3.69 (br. s., 1H) 3.78-4.09 (m, 2H) 4.75 (br. s., 1H) 5.08 (d, J = 9.52 Hz, 1H) 5.92 (br. s., 2H) 7.23-7.42 (m, 3H) 7.47 (t, J = 7.69 Hz, 1H) 7.62 (t, J = 9.88 Hz, 1H) 7.78 (dd, J = 13.54, 8.42 Hz, 3H) 8.18 (dd, J = 8.05, 3.66 Hz, 1H) | 474 |
| 135 | 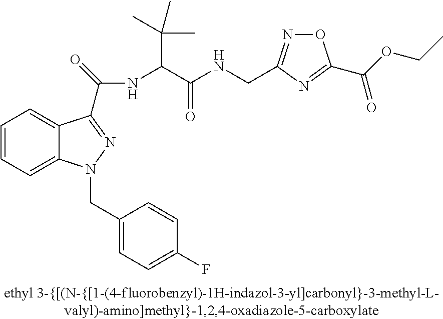 | ¹H NMR (400 MHz, DMSO-d6) δ ppm 0.98 (s, 9H) 1.29 (t, J = 6.96 Hz, 3H) 4.38 (q, J = 7.08 Hz, 2H) 4.45-4.66 (m, 3H) 5.77 (s, 2H) 7.15 (t, J = 8.79 Hz, 2H) 7.23-7.40 (m, 3H) 7.45 (t, J = 7.69 Hz, 1H) 7.62 (d, J = 9.52 Hz, 1H) 7.79 (d, J = 8.79 Hz, 1H) 8.16 (d, J = 8.05 Hz, 1H) 9.11 (t, J = 5.49 Hz, 1H) | 537 |
| 136 | 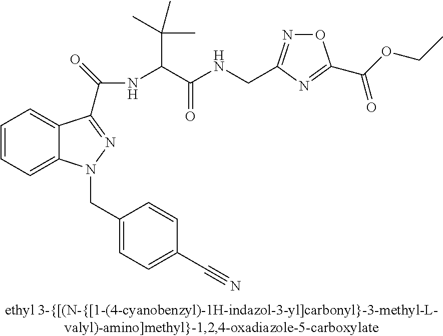 | ¹H NMR (400 MHz, DMSO-d6) δ ppm 0.98 (s, 9H) 1.29 (t, J = 6.96 Hz, 3H) 4.38 (q, J = 6.83 Hz, 2H) 4.44-4.64 (m, 3H) 5.91 (s, 2H) 7.22-7.41 (m, 3H) 7.47 (t, J = 7.69 Hz, 1H) 7.61 (d, J = 9.52 Hz, 1H) 7.70-7.85 (m, 3H) 8.18 (d, J = 8.05 Hz, 1H) 9.10 (t, J = 5.86 Hz, 1H) | 544 |
| 137 | 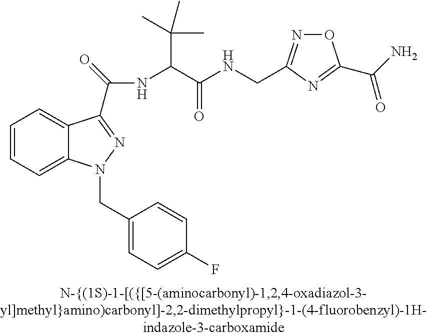 | ¹H NMR (400 MHz, DMSO-d6) δ ppm 0.99 (s, 9H) 4.33-4.66 (m, 3H) 5.64-5.88 (m, 2H) 7.16 (t, J = 8.79 Hz, 2H) 7.24-7.39 (m, 3H) 7.46 (t, J = 7.69 Hz, 1H) 7.62 (d, J = 9.52 Hz, 1H) 7.79 (d, J = 8.79 Hz, 1H) 8.16 (d, J = 8.05 Hz, 1H) 8.39 (br. s., 1H) 8.69 (br. s., 1H) 9.07 (t, J = 5.49 Hz, 1H) | 508 |
| 138 | 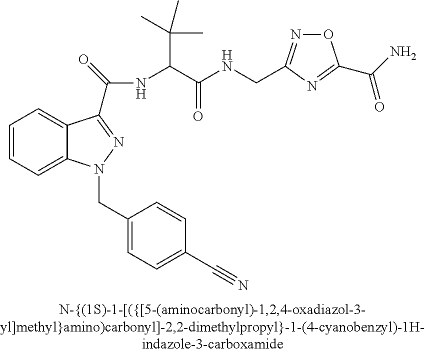 | ¹H NMR (400 MHz, DMSO-d6) δ ppm 0.98 (s, 9H) 4.39-4.66 (m, 3H) 5.85-6.01 (m, 2H) 7.25-7.41 (m, 3H) 7.47 (t, J = 7.69 Hz, 1H) 7.62 (d, J = 10.25 Hz, 1H) 7.70-7.87 (m, 3H) 8.18 (d, J = 8.05 Hz, 1H) 8.39 (br. s., 1H) 8.68 (br. s., 1H) 9.06 (t, J = 5.49 Hz, 1H) | 515 |
| 139 | 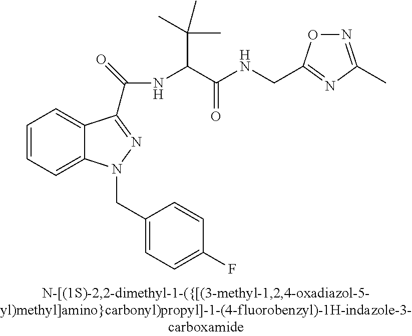 | ¹H NMR (400 MHz, DMSO-d6) δ ppm 0.99 (s, 9H) 2.28 (s, 3H) 4.44-4.70 (m, 3H) 5.77 (s, 2H) 7.15 (t, J = 9.15 Hz, 2H) 7.23-7.39 (m, 3H) 7.46 (t, J = 7.69 Hz, 1H) 7.62 (d, J = 9.52 Hz, 1H) 7.79 (d, J = 8.79 Hz, 1H) 8.16 (d, J = 8.05 Hz, 1H) 9.15 (t, J = 5.12 Hz, 1H) | 479 |
| 140 | 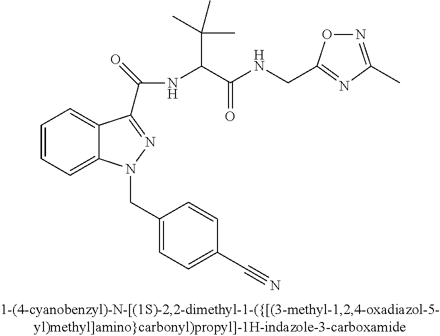 | ¹H NMR (400 MHz, DMSO-d6) δ ppm 0.98 (s, 9H) 2.28 (s, 3H) 4.46-4.69 (m, 3H) 5.82-6.00 (m, 2H) 7.22-7.40 (m, 3H) 7.47 (t, J = 7.69 Hz, 1H) 7.61 (d, J = 10.25 Hz, 1H) 7.71-7.85 (m, 3H) 8.18 (d, J = 8.05 Hz, 1H) 9.15 (t, J = 5.49 Hz, 1H) | 486 |
| 141 | 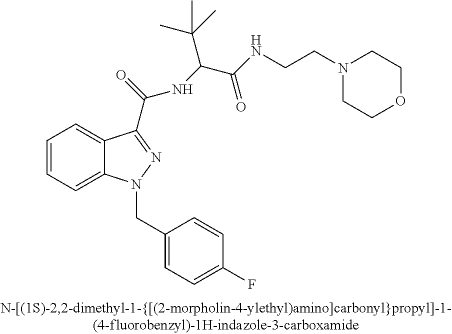 | ¹H NMR (400 MHz, DMSO-d6) δ ppm 0.98 (s, 9H) 2.34 (br. s., 6H) 3.07 (dd, J = 12.45, 5.86 Hz, 1H) 3.51 (br. s., 5H) 4.47 (d, J = 9.52 Hz, 1H) 5.78 (s, 2H) 7.15 (t, J = 8.79 Hz, 2H) 7.24-7.36 (m, 3H) 7.45 (t, J = 7.69 Hz, 1H) 7.61 (d, J = 9.52 Hz, 1H) 7.79 (d, J = 8.79 Hz, 1H) 8.16 (d, J = 8.05 Hz, 1H) 8.23 (br. s., 1H) | 496 |
| 142 | 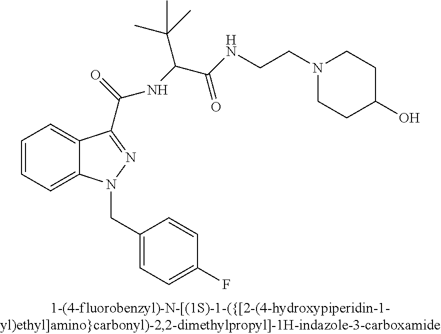 | ¹H NMR (400 MHz, DMSO-d6) δ ppm 0.97 (s, 9H) 1.38 (br. s., 2H) 1.67 (br. s., 2H) 2.49 (br. s., 2H) 2.79 (br. s., 2H) 3.12 (br. s., 1H) 3.44 (br. s., 4H) 4.46 (d, J = 9.52 Hz, 1H) 4.62 (br. s., 1H) 5.78 (s, 2H) 7.16 (t, J = 8.79 Hz, 2H) 7.24-7.36 (m, 3H) 7.46 (t, J = 7.69 Hz, 1H) 7.61 (d, J = 10.25 Hz, 1H) 7.79 (d, J = 8.05 Hz, 1H) 8.16 (d, J = 8.05 Hz, 1H) 8.20-8.37 (m, 1H) | 510 |
| 143 | 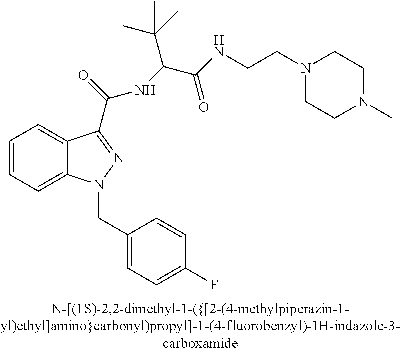 | ¹H NMR (400 MHz, DMSO-d6) δ ppm 0.97 (s, 9H) 2.13 (s, 3H) 2.33 (dd, J = 11.71, 5.86 Hz, 5H) 2.92-3.13 (m, 2H) 3.16-3.59 (m, 5H) 4.46 (d, J = 9.52 Hz, 1H) 5.67-5.87 (m, 2H) 7.16 (t, J = 8.79 Hz, 2H) 7.24-7.37 (m, 3H) 7.45 (t, J = 7.32 Hz, 1H) 7.60 (d, J = 10.25 Hz, 1H) 7.79 (d, J = 8.79 Hz, 1H) 8.07-8.26 (m, 2H) | 509 |
| 144 | 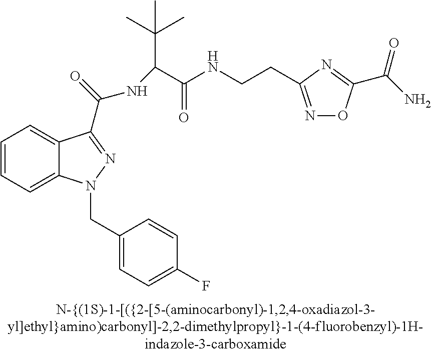 | ¹H NMR (400 MHz, DMSO-d6) δ ppm 0.92 (s, 9H) 2.94 (t, J = 6.96 Hz, 2H) 3.40 (ddd, J = 13.00, 6.41, 6.22 Hz, 1H) 3.60 (dq, J = 13.18, 6.59 Hz, 1H) 4.42 (d, J = 9.52 Hz, 1H) 5.78 (s, 2H) 7.16 (t, J = 8.79 Hz, 2H) 7.23-7.36 (m, 3H) 7.45 (t, J = 7.69 Hz, 1H) 7.58 (d, J = 9.52 Hz, 1H) 7.79 (d, J = 8.79 Hz, 1H) 8.15 (d, J = 8.05 Hz, 1H) 8.38 (s, 1H) 8.45 (t, J = 5.49 Hz, 1H) 8.69 (s, 1H) | 522 |
| 145 | 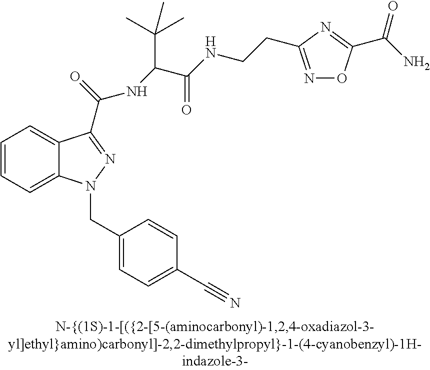 | ¹H NMR (400 MHz, DMSO-d6) δ ppm 0.83-1.01 (m, 9H) 2.94 (t, J = 6.59 Hz, 2H) 3.40 (dd, J = 13.18, 6.59 Hz, 1H) 3.51-3.67 (m, 1H) 4.42 (d, J = 9.52 Hz, 1H) 5.92 (s, 2H) 7.23-7.40 (m, 3H) 7.46 (t, J = 7.69 Hz, 1H) 7.58 (d, J = 9.52 Hz, 1H) 7.69-7.87 (m, 3H) 8.17 (d, J = 8.05 Hz, 1H) 8.38 (br. s., 1H) 8.45 (t, J = 5.49 Hz, 1H) 8.69 (br. s., 1H) | 529 |
| 146 | 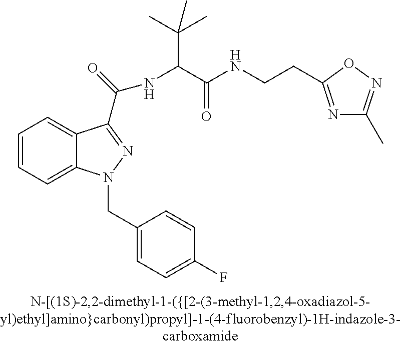 | ¹H NMR (400 MHz, DMSO-d6) δ ppm 0.91 (s, 9H) 2.24 (s, 3H) 2.95-3.14 (m, 2H) 3.38-3.51 (m, 1H) 3.51-3.69 (m, 1H) 4.42 (d, J = 9.52 Hz, 1H) 5.78 (s, 2H) 7.16 (t, J = 8.79 Hz, 2H) 7.23-7.39 (m, 3H) 7.45 (t, J = 7.69 Hz, 1H) 7.58 (d, J = 9.52 Hz, 1H) 7.79 (d, J = 8.05 Hz, 1H) 8.15 (d, J = 8.05 Hz, 1H) 8.46-8.59 (m, 1H) | 493 |
| 147 | 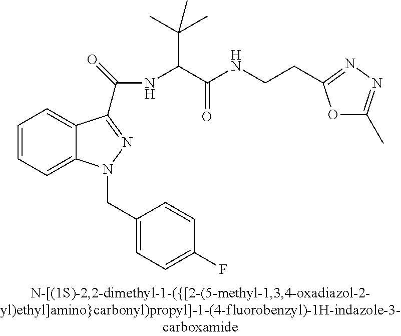 | ¹H NMR (400 MHz, DMSO-d6) δ ppm 0.92 (s, 9H) 2.38 (s, 3H) 2.96 (t, J = 6.59 Hz, 2H) 3.35-3.46 (m, 1H) 3.46-3.60 (m, 1H) 4.43 (d, J = 9.52 Hz, 1H) 5.78 (s, 2H) 7.16 (t, J = 8.79 Hz, 2H) 7.23-7.36 (m, 3H) 7.45 (t, J = 7.69 Hz, 1H) 7.58 (d, J = 9.52 Hz, 1H) 7.79 (d, J = 8.05 Hz, 1H) 8.16 (d, J = 8.05 Hz, 1H) 8.51 (t, J = 5.13 Hz, 1H) | 493 |
| 148 | 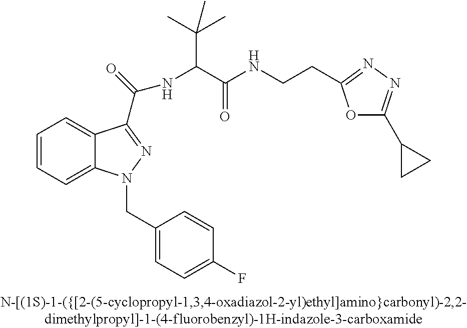 | ¹H NMR (400 MHz, DMSO-d6) δ ppm 0.79-0.97 (m, 11H) 0.95-1.14 (m, 2H) 1.97-2.19 (m, 1H) 2.93 (t, J = 6.22 Hz, 2H) 3.38 (d, J = 5.86 Hz, 1H) 3.45-3.64 (m, 1H) 4.42 (d, J = 9.52 Hz, 1H) 5.76 (s, 2H) 7.16 (t, J = 8.79 Hz, 2H) 7.24-7.38 (m, 3H) 7.41-7.52 (m, 1H) 7.57 (d, J = 9.52 Hz, 1H) 7.74-7.88 (m, 1H) 8.16 (d, J = 8.05 Hz, 1H) 8.49 (t, J = 5.49 Hz, 1H) | 519 |
| 149 | 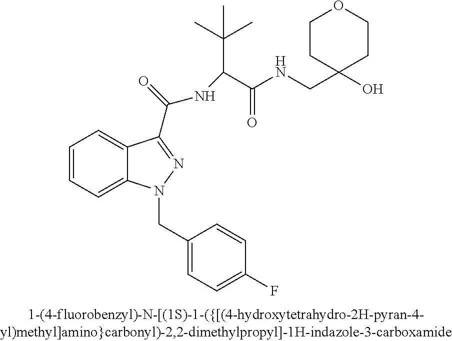 | ¹H NMR (400 MHz, DMSO-d6) δ ppm 0.98 (s, 9H) 1.33 (t, J = 11.35 Hz, 2H) 1.44-1.60 (m, 2H) 2.96-3.12 (m, 1H) 3.12-3.24 (m, 1H) 3.55 (d, J = 3.66 Hz, 4H) 4.49 (s, 1H) 4.61 (d, J = 9.52 Hz, 1H) 5.78 (s, 2H) 7.15 (t, J = 8.79 Hz, 2H) 7.22-7.38 (m, 3H) 7.45 (t, J = 7.69 Hz, 1H) 7.63 (d, J = 9.52 Hz, 1H) 7.79 (d, J = 8.79 Hz, 1H) 8.09-8.28 (m, 2H) | 497 |
| 150 | 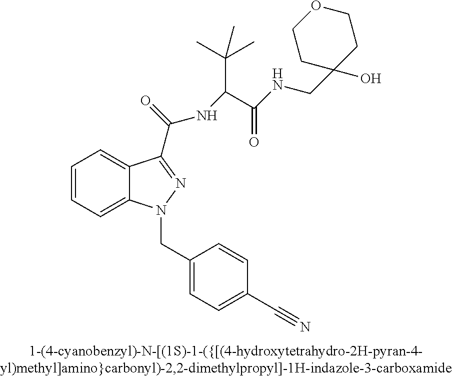 | ¹H NMR (400 MHz, DMSO-d6) δ ppm 0.97 (s, 9H) 1.33 (t, J = 10.98 Hz, 2H) 1.49 (ddd, J = 13.54, 7.32, 6.96 Hz, 2H) 2.99-3.10 (m, 1H) 3.13-3.23 (m, 1H) 3.56 (br. s., 4H) 4.48 (s, 1H) 4.61 (d, J = 10.25 Hz, 1H) 5.92 (s, 2H) 7.25-7.41 (m, 3H) 7.47 (t, J = 7.32 Hz, 1H) 7.62 (d, J = 9.52 Hz, 1H) 7.70-7.86 (m, 3H) 8.13-8.27 (m, 2H) | 504 |
| 151 | 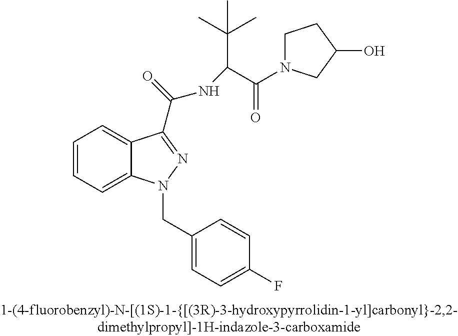 | ¹H NMR (400 MHz, DMSO-d6) δ ppm 0.99 (d, J = 6.59 Hz, 9H) 1.63-2.03 (m, 3H) 3.13-3.57 (m, 1H) 3.59-3.81 (m, 2H) 4.21-4.35 (m, 1H) 4.78 (dd, J = 16.84, 9.52 Hz, 1H) 4.89-5.09 (m, 1H) 5.68-5.85 (m, 2H) 7.15 (t, J = 8.79 Hz, 2H) 7.30 (q, J = 7.08 Hz, 3H) 7.45 (t, J = 7.69 Hz, 1H) 7.56 (t, J = 8.79 Hz, 1H) 7.79 (dd, J = 8.42, 4.03 Hz, 1H) 8.16 (dd, J = 8.05, 2.93 Hz, 1H) | 453 |
| 152 | 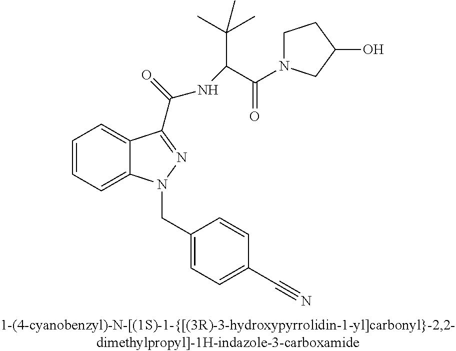 | ¹H NMR (400 MHz, DMSO-d6) δ ppm 0.99 (d, J = 5.86 Hz, 9H) 1.65-1.99 (m, 3H) 3.16-3.58 (m, 1H) 3.58-3.82 (m, 2H) 4.20-4.38 (m, 1H) 4.78 (dd, J = 16.11, 9.52 Hz, 1H) 4.89-5.12 (m, 1H) 5.88-5.95 (m, 2H) 7.25-7.41 (m, 3H) 7.47 (t, J = 7.69 Hz, 1H) 7.56 (t, J = 8.79 Hz, 1H) 7.68-7.87 (m, 3H) 8.18 (dd, J = 8.05, 2.93 Hz, 1H) | 460 |
| 153 | 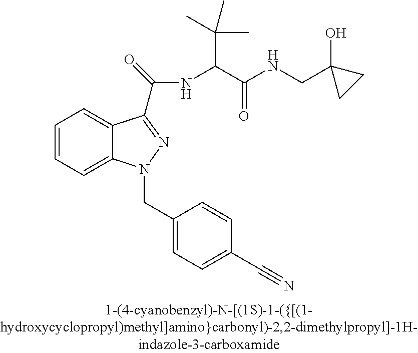 | ¹H NMR (400 MHz, DMSO-d6) δ ppm 0.91 (t, J = 7.32 Hz, 3H) 0.94 (br. s., 1H) 0.99 (s, 9H) 2.42 (q, J = 7.32 Hz, 2H) 3.92-4.02 (m, 2H) 4.58 (d, J = 9.88 Hz, 1H) 5.87-5.96 (m, 2H) 7.28-7.38 (m, 3H) 7.47 (t, J = 7.69 Hz, 1H) 7.60 (d, J = 9.52 Hz, 1H) 7.76 (d, J = 8.79 Hz, 1H) 7.79 (d, J = 8.42 Hz, 2H) 8.18 (d, J = 8.05 Hz, 1H) 8.56 (t, J = 5.49 Hz, 1H) | 460.55 |
| 154 | 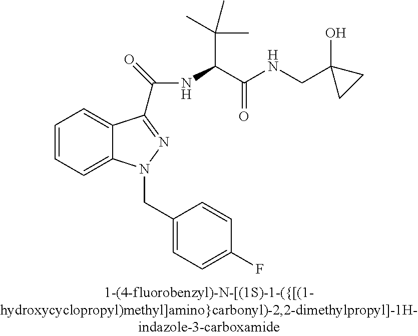 | ¹H NMR (400 MHz, DMSO-d6) δ ppm 0.91 (t, J = 7.32 Hz, 3H) 0.95 (br. s., 1H) 1.00 (s, 9H) 2.42 (q, J = 7.32 Hz, 2H) 3.95-4.03 (m, 1H) 4.58 (d, J = 9.88 Hz, 1H) 5.77 (s, 2H) 7.15 (t, J = 8.79 Hz, 2H) 7.26-7.33 (m, 3H) 7.45 (t, J = 7.69 Hz, 1H) 7.60 (d, J = 9.52 Hz, 1H) 7.79 (d, J = 8.42 Hz, 1H) 8.16 (d, J = 8.05 Hz, 1H) 8.56 (t, J = 5.49 Hz, 1H) | 451.53 |
| 155 | 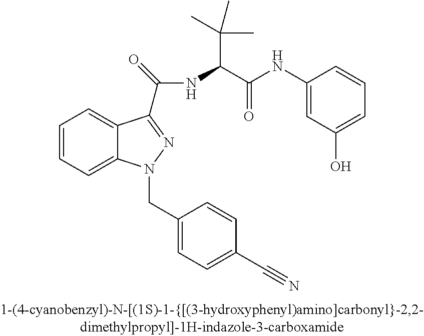 | ¹H NMR (400 MHz, DMSO-d6) δ ppm 1.02 (s, 9H) 4.71 (d, J = 9.52 Hz, 1H) 5.93 (s, 2H) 6.46 (d, J = 7.69 Hz, 1H) 6.95-7.00 (m, 1H) 7.07 (t, J = 8.05 Hz, 1H) 7.17 (s, 1H) 7.29-7.39 (m, 3H) 7.47 (t, J = 7.69 Hz, 1H) 7.66 (d, J = 9.52 Hz, 1H) 7.77 (d, J = 8.79 Hz, 1H) 7.80 (d, J = 8.05 Hz, 2H) 8.19 (d, J = 8.05 Hz, 1H) 9.42 (s, 1H) 10.20 (s, 1H) | 482.56 |
| 156 | 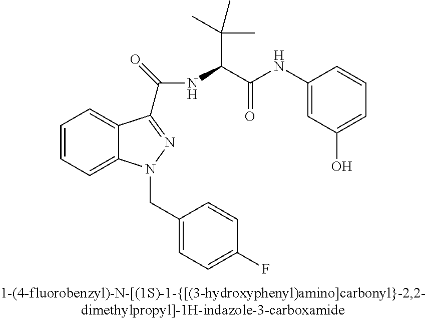 | ¹H NMR (400 MHz, DMSO-d6) δ ppm 0.98 (br. s., 1H) 1.03 (s, 9H) 4.71 (d, J = 9.52 Hz, 1H) 5.79 (s, 2H) 6.46 (dd, J = 8.05, 1.46 Hz, 1H) 6.98 (d, J = 8.05 Hz, 1H) 7.07 (t, J = 8.05 Hz, 1H) 7.12-7.19 (m, 3H) 7.26-7.35 (m, 3H) 7.46 (t, J = 7.69 Hz, 1H) 7.66 (d, J = 9.52 Hz, 1H) 7.79 (d, J = 8.42 Hz, 1H) 8.17 (d, J = 8.42 Hz, 1H) 9.42 (s, 1H) 10.20 (s, 1H) | 475.54 |
| 157 | 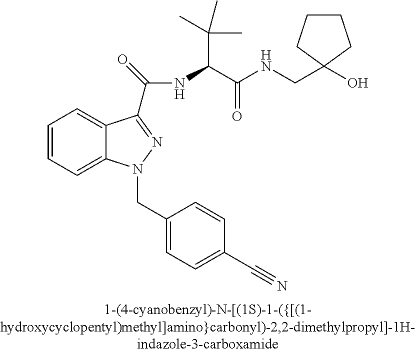 | ¹H NMR (400 MHz, DMSO-d6) δ ppm 0.96 (s, 9H) 1.51 (d, J = 1.83 Hz, 2H) 1.56 (br. s., 1H) 1.59-1.68 (m, 3H) 1.77 (d, J = 5.49 Hz, 1H) 1.89 (d, J = 5.49 Hz, 1H) 3.39 (dd, J = 10.43, 5.67 Hz, 1H) 3.54 (dd, J = 10.62, 5.49 Hz, 1H) 4.54 (d, J = 9.88 Hz, 1H) 4.76 (t, J = 5.49 Hz, 1H) 5.92 (s, 2H) 7.28-7.38 (m, 3H) 7.47 (t, J = 7.69 Hz, 1H) 7.58 (d, J = 9.88 Hz, 1H) 7.75-7.86 (m, 4H) 8.19 (d, J = 8.05 Hz, 1H) | 488.6 |
| 158 | 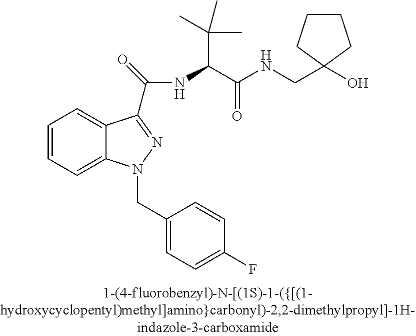 | ¹H NMR (400 MHz, DMSO-d6) δ ppm 0.96 (s, 9H) 1.51 (br. s., 2H) 1.56 (br. s., 1H) 1.59-1.68 (m, 3H) 1.77 (d, J = 5.86 Hz, 1H) 1.89 (d, J = 5.12 Hz, 1H) 3.39 (dd, J = 10.43, 5.67 Hz, 1H) 3.55 (dd, J = 10.43, 5.67 Hz, 1H) 4.54 (d, J = 9.88 Hz, 1H) 4.76 (t, J = 5.49 Hz, 1H) 5.78 (s, 2H) 7.16 (t, J = 8.79 Hz, 2H) 7.26-7.35 (m, 3H) 7.45 (t, J = 7.69 Hz, 1H) 7.59 (d, J = 9.88 Hz, 1H) 7.79 (d, J = 8.42 Hz, 1H) 7.85 (s, 1H) 8.17 (d, J = 8.05 Hz, 1H) | 481.58 |
| 159 | 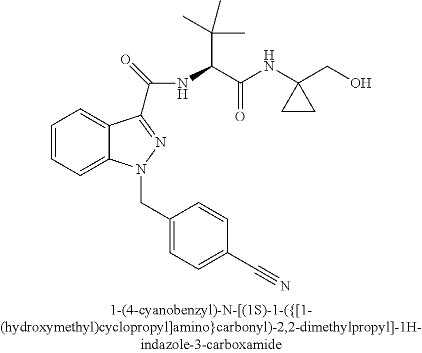 | ¹H NMR (400 MHz, DMSO-d6) δ ppm 0.43-0.63 (m, 2H) 0.68 (br. s., 2H) 0.93 (s, 9H) 2.49-2.59 (m, 1H) 3.40-3.51 (m, 1H) 4.43 (d, J = 9.52 Hz, 1H) 4.69 (t, J = 5.86 Hz, 1H) 5.92 (s, 2H) 7.21-7.40 (m, 3H) 7.46 (t, J = 7.69 Hz, 1H) 7.59 (d, J = 9.52 Hz, 1H) 7.72-7.89 (m, 3H) 8.18 (d, J = 8.05 Hz, 1H) 8.54 (s, 1H) | 460.55 |
| 160 | 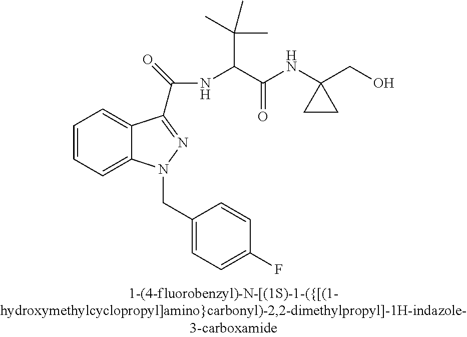 | ¹H NMR (400 MHz, DMSO-d6) δ ppm 0.50-0.63 (m, 1H) 0.69 (br. s., 2H) 0.94 (s, 9H) 1.46-1.86 (m, 2H) 1.94 (quin, J = 7.32 Hz, 2H) 2.52-2.63 (m, 1H) 3.42-3.55 (m, 1H) 4.42 (d, J = 9.52 Hz, 1H) 4.52-4.63 (m, 2H) 4.69 (t, J = 5.49 Hz, 1H) 7.28 (t, J = 7.32 Hz, 1H) 7.45-7.66 (m, 2H) 7.81 (d, J = 8.79 Hz, 1H) 8.15 (d, J = 8.05 Hz, 1H) 8.54 (s, 2H) | 453.53 |
| 161 | 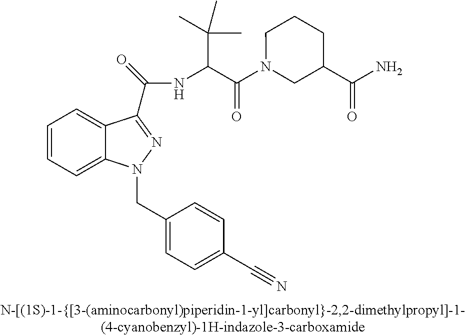 | ¹H NMR (400 MHz, DMSO-d6) δ ppm 0.93-1.02 (m, 9H) 1.44 (s, 2H) 1.57 (br. s., 2H) 1.76 (br. s., 2H) 2.15 (d, J = 10.62 Hz, 1H) 3.05 (br. s., 1H) 3.13 (br. s., 1H) 4.14 (br. s., 1H) 4.43 (br. s., 1H) 5.06-5.12 (m, 1H) 5.91 (d, J = 1.83 Hz, 2H) 6.82 (br. s., 1H) 7.28-7.39 (m, 4H) 7.45-7.49 (m, 1H) 7.61 (t, J = 8.79 Hz, 1H) 7.74-7.82 (m, 4H) 8.15-8.20 (m, 1H) | 501.6 |
| 162 | 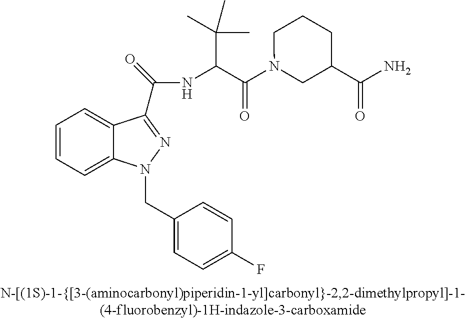 | ¹H NMR (400 MHz, DMSO-d6) δ ppm 0.94 (m, 9H) 1.03 (bs, 2H) 1.55 (d, J = 12.45 Hz, 1H) 1.75 (d, J = 2.93 Hz, 1H) 1.90 (br. s., 1H) 2.15 (d, J = 10.98 Hz, 1H) 3.09 (br. s., 1H) 4.14 (br. s., 1H) 4.44 (br. s., 1H) 5.06-5.11 (m, 1H) 5.77 (br. s., 2H) 7.12-7.19 (m, 2H) 7.26-7.38 (m, 4H) 7.38-7.48 (m, 2H) 7.58-7.67 (m, 1H) 7.77-7.82 (m, 1H) 8.16 (d, J = 8.05 Hz, 1H) | 494.58 |
| 163 | 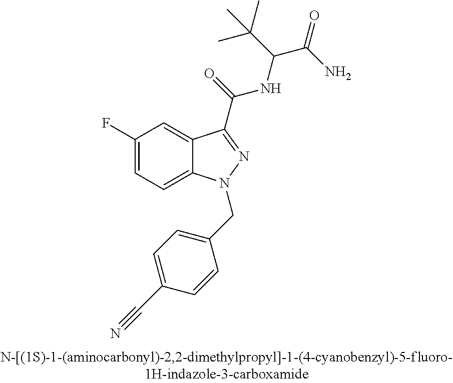 | ¹H NMR (400 MHz, DMSO-d6) δ ppm 7.78-7.88 (m, 3H), 7.71-7.76 (m, 1H), 7.56-7.61 (m, J = 9.52 Hz, 1H), 7.38-7.45 (m, 1H), 7.34-7.38 (m, J = 8.05 Hz, 2H), 7.26-7.31 (m, 1H), 5.92-5.95 (m, 2H), 4.45 (d, J = 9.52 Hz, 1H), 0.98 (s, 9H) | 408 |
| 164 | 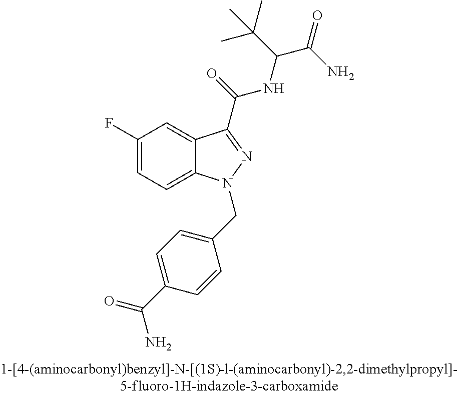 | ¹H NMR (400 MHz, DMSO-d6) δ ppm 7.93 (br. s., 1H), 7.83-7.88 (m, 1H), 7.81 (d, J = 8.42 Hz, 2H), 7.74 (br. s., 1H), 7.60 (d, J = 9.88 Hz, 1H), 7.33-7.44 (m, 2H), 7.25-7.32 (m, 3H), 5.87 (s, 2H), 4.45 (d, J = 9.52 Hz, 1H), 0.97 (s, 9H) | 426 |
| 165 | 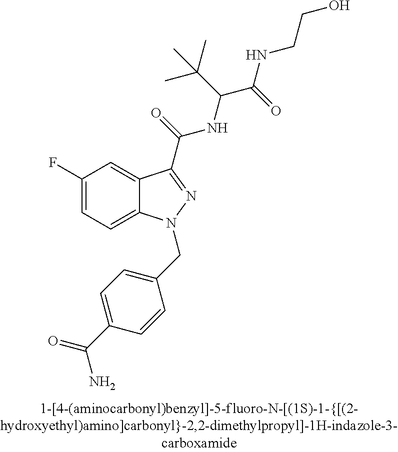 | ¹H NMR (400 MHz, DMSO-d6) δ ppm 8.32 (t, J = 5.31 Hz, 1H), 7.93 (br. s., 1H), 7.83-7.89 (m, 1H), 7.81 (d, J = 8.05 Hz, 2H), 7.60 (d, J = 9.52 Hz, 1H), 7.33-7.45 (m, 2H), 7.29 (d, J = 8.05 Hz, 2H), 5.87 (s, 2H), 4.68 (t, J = 5.31 Hz, 1H), 3.38-3.45 (m, 2H), 3.05-3.26 (m, 2H), 0.96 (s, 9H) | 470 |
| 166 | 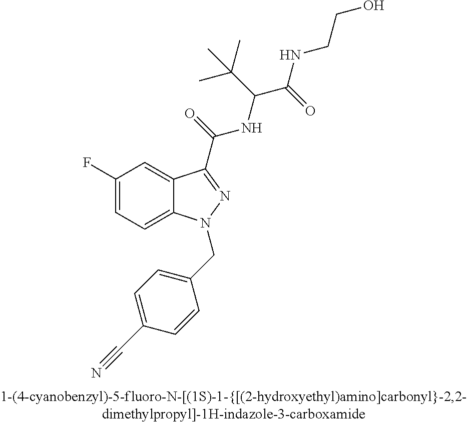 | ¹H NMR (400 MHz, DMSO-d6) δ ppm 8.32 (t, J = 5.49 Hz, 1H), 7.83-7.89 (m, 1H), 7.81 (d, J = 8.05 Hz, 2H), 7.59 (d, J = 9.88 Hz, 1H), 7.38-7.45 (m, 1H), 7.36 (d, J = 8.05 Hz, 2H), 5.94 (s, 2H), 4.68 (t, J = 5.31 Hz, 1H), 4.49 (d, J = 9.52 Hz, 1H), 3.38-3.45 (m, 2H), 3.06-3.26 (m, 2H), 0.96 (s, 9H) | 452 |
| 167 | 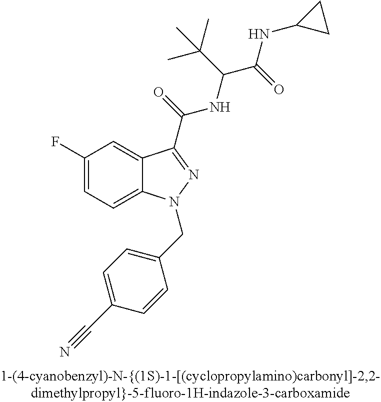 | ¹H NMR (400 MHz, DMSO-d6) δ ppm 8.37 (d, J = 4.03 Hz, 1H), 7.83-7.90 (m, 1H), 7.82 (d, J = 8.42 Hz, 2H), 7.56 (d, J = 9.88 Hz, 1H), 7.40-7.45 (m, 1H), 7.34-7.40 (m, 2H), 5.94 (s, 2H), 4.39 (d, J = 9.52 Hz, 1H), 2.60-2.70 (m, 1H), 0.94 (s, 9H), 0.57-0.66 (m, 2H), 0.35-0.45 (m, 2H) | 448 |
| 168 | 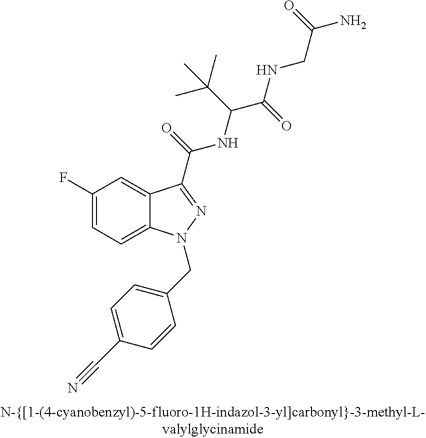 | ¹H NMR (400 MHz, DMSO-d6) δ ppm 8.49 (t, J = 5.67 Hz, 1H), 7.77-7.89 (m, 4H), 7.62 (d, J = 9.15 Hz, 1H), 7.29-7.46 (m, 4H), 6.97-7.05 (m, 1H), 5.94 (s, 2H), 4.53 (d, J = 9.52 Hz, 1H), 3.65-3.72 (m, 2H), 0.98 (s, 9H) | 465 |
| 169 | 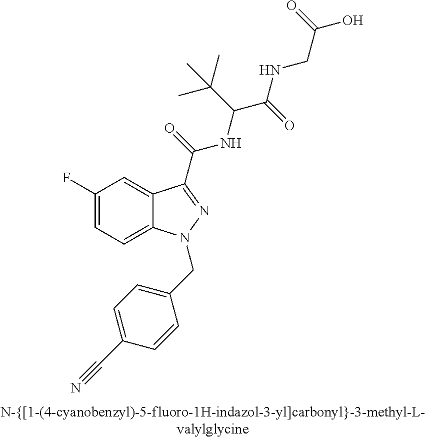 | ¹H NMR (400 MHz, DMSO-d6) δ ppm 8.67 (t, J = 5.67 Hz, 1H), 7.83-7.89 (m, 1H), 7.81 (d, J = 8.05 Hz, 2H), 7.60 (d, J = 9.52 Hz, 1H), 7.38-7.45 (m, 1H), 7.37 (d, J = 8.05 Hz, 2H), 5.94 (s, 2H), 4.55 (d, J = 9.88 Hz, 1H), 3.70-3.90 (m, 2H), 0.95-1.04 (m, 9H) | 466 |
| 170 | 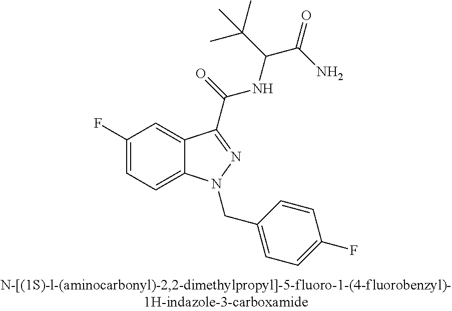 | ¹H NMR (400 MHz, DMSO-d6) δ ppm 7.85-7.90 (m, 1H) 7.76-7.82 (m, 1H) 7.73 (br. s., 1H) 7.58 (d, J = 9.52 Hz, 1H) 7.35-7.43 (m, 1H) 7.25-7.35 (m, 3H) 7.11-7.20 (m, 2H) 5.79 (s, 2H) 4.44 (d, J = 9.52 Hz, 1H) 0.97 (s, 9H) | 401 |
| 171 | 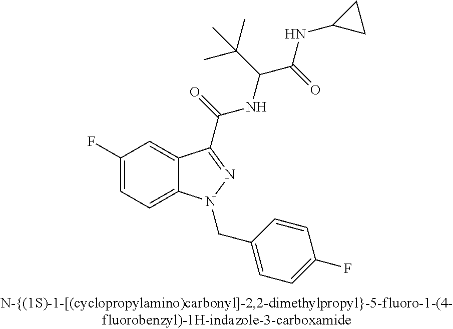 | ¹H NMR (400 MHz, DMSO-d6) δ ppm 8.36 (d, J = 4.03 Hz, 1H), 7.83-7.91 (m, 1H), 7.73-7.83 (m, 1H), 7.55 (d, J = 9.52 Hz, 1H), 7.35-7.45 (m, 1H), 7.26-7.35 (m, 2H), 7.16 (t, J = 8.97 Hz, 2H), 5.79 (s, 2H), 4.38 (d, J = 9.88 Hz, 1H), 2.59-2.71 (m, 1H), 0.94 (s, 9H), 0.56-0.66 (m, 2H), 0.34-0.45 (m, 2H) | 441 |
| 172 | 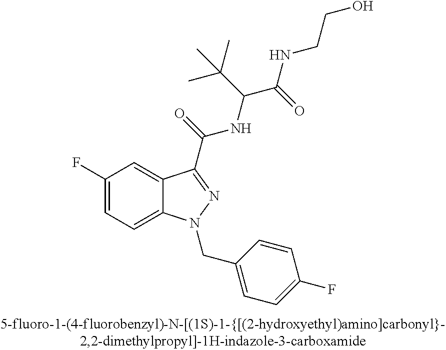 | ¹H NMR (400 MHz, DMSO-d6) δ ppm 8.31 (t, J = 5.49 Hz, 1H), 7.82-7.91 (m, 1H), 7.75-7.82 (m, 1H), 7.59 (d, J = 9.88 Hz, 1H), 7.35-7.43 (m, 1H), 7.28-7.35 (m, 2H), 7.16 (t, J = 8.79 Hz, 2H), 5.79 (s, 2H), 4.68 (t, J = 5.31 Hz, 1H), 4.48 (d, J = 9.52 Hz, 1H), 3.40 (q, J = 5.86 Hz, 2H), 3.06-3.25 (m, 2H), 0.95 (s, 9H) | 445 |
| 173 | 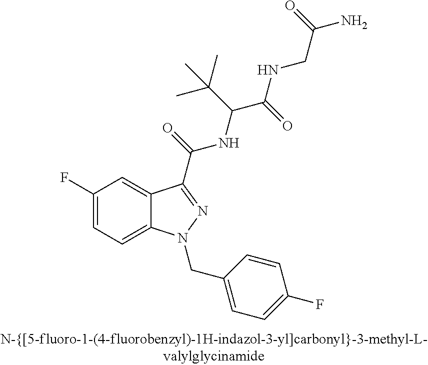 | ¹H NMR (400 MHz, DMSO-d6) δ ppm 8.48 (t, J = 5.49 Hz, 1H), 7.82-7.90 (m, 1H), 7.75-7.82 (m, 1H), 7.61 (d, J = 9.52 Hz, 1H), 7.35-7.43 (m, 1H), 7.28-7.35 (m, 2H), 7.16 (t, J = 8.97 Hz, 2H), 7.00 (br. s., 1H), 5.79 (s, 2H), 4.52 (d, J = 9.52 Hz, 1H), 3.64-3.71 (m, 2H), 0.98 (s, 9H) | 458 |
| 174 | 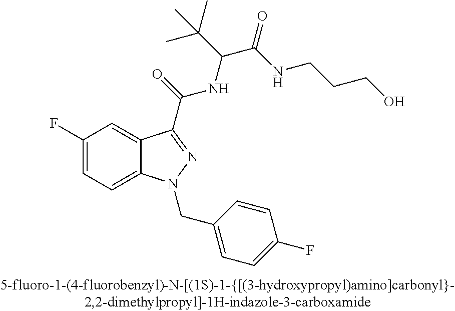 | ¹H NMR (400 MHz, DMSO-d6) δ ppm 8.28 (t, J = 5.49 Hz, 1H), 7.83-7.92 (m, 1H), 7.75-7.82 (m, 1H), 7.57 (d, J = 9.88 Hz, 1H), 7.35-7.43 (m, 1H), 7.27-7.35 (m, 2H), 7.16 (t, J = 8.97 Hz, 2H), 5.79 (s, 2H), 4.39-4.49 (m, 2H), 3.37-3.45 (m, 2H), 2.98-3.26 (m, 2H), 1.48-1.62 (m, 2H), 0.96 (s, 9H) | 459 |
| 175 | 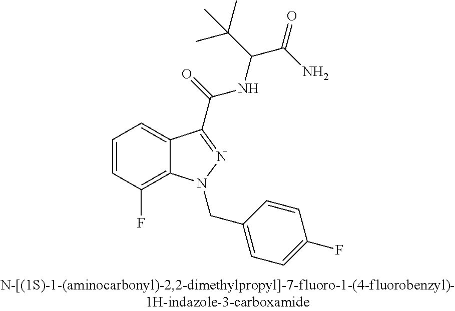 | ¹H NMR (400 MHz, DMSO-d6) δ ppm 7.99 (d, J = 7.69 Hz, 1H), 7.73 (br. s., 1H), 7.63 (d, J = 9.52 Hz, 1H), 7.20-7.34 (m, 4H), 7.16 (t, J = 8.79 Hz, 2H), 5.80 (s, 2H), 4.45 (d, J = 9.88 Hz, 1H), 0.98 (s, 9H) | 401 |
| 176 | 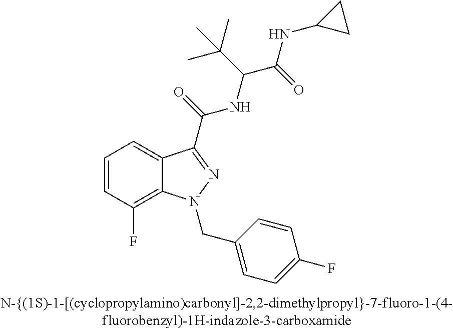 | ¹H NMR (400 MHz, DMSO-d6) δ ppm 8.36 (d, J = 4.03 Hz, 1H), 7.98 (d, J = 7.69 Hz, 1H), 7.61 (d, J = 9.52 Hz, 1H), 7.20-7.35 (m, 3H), 7.16 (t, J = 8.79 Hz, 2H), 5.80 (s, 2H), 4.40 (d, J = 9.52 Hz, 1H), 2.59-2.73 (m, 1H), 0.94 (s, 9H), 0.58-0.67 (m, 2H), 0.35-0.45 (m, 2H) | 441 |
| 177 | 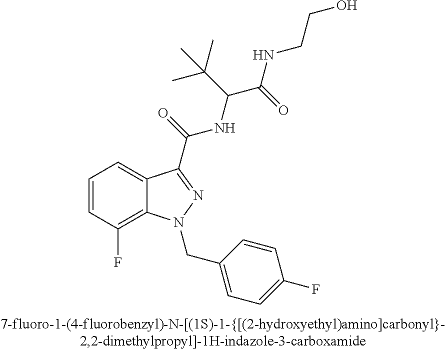 | ¹H NMR (400 MHz, DMSO-d6) δ ppm 8.32 (t, J = 5.49 Hz, 1H), 7.99 (d, J = 7.69 Hz, 1H), 7.64 (d, J = 9.88 Hz, 1H), 7.20-7.34 (m, 3H), 7.16 (t, J = 8.79 Hz, 2H), 5.79 (s, 2H), 4.68 (t, J = 5.31 Hz, 1H), 4.50 (d, J = 9.88 Hz, 1H), 3.41 (q, J = 5.98 Hz, 2H), 3.04-3.25 (m, 2H), 0.96 (s, 9H) | 445 |
| 178 | 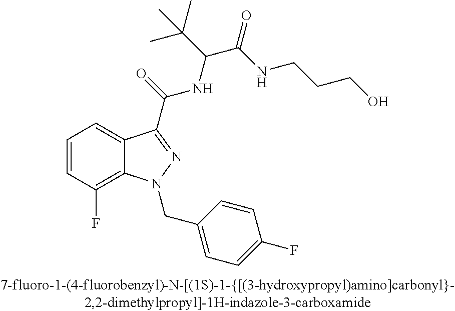 | ¹H NMR (400 MHz, DMSO-d6) δ ppm 8.28 (t, J = 5.31 Hz, 1H), 7.99 (d, J = 7.69 Hz, 1H), 7.63 (d, J = 9.52 Hz, 1H), 7.20-7.35 (m, 3H), 7.16 (t, J = 8.79 Hz, 2H), 5.79 (s, 2H), 4.40-4.50 (m, 2H), 3.40 (q, J = 6.22 Hz, 2H), 2.99-3.27 (m, 2H), 1.52-1.62 (m, 2H), 0.96 (s, 9H) | 459 |
| 179 | 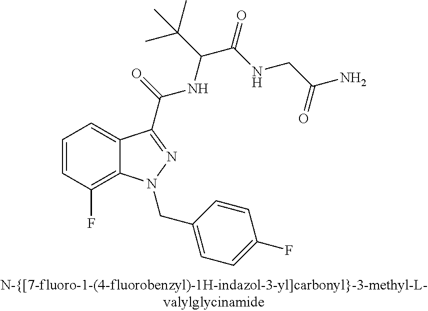 | ¹H NMR (400 MHz, DMSO-d6) δ ppm 8.49 (t, J = 5.67 Hz, 1H), 7.99 (d, J = 7.69 Hz, 1H), 7.68 (d, J = 9.52 Hz, 1H), 7.20-7.36 (m, 4H), 7.16 (t, J = 8.79 Hz, 2H), 7.01 (br. s., 1H), 5.79 (s, 2H), 4.54 (d, J = 9.52 Hz, 1H), 3.65-3.72 (m, 2H), 0.99 (s, 9H) | 458 |
| 180 | 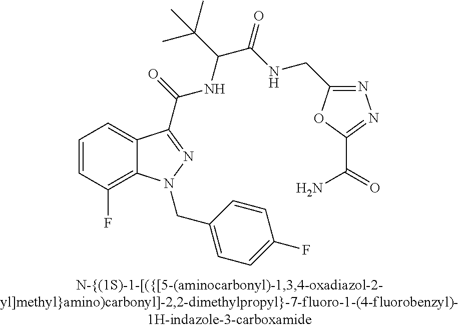 | ¹H NMR (400 MHz, DMSO-d6) δ ppm 9.16 (t, J = 5.31 Hz, 1H) 8.59 (s, 1H) 8.19 (s, 1H) 7.98 (d, J = 8.05 Hz, 1H) 7.66 (d, J = 9.88 Hz, 1H) 7.20-7.35 (m, 3H) 7.11-7.20 (m, 2H) 5.79 (s, 2H) 4.53-4.71 (m, 3H) 0.98 (s, 9H) | 526 |
| 181 | 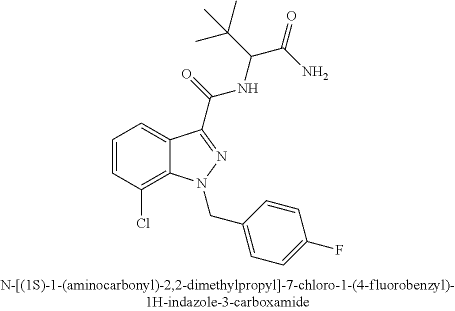 | ¹H NMR (400 MHz, DMSO-d6) δ ppm 8.21 (d, J = 8.05 Hz, 1H) 7.74 (br. s., 1H) 7.65 (d, J = 9.52 Hz, 1H) 7.55 (d, J = 7.32 Hz, 1H) 7.25-7.34 (m, 2H) 7.08-7.19 (m, 3H) 6.04 (d, J = 6.59 Hz, 2H) 4.46 (d, J = 9.52 Hz, 1H) 0.98 (s, 9H) | 417 |
| 182 | 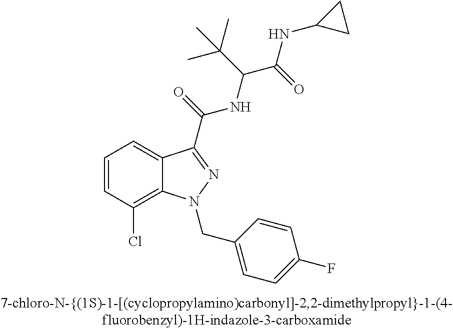 | ¹H NMR (400 MHz, DMSO-d6) δ ppm 8.32-8.39 (m, 1H) 8.20 (d, J = 8.05 Hz, 1H) 7.62 (d, J = 9.88 Hz, 1H) 7.55 (d, J = 7.32 Hz, 1H) 7.29 (t, J = 7.87 Hz, 1H) 7.10-7.19 (m, 4H) 6.00-6.07 (m, 2H) 4.40 (d, J = 9.88 Hz, 1H) 2.60-2.71 (m, 1H) 0.94 (s, 9H) 0.58-0.65 (m, 2H) 0.33-0.44 (m, 2H) | 457 |
| 183 | 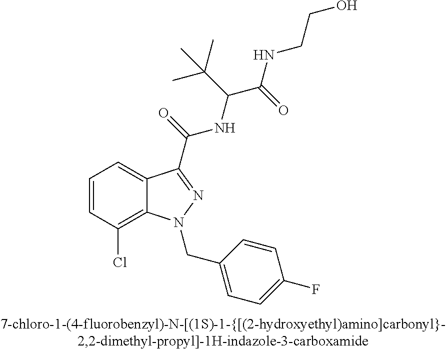 | ¹H NMR (400 MHz, DMSO-d6) δ ppm 8.32 (t, J = 5.31 Hz, 1H) 8.21 (d, J = 8.05 Hz, 1H) 7.66 (d, J = 9.52 Hz, 1H) 7.55 (d, J = 7.69 Hz, 1H) 7.29 (t, J = 7.69 Hz, 1H) 7.08-7.19 (m, 4H) 6.04 (d, J = 5.86 Hz, 2H) 4.63-4.72 (m, 1H) 4.50 (d, J = 9.52 Hz, 1H) 3.40 (d, J = 5.86 Hz, 2H) 3.03-3.25 (m, 2H) 0.96 (s, 9H) | 461 |
| 184 | 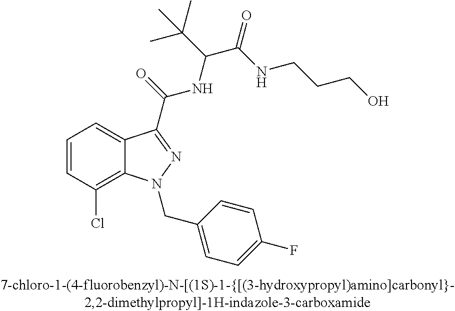 | ¹H NMR (400 MHz, DMSO-d6) δ ppm 8.28 (t, J = 5.31 Hz, 1H) 8.21 (d, J = 8.05 Hz, 1H) 7.65 (d, J = 9.88 Hz, 1H) 7.55 (d, J = 7.32 Hz, 1H) 7.29 (t, J = 7.87 Hz, 1H) 7.09-7.18 (m, 4H) 6.04 (d, J = 5.49 Hz, 1H) 4.41-4.49 (m, 2H) 3.40 (q, J = 6.22 Hz, 2H) 2.98-3.25 (m, 2H) 1.50-1.61 (m, 2H) 0.96 (s, 9H) | 475 |
| 185 | 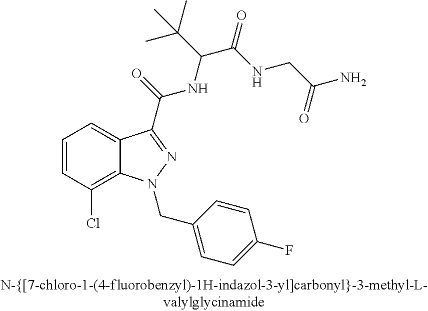 | ¹H NMR (400 MHz, DMSO-d6) δ ppm 8.49 (t, J = 5.67 Hz, 1H) 8.20 (d, J = 8.42 Hz, 1H) 7.69 (d, J = 9.52 Hz, 1H) 7.55 (d, J = 7.32 Hz, 1H) 7.33 (br. s., 1H) 7.29 (t, J = 7.87 Hz, 1H) 7.08-7.20 (m, 3H) 7.01 (br. s., 1H) 6.00-6.07 (m, 2H) 4.54 (d, J = 9.52 Hz, 1H) 3.64-3.71 (m, 2H) 0.98 (s, 9H) | 474 |
| 186 | 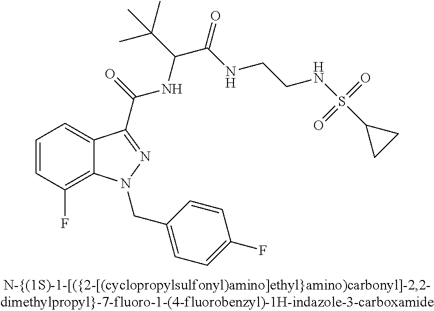 | ¹H NMR (400 MHz, DMSO-d6) δ ppm 8.41 (t, J = 5.49 Hz, 1H), 7.99 (d, J = 8.05 Hz, 1H), 7.64 (d, J = 9.52 Hz, 1H), 7.20-7.36 (m, 4H), 7.07-7.20 (m, 3H), 5.80 (s, 2H), 4.46 (d, J = 9.52 Hz, 1H), 3.13-3.28 (m, 2H), 2.96-3.10 (m, 2H), 0.97 (s, 9H), 0.79-0.93 (m, 4H) | 548 |
| 187 | 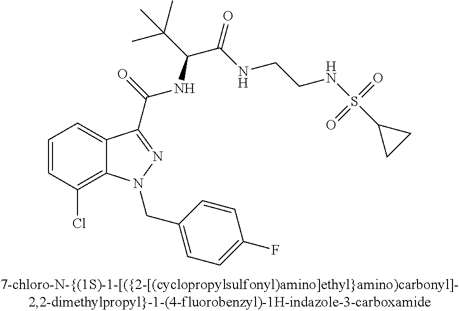 | ¹H NMR (400 MHz, DMSO-d6) δ ppm 8.41 (t, J = 5.67 Hz, 1H), 8.20 (d, J = 7.69 Hz, 1H), 7.65 (d, J = 9.52 Hz, 1H), 7.55 (d, J = 7.32 Hz, 1H), 7.29 (t, J = 7.69 Hz, 1H), 7.08-7.19 (m, 5H), 6.04 (d, J = 5.86 Hz, 2H), 4.46 (d, J = 9.52 Hz, 1H), 3.14-3.27 (m, 2H), 2.97-3.07 (m, 2H), 0.97 (s, 9H), 0.79-0.92 (m, 4H) | 564 |
| 188 | 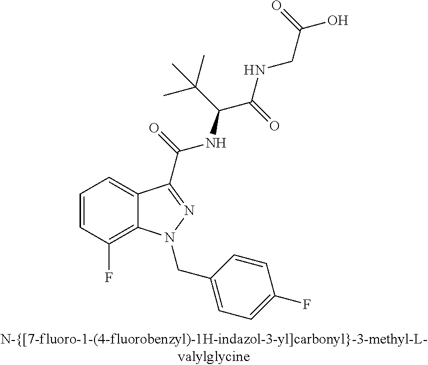 | ¹H NMR (400 MHz, DMSO-d6) δ ppm 8.66 (t, J = 5.86 Hz, 1H), 7.99 (d, J = 7.69 Hz, 1H), 7.65 (d, J = 9.88 Hz, 1H), 7.20-7.35 (m, 4H), 7.10-7.20 (m, 2H), 5.79 (s, 2H), 4.56 (d, J = 9.52 Hz, 1H), 3.68-3.90 (m, 2H), 0.99 (s, 9H) | 459 |
| 189 | 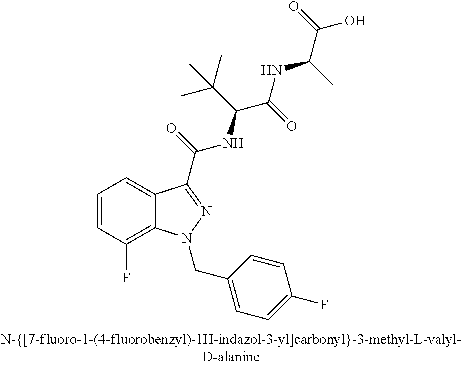 | ¹H NMR (400 MHz, DMSO-d6) δ ppm 8.67 (d, J = 7.32 Hz, 1H), 8.00 (d, J = 7.69 Hz, 1H), 7.65 (d, J = 9.88 Hz, 1H), 7.20-7.34 (m, 4H), 7.11-7.20 (m, 2H), 5.80 (s, 2H), 4.60 (d, J = 9.88 Hz, 1H), 4.21-4.31 (m, 1H), 1.27 (d, J = 7.32 Hz, 3H), 0.97 (s, 9H) | 473 |
| 190 | 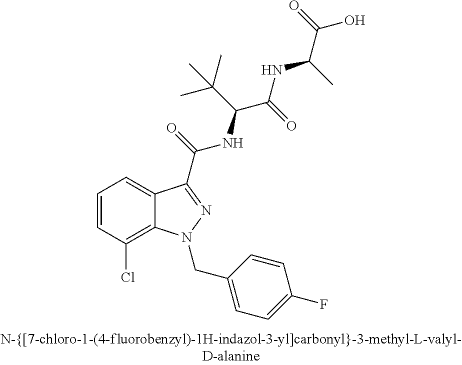 | ¹H NMR (400 MHz, DMSO-d6) δ ppm 8.67 (d, J = 7.32 Hz, 1H), 8.21 (d, J = 8.05 Hz, 1H), 7.66 (d, J = 9.88 Hz, 1H), 7.55 (d, J = 7.32 Hz, 1H), 7.29 (t, J = 7.87 Hz, 1H), 7.08-7.18 (m, 4H), 6.04 (s, 2H), 4.60 (d, J = 9.88 Hz, 1H), 4.22-4.31 (m, 1H), 1.27 (d, J = 7.32 Hz, 3H), 0.96 (s, 9H) | 489 |
| 191 | 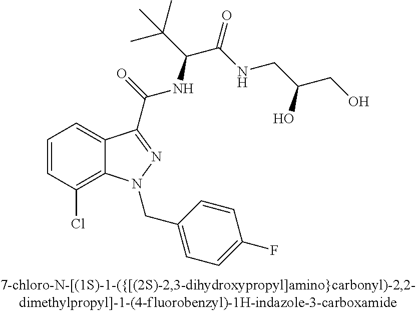 | ¹H NMR (400 MHz, DMSO-d6) δ ppm 8.27 (t, J = 5.49 Hz, 1H), 8.21 (d, J = 8.05 Hz, 1H), 7.67 (d, J = 9.52 Hz, 1H), 7.54 (d, J = 6.95 Hz, 1H), 7.29 (t, J = 7.87 Hz, 1H), 7.10-7.18 (m, 4H), 6.03 (d, J = 5.12 Hz, 2H), 4.72 (d, J = 4.76 Hz, 1H), 4.51-4.59 (m, 2H), 3.45-3.55 (m, 1H), 3.24-3.30 (m, 2H), 2.89-3.00 (m, 1H), 0.96 (s, 9H) | 491 |
| 192 | 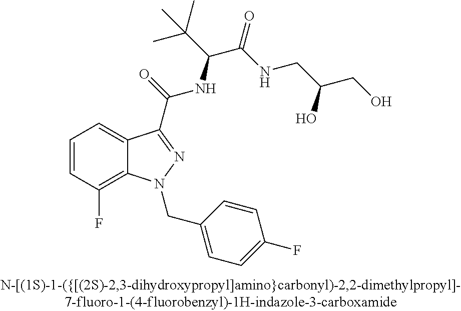 | ¹H NMR (400 MHz, DMSO-d6) δ ppm 8.27 (t, J = 5.67 Hz, 1H), 7.99 (d, J = 7.69 Hz, 1H), 7.66 (d, J = 9.88 Hz, 1H), 7.20-7.35 (m, 4H), 7.12-7.20 (m, 2H), 5.79 (s, 2H), 4.72 (d, J = 4.76 Hz, 1H), 4.51-4.58 (m, 2H), 3.45-3.55 (m, 1H), 3.24-3.30 (m, 3H), 2.91-3.00 (m, 1H), 0.97 (s, 9H) | 475 |
| 193 | 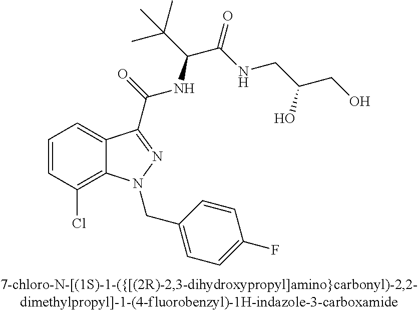 | ¹H NMR (400 MHz, DMSO-d6) δ ppm 8.28 (t, J = 5.49 Hz, 1H), 8.21 (d, J = 8.05 Hz, 1H), 7.67 (d, J = 9.52 Hz, 1H), 7.54 (d, J = 7.32 Hz, 1H), 7.29 (t, J = 7.87 Hz, 1H), 7.10-7.17 (m, 4H), 6.03 (d, J = 4.76 Hz, 2H), 4.74 (d, J = 5.12 Hz, 1H), 4.50-4.58 (m, 2H), 3.42-3.52 (m, 1H), 3.24-3.31 (m, 2H), 3.12-3.22 (m, 1H), 3.01-3.12 (m, 1H), 0.96 (s, 9H) | 491 |
| 194 | 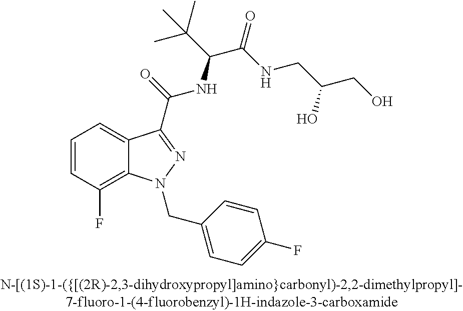 | ¹H NMR (400 MHz, DMSO-d6) δ ppm 8.28 (t, J = 5.49 Hz, 1H), 7.99 (d, J = 7.69 Hz, 1H), 7.65 (d, J = 9.52 Hz, 1H), 7.20-7.34 (m, 4H), 7.12-7.20 (m, 2H), 5.79 (s, 2H), 4.74 (d, J = 4.76 Hz, 1H), 4.49-4.57 (m, 2H), 3.44-3.53 (m, 1H), 3.24-3.30 (m, 2H), 3.12-3.23 (m, 1H), 3.02-3.10 (m, 1H), 0.97 (s, 9H) | 475 |
| 195 | 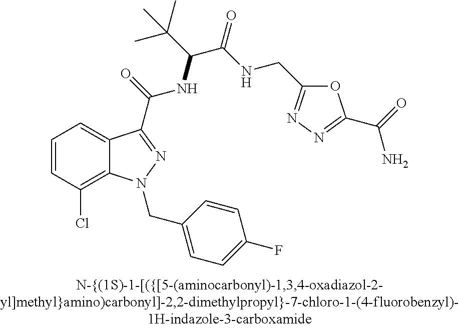 | ¹H NMR (400 MHz, DMSO-d6) δ ppm 9.16 (t, J = 5.49 Hz, 1H), 8.58 (br. s., 1H), 8.14-8.25 (m, 2H), 7.68 (d, J = 9.52 Hz, 1H), 7.54 (d, J = 7.32 Hz, 1H), 7.29 (t, J = 7.87 Hz, 1H), 7.08-7.18 (m, 4H), 6.03 (d, J = 4.39 Hz, 2H), 4.53-4.71 (m, 2H), 0.98 (s, 9H). | 542 |
| 196 | 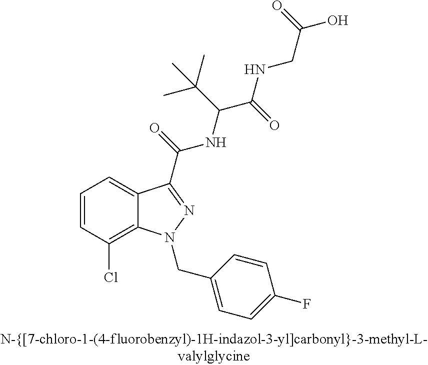 | ¹H NMR (400 MHz, DMSO-d6) δ ppm 8.64 (t, J = 5.86 Hz, 1H), 8.17-8.24 (m, 1H), 7.62-7.71 (m, 1H), 7.48-7.57 (m, 1H), 7.26-7.33 (m, 1H), 7.09-7.18 (m, 4H), 5.98 (s, 2H), 4.56 (d, J = 9.88 Hz, 1H), 3.67-3.88 (m, 2H), 0.99 (s, 9H) | 475 |
| 197 | 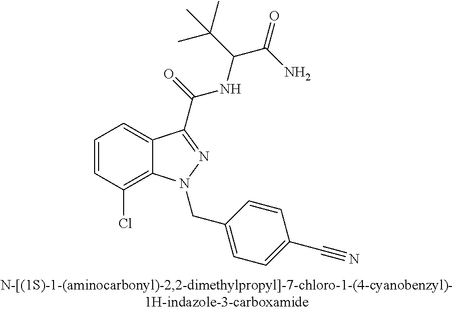 | ¹H NMR (400 MHz, DMSO-d6) δ ppm 8.23 (d, J = 8.05 Hz, 1H), 7.79 (d, J = 8.05 Hz, 2H), 7.73 (br. s., 1H), 7.65 (d, J = 9.52 Hz, 1H), 7.55 (d, J = 7.69 Hz, 1H), 7.26-7.34 (m, 2H), 7.20 (d, J = 8.42 Hz, 2H), 6.12-6.19 (m, 2H), 4.46 (d, J = 9.52 Hz, 1H), 0.97 (s, 9H) | 424 |
| 198 | 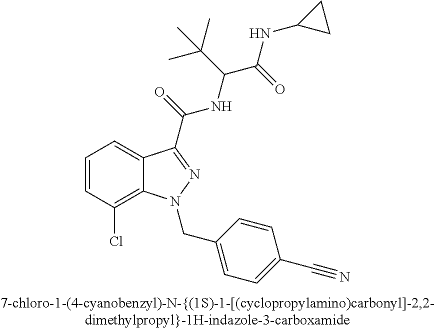 | ¹H NMR (400 MHz, DMSO-d6) δ ppm 8.33-8.39 (m, 1H), 8.22 (d, J = 8.05 Hz, 1H), 7.79 (d, J = 8.42 Hz, 2H), 7.62 (d, J = 9.52 Hz, 1H), 7.55 (d, J = 7.32 Hz, 1H), 7.30 (t, J = 7.69 Hz, 1H), 7.20 (d, J = 8.05 Hz, 2H), 6.13-6.20 (m, 2H), 4.40 (d, J = 9.52 Hz, 1H), 2.59-2.71 (m, 1H), 0.94 (s, 9H), 0.57-0.66 (m, 2H), 0.34-0.46 (m, 2H) | 464 |
| 199 | 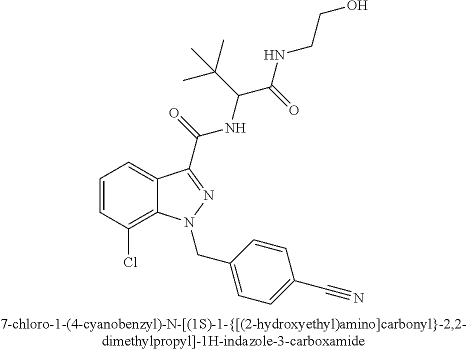 | ¹H NMR (400 MHz, DMSO-d6) δ ppm 8.31 (t, J = 5.49 Hz, 1H), 8.22 (d, J = 8.05 Hz, 1H), 7.79 (d, J = 8.05 Hz, 2H), 7.66 (d, J = 9.88 Hz, 1H), 7.55 (d, J = 7.32 Hz, 1H), 7.30 (t, J = 7.87 Hz, 1H), 7.20 (d, J = 8.42 Hz, 2H), 6.10-6.23 (m, 2H), 4.67 (t, J = 5.31 Hz, 1H), 4.50 (d, J = 9.52 Hz, 1H), 3.40 (q, J = 5.61 Hz, 2H), 3.05-3.25 (m, 2H), 0.96 (s, 9H) | 468 |
| 200 | 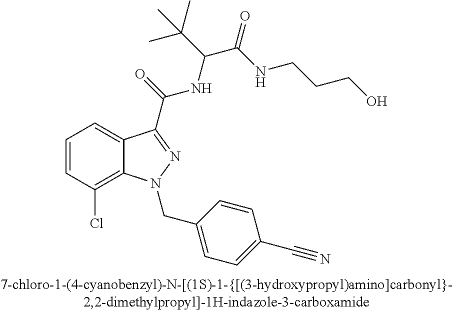 | ¹H NMR (400 MHz, DMSO-d6) δ ppm 8.28 (t, J = 5.31 Hz, 1H), 8.22 (d, J = 8.42 Hz, 1H), 7.79 (d, J = 8.05 Hz, 2H), 7.65 (d, J = 9.88 Hz, 1H), 7.55 (d, J = 7.69 Hz, 1H), 7.30 (t, J = 7.87 Hz, 1H), 7.20 (d, J = 8.05 Hz, 2H), 6.10-6.22 (m, 2H), 4.39-4.51 (m, 2H), 3.39 (q, J = 6.10 Hz, 2H), 2.97-3.25 (m, 2H), 1.48-1.61 (m, 2H), 0.96 (s, 9H) | 482 |
| 201 | 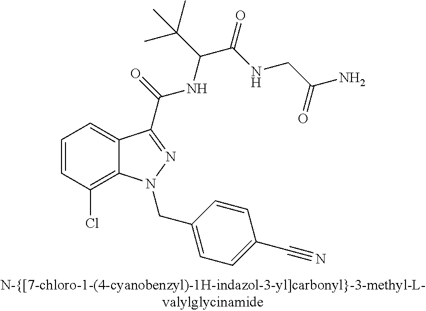 | ¹H NMR (400 MHz, DMSO-d6) δ ppm 8.49 (t, J = 5.67 Hz, 1H), 8.22 (d, J = 8.05 Hz, 1H), 7.79 (d, J = 8.05 Hz, 2H), 7.69 (d, J = 9.52 Hz, 1H), 7.55 (d, J = 7.32 Hz, 1H), 7.26-7.36 (m, 2H), 7.20 (d, J = 8.05 Hz, 2H), 7.00 (br. s., 1H), 6.08-6.23 (m, 2H), 4.54 (d, J = 9.52 Hz, 1H), 3.64-3.72 (m, 2H), 0.98 (s, 9H) | 481 |
| 202 | 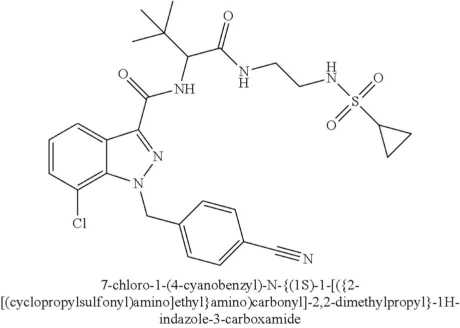 | ¹H NMR (400 MHz, DMSO-d6) δ ppm 8.41 (t, J = 5.49 Hz, 1H), 8.22 (d, J = 8.05 Hz, 1H), 7.79 (d, J = 8.05 Hz, 2H), 7.66 (d, J = 9.52 Hz, 1H), 7.55 (d, J = 7.32 Hz, 1H), 7.31 (t, J = 7.69 Hz, 1H), 7.20 (d, J = 8.05 Hz, 2H), 7.11 (t, J = 5.86 Hz, 1H), 6.09-6.23 (m, 2H), 4.46 (d, J = 9.52 Hz, 1H), 3.12-3.27 (m, 2H), 3.03 (t, J = 5.86 Hz, 2H), 0.96 (s, 9H), 0.81-0.92 (m, 4H) | 571 |
| 203 | 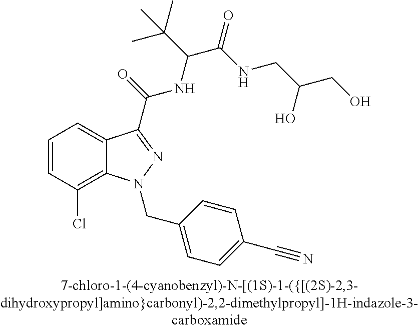 | ¹H NMR (400 MHz, DMSO-d6) δ ppm 8.27 (t, J = 5.49 Hz, 1H), 8.22 (d, J = 8.42 Hz, 1H), 7.79 (d, J = 8.05 Hz, 2H), 7.67 (d, J = 9.52 Hz, 1H), 7.55 (d, J = 7.32 Hz, 1H), 7.30 (t, J = 7.87 Hz, 1H), 7.20 (d, J = 8.05 Hz, 2H), 6.08-6.23 (m, 2H), 4.72 (d, J = 5.12 Hz, 1H), 4.50-4.59 (m, 2H), 3.44-3.55 (m, 1H), 3.23-3.29 (m, 3H), 2.88-3.00 (m, 1H), 0.96 (s, 9H) | 498 |
| 204 | 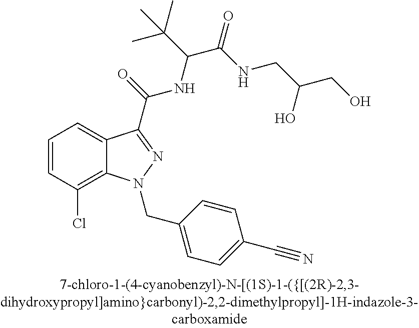 | ¹H NMR (400 MHz, DMSO-d6) δ ppm 8.28 (t, J = 5.49 Hz, 1H), 8.22 (d, J = 8.05 Hz, 1H), 7.79 (d, J = 8.42 Hz, 2H), 7.67 (d, J = 9.52 Hz, 1H), 7.55 (d, J = 7.32 Hz, 1H), 7.30 (t, J = 7.87 Hz, 1H), 7.20 (d, J = 8.05 Hz, 2H), 6.08-6.23 (m, 2H), 4.74 (d, J = 4.76 Hz, 1H), 4.49-4.59 (m, 2H), 3.43-3.52 (m, 1H), 3.28 (t, J = 5.49 Hz, 2H), 3.00-3.22 (m, 2H), 0.96 (s, 9H) | 498 |
| 205 | 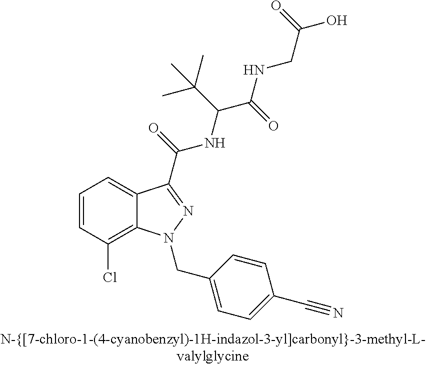 | ¹H NMR (400 MHz, DMSO-d6) δ ppm 8.66 (t, J = 5.86 Hz, 1H), 8.22 (d, J = 8.05 Hz, 1H), 7.79 (d, J = 8.42 Hz, 2H), 7.67 (d, J = 9.52 Hz, 1H), 7.55 (d, J = 7.69 Hz, 1H), 7.31 (t, J = 7.87 Hz, 1H), 7.17-7.22 (m, 2H), 6.10-6.23 (m, 2H), 4.56 (d, J = 9.88 Hz, 1H), 3.69-3.88 (m, 2H), 0.99 (s, 9H) | 482 |
| 206 | 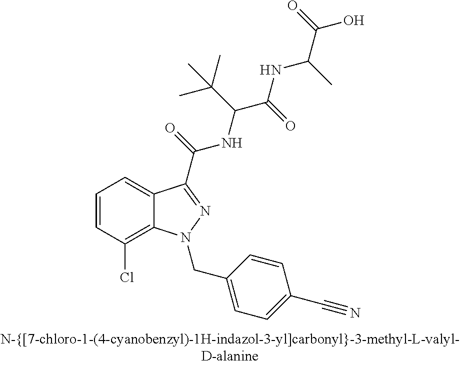 | ¹H NMR (400 MHz, DMSO-d6) δ ppm 8.67 (d, J = 7.32 Hz, 1H), 8.23 (d, J = 8.05 Hz, 1H), 7.79 (d, J = 8.05 Hz, 2H), 7.66 (d, J = 9.88 Hz, 1H), 7.55 (d, J = 7.69 Hz, 1H), 7.31 (t, J = 7.87 Hz, 1H), 7.20 (d, J = 8.05 Hz, 2H), 6.09-6.23 (m, 2H), 4.61 (d, J = 9.88 Hz, 1H), 4.26 (t, J = 7.32 Hz, 1H), 1.26 (d, J = 7.32 Hz, 3H), 0.96 (s, 9H) | 496 |
| 207 | 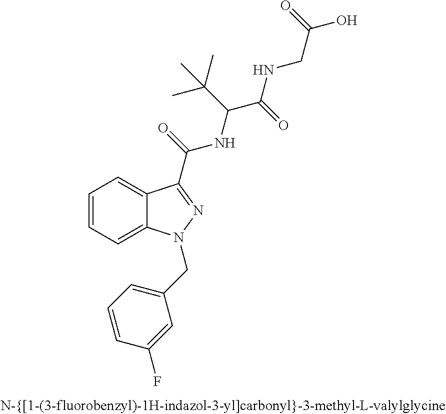 | ¹H NMR (400 MHz, DMSO-d₆) δ 1.00 (s, 9H), 3.74 (dd, J = 6, 17 Hz, 1H), 3.85 (dd, J = 6, 17 Hz, 1H), 4.56 (d, J = 10 Hz, 1H), 5.82 (s, 2H), 7.03 (d, J = 8 Hz, 1H), 7.12 (m, 2H), 7.30 (t, J = 7 Hz, 1H), 7.34-7.40 (m, 1H), 7.47 (t, J = 8 Hz, 1H), 7.63 (d, J = 10 Hz, 1H), 7.79 (d, J = 8 Hz, 1H), 8.18 (d, J = 8 Hz, 1H), 8.64 (t, J = 5 Hz, 1H) | 441 |
| 208 | 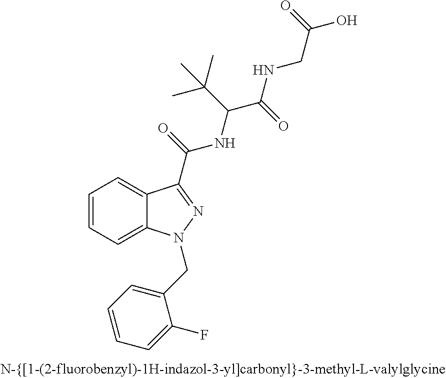 | ¹H NMR (400 MHz, DMSO-d₆) δ 0.99 (s, 9H), 1.23 (s, 1H), 3.73 (dd, J = 6, 18 Hz, 1H), 3.84 (dd, J = 6, 18 Hz, 1H), 4.55 (d, J = 10 Hz, 1H), 5.84 (s, 2H), 7.15 (m, 2H), 7.23 (t, J = 9 Hz, 1H), 7.30 (t, J = 7 Hz, 1H), 7.34 (m, 1H), 7.48 (t, J = 8 Hz, 1H), 7.58 (d, J = 10 Hz, 1H), 7.78 (d, J = 8 Hz, 1H), 8.18 (d, J = 8 Hz, 1H), 8.63 (m, 1H), 12.54 (s, 1H) | 441 |
| 209 | 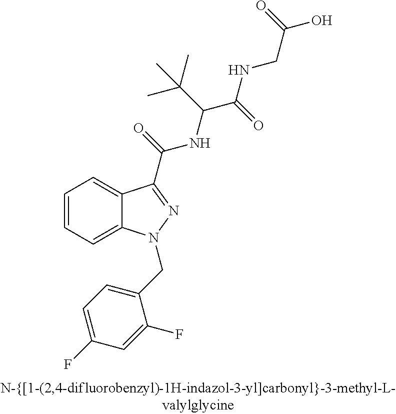 | ¹H NMR (400 MHz, DMSO-d₆) δ 0.99 (s, 9H), 1.23 (s, 1H), 1.90 (s, 1H), 3.70-3.87 (m, 2H), 4.55 (d, J = 10 Hz, 1H), 7.03-7.08 (m, 1H), 7.24-7.32 (m, 3H), 7.48 (t, J = 8 Hz, 1H), 7.57 (d, J = 10 Hz, 1H), 7.79 (d, J = 9 Hz, 1H), 8.17 (d, J = 8 Hz, 1H), 8.64 (t, J = 6 Hz, 1H) | 459 |
| 210 | 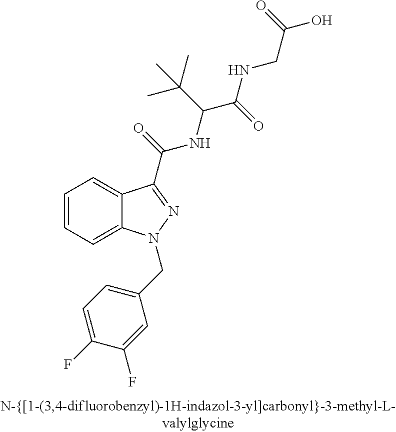 | ¹H NMR (400 MHz, DMSO-d₆) δ 1.00 (s, 9H), 3.64-3.77 (m, 2H), 4.56 (d, J = 10 Hz, 1H), 5.78 (d, J = 14 Hz, 2H), 7.3 (t, J = 7 Hz, 1H), 7.36-7.49 (m, 3H), 7.63 (d, J = 10 Hz, 1H), 7.81 (d, J = 8 Hz, 1H), 8.18 (d, J = 8 Hz, 1H), 8.49 (s, 1H) | 459 |
| 211 | 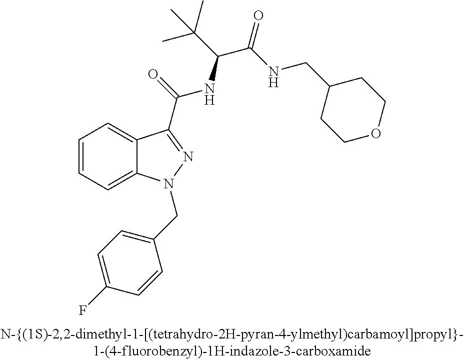 | ¹H NMR (400 MHz, DMSO-d6) δ ppm 0.96 (s, 9H) 1.14 (qd, J = 12.20, 4.39 Hz, 2H) 1.54 (d, J = 13.18 Hz, 2H) 1.61 (td, J = 10.98, 4.39 Hz, 1H) 2.88 (ddd, J = 12.81, 6.22, 5.86 Hz, 1H) 3.07 (ddd, J = 13.00, 6.41, 6.22 Hz, 1H) 3.17-3.27 (m, 2H) 3.33 (s, 3H) 3.80 (dd, J = 11.35, 2.56 Hz, 2H) 4.50 (d, J = 9.52 Hz, 1H) 5.77 (s, 2H) 7.15 (t, J = 8.79 Hz, 2H) 7.30 (dt, J = 8.79, 4.39 Hz, 2H) 7.45 (t, J = 7.69 Hz, 1H) 7.60 (d, J = 9.52 Hz, 1H) 7.79 (d, J = 8.79 Hz, 1H) 8.16 (d, J = 8.05 Hz, 1H) 8.32 (t, J = 5.49 Hz, 1H) | 481 |
| 212 | 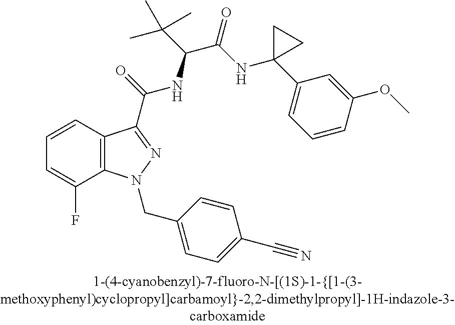 | ¹H NMR (400 MHz, DMSO-d6) δ ppm 9.03 (s, 1H) 8.02 (d, J = 7.32 Hz, 1H) 7.80 (d, J = 8.05 Hz, 2H) 7.63 (d, J = 9.88 Hz, 1H) 7.25-7.35 (m, 4H) 7.13 (t, J = 8.05 Hz, 1H) 6.64-6.80 (m, 3H) 5.93 (s, 2H) 4.48 (d, J = 9.52 Hz, 1H) 3.62 (s, 3H) 1.20-1.31 (m, 1H) 1.05-1.17 (m, 3H) 0.96 (s, 9H). | 554 |
| 213 | 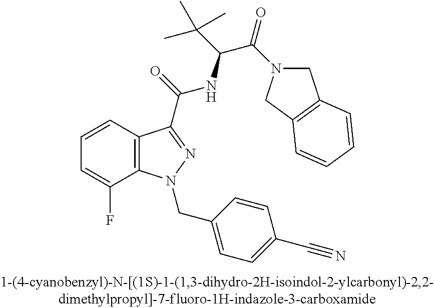 | ¹H NMR (400 MHz, DMSO-d6) δ ppm 7.99 (d, J = 7.32 Hz, 1H) 7.80 (d, J = 8.42 Hz, 2H) 7.67 (d, J = 9.15 Hz, 1H) 7.23-7.46 (m, 7H) 5.94 (s, 2H) 5.10 (s, 2H) 4.89 (d, J = 9.52 Hz, 1H) 4.61-4.80 (m, 2H) 1.07 (s, 9H). | 510 |
| 214 | 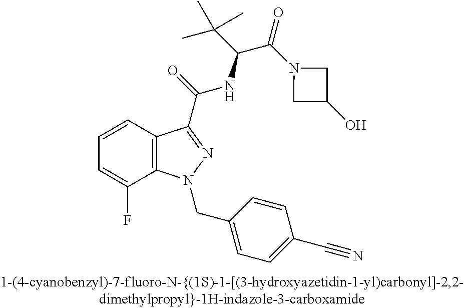 | ¹H NMR (400 MHz, DMSO-d6) δ ppm 8.00 (dd, J = 7.32, 4.03 Hz, 1H) 7.81 (d, J = 8.05 Hz, 2H) 7.59 (dd, J = 9.34, 4.58 Hz, 1H) 7.25-7.39 (m, 4H) 5.93 (br. s., 2H) 5.81 (d, J = 5.49 Hz, 1H) 5.74 (d, J = 5.86 Hz, 1H) 4.42-4.53 (m, 3H) 3.95-4.17 (m, 2H) 3.61 (td, J = 9.88, 2.20 Hz, 1H) 0.99 (d, J = 3.66 Hz, 9H). | 464 |
| 215 | 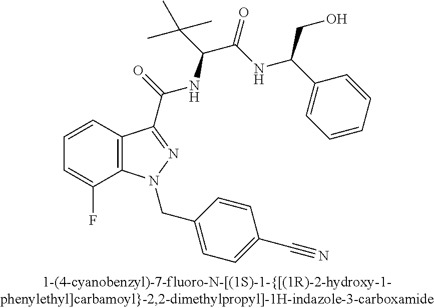 | ¹H NMR (400 MHz, DMSO-d6) δ ppm 8.83 (d, J = 8.05 Hz, 1H) 8.04 (d, J = 7.32 Hz, 1H) 7.81 (d, J = 8.42 Hz, 2H) 7.64 (d, J = 9.88 Hz, 1H) 7.18-7.43 (m, 8H) 5.94 (s, 2H) 4.83-4.93 (m, 2H) 4.64 (d, J = 9.52 Hz, 1H) 3.47-3.60 (m, 2H) 0.88 (s, 9H). | 528 |
| 216 | 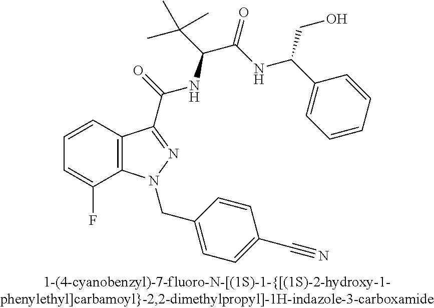 | ¹H NMR (400 MHz, DMSO-d6) δ ppm 8.65 (d, J = 8.05 Hz, 1H) 8.00 (d, J = 7.32 Hz, 1H) 7.78 (d, J = 8.42 Hz, 2H) 7.62 (d, J = 9.52 Hz, 1H) 7.17-7.38 (m, 8H) 5.91 (s, 2H) 4.83-4.95 (m, 2H) 4.62 (d, J = 9.52 Hz, 1H) 3.55 (t, J = 5.67 Hz, 2H) 1.02 (s, 9H). | 528 |
| 217 | 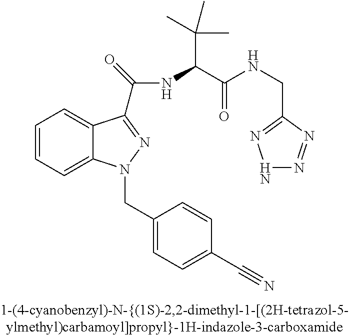 | ¹H NMR (400 MHz, DMSO-d₆) δ ppm 0.95 (br. s., 9H) 4.52-4.63 (m, 3H) 5.92 (br. s., 2H) 7.28-7.39 (m, 3H) 7.48 (d, J = 6.96 Hz, 1H) 7.62 (d, J = 9.15 Hz, 1H) 7.75-7.85 (m, 3H) 8.18 (d, J = 8.05 Hz, 1H) 9.04 (br. s., 1H) | 472 |
| 218 | 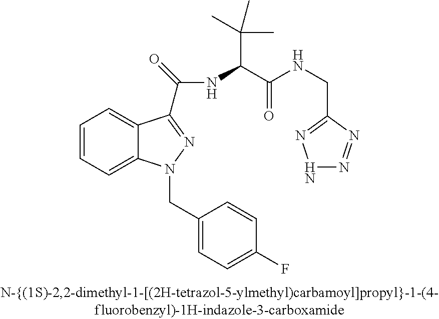 | ¹H NMR (400 MHz, DMSO-d₆) δ ppm 0.96 (s, 9H) 4.54 (d, J = 9.88 Hz, 1H) 4.59 (d, J = 5.86 Hz, 2H) 5.78 (s, 2H) 7.16 (t, J = 8.79 Hz, 2H) 7.26-7.36 (m, 3H) 7.46 (t, J = 7.51 Hz, 1H) 7.63 (d, J = 9.52 Hz, 1H) 7.80 (d, J = 8.42 Hz, 1H) 8.17 (d, J = 8.05 Hz, 1H) 9.05 (t, J = 5.49 Hz, 1H) | 465 |
| 219 | 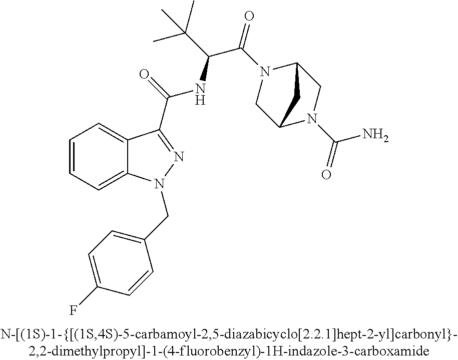 | ¹H NMR (400 MHz, DMSO-d6) δ ppm 1.00 (d, J = 10.25 Hz, 9H) 1.69-1.80 (m, 1H) 1.80-1.93 (m, 1H) 3.17 (dd, J = 23.43, 9.52 Hz, 1H) 3.24-3.31 (m, 2H) 3.58-3.70 (m, 1H) 4.47 (d, J = 31.48 Hz, 1H) 4.86, 4.56 (dd, J = 120, 8 Hz, 1H) 4.94 (d, J = 92 Hz, 1H) 5.70-5.83 (m, 2H) 5.92 (d, J = 20.50 Hz, 2H) 7.15 (t, J = 8.79 Hz, 2H) 7.24-7.36 (m, 3H) 7.45 (t, 1H) 7.55 (dd, J = 30.75, 9.52 Hz, 1H) 7.79 (t, J = 8.42 Hz, 1H) 8.15 (t, J = 8.42 Hz, 1H) | 507 |
| 220 | 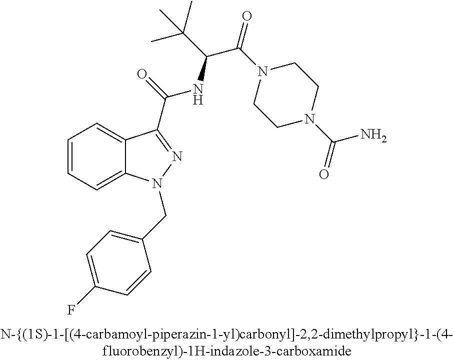 | ¹H NMR (400 MHz, DMSO-d6) δ ppm 1.01 (s, 9H) 3.18-3.35 (m, 3H) 3.35-3.49 (m, 2H) 3.49-3.58 (m, 1H) 3.58-3.75 (m, 2H) 5.06 (d, J = 9.52 Hz, 1H) 5.77 (s, 2H) 6.01 (s, 2H) 7.15 (t, J = 8.79 Hz, 2H) 7.25-7.36 (m, 3H) 7.46 (t, J = 7.69 Hz, 1H) 7.64 (d, J = 9.52 Hz, 1H) 7.78 (d, J = 8.79 Hz, 1H) 8.17 (d, J = 8.05 Hz, 1H) | 495 |
| 221 | 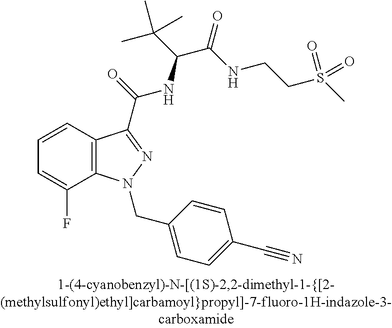 | ¹H NMR (400 MHz, DMSO-d6) δ ppm 8.55 (t, J = 5.49 Hz, 1H) 7.98-8.04 (m, 1H) 7.80 (d, J = 8.05 Hz, 2H) 7.64 (d, J = 9.52 Hz, 1H) 7.21-7.37 (m, 4H) 5.93 (s, 2H) 4.47 (d, J = 9.88 Hz, 1H) 3.41-3.59 (m, 2H) 2.99 (s, 3H) 0.98 (s, 9H). | 514 |
| 222 | 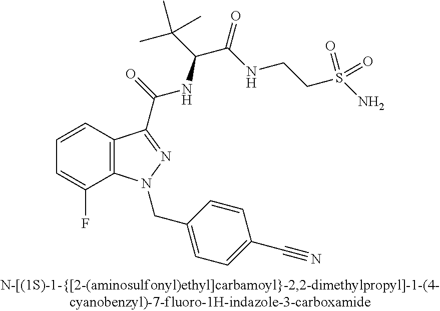 | ¹H NMR (400 MHz, DMSO-d6) δ ppm 8.46 (t, J = 5.67 Hz, 1H) 7.98-8.06 (m, 1H) 7.80 (d, J = 8.05 Hz, 2H) 7.64 (d, J = 9.88 Hz, 1H) 7.23-7.37 (m, 4H) 6.87 (s, 2H) 5.93 (s, 2H) 4.45 (d, J = 9.88 Hz, 1H) 3.38-3.59 (m, 2H) 3.06-3.22 (m, 2H) 0.98 (s, 9H). | 515 |
| 223 | 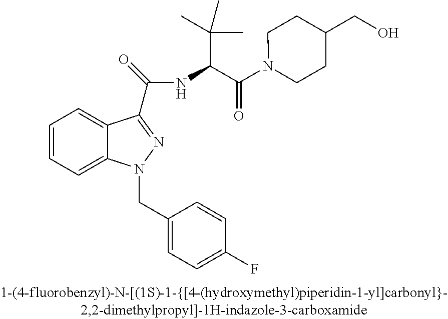 | ¹H NMR (400 MHz, DMSO-d₆) δ ppm 0.99 (d, J = 9.88 Hz, 9H) 1.63 (br. s., 2H) 1.76 (br. s., 2H) 2.59 (d, J = 12.81 Hz, 2H) 3.08 (br. s., 2H) 3.20 (t, J = 5.49 Hz, 1H) 4.20 (d, J = 12.81 Hz, 1H) 4.39-4.48 (m, 2H) 5.09 (dd, J = 9.34, 7.14 Hz, 1H) 5.77 (s, 2H) 7.15 (t, J = 8.24 Hz, 2H) 7.26-7.36 (m, 3H) 7.45 (t, J = 7.69 Hz, 1H) 7.57-7.66 (m, 1H) 7.78 (d, J = 8.42 Hz, 1H) 8.17 (d, J = 8.42 Hz, 1H) | 481 |
| 224 | 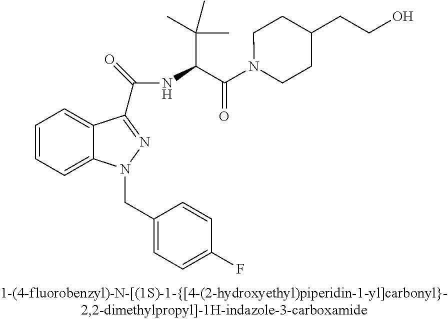 | ¹H NMR (400 MHz, DMSO-d₆) δ ppm 0.99 (d, J = 10.98 Hz, 9H) 1.30 (q, J = 6.47 Hz, 1H) 1.38 (q, J = 6.59 Hz, 1H) 1.63 (br. s., 2H) 1.75 (br. s., 2H) 2.59 (d, J = 12.45 Hz, 2H) 3.08 (d, J = 6.96 Hz, 1H) 3.38-3.48 (m, 2H) 4.17 (d, J = 13.91 Hz, 1H) 4.27-4.35 (m, 1H) 4.36-4.45 (m, 1H) 5.08 (dd, J = 9.34, 6.77 Hz, 1H) 5.77 (s, 2H) 7.12-7.19 (m, 2H) 7.26-7.35 (m, 3H) 7.46 (t, J = 7.69 Hz, 1H) 7.61 (dd, J = 14.46, 9.70 Hz, 1H) 7.78 (dd, J = 8.60, 3.11 Hz, 1H) 8.17 (d, J = 8.05 Hz, 1H) | 495 |
| 225 | 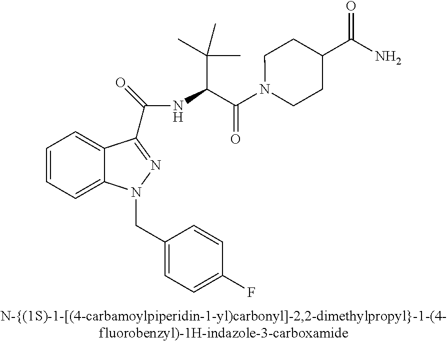 | ¹H NMR (400 MHz, DMSO-d₆) δ ppm 1.00 (d, J = 10.98 Hz, 9H) 1.36 (d, J = 12.81 Hz, 1H) 1.55 (br. s., 1H) 1.78 (br. s., 2H) 2.35 (dd, J = 7.87, 3.48 Hz, 1H) 2.67 (t, J = 11.53 Hz, 1H) 3.14 (d, J = 12.81 Hz, 1H) 4.21 (br. s., 1H) 4.33-4.45 (m, 1H) 5.08 (dd, J = 9.15, 7.69 Hz, 1H) 5.77 (s, 2H) 6.65-6.81 (m, 1H) 7.15 (t, J = 8.60 Hz, 2H) 7.26-7.35 (m, 4H) 7.45 (t, J = 7.69 Hz, 1H) 7.57-7.67 (m, 1H) 7.77 (dd, J = 8.42, 4.03 Hz, 1H) 8.17 (dd, J = 8.05, 5.13 Hz, 1H) | 494 |
| 226 | 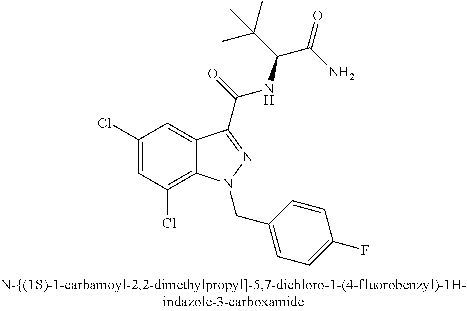 | ¹H NMR (400 MHz, DMSO-d6) δ ppm 8.15-8.23 (m, 1H), 7.67-7.74 (m, 2H), 7.60-7.67 (m, 1H), 7.21-7.27 (m, 1H), 7.11-7.19 (m, 4H), 5.99-6.05 (m, 2H), 4.46 (d, J = 9.88 Hz, 1H), 0.98 (s, 9H) | 451 |
| 227 | 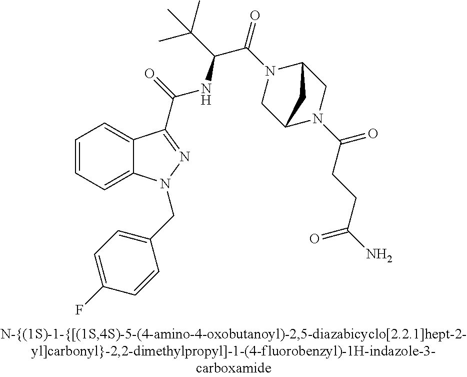 | ¹H NMR (400 MHz, DMSO-d6) δ ppm 0.94-1.07 (m, 9H) 1.70-2.00 (m, 2H) 2.20-2.34 (m, 3H) 3.58 (q, J = 10.25 Hz, 1H) 4.51-4.61 (m, 1H) 4.63-4.94 (m, 4H) 4.99-5.11 (m, 1H) 5.76 (s, 2H) 6.62 (br. s., 1H) 7.15 (t, J = 8.79 Hz, 3H) 7.25-7.37 (m, 3H) 7.41-7.53 (m, 2H) 7.56-7.64 (m, 1H) 7.78 (t, J = 7.32 Hz, 1H) 8.16 (t, J = 7.69 Hz, 1H) | 563 |
| 228 | 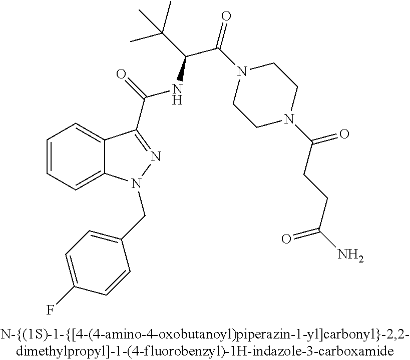 | ¹H NMR (400 MHz, DMSO-d6) δ ppm 1.01 (s, 9H) 2.30 (t, J = 6.96 Hz, 2H) 3.37-3.82 (m, 10H) 5.06 (d, J = 8.79 Hz, 1H) 5.77 (d, J = 1.46 Hz, 2H) 6.66 (br. s., 1H) 7.15 (t, J = 9.15 Hz, 2H) 7.23 (br. s., 1H) 7.26-7.36 (m, 3H) 7.42-7.49 (m, 1H) 7.64 (d, J = 9.52 Hz, 1H) 7.78 (d, J = 8.79 Hz, 1H) 8.17 (d, J = 8.05 Hz, 1H) | 551 |
| 229 | 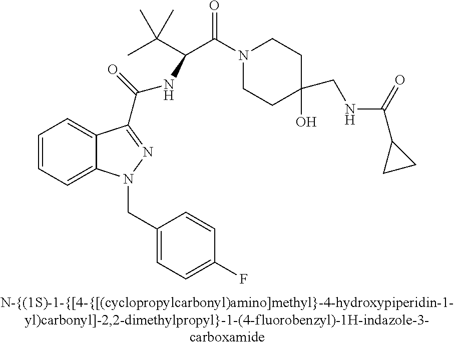 | ¹H NMR (400 MHz, DMSO-d₆) δ ppm 0.46-0.70 (m, 4H) 0.99 (d, J = 6.22 Hz, 9H) 1.29-1.54 (m, 4H) 1.54-1.70 (m, 1H) 2.94-3.18 (m, 3H) 3.34-3.49 (m, 1H) 3.85-4.06 (m, 1H) 4.15 (d, J = 12.81 Hz, 1H) 4.64 (d, J = 7.69 Hz, 1H) 5.09 (t, J = 10.62 Hz, 1H) 5.77 (s, 2H) 7.15 (t, J = 8.79 Hz, 2H) 7.26-7.36 (m, 3H) 7.46 (t, J = 7.69 Hz, 1H) 7.61 (t, J = 9.34 Hz, 1H) 7.78 (d, J = 8.42 Hz, 1H) 7.90-8.01 (m, 1H) 8.17 (d, J = 8.05 Hz, 1H) | 564 |
| 230 | 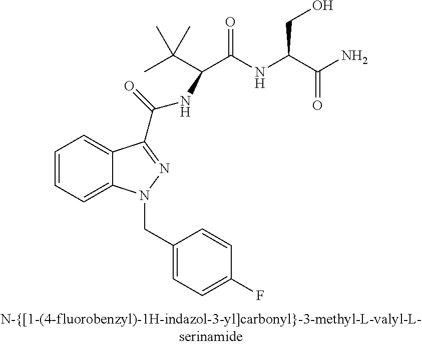 | ¹H NMR (400 MHz, DMSO-d₆) δ ppm 0.99 (s, 9H) 3.52-3.61 (m, 2H) 4.29 (d, J = 7.69 Hz, 1H) 4.60 (d, J = 9.52 Hz, 1H) 4.82 (t, J = 5.49 Hz, 1H) 5.77 (s, 2H) 7.00 (br. s., 1H) 7.15 (t, J = 8.79 Hz, 2H) 7.22 (br. s., 1H) 7.28-7.36 (m, 3H) 7.45 (t, J = 7.69 Hz, 1H) 7.64 (d, J = 9.52 Hz, 1H) 7.78 (d, J = 8.42 Hz, 1H) 8.16-8.25 (m, 2H) | 470 |
| 231 | 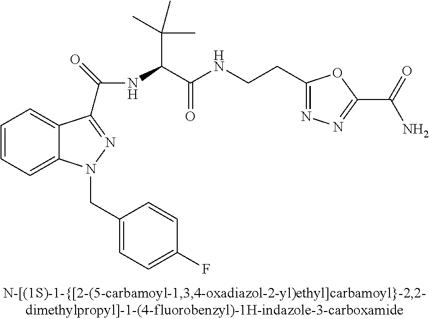 | ¹H NMR (400 MHz, DMSO-d₆) δ ppm 0.89-0.99 (m, 9H) 3.07-3.15 (m, 2H) 3.38-3.48 (m, 1H) 3.57-3.68 (m, 1H) 4.42 (d, J = 9.88 Hz, 1H) 5.77 (s, 2H) 7.15 (t, J = 8.79 Hz, 2H) 7.26-7.35 (m, 3H) 7.45 (t, J = 7.69 Hz, 1H) 7.57 (d, J = 9.52 Hz, 1H) 7.77 (d, J = 8.79 Hz, 1H) 8.12 (s, 1H) 8.16 (d, J = 8.42 Hz, 1H) 8.45-8.54 (m, 2H) | 522 |
| 232 | 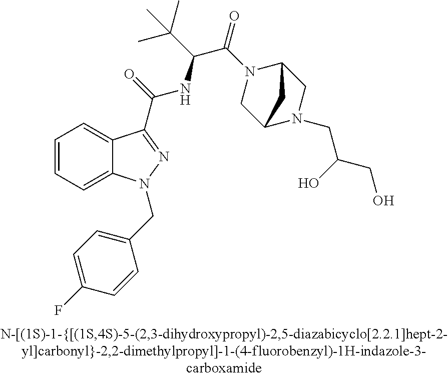 | ¹H NMR (400 MHz, DMSO-d6) δ ppm 1.03 (d, J = 11.72 Hz, 9H) 1.70 (t, J = 8.42 Hz, 1H) 1.86, 1.54 (dd, J = 128, 8 Hz, 1H) 2.40-2.50 (m, 2H) 2.64 (dd, J = 12.45, 5.86 Hz, 1H) 2.89-3.00 (m, 1H) 3.14 (d, J = 10.25 Hz, 1H) 3.30-3.38 (m, 2H) 3.39-3.54 (m, 1H) 3.63 (d, J = 28.56 Hz, 1H) 3.76 (d, J = 9.52 Hz, 1H) 4.39 (d, J = 20.50 Hz, 1H) 4.70 (d, J = 56 Hz, 1H) 4.82, 4.60 (dd, J = 88, 8 Hz, 1H) 5.77 (s, 2H) 7.16 (t, J = 8.79 Hz, 2H) 7.25-7.37 (m, 3H) 7.42-7.53 (m, 2H) 7.58 (d, J = 9.52 Hz, 1H) 7.78 (d, J = 8.79 Hz, 1H) 8.16 (t, J = 7.69 Hz, 1H) | 538 |
| 233 | 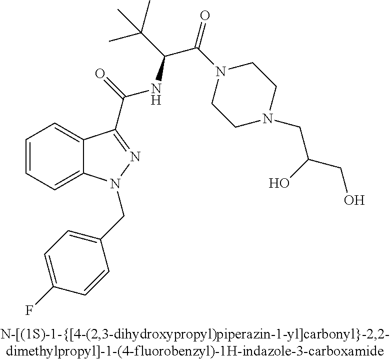 | ¹H NMR (400 MHz, DMSO-d6) δ ppm 1.00 (s, 9H) 2.19-2.33 (m, 2H) 2.33-2.46 (m, 4H) 3.32 (d, J = 5.13 Hz, 2H) 3.52 (br. s., 2H) 3.57-3.74 (m, 3H) 3.96-4.19 (m, 1H) 4.34-4.47 (m, 1H) 5.06 (d, J = 9.52 Hz, 1H) 5.77 (s, 2H) 7.15 (t, J = 8.79 Hz, 2H) 7.26-7.37 (m, 3H) 7.46 (t, J = 7.69 Hz, 1H) 7.62 (d, J = 9.52 Hz, 1H) 7.78 (d, J = 8.79 Hz, 1H) 8.17 (d, J = 8.05 Hz, 1H) | 526 |
| 234 | 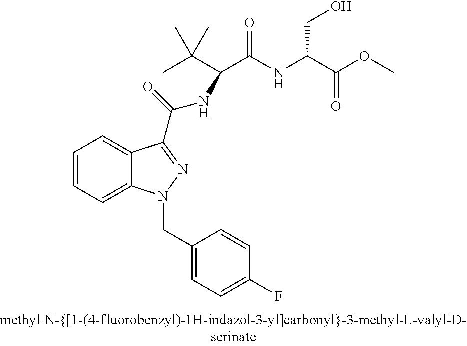 | ¹H NMR (400 MHz, DMSO-d₆) δ ppm 0.98 (s, 9H) 3.60 (s, 3H) 3.61-3.71 (m, 2H) 4.41 (d, J = 7.69 Hz, 1H) 4.68 (d, J = 9.88 Hz, 1H) 4.98 (t, J = 5.49 Hz, 1H) 5.77 (s, 2H) 7.15 (t, J = 8.97 Hz, 2H) 7.27-7.35 (m, 3H) 7.46 (t, J = 7.69 Hz, 1H) 7.63 (d, J = 9.88 Hz, 1H) 7.77 (d, J = 8.79 Hz, 1H) 8.18 (d, J = 8.05 Hz, 1H) 8.65 (d, J = 7.69 Hz, 1H) | 485 |
| 235 | 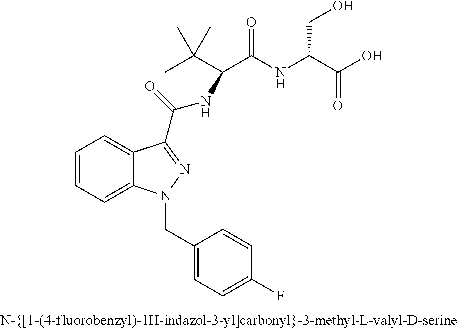 | ¹H NMR (400 MHz, DMSO-d₆) δ ppm 0.93-1.02 (m, 9H) 3.60-3.70 (m, 2H) 4.27-4.37 (m, 1H) 4.69 (d, J = 9.88 Hz, 1H) 5.77 (s, 2H) 7.15 (t, J = 8.97 Hz, 2H) 7.28-7.35 (m, 3H) 7.45 (t, J = 7.69 Hz, 1H) 7.64 (d, J = 9.88 Hz, 1H) 7.77 (d, J = 8.42 Hz, 1H) 8.18 (d, J = 8.05 Hz, 1H) 8.50 (d, J = 8.05 Hz, 1H) | 471 |
| 236 | 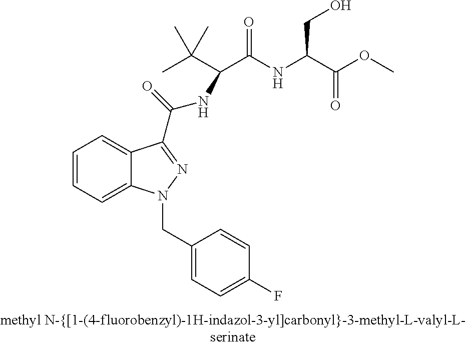 | ¹H NMR (400 MHz, DMSO-d₆) δ ppm 1.01 (s, 9H) 3.63 (s, 3H) 3.68 (dt, J = 10.43, 5.40 Hz, 2H) 4.38 (d, J = 6.59 Hz, 1H) 4.66 (d, J = 9.88 Hz, 1H) 4.98 (t, J = 5.67 Hz, 1H) 5.77 (s, 2H) 7.15 (t, J = 8.97 Hz, 2H) 7.26-7.35 (m, 3H) 7.46 (t, J = 7.69 Hz, 1H) 7.60 (d, J = 9.52 Hz, 1H) 7.78 (d, J = 8.42 Hz, 1H) 8.17 (d, J = 8.42 Hz, 1H) 8.59 (d, J = 7.32 Hz, 1H) | 485 |
| 237 | 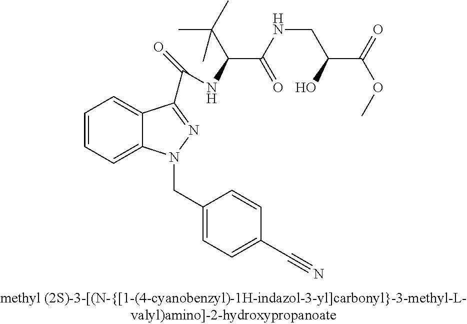 | ¹H NMR (400 MHz, DMSO-d₆) δ ppm 0.91-1.00 (m, 9H) 3.17-3.24 (m, 1H) 3.42-3.51 (m, 1H) 3.57 (s, 3H) 4.13 (d, J = 5.86 Hz, 1H) 4.52 (d, J = 9.52 Hz, 1H) 5.61 (d, J = 5.86 Hz, 1H) 5.91 (s, 2H) 7.28-7.38 (m, 3H) 7.46 (t, J = 7.69 Hz, 1H) 7.60 (d, J = 9.88 Hz, 1H) 7.76 (d, J = 8.42 Hz, 1H) 7.79 (d, J = 8.05 Hz, 2H) 8.19 (d, J = 8.05 Hz, 1H) 8.36 (t, J = 5.67 Hz, 1H) | 492 |
| 238 | 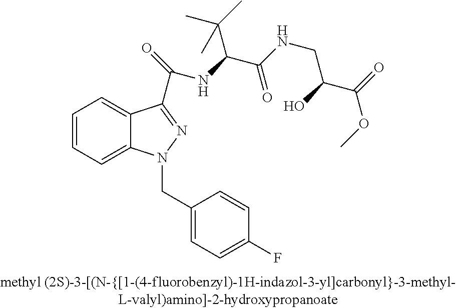 | ¹H NMR (400 MHz, DMSO-d₆) δ ppm 0.97 (s, 9H) 3.17-3.24 (m, 1H) 3.47 (d, J = 8.05 Hz, 1H) 3.57 (s, 3H) 4.13 (d, J = 5.86 Hz, 1H) 4.52 (d, J = 9.52 Hz, 1H) 5.61 (d, J = 5.86 Hz, 1H) 5.77 (s, 2H) 7.15 (t, J = 8.97 Hz, 2H) 7.26-7.35 (m, 3H) 7.45 (t, J = 7.69 Hz, 1H) 7.60 (d, J = 9.52 Hz, 1H) 7.78 (d, J = 8.79 Hz, 1H) 8.17 (d, J = 8.05 Hz, 1H) 8.36 (t, J = 5.67 Hz, 1H) | 485 |
| 239 | 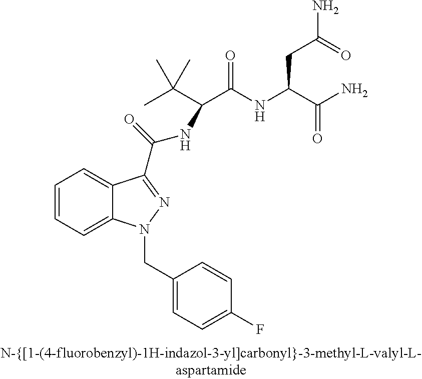 | ¹H NMR (400 MHz, DMSO-d₆) δ ppm 0.99 (s, 9H) 2.33-2.44 (m, 1H) 2.55 (s, 1H) 4.48-4.55 (m, 2H) 5.74-5.81 (m, 2H) 6.81 (br. s., 1H) 6.95 (br. s., 1H) 7.08-7.19 (m, 3H) 7.24-7.35 (m, 4H) 7.45 (t, J = 7.69 Hz, 1H) 7.63 (d, J = 9.52 Hz, 1H) 7.77 (d, J = 8.42 Hz, 1H) 8.17 (d, J = 8.42 Hz, 1H) 8.37 (d, J = 7.69 Hz, 1H) | 497 |
| 240 | 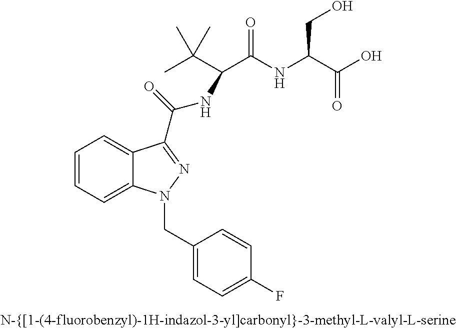 | ¹H NMR (400 MHz, DMSO-d6) δ ppm 1.00 (s, 9H) 2.53 (s, 1H) 3.60-3.73 (m, 2H) 4.23-4.34 (m, 1H) 4.65 (d, J = 10.25 Hz, 1H) 5.78 (s, 2H) 7.15 (t, J = 8.79 Hz, 2H) 7.24-7.34 (m, 3H) 7.45 (t, J = 7.69 Hz, 1H) 7.62 (d, J = 10.25 Hz, 1H) 7.79 (d, J = 8.79 Hz, 1H) 8.17 (d, J = 8.05 Hz, 1H) 8.52 (d, J = 7.32 Hz, 1H) | 471 |
| 241 | 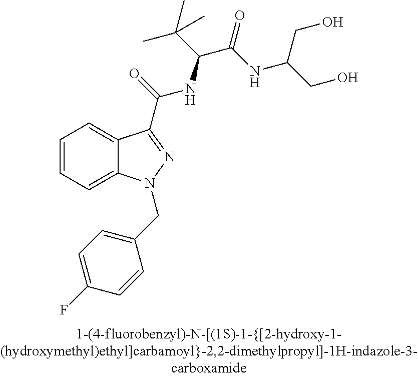 | ¹H NMR (400 MHz, DMSO-d6) δ ppm 0.97 (s, 9H) 3.43 (dq, J = 11.07, 5.58 Hz, 4H) 3.78 (d, J = 7.69 Hz, 1H) 4.52-4.60 (m, 3H) 5.77 (s, 2H) 7.15 (t, J = 8.97 Hz, 2H) 7.31 (dt, J = 8.24, 5.40 Hz, 3H) 7.45 (t, J = 7.69 Hz, 1H) 7.62 (d, J = 9.52 Hz, 1H) 7.77 (d, J = 8.79 Hz, 1H) 7.98 (d, J = 8.05 Hz, 1H) 8.18 (d, J = 8.05 Hz, 1H) | 457 |
| 242 | 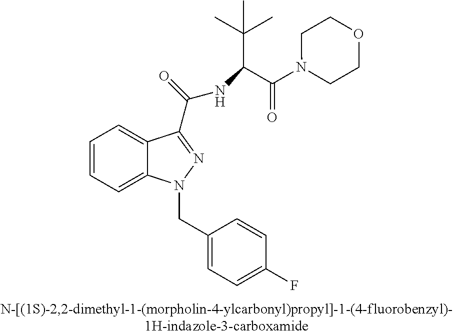 | ¹H NMR (400 MHz, DMSO-d₆) δ ppm 1.01 (s, 9H) 3.50-3.62 (m, 6H) 3.69 (dd, J = 9.34, 4.58 Hz, 2H) 5.04 (d, J = 9.52 Hz, 1H) 5.77 (s, 2H) 7.15 (t, J = 8.79 Hz, 2H) 7.31 (dt, J = 8.15, 5.26 Hz, 3H) 7.46 (t, J = 7.69 Hz, 1H) 7.62 (d, J = 9.52 Hz, 1H) 7.78 (d, J = 8.42 Hz, 1H) 8.16 (d, J = 8.05 Hz, 1H) | 453 |
| 243 | 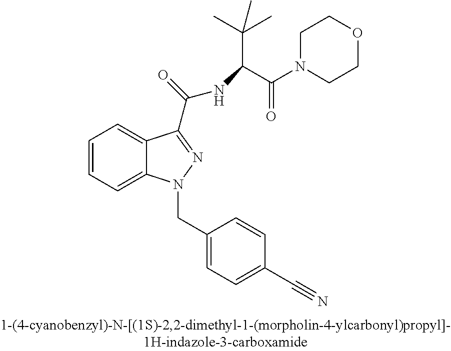 | ¹H NMR (400 MHz, DMSO-d₆) δ ppm 1.00 (s, 9H) 3.50-3.62 (m, 6H) 3.69 (dd, J = 10.07, 4.58 Hz, 2H) 5.04 (d, J = 9.52 Hz, 1H) 5.91 (d, J = 2.56 Hz, 2H) 7.31 (t, J = 7.51 Hz, 1H) 7.37 (d, J = 8.05 Hz, 2H) 7.47 (t, J = 7.51 Hz, 1H) 7.62 (d, J = 9.15 Hz, 1H) 7.76 (d, J = 8.42 Hz, 1H) 7.80 (d, J = 8.42 Hz, 2H) 8.18 (d, J = 8.05 Hz, 1H) | 460 |
| 244 | 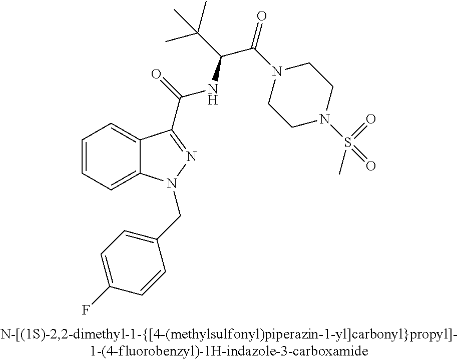 | ¹H NMR (400 MHz, DMSO-d6) δ ppm 1.01 (s, 9H) 2.87 (s, 3H) 3.02-3.24 (m, 4H) 3.50-3.60 (m, 1H) 3.69-3.80 (m, 3H) 3.82-3.91 (m, 1H) 5.08 (d, J = 9.52 Hz, 1H) 5.77 (d, J = 3.66 Hz, 2H) 7.15 (t, J = 9.15 Hz, 2H) 7.26-7.35 (m, 3H) 7.46 (t, J = 7.69 Hz, 1H) 7.64 (d, J = 9.52 Hz, 1H) 7.79 (d, J = 8.05 Hz, 1H) 8.17 (d, J = 8.05 Hz, 1H) | 530 |
| 245 | 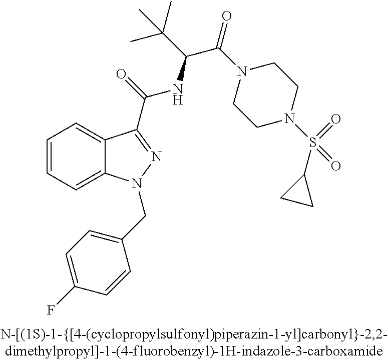 | ¹H NMR (400 MHz, DMSO-d6) δ ppm 0.85-0.98 (m, 4H) 1.01 (s, 9H) 2.55-2.64 (m, 1H) 3.09-3.26 (m, 4H) 3.48-3.59 (m, 1H) 3.69-3.80 (m, 2H) 3.87 (d, J = 13.91 Hz, 1H) 5.04-5.12 (m, 1H) 5.77 (d, J = 2.93 Hz, 2H) 7.15 (t, J = 8.79 Hz, 2H) 7.26-7.36 (m, 3H) 7.42-7.50 (m, 1H) 7.65 (d, J = 9.52 Hz, 1H) 7.79 (d, J = 8.05 Hz, 1H) 8.16 (d, J = 8.79 Hz, 1H) | 556 |
| 246 | 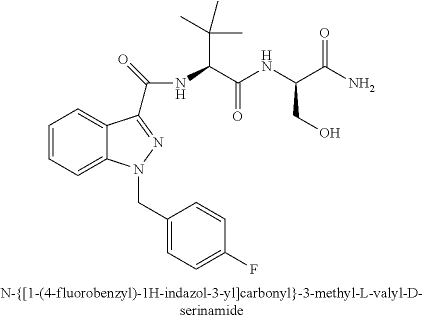 | ¹H NMR (400 MHz, DMSO-d₆) δ ppm 0.99 (s, 9H) 3.58 (t, J = 5.67 Hz, 2H) 4.23-4.35 (m, 1H) 4.57-4.64 (m, 1H) 4.82 (t, J = 5.31 Hz, 1H) 5.77 (s, 2H) 7.04 (br. s., 1H) 7.15 (t, J = 8.79 Hz, 2H) 7.26-7.35 (m, 4H) 7.45 (t, J = 7.69 Hz, 1H) 7.64 (d, J = 9.15 Hz, 1H) 7.74-7.84 (m, 1H) 8.17 (d, J = 8.05 Hz, 1H) 8.29 (d, J = 8.05 Hz, 1H) | 470 |
| 247 | 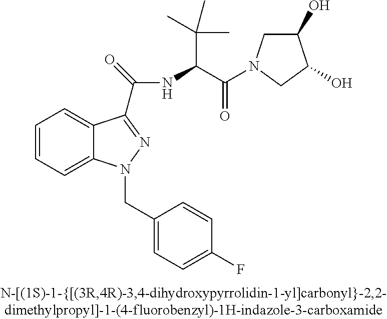 | ¹H NMR (400 MHz, DMSO-d₆) δ ppm 1.01 (s, 9H) 3.39 (d, J = 1.83 Hz, 2H) 3.56 (d, J = 10.62 Hz, 1H) 3.75 (dd, J = 10.80, 3.84 Hz, 1H) 3.91 (br. s., 1H) 4.00 (br. s., 1H) 4.77 (d, J = 9.88 Hz, 1H) 5.06 (d, J = 3.29 Hz, 1H) 5.18 (d, J = 3.29 Hz, 1H) 5.77 (s, 2H) 7.15 (t, J = 8.79 Hz, 2H) 7.26-7.34 (m, 3H) 7.45 (t, J = 7.69 Hz, 1H) 7.53 (d, J = 9.88 Hz, 1H) 7.77 (d, J = 8.79 Hz, 1H) 8.17 (d, J = 8.42 Hz, 1H) | 469 |
| 248 | 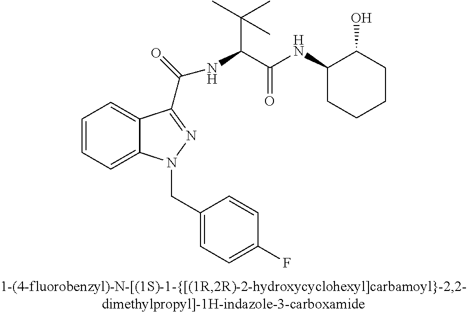 | ¹H NMR (400 MHz, DMSO-d₆) δ ppm 1.00 (s, 9H) 1.12-1.22 (m, 4H) 1.58 (d, J = 11.72 Hz, 2H) 1.75 (br. s., 1H) 1.82 (d, J = 10.25 Hz, 1H) 3.24 (d, J = 2.93 Hz, 1H) 3.47 (br. s., 1H) 4.39 (d, J = 5.86 Hz, 1H) 4.50 (d, J = 9.88 Hz, 1H) 5.77 (s, 2H) 7.15 (t, J = 8.79 Hz, 2H) 7.31 (dt, J = 8.05, 5.49 Hz, 3H) 7.45 (t, J = 7.69 Hz, 1H) 7.62 (d, J = 9.52 Hz, 1H) 7.77 (d, J = 8.42 Hz, 1H) 8.00 (d, J = 7.69 Hz, 1H) 8.18 (d, J = 8.05 Hz, 1H) | 481 |
| 249 | 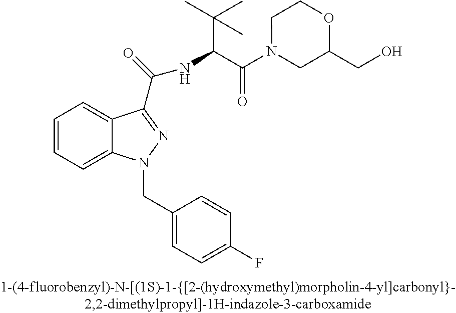 | ¹H NMR (400 MHz, DMSO-d₆) δ ppm 1.01 (d, J = 13.91 Hz, 9H) 2.77 (br. s., 1H) 2.94-3.05 (m, 1H) 3.15-3.24 (m, 1H) 3.34-3.46 (m, 3H) 3.81-3.93 (m, 1H) 4.06-4.26 (m, 1H) 4.37 (d, J = 14.28 Hz, 1H) 4.66-4.89 (m, 1H) 5.01-5.09 (m, 1H) 5.77 (s, 2H) 7.15 (t, J = 8.24 Hz, 2H) 7.27-7.33 (m, 3H) 7.46 (t, J = 7.51 Hz, 1H) 7.58-7.69 (m, 1H) 7.78 (dd, J = 8.24, 1.65 Hz, 1H) 8.16 (d, J = 8.42 Hz, 1H) | 483 |
| 250 | 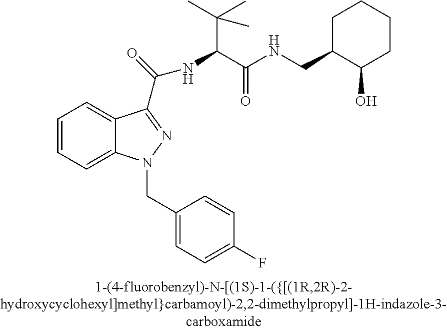 | ¹H NMR (400 MHz, DMSO-d₆) δ ppm 0.97 (s, 9H) 1.08-1.21 (m, 1H) 1.31 (br. s., 4H) 1.48 (d, J = 6.59 Hz, 1H) 1.54-1.62 (m, 2H) 2.86-2.95 (m, 1H) 3.03 (t, J = 6.22 Hz, 1H) 3.11-3.21 (m, 1H) 3.71 (br. s., 1H) 4.22 (d, J = 1.83 Hz, 1H) 4.49 (d, J = 9.88 Hz, 1H) 5.77 (s, 2H) 7.15 (t, J = 8.79 Hz, 2H) 7.26-7.35 (m, 3H) 7.45 (t, J = 7.69 Hz, 1H) 7.60 (d, J = 9.89 Hz, 1H) 7.78 (d, J = 8.42 Hz, 1H) 8.17 (d, J = 8.42 Hz, 2H) | 495 |
| 251 | 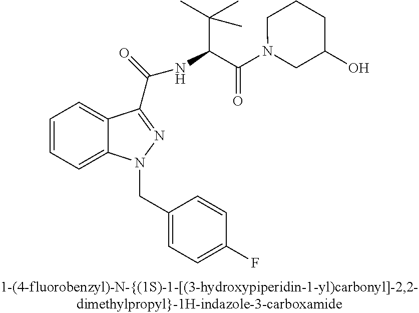 | ¹H NMR (400 MHz, DMSO-d₆) δ ppm 1.01 (d, J = 13.91 Hz, 9H) 2.77 (br. s., 1H) 2.94-3.05 (m, 1H) 3.15-3.24 (m, 1H) 3.34-3.46 (m, 2H) 3.81-3.93 (m, 1H) 4.05-4.25 (m, 1H) 4.37 (d, J = 14.28 Hz, 1H) 4.66-4.88 (m, 1H) 5.01-5.09 (m, 1H) 5.77 (s, 2H) 7.15 (t, J = 8.24 Hz, 2H) 7.27-7.33 (m, 3H) 7.46 (t, J = 7.51 Hz, 1H) 7.61 (dd, J = 9.34, 2.01 Hz, 1H) 7.66 (d, J = 9.52 Hz, 1H) 7.78 (dd, J = 8.24, 1.65 Hz, 1H) 8.16 (d, J = 8.42 Hz, 1H) | 467 |
| 252 | 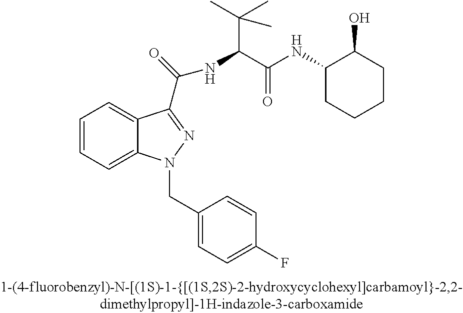 | ¹H NMR (400 MHz, DMSO-d₆) δ ppm 1.00 (s, 9H) 1.12-1.22 (m, 4H) 1.56 (br. s., 2H) 1.68-1.79 (m, 1H) 1.82 (d, J = 9.88 Hz, 1H) 3.24 (s, 1H) 3.47 (br. s., 1H) 4.39 (d, J = 5.49 Hz, 1H) 4.50 (d, J = 9.88 Hz, 1H) 5.77 (s, 2H) 7.15 (t, J = 8.79 Hz, 2H) 7.31 (dt, J = 8.05, 5.49 Hz, 3H) 7.45 (t, J = 7.69 Hz, 1H) 7.62 (d, J = 9.52 Hz, 1H) 7.77 (d, J = 8.42 Hz, 1H) 8.00 (d, J = 7.69 Hz, 1H) 8.18 (d, J = 8.05 Hz, 1H) | 481 |
| 253 | 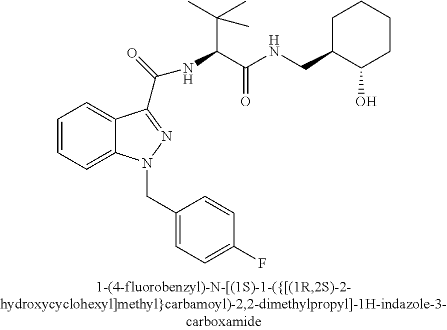 | ¹H NMR (400 MHz, DMSO-d₆) δ ppm 0.91 (d, J = 12.08 Hz, 1H) 0.98 (s, 9H) 1.06 (br. s., 1H) 1.12 (d, J = 10.62 Hz, 2H) 1.25 (s, 1H) 1.54 (d, J = 12.08 Hz, 1H) 1.61 (br. s., 1H) 1.69-1.81 (m, 2H) 2.94 (d, J = 7.32 Hz, 1H) 3.07 (dd, J = 8.42, 5.13 Hz, 1H) 3.47 (d, J = 12.08 Hz, 1H) 4.48-4.55 (m, 2H) 5.77 (s, 2H) 7.15 (t, J = 8.79 Hz, 2H) 7.26-7.36 (m, 3H) 7.45 (t, J = 7.69 Hz, 1H) 7.60 (d, J = 9.52 Hz, 1H) 7.78 (d, J = 8.42 Hz, 1H) 8.11 (t, J = 5.49 Hz, 1H) 8.17 (d, J = 8.42 Hz, 1H) | 495 |
| 254 | 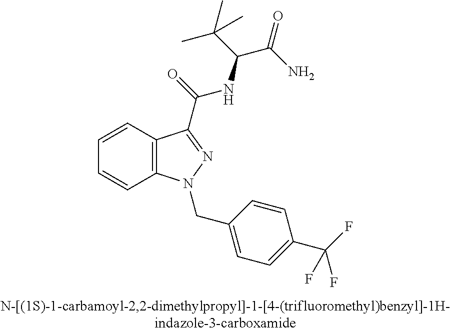 | ¹H NMR (400 MHz, DMSO-d6) δ ppm 0.99 (s, 9H) 4.46 (d, J = 9.52 Hz, 1H) 5.92 (s, 2H) 7.21 (br. s., 1H) 7.30 (t, J = 7.51 Hz, 1H) 7.39-7.49 (m, 3H) 7.60 (d, J = 9.89 Hz, 1H) 7.66-7.72 (m, 3H) 7.76 (d, J = 8.42 Hz, 1H) 8.20 (d, J = 8.42 Hz, 1H) | 433 |
| 255 | 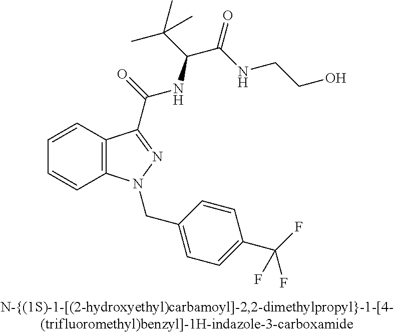 | ¹H NMR (400 MHz, DMSO-d6) δ ppm 0.97 (s, 9H) 3.13 (d, J = 5.86 Hz, 1H) 3.17-3.24 (m, 1H) 3.42 (q, J = 5.86 Hz, 2H) 4.50 (d, J = 9.88 Hz, 1H) 4.60 (t, J = 5.31 Hz, 1H) 5.92 (s, 2H) 7.30 (t, J = 7.51 Hz, 1H) 7.39-7.49 (m, 3H) 7.61 (d, J = 9.52 Hz, 1H) 7.70 (d, J = 8.05 Hz, 2H) 7.76 (d, J = 8.42 Hz, 1H) 8.19 (d, J = 8.42 Hz, 1H) 8.25 (t, J = 5.31 Hz, 1H) | 477 |
| 256 | 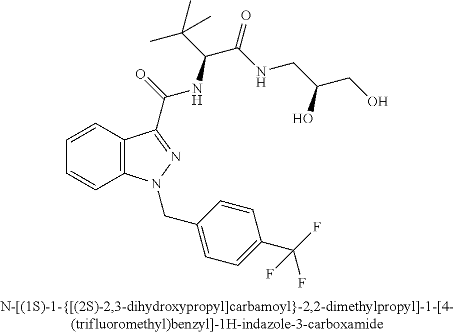 | ¹H NMR (400 MHz, DMSO-d6) δ ppm 0.98 (s, 10H) 2.98 (d, J = 5.86 Hz, 1H) 3.30 (d, J = 3.66 Hz, 1H) 3.52 (d, J = 6.59 Hz, 1H) 4.47 (t, J = 5.67 Hz, 1H) 4.55 (d, J = 9.52 Hz, 1H) 4.64 (d, J = 4.76 Hz, 1H) 5.92 (s, 2H) 7.30 (t, J = 7.51 Hz, 1H) 7.39-7.49 (m, 3H) 7.62 (d, J = 9.88 Hz, 1H) 7.70 (d, J = 8.42 Hz, 2H) 7.76 (d, J = 8.42 Hz, 1H) 8.17-8.26 (m, 2H) | 507 |
| 257 | 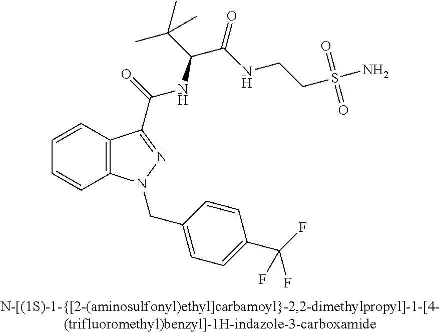 | ¹H NMR (400 MHz, DMSO-d6) δ ppm 0.98 (s, 9H) 3.08-3.19 (m, 2H) 3.42-3.54 (m, 2H) 4.45 (d, J = 9.52 Hz, 1H) 5.92 (s, 2H) 6.86 (s, 2H) 7.31 (t, J = 7.51 Hz, 1H) 7.40-7.49 (m, 3H) 7.60 (d, J = 9.52 Hz, 1H) 7.70 (d, J = 8.05 Hz, 2H) 7.77 (d, J = 8.79 Hz, 1H) 8.19 (d, J = 8.05 Hz, 1H) 8.46 (t, J = 5.49 Hz, 1H) | 540 |
| 258 | 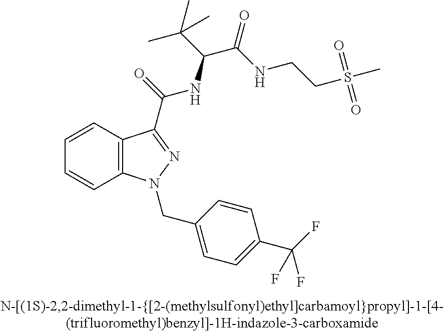 | ¹H NMR (400 MHz, DMSO-d6) δ ppm 0.98 (s, 10H) 2.99 (s, 3H) 3.44-3.55 (m, 2H) 4.47 (d, J = 9.52 Hz, 1H) 5.92 (s, 2H) 7.31 (t, J = 7.51 Hz, 1H) 7.42 (m, J = 8.05 Hz, 2H) 7.47 (t, J = 7.51 Hz, 1H) 7.60 (d, J = 9.52 Hz, 1H) 7.70 (m, J = 8.42 Hz, 2H) 7.77 (d, J = 8.42 Hz, 1H) 8.18 (d, J = 8.42 Hz, 1H) 8.54 (t, J = 5.31 Hz, 1H) | 539 |
| 259 | 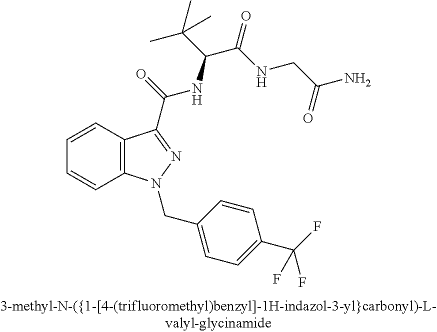 | ¹H NMR (400 MHz, DMSO-d6) δ ppm 0.84 (s, 1H) 0.95-1.02 (m, 9H) 3.69 (d, J = 5.86 Hz, 2H) 4.54 (d, J = 9.15 Hz, 1H) 5.92 (s, 2H) 6.94 (br. s., 1H) 7.23-7.34 (m, 2H) 7.39-7.49 (m, 3H) 7.63 (d, J = 9.52 Hz, 1H) 7.70 (d, J = 8.05 Hz, 2H) 7.76 (d, J = 8.79 Hz, 1H) 8.19 (d, J = 8.42 Hz, 1H) 8.43 (t, J = 5.67 Hz, 1H) | 490 |
| 260 | 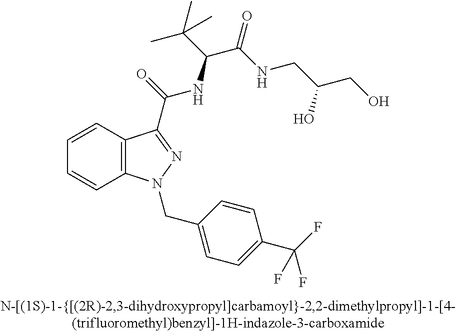 | ¹H NMR (400 MHz, DMSO-d6) δ ppm 0.98 (s, 9H) 3.04-3.12 (m, 1H) 3.15-3.24 (m, 1H) 3.29-3.33 (m, 1H) 3.50 (d, J = 5.86 Hz, 1H) 4.46 (t, J = 5.67 Hz, 1H) 4.54 (d, J = 9.88 Hz, 1H) 4.66 (d, J = 5.13 Hz, 1H) 5.92 (s, 2H) 7.30 (t, J = 7.51 Hz, 1H) 7.39-7.49 (m, 3H) 7.61 (d, J = 9.88 Hz, 1H) 7.70 (d, J = 8.05 Hz, 2H) 7.76 (d, J = 8.42 Hz, 1H) 8.17-8.25 (m, 2H) | 507 |
| 261 | 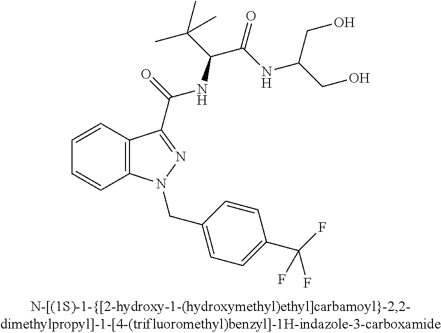 | ¹H NMR (400 MHz, DMSO-d6) δ ppm 0.97 (s, 9H) 3.38-3.48 (m, 4H) 3.77 (d, J = 7.69 Hz, 1H) 4.53-4.59 (m, 3H) 5.92 (s, 2H) 7.30 (t, J = 7.51 Hz, 1H) 7.41 (m, J = 8.05 Hz, 2H) 7.46 (t, J = 7.69 Hz, 1H) 7.62 (d, J = 9.88 Hz, 1H) 7.70 (m, J = 8.05 Hz, 2H) 7.76 (d, J = 8.79 Hz, 1H) 7.98 (d, J = 8.05 Hz, 1H) 8.20 (d, J = 8.05 Hz, 1H) | 507 |
| 262 | 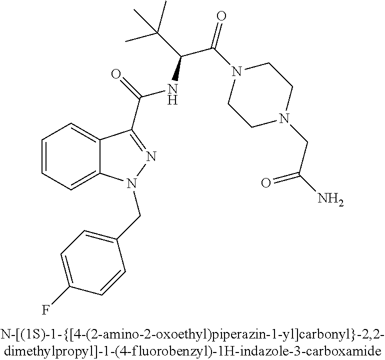 | ¹H NMR (400 MHz, DMSO-d6) δ ppm 1.00 (s, 9H) 2.42 (br. s., 2H) 2.89 (br. s., 2H) 3.24 (none, 1H) 3.56 (d, J = 1.46 Hz, 2H) 3.72 (br. s., 2H) 5.07 (d, J = 9.52 Hz, 1H) 5.77 (s, 2H) 7.08 (br. s., 1H) 7.15 (t, J = 8.79 Hz, 2H) 7.23 (br. s., 1H) 7.26-7.37 (m, 3H) 7.46 (t, J = 7.69 Hz, 1H) 7.62 (d, J = 9.52 Hz, 1H) 7.78 (d, J = 8.05 Hz, 1H) 8.17 (d, J = 8.05 Hz, 1H) | 509 |
| 263 | 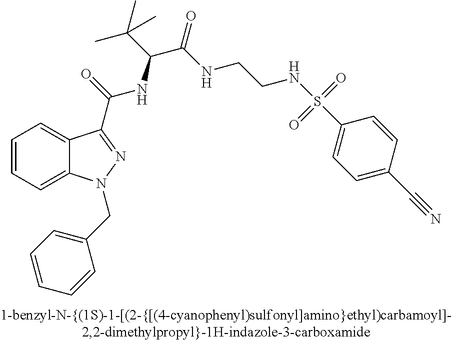 | ¹H NMR (400 MHz, DMSO-d6) δ ppm 0.95 (s, 9H) 2.84 (q, J = 6.59 Hz, 2H) 3.15 (td, J = 12.63, 6.59 Hz, 2H) 4.42 (d, J = 9.52 Hz, 1H) 5.78 (s, 2H) 7.23-7.34 (m, 6H) 7.45 (t, J = 7.69 Hz, 1H) 7.57 (d, J = 9.52 Hz, 1H) 7.76 (d, J = 8.42 Hz, 1H) 7.91-7.99 (m, 3H) 8.05 (d, J = 8.42 Hz, 2H) 8.17 (d, J = 8.05 Hz, 1H) 8.32 (t, J = 5.49 Hz, 1H) | 573 |
| 264 | 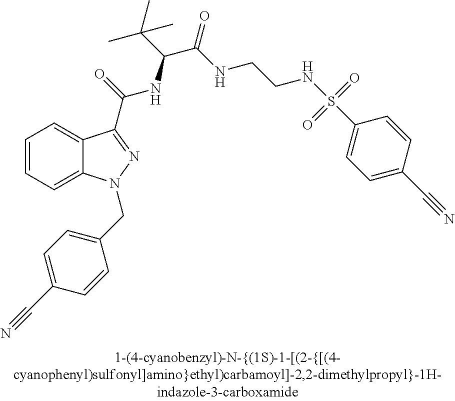 | ¹H NMR (400 MHz, DMSO-d6) δ ppm 0.95 (s, 9H) 2.84 (br. s., 2H) 3.09-3.21 (m, 2H) 4.43 (d, J = 9.52 Hz, 1H) 5.91 (s, 2H) 7.28-7.39 (m, 3H) 7.47 (t, J = 7.69 Hz, 1H) 7.57 (d, J = 9.52 Hz, 1H) 7.75 (d, J = 8.42 Hz, 1H) 7.79 (d, J = 8.05 Hz, 2H) 7.91-7.99 (m, 3H) 8.05 (d, J = 8.42 Hz, 2H) 8.18 (d, J = 8.05 Hz, 1H) 8.32 (t, J = 5.49 Hz, 1H) | 598 |
| 265 | 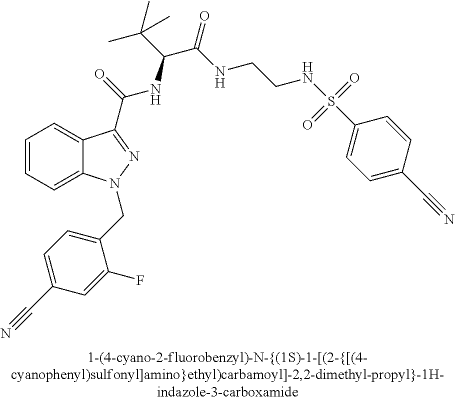 | ¹H NMR (400 MHz, DMSO-d6) δ ppm 0.93 (s, 9H) 2.84 (d, J = 5.49 Hz, 2H) 3.08-3.20 (m, 2H) 4.41 (d, J = 9.52 Hz, 1H) 5.93 (s, 2H) 7.19 (t, J = 7.69 Hz, 1H) 7.31 (t, J = 7.51 Hz, 1H) 7.48-7.56 (m, 2H) 7.63 (d, J = 7.69 Hz, 1H) 7.77 (d, J = 8.42 Hz, 1H) 7.93 (q, J = 7.69 Hz, 4H) 8.05 (d, J = 8.42 Hz, 2H) 8.18 (d, J = 8.05 Hz, 1H) 8.32 (t, J = 5.31 Hz, 1H) | 616 |
| 266 | 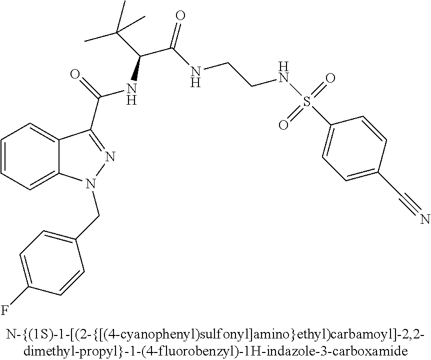 | ¹H NMR (400 MHz, DMSO-d6) δ ppm 0.95 (s, 9H) 2.85 (t, J = 6.41 Hz, 2H) 3.15 (td, J = 13.27, 6.77 Hz, 2H) 4.42 (d, J = 9.88 Hz, 1H) 5.77 (s, 2H) 7.15 (t, J = 8.79 Hz, 2H) 7.27-7.36 (m, 3H) 7.45 (t, J = 7.69 Hz, 1H) 7.57 (d, J = 9.52 Hz, 1H) 7.77 (d, J = 8.79 Hz, 1H) 7.91-7.99 (m, 3H) 8.05 (d, J = 8.42 Hz, 2H) 8.16 (d, J = 8.05 Hz, 1H) 8.32 (t, J = 5.49 Hz, 1H) | 591 |
| 267 | 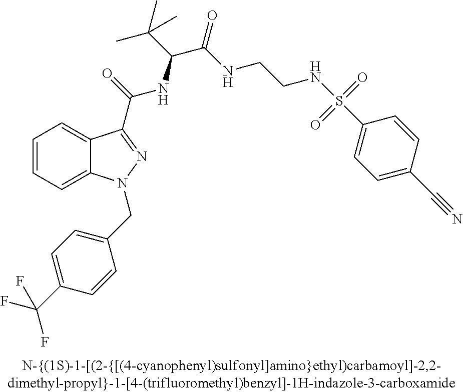 | ¹H NMR (400 MHz, DMSO-d6) δ ppm 0.95 (s, 9H) 2.85 (t, J = 6.59 Hz, 2H) 3.15 (td, J = 13.55, 6.59 Hz, 2H) 4.43 (d, J = 9.52 Hz, 1H) 5.91 (s, 2H) 7.30 (t, J = 7.51 Hz, 1H) 7.39-7.49 (m, 3H) 7.57 (d, J = 9.52 Hz, 1H) 7.69 (d, J = 8.05 Hz, 2H) 7.76 (d, J = 8.79 Hz, 1H) 7.94 (t, J = 8.05 Hz, 3H) 8.05 (d, J = 8.42 Hz, 2H) 8.18 (d, J = 8.42 Hz, 1H) 8.32 (t, J = 5.31 Hz, 1H) | 641 |
| 268 | 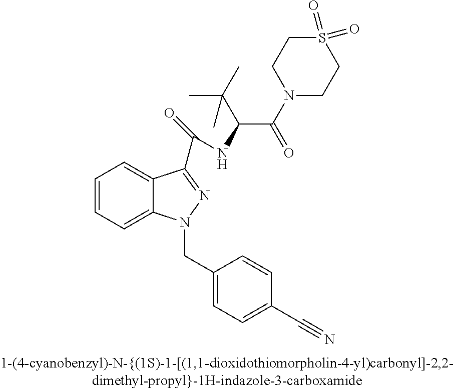 | ¹H NMR (400 MHz, DMSO-d₆) δ ppm 1.02 (s, 9H) 3.08-3.20 (m, 3H) 3.33 (br. s., 1H) 3.52 (br. s., 1H) 3.80-3.88 (m, 1H) 4.37 (t, J = 17.39 Hz, 2H) 5.06 (d, J = 8.79 Hz, 1H) 5.91 (s, 2H) 7.31 (t, J = 7.51 Hz, 1H) 7.39 (d, J = 8.42 Hz, 2H) 7.47 (t, J = 7.69 Hz, 1H) 7.75 (dd, J = 13.55, 8.79 Hz, 2H) 7.80 (d, J = 8.05 Hz, 2H) 8.18 (d, J = 8.05 Hz, 1H) | 508 |
| 269 | 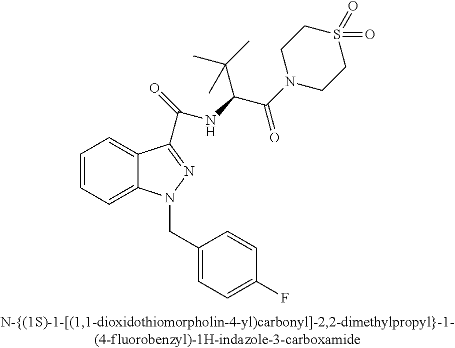 | ¹H NMR (400 MHz, DMSO-d₆) δ ppm 1.02 (s, 9H) 3.14 (br. s., 3H) 3.33 (br. s., 1H) 3.52 (br. s., 1H) 3.84 (br. s., 1H) 4.37 (t, J = 16.84 Hz, 2H) 5.05 (d, J = 9.15 Hz, 1H) 5.77 (s, 2H) 7.15 (t, J = 8.79 Hz, 2H) 7.27-7.36 (m, 3H) 7.46 (t, J = 7.69 Hz, 1H) 7.73 (d, J = 8.79 Hz, 1H) 7.79 (d, J = 8.79 Hz, 1H) 8.16 (d, J = 8.05 Hz, 1H) | 501 |
| 270 | 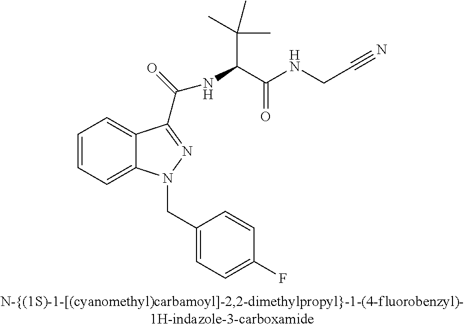 | ¹H NMR (400 MHz, DMSO-d₆) δ ppm 0.99 (s, 9H) 4.19 (dd, J = 11.72, 5.49 Hz, 2H) 4.53 (d, J = 9.52 Hz, 1H) 5.78 (s, 2H) 7.15 (t, J = 8.79 Hz, 2H) 7.27-7.37 (m, 3H) 7.46 (t, J = 7.69 Hz, 1H) 7.61 (d, J = 9.88 Hz, 1H) 7.78 (d, J = 8.42 Hz, 1H) 8.16 (d, J = 8.42 Hz, 1H) 9.03 (t, J = 5.31 Hz, 1H) | 422 |
| 271 | 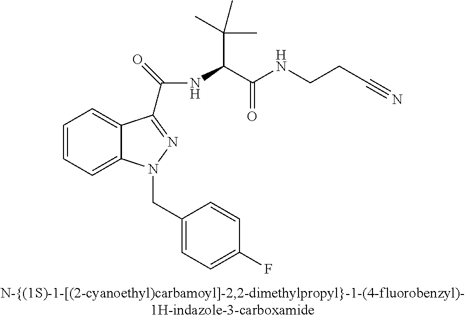 | ¹H NMR (400 MHz, DMSO-d₆) δ ppm 0.99 (s, 9H) 2.62-2.72 (m, 2H) 3.21-3.27 (m, 1H) 3.37-3.46 (m, 1H) 4.49 (d, J = 9.52 Hz, 1H) 5.77 (s, 2H) 7.15 (t, J = 8.79 Hz, 2H) 7.26-7.36 (m, 3H) 7.45 (t, J = 7.69 Hz, 1H) 7.60 (d, J = 9.88 Hz, 1H) 7.77 (d, J = 8.42 Hz, 1H) 8.17 (d, J = 8.05 Hz, 1H) 8.64 (t, J = 5.31 Hz, 1H) | 436 |
| 272 | 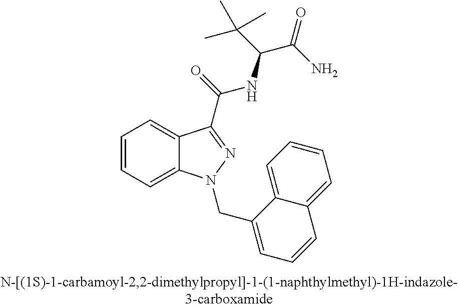 | ¹H NMR (400 MHz, DMSO-d₆) δ ppm 0.98 (s, 9H) 4.45 (d, J = 9.88 Hz, 1H) 6.29 (d, J = 5.49 Hz, 2H) 7.13 (d, J = 6.96 Hz, 1H) 7.20 (br. s., 1H) 7.28 (t, J = 7.51 Hz, 1H) 7.42 (t, J = 7.69 Hz, 2H) 7.54-7.64 (m, 3H) 7.67 (br. s., 1H) 7.76 (d, J = 8.42 Hz, 1H) 7.88 (d, J = 8.05 Hz, 1H) 7.95 (d, J = 7.32 Hz, 1H) 8.18 (d, J = 8.05 Hz, 1H) 8.41 (d, J = 7.69 Hz, 1H) | 415 |
| 273 | 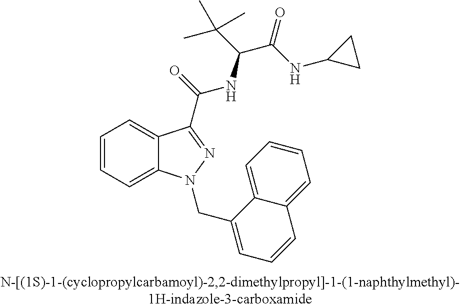 | ¹H NMR (400 MHz, DMSO-d₆) δ ppm 0.35-0.46 (m, 2H) 0.62 (dd, J = 4.58, 2.38 Hz, 2H) 0.95 (s, 9H) 2.66 (dd, J = 7.14, 3.48 Hz, 1H) 4.38 (d, J = 9.88 Hz, 1H) 6.29 (d, J = 6.96 Hz, 2H) 7.14 (d, J = 6.96 Hz, 1H) 7.28 (t, J = 7.51 Hz, 1H) 7.42 (t, J = 7.51 Hz, 2H) 7.53-7.63 (m, 3H) 7.77 (d, J = 8.79 Hz, 1H) 7.88 (d, J = 8.42 Hz, 1H) 7.96 (d, J = 7.32 Hz, 1H) 8.17 (d, J = 8.05 Hz, 1H) 8.29 (d, J = 4.03 Hz, 1H) 8.41 (d, J = 7.69 Hz, 1H) | 455 |
| 274 | 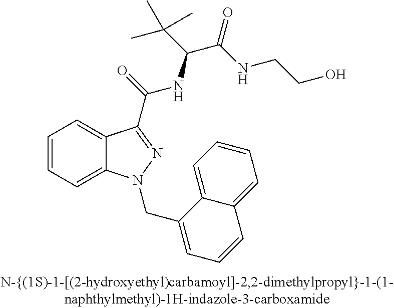 | ¹H NMR (400 MHz, DMSO-d₆) δ ppm 0.97 (s, 9H) 3.14 (d, J = 5.49 Hz, 1H) 3.17-3.24 (m, 1H) 3.42 (q, J = 5.86 Hz, 2H) 4.48 (d, J = 9.88 Hz, 1H) 4.61 (t, J = 5.31 Hz, 1H) 6.29 (d, J = 6.96 Hz, 2H) 7.14 (d, J = 6.96 Hz, 1H) 7.28 (t, J = 7.51 Hz, 1H) 7.42 (t, J = 7.69 Hz, 2H) 7.53-7.65 (m, 3H) 7.76 (d, J = 8.79 Hz, 1H) 7.88 (d, J = 8.42 Hz, 1H) 7.96 (d, J = 7.32 Hz, 1H) 8.17 (d, J = 8.42 Hz, 1H) 8.24 (t, J = 5.31 Hz, 1H) 8.41 (d, J = 8.42 Hz, 1H) | 459 |
| 275 | 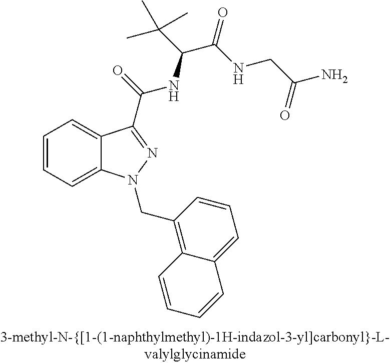 | ¹H NMR (400 MHz, DMSO-d₆) δ ppm 0.99 (s, 9H) 3.69 (d, J = 5.49 Hz, 2H) 4.51 (d, J = 9.52 Hz, 1H) 6.29 (d, J = 5.49 Hz, 2H) 6.95 (br. s., 1H) 7.14 (d, J = 6.96 Hz, 1H) 7.21-7.33 (m, 2H) 7.42 (t, J = 7.69 Hz, 2H) 7.53-7.61 (m, 2H) 7.65 (d, J = 9.15 Hz, 1H) 7.76 (d, J = 8.42 Hz, 1H) 7.88 (d, J = 8.05 Hz, 1H) 7.96 (d, J = 7.32 Hz, 1H) 8.17 (d, J = 8.05 Hz, 1H) 8.38-8.47 (m, 2H) | 472 |
| 276 | 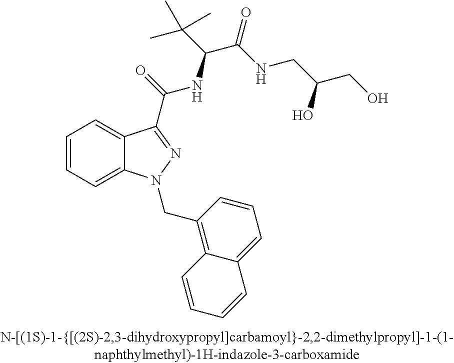 | ¹H NMR (400 MHz, DMSO-d₆) δ ppm 0.97 (s, 9H) 2.93-3.02 (m, 1H) 3.29-3.35 (m, 3H) 3.52 (d, J = 5.86 Hz, 1H) 4.47 (t, J = 5.67 Hz, 1H) 4.53 (d, J = 9.52 Hz, 1H) 4.64 (d, J = 4.76 Hz, 1H) 6.29 (d, J = 6.22 Hz, 2H) 7.14 (d, J = 6.96 Hz, 1H) 7.28 (t, J = 7.51 Hz, 1H) 7.42 (t, J = 7.51 Hz, 2H) 7.57 (t, J = 6.59 Hz, 2H) 7.65 (d, J = 9.52 Hz, 1H) 7.76 (d, J = 8.42 Hz, 1H) 7.88 (d, J = 8.42 Hz, 1H) 7.95 (d, J = 6.96 Hz, 1H) 8.15-8.24 (m, 2H) 8.41 (d, J = 8.05 Hz, 1H) | 489 |
| 277 | 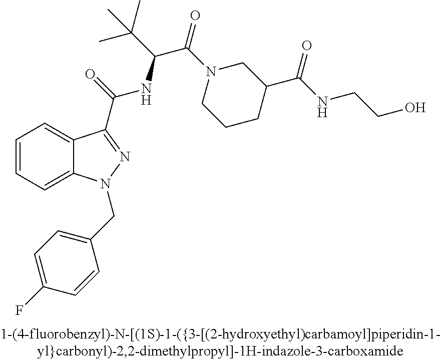 | ¹H NMR (400 MHz, DMSO-d6) δ ppm 1.00 (d, J = 13.91 Hz, 9H) 1.42 (d, J = 24.90 Hz, 1H) 1.49-1.95 (m, 4H) 2.55-2.83 (m, 2H) 2.96-3.19 (m, 3H) 3.31-3.45 (m, 2H) 4.14 (br. s., 1H) 4.31-4.49 (m, 1H) 4.51-4.67 (m, 1H) 5.09 (dd, J = 6.59, 2.93 Hz, 1H) 5.77 (br. s., 2H) 7.10-7.21 (m, 2H) 7.24-7.38 (m, 3H) 7.46 (dd, J = 12.08, 7.69 Hz, 1H) 7.55-7.64 (m, 1H) 7.74-7.83 (m, 1H) 8.12-8.22 (m, 1H) | 538 |
| 278 | 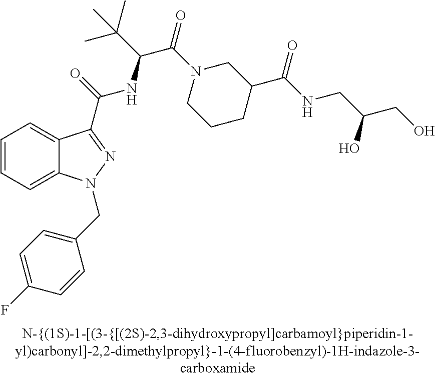 | ¹H NMR (400 MHz, DMSO-d6) δ ppm 1.00 (d, J = 14.65 Hz, 9H) 1.59 (dd, J = 12.08, 3.30 Hz, 2H) 1.77 (br. s., 1H) 2.18-2.35 (m, 1H) 2.55-2.65 (m, 1H) 2.70-2.81 (m, 1H) 2.88-3.03 (m, 2H) 3.03-3.17 (m, 2H) 3.38-3.58 (m, 2H) 4.16 (d, J = 2.93 Hz, 1H) 4.39-4.50 (m, 2H) 4.59-4.72 (m, 1H) 5.02-5.12 (m, 1H) 5.77 (br. s., 2H) 7.11-7.20 (m, 2H) 7.25-7.37 (m, 3H) 7.46 (dd, J = 12.08, 7.69 Hz, 1H) 7.55-7.64 (m, 1H) 7.73-7.83 (m, 2H) 8.17 (dd, J = 7.69, 4.03 Hz, 1H) | 568 |
| 279 | 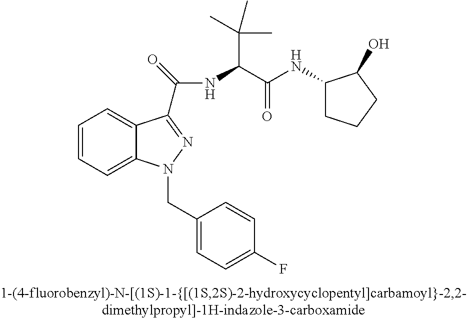 | ¹H NMR (400 MHz, DMSO-d₆) δ ppm 0.98 (s, 9H) 1.32-1.42 (m, 1H) 1.45 (br. s., 1H) 1.63 (t, J = 7.32 Hz, 2H) 1.76 (d, J = 8.42 Hz, 1H) 1.87-1.96 (m, 1H) 3.83 (d, J = 4.76 Hz, 2H) 4.50 (d, J = 9.52 Hz, 1H) 4.65 (d, J = 4.03 Hz, 1H) 5.77 (s, 2H) 7.15 (t, J = 8.79 Hz, 2H) 7.26-7.36 (m, 3H) 7.45 (t, J = 7.51 Hz, 1H) 7.58 (d, J = 9.52 Hz, 1H) 7.77 (d, J = 8.79 Hz, 1H) 8.10 (d, J = 6.96 Hz, 1H) 8.17 (d, J = 8.42 Hz, 1H) | 467 |
| 280 | 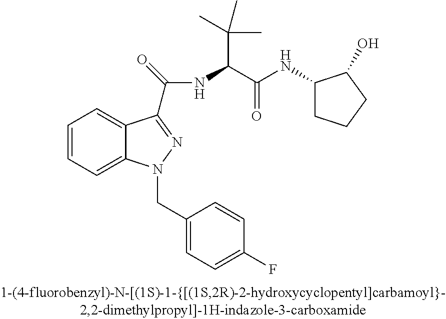 | ¹H NMR (400 MHz, DMSO-d₆) δ ppm 0.99 (s, 9H) 1.45 (d, J = 1.46 Hz, 1H) 1.52-1.61 (m, 2H) 1.66-1.78 (m, 3H) 3.85-3.97 (m, 2H) 4.50 (d, J = 4.03 Hz, 1H) 4.59 (d, J = 9.52 Hz, 1H) 5.77 (s, 2H) 7.15 (t, J = 8.97 Hz, 2H) 7.26-7.36 (m, 3H) 7.45 (t, J = 7.69 Hz, 1H) 7.65 (d, J = 9.52 Hz, 1H) 7.77 (d, J = 8.42 Hz, 1H) 7.84 (d, J = 7.32 Hz, 1H) 8.18 (d, J = 8.42 Hz, 1H) | 467 |
| 281 | 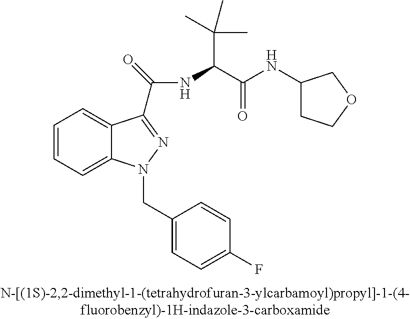 | ¹H NMR (400 MHz, DMSO-d₆) δ ppm 0.97 (d, J = 2.93 Hz, 9H) 1.69-1.79 (m, 1H) 2.08 (td, J = 11.99, 7.51 Hz, 1H) 3.47 (dd, J = 8.79, 3.66 Hz, 1H) 3.65-3.69 (m, 1H) 3.71-3.80 (m, 2H) 4.22-4.31 (m, 1H) 4.51 (d, J = 9.88 Hz, 1H) 5.77 (s, 2H) 7.15 (t, J = 8.97 Hz, 2H) 7.28-7.36 (m, 3H) 7.45 (t, J = 7.51 Hz, 1H) 7.59 (dd, J = 9.70, 2.38 Hz, 1H) 7.78 (d, J = 8.42 Hz, 1H) 8.17 (d, J = 8.42 Hz, 1H) 8.47 (t, J = 6.77 Hz, 1H) | 453 |
| 282 | 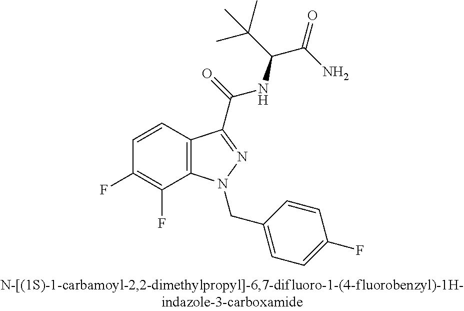 | ¹H NMR (400 MHz, DMSO-d6) δ ppm 7.94-8.02 (m, 1H), 7.69 (br. s., 1H), 7.58-7.66 (m, 1H), 7.31-7.41 (m, 1H), 7.21-7.31 (m, 3H), 7.13-7.21 (m, 2H), 5.78 (s, 2H), 4.45 (d, J = 9.52 Hz, 1H), 0.99 (s, 9H) | 419 |
| 283 | 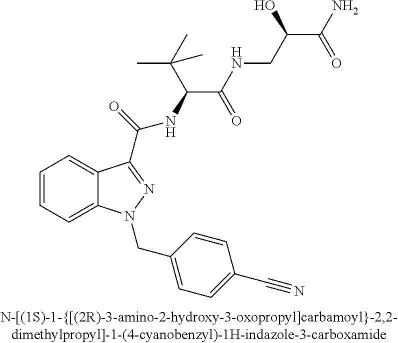 | ¹H NMR (400 MHz, DMSO-d₆) δ ppm 0.98 (s, 9H) 3.11 (s, 1H) 3.46-3.53 (m, 1H) 3.88-3.98 (m, 1H) 4.56 (d, J = 9.88 Hz, 1H) 5.54 (d, J = 5.49 Hz, 1H) 5.91 (s, 2H) 7.17 (d, J = 8.42 Hz, 2H) 7.30 (t, J = 7.69 Hz, 1H) 7.37 (m, 2H) 7.46 (t, J = 7.51 Hz, 1H) 7.62 (d, J = 9.52 Hz, 1H) 7.75 (d, J = 8.42 Hz, 1H) 7.79 (m, J = 8.42 Hz, 2H) 8.19 (d, J = 8.05 Hz, 1H) 8.25 (t, J = 5.49 Hz, 1H) | 477 |
| 284 | 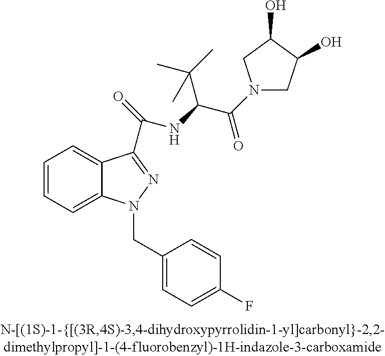 | ¹H NMR (400 MHz, DMSO-d₆) δ ppm 1.00 (d, J = 3.66 Hz, 9H) 3.16-3.28 (m, 1H) 3.38 (d, J = 5.49 Hz, 1H) 3.41-3.49 (m, 1H) 3.77 (td, J = 10.34, 6.04 Hz, 1H) 3.98-4.10 (m, 2H) 4.75 (dd, J = 13.00, 9.70 Hz, 1H) 4.82-4.92 (m, 1H) 4.99 (dd, J = 11.17, 5.31 Hz, 1H) 5.72-5.81 (m, 2H) 7.15 (t, J = 8.79 Hz, 2H) 7.26-7.36 (m, 3H) 7.45 (t, J = 7.69 Hz, 1H) 7.53 (d, J = 9.15 Hz, 1H) 7.77 (dd, J = 8.42, 5.49 Hz, 1H) 8.17 (dd, J = 8.05, 4.03 Hz, 1H) | 469 |
| 285 | 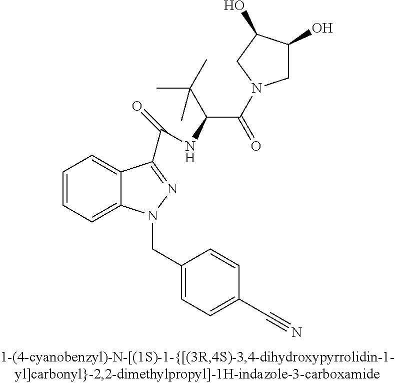 | ¹H NMR (400 MHz, DMSO-d₆) δ ppm 0.99 (d, J = 3.66 Hz, 8H) 3.06 (s, 1H) 3.14-3.27 (m, 1H) 3.42-3.50 (m, 1H) 3.74-3.82 (m, 1H) 3.97-4.05 (m, 1H) 4.07 (br. s., 1H) 4.75 (dd, J = 13.36, 9.70 Hz, 1H) 4.89 (br. s., 1H) 4.94-5.06 (m, 1H) 5.84 (d, J = 7.32 Hz, 1H) 5.88-5.96 (m, 2H) 7.29-7.40 (m, 3H) 7.42-7.49 (m, 1H) 7.53 (d, J = 9.52 Hz, 1H) 7.73-7.82 (m, 3H) 8.19 (dd, J = 8.05, 4.03 Hz, 1H) | 476 |
| 286 | 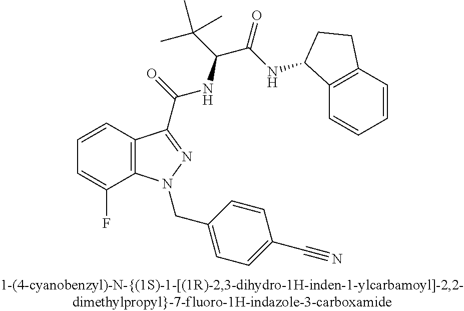 | ¹H NMR (400 MHz, DMSO-d6) δ ppm 8.63 (d, J = 8.05 Hz, 1H) 8.03 (d, J = 7.32 Hz, 1H) 7.81 (d, J = 8.42 Hz, 2H) 7.71 (d, J = 9.88 Hz, 1H) 7.10-7.39 (m, 8H) 5.95 (s, 2H) 5.31 (q, J = 7.57 Hz, 1H) 4.56 (d, J = 9.52 Hz, 1H) 2.89-3.00 (m, 1H) 2.75-2.86 (m, 1H) 2.35-2.45 (m, J = 12.17, 8.01, 8.01, 4.03 Hz, 1H) 1.74-1.86 (m, J = 12.81, 8.05, 7.87, 7.87 Hz, 1H) 1.01 (s, 9H). | 524 |
| 287 | 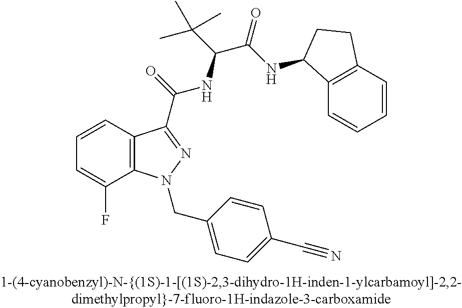 | ¹H NMR (400 MHz, DMSO-d6) δ ppm 8.61 (d, J = 8.05 Hz, 1H) 8.00-8.07 (m, 1H) 7.81 (d, J = 8.42 Hz, 2H) 7.71 (d, J = 9.88 Hz, 1H) 7.15-7.38 (m, 8H) 5.94 (s, 2H) 5.33 (q, J = 7.69 Hz, 1H) 4.55 (d, J = 9.88 Hz, 1H) 2.90-3.01 (m, 1H) 2.79 (ddd, J = 16.02, 8.42, 8.15 Hz, 1H) 2.32-2.43 (m, J = 12.40, 8.26, 8.26, 3.66 Hz, 1H) 1.77-1.90 (m, J = 12.49, 8.42, 8.21, 8.21 Hz, 1H) 1.01 (s, 9H). | 524 |
| 288 | 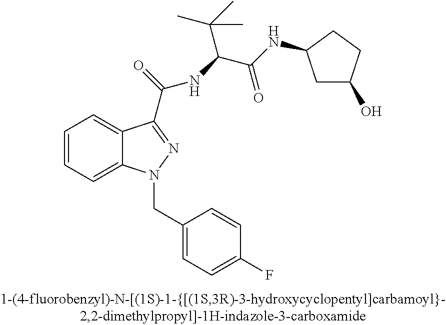 | ¹H NMR (400 MHz, DMSO-d₆) δ ppm 0.97 (s, 9H) 1.26-1.39 (m, 1H) 1.49-1.68 (m, 3H) 1.78 (br. s., 1H) 2.13 (d, J = 5.86 Hz, 1H) 3.98 (d, J = 7.32 Hz, 1H) 4.04 (d, J = 4.76 Hz, 1H) 4.48 (d, J = 9.52 Hz, 1H) 4.57 (t, J = 3.66 Hz, 1H) 5.77 (s, 2H) 7.15 (t, J = 8.79 Hz, 2H) 7.31 (dt, J = 8.15, 5.26 Hz, 3H) 7.45 (t, J = 7.69 Hz, 1H) 7.59 (d, J = 9.52 Hz, 1H) 7.77 (d, J = 8.79 Hz, 1H) 8.16-8.26 (m, 2H) | 467 |
| 289 | 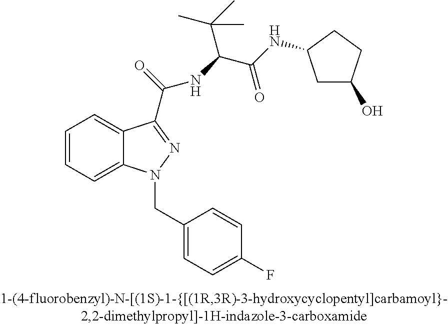 | ¹H NMR (400 MHz, DMSO-d₆) δ ppm 0.96 (s, 9H) 1.28-1.47 (m, 2H) 1.49-1.57 (m, 1H) 1.77 (d, J = 3.29 Hz, 1H) 1.84-1.91 (m, 1H) 1.94 (d, J = 12.08 Hz, 1H) 4.14-4.25 (m, 2H) 4.43-4.48 (m, 2H) 5.77 (s, 2H) 7.15 (t, J = 8.79 Hz, 2H) 7.26-7.35 (m, 3H) 7.45 (t, J = 7.69 Hz, 1H) 7.58 (d, J = 9.52 Hz, 1H) 7.78 (d, J = 8.42 Hz, 1H) 8.15-8.22 (m, 2H) | 467 |
| 290 | 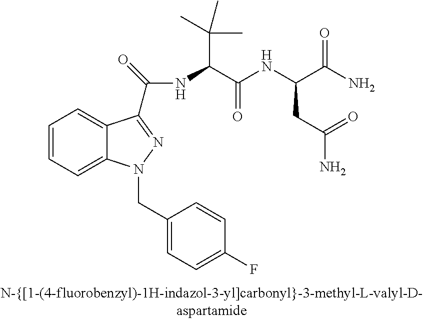 | ¹H NMR (400 MHz, DMSO-d₆) δ ppm 0.94-1.00 (m, 9H) 2.42 (d, J = 8.05 Hz, 1H) 2.56 (br. s., 1H) 4.50 (t, J = 8.97 Hz, 1H) 4.58 (d, J = 5.13 Hz, 1H) 5.76 (s, 2H) 6.82 (br. s., 1H) 7.00 (br. s., 1H) 7.15 (t, J = 8.60 Hz, 2H) 7.23-7.35 (m, 5H) 7.45 (t, J = 7.69 Hz, 1H) 7.62 (d, J = 8.79 Hz, 1H) 7.76 (d, J = 8.42 Hz, 1H) 8.16 (d, J = 8.05 Hz, 1H) 8.43 (d, J = 8.05 Hz, 1H) | 497 |
| 291 | 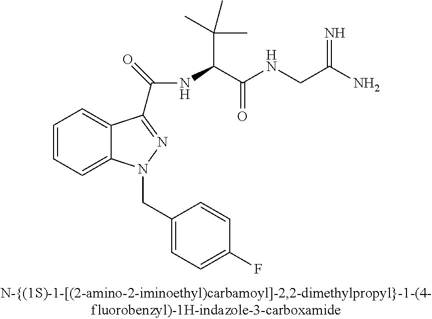 | ¹H NMR (400 MHz, DMSO-d₆) δ ppm 1.00 (s, 9H) 3.69 (d, J = 5.86 Hz, 2H) 4.53 (d, J = 9.15 Hz, 1H) 5.77 (s, 2H) 6.95 (br. s., 1H) 7.15 (t, J = 8.97 Hz, 2H) 7.27 (s, 1H) 7.31 (dt, J = 8.24, 5.58 Hz, 3H) 7.45 (t, J = 7.14 Hz, 1H) 7.62 (d, J = 9.52 Hz, 1H) 7.77 (d, J = 8.42 Hz, 1H) 8.17 (d, J = 8.42 Hz, 1H) 8.42 (t, J = 5.67 Hz, 1H) | 439 |
| 292 | 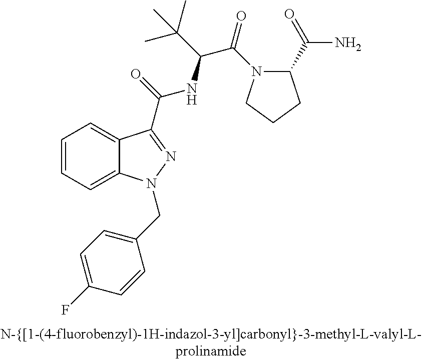 | ¹H NMR (400 MHz, DMSO-d6) δ ppm 0.95-1.02 (m, 9H) 1.73-1.90 (m, 2H) 1.91-2.13 (m, 2H) 3.62-3.81 (m, 2H) 4.29 (dd, J = 8.42, 5.49 Hz, 1H) 4.80 (d, J = 9.52 Hz, 1H) 5.76 (d, J = 4.39 Hz, 2H) 6.80 (br. s., 1H) 7.15 (t, J = 8.79 Hz, 2H) 7.26-7.36 (m, 3H) 7.46 (t, J = 7.32 Hz, 1H) 7.55 (d, J = 9.52 Hz, 1H) 7.79 (d, J = 8.05 Hz, 1H) 8.16 (d, J = 8.05 Hz, 1H) | 480 |
| 293 | 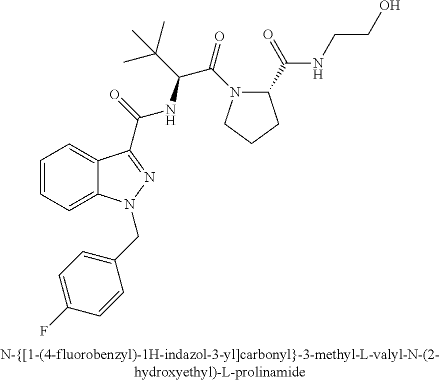 | ¹H NMR (400 MHz, DMSO-d6) δ ppm 1.00 (d, J = 20 Hz, 9H) 1.73-1.89 (m, 2H) 1.90-2.10 (m, 2H) 3.05-3.18 (m, 2H) 3.39 (q, J = 5.86 Hz, 2H) 3.61-3.81 (m, 2H) 4.27-4.36 (m, 1H) 4.55 (t, J = 5.49 Hz, 1H) 4.79 (d, J = 9.52 Hz, 1H) 5.76 (d, J = 5.13 Hz, 2H) 7.15 (t, J = 8.79 Hz, 2H) 7.25-7.36 (m, 3H) 7.46 (t, J = 7.69 Hz, 1H) 7.55 (d, J = 9.52 Hz, 1H) 7.79 (d, J = 8.79 Hz, 2H) 8.16 (d, J = 8.05 Hz, 1H) | 524 |
| 294 | 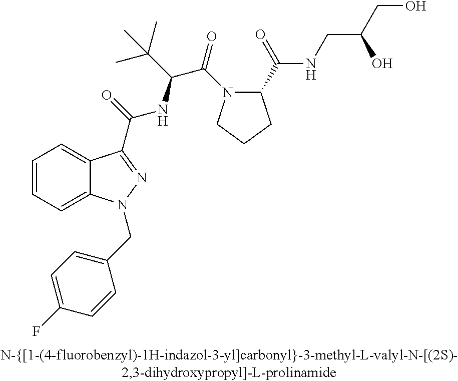 | ¹H NMR (400 MHz, DMSO-d6) δ ppm 0.94-1.08 (m, 9H) 1.71-1.90 (m, 2H) 1.90-2.11 (m, 2H) 2.96-3.07 (m, 1H) 3.14-3.25 (m, 1H) 3.31 (d, J = 5.86 Hz, 2H) 3.44-3.56 (m, 1H) 3.62-3.83 (m, 2H) 4.30-4.43 (m, 2H) 4.63 (d, J = 4.39 Hz, 1H) 4.79 (d, J = 9.52 Hz, 1H) 5.76 (d, J = 3.66 Hz, 2H) 7.15 (t, J = 8.79 Hz, 2H) 7.25-7.36 (m, 3H) 7.46 (t, J = 7.69 Hz, 1H) 7.55 (d, J = 8.79 Hz, 1H) 7.74-7.82 (m, 1H) 7.84 (t, J = 5.49 Hz, 1H) 8.16 (d, J = 8.05 Hz, 1H) | 554 |
| 295 | 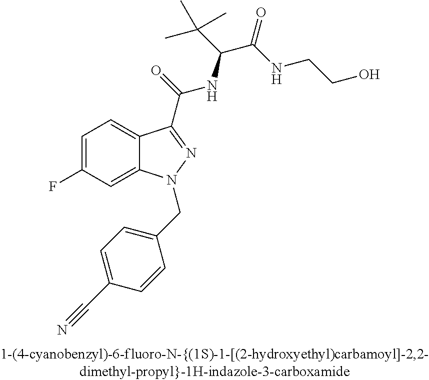 | ¹H NMR (400 MHz, DMSO-d6) δ ppm 8.16-8.31 (m, 2H) 7.80 (d, J = 8.05 Hz, 2H) 7.72 (d, J = 9.52 Hz, 1H) 7.58 (d, J = 9.88 Hz, 1H) 7.38 (d, J = 8.42 Hz, 2H) 7.16-7.26 (m, 1H) 5.86 (s, 2H) 4.61 (t, J = 5.31 Hz, 1H) 4.48 (d, J = 9.88 Hz, 1H) 3.41 (q, J = 5.98 Hz, 2H) 3.06-3.24 (m, 2H) 0.96 (s, 9H). | 452 |
| 296 | 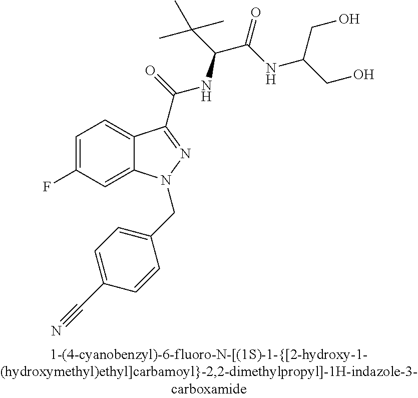 | ¹H NMR (400 MHz, DMSO-d6) δ ppm 8.20 (dd, J = 8.97, 5.31 Hz, 1H) 7.98 (d, J = 8.05 Hz, 1H) 7.80 (d, J = 8.05 Hz, 2H) 7.72 (d, J = 9.52 Hz, 1H) 7.59 (d, J = 9.88 Hz, 1H) 7.38 (d, J = 8.05 Hz, 2H) 7.16-7.24 (m, 1H) 5.87 (s, 2H) 4.52-4.61 (m, 3H) 3.71-3.83 (m, 1H) 3.36-3.50 (m, 4H) 0.96 (s, 9H). | 482 |
| 297 | 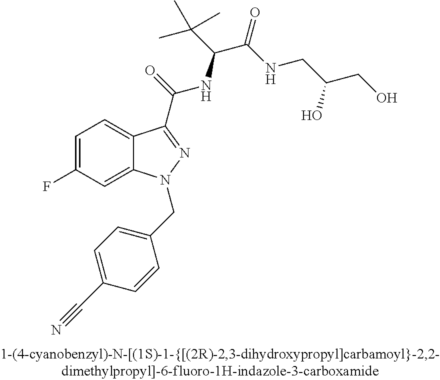 | ¹H NMR (400 MHz, DMSO-d6) δ ppm 8.15-8.27 (m, 2H) 7.80 (d, J = 8.05 Hz, 2H) 7.69-7.76 (m, 1H) 7.59 (d, J = 9.52 Hz, 1H) 7.39 (d, J = 8.05 Hz, 2H) 7.16-7.24 (m, 1H) 5.86 (s, 2H) 4.66 (d, J = 5.13 Hz, 1H) 4.43-4.57 (m, 2H) 3.45-3.53 (m, 1H) 3.30-3.36 (m, 1H) 3.14-3.23 (m, 1H) 3.03-3.12 (m, 1H) 0.96 (s, 9H). | 482 |
| 298 | 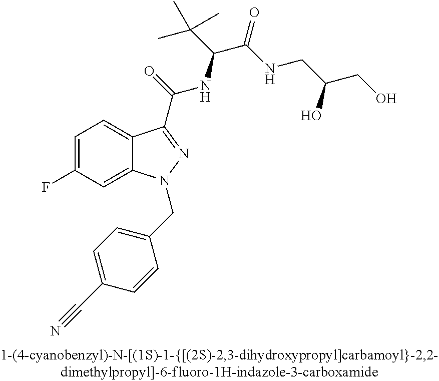 | ¹H NMR (400 MHz, DMSO-d6) δ ppm 8.16-8.26 (m, 2H) 7.80 (d, J = 8.42 Hz, 2H) 7.72 (d, J = 9.52 Hz, 1H) 7.60 (d, J = 9.52 Hz, 1H) 7.39 (d, J = 8.05 Hz, 2H) 7.19 (td, J = 9.15, 1.83 Hz, 1H) 5.86 (s, 2H) 4.64 (d, J = 5.13 Hz, 1H) 4.45-4.58 (m, 2H) 3.51 (dddd, J = 11.17, 5.49, 5.31, 5.13 Hz, 1H) 3.32 (br. s., 1H) 2.92-3.02 (m, 1H) 0.97 (s, 9H). | 482 |
| 299 | 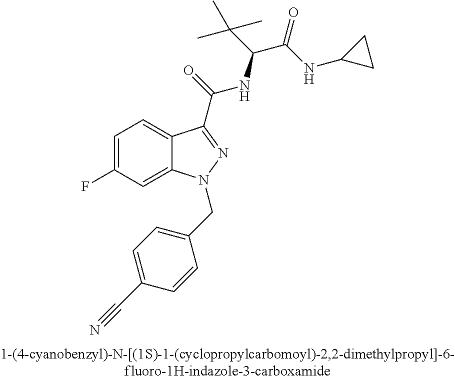 | ¹H NMR (400 MHz, DMSO-d6) δ ppm 8.30 (d, J = 4.03 Hz, 1H) 8.18 (dd, J = 8.79, 5.49 Hz, 1H) 7.80 (d, J = 8.05 Hz, 2H) 7.73 (d, J = 9.52 Hz, 1H) 7.55 (d, J = 9.52 Hz, 1H) 7.39 (d, J = 8.05 Hz, 2H) 7.19 (td, J = 9.24, 2.01 Hz, 1H) 5.87 (s, 2H) 4.39 (d, J = 9.88 Hz, 1H) 2.61-2.72 (m, J = 7.30, 7.30, 3.98, 3.98, 3.80 Hz, 1H) 0.94 (s, 9H) 0.58-0.67 (m, 2H) 0.35-0.46 (m, 2H). | 448 |
| 300 | 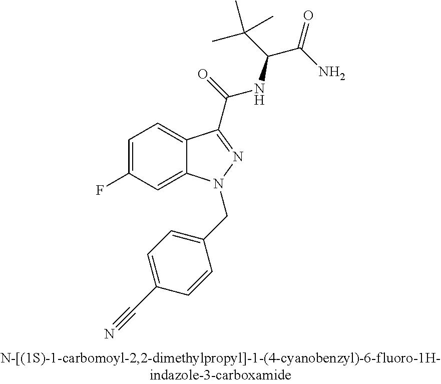 | ¹H NMR (400 MHz, DMSO-d6) δ ppm 8.19 (dd, J = 8.79, 5.49 Hz, 1H) 7.80 (d, J = 8.42 Hz, 2H) 7.66-7.75 (m, 2H) 7.57 (d, J = 9.52 Hz, 1H) 7.38 (d, J = 8.05 Hz, 2H) 7.15-7.24 (m, 2H) 5.86 (s, 2H) 4.44 (d, J = 9.52 Hz, 1H) 0.98 (s, 9H). | 408 |
| 301 | 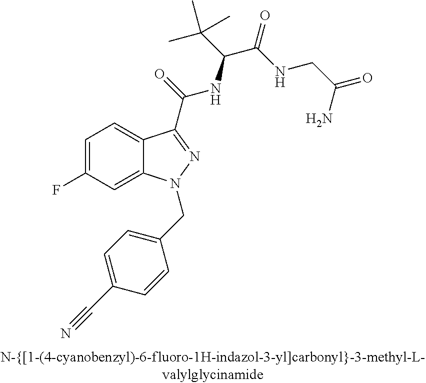 | ¹H NMR (400 MHz, DMSO-d6) δ ppm 8.42 (t, J = 5.67 Hz, 1H) 8.19 (dd, J = 8.79, 5.13 Hz, 1H) 7.80 (d, J = 8.05 Hz, 2H) 7.72 (d, J = 9.88 Hz, 1H) 7.61 (d, J = 9.15 Hz, 1H) 7.39 (d, J = 8.05 Hz, 2H) 7.16-7.29 (m, 2H) 6.94 (br. s., 1H) 5.86 (s, 2H) 4.52 (d, J = 9.52 Hz, 1H) 3.68 (d, J = 5.86 Hz, 2H) 0.98 (s, 9H). | 465 |
| 302 | 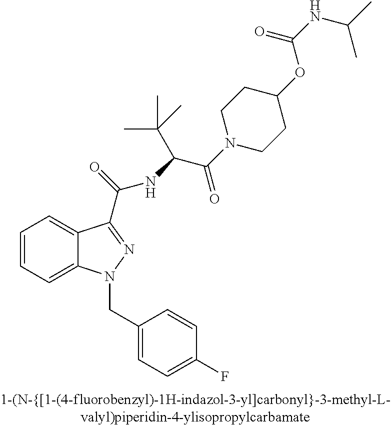 | ¹H NMR (400 MHz, DMSO-d₆) δ ppm 0.96-1.06 (m, 15H) 1.24-1.63 (m, 2H) 1.71-1.99 (m, 2H) 3.31-3.47 (m, 1H) 3.57 (dd, J = 12.26, 6.04 Hz, 2H) 4.02 (br. s., 1H) 4.73 (br. s., 1H) 5.05-5.12 (m, 1H) 5.77 (s, 2H) 6.84-7.03 (m, 1H) 7.15 (t, J = 8.24 Hz, 2H) 7.27-7.37 (m, 4H) 7.46 (t, J = 7.69 Hz, 1H) 7.61 (dd, J = 9.15, 3.29 Hz, 1H) 7.78 (dd, J = 8.42, 3.29 Hz, 1H) 8.16 (d, J = 8.05 Hz, 1H) | 552 |
| 303 | 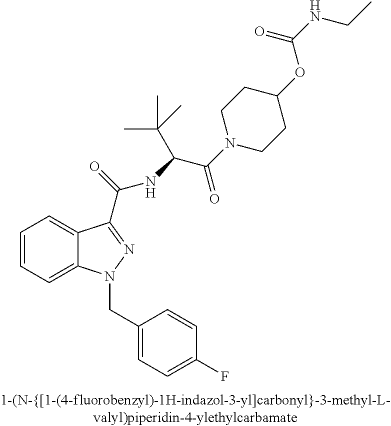 | ¹H NMR (400 MHz, DMSO-d₆) δ ppm 0.93-1.03 (m, 12H) 1.45 (br. s., 2H) 1.81 (br. s., 2H) 2.99 (dt, J = 13.00, 6.32 Hz, 2H) 3.07-3.22 (m, 1H) 3.31-3.49 (m, 1H) 3.50-3.62 (m, 1H) 3.83 (br. s., 1H) 4.02 (br. s., 1H) 4.69-4.78 (m, 1H) 5.09 (dd, J = 9.15, 6.59 Hz, 1H) 5.77 (s, 2H) 7.15 (t, J = 8.60 Hz, 2H) 7.27-7.37 (m, 3H) 7.46 (t, J = 7.69 Hz, 1H) 7.61 (dd, J = 9.34, 3.84 Hz, 1H) 7.78 (dd, J = 8.60, 2.75 Hz, 1H) 8.16 (d, J = 8.05 Hz, 1H) | 538 |
| 304 | 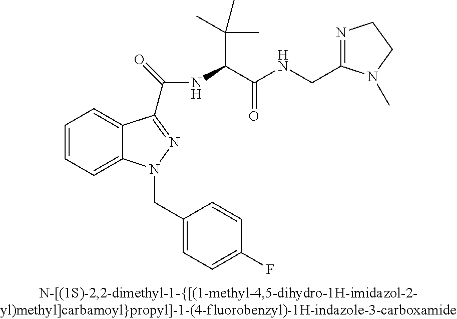 | ¹H NMR (400 MHz, DMSO-d₆) δ ppm 0.94-1.04 (m, 9H) 2.33 (s, 3H) 2.79-2.89 (m, 2H) 3.18-3.29 (m, 1H) 3.68-3.77 (m, 1H) 3.95-4.16 (m, 2H) 4.51-4.58 (m, 1H) 5.77 (s, 2H) 7.15 (t, J = 8.79 Hz, 2H) 7.26-7.36 (m, 3H) 7.45 (t, J = 7.69 Hz, 1H) 7.63 (dd, J = 11.72, 9.88 Hz, 1H) 7.78 (dd, J = 8.42, 2.56 Hz, 1H) 8.17 (d, J = 8.05 Hz, 1H) 8.30 (s, 1H) | 479 |
| 305 | 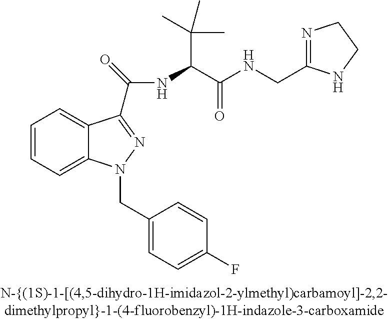 | ¹H NMR (400 MHz, DMSO-d₆) δ ppm 0.94-1.03 (m, 9H) 2.76 (s, 1H) 3.23 (d, J = 6.22 Hz, 1H) 3.65-3.80 (m, 1H) 3.91-4.10 (m, 3H) 4.47-4.59 (m, 1H) 5.77 (s, 2H) 7.15 (t, J = 8.79 Hz, 2H) 7.26-7.37 (m, 3H) 7.46 (t, J = 7.69 Hz, 1H) 7.65 (d, J = 9.52 Hz, 1H) 7.78 (d, J = 8.42 Hz, 1H) 8.17 (d, J = 8.42 Hz, 1H) 8.31 (s, 1H) 8.97 (br. s., 1H) | 465 |
| 306 | 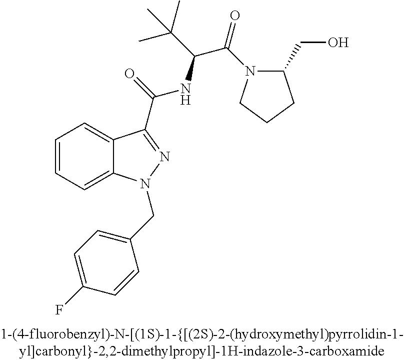 | ¹H NMR (400 MHz, DMSO-d6) δ ppm 0.93-1.06 (m, 9H) 1.74-1.92 (m, 2H) 3.29-3.39 (m, 1H) 3.48-3.56 (m, 1H) 3.56-3.73 (m, 2H) 4.02 (dd, J = 6.59, 2.93 Hz, 1H) 4.08-4.18 (m, 1H) 4.71 (t, J = 4.39 Hz, 1H) 4.77 (d, J = 9.52 Hz, 1H) 4.90 (d, J = 9.52 Hz, 1H) 5.77 (d, J = 2.93 Hz, 2H) 7.15 (t, J = 8.79 Hz, 2H) 7.24-7.36 (m, 3H) 7.42-7.49 (m, 1H) 7.50-7.61 (m, 1H) 7.79 (d, J = 8.79 Hz, 1H) 8.17 (t, J = 8.42 Hz, 1H) | 467 |
| 307 | 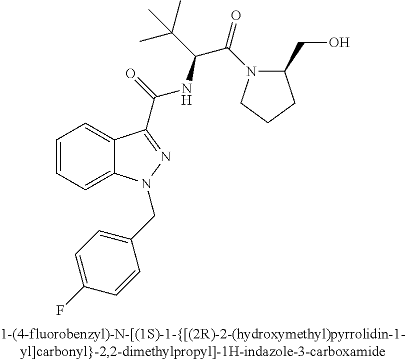 | ¹H NMR (400 MHz, DMSO-d6) δ ppm 0.96-1.08 (m, 9H) 1.77-2.03 (m, 3H) 3.29-3.35 (m, 2H) 3.45-3.54 (m, 1H) 3.55-3.73 (m, 2H) 3.97 (br. s., 1H) 4.66 (t, J = 5.86 Hz, 1H) 4.80 (d, J = 10.25 Hz, 1H) 5.74-5.80 (m, 2H) 7.15 (t, J = 8.79 Hz, 2H) 7.26-7.38 (m, 3H) 7.41-7.50 (m, 1H) 7.54 (d, J = 9.52 Hz, 1H) 7.75-7.83 (m, 1H) 8.17 (d, J = 8.79 Hz, 1H) | 467 |
| 308 | 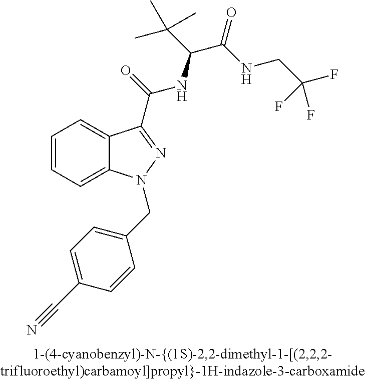 | ¹H NMR (400 MHz, DMSO-d6) δ ppm 8.95 (t, J = 6.22 Hz, 1H) 8.18 (d, J = 8.42 Hz, 1H) 7.73-7.84 (m, 3H) 7.62 (d, J = 9.52 Hz, 1H) 7.47 (t, J = 7.69 Hz, 1H) 7.28-7.40 (m, 3H) 5.91 (s, 2H) 4.60 (d, J = 9.52 Hz, 1H) 4.02-4.17 (m, 1H) 3.74-3.89 (m, 1H) 0.98 (s, 9H). | 472 |
| 309 | 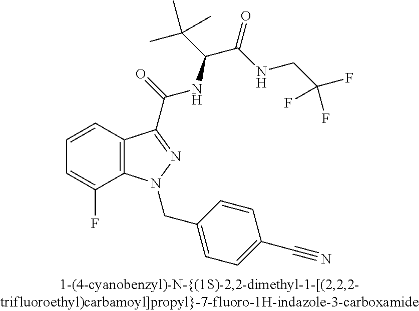 | ¹H NMR (400 MHz, DMSO-d6) δ ppm 8.95 (t, J = 6.22 Hz, 1H) 8.01 (d, J = 7.32 Hz, 1H) 7.80 (d, J = 8.05 Hz, 2H) 7.66 (d, J = 9.52 Hz, 1H) 7.23-7.39 (m, 4H) 5.93 (s, 2H) 4.61 (d, J = 9.52 Hz, 1H) 4.02-4.18 (m, 1H) 3.76-3.90 (m, 1H) 0.99 (s, 9H). | 490 |
| 310 | 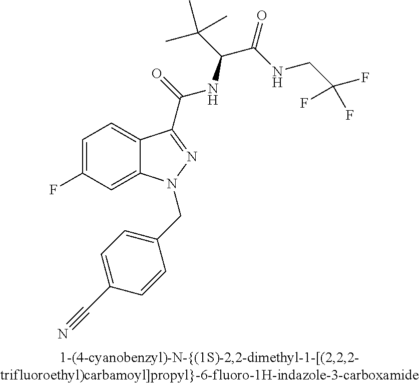 | ¹H NMR (400 MHz, DMSO-d6) δ ppm 8.94 (t, J = 6.22 Hz, 1H) 8.18 (dd, J = 8.97, 5.31 Hz, 1H) 7.80 (d, J = 8.05 Hz, 2H) 7.71-7.75 (m, 1H) 7.60 (d, J = 9.52 Hz, 1H) 7.39 (d, J = 8.05 Hz, 2H) 7.20 (td, J = 9.15, 1.83 Hz, 1H) 5.87 (s, 2H) 4.59 (d, J = 9.52 Hz, 1H) 4.01-4.17 (m, 1H) 3.74-3.90 (m, 1H) 0.97 (s, 9H). | 490 |
| 311 | 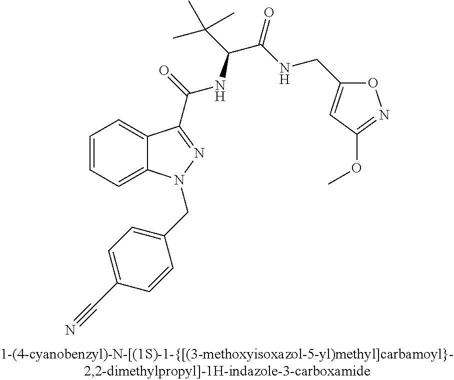 | ¹H NMR (400 MHz, DMSO-d6) δ ppm 8.89 (t, J = 5.67 Hz, 1H) 8.18 (d, J = 8.05 Hz, 1H) 7.72-7.87 (m, 3H) 7.61 (d, J = 9.52 Hz, 1H) 7.47 (t, J = 7.69 Hz, 1H) 7.27-7.43 (m, 3H) 6.03 (s, 1H) 5.91 (s, 2H) 4.52 (d, J = 9.88 Hz, 1H) 4.24-4.46 (m, 2H) 3.84 (s, 3H) 0.97 (s, 9H). | 501 |
| 312 | 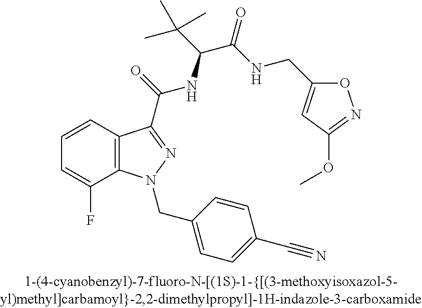 | ¹H NMR (400 MHz, DMSO-d6) δ ppm 8.90 (t, J = 5.67 Hz, 1H) 8.01 (d, J = 6.96 Hz, 1H) 7.80 (d, J = 8.42 Hz, 2H) 7.65 (d, J = 9.52 Hz, 1H) 7.24-7.38 (m, 4H) 6.03 (s, 1H) 5.93 (s, 2H) 4.53 (d, J = 9.88 Hz, 1H) 4.26-4.46 (m, 2H) 3.84 (s, 3H) 0.98 (s, 9H). | 519 |
| 313 | 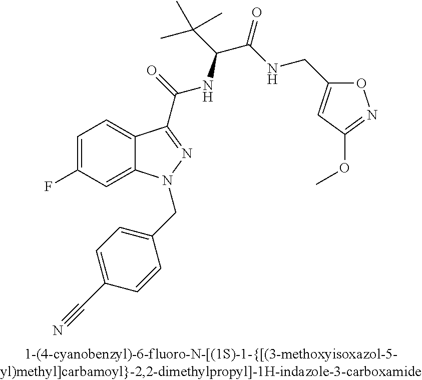 | ¹H NMR (400 MHz, DMSO-d6) δ ppm 8.89 (t, J = 5.49 Hz, 1H) 8.18 (dd, J = 8.97, 5.31 Hz, 1H) 7.80 (d, J = 8.42 Hz, 2H) 7.69-7.76 (m, 1H) 7.59 (d, J = 9.52 Hz, 1H) 7.39 (d, J = 8.42 Hz, 2H) 7.20 (td, J = 9.15, 2.20 Hz, 1H) 6.02 (s, 1H) 5.86 (s, 2H) 4.51 (d, J = 9.52 Hz, 1H) 4.25-4.45 (m, 2H) 3.84 (s, 3H) 0.96 (s, 9H). | 519 |
| 314 | 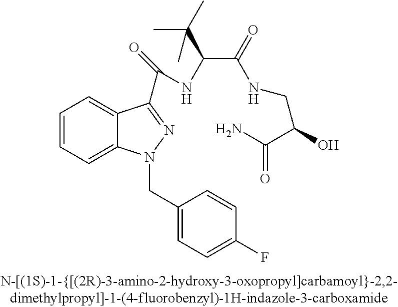 | ¹H NMR (400 MHz, DMSO-d₆) δ ppm 0.98 (s, 9H) 3.06-3.16 (m, 1H) 3.45-3.55 (m, 1H) 3.90 (d, J = 3.29 Hz, 1H) 4.56 (d, J = 9.52 Hz, 1H) 5.54 (d, J = 5.49 Hz, 1H) 5.70-5.81 (m, 2H) 7.12-7.21 (m, 4H) 7.25-7.36 (m, 3H) 7.45 (t, J = 7.69 Hz, 1H) 7.62 (d, J = 9.52 Hz, 1H) 7.77 (d, J = 8.42 Hz, 1H) 8.17 (d, J = 8.05 Hz, 1H) 8.25 (t, J = 5.49 Hz, 1H) | 470 |
| 315 | 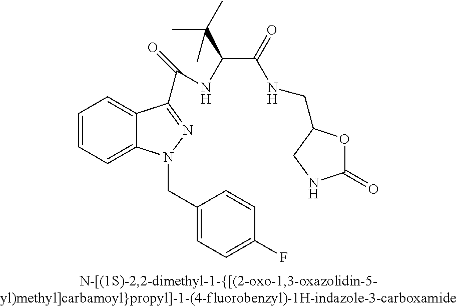 | ¹H NMR (400 MHz, DMSO-d₆) δ ppm 0.98 (s, 9H) 3.12-3.22 (m, 1H) 3.21-3.25 (m, 1H) 3.41-3.52 (m, 2H) 4.55 (d, J = 9.52 Hz, 1H) 4.57-4.66 (m, 1H) 5.77 (s, 2H) 7.15 (t, J = 8.79 Hz, 2H) 7.28-7.36 (m, 3H) 7.41-7.51 (m, 2H) 7.62 (dd, J = 9.70, 4.58 Hz, 1H) 7.77 (d, J = 8.42 Hz, 1H) 8.17 (d, J = 8.05 Hz, 1H) 8.58 (d, J = 7.69 Hz, 1H) | 482 |
| 316 | 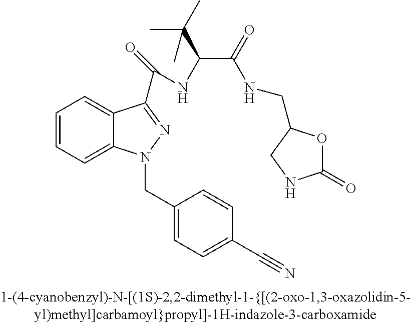 | ¹H NMR (400 MHz, DMSO-d₆) δ ppm 0.98 (s, 9H) 3.11-3.20 (m, 1H) 3.20-3.25 (m, 1H) 3.40-3.52 (m, 2H) 4.55 (d, J = 9.88 Hz, 1H) 4.60 (d, J = 2.20 Hz, 1H) 5.91 (s, 2H) 7.28-7.39 (m, 3H) 7.41-7.50 (m, 2H) 7.61 (dd, J = 9.52, 4.39 Hz, 1H) 7.75 (d, J = 8.42 Hz, 1H) 7.79 (d, J = 8.42 Hz, 2H) 8.19 (d, J = 8.05 Hz, 1H) 8.57 (d, J = 7.69 Hz, 1H) | 489 |
| 317 | 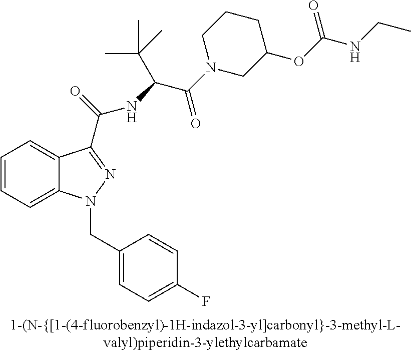 | ¹H NMR (400 MHz, DMSO-d₆) δ ppm 0.86-1.08 (m, 12H) 1.43 (br. s., 1H) 1.64 (br. s., 1H) 1.73 (br. s., 1H) 1.85 (br. s., 1H) 2.88-3.05 (m, 3H) 3.30 (br. s., 1H) 3.50 (br. s., 1H) 3.84 (br. s., 1H) 4.38-4.63 (m, 1H) 5.06 (d, J = 9.52 Hz, 1H) 5.77 (s, 2H) 6.79-6.98 (m, 1H) 7.15 (t, J = 8.79 Hz, 2H) 7.26-7.37 (m, 3H) 7.45 (t, J = 7.69 Hz, 1H) 7.64 (d, J = 9.52 Hz, 1H) 7.78 (d, J = 8.42 Hz, 1H) 8.16 (d, J = 8.05 Hz, 1H) | 538 |
| 318 | 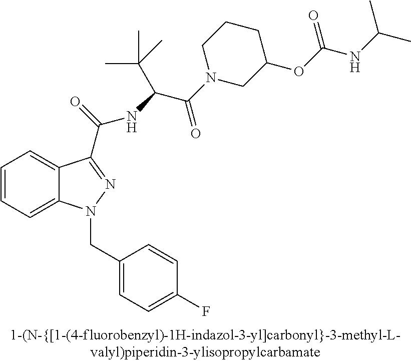 | ¹H NMR (400 MHz, DMSO-d₆) δ ppm 0.93-1.05 (m, 15H) 1.44 (br. s., 1H) 1.65 (br. s., 1H) 1.76 (br. s., 1H) 1.85 (br. s., 1H) 3.30 (br. s., 1H) 3.53 (br. s., 1H) 3.55-3.62 (m, 1H) 3.73 (br. s., 1H) 3.83 (br. s., 1H) 4.56 (br. s., 1H) 5.07 (d, J = 7.69 Hz, 1H) 5.77 (s, 2H) 7.15 (t, J = 8.97 Hz, 2H) 7.26-7.36 (m, 4H) 7.45 (t, J = 7.69 Hz, 1H) 7.64 (d, J = 9.52 Hz, 1H) 7.79 (d, J = 8.42 Hz, 1H) 8.17 (dd, J = 8.05, 3.30 Hz, 1H) | 552 |
| 319 | 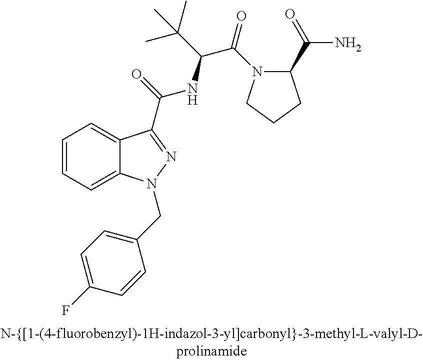 | ¹H NMR (400 MHz, DMSO-d6) δ ppm 0.95-1.07 (m, 9H) 1.77-1.99 (m, 3H) 2.00-2.11 (m, 1H) 3.63-3.82 (m, 2H) 4.18-4.28 (m, 1H) 4.79 (d, J = 8.79 Hz, 1H) 5.78 (s, 2H) 6.81 (br. s., 1H) 7.15 (t, J = 8.79 Hz, 2H) 7.23 (br. s., 1H) 7.26-7.37 (m, 3H) 7.42-7.49 (m, 1H) 7.55 (d, J = 8.79 Hz, 1H) 7.77 (d, J = 8.05 Hz, 1H) 8.17 (d, J = 8.05 Hz, 1H) | 480 |
| 320 | 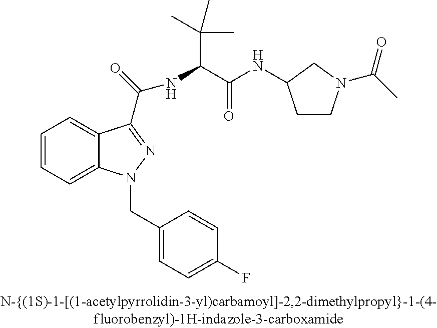 | ¹H NMR (400 MHz, DMSO-d₆) δ ppm 0.91-1.01 (m, 9H) 1.83-1.93 (m, 3H) 1.99 (dd, J = 12.81, 5.86 Hz, 1H) 2.04-2.13 (m, 1H) 3.19-3.25 (m, 1H) 3.30-3.35 (m, 1H) 3.39-3.50 (m, 1H) 3.64 (dd, J = 10.62, 5.86 Hz, 1H) 4.14-4.34 (m, 1H) 4.50 (dd, J = 9.70, 5.67 Hz, 1H) 5.77 (s, 2H) 7.15 (t, J = 8.97 Hz, 2H) 7.31 (dd, J = 10.98, 7.69 Hz, 3H) 7.46 (t, J = 7.69 Hz, 1H) 7.58 (d, J = 9.52 Hz, 1H) 7.78 (d, J = 8.42 Hz, 1H) 8.17 (d, J = 8.05 Hz, 1H) 8.43-8.56 (m, 1H) | 494 |
| 321 | 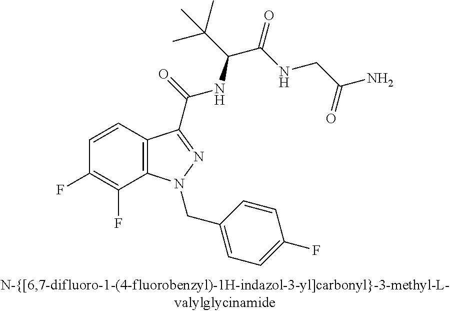 | ¹H NMR (400 MHz, DMSO-d6) δ ppm 8.43 (t, J = 5.67 Hz, 1H), 7.93-8.02 (m, 1H), 7.67 (d, J = 9.15 Hz, 1H), 7.31-7.41 (m, 1H), 7.22-7.31 (m, 3H), 7.12-7.22 (m, 2H), 6.95 (br. s., 1H), 5.78 (s, 2H), 4.53 (d, J = 9.52 Hz, 1H), 3.69 (d, J = 5.49 Hz, 2H), 0.99 (s, 9H) | 476 |
| 322 | 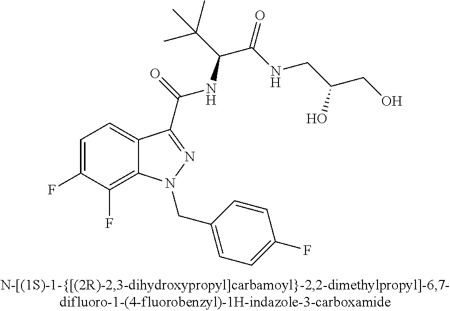 | ¹H NMR (400 MHz, DMSO-d6) δ ppm 8.18-8.26 (m, 1H), 7.94-8.02 (m, 1H), 7.65 (d, J = 9.52 Hz, 1H), 7.31-7.41 (m, 1H), 7.23-7.31 (m, 2H), 7.13-7.21 (m, 2H), 5.78 (s, 2H), 4.67 (d, J = 5.13 Hz, 1H), 4.53 (d, J = 9.88 Hz, 1H), 4.47 (t, J = 5.67 Hz, 1H), 3.45-3.55 (m, 1H), 3.31-3.34 (m, 1H), 3.13-3.24 (m, 1H), 3.03-3.12 (m, 1H), 0.97 (s, 9H) | 493 |
| 323 | 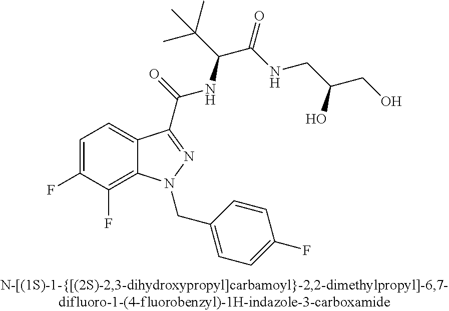 | ¹H NMR (400 MHz, DMSO-d6) δ ppm 8.21 (t, J = 5.49 Hz, 1H), 7.94-8.02 (m, 1H), 7.65 (d, J = 9.52 Hz, 1H), 7.31-7.41 (m, 1H), 7.23-7.31 (m, 2H), 7.13-7.20 (m, 2H), 5.78 (s, 2H), 4.65 (d, J = 5.13 Hz, 1H), 4.54 (d, J = 9.88 Hz, 1H), 4.48 (t, J = 5.86 Hz, 1H), 3.47-3.56 (m, 1H), 3.30-3.35 (m, 2H), 2.92-3.02 (m. 1H). 0.98 (s, 9H) | 493 |
| 324 | 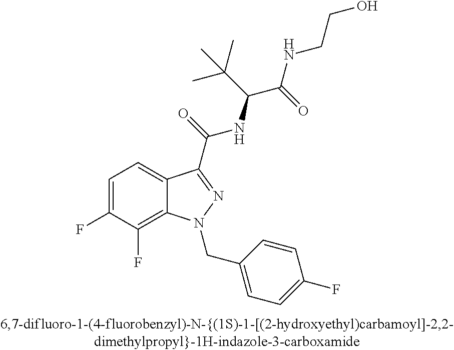 | ¹H NMR (400 MHz, DMSO-d6) δ ppm 8.25 (t, J = 5.49 Hz, 1H), 7.94-8.02 (m, 1H), 7.63 (d, J = 9.52 Hz, 1H), 7.31-7.40 (m, 1H), 7.23-7.31 (m, 2H), 7.13-7.21 (m, 2H), 5.78 (s, 2H), 4.61 (t, J = 5.31 Hz, 1H), 4.49 (d, J = 9.88 Hz, 1H), 3.42 (q, J = 5.61 Hz, 2H), 3.16-3.25 (m, 1H), 3.07-3.16 (m, 1H), 0.97 (s, 9H) | 463 |
| 325 | 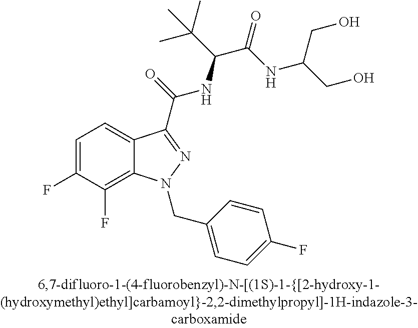 | ¹H NMR (400 MHz, DMSO-d6) δ ppm 7.94-8.03 (m, 2H), 7.65 (d, J = 9.52 Hz, 1H), 7.31-7.41 (m, 1H), 7.23-7.31 (m, 2H), 7.12-7.22 (m, 2H), 5.78 (s, 2H), 4.51-4.61 (m, 3H), 3.71-3.83 (m, 1H), 3.36-3.49 (m, 4H), 0.97 (s, 9H) | 493 |
| 326 | 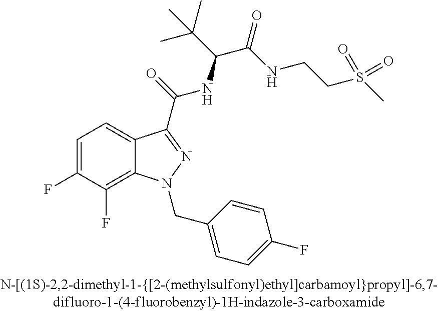 | ¹H NMR (400 MHz, DMSO-d6) δ ppm 8.51-8.58 (m, 1H), 7.93-8.01 (m, 1H), 7.64 (d, J = 9.52 Hz, 1H), 7.31-7.42 (m, 1H), 7.23-7.31 (m, 2H), 7.12-7.21 (m, 2H), 5.78 (s, 2H), 4.46 (d, J = 9.52 Hz, 1H), 3.42-3.59 (m, 2H), 3.20-3.35 (m, 2H), 2.99 (s, 3H), 0.98 (s, 9H) | 525 |
| 327 | 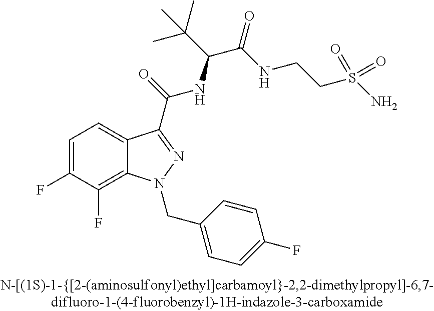 | ¹H NMR (400 MHz, DMSO-d6) δ ppm 8.42-8.51 (m, 1H), 7.93-8.02 (m, 1H), 7.63 (d, J = 9.89 Hz, 1H), 7.32-7.41 (m, 1H), 7.24-7.32 (m, 2H), 7.13-7.21 (m, 2H), 6.87 (s, 2H), 5.78 (s, 2H), 4.44 (d, J = 9.52 Hz, 1H), 3.36-3.59 (m, 2H), 3.03-3.21 (m, 2H), 0.94-1.02 (m, 9H) | 526 |
| 328 | 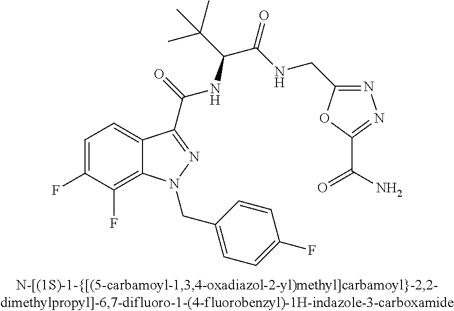 | ¹H NMR (400 MHz, DMSO-d6) δ ppm 9.06-9.15 (m, 1H), 8.51 (br. s., 1H), 8.15 (br. s., 1H), 7.92-8.01 (m, 1H), 7.66 (d, J = 9.88 Hz, 1H), 7.31-7.42 (m, 1H), 7.23-7.31 (m, 2H), 7.12-7.23 (m, 2H), 5.77 (s, 2H), 4.52-4.71 (m, 3H), 0.99 (s, 9H) | 544 |
| 329 | 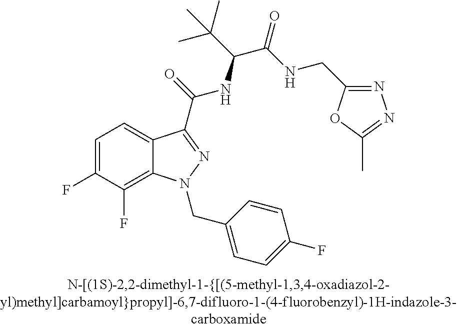 | ¹H NMR (400 MHz, DMSO-d6) δ ppm 8.96-9.05 (m, 1H), 7.93-8.01 (m, 1H), 7.65 (d, J = 9.88 Hz, 1H), 7.31-7.41 (m, 1H), 7.23-7.31 (m, 2H), 7.13-7.22 (m, 2H), 5.78 (s, 2H), 4.41-4.62 (m, 3H), 2.43 (s, 3H), 0.99 (s, 9H) | 515 |
| 330 | 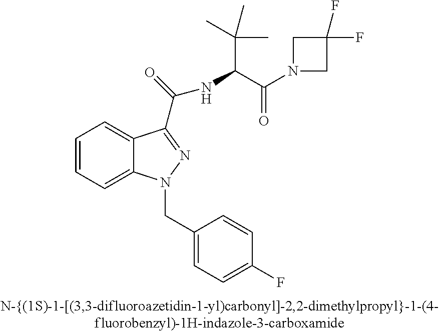 | ¹H NMR (400 MHz, DMSO-d₆) δ ppm 1.03 (s, 9H) 4.35 (d, J = 14.28 Hz, 2H) 4.43 (d, J = 9.15 Hz, 1H) 4.75 (br. s., 1H) 4.80 (br. s., 1H) 5.77 (d, J = 2.93 Hz, 2H) 7.15 (t, J = 8.79 Hz, 2H) 7.29-7.37 (m, 3H) 7.46 (t, J = 7.69 Hz, 1H) 7.59 (d, J = 9.15 Hz, 1H) 7.79 (d, J = 8.42 Hz, 1H) 8.16 (d, J = 8.05 Hz, 1H) | 459 |
| 331 | 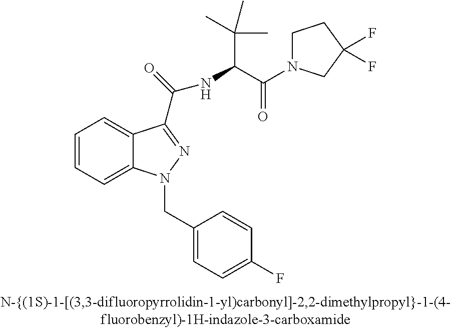 | ¹H NMR (400 MHz, DMSO-d₆) δ ppm 1.03 (s, 9H) 2.41 (br. s., 2H) 3.44-3.69 (m, 1H) 3.75 (d, J = 15.01 Hz, 1H) 3.91 (s, 1H) 4.15 (d, J = 5.49 Hz, 1H) 4.61-4.84 (m, 1H) 5.77 (d, J = 2.56 Hz, 2H) 7.15 (t, J = 8.79 Hz, 2H) 7.27-7.36 (m, 3H) 7.46 (t, J = 7.69 Hz, 1H) 7.56 (d, J = 9.52 Hz, 1H) 7.79 (d, J = 8.42 Hz, 1H) 8.16 (d, J = 8.42 Hz, 1H) | 473 |
| 332 | 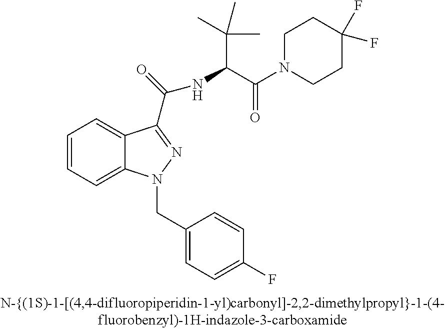 | ¹H NMR (400 MHz, DMSO-d₆) δ ppm 0.97-1.05 (m, 9H) 1.92 (br. s., 2H) 2.02 (d, J = 12.81 Hz, 2H) 3.44 (br. s., 1H) 3.66 (d, J = 6.96 Hz, 1H) 3.78-3.89 (m, 1H) 3.92 (br. s., 1H) 5.08 (d, J = 9.52 Hz, 1H) 5.77 (s, 2H) 7.15 (t, J = 8.79 Hz, 2H) 7.27-7.36 (m, 3H) 7.46 (t, J = 7.69 Hz, 1H) 7.65 (d, J = 9.15 Hz, 1H) 7.79 (d, J = 8.42 Hz, 1H) 8.16 (d, J = 8.05 Hz, 1H) | 487 |
| 333 | 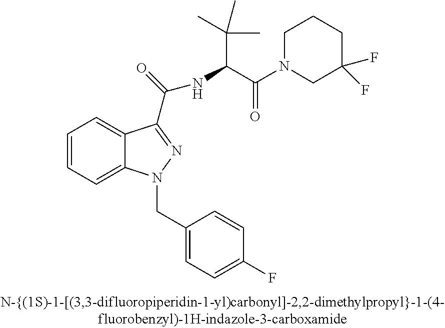 | ¹H NMR (400 MHz, DMSO-d₆) δ ppm 0.97-1.03 (m, 9H) 1.70-1.77 (m, 2H) 2.07 (td, J = 13.82, 7.51 Hz, 2H) 3.68 (d, J = 6.22 Hz, 1H) 3.78 (t, J = 13.00 Hz, 2H) 3.92 (t, J = 12.26 Hz, 1H) 5.10 (d, J = 9.88 Hz, 1H) 5.77 (s, 2H) 7.15 (t, J = 8.79 Hz, 2H) 7.27-7.37 (m, 3H) 7.46 (t, J = 7.69 Hz, 1H) 7.59 (d, J = 9.88 Hz, 1H) 7.78 (d, J = 8.42 Hz, 1H) 8.16 (d, J = 8.05 Hz. 1H) | 487 |
| 334 | 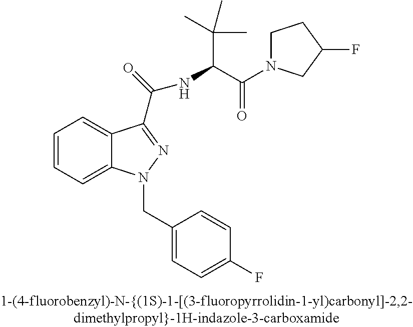 | ¹H NMR (400 MHz, DMSO-d₆) δ ppm 1.02 (d, J = 2.56 Hz, 9H) 2.11 (d, J = 2.93 Hz, 1H) 2.22 (br. s., 1H) 3.54 (br. s., 1H) 3.68 (d, J = 8.05 Hz, 1H) 3.71 (br. s., 1H) 3.86-3.97 (m, 1H) 4.70-4.88 (m, 1H) 5.20-5.41 (m, 1H) 5.77 (s, 2H) 7.15 (t, J = 8.60 Hz, 2H) 7.27-7.36 (m, 3H) 7.46 (t, J = 7.69 Hz, 1H) 7.51-7.58 (m, 1H) 7.78 (dd, J = 8.42, 4.76 Hz, 1H) 8.16 (d, J = 8.05 Hz, 1H) | 455 |
| 335 | 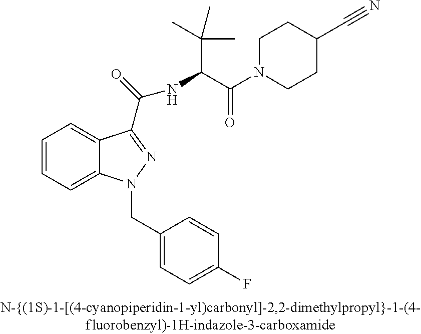 | ¹H NMR (400 MHz, DMSO-d₆) δ ppm 1.00 (d, J = 8.05 Hz, 9H) 1.53-1.80 (m, 2H) 1.80-2.02 (m, 2H) 3.08-3.19 (m, 2H) 3.36-3.48 (m, 1H) 3.57-3.88 (m, 1H) 3.96 (dd, J = 13.18, 3.66 Hz, 1H) 5.06 (d, J = 9.15 Hz, 1H) 5.77 (s, 2H) 7.15 (t, J = 8.79 Hz, 2H) 7.28-7.36 (m, 3H) 7.46 (t, J = 7.69 Hz, 1H) 7.57-7.68 (m, 1H) 7.78 (d, J = 8.42 Hz, 1H) 8.16 (t, J = 7.14 Hz, 1H) | 476 |
| 336 | 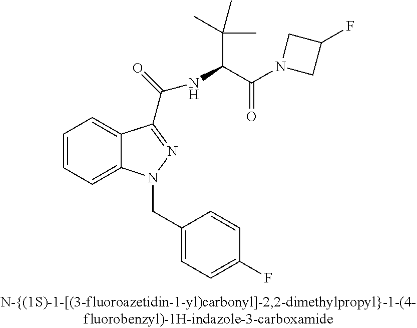 | ¹H NMR (400 MHz, DMSO-d₆) δ ppm 1.01 (s, 9H) 3.87-3.98 (m, 1H) 4.29 (br. s., 1H) 4.31 (d, J = 3.66 Hz, 1H) 4.44 (d, J = 9.52 Hz, 1H) 4.62 (br. s., 1H) 5.27-5.54 (m, 1H) 5.77 (br. s., 2H) 7.15 (t, J = 8.97 Hz, 2H) 7.27-7.36 (m, 3H) 7.46 (t, J = 7.69 Hz, 1H) 7.54 (t, J = 9.34 Hz, 1H) 7.79 (d, J = 8.42 Hz, 1H) 8.16 (d, J = 7.69 Hz, 1H) | 441 |
| 337 | 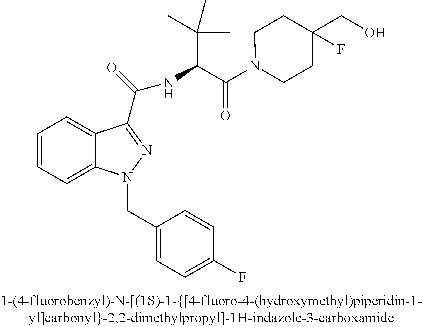 | ¹H NMR (400 MHz, DMSO-d₆) δ ppm 1.00 (d, J = 5.13 Hz, 9H) 1.52-1.64 (m, 1H) 1.69 (d, J = 2.93 Hz, 1H) 1.73 (br. s., 1H) 1.84 (br. s., 1H) 2.95 (d, J = 12.81 Hz, 1H) 3.33-3.45 (m, 2H) 4.09 (br. s., 1H) 4.18 (d, J = 14.28 Hz, 1H) 4.29 (d, J = 12.45 Hz, 1H) 4.92 (t, J = 5.86 Hz, 1H) 5.10 (dd, J = 9.15, 5.13 Hz, 1H) 5.77 (s, 2H) 7.15 (t, J = 8.79 Hz, 2H) 7.26-7.36 (m, 3H) 7.46 (t, J = 7.69 Hz, 1H) 7.63 (t, J = 10.07 Hz, 1H) 7.79 (d, J = 8.42 Hz, 1H) 8.17 (d, J = 8.42 Hz, 1H) | 499 |
| 338 | 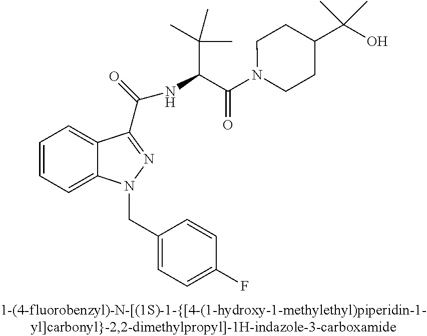 | ¹H NMR (400 MHz, DMSO-d₆) δ ppm 0.98 (s, 9H) 1.01 (s, 3H) 1.04 (s, 3H) 1.09 (br. s., 1H) 1.21 (dd, J = 12.08, 3.66 Hz, 1H) 1.43 (d, J = 9.52 Hz, 1H) 1.65-1.73 (m, 1H) 1.79 (d, J = 9.52 Hz, 1H) 2.45 (br. s., 1H) 2.96-3.06 (m, 1H) 4.03-4.15 (m, 1H) 4.26 (br. s., 1H) 4.54 (d, J = 12.81 Hz, 1H) 5.09 (t, J = 8.42 Hz, 1H) 5.77 (s, 2H) 7.15 (t, J = 8.79 Hz, 2H) 7.26-7.36 (m, 3H) 7.45 (t, J = 7.69 Hz, 1H) 7.58-7.68 (m, 1H) 7.78 (d, J = 8.79 Hz, 1H) 8.17 (d, J = 8.42 Hz, 1H) | 509 |
| 339 | 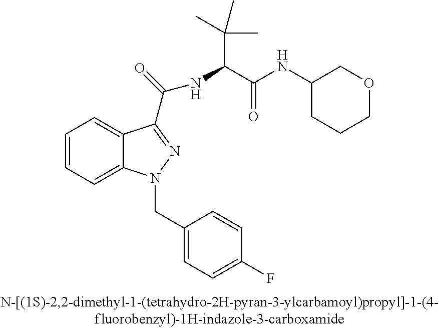 | ¹H NMR (400 MHz, DMSO-d₆) δ ppm 0.97 (d, J = 4.39 Hz, 9H) 1.42-1.54 (m, 2H) 1.62-1.72 (m, 1H) 1.82 (br. s., 1H) 3.07-3.16 (m, 1H) 3.34 (d, J = 11.72 Hz, 1H) 3.62-3.74 (m, 3H) 4.54 (dd, J = 9.70, 5.67 Hz, 1H) 5.77 (s, 2H) 7.15 (t, J = 8.79 Hz, 2H) 7.31 (dt, J = 8.05, 5.49 Hz, 3H) 7.45 (t, J = 7.51 Hz, 1H) 7.58 (dd, J = 9.70, 3.11 Hz, 1H) 7.78 (d, J = 8.79 Hz, 1H) 8.17 (d, J = 8.42 Hz, 1H) 8.19-8.29 (m, 1H) | 467 |
| 340 | 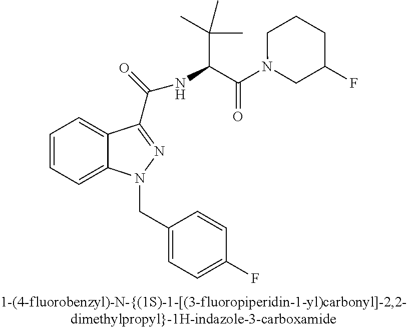 | ¹H NMR (400 MHz, DMSO-d₆) δ ppm 0.92-1.05 (m, 9H) 1.57 (br. s., 1H) 1.73-1.92 (m, 2H) 3.34 (br. s., 1H) 3.39 (d, J = 14.28 Hz, 1H) 3.89 (br. s., 1H) 4.11-4.20 (m, 1H) 4.68 (br. s., 1H) 4.79 (br. s., 1H) 5.07-5.14 (m, 1H) 5.77 (s, 2H) 7.15 (t, J = 8.79 Hz, 2H) 7.28-7.36 (m, 3H) 7.46 (t, J = 7.69 Hz, 1H) 7.58-7.67 (m, 1H) 7.78 (dd, J = 8.42, 3.66 Hz, 1H) 8.17 (dd, J = 8.05, 2.93 Hz, 1H) | 467 |
| 341 | 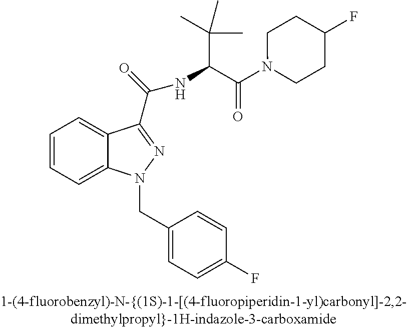 | ¹H NMR (400 MHz, DMSO-d₆) δ ppm 1.00 (s, 9H) 1.54-2.02 (m, 4H) 3.38-3.54 (m, 1H) 3.53-3.90 (m, 3H) 4.75-4.97 (m, 1H) 5.03-5.14 (m, 1H) 5.77 (s, 2H) 7.15 (t, J = 8.79 Hz, 2H) 7.25-7.38 (m, 3H) 7.46 (t, J = 7.69 Hz, 1H) 7.62 (dd, J = 9.34, 2.38 Hz, 1H) 7.78 (d, J = 8.42 Hz, 1H) 8.16 (d, J = 8.05 Hz, 1H) | 469 |
| 342 | 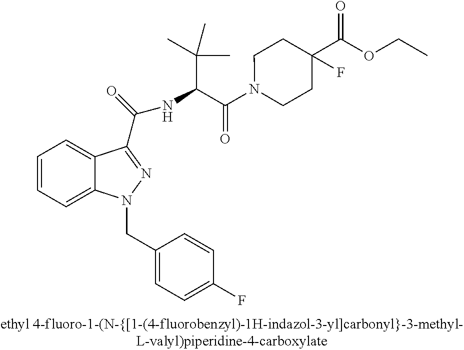 | ¹H NMR (400 MHz, DMSO-d₆) δ ppm 1.01 (d, J = 7.32 Hz, 9H) 1.09-1.27 (m, 3H) 1.71-2.10 (m, 4H) 2.86-3.05 (m, 1H) 3.32-3.49 (m, 1H) 4.12 (q, J = 7.20 Hz, 2H) 4.17-4.29 (m, 1H) 4.35 (br. s., 1H) 5.09 (d, J = 9.52 Hz, 1H) 5.77 (s, 2H) 7.15 (t, J = 8.60 Hz, 2H) 7.24-7.37 (m, 3H) 7.46 (t, J = 7.69 Hz, 1H) 7.63 (dd, J = 17.21, 9.15 Hz, 1H) 7.78 (d, J = 8.42 Hz, 1H) 8.16 (d, J = 8.05 Hz, 1H) | 541 |
| 343 | 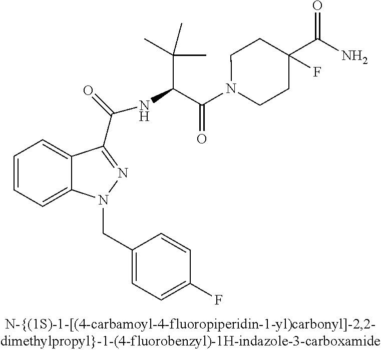 | ¹H NMR (400 MHz, DMSO-d₆) δ ppm 1.01 (d, J = 8.42 Hz, 9H) 1.89 (br. s., 3H) 1.98 (br. s., 1H) 2.90 (t, J = 12.26 Hz, 1H) 3.34 (br. s., 1H) 4.21 (br. s., 1H) 4.38 (br. s., 1H) 5.10 (dd, J = 9.34, 5.31 Hz, 1H) 5.77 (s, 2H) 7.15 (t, J = 8.79 Hz, 2H) 7.27-7.39 (m, 4H) 7.41-7.49 (m, 1H) 7.52-7.70 (m, 2H) 7.77 (dd, J = 8.24, 4.58 Hz, 1H) 8.17 (d, J = 8.42 Hz, 1H) | 512 |
| 344 | 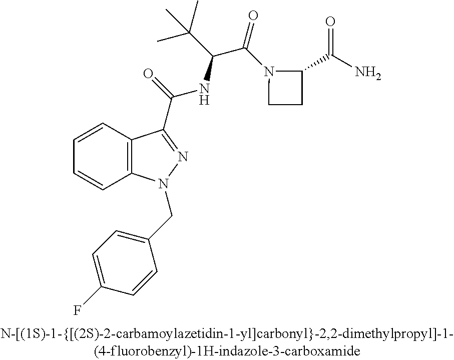 | ¹H NMR (400 MHz, DMSO-d6) δ ppm 0.92-1.09 (m, 9H) 2.14 (br. s., 1H) 2.40 (d, J = 8.79 Hz, 1H) 4.24 (t, J = 7.69 Hz, 1H) 4.44 (d, J = 9.52 Hz, 1H) 4.62 (dd, J = 8.79, 5.86 Hz, 1H) 5.77 (d, J = 3.66 Hz, 2H) 7.07 (br. s., 1H) 7.15 (t, J = 8.79 Hz, 2H) 7.23-7.36 (m, 4H) 7.41-7.56 (m, 2H) 7.79 (d, J = 8.05 Hz, 1H) 8.17 (d, J = 8.05 Hz, 1H) | 466 |
| 345 | 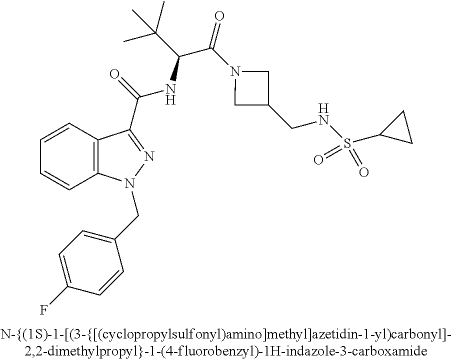 | ¹H NMR (400 MHz, DMSO-d6) δ ppm 0.83-0.96 (m, 4H) 0.99 (s, 9H) 2.53-2.61 (m, 1H) 2.71 (d, J = 8.79 Hz, 2H) 3.11-3.23 (m, 2H) 3.54-3.69 (m, 2H) 3.90 (dd, J = 17.21, 8.42 Hz, 1H) 3.94-4.05 (m, 1H) 4.25-4.36 (m, 1H) 4.42 (d, J = 9.52 Hz, 1H) 5.77 (d, J = 5.13 Hz, 2H) 7.16 (t, J = 8.79 Hz, 2H) 7.23-7.36 (m, 3H) 7.42-7.49 (m, 1H) 7.52 (d, J = 9.52 Hz, 1H) 7.80 (d, J = 8.79 Hz, 1H) 8.16 (d, J = 8.05 Hz, 1H) | 556 |
| 346 | 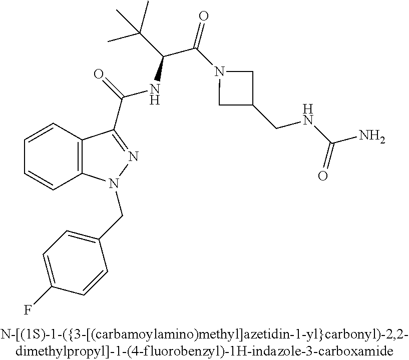 | ¹H NMR (400 MHz, DMSO-d6) δ ppm 0.98 (s, 9H) 2.56-2.73 (m, 2H) 3.07-3.24 (m, 2H) 3.50-3.64 (m, 1H) 3.86 (dd, J = 16.11, 8.79 Hz, 1H) 3.91-3.98 (m, 1H) 4.20-4.31 (m, 1H) 4.42 (dd, J = 9.52, 4.39 Hz, 1H) 5.45 (d, J = 14.64 Hz, 2H) 5.77 (d, J = 4.39 Hz, 2H) 6.11-6.25 (m, 1H) 7.16 (t, J = 8.79 Hz, 2H) 7.26-7.36 (m, 3H) 7.42-7.49 (m, 1H) 7.51 (d, J = 9.52 Hz, 1H) 7.80 (d, J = 8.79 Hz, 1H) 8.16 (dd, J = 8.42, 4.03 Hz, 1H) | 495 |
| 347 | 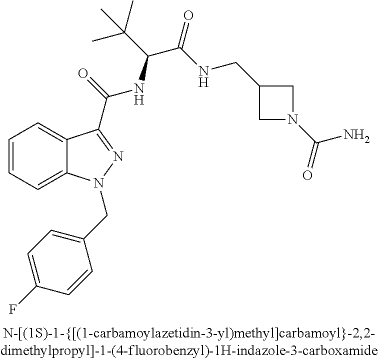 | ¹H NMR (400 MHz, DMSO-d6) δ ppm 0.87-1.00 (m, 9H) 2.09 (s, 1H) 3.07-3.17 (m, 2H) 3.17-3.27 (m, 2H) 3.43 (dd, J = 13.54, 8.42 Hz, 2H) 3.71-3.81 (m, 1H) 4.16 (d, J = 10.98 Hz, 1H) 4.47 (d, J = 9.52 Hz, 1H) 5.72-5.82 (m, 2H) 7.09-7.20 (m, 2H) 7.24-7.36 (m, 3H) 7.40-7.50 (m, 1H) 7.60 (d, J = 9.52 Hz, 1H) 7.75-7.83 (m, 1H) 8.16 (d, J = 8.05 Hz, 1H) 8.42-8.53 (m, 1H) | 495 |
| 348 | 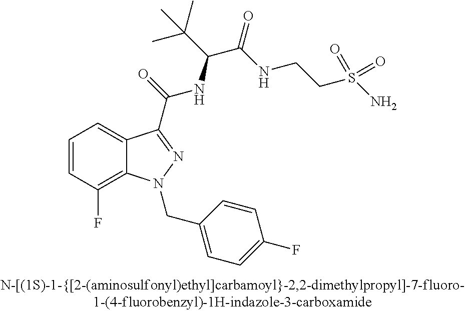 | ¹H NMR (400 MHz, DMSO-d6) δ ppm 8.50-8.55 (m, 1H), 7.95-8.01 (m, 1H), 7.61-7.67 (m, 1H), 7.21-7.35 (m, 4H), 7.12-7.21 (m, 2H), 6.92 (br. s., 2H), 5.80 (s, 2H), 4.44 (d, J = 9.88 Hz, 1H), 3.36-3.58 (m, 2H), 3.04-3.20 (m, 2H), 0.97 (s, 9H) | 508 |
| 349 | 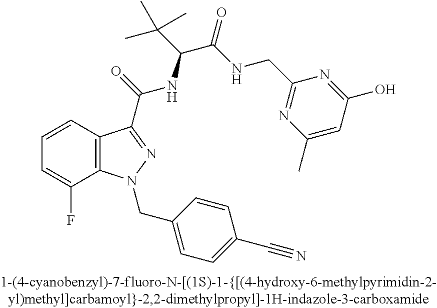 | ¹H NMR (400 MHz, DMSO-d6) δ ppm 12.29 (br. s., 1H) 8.78 (t, J = 5.67 Hz, 1H) 8.02 (d, J = 7.32 Hz, 1H) 7.80 (d, J = 8.42 Hz, 2H) 7.69 (d, J = 9.52 Hz, 1H) 7.23-7.37 (m, 4H) 6.03 (s, 1H) 5.93 (s, 2H) 4.57 (d, J = 9.15 Hz, 1H) 4.08-4.24 (m, 2H) 2.07 (s, 3H) 1.01 (s, 9H). | 530 |
| 350 | 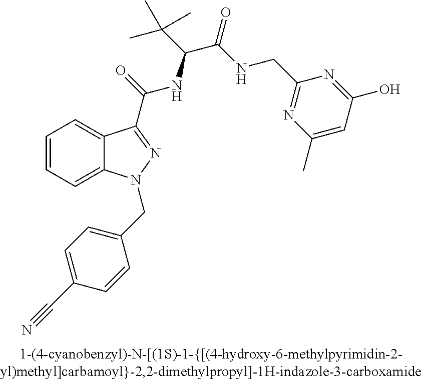 | ¹H NMR (400 MHz, DMSO-d6) δ d ppm 12.29 (br. s., 1H) 8.77 (t, J = 5.67 Hz, 1H) 8.19 (d, J = 8.05 Hz, 1H) 7.74-7.86 (m, 3H) 7.63 (d, J = 9.52 Hz, 1H) 7.46 (t, J = 7.69 Hz, 1H) 7.27-7.40 (m, 3H) 6.03 (s, 1H) 5.91 (s, 2H) 4.56 (d, J = 9.88 Hz, 1H) 4.08-4.24 (m, 2H) 2.07 (s, 3H) 1.00 (s, 9H). | 512 |
| 351 | 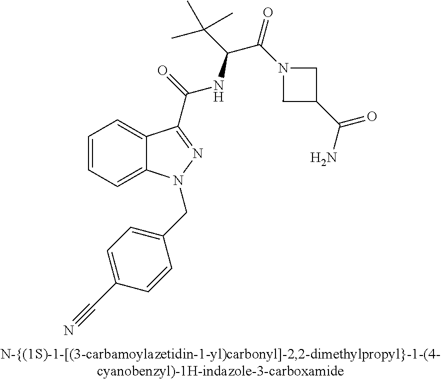 | ¹H NMR (400 MHz, DMSO-d6) δ ppm 8.12-8.23 (m, 1H) 7.72-7.84 (m, 3H) 7.43-7.56 (m, 3H) 7.26-7.38 (m, 3H) 7.08 (br. s., 1H) 5.86-5.99 (m, 2H) 4.21-4.51 (m, 3H) 3.92-4.06 (m, 1H) 3.77-3.89 (m, 1H) 3.23-3.30 (m, 1H) 0.98 (s, 9H). | 473 |
| 352 | 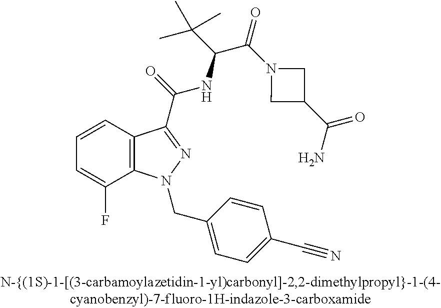 | ¹H NMR (400 MHz, DMSO-d6) δ ppm 8.00 (d, J = 7.69 Hz, 1H) 7.80 (dd, J = 8.42, 4.39 Hz, 2H) 7.60 (d, J = 8.79 Hz, 1H) 7.50 (br. s., 1H) 7.24-7.36 (m, 4H) 7.08 (br. s., 1H) 5.93 (s, 2H) 4.33-4.52 (m, 2H) 4.27 (t, J = 6.22 Hz, 1H) 3.94-4.06 (m, 1H) 3.79-3.90 (m, 1H) 3.22-3.30 (m, 1H) 0.99 (s, 9H). | 491 |
| 353 | 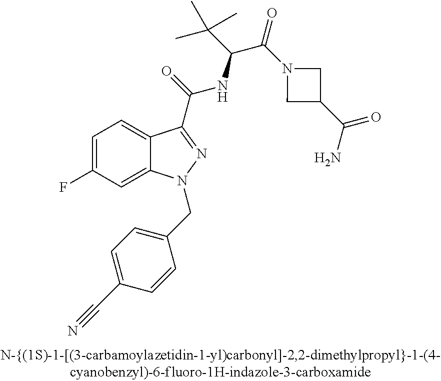 | ¹H NMR (400 MHz, DMSO-d6) δ ppm 8.14-8.22 (m, 1H) 7.70-7.85 (m, 3H) 7.47-7.59 (m, 2H) 7.37 (d, J = 8.05 Hz, 2H) 7.16-7.26 (m, 1H) 7.08 (br. s., 1H) 5.80-5.96 (m, 2H) 4.20-4.50 (m, 3H) 3.93-4.07 (m, 1H) 3.78-3.89 (m, 1H) 3.23-3.31 (m, 1H) 0.97 (s, 9H). | 491 |
| 354 | 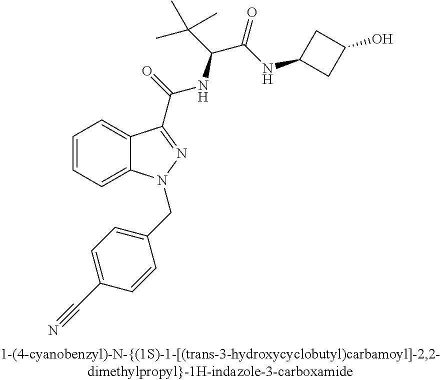 | ¹H NMR (400 MHz, DMSO-d6) δ ppm 8.43-8.58 (m, 1H) 8.18 (d, J = 8.05 Hz, 1H) 7.73-7.84 (m, 3H) 7.56 (d, J = 9.88 Hz, 1H) 7.46 (t, J = 7.69 Hz, 1H) 7.27-7.38 (m, 3H) 5.92 (s, 2H) 5.01-5.12 (m, 1H) 4.45 (dd, J = 9.70, 5.67 Hz, 1H) 4.10-4.30 (m, 1H) 3.61-3.83 (m, 1H) 2.40-2.47 (m, 1H) 2.02-2.17 (m, 2H) 1.63-1.80 (m, 1H) 0.95 (d, J = 3.29 Hz, 9H). | 460 |
| 355 | 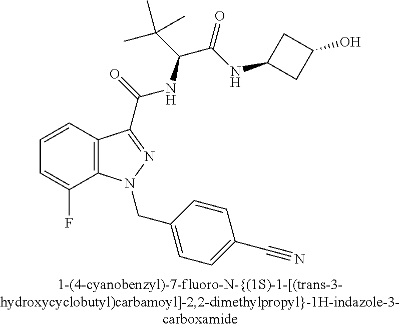 | ¹H NMR (400 MHz, DMSO-d6) δ ppm 8.45-8.58 (m, 1H) 8.01 (d, J = 7.32 Hz, 1H) 7.80 (d, J = 8.42 Hz, 2H) 7.60 (d, J = 9.88 Hz, 1H) 7.20-7.36 (m, 4H) 5.93 (s, 2H) 5.03-5.14 (m, 1H) 4.46 (dd, J = 9.52, 5.49 Hz, 1H) 4.07-4.29 (m, 1H) 3.59-3.83 (m, 1H) 2.40-2.47 (m, 1H) 2.01-2.16 (m, 2H) 1.63-1.80 (m, 1H) 0.95 (d, J = 3.29 Hz, 9H). | 478 |
| 356 | 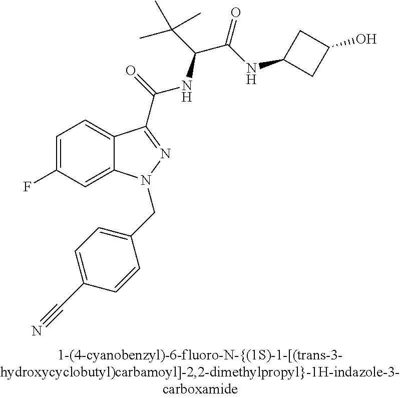 | ¹H NMR (400 MHz, DMSO-d6) δ ppm 8.43-8.59 (m, 1H) 8.18 (dd, J = 8.97, 5.31 Hz, 1H) 7.70-7.87 (m, 3H) 7.54 (d, J = 9.52 Hz, 1H) 7.37 (d, J = 8.05 Hz, 2H) 7.20 (td, J = 9.15, 2.20 Hz, 1H) 5.87 (s, 2H) 5.00-5.14 (m, 1H) 4.43 (dd, J = 9.70, 5.67 Hz, 1H) 4.08-4.29 (m, 1H) 3.59-3.84 (m, 1H) 2.39-2.47 (m, 1H) 2.07 (t, J = 6.04 Hz, 2H) 1.64-1.80 (m, 1H) 0.94 (d, J = 3.29 Hz, 9H). | 478 |
| 357 | 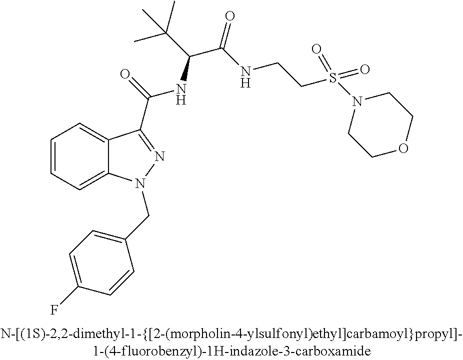 | ¹H NMR (400 MHz, DMSO-d6) δ ppm 0.97 (s, 9H) 3.07-3.16 (m, 4H) 3.20 (t, J = 6.59 Hz, 2H) 3.41-3.51 (m, 2H) 3.54-3.65 (m, 4H) 4.47 (d, J = 9.52 Hz, 1H) 5.78 (s, 2H) 7.16 (t, J = 8.79 Hz, 2H) 7.24-7.37 (m, 3H) 7.41-7.50 (m, 1H) 7.61 (d, J = 10.25 Hz, 1H) 7.79 (d, J = 8.05 Hz, 1H) 8.16 (d, J = 8.79 Hz, 1H) 8.58 (t, J = 5.49 Hz, 1H) | 560 |
| 358 | 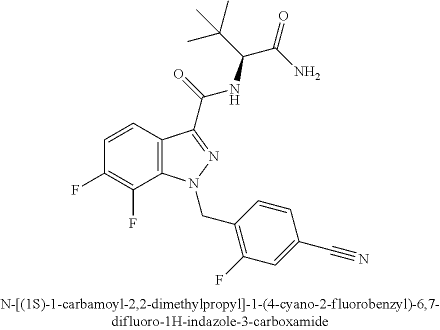 | ¹H NMR (400 MHz, DMSO-d6) δ ppm 7.96-8.02 (m, 1H), 7.90-7.95 (m, 1H), 7.73 (br. s., 1H), 7.62-7.67 (m, 1H), 7.56-7.61 (m, 1H), 7.34-7.43 (m, 1H), 7.30 (br. s., 1H), 7.21-7.27 (m, 1H), 5.96 (s, 2H), 4.43 (d, J = 9.52 Hz, 1H), 0.96 (s, 9H) | 444 |
| 359 | 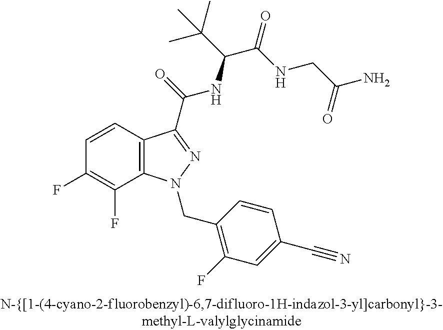 | ¹H NMR (400 MHz, DMSO-d6) δ ppm 8.49 (t, J = 5.67 Hz, 1H), 7.95-8.02 (m, 1H), 7.90-7.95 (m, 1H), 7.60-7.67 (m, 2H), 7.30-7.43 (m, 2H), 7.21-7.27 (m, 1H), 7.01 (br. s., 1H), 5.95 (s, 2H), 4.52 (d, J = 9.52 Hz, 1H), 3.61-3.75 (m, 2H), 0.97 (s, 9H) | 501 |
| 360 | 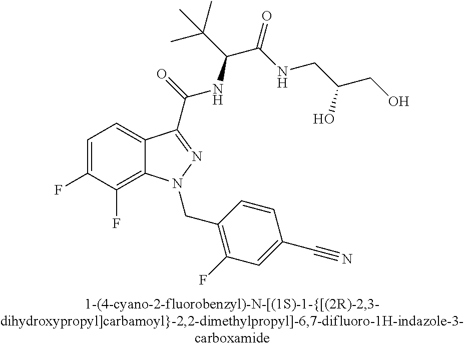 | ¹H NMR (400 MHz, DMSO-d6) δ ppm 8.28 (t, J = 5.67 Hz, 1H), 7.96-8.02 (m, 1H), 7.90-7.95 (m, 1H), 7.58-7.68 (m, 2H), 7.33-7.44 (m, 1H), 7.21-7.28 (m, 1H), 5.96 (s, 2H), 4.74 (d, J = 4.76 Hz, 1H), 4.49-4.57 (m, 2H), 3.43-3.52 (m, 1H), 3.25-3.31 (m, 2H), 3.13-3.22 (m, 1H), 3.00-3.10 (m, 1H), 0.95 (s, 9H) | 518 |
| 361 | 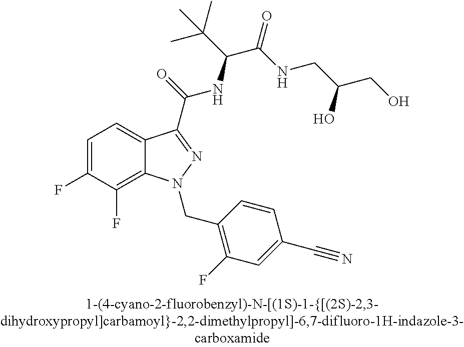 | ¹H NMR (400 MHz, DMSO-d6) δ ppm 8.27 (t, J = 5.49 Hz, 1H), 7.95-8.01 (m, 1H), 7.90-7.95 (m, 1H), 7.59-7.68 (m, 2H), 7.34-7.43 (m, 1H), 7.25 (t, J = 7.69 Hz, 1H), 5.96 (s, 2H), 4.72 (d, J = 5.12 Hz, 1H), 4.50-4.58 (m, 2H), 3.46-3.54 (m, 1H), 3.21-3.31 (m, 3H), 2.89-3.00 (m, 1H), 0.95 (s, 9H) | 518 |
| 362 | 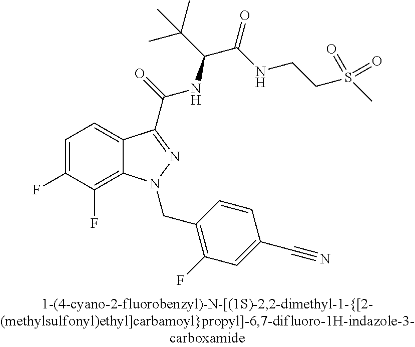 | ¹H NMR (400 MHz, DMSO-d6) δ ppm 8.60 (t, J = 5.49 Hz, 1H), 7.95-8.00 (m, 1H), 7.91-7.95 (m, 1H), 7.63-7.69 (m, 1H), 7.57-7.63 (m, 1H), 7.34-7.43 (m, 1H), 7.23-7.29 (m, 1H), 5.96 (s, 2H), 4.45 (d, J = 9.52 Hz, 1H), 3.40-3.57 (m, 2H), 3.19-3.30 (m, 2H), 2.99 (s, 3H), 0.95 (s, 9H) | 550 |
| 363 | 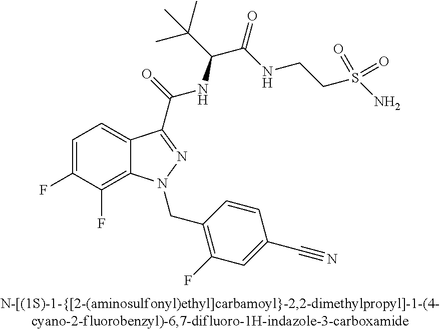 | ¹H NMR (400 MHz, DMSO-d6) δ ppm 8.52 (t, J = 5.49 Hz, 1H), 7.95-8.01 (m, 1H), 7.90-7.95 (m, 1H), 7.65 (d, J = 8.05 Hz, 1H), 7.60 (d, J = 9.52 Hz, 1H), 7.34-7.43 (m, 1H), 7.22-7.29 (m, 1H), 6.91 (s, 2H), 5.96 (s, 2H), 4.42 (d, J = 9.52 Hz, 1H), 3.36-3.56 (m, 2H), 3.04-3.19 (m, 2H), 0.95 (s, 9H) | 551 |
| 364 | 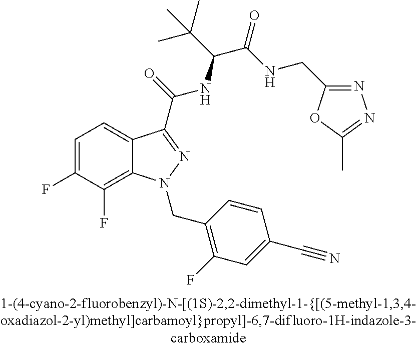 | ¹H NMR (400 MHz, DMSO-d6) δ ppm 9.06 (t, J = 5.67 Hz, 1H), 7.95-8.00 (m, 1H), 7.91-7.95 (m, 1H), 7.58-7.68 (m, 2H), 7.34-7.44 (m, 1H), 7.21-7.29 (m, 1H), 5.95 (s, 2H), 4.40-4.60 (m, 3H), 2.43 (s, 3H), 0.96 (s, 9H) | 540 |
| 365 | 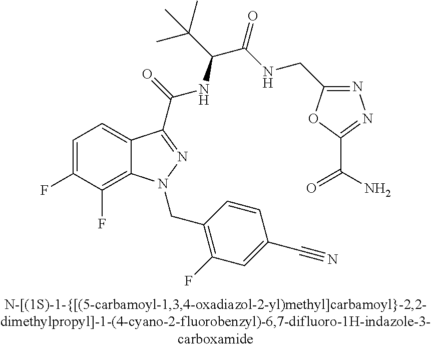 | ¹H NMR (400 MHz, DMSO-d6) δ ppm 9.16 (t, J = 5.49 Hz, 1H), 8.59 (s, 1H), 8.20 (s, 1H), 7.89-8.02 (m, 2H), 7.58-7.68 (m, 2H), 7.33-7.44 (m, 1H), 7.21-7.29 (m, 1H), 5.95 (s, 2H), 4.50-4.72 (m, 3H), 0.96 (s, 9H) | 569 |
| 366 | 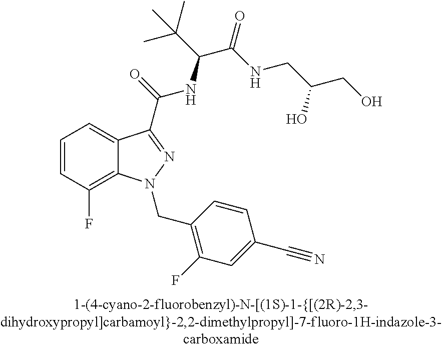 | ¹H NMR (400 MHz, DMSO-d6) δ ppm 8.25-8.31 (m, 1H), 7.97-8.03 (m, 1H), 7.89-7.94 (m, 1H), 7.59-7.66 (m, 2H), 7.23-7.35 (m, 2H), 7.12-7.19 (m, 1H), 5.97 (s, 2H), 4.50-4.57 (m, 1H), 3.43-3.53 (m, 1H), 3.24-3.31 (m, 2H), 3.12-3.22 (m, 1H), 3.00-3.11 (m, 1H), 0.95 (s, 9H) | 500 |
| 367 | 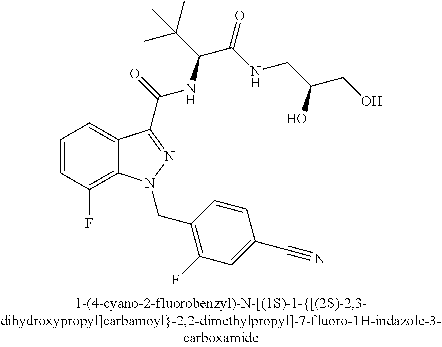 | ¹H NMR (400 MHz, DMSO-d6) δ ppm 8.24-8.30 (m, 1H), 7.98-8.03 (m, 1H), 7.89-7.95 (m, 1H), 7.59-7.66 (m, 2H), 7.23-7.36 (m, 2H), 7.11-7.18 (m, 1H), 5.97 (s, 2H), 4.72 (d, J = 4.76 Hz, 1H), 4.49-4.58 (m, 2H), 3.45-3.54 (m, 1H), 3.23-3.30 (m, 2H), 2.88-3.00 (m, 1H), 0.96 (s, 9H) | 500 |
| 368 | 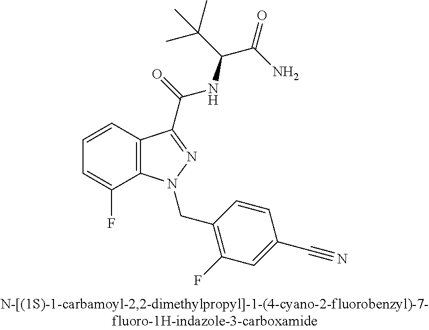 | ¹H NMR (400 MHz, DMSO-d6) δ ppm 7.98-8.04 (m, 1H), 7.88-7.95 (m, 1H), 7.69-7.77 (m, 1H), 7.57-7.65 (m, 2H), 7.23-7.36 (m, 3H), 7.10-7.18 (m, 1H), 5.97 (s, 2H), 4.45 (d, J = 9.52 Hz, 1H), 0.97 (s, 9H) | 426 |
| 369 | 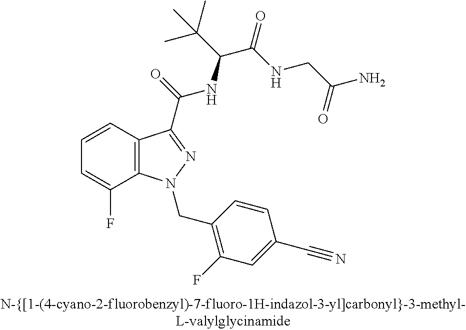 | ¹H NMR (400 MHz, DMSO-d6) δ ppm 8.49 (t, J = 5.67 Hz, 1H), 7.97-8.04 (m, 1H), 7.89-7.95 (m, 1H), 7.60-7.67 (m, 2H), 7.22-7.36 (m, 3H), 7.11-7.18 (m, 1H), 7.01 (br. s., 1H), 5.97 (s, 2H), 4.53 (d, J = 9.15 Hz, 1H), 3.61-3.74 (m, 2H), 0.97 (s, 9H) | 483 |
| 370 | 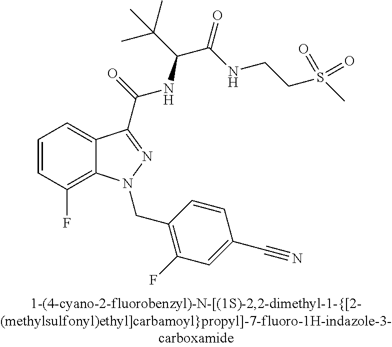 | ¹H NMR (400 MHz, DMSO-d6) δ ppm 8.56-8.65 (m, 1H), 7.96-8.03 (m, 1H), 7.90-7.96 (m, 1H), 7.57-7.68 (m, 2H), 7.23-7.37 (m, 2H), 7.12-7.20 (m, 1H), 5.97 (s, 2H), 4.46 (d, J = 9.52 Hz, 1H), 3.49 (td, J = 12.63, 6.22 Hz, 2H), 3.19-3.30 (m, 2H), 2.99 (s, 3H), 0.96 (s, 9H) | 532 |
| 371 | 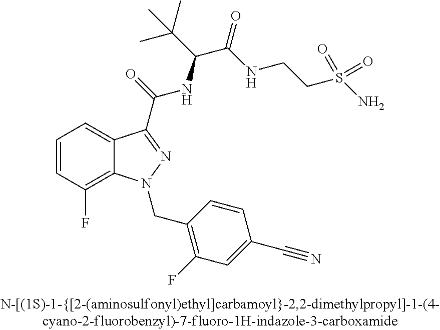 | ¹H NMR (400 MHz, DMSO-d6) δ ppm 8.48-8.56 (m, 1H), 7.97-8.03 (m, 1H), 7.89-7.96 (m, 1H), 7.57-7.67 (m, 2H), 7.23-7.36 (m, 2H), 7.13-7.20 (m, 1H), 6.92 (s, 2H), 5.97 (s, 2H), 4.43 (d, J = 9.52 Hz, 1H), 3.36-3.57 (m, 2H), 3.03-3.21 (m, 2H), 0.96 (s, 9H) | 533 |
| 372 | 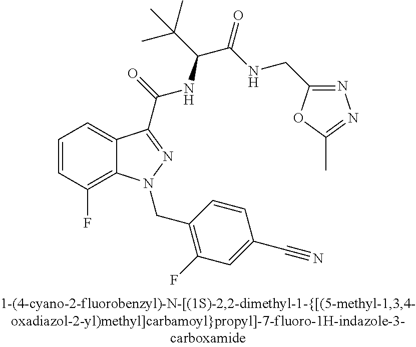 | ¹H NMR (400 MHz, DMSO-d6) δ ppm 9.06 (t, J = 5.67 Hz, 1H), 7.96-8.03 (m, 1H), 7.89-7.95 (m, 1H), 7.59-7.67 (m, 2H), 7.23-7.37 (m, 2H), 7.12-7.20 (m, 1H), 5.97 (s, 2H), 4.42-4.61 (m, 3H), 2.43 (s, 3H), 0.97 (s, 9H) | 522 |
| 373 | 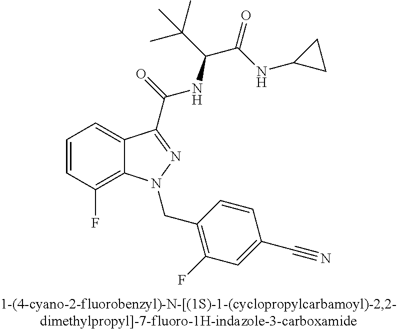 | ¹H NMR (400 MHz, DMSO-d6) δ ppm 8.33-8.38 (m, 1H), 7.96-8.03 (m, 1H), 7.89-7.96 (m, 1H), 7.61-7.66 (m, 1H), 7.54-7.60 (m, 1H), 7.24-7.36 (m, 2H), 7.12-7.19 (m, 1H), 5.98 (s, 2H), 4.39 (d, J = 9.88 Hz, 1H), 2.60-2.69 (m, 1H), 0.93 (s, 9H), 0.58-0.66 (m, 2H), 0.34-0.44 (m, 2H) | 466 |
| 374 | 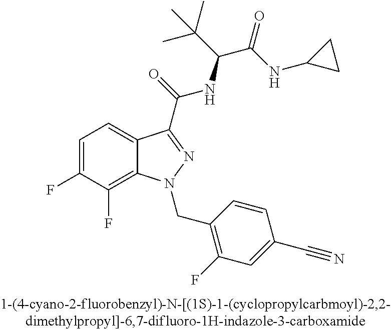 | ¹H NMR (400 MHz, DMSO-d6) δ ppm 8.33-8.39 (m, 1H), 7.96-8.00 (m, 1H), 7.90-7.96 (m, 1H), 7.63-7.68 (m, 1H), 7.54-7.59 (m, 1H), 7.34-7.43 (m, 1H), 7.22-7.29 (m, 1H), 5.96 (s, 2H), 4.38 (d, J = 9.88 Hz, 1H), 2.60-2.68 (m, 1H), 0.93 (s, 9H), 0.57-0.66 (m, 2H), 0.34-0.44 (m, 2H) | 484 |
| 375 | 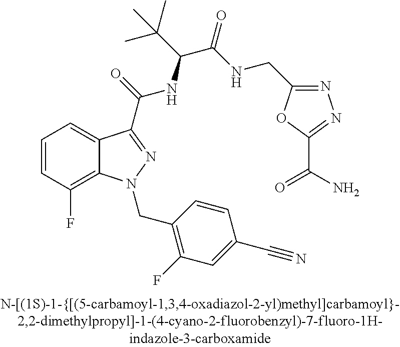 | ¹H NMR (400 MHz, DMSO-d6) δ ppm 9.11-9.20 (m, 1H), 8.59 (s, 1H), 8.20 (s, 1H), 7.97-8.03 (m, 1H), 7.89-7.95 (m, 1H), 7.58-7.67 (m, 2H), 7.23-7.37 (m, 2H), 7.11-7.20 (m, 1H), 5.97 (s, 2H), 4.51-4.71 (m, 3H), 0.97 (s, 9H) | 551 |
| 376 | 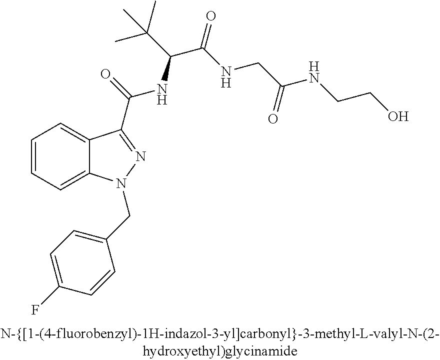 | ¹H NMR (400 MHz, DMSO-d6) δ ppm 0.98 (s, 9H) 3.11 (q, J = 5.37 Hz, 2H) 3.26-3.43 (m, 3H) 3.73 (d, J = 5.86 Hz, 1H) 4.54 (d, J = 9.52 Hz, 1H) 4.67 (br. s., 1H) 5.78 (s, 2H) 7.15 (t, J = 9.15 Hz, 2H) 7.24-7.36 (m, 3H) 7.41-7.50 (m, 1H) 7.62 (d, J = 9.52 Hz, 1H) 7.75-7.82 (m, 1H) 7.89 (t, J = 5.49 Hz, 1H) 8.17 (d, J = 8.79 Hz, 1H) 8.52 (t, J = 5.86 Hz, 1H) | 484 |
| 377 | 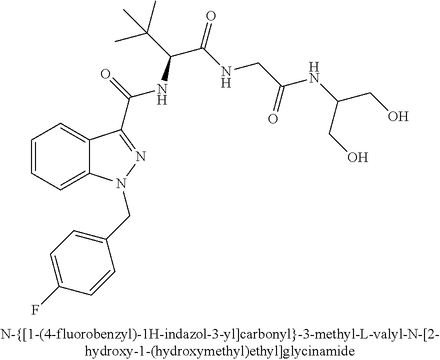 | ¹H NMR (400 MHz, DMSO-d6) δ ppm 0.92-1.07 (m, 9H) 3.38 (br. s., 4H) 3.63-3.86 (m, 3H) 4.55 (d, J = 9.52 Hz, 1H) 4.63 (br. s., 2H) 5.78 (s, 2H) 7.15 (t, J = 8.79 Hz, 2H) 7.24-7.35 (m, 3H) 7.45 (t, J = 7.32 Hz, 1H) 7.61 (t, J = 9.52 Hz, 2H) 7.78 (d, J = 8.79 Hz, 1H) 8.17 (d, J = 8.05 Hz, 1H) 8.52 (t, J = 5.49 Hz, 1H) | 514 |
| 378 | 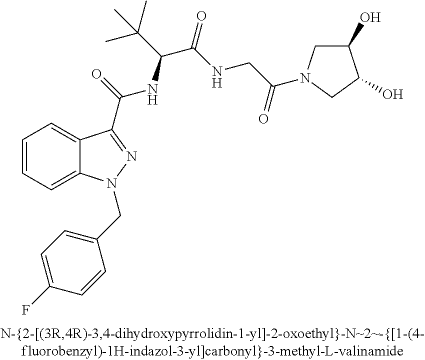 | ¹H NMR (400 MHz, DMSO-d6) δ ppm 1.00 (s, 9H) 3.23-3.32 (m, 2H) 3.36 (br. s., 2H) 3.58 (dd, J = 10.62, 4.03 Hz, 1H) 3.84-3.91 (m, 2H) 3.97 (br. s., 1H) 4.61 (d, J = 9.52 Hz, 1H) 5.16 (dd, J = 24.53, 3.29 Hz, 2H) 5.78 (s, 2H) 7.15 (t, J = 8.79 Hz, 2H) 7.24-7.35 (m, 3H) 7.45 (t, J = 7.69 Hz, 1H) 7.62 (d, J = 9.52 Hz, 1H) 7.78 (d, J = 8.79 Hz, 1H) 8.17 (d, J = 8.05 Hz, 1H) 8.48 (t, J = 5.49 Hz, 1H) | 526 |
| 379 | 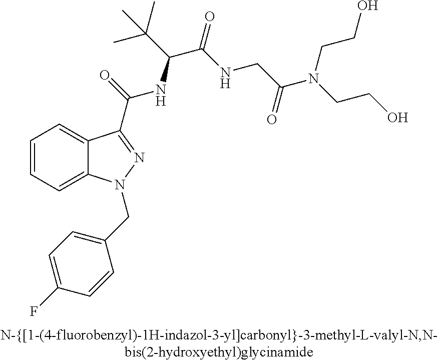 | ¹H NMR (400 MHz, DMSO-d6) δ ppm 0.99 (s, 9H) 3.28-3.33 (m, 2H) 3.35-3.40 (m, 2H) 3.41-3.57 (m, 4H) 4.07 (d, J = 3.66 Hz, 2H) 4.62 (d, J = 9.52 Hz, 1H) 4.67 (t, J = 5.13 Hz, 1H) 4.88 (t, J = 5.13 Hz, 1H) 5.78 (s, 2H) 7.15 (t, J = 8.79 Hz, 2H) 7.24-7.35 (m, 3H) 7.45 (t, J = 8.05 Hz, 1H) 7.63 (d, J = 9.52 Hz, 1H) 7.78 (d, J = 8.79 Hz, 1H) 8.17 (d, J = 8.05 Hz, 1H) 8.40 (t, J = 5.49 Hz, 1H) | 528 |
| 380 | 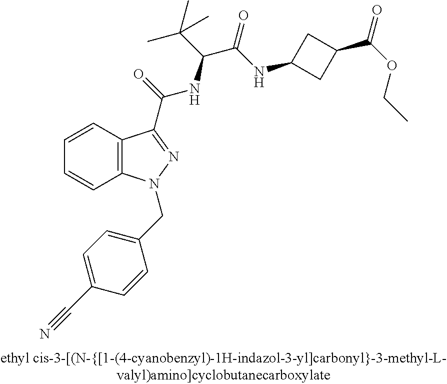 | ¹H NMR (400 MHz, DMSO-d6) δ ppm 0.84 (s, 1H) 0.94 (s, 9H) 1.15 (t, J = 7.14 Hz, 3H) 2.00-2.11 (m, 2H) 2.39-2.48 (m, 2H) 2.80 (t, J = 8.24 Hz, 1H) 4.04 (q, J = 7.20 Hz, 2H) 4.17 (d, J = 7.69 Hz, 1H) 4.42 (d, J = 9.88 Hz, 1H) 5.92 (s, 2H) 7.28-7.37 (m, 3H) 7.46 (t, J = 7.69 Hz, 1H) 7.57 (d, J = 9.88 Hz, 1H) 7.76 (d, J = 8.42 Hz, 1H) 7.79 (d, J = 8.05 Hz, 2H) 8.18 (d, J = 8.42 Hz, 1H) 8.62 (d, J = 8.05 Hz, 1H) | 516 |
| 381 | 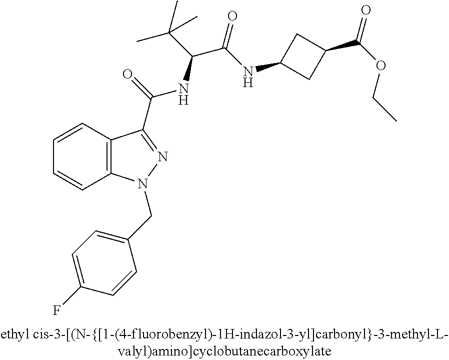 | ¹H NMR (400 MHz, DMSO-d6) δ ppm 0.84 (s, 1H) 0.95 (s, 9H) 1.16 (t, J = 7.14 Hz, 3H) 2.00-2.11 (m, 2H) 2.38 (br. s., 1H) 2.39-2.48 (m, 2H) 2.81 (t, J = 8.05 Hz, 1H) 4.04 (q, J = 7.08 Hz, 2H) 4.17 (d, J = 7.69 Hz, 1H) 4.42 (d, J = 9.88 Hz, 1H) 5.78 (s, 2H) 7.15 (t, J = 8.97 Hz, 2H) 7.26-7.34 (m, 3H) 7.45 (t, J = 7.69 Hz, 1H) 7.57 (d, J = 9.88 Hz, 1H) 7.79 (d, J = 8.42 Hz, 1H) 8.16 (d, J = 8.05 Hz, 1H) 8.61 (d, J = 7.69 Hz, 1H) | 509 |
| 382 | 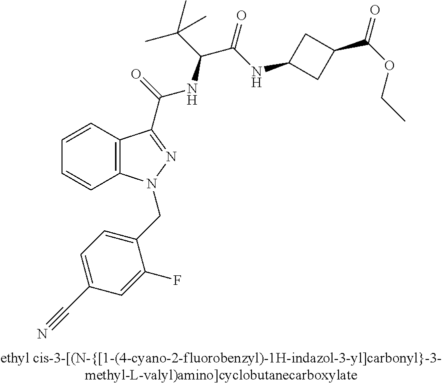 | ¹H NMR (400 MHz, DMSO-d6) δ ppm 0.84 (s, 1H) 0.93 (s, 9H) 1.15 (t, J = 7.14 Hz, 3H) 1.99-2.11 (m, 2H) 2.37 (br. s., 1H) 2.38-2.48 (m, 2H) 2.80 (t, J = 8.05 Hz, 1H) 4.04 (q, J = 6.95 Hz, 2H) 4.16 (d, J = 8.05 Hz, 1H) 4.40 (d, J = 9.88 Hz, 1H) 5.95 (s, 2H) 7.15 (t, J = 7.69 Hz, 1H) 7.31 (t, J = 7.50 Hz, 1H) 7.47-7.55 (m, 2H) 7.63 (d, J = 9.15 Hz, 1H) 7.78 (d, J = 8.42 Hz, 1H) 7.91 (d, J = 9.88 Hz, 1H) 8.18 (d, J = 8.42 Hz, 1H) 8.61 (d, J = 7.69 Hz, 1H) | 534 |
| 383 | 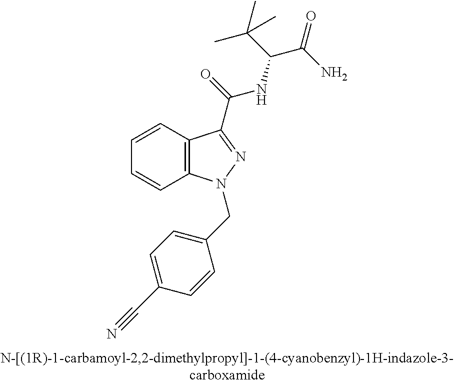 | ¹H NMR (400 MHz, DMSO-d6) δ ppm 0.97 (s, 9H) 4.46 (d, J = 9.88 Hz, 1H) 5.92 (s, 2H) 7.27-7.37 (m, 4H) 7.46 (t, J = 7.69 Hz, 1H) 7.60 (d, J = 9.88 Hz, 1H) 7.70-7.78 (m, 2H) 7.80 (d, J = 8.05 Hz, 2H) 8.19 (d, J = 8.05 Hz, 1H) | 390 |
| 384 | 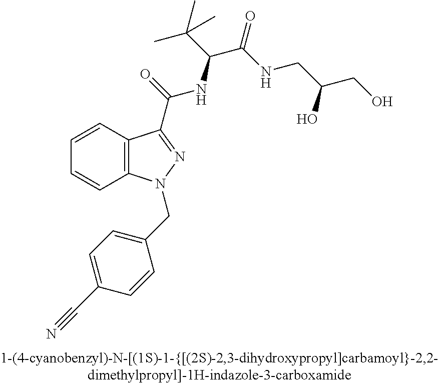 | ¹H NMR (400 MHz, DMSO-d6) δ ppm 0.96 (s, 9H) 2.90-3.00 (m, 1H) 3.24-3.31 (m, 3H) 3.50 (d, J = 6.22 Hz, 1H) 4.51-4.58 (m, 2H) 4.72 (d, J = 5.12 Hz, 1H) 5.92 (s, 2H) 7.28-7.37 (m, 3H) 7.46 (t, J = 7.50 Hz, 1H) 7.62 (d, J = 9.52 Hz, 1H) 7.76 (d, J = 8.42 Hz, 1H) 7.80 (d, J = 8.05 Hz, 2H) 8.19 (d, J = 8.05 Hz, 1H) 8.27 (t, J = 5.67 Hz, 1H) | 464 |
| 385 | 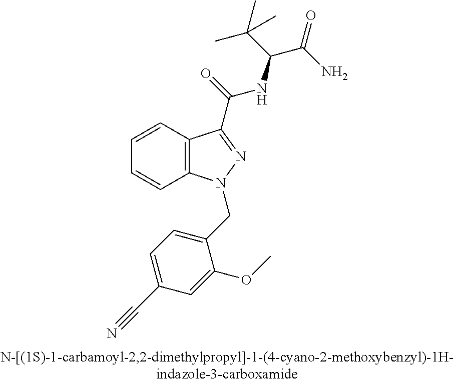 | ¹H NMR (400 MHz, DMSO-d6) δ ppm 0.96 (s, 9H) 3.89 (s, 3H) 4.44 (d, J = 9.52 Hz, 1H) 5.78 (d, J = 2.56 Hz, 2H) 6.79 (d, J = 7.69 Hz, 1H) 7.29 (dd, J = 12.26, 4.21 Hz, 2H) 7.32 (d, J = 5.12 Hz, 1H) 7.46 (t, J = 7.69 Hz, 1H) 7.51-7.59 (m, 2H) 7.67-7.76 (m, 2H) 8.18 (d, J = 8.05 Hz, 1H) | 420 |
| 386 | 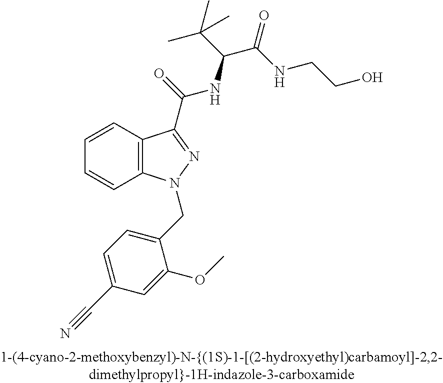 | ¹H NMR (400 MHz, DMSO-d6) δ ppm 0.94 (s, 9H) 3.11 (d, J = 5.86 Hz, 1H) 3.14-3.23 (m, 1H) 3.39 (q, J = 5.86 Hz, 2H) 3.89 (s, 3H) 4.48 (d, J = 9.52 Hz, 1H) 4.67 (t, J = 5.31 Hz, 1H) 5.78 (s, 2H) 6.80 (d, J = 7.69 Hz, 1H) 7.26-7.35 (m, 2H) 7.46 (t, J = 7.69 Hz, 1H) 7.51-7.59 (m, 2H) 7.71 (d, J = 8.42 Hz, 1H) 8.18 (d, J = 8.42 Hz, 1H) 8.30 (t, J = 5.49 Hz, 1H) | 464 |
| 387 | 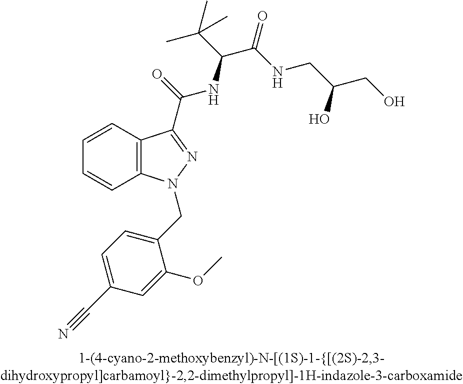 | ¹H NMR (400 MHz, DMSO-d6) δ ppm 0.95 (s, 9H) 2.95 (dd, J = 13.36, 6.04 Hz, 1H) 3.23-3.31 (m, 3H) 3.45-3.54 (m, 1H) 3.89 (s, 3H) 4.49-4.58 (m, 2H) 4.72 (d, J = 4.76 Hz, 1H) 5.78 (s, 2H) 6.80 (d, J = 7.69 Hz, 1H) 7.26-7.35 (m, 2H) 7.46 (t, J = 7.69 Hz, 1H) 7.54 (s, 1H) 7.58 (d, J = 9.52 Hz, 1H) 7.71 (d, J = 8.79 Hz, 1H) 8.18 (d, J = 8.42 Hz, 1H) 8.26 (t, J = 5.49 Hz, 1H) | 494 |
| 388 | 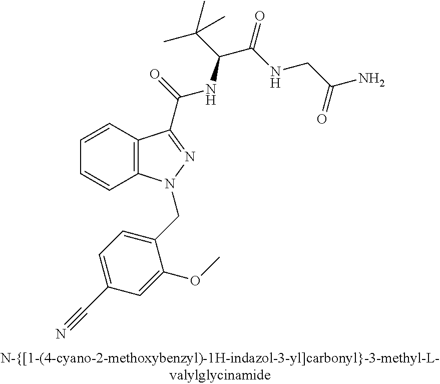 | ¹H NMR (400 MHz, DMSO-d6) δ ppm 0.97 (s, 9H) 3.67 (d, J = 5.86 Hz, 2H) 3.89 (s, 3H) 4.52 (d, J = 9.52 Hz, 1H) 5.78 (s, 2H) 6.80 (d, J = 8.05 Hz, 1H) 7.00 (br. s., 1H) 7.27-7.36 (m, 3H) 7.46 (t, J = 7.69 Hz, 1H) 7.54 (s, 1H) 7.58 (d, J = 9.52 Hz, 1H) 7.71 (d, J = 8.42 Hz, 1H) 8.18 (d, J = 8.42 Hz, 1H) 8.48 (t, J = 5.67 Hz, 1H) | 477 |
| 389 | 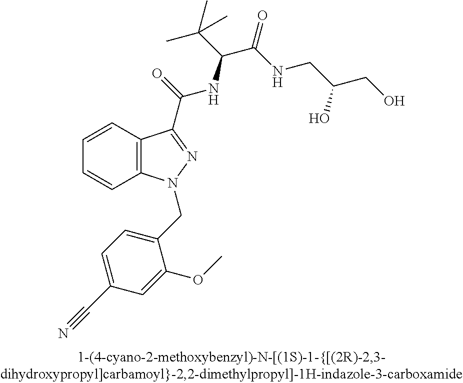 | ¹H NMR (400 MHz, DMSO-d6) δ ppm 0.95 (s, 9H) 3.01-3.10 (m, 1H) 3.12-3.22 (m, 1H) 3.28 (t, J = 5.67 Hz, 2H) 3.43-3.52 (m, 1H) 3.89 (s, 3H) 4.49-4.57 (m, 2H) 4.74 (d, J = 5.12 Hz, 1H) 5.78 (s, 2H) 6.80 (d, J = 7.69 Hz, 1H) 7.26-7.35 (m, 2H) 7.46 (t, J = 7.50 Hz, 1H) 7.54 (s, 1H) 7.57 (d, J = 9.88 Hz, 1H) 7.71 (d, J = 8.79 Hz, 1H) 8.18 (d, J = 8.05 Hz, 1H) 8.27 (t, J = 5.49 Hz, 1H) | 494 |
| 390 | 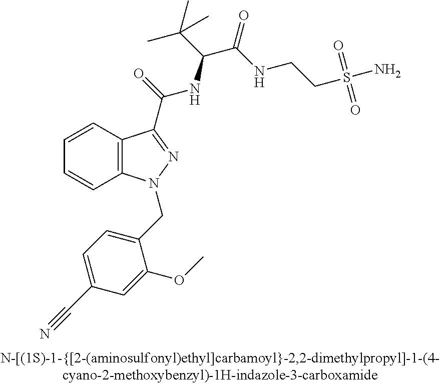 | ¹H NMR (400 MHz, DMSO-d6) δ ppm 0.95 (s, 9H) 3.06-3.17 (m, 2H) 3.36-3.45 (m, 1H) 3.49 (dd, J = 8.42, 5.49 Hz, 1H) 3.89 (s, 3H) 4.42 (d, J = 9.88 Hz, 1H) 5.78 (s, 2H) 6.82 (d, J = 8.05 Hz, 1H) 6.91 (s, 2H) 7.27-7.36 (m, 2H) 7.46 (t, J = 7.50 Hz, 1H) 7.51-7.58 (m, 2H) 7.72 (d, J = 8.42 Hz, 1H) 8.17 (d, J = 8.05 Hz, 1H) 8.51 (t, J = 5.49 Hz, 1H) | 527 |
| 391 | 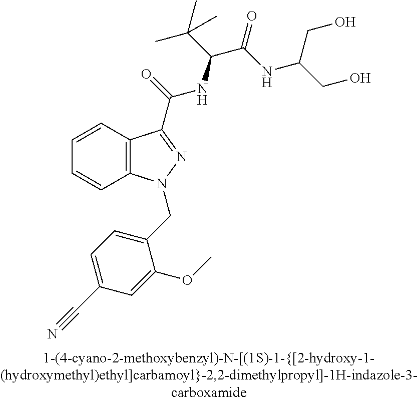 | ¹H NMR (400 MHz, DMSO-d6) δ ppm 0.75 (s, 3H) 0.84 (s, 4H) 1.28 (s, 4H) 1.33 (s, 3H) 3.44-3.49 (m, 1H) 3.52-3.57 (m, 1H) 3.81-3.91 (m, 3H) 4.18-4.30 (m, 1H) 4.43-4.52 (m, 1H) 4.93-5.01 (m, 1H) 5.77 (br. s., 2H) 6.82 (d, J = 7.69 Hz, 1H) 7.29-7.38 (m, 2H) 7.45-7.55 (m, 2H) 7.78 (d, J = 8.05 Hz, 1H) 7.99-8.11 (m, 1H) | 494 |
| 392 | 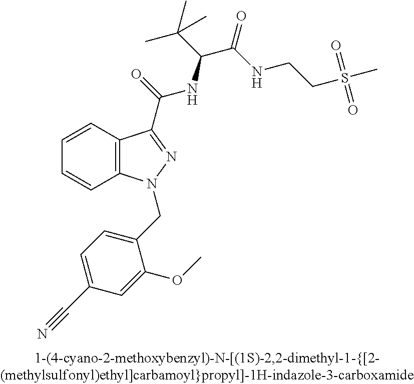 | ¹H NMR (400 MHz, DMSO-d6) δ ppm 0.95 (s, 9H) 2.99 (s, 3H) 3.26 (q, J = 7.20 Hz, 2H) 3.42-3.54 (m, 2H) 3.89 (s, 3H) 4.45 (d, J = 9.52 Hz, 1H) 5.78 (s, 2H) 6.81 (d, J = 7.69 Hz, 1H) 7.27-7.36 (m, 2H) 7.46 (t, J = 7.69 Hz, 1H) 7.51-7.59 (m, 2H) 7.72 (d, J = 8.42 Hz, 1H) 8.17 (d, J = 8.05 Hz, 1H) 8.60 (t, J = 5.49 Hz, 1H) | 526 |
| 393 | 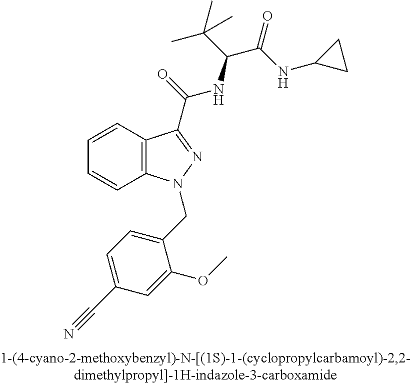 | ¹H NMR (400 MHz, DMSO-d6) δ ppm 0.34-0.43 (m, 1H) 0.55-0.64 (m, 1H) 0.86 (s, 1H) 0.89-0.99 (m, 8H) 1.18-1.29 (m, 3H) 1.67-1.76 (m, 1H) 2.64 (dt, J = 7.50, 3.57 Hz, 1H) 2.69-2.75 (m, 2H) 2.95-3.05 (m, 2H) 3.89 (s, 2H) 4.38 (d, J = 9.52 Hz, 1H) 5.78 (d, J = 2.20 Hz, 1H) 6.81 (d, J = 7.69 Hz, 1H) 7.27-7.36 (m, 1H) 7.38-7.48 (m, 1H) 7.50-7.56 (m, 1H) 7.71 (dd, J = 8.42, 4.76 Hz, 1H) 8.17 (d, J = 8.05 Hz, 1H) 8.36 (d, J = 4.03 Hz, 1H) | 460 |
| 394 | 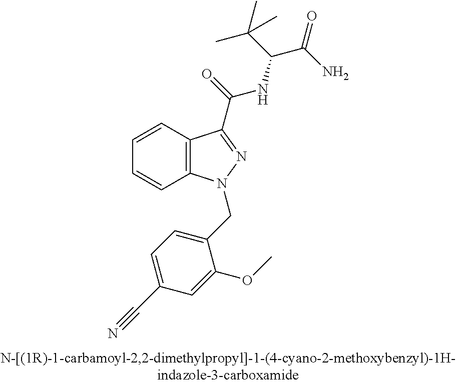 | ¹H NMR (400 MHz, DMSO-d6) δ ppm 0.96 (s, 9H) 3.89 (s, 3H) 4.44 (d, J = 9.52 Hz, 1H) 5.78 (d, J = 2.56 Hz, 2H) 6.79 (d, J = 8.05 Hz, 1H) 7.29 (dd, J = 12.08, 4.03 Hz, 2H) 7.32 (d, J = 5.12 Hz, 1H) 7.46 (t, J = 7.50 Hz, 1H) 7.51-7.59 (m, 2H) 7.67-7.76 (m, 2H) 8.18 (d, J = 8.42 Hz, 1H) | 420 |
| 395 | 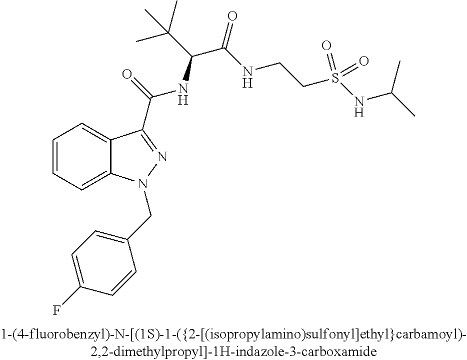 | ¹H NMR (400 MHz, DMSO-d6) δ ppm 0.96 (s, 9H) 1.08 (d, J = 6.59 Hz, 7H) 3.06-3.16 (m, 2H) 4.44 (d, J = 9.52 Hz, 2H) 5.78 (s, 2H) 7.07-7.21 (m, 3H) 7.24-7.37 (m, 3H) 7.42-7.50 (m, 2H) 7.60 (d, J = 9.52 Hz, 1H) 7.79 (d, J = 8.79 Hz, 1H) 8.15 (d, J = 8.05 Hz, 1H) 8.46-8.56 (m, 1H) | 532 |
| 396 | 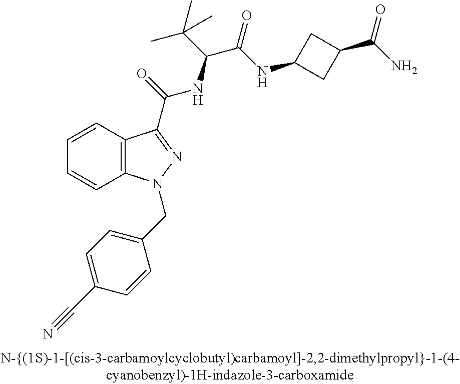 | ¹H NMR (400 MHz, DMSO-d6) δ ppm 0.94 (s, 9H) 2.00 (t, J = 10.25 Hz, 2H) 2.22-2.33 (m, 2H) 2.60 (t, J = 7.87 Hz, 1H) 4.10 (d, J = 8.05 Hz, 1H) 4.43 (d, J = 9.52 Hz, 1H) 5.92 (s, 2H) 6.75 (br. s., 1H) 7.22 (br. s., 1H) 7.27-7.37 (m, 3H) 7.46 (t, J = 7.69 Hz, 1H) 7.58 (d, J = 9.52 Hz, 1H) 7.76 (d, J = 8.42 Hz, 1H) 7.80 (d, J = 8.42 Hz, 2H) 8.18 (d, J = 8.42 Hz, 1H) 8.57 (d, J = 7.69 Hz, 1H) | 487 |
| 397 | 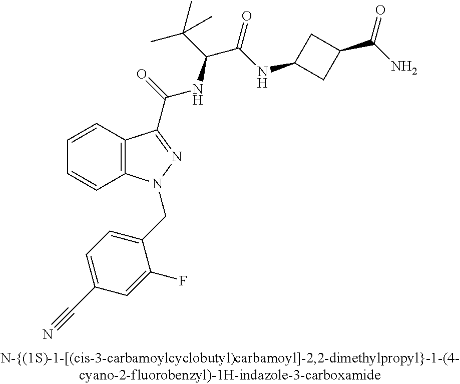 | ¹H NMR (400 MHz, DMSO-d6) δ ppm 0.93 (s, 9H) 1.96-2.07 (m, 2H) 2.21-2.32 (m, 2H) 2.60 (t, J = 8.05 Hz, 1H) 3.58 (s, 1H) 4.09 (d, J = 8.05 Hz, 1H) 4.42 (d, J = 9.52 Hz, 1H) 5.87 (d, J = 7.32 Hz, 1H) 5.95 (s, 1H) 6.75 (br. s., 1H) 7.12-7.19 (m, 1H) 7.22 (br. s., 1H) 7.25-7.34 (m, 1H) 7.45-7.56 (m, 2H) 7.63 (d, J = 8.05 Hz, 1H) 7.78 (d, J = 8.42 Hz, 1H) 7.91 (d, J = 9.88 Hz, 1H) 8.18 (d, J = 8.05 Hz, 1H) 8.56 (d, J = 7.69 Hz, 1H) | 505 |
| 398 | 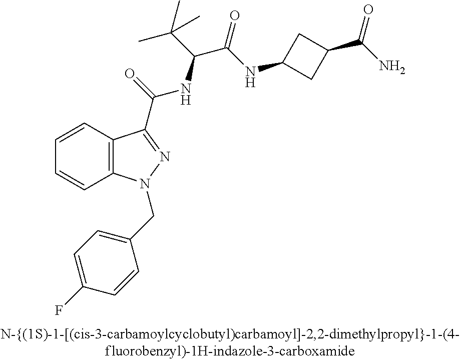 | ¹H NMR (400 MHz, DMSO-d6) δ ppm 0.95 (s, 9H) 2.01 (t, J = 10.07 Hz, 2H) 2.22-2.33 (m, 2H) 2.61 (t, J = 7.87 Hz, 1H) 4.10 (d, J = 8.05 Hz, 1H) 4.43 (d, J = 9.52 Hz, 1H) 5.77 (s, 2H) 6.75 (br. s., 1H) 7.11-7.20 (m, 2H) 7.22 (br. s., 1H) 7.24-7.34 (m, 3H) 7.45 (t, J = 7.69 Hz, 1H) 7.59 (d, J = 9.52 Hz, 1H) 7.78 (d, J = 8.42 Hz, 1H) 8.16 (d, J = 8.05 Hz, 1H) 8.57 (d, J = 7.69 Hz, 1H) | 480 |
| 399 | 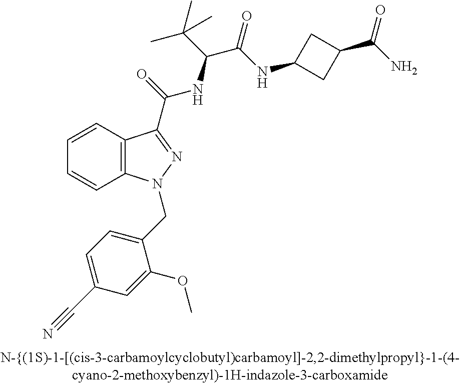 | ¹H NMR (400 MHz, DMSO-d6) δ ppm 0.93 (s, 9H) 2.00 (t, J = 9.88 Hz, 2H) 2.24-2.33 (m, 2H) 2.60 (t, J = 7.87 Hz, 1H) 3.88 (s, 3H) 4.09 (d, J = 7.69 Hz, 1H) 4.42 (d, J = 9.88 Hz, 1H) 5.78 (br. s., 2H) 6.74 (br. s., 1H) 6.80 (d, J = 7.69 Hz, 1H) 7.22 (br. s., 1H) 7.25-7.34 (m, 2H) 7.46 (t, J = 7.69 Hz, 1H) 7.50-7.58 (m, 2H) 7.71 (d, J = 8.42 Hz, 1H) 8.17 (d, J = 8.05 Hz, 1H) 8.56 (d, J = 7.69 Hz, 1H) | 517 |
| 400 | 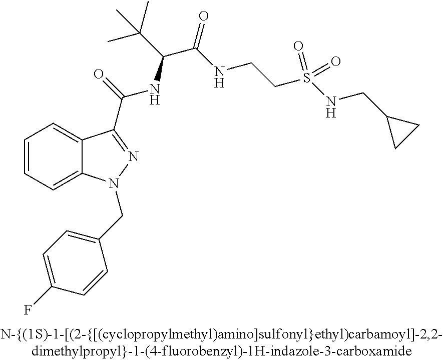 | ¹H NMR (400 MHz, DMSO-d6) δ ppm 0.13 (d, J = 3.66 Hz, 2H) 0.38 (d, J = 6.59 Hz, 2H) 0.84-0.93 (m, 1H) 0.96 (s, 9H) 2.78 (t, J = 6.59 Hz, 2H) 3.07-3.22 (m, 2H) 3.38-3.50 (m, 2H) 4.44 (d, J = 9.52 Hz, 1H) 5.78 (s, 2H) 7.16 (t, J = 9.15 Hz, 2H) 7.21-7.27 (m, 1H) 7.28-7.35 (m, 3H) 7.45 (t, J = 7.69 Hz, 1H) 7.60 (d, J = 9.52 Hz, 1H) 7.79 (d, J = 8.79 Hz, 1H) 8.15 (d, J = 8.79 Hz, 1H) 8.51 (t, J = 5.49 Hz, 1H) | 544 |
| 401 | 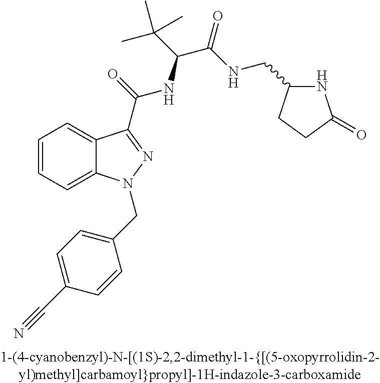 | ¹H NMR (400 MHz, DMSO-d6) δ ppm 8.36 (ddd, J = 15.56, 5.86, 5.67 Hz, 1H) 8.18 (d, J = 8.42 Hz, 1H) 7.73-7.83 (m, 3H) 7.56-7.65 (m, 2H) 7.47 (t, J = 7.50 Hz, 1H) 7.27-7.38 (m, 3H) 5.92 (s, 2H) 4.50 (dd, J = 9.52, 5.86 Hz, 1H) 3.53-3.59 (m, 1H) 3.25 (ddd, J = 13.27, 5.40, 5.12 Hz, 1H) 3.15 (t, J = 5.67 Hz, 1H) 2.95-3.04 (m, 1H) 1.97-2.18 (m, 3H) 1.64-1.76 (m, 1H) 0.96 (s, 9H). | 487 |
| 402 | 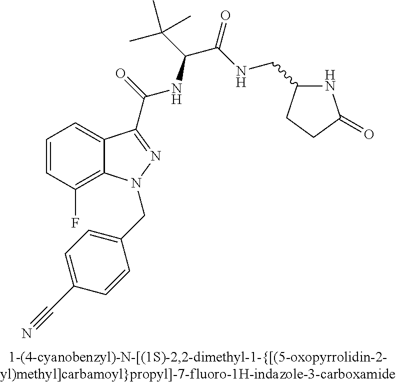 | ¹H NMR (400 MHz, DMSO-d6) δ ppm 8.38 (dt, J = 16.11, 5.67 Hz, 1H) 8.01 (d, J = 7.69 Hz, 1H) 7.80 (d, J = 8.42 Hz, 2H) 7.55-7.69 (m, 2H) 7.22-7.34 (m, 4H) 5.93 (s, 2H) 4.50 (dd, J = 9.52, 6.22 Hz, 1H) 3.54-3.60 (m, 1H) 3.21-3.28 (m, 1H) 3.15 (t, J = 5.86 Hz, 1H) 2.96-3.05 (m, 1H) 1.98-2.19 (m, 3H) 1.64-1.76 (m, 1H) 0.97 (s, 9H). | 505 |
| 403 | 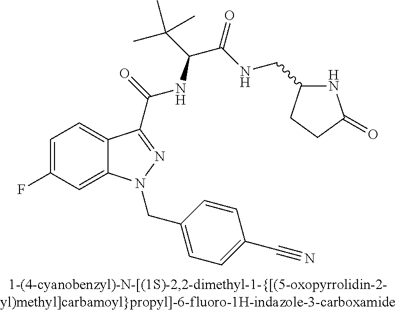 | ¹H NMR (400 MHz, DMSO-d6) δ ppm 8.36 (dt, J = 16.01, 5.72 Hz, 1H) 8.18 (dd, J = 8.79, 5.49 Hz, 1H) 7.72-7.84 (m, 3H) 7.58 (d, J = 8.05 Hz, 2H) 7.37 (d, J = 7.69 Hz, 2H) 7.20 (td, J = 9.15, 2.20 Hz, 1H) 5.87 (s, 2H) 4.48 (dd, J = 9.70, 6.41 Hz, 1H) 3.55 (d, J = 5.86 Hz, 1H) 3.20-3.28 (m, 1H) 3.14 (t, J = 5.67 Hz, 1H) 2.95-3.03 (m, 1H) 1.95-2.18 (m, 3H) 1.61-1.74 (m, 1H) 0.95 (s, 9H). | 505 |
| 404 | 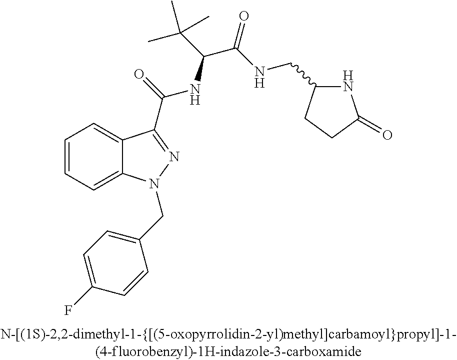 | ¹H NMR (400 MHz, DMSO-d6) δ ppm 8.37 (ddd, J = 15.10, 5.77, 5.49 Hz, 1H) 8.16 (d, J = 8.05 Hz, 1H) 7.79 (d, J = 8.42 Hz, 1H) 7.56-7.63 (m, 2H) 7.45 (t, J = 7.50 Hz, 1H) 7.26-7.34 (m, 3H) 7.15 (t, J = 8.97 Hz, 2H) 5.78 (s, 2H) 4.50 (dd, J = 9.70, 6.04 Hz, 1H) 3.56 (br. s., 1H) 3.22-3.29 (m, 1H) 3.15 (t, J = 5.67 Hz, 1H) 2.95-3.04 (m, 1H) 1.97-2.19 (m, 3H) 1.64-1.75 (m, 1H) 0.97 (s, 9H). | 480 |
| 405 | 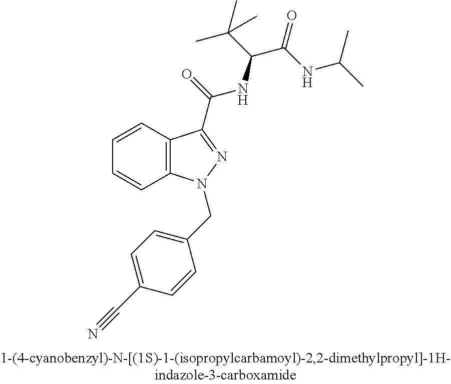 | ¹H NMR (400 MHz, DMSO-d6) δ ppm 0.95 (s, 9H) 1.05 (dd, J = 6.59, 2.93 Hz, 6H) 3.80-3.91 (m, 1H) 4.45 (d, J = 9.88 Hz, 1H) 5.92 (s, 2H) 7.28-7.37 (m, 3H) 7.47 (t, J = 7.50 Hz, 1H) 7.58 (d, J = 9.88 Hz, 1H) 7.76 (d, J = 8.42 Hz, 1H) 7.80 (d, J = 8.42 Hz, 2H) 8.13-8.22 (m, 2H) | 432 |
| 406 | 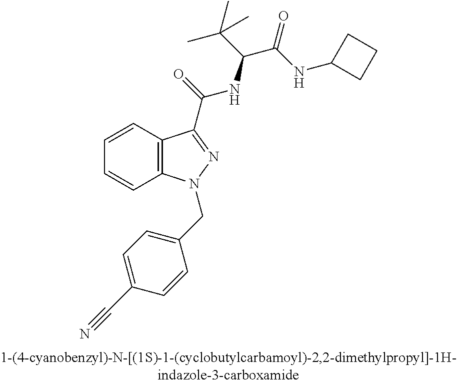 | ¹H NMR (400 MHz, DMSO-d6) δ ppm 0.95 (s, 9H) 1.05 (dd, J = 6.59, 2.93 Hz, 6H) 3.80-3.91 (m, 1H) 4.45 (d, J = 9.88 Hz, 1H) 5.92 (s, 2H) 7.28-7.37 (m, 3H) 7.47 (t, J = 7.50 Hz, 1H) 7.58 (d, J = 9.88 Hz, 1H) 7.76 (d, J = 8.42 Hz, 1H) 7.80 (d, J = 8.42 Hz, 2H) 8.13-8.22 (m, 2H) | 444 |
| 407 | 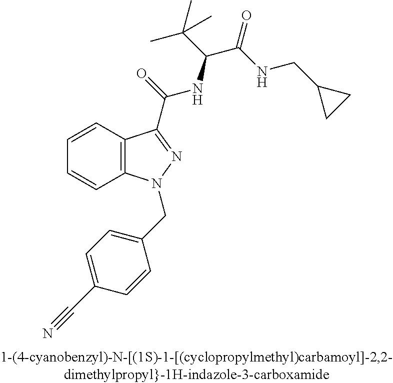 | ¹H NMR (400 MHz, DMSO-d6) δ ppm 0.14 (q, J = 4.64 Hz, 2H) 0.34-0.43 (m, 2H) 0.85-0.94 (m, 1H) 0.97 (s, 9H) 2.84-2.92 (m, 1H) 2.98-3.07 (m, 1H) 4.49 (d, J = 9.88 Hz, 1H) 5.92 (s, 2H) 7.28-7.37 (m, 3H) 7.46 (t, J = 7.50 Hz, 1H) 7.60 (d, J = 9.52 Hz, 1H) 7.76 (d, J = 8.42 Hz, 1H) 7.80 (d, J = 8.05 Hz, 2H) 8.18 (d, J = 8.05 Hz, 1H) 8.39 (t, J = 5.49 Hz, 1H) | 444 |
| 408 | 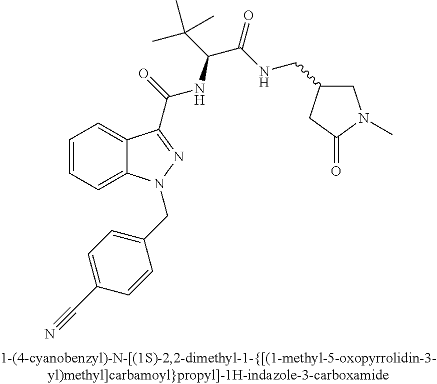 | ¹H NMR (400 MHz, DMSO-d6) δ ppm 8.46 (br. s., 1H) 8.18 (d, J = 8.05 Hz, 1H) 7.74-7.83 (m, 3H) 7.60 (d, J = 9.52 Hz, 1H) 7.47 (t, J = 7.50 Hz, 1H) 7.26-7.39 (m, 3H) 5.92 (s, 2H) 4.47 (dd, J = 9.70, 2.01 Hz, 1H) 3.39 (d, J = 4.76 Hz, 1H) 3.23 (dt, J = 13.09, 6.45 Hz, 1H) 2.96-3.06 (m, 2H) 2.65 (d, J = 2.56 Hz, 3H) 2.38-2.47 (m, 1H) 2.31 (dt, J = 16.47, 8.24 Hz, 1H) 1.99 (ddd, J = 16.66, 6.77, 6.59 Hz, 1H) 0.96 (s, 9H). | 501 |
| 409 | 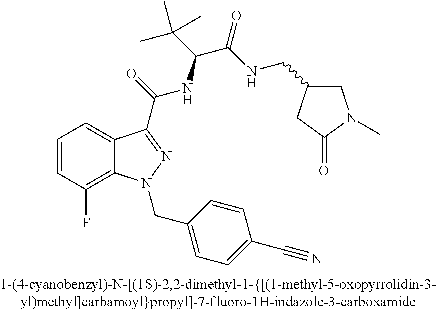 | ¹H NMR (400 MHz, DMSO-d6) δ ppm 8.47 (br. s., 1H) 8.00 (d, J = 7.32 Hz, 1H) 7.80 (d, J = 8.42 Hz, 2H) 7.64 (d, J = 9.52 Hz, 1H) 7.23-7.35 (m, 4H) 5.93 (s, 2H) 4.48 (dd, J = 9.70, 1.65 Hz, 1H) 3.37-3.42 (m, 1H) 3.24 (ddd, J = 12.99, 6.41, 6.22 Hz, 1H) 2.97-3.08 (m, 2H) 2.65 (d, J = 1.83 Hz, 3H) 2.39-2.48 (m, 1H) 2.31 (dt, J = 16.47, 8.24 Hz, 1H) 1.99 (dt, J = 16.47, 6.59 Hz, 1H) 0.96 (s, 9H). | 519 |
| 410 | 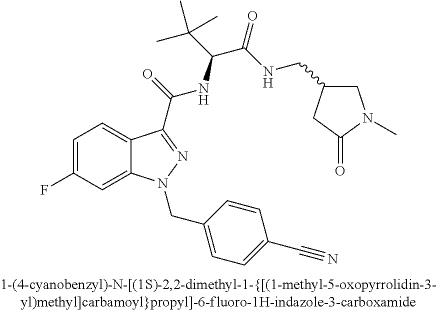 | ¹H NMR (400 MHz, DMSO-d6) δ ppm 8.45 (br. s., 1H) 8.18 (dd, J = 8.97, 5.31 Hz, 1H) 7.71-7.84 (m, 3H) 7.58 (d, J = 9.88 Hz, 1H) 7.38 (d, J = 8.42 Hz, 2H) 7.20 (td, J = 9.15, 2.20 Hz, 1H) 5.87 (s, 2H) 4.41-4.50 (m, 1H) 3.39 (d, J = 4.76 Hz, 1H) 3.23 (ddd, J = 13.27, 6.50, 6.22 Hz, 1H) 2.96-3.08 (m, 2H) 2.65 (d, J = 2.56 Hz, 3H) 2.38-2.47 (m, 1H) 2.30 (ddd, J = 16.66, 8.24, 8.05 Hz, 1H) 1.99 (ddd, J = 16.66, 6.59, 6.41 Hz, 1H) 0.95 (s, 9H). | 519 |
| 411 | 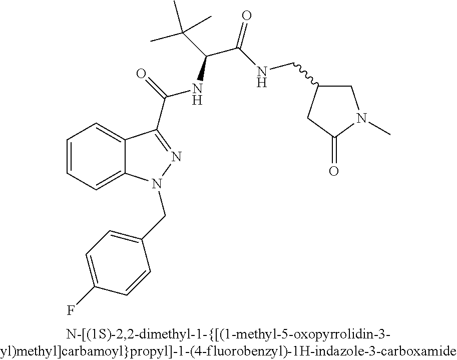 | ¹H NMR (400 MHz, DMSO-d6) δ ppm 8.46 (t, J = 4.76 Hz, 1H) 8.16 (d, J = 8.05 Hz, 1H) 7.79 (d, J = 8.79 Hz, 1H) 7.60 (d, J = 9.52 Hz, 1H) 7.45 (t, J = 7.69 Hz, 1H) 7.25-7.34 (m, 3H) 7.15 (t, J = 8.79 Hz, 2H) 5.77 (s, 2H) 4.47 (dd, J = 9.70, 2.01 Hz, 1H) 3.38-3.44 (m, 1H) 3.24 (ddd, J = 13.27, 6.50, 6.22 Hz, 1H) 2.96-3.08 (m, 2H) 2.65 (d, J = 2.56 Hz, 3H) 2.39-2.47 (m, 1H) 2.31 (dt, J = 16.56, 8.37 Hz, 1H) 2.00 (ddd, J = 16.66, 6.77, 6.59 Hz, 1H) 0.96 (s, 9H). | 494 |
| 412 | 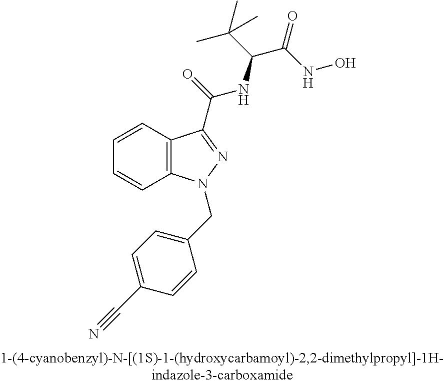 | ¹H NMR (400 MHz, DMSO-d6) δ ppm 0.89 (s, 1H) 0.91-1.00 (m, 9H) 4.33 (d, J = 9.88 Hz, 1H) 5.92 (d, J = 4.39 Hz, 2H) 7.27-7.37 (m, 3H) 7.47 (t, J = 7.50 Hz, 1H) 7.56 (d, J = 9.88 Hz, 1H) 7.71-7.82 (m, 3H) 8.18 (d, J = 8.05 Hz, 1H) 9.04 (s, 1H) 10.90 (s, 1H) | 406 |
| 413 | 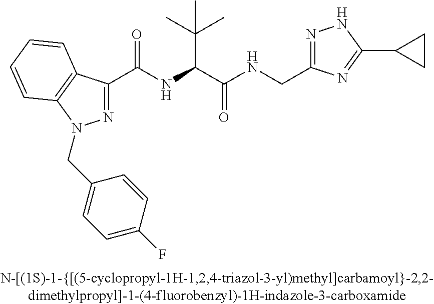 | ¹H NMR (400 MHz, CDCl3-d6) δ ppm δ 0.828 (s, 4H) 0.986 (s, 9H) 4.322 (s, 2) 4.482 (s, 2H) 5.614 (s, 2H) 6.925-6.968 (t, J = 17.2 Hz, 2H) 7.191-7.227 (m, 4H) 7.334-7.352 (t, J = 7.2 Hz, 1H) 7.486-7.507 (d, J = 8.4 Hz, 1H) 7.797 (s, 1H) 8.116-8.136 (d, J = 8 Hz, 1H) | 504 |
| 414 | 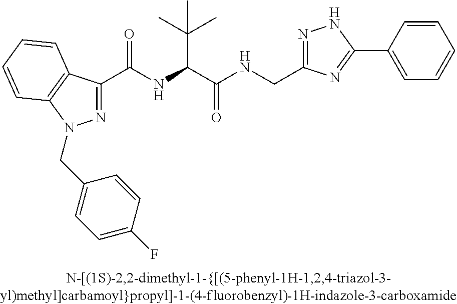 | ¹H NMR (400 MHz, CDCl3-d6) δ ppm 1.097 (s, 9H) 4.437-4.458 (d, J = 8.4 Hz, 1H) 4.689 (m, 2H) 5.504 (s, 2H) 2.312-2.347 (t, J = 14 Hz, 2H) 4.389-4.488 (m, 4H) 4.534-4.547 (m, 1H) 7.181-7.249 (m, 1H) 7.345-7.379 (m, 2H) 7.487 (s, 1H) 7.761-7.693 (d, J = 12.8 Hz, 1H) 8.240-8.261 (d, J = 8.4 Hz, 1H) | 540 |
| 415 | 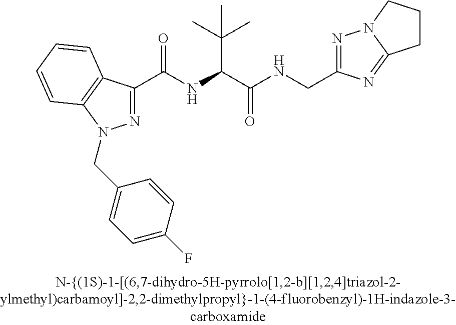 | ¹H NMR (400 MHz, CDCl3-d6) δ ppm 1.129 (s, 9H) 2.598-2.654 (q, 2H) 2.800-2.837 (t, J = 14.8 Hz, 2H) 3.990-4.026 (t, J = 14.4 Hz, 2H) 4.465-4.495 (q, 3H) 5.512 (s, 2H) 6.554-6.577 (t, J = 9.2 Hz, 1H) 6.909-6.952 (t, J = 17.2 Hz, 2H) 7.122-7.156 (q, 3H) 7.180-7.303 (m, 2H) 7.682-7.706 (d, J = 9.6 Hz, 1H) 8.258-8.278 (d, J = 8 Hz, 1H) | 504 |
| 416 | 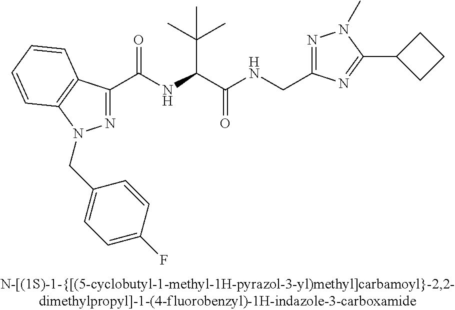 | ¹H NMR (400 MHz, CDCl3-d6) δ ppm 1.119 (s, 9H) 1.878-2.085 (m, 4H) 2.317-2.353 (m, 2H), 3.386-3.406 (m, 1H) 3.640 (s, 3H) 4.401-4.490 (m, 3H) 5.587 (s, 1H) 5.998 (s, 1H) 6.980-7.023 (m, 2H) 7.192-7.257 (m, 3H) 7.261-7.356 (m, 3H) 8.335-8.353 (d, J = 7.2 Hz, 1H) | 531 |
| 417 | 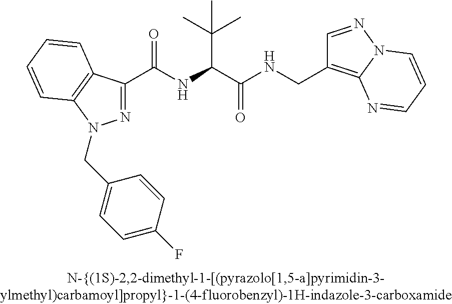 | ¹H NMR (400 MHz, CDCl3-d6) δ ppm δ 0.951 (s, 9H) 4.412-4.504 (m, 2H) 4.562-4.599 (d, J = 14.8 Hz 1H) 5.614 (s, 2H) 6.854-6.872 (m, 1H) 6.930-6.974 (t, 2H) 7.175-7.231 (m. 3H) 7.329-7.504 (m, 2H) 7.799 (s, 1H) 8.375-8.389 (m, 2H) 8.728-8.749 (q, 1H) | 514 |
| 418 | 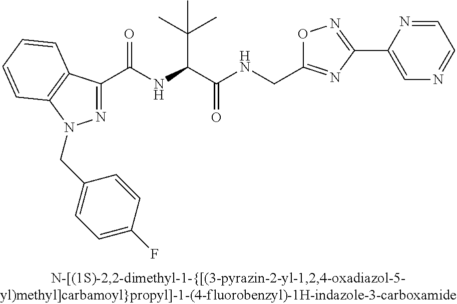 | ¹H NMR (400 MHz, CDCl3-d6) δ ppm δ 1.129 (s, 9H) 4.582-4.687 (m, 2H) 4.835-4.849 (d, J = 5.6 Hz, 1H) 5.516 (s, 2H) 7.002-7.023 (m, 2H) 7.183-7.252 (m, 4H) 7.254-7.265 (m, 2H) 8.228 (s, 1H) 8.726 (s, 1H) | 543 |
| 419 | 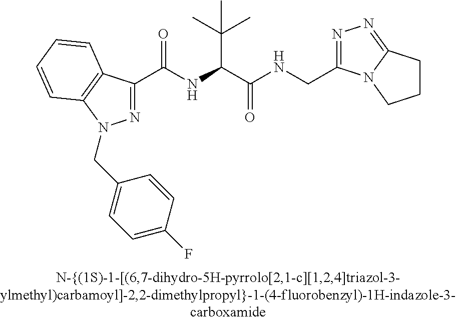 | ¹H NMR (400 MHz, CDCl3-d6) δ ppm δ 1.100 (s, 9H) 2.624-2.660 (m, 2H) 2.868-2.906 (m, 2H) 3.980-4.032 (m, 2H) 4.510-4.604 (m, 3H) 5.581 (s, 1H) 6.982-7.025 (m, 2H) 7.176-7.249 (m, 2H) 7.345-7.379 (m, 2H) 7.487 (s, 1H) 7.761-7.693 (d, J = 12.8 Hz, 1H) 8.240-8.261 (d, J = 8.4 Hz, 1H) | 504 |
| 420 | 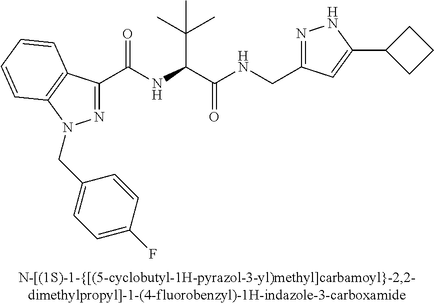 | ¹H NMR (400 MHz, CDCl3-d6) δ ppm δ 1.092 (s, 9H) 1.871-1.891 (m, 1H) 1.899-2.018 (m, 1H) 2.171-2.238 (m, 2H) 2.294-2.350 (m, 2H) 3.515-3.558 (m, 1H) 4.391-4.401 (m, 1H) 4.497-4.512 (m, 1H) 4.737-4.763 (d, J = 10.4 Hz, 1H) 5.594 (s, 2H) 6.022 (s, 1H) 6.938-7.026 (m, 2H) 7.194-7.271 (m, 2H) 7.300-7.378 (m, 2H) 7.818-7.843 (d, J = 10 Hz, 1H) 8.324-8.345 (d, J = 8.4 Hz, 1H), δ 8.456 (s, 1H) | 517 |
| 421 | 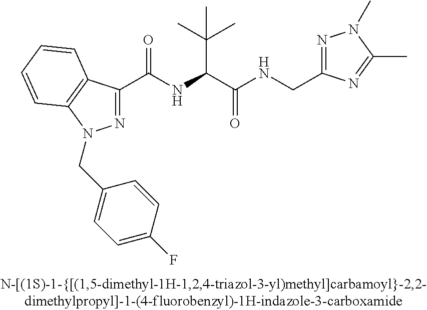 | ¹H NMR (400 MHz, CDCl3-d6) δ ppm δ 1.115 (s, 9H) 2.209 (s, 3H) 3.715 (s, 3H) 4.400-4.413 (d, 2H) 4.473-4.497 (d, J = 9.6 Hz, 1H) 5.586 (s, 2H) 5.930 (s, 1H) 6.349 (s, 1H) 6.981-7.024 (m, 2H) 7.192-7.236 (m, 2H) 7.273-7.291 (m, 1H) 7.312-7.357 (m, 1H) 7.740-7.763 (d, J = 9.2 Hz, 1H) 8.330-8.350 (d, J = 8 Hz, 1H) | 492 |
| 422 | 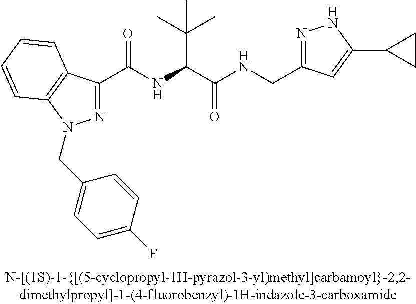 | ¹H NMR (400 MHz, CDCl3-d6) δ ppm δ 0.723-0.762 (m, 2H) 0.899-0.945 (m, 2H) 1.095 (s, 9H) 1.857-1.899 (m, 1H) 4.319-4.369 (m, 1H) 4.455-4.508 (m, 1H) 4.644-4.669 (d, J = 10 Hz, 1H) 5.593 (s, 2H) 5.856 (s, 1H) 6.986-7.029 (t, J = 12 Hz, 2H) 7.192-7.235 (m, 2H) 7.299-7.378 (m, 2H) 7.783-7.808 (d, J = 10 Hz, 1H) 7.973 (s, 1H) 8.323-8.344 (d, J = 8.4 Hz, 1H) | 503 |
| 423 | 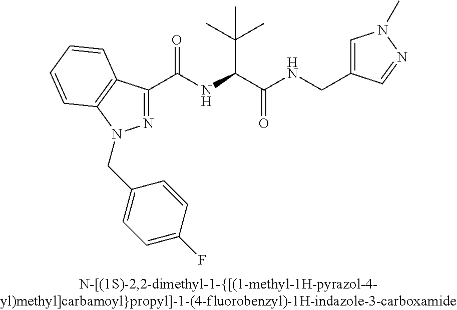 | ¹H NMR (400 MHz, CDCl3-d6) δ ppm 1.111 (s, 9H) 3.825 (s, 3H) 4.229-4.279 (m, 1H) 4.330-4.381 (m, 1H) 4.420-4.444 (d, J = 10.6 Hz, 1H) 5.591 (s, 2H) 6.183 (s, 1H) 6.987-7.030 (t, J = 16.4 Hz 2H) 7.192-7.227 (m, 2H) 7.274-7.324 (m, 2H) 7.351-7.387 (m, 2H) 7.704-7.728 (d, J = 10.6 Hz, 1H), 5 8.310-8.330 (d, J = 8 Hz, 1H) | 477 |
| 424 | 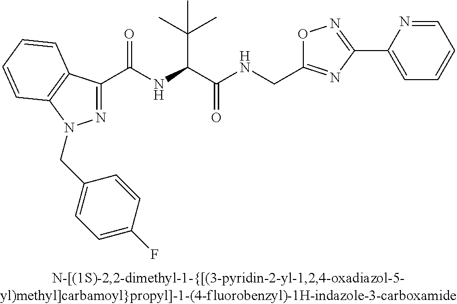 | ¹H NMR (400 MHz, CDCl3-d6) δ ppm 1.194 (s, 9H) 4.681-4.858 (m, 3H) 5.557 (s, 2H) 6.966-7.010 (t, J = 12 Hz, 2H) 7.168-7.259 (m, 4H) 7.263-7.339 (m, 3H) 8.310-8.330 (d, J = 8 Hz 1H) | 542 |
| 425 | 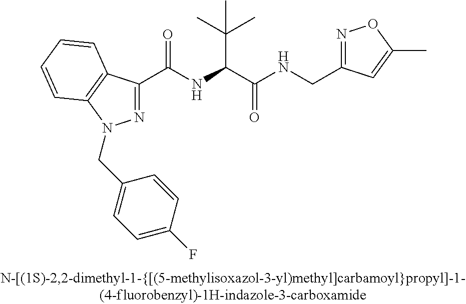 | ¹H NMR (400 MHz, CDCl3-d6) δ ppm 1.125 (s, 9H) 2.364 (s, 9H) 4.465-4.498 (m, 3H) 5.592 (s, 1H) 5.956 (s, 1H) 6.434 (s, 1H) 6.987-7.030 (m, 3H) 7.191-7.225 (m, 2H) 7.288-7.370 (m, 2H) 7.678-7.701 (d, J = 9.2 Hz, 1H) 8.323-8.343 (d, J = 8 Hz, 1H) | 478 |
| 426 | 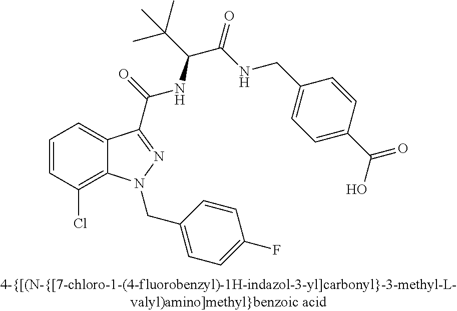 | ¹H NMR (400 MHz, DMSO-d6) δ ppm 8.90 (t, J = 5.86 Hz, 1H), 8.21 (d, J = 8.05 Hz, 1H), 7.87 (d, J = 8.05 Hz, 2H), 7.69 (d, J = 9.88 Hz, 1H), 7.55 (d, J = 7.32 Hz, 1H), 7.38 (d, J = 8.42 Hz, 2H), 7.29 (t, J = 7.87 Hz, 1H), 7.10-7.17 (m, 4H), 6.00-6.06 (m, 2H), 4.55 (d, J = 9.52 Hz, 1H), 4.28-4.45 (m, 2H), 0.96 (s, 9H) | 551 |
| 427 | 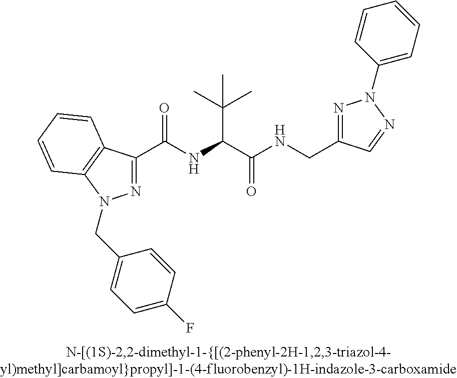 | ¹H NMR (400 MHz, CDCl3-d6) δ ppm 1.143 (s, 9H) 4.538-4.594 (m, 2H) 4.677-4.731 (m, 1H) 5.579 (s, 2H) 6.599-6.627 (m, 2H) 6.976-7.253 (m, 3H) 7.270-7.451 (m, 5H) 7.747-7.774 (d, J = 10.8 Hz, 2H) 7.993-7.996 (d, J = 1.2 Hz, 2H) 8.318-8.339 (d, J = 8.4 Hz, 1H) | 540 |
| 428 | 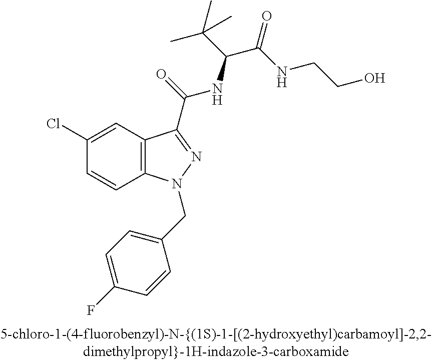 | ¹H NMR (400 MHz, DMSO-d6) δ ppm 0.95 (s, 9H) 3.12 (d, J = 5.86 Hz, 1H) 3.18 (d, J = 5.86 Hz, 1H) 3.21 (br. s., 1H) 3.40 (q, J = 5.86 Hz, 2H) 4.49 (d, J = 9.52 Hz, 1H) 4.67 (t, J = 5.31 Hz, 1H) 5.79 (s, 2H) 7.16 (t, J = 8.79 Hz, 2H) 7.31 (dd, J = 8.60, 5.67 Hz, 2H) 7.50 (dd, J = 8.97, 2.01 Hz, 1H) 7.60 (d, J = 9.88 Hz, 1H) 7.87 (d, J = 8.79 Hz, 1H) 8.13 (d, J = 1.83 Hz, 1H) 8.31 (t, J = 5.49 Hz, 1H) | 461 |
| 429 | 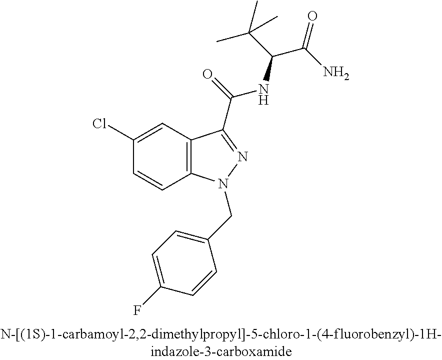 | ¹H NMR (400 MHz, DMSO-d6) δ ppm 0.97 (br. s., 8H) 3.31 (br. s., 3H) 4.45 (d, J = 9.15 Hz, 1H) 5.79 (br. s., 2H) 7.16 (t, J = 8.42 Hz, 2H) 7.24-7.34 (m, 3H) 7.50 (d, J = 8.79 Hz, 1H) 7.59 (d, J = 9.52 Hz, 1H) 7.74 (br. s., 1H) 7.87 (d, J = 8.79 Hz, 1H) 8.14 (br. s., 1H) | 417 |
| 430 | 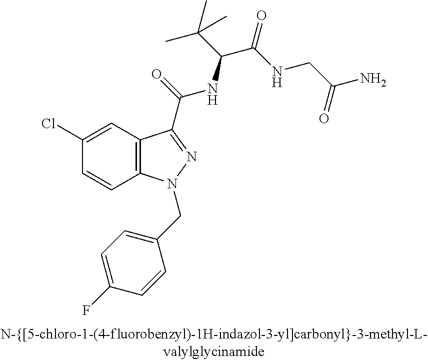 | ¹H NMR (400 MHz, DMSO-d6) δ ppm 0.84 (s, 1H) 0.98 (s, 9H) 3.67 (br. s., 1H) 3.68 (d, J = 4.39 Hz, 1H) 4.53 (d, J = 9.52 Hz, 1H) 5.79 (s, 2H) 7.00 (br. s., 1H) 7.16 (t, J = 8.79 Hz, 2H) 7.31 (dd, J = 8.42, 5.49 Hz, 3H) 7.50 (dd, J = 9.15, 1.83 Hz, 1H) 7.63 (d, J = 9.52 Hz, 1H) 7.86 (d, J = 9.15 Hz, 1H) 8.13 (d, J = 1.46 Hz, 1H) 8.49 (t, J = 5.67 Hz, 1H) | 474 |
| 431 | 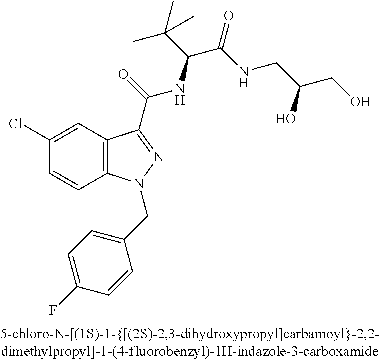 | ¹H NMR (400 MHz, DMSO-d6) δ ppm 0.96 (s, 10H) 2.96 (d, J = 6.95 Hz, 1H) 3.24-3.30 (m, 3H) 3.50 (d, J = 5.86 Hz, 1H) 4.50-4.58 (m, 2H) 4.72 (d, J = 4.76 Hz, 1H) 5.79 (s, 2H) 7.16 (t, J = 8.79 Hz, 2H) 7.31 (dd, J = 8.42, 5.49 Hz, 2H) 7.50 (dd, J = 8.97, 2.01 Hz, 1H) 7.62 (d, J = 9.52 Hz, 1H) 7.87 (d, J = 8.79 Hz, 1H) 8.13 (d, J = 1.83 Hz, 1H) 8.27 (t, J = 5.31 Hz, 1H) | 491 |
| 432 | 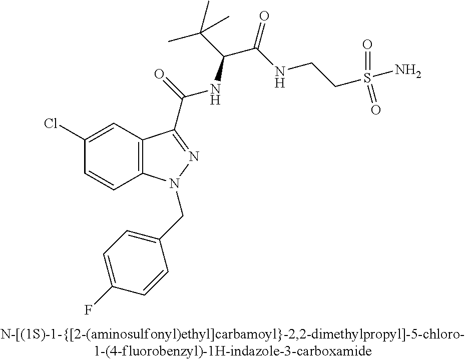 | ¹H NMR (400 MHz, DMSO-d6) δ ppm 0.96 (s, 9H) 3.07-3.18 (m, 2H) 3.38-3.46 (m, 1H) 3.50 (dd, J = 8.42, 5.49 Hz, 1H) 4.43 (d, J = 9.52 Hz, 1H) 5.79 (s, 2H) 6.91 (s, 2H) 7.16 (t, J = 8.79 Hz, 2H) 7.32 (dd, J = 8.60, 5.67 Hz, 2H) 7.50 (dd, J = 9.15, 1.83 Hz, 1H) 7.59 (d, J = 9.52 Hz, 1H) 7.87 (d, J = 8.79 Hz, 1H) 8.12 (d, J = 1.83 Hz, 1H) 8.52 (t, J = 5.49 Hz, 1H) | 525 |
| 433 | 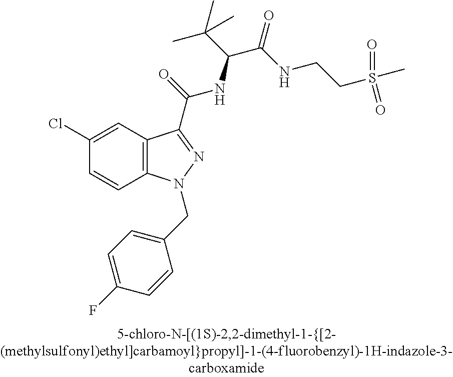 | ¹H NMR (400 MHz, DMSO-d6) δ ppm 0.96 (s, 9H) 2.99 (s, 3H) 3.27 (q, J = 6.83 Hz, 2H) 3.43-3.55 (m, 2H) 4.46 (d, J = 9.88 Hz, 1H) 5.79 (s, 2H) 7.16 (t, J = 8.79 Hz, 2H) 7.31 (dd, J = 8.60, 5.67 Hz, 2H) 7.50 (dd, J = 8.79, 1.83 Hz, 1H) 7.60 (d, J = 9.52 Hz, 1H) 7.87 (d, J = 8.79 Hz, 1H) 8.12 (d, J = 1.83 Hz, 1H) 8.61 (t, J = 5.49 Hz, 1H) | 524 |
| 434 | 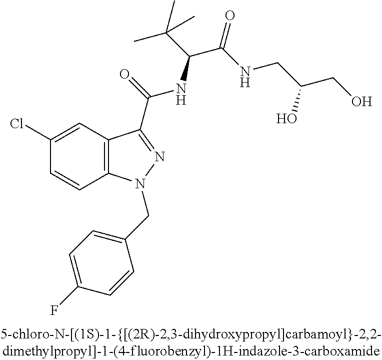 | ¹H NMR (400 MHz, DMSO-d6) δ ppm 0.96 (s, 9H) 3.02-3.12 (m, 1H) 3.13-3.23 (m, 1H) 3.28 (t, J = 5.49 Hz, 2H) 3.48 (d, J = 5.86 Hz, 1H) 4.49-4.57 (m, 2H) 4.74 (d, J = 5.12 Hz, 1H) 5.79 (s, 2H) 7.16 (t, J = 8.79 Hz, 2H) 7.31 (dd, J = 8.60, 5.67 Hz, 2H) 7.50 (dd, J = 8.79, 1.83 Hz, 1H) 7.61 (d, J = 9.52 Hz, 1H) 7.87 (d, J = 9.15 Hz, 1H) 8.13 (d, J = 1.83 Hz, 1H) 8.28 (t, J = 5.67 Hz, 1H) | 491 |
| 435 | 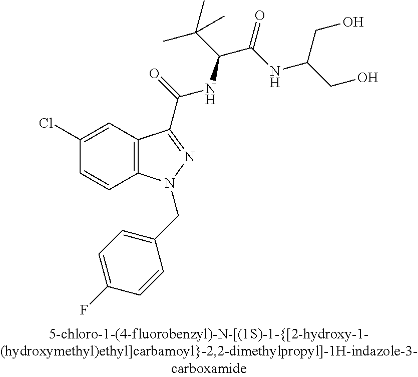 | ¹H NMR (400 MHz, DMSO-d6) δ ppm 0.95 (s, 9H) 3.36-3.47 (m, 4H) 3.76 (d, J = 7.32 Hz, 1H) 4.55 (d, J = 9.88 Hz, 1H) 4.63 (dt, J = 10.16, 5.35 Hz, 2H) 5.79 (s, 2H) 7.16 (t, J = 8.97 Hz, 2H) 7.31 (dd, J = 8.42, 5.49 Hz, 2H) 7.50 (dd, J = 9.15, 1.83 Hz, 1H) 7.61 (d, J = 9.52 Hz, 1H) 7.86 (d, J = 9.15 Hz, 1H) 8.06 (d, J = 8.05 Hz, 1H) 8.14 (d, J = 1.46 Hz, 1H) | 491 |
| 436 | 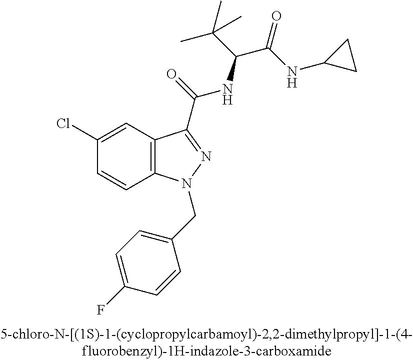 | ¹H NMR (400 MHz, DMSO-d6) δ ppm 0.34-0.41 (m, 2H) 0.62 (d, J = 6.95 Hz, 2H) 0.94 (s, 9H) 2.65 (dd, J = 7.14, 3.48 Hz, 1H) 4.39 (d, J = 9.52 Hz, 1H) 5.79 (s, 2H) 7.16 (t, J = 8.79 Hz, 2H) 7.32 (dd, J = 8.60, 5.67 Hz, 2H) 7.50 (dd, J = 9.15, 1.83 Hz, 1H) 7.56 (d, J = 9.52 Hz, 1H) 7.87 (d, J = 9.15 Hz, 1H) 8.12 (d, J = 1.83 Hz, 1H) 8.36 (d, J = 4.03 Hz, 1H) | 457 |
| 437 | 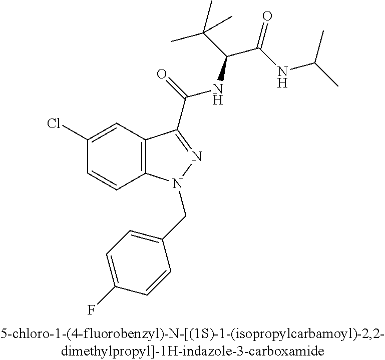 | ¹H NMR (400 MHz, DMSO-d6) δ ppm 0.95 (s, 9H) 1.05 (dd, J = 6.59, 3.29 Hz, 6H) 3.86 (d, J = 6.95 Hz, 1H) 4.44 (d, J = 9.52 Hz, 1H) 5.79 (s, 2H) 7.16 (t, J = 8.79 Hz, 2H) 7.31 (dd, J = 8.42, 5.49 Hz, 2H) 7.50 (dd, J = 9.15, 1.83 Hz, 1H) 7.57 (d, J = 9.52 Hz, 1H) 7.87 (d, J = 8.79 Hz, 1H) 8.13 (d, J = 1.46 Hz, 1H) 8.18 (d, J = 7.32 Hz, 1H) | 459 |
| 438 | 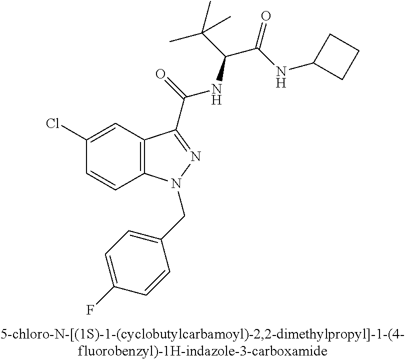 | ¹H NMR (400 MHz, DMSO-d6) δ ppm 0.95 (s, 9H) 1.56-1.67 (m, 2H) 1.82-1.93 (m, 2H) 2.08-2.19 (m, 2H) 4.19 (d, J = 8.05 Hz, 1H) 4.42 (d, J = 9.52 Hz, 1H) 5.79 (s, 2H) 7.16 (t, J = 8.79 Hz, 2H) 7.31 (dd, J = 8.42, 5.49 Hz, 2H) 7.50 (dd, J = 8.97, 2.01 Hz, 1H) 7.55 (d, J = 9.52 Hz, 1H) 7.86 (d, J = 9.15 Hz, 1H) 8.13 (d, J = 1.83 Hz, 1H) 8.54 (d, J = 7.69 Hz, 1H) | 471 |
| 439 | 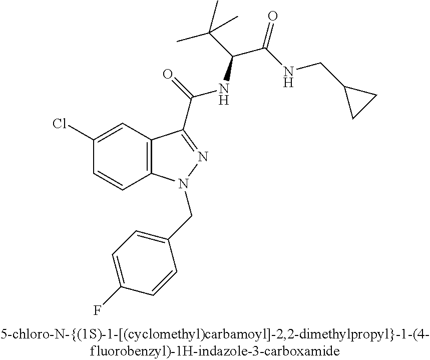 | ¹H NMR (400 MHz, DMSO-d6) δ ppm 0.14 (q, J = 4.64 Hz, 2H) 0.39 (q, J = 5.86 Hz, 2H) 0.88-0.99 (m, 10H) 2.84-2.93 (m, 1H) 2.99-3.08 (m, 1H) 4.48 (d, J = 9.52 Hz, 1H) 5.79 (s, 2H) 7.16 (t, J = 8.97 Hz, 2H) 7.31 (dd, J = 8.60, 5.67 Hz, 2H) 7.50 (dd, J = 8.97, 2.01 Hz, 1H) 7.59 (d, J = 9.52 Hz, 1H) 7.87 (d, J = 8.79 Hz, 1H) 8.13 (d, J = 1.46 Hz, 1H) 8.40 (t, J = 5.49 Hz, 1H) | 471 |
| 440 | 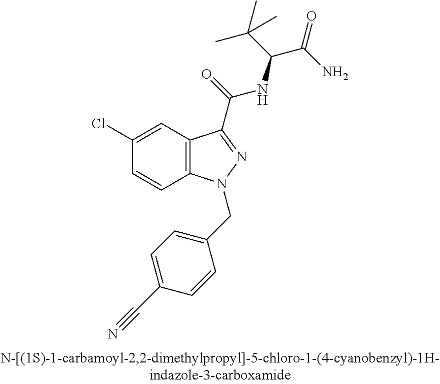 | ¹H NMR (400 MHz, DMSO-d6) δ ppm 0.97 (s, 9H) 4.45 (d, J = 9.52 Hz, 1H) 5.93 (d, J = 1.83 Hz, 2H) 7.29 (s, 1H) 7.35 (m, J = 8.05 Hz, 2H) 7.51 (dd, J = 9.15, 1.83 Hz, 1H) 7.59 (d, J = 9.52 Hz, 1H) 7.73 (br. s., 1H) 7.80 (m, J = 8.05 Hz, 2H) 7.84 (d, J = 8.79 Hz, 1H) 8.15 (d, J = 1.46 Hz, 1H) | 424 |
| 441 | 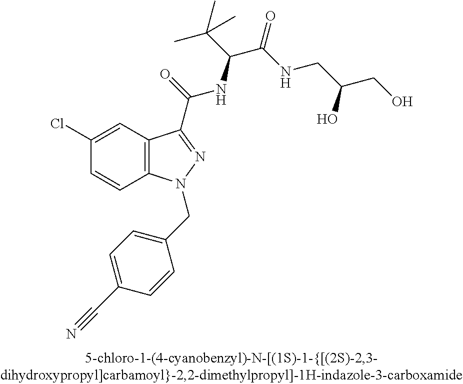 | ¹H NMR (400 MHz, DMSO-d6) δ ppm 0.96 (s, 9H) 2.95 (d, J = 6.95 Hz, 1H) 3.23-3.31 (m, 3H) 3.50 (d, J = 5.86 Hz, 1H) 4.48-4.58 (m, 2H) 4.72 (d, J = 5.12 Hz, 1H) 5.93 (s, 2H) 7.35 (m, J = 8.05 Hz, 2H) 7.51 (dd, J = 8.97, 2.01 Hz, 1H) 7.61 (d, J = 9.52 Hz, 1H) 7.80 (m, J = 8.42 Hz, 2H) 7.84 (d, J = 8.79 Hz, 1H) 8.15 (d, J = 1.83 Hz, 1H) 8.27 (t, J = 5.49 Hz, 1H) | 498 |
| 442 | 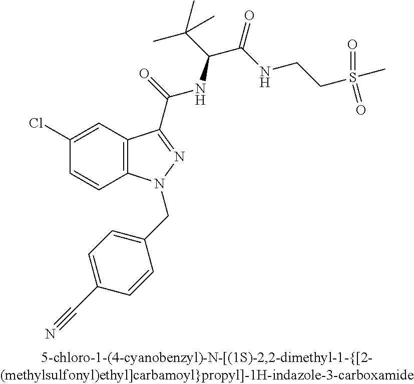 | ¹H NMR (400 MHz, DMSO-d6) δ ppm 0.96 (s, 9H) 3.00 (s, 3H) 3.28 (q, J = 6.95 Hz, 2H) 3.44-3.56 (m, 2H) 4.47 (d, J = 9.52 Hz, 1H) 5.93 (s, 2H) 7.36 (d, J = 8.05 Hz, 2H) 7.48 (s, 1H) 7.51 (d, J = 1.46 Hz, 1H) 7.61 (d, J = 9.88 Hz, 1H) 7.77-7.86 (m, 3H) 8.14 (s, 1H) 8.62 (t, J = 5.31 Hz, 1H) | 531 |
| 443 | 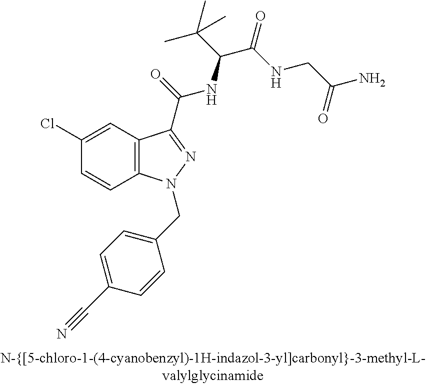 | ¹H NMR (400 MHz, DMSO-d6) δ ppm 0.97 (s, 9H) 3.68 (dd, J = 5.12, 3.66 Hz, 2H) 4.53 (d, J = 9.15 Hz, 1H) 5.93 (s, 2H) 7.00 (br. s., 1H) 7.33 (br. s., 1H) 7.35 (d, J = 8.05 Hz, 2H) 7.51 (dd, J = 9.15, 1.83 Hz, 1H) 7.62 (d, J = 9.52 Hz, 1H) 7.80 (d, J = 8.05 Hz, 2H) 7.84 (d, J = 9.15 Hz, 1H) 8.15 (d, J = 1.46 Hz, 1H) 8.49 (t, J = 5.67 Hz, 1H) | 481 |
| 444 | 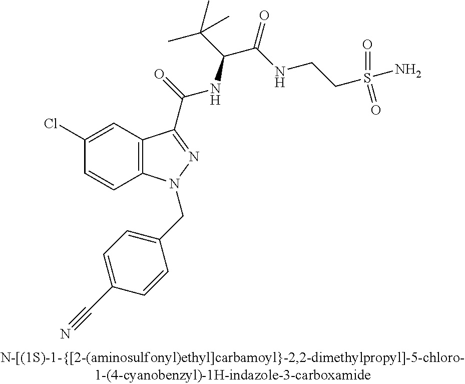 | ¹H NMR (400 MHz, DMSO-d6) δ ppm 0.96 (s, 9H) 3.07-3.18 (m, 2H) 3.38-3.46 (m, 1H) 3.50 (dd, J = 8.42, 5.49 Hz, 1H) 4.44 (d, J = 9.52 Hz, 1H) 5.93 (s, 2H) 6.91 (s, 2H) 7.36 (m, J = 8.42 Hz, 2H) 7.51 (dd, J = 8.97, 2.01 Hz, 1H) 7.59 (d, J = 9.52 Hz, 1H) 7.80 (m, J = 8.05 Hz, 2H) 7.85 (d, J = 9.15 Hz, 1H) 8.14 (d, J = 1.46 Hz, 1H) 8.52 (t, J = 5.49 Hz, 1H) | 532 |
| 445 | 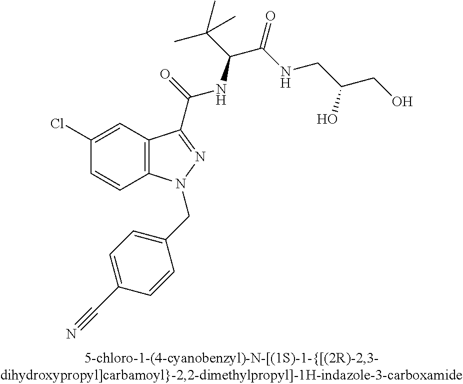 | ¹H NMR (400 MHz, DMSO-d6) δ ppm 0.95 (s, 9H) 3.01-3.11 (m, 1H) 3.13-3.23 (m, 1H) 3.28 (t, J = 5.67 Hz, 2H) 3.48 (d, J = 5.86 Hz, 1H) 4.50-4.57 (m, 2H) 4.74 (d, J = 4.76 Hz, 1H) 5.93 (s, 2H) 7.35 (m, J = 8.05 Hz, 2H) 7.51 (dd, J = 9.15, 1.83 Hz, 1H) 7.60 (d, J = 9.88 Hz, 1H) 7.80 (m, J = 8.05 Hz, 2H) 7.84 (d, J = 8.79 Hz, 1H) 8.15 (d, J = 1.46 Hz, 1H) 8.28 (t, J = 5.67 Hz, 1H) | 498 |
| 446 | 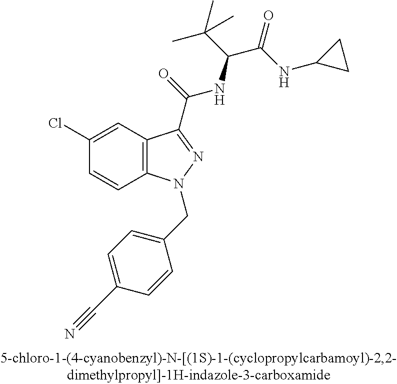 | ¹H NMR (400 MHz, DMSO-d6) δ ppm 0.34-0.45 (m, 2H) 0.57-0.66 (m, 2H) 0.93 (s, 9H) 0.97 (br. s., 1H) 2.65 (dd, J = 7.32, 3.29 Hz, 1H) 4.39 (d, J = 9.88 Hz, 1H) 5.94 (d, J = 1.83 Hz, 2H) 7.36 (m, J = 8.05 Hz, 2H) 7.51 (dd, J = 8.79, 1.83 Hz, 1H) 7.56 (d, J = 9.52 Hz, 1H) 7.80 (m, J = 8.42 Hz, 2H) 7.85 (d, J = 9.15 Hz, 1H) 8.14 (d, J = 1.46 Hz, 1H) 8.36 (d, J = 4.03 Hz, 1H) | 464 |
| 447 | 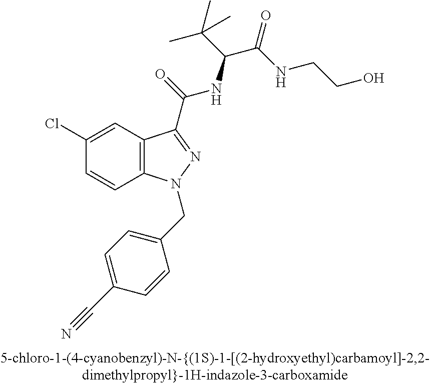 | ¹H NMR (400 MHz, DMSO-d6) δ ppm 0.95 (s, 9H) 3.12 (d, J = 5.86 Hz, 1H) 3.18 (d, J = 5.86 Hz, 1H) 3.40 (q, J = 5.86 Hz, 2H) 4.49 (d, J = 9.52 Hz, 1H) 4.67 (t, J = 5.12 Hz, 1H) 5.93 (s, 2H) 7.35 (m, J = 8.42 Hz, 2H) 7.51 (dd, J = 8.97, 2.01 Hz, 1H) 7.59 (d, J = 9.52 Hz, 1H) 7.80 (m, J = 8.42 Hz, 2H) 7.84 (d, J = 8.79 Hz, 1H) 8.15 (d, J = 1.83 Hz, 1H) 8.31 (t, J = 5.49 Hz, 1H) | 468 |
| 448 | 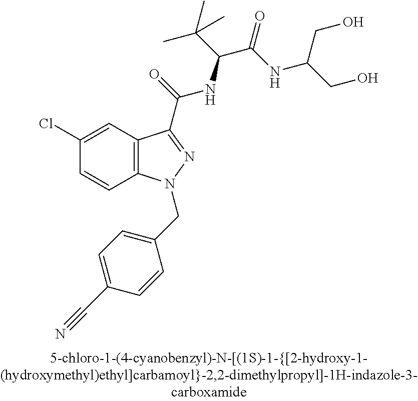 | ¹H NMR (400 MHz, DMSO-d6) δ ppm 0.95 (s, 9H) 3.36-3.47 (m, 3H) 3.76 (d, J = 7.32 Hz, 1H) 4.55 (d, J = 9.52 Hz, 1H) 4.59-4.66 (m, 2H) 5.93 (s, 2H) 7.35 (d, J = 8.05 Hz, 2H) 7.51 (dd, J = 8.79, 1.83 Hz, 1H) 7.60 (d, J = 9.52 Hz, 1H) 7.77-7.86 (m, 3H) 8.06 (d, J = 8.05 Hz, 1H) 8.16 (d, J = 1.83 Hz, 1H) | 498 |
| 449 | 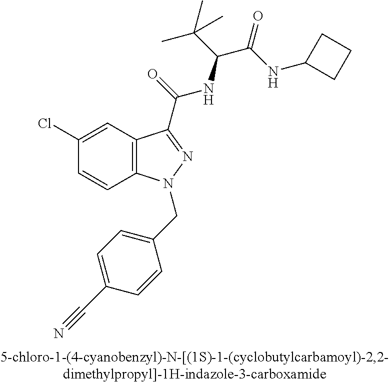 | ¹H NMR (400 MHz, DMSO-d6) δ ppm 0.94 (s, 9H) 1.56-1.67 (m, 2H) 1.81-1.92 (m, 2H) 2.08-2.19 (m, 2H) 4.18 (d, J = 8.05 Hz, 1H) 4.42 (d, J = 9.52 Hz, 1H) 5.93 (d, J = 2.20 Hz, 2H) 7.35 (m, J = 8.42 Hz, 2H) 7.48-7.58 (m, 2H) 7.80 (m, J = 8.05 Hz, 2H) 7.84 (d, J = 9.15 Hz, 1H) 8.15 (d, J = 1.46 Hz, 1H) 8.54 (d, J = 7.69 Hz, 1H) | 479 |
| 450 | 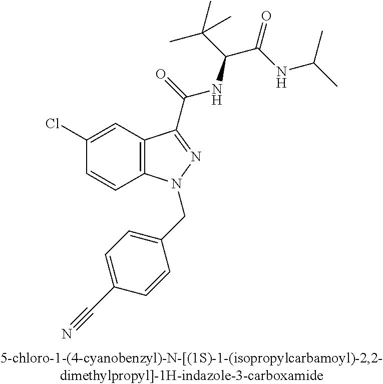 | ¹H NMR (400 MHz, DMSO-d6) δ ppm 0.95 (s, 9H) 1.05 (dd, J = 6.41, 2.75 Hz, 6H) 3.80-3.90 (m, 1H) 4.44 (d, J = 9.88 Hz, 1H) 5.93 (s, 2H) 7.35 (m, J = 8.05 Hz, 2H) 7.51 (dd, J = 8.97, 2.01 Hz, 1H) 7.57 (d, J = 9.52 Hz, 1H) 7.80 (m, J = 8.42 Hz, 2H) 7.85 (d, J = 9.15 Hz, 1H) 8.15 (d, J = 1.46 Hz, 1H) 8.18 (d, J = 7.69 Hz, 1H) | 466 |
| 451 | 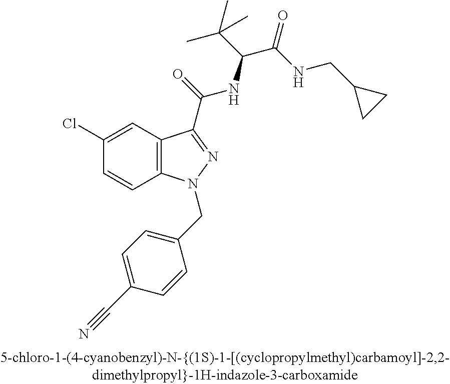 | ¹H NMR (400 MHz, DMSO-d6) δ ppm 0.14 (q, J = 4.64 Hz, 2H) 0.34-0.43 (m, 2H) 0.87-0.98 (m, 10H) 2.83-2.92 (m, 1H) 2.99-3.08 (m, 1H) 4.49 (d, J = 9.52 Hz, 1H) 5.93 (d, J = 2.20 Hz, 2H) 7.35 (m, J = 8.05 Hz, 2H) 7.51 (dd, J = 8.97, 2.01 Hz, 1H) 7.59 (d, J = 9.52 Hz, 1H) 7.80 (m, J = 8.42 Hz, 2H) 7.85 (d, J = 8.79 Hz, 1H) 8.15 (d, J = 1.83 Hz, 1H) 8.40 (t, J = 5.49 Hz, 1H) | 479 |
| 452 | 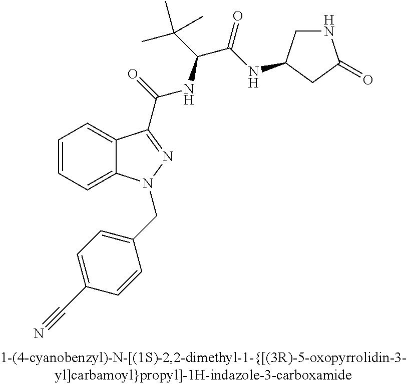 | ¹H NMR (400 MHz, DMSO-d6) δ ppm 8.74 (d, J = 6.59 Hz, 1H) 8.18 (d, J = 8.05 Hz, 1H) 7.74-7.83 (m, 3H) 7.66 (s, 1H) 7.60 (d, J = 9.52 Hz, 1H) 7.47 (t, J = 7.50 Hz, 1H) 7.26-7.39 (m, 3H) 5.92 (s, 2H) 4.48 (d, J = 9.88 Hz, 1H) 4.31-4.40 (m, 1H) 3.48 (dd, J = 10.07, 7.14 Hz, 1H) 3.02 (dd, J = 10.07, 3.48 Hz, 1H) 1.99 (dd, J = 16.66, 4.21 Hz, 1H) 0.95 (s, 9H). | 473 |
| 453 | 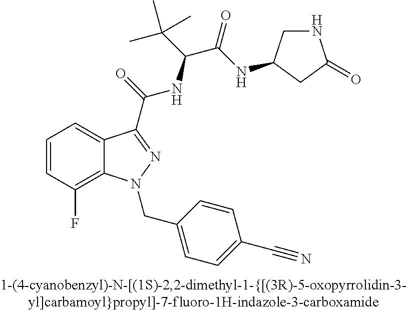 | ¹H NMR (400 MHz, DMSO-d6) δ ppm 8.74 (d, J = 6.59 Hz, 1H) 8.00 (d, J = 7.32 Hz, 1H) 7.81 (d, J = 8.05 Hz, 2H) 7.62-7.69 (m, 2H) 7.23-7.35 (m, 4H) 5.93 (s, 2H) 4.48 (d, J = 9.52 Hz, 1H) 4.32-4.39 (m, 1H) 3.48 (dd, J = 9.88, 6.95 Hz, 1H) 3.02 (dd, J = 10.07, 3.48 Hz, 1H) 1.99 (dd, J = 16.66, 4.21 Hz, 1H) 0.96 (s, 9H). | 491 |
| 454 | 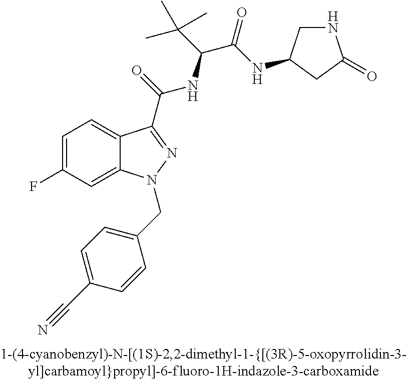 | ¹H NMR (400 MHz, DMSO-d6) δ ppm 8.73 (d, J = 6.22 Hz, 1H) 8.18 (dd, J = 8.79, 5.49 Hz, 1H) 7.73-7.84 (m, 3H) 7.66 (s, 1H) 7.58 (d, J = 9.88 Hz, 1H) 7.38 (d, J = 8.05 Hz, 2H) 7.20 (td, J = 9.15, 1.83 Hz, 1H) 5.87 (s, 2H) 4.46 (d, J = 9.88 Hz, 1H) 4.31-4.40 (m, 1H) 3.48 (dd, J = 9.88, 6.95 Hz, 1H) 3.01 (dd, J = 10.07, 3.48 Hz, 1H) 1.98 (dd, J = 16.84, 4.39 Hz, 1H) 0.94 (s, 9H). | 491 |
| 455 | 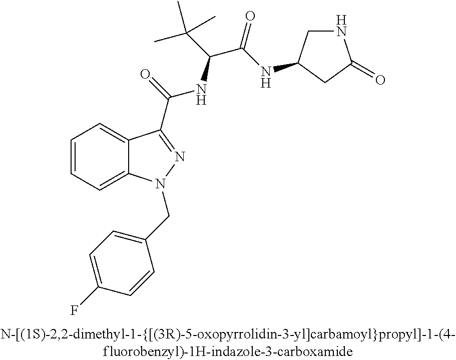 | ¹H NMR (400 MHz, DMSO-d6) δ ppm 8.74 (d, J = 6.59 Hz, 1H) 8.16 (d, J = 8.05 Hz, 1H) 7.79 (d, J = 8.42 Hz, 1H) 7.66 (s, 1H) 7.60 (d, J = 9.52 Hz, 1H) 7.45 (t, J = 7.50 Hz, 1H) 7.25-7.34 (m, 3H) 7.15 (t, J = 8.79 Hz, 2H) 5.78 (s, 2H) 4.48 (d, J = 9.52 Hz, 1H) 4.34-4.41 (m, 1H) 3.48 (dd, J = 10.07, 7.14 Hz, 1H) 3.02 (dd, J = 10.07, 3.48 Hz, 1H) 1.99 (dd, J = 16.84, 4.39 Hz, 1H) 0.96 (s, 9H). | 466 |
| 456 | 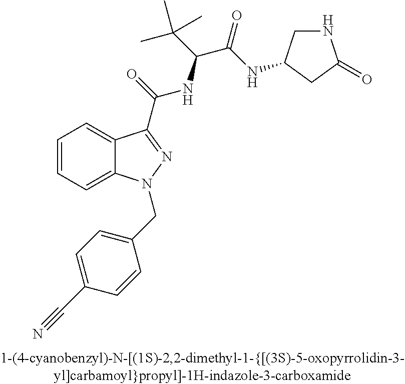 | ¹H NMR (400 MHz, DMSO-d6) δ ppm 8.74 (d, J = 6.59 Hz, 1H) 8.18 (d, J = 8.05 Hz, 1H) 7.74-7.85 (m, 3H) 7.66 (s, 1H) 7.59 (d, J = 9.52 Hz, 1H) 7.47 (t, J = 7.50 Hz, 1H) 7.27-7.39 (m, 3H) 5.92 (s, 2H) 4.48 (d, J = 9.52 Hz, 1H) 4.33-4.41 (m, 1H) 3.51 (dd, J = 9.88, 6.95 Hz, 1H) 3.00 (dd, J = 10.07, 3.48 Hz, 1H) 2.40-2.48 (m, 1H) 2.03 (dd, J = 16.84, 4.39 Hz, 1H) 0.96 (s, 9H). | 473 |
| 457 | 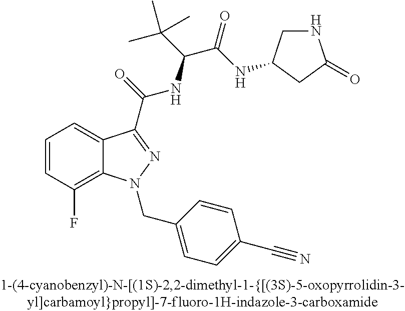 | ¹H NMR (400 MHz, DMSO-d6) δ ppm 8.75 (d, J = 6.59 Hz, 1H) 8.00 (d, J = 7.32 Hz, 1H) 7.80 (d, J = 8.42 Hz, 2H) 7.61-7.69 (m, 2H) 7.22-7.36 (m, 4H) 5.93 (s, 2H) 4.49 (d, J = 9.88 Hz, 1H) 4.33-4.42 (m, 1H) 3.52 (dd, J = 9.88, 6.95 Hz, 1H) 3.00 (dd, J = 10.07, 3.48 Hz, 1H) 2.42-2.47 (m, 1H) 2.03 (dd, J = 16.84, 4.39 Hz, 1H) 0.96 (s, 9H).. | 491 |
| 458 | 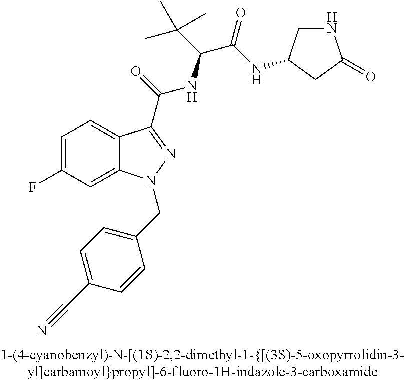 | ¹H NMR (400 MHz, DMSO-d6) δ ppm 8.73 (d, J = 6.22 Hz, 1H) 8.18 (dd, J = 8.79, 5.49 Hz, 1H) 7.81 (d, J = 8.05 Hz, 2H) 7.74 (dd, J = 9.70, 1.65 Hz, 1H) 7.66 (s, 1H) 7.57 (d, J = 9.88 Hz, 1H) 7.37 (d, J = 8.05 Hz, 2H) 7.20 (td, J = 9.15, 1.83 Hz, 1H) 5.87 (s, 2H) 4.46 (d, J = 9.52 Hz, 1H) 4.31-4.41 (m, 1H) 3.51 (dd, J = 9.88, 6.95 Hz, 1H) 2.99 (dd, J = 9.88, 3.66 Hz, 1H) 2.41-2.48 (m, 1H) 2.02 (dd, J = 16.84, 4.76 Hz, 1H) 0.95 (s, 9H). | 491 |
| 459 | 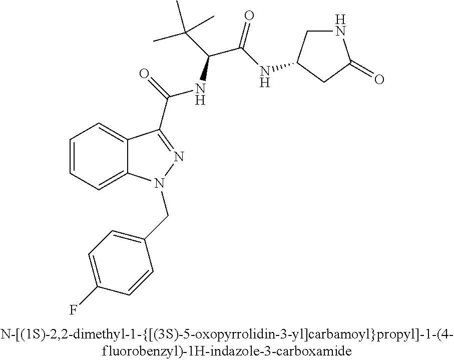 | ¹H NMR (400 MHz, DMSO-d6) δ ppm 8.74 (d, J = 6.59 Hz, 1H) 8.16 (d, J = 8.05 Hz, 1H) 7.79 (d, J = 8.79 Hz, 1H) 7.66 (s, 1H) 7.60 (d, J = 9.88 Hz, 1H) 7.45 (t, J = 7.50 Hz, 1H) 7.26-7.34 (m, 3H) 7.15 (t, J = 8.79 Hz, 2H) 5.77 (s, 2H) 4.48 (d, J = 9.88 Hz, 1H) 4.32-4.43 (m, 1H) 3.52 (dd, J = 9.88, 6.95 Hz, 1H) 3.00 (dd, J = 9.88, 3.66 Hz, 1H) 2.41-2.47 (m, 1H) 2.04 (dd, J = 16.84, 4.76 Hz, 1H) 0.96 (s, 9H). | 466 |
| 460 | 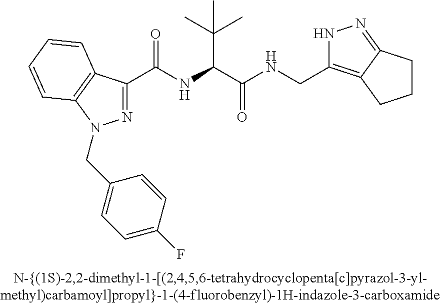 | ¹H NMR (400 MHz, CDCl3-d6) δ ppm 0.881-0.956 (m, 1H) 1.115 (s, 9H) 2.301-2.575 (m, 6H) 4.268-4.471 (m, 2H) 4.648-4.671 (m, 1H) 5.576 (s, 2H) 6.976-7.018 (m, 2H) 7.187-7.351 (m, 5H) 7.784-7.857 (m, 1H) 8.256-8.276 (m, 1H) | 503 |
| 461 | 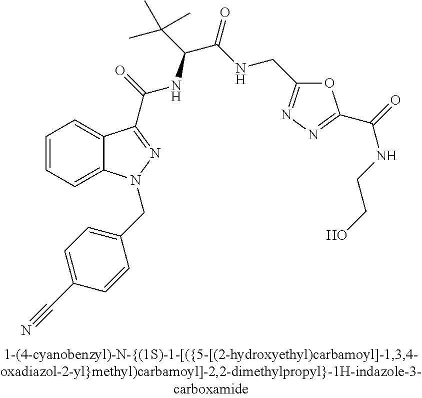 | ¹H NMR (400 MHz, DMSO-d6) δ ppm 9.14 (dt, J = 15.74, 5.49 Hz, 2H) 8.18 (d, J = 8.05 Hz, 1H) 7.73-7.82 (m, 2H) 7.62 (d, J = 9.52 Hz, 1H) 7.47 (t, J = 7.50 Hz, 1H) 7.27-7.38 (m, 3H) 5.91 (s, 2H) 4.77 (t, J = 5.67 Hz, 1H) 4.53-4.71 (m, 3H) 3.48 (q, J = 5.86 Hz, 2H) 3.22-3.32 (m, 2H) 0.98 (s, 9H). | 559 |
| 462 | 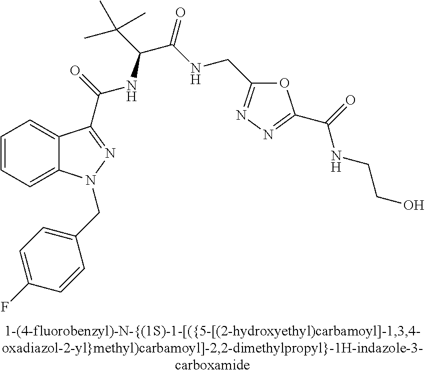 | ¹H NMR (400 MHz, DMSO-d6) δ ppm 9.15 (dt, J = 16.20, 5.63 Hz, 2H) 8.16 (d, J = 8.42 Hz, 1H) 7.78 (d, J = 8.79 Hz, 1H) 7.62 (d, J = 9.88 Hz, 1H) 7.45 (t, J = 7.69 Hz, 1H) 7.26-7.34 (m, 2H) 7.15 (t, J = 8.79 Hz, 2H) 5.77 (s, 1H) 4.77 (t, J = 5.67 Hz, 1H) 4.53-4.72 (m, 3H) 3.48 (q, J = 5.98 Hz, 2H) 3.24-3.33 (m, 2H) 0.98 (s, 9H). | 552 |
| 463 | 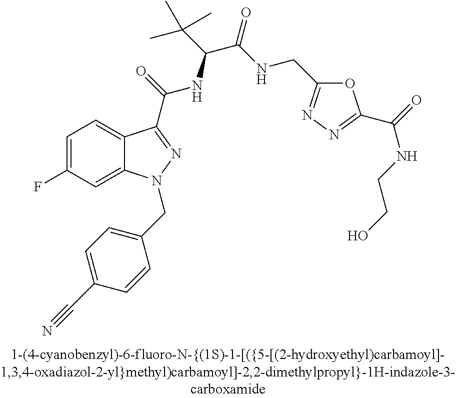 | ¹H NMR (400 MHz, DMSO-d6) δ ppm 9.09-9.19 (m, 2H) 8.18 (dd, J = 8.97, 5.31 Hz, 1H) 7.71-7.85 (m, 2H) 7.60 (d, J = 9.52 Hz, 1H) 7.37 (d, J = 8.05 Hz, 2H) 7.20 (t, J = 8.42 Hz, 1H) 5.86 (s, 2H) 4.77 (t, J = 5.67 Hz, 1H) 4.50-4.72 (m, 3H) 3.48 (q, J = 5.86 Hz, 2H) 3.19-3.31 (m, 2H) 0.97 (s, 9H). | 577 |
| 464 | 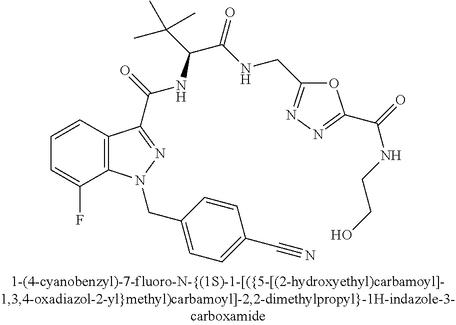 | ¹H NMR (400 MHz, DMSO-d6) δ ppm 9.15 (dt, J = 18.58, 5.54 Hz, 2H) 8.00 (d, J = 7.69 Hz, 1H) 7.80 (d, J = 8.05 Hz, 2H) 7.67 (d, J = 9.88 Hz, 1H) 7.22-7.35 (m, 3H) 5.93 (s, 2H) 4.77 (t, J = 5.49 Hz, 1H) 4.52-4.71 (m, 3H) 3.48 (q, J = 5.73 Hz, 2H) 3.25-3.33 (m, 2H) 0.98 (s, 9H). | 577 |
| 465 | 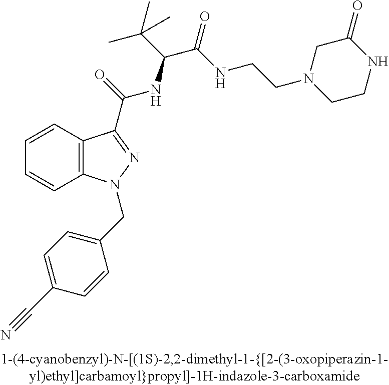 | ¹H NMR (400 MHz, DMSO-d6) δ ppm 0.90 (br. s., 1H) 0.96 (s, 10H) 2.56 (dd, J = 4.58, 2.38 Hz, 1H) 2.98 (s, 2H) 3.10 (br. s., 3H) 4.47 (d, J = 9.88 Hz, 1H) 5.91 (s, 2H) 7.27-7.37 (m, 3H) 7.46 (t, J = 7.69 Hz, 1H) 7.60 (d, J = 9.52 Hz, 1H) 7.76 (d, J = 8.79 Hz, 2H) 7.80 (d, J = 8.05 Hz, 2H) 8.18 (d, J = 8.42 Hz, 1H) 8.27 (br. s., 1H) | 516 |
| 466 | 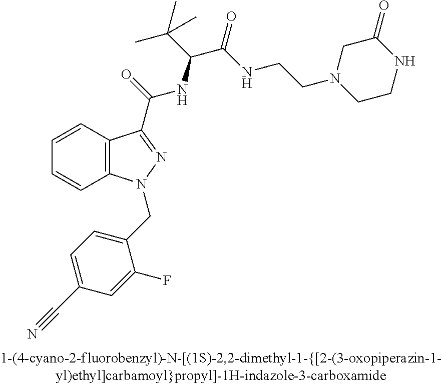 | ¹H NMR (400 MHz, DMSO-d6) δ ppm 0.95 (s, 9H) 2.41 (br. s., 2H) 2.92 (br. s., 1H) 2.96 (d, J = 13.54 Hz, 1H) 3.03-3.14 (m, 3H) 3.31 (br. s., 1H) 3.36 (s, 1H) 4.46 (d, J = 9.52 Hz, 1H) 5.94 (s, 2H) 7.16 (t, J = 7.69 Hz, 1H) 7.31 (t, J = 7.50 Hz, 1H) 7.48 (t, J = 7.69 Hz, 1H) 7.56 (d, J = 9.52 Hz, 1H) 7.63 (d, J = 8.05 Hz, 1H) 7.71 (br. s., 1H) 7.78 (d, J = 8.42 Hz, 1H) 7.91 (d, J = 9.88 Hz, 1H) 8.18 (d, J = 8.42 Hz, 1H) 8.26 (t, J = 5.31 Hz, 1H) | 534 |
| 467 | 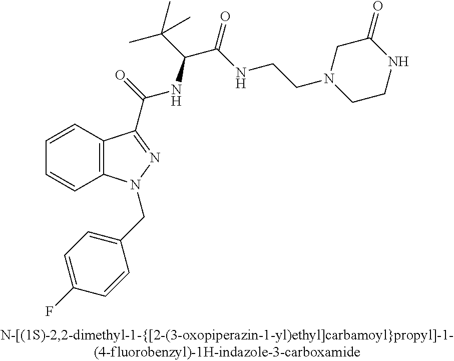 | ¹H NMR (400 MHz, DMSO-d6) δ ppm 0.96 (s, 10H) 2.37-2.48 (m, 3H) 2.54 (br. s., 1H) 2.93 (d, J = 11.71 Hz, 2H) 3.06 (br. s., 1H) 3.09 (t, J = 5.31 Hz, 3H) 4.47 (d, J = 9.52 Hz, 1H) 5.77 (s, 2H) 7.15 (t, J = 8.79 Hz, 2H) 7.25-7.34 (m, 3H) 7.45 (t, J = 7.69 Hz, 1H) 7.60 (d, J = 9.88 Hz, 1H) 7.70 (br. s., 1H) 7.78 (d, J = 8.79 Hz, 1H) 8.16 (d, J = 8.42 Hz, 1H) 8.26 (t, J = 5.49 Hz, 1H) | 509 |
| 468 | 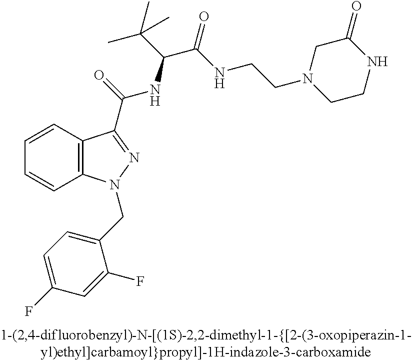 | ¹H NMR (400 MHz, DMSO-d6) δ ppm 0.89 (s, 1H) 0.95 (s, 10H) 2.37-2.48 (m, 3H) 2.93 (d, J = 11.71 Hz, 2H) 3.03-3.14 (m, 3H) 4.45 (d, J = 9.52 Hz, 1H) 5.80 (s, 2H) 7.02-7.07 (m, 1H) 7.22-7.32 (m, 3H) 7.47 (t, J = 7.69 Hz, 1H) 7.56 (d, J = 9.52 Hz, 1H) 7.70 (br. s., 1H) 7.78 (d, J = 8.42 Hz, 1H) 8.16 (d, J = 8.42 Hz, 1H) 8.26 (t, J = 5.49 Hz, 1H) | 527 |
| 469 | 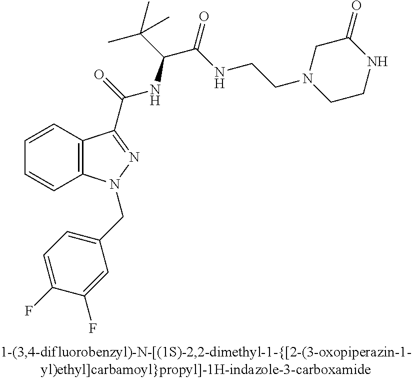 | ¹H NMR (400 MHz, DMSO-d6) δ ppm 0.96 (s, 10H) 2.41 (d, J = 5.49 Hz, 3H) 2.54 (br. s., 1H) 2.93 (d, J = 12.08 Hz, 2H) 3.04-3.14 (m, 3H) 4.47 (d, J = 9.52 Hz, 1H) 5.78 (s, 2H) 7.04 (td, J = 4.21, 2.20 Hz, 1H) 7.29 (t, J = 7.50 Hz, 1H) 7.34-7.43 (m, 2H) 7.46 (t, J = 7.69 Hz, 1H) 7.61 (d, J = 9.88 Hz, 1H) 7.70 (br. s., 1H) 7.80 (d, J = 8.79 Hz, 1H) 8.16 (d, J = 8.05 Hz, 1H) 8.26 (t, J = 5.49 Hz, 1H) | 527 |
| 470 | 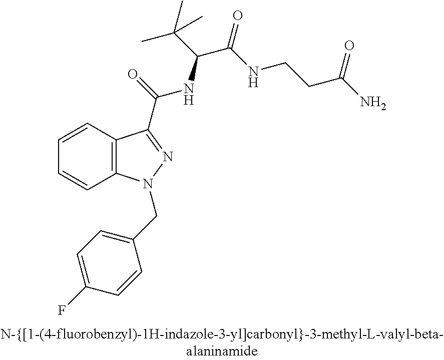 | ¹H NMR (400 MHz, DMSO-d6) δ ppm 0.95 (s, 9H) 2.25 (t, J = 7.14 Hz, 2H) 3.14-3.25 (m, 1H) 4.46 (d, J = 9.88 Hz, 1H) 5.77 (s, 2H) 6.83 (br. s., 1H) 7.15 (t, J = 8.79 Hz, 2H) 7.25-7.35 (m, 4H) 7.45 (t, J = 7.69 Hz, 1H) 7.60 (d, J = 9.52 Hz, 1H) 7.78 (d, J = 8.42 Hz, 1H) 8.16 (d, J = 8.42 Hz, 1H) 8.34 (t, J = 5.49 Hz, 1H) | 454 |
| 471 | 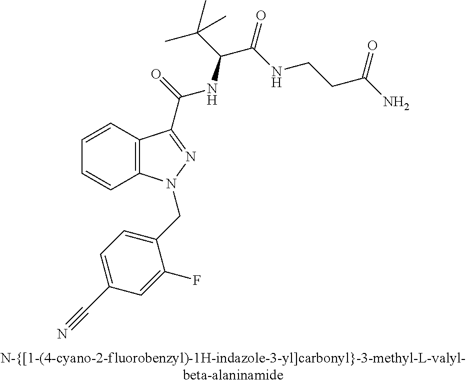 | ¹H NMR (400 MHz, DMSO-d6) δ ppm 0.89-0.98 (m, 9H) 2.23 (t, J = 7.14 Hz, 2H) 3.14-3.24 (m, 1H) 3.29 (d, J = 6.22 Hz, 1H) 4.45 (d, J = 9.88 Hz, 1H) 5.94 (s, 2H) 6.82 (br. s., 1H) 7.15 (t, J = 7.69 Hz, 1H) 7.31 (t, J = 7.32 Hz, 2H) 7.48 (t, J = 7.32 Hz, 1H) 7.55 (d, J = 9.88 Hz, 1H) 7.63 (d, J = 6.59 Hz, 1H) 7.78 (d, J = 8.79 Hz, 1H) 7.91 (d, J = 8.42 Hz, 1H) 8.18 (d, J = 8.05 Hz, 1H) 8.33 (t, J = 5.49 Hz, 1H) | 479 |
| 472 | 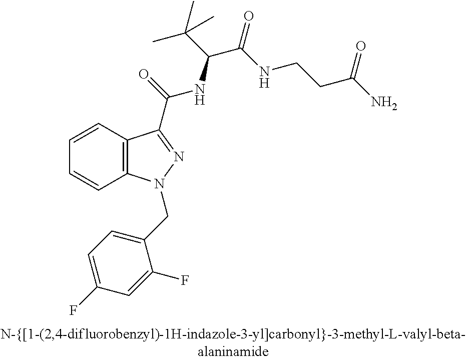 | ¹H NMR (400 MHz, DMSO-d6) δ ppm 0.89-0.98 (m, 9H) 2.24 (t, J = 7.14 Hz, 2H) 3.14-3.23 (m, 1H) 3.29 (d, J = 5.49 Hz, 1H) 4.44 (d, J = 9.52 Hz, 1H) 5.80 (s, 2H) 6.82 (br. s., 1H) 7.05 (d, J = 2.20 Hz, 1H) 7.22-7.33 (m, 4H) 7.47 (t, J = 7.69 Hz, 1H) 7.55 (d, J = 9.52 Hz, 1H) 7.78 (d, J = 8.42 Hz, 1H) 8.16 (d, J = 8.05 Hz, 1H) 8.33 (t, J = 5.49 Hz, 1H) | 472 |
| 473 | 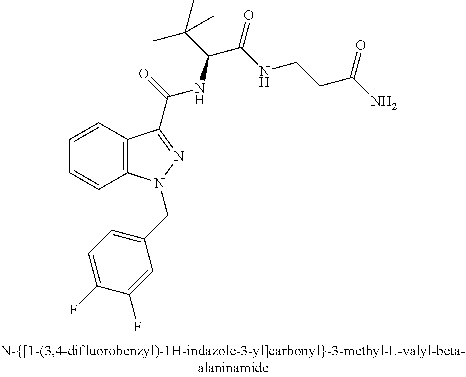 | ¹H NMR (400 MHz, DMSO-d6) δ ppm 0.95 (s, 10H) 2.25 (t, J = 7.14 Hz, 2H) 3.15-3.24 (m, 1H) 4.46 (d, J = 9.52 Hz, 1H) 5.78 (s, 2H) 6.82 (br. s., 1H) 7.04 (td, J = 4.21, 2.20 Hz, 1H) 7.27-7.33 (m, 2H) 7.35-7.42 (m, 2H) 7.46 (t, J = 7.50 Hz, 1H) 7.61 (d, J = 9.88 Hz, 1H) 7.80 (d, J = 8.42 Hz, 1H) 8.16 (d, J = 8.05 Hz, 1H) 8.34 (t, J = 5.49 Hz, 1H) | 472 |
| 474 | 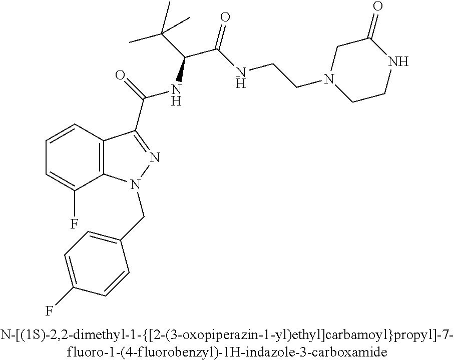 | ¹H NMR (400 MHz, DMSO-d6) δ ppm 0.92-1.00 (m, 10H) 2.37-2.48 (m, 3H) 2.54-2.59 (m, 1H) 2.93 (d, J = 11.35 Hz, 2H) 3.04-3.14 (m, 3H) 4.47 (d, J = 9.88 Hz, 1H) 5.79 (s, 2H) 7.13-7.19 (m, 2H) 7.21-7.32 (m, 4H) 7.64 (d, J = 9.52 Hz, 1H) 7.70 (s, 1H) 7.98 (d, J = 7.69 Hz, 1H) 8.27 (t, J = 5.49 Hz, 1H) | 527 |
| 475 | 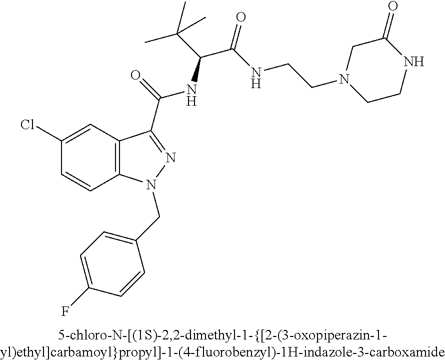 | ¹H NMR (400 MHz, DMSO-d6) δ ppm 0.95 (s, 9H) 2.37-2.48 (m, 2H) 2.51 (br. s., 1H) 2.55 (br. s., 1H) 2.92 (d, J = 12.08 Hz, 2H) 3.06 (br. s., 1H) 3.09 (t, J = 5.31 Hz, 3H) 3.32 (d, J = 2.20 Hz, 1H) 3.38 (s, 1H) 4.46 (d, J = 9.52 Hz, 1H) 5.78 (s, 2H) 7.15 (t, J = 8.79 Hz, 2H) 7.31 (dd, J = 8.60, 5.67 Hz, 2H) 7.49 (dd, J = 9.15, 1.83 Hz, 1H) 7.60 (d, J = 9.52 Hz, 1H) 7.70 (s, 1H) 7.86 (d, J = 9.15 Hz, 1H) 8.12 (d, J = 1.83 Hz, 1H) 8.27 (t, J = 5.49 Hz, 1H) | 544 |
| 476 | 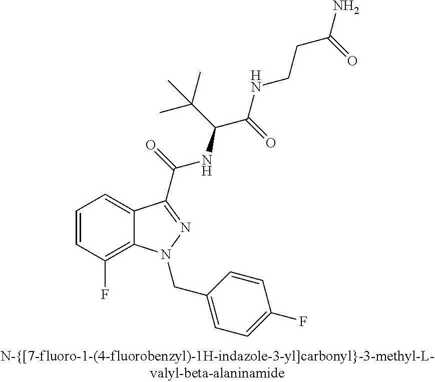 | ¹H NMR (400 MHz, DMSO-d6) δ ppm 0.95 (s, 10H) 2.25 (t, J = 7.14 Hz, 2H) 3.15-3.25 (m, 1H) 4.47 (d, J = 9.88 Hz, 1H) 5.79 (s, 2H) 6.82 (br. s., 1H) 7.13-7.19 (m, 2H) 7.21-7.33 (m, 5H) 7.64 (d, J = 9.52 Hz, 1H) 7.99 (d, J = 7.69 Hz, 1H) 8.35 (t, J = 5.49 Hz, 1H) | 472 |
| 477 | 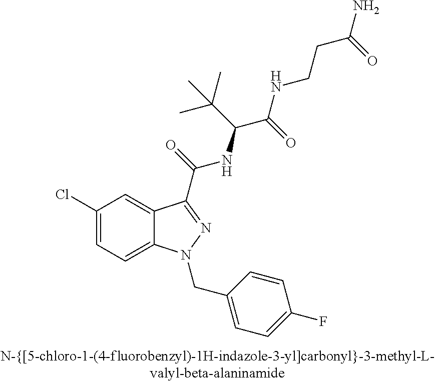 | ¹H NMR (400 MHz, DMSO-d6) δ ppm 0.94 (s, 9H) 2.24 (t, J = 6.95 Hz, 2H) 3.14-3.25 (m, 1H) 4.46 (d, J = 9.88 Hz, 1H) 5.78 (s, 2H) 6.82 (br. s., 1H) 7.16 (t, J = 8.97 Hz, 2H) 7.25-7.34 (m, 3H) 7.49 (dd, J = 8.97, 2.01 Hz, 1H) 7.59 (d, J = 9.52 Hz, 1H) 7.86 (d, J = 8.79 Hz, 1H) 8.13 (d, J = 1.83 Hz, 1H) 8.35 (t, J = 5.49 Hz, 1H) | 488 |
| 478 | 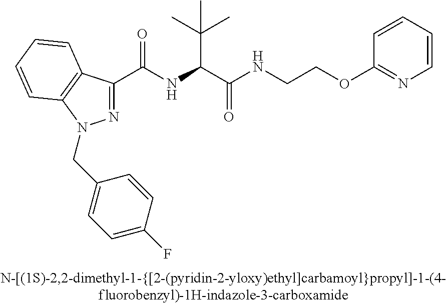 | ¹H NMR (400 MHz, CDCl3-d6) δ ppm 1.131 (s, 9H) 3.608-3.669 (m, 1H) 3.729-3.791 (m, 1H) 4.441-4.483 (q, 3H) 5.581 (s, 2H) 6.751-6.772 (d, J = 8.4 Hz, 2H) 6.876-6.907 (t, J = 6.4 Hz, 1H) 6.977-7.020 (t, J = 8.4 Hz, 2H) 7.222-7.248 (m, 3H) 7.289-7.375 (m, 2H) 7.572-7.611 (d, J = 8.4 Hz, 1H) 7.723-7.746 (d, J = 9.2 Hz, 1H) 8.123-8.133 (d, J = 4.0 Hz, 1H) 8.307-8.327 (d, J = 8.0 Hz, 1H) | 504 |
| 479 | 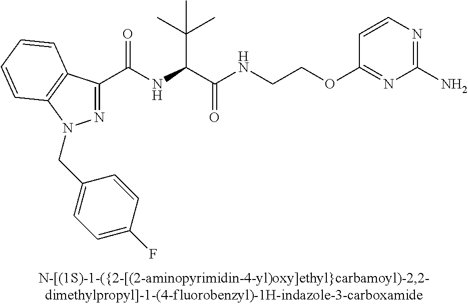 | ¹H NMR (400 MHz, CDCl3-d6) δ ppm 1.074 (s, 9H) 3.520-3.585 (m, 1H) 3.597-3.745 (m, 1H) 4.316-4.368 (m, 1H) 4.417-4.477 (m, 1H) 4.501-4.586 (m, 1H) 5.566-5.661 (d, J = 6.4 Hz, 2H) 6.040-6.056 (d, J = 6.4 Hz, 1H) 6.293-6.330 (s, 2H) 6.962-7.020 (t, J = 6.4 Hz, 2H) 7.191-7.225 (m, 3H) 7.241-7.7.281 (m, 3H) 7.299-7.375 (m, 2H) 7.707-7.771 (m, 2H) 8.246-8.267 (d, J = 8.4 Hz, 1H) | 520 |
| 480 | 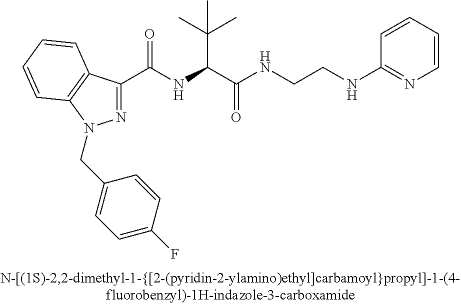 | ¹H NMR (400 MHz, CDCl3-d6) δ ppm 1.098 (s, 9H) 3.505 (m, 4H) 4.429-4.460 (d, J = 12.4 Hz, 1H) 5.583 (s, 2H) 5.745-5.775 (s, 1H) 6.493-6.554 (t, J = 12.2 Hz, 2H) 6.970-7.027 (t, J = 11.4 Hz, 2H) 7.184-7.230 (d, J-11.2 Hz, 2H) 7.260-7.290 (t, J = 6 Hz, 1H) 7.318-7.360 (t, J = 8.4 Hz, 3H) 7.439-7.472 (t, J = 6.6 Hz, 1H) 7.705-7.735 (d, J = 12 Hz, 1H) 7.975-7.992 (d, J = 6.8 Hz, 1H) 8.291-08.318 (d, J = 10.8 Hz, 1H) | 503 |
| 481 | 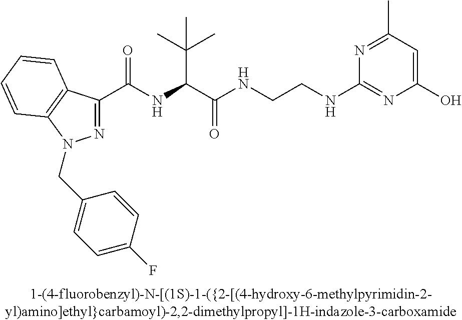 | ¹H NMR (400 MHz, CDCl3-d6) δ ppm 1.112 (s, 9H) 2.192 (s, 3H) 3.532 (s, 4H) 4.478-4.501 (d, J = 9.2 Hz, 1H) 5.504 (s, 2H) 5.561 (s, 1H) 6.941-6.963 (t, J = 4.4 Hz, 2H) 7.108-7.142 (q, 2H) 7.170-7.184 (d, J = 5.6 Hz, 1H) 7.234-7.306 (m, 3H) 7.715-7.737 (d, J = 8.8 Hz, 1H) 8.107-8.112 (d, J = 2 Hz, 1H) 8.221 (s, 1H) | 534 |
| 482 | 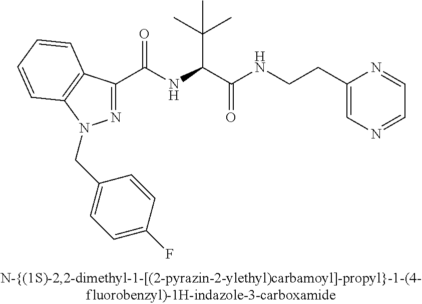 | ¹H NMR (400 MHz, CDCl3-d6) δ ppm 1.067 (s, 9H) 3.041-3.083 (t, J = 8.4 Hz, 2H) 3.658-3.801 (m, 2H) 4.471-4.503 (d, J = 12.8 Hz, 1H) 5.579 (s, 2H) 6.962-7.019 (t, J = 11.4 Hz, 3H) 7.180-7.227 (q, 2H) 7.260-7.293 (t, J = 6.6 Hz, 1H) 7.319-7.383 (q, 2H) 7.700-7.731 (m, 1H) 8.282-8.309 (d, J = 10.4 Hz, 1H) 8.382 (s, 1H) 8.434-8.464 (d, J = 12 Hz, 2H) | 489 |
| 483 | 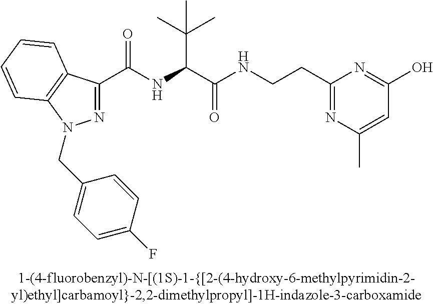 | ¹H NMR (400 MHz, CDCl3-d6) δ ppm 1.116 (s, 9H) 1.243 (s, 3H) 2.898-2.964 (s, 2H) 3.345 (s, 1H) 4.316-4.328 (s, 1H) 4.954-4.978 (d, J = 9.6 Hz, 1H) 5.445 (s, 1H) 5.550-5.640 (q, 2H) 7.023-7.035 (t, J = 4.4 Hz, 2H) 7.308-7.343 (t, J = 7 Hz, 1H) 7.365-7.434 (m, 4H) 7.936-7.956 (d, J = 8 Hz, 2H) 8.201 (s, 1H) | 519 |
| 484 | 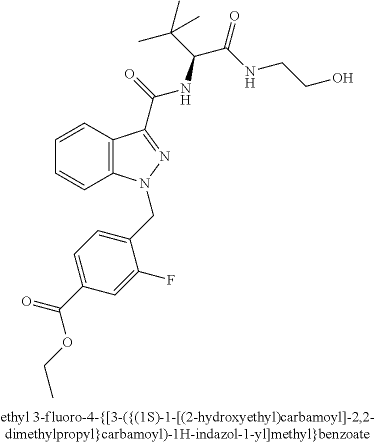 | ¹H NMR (400 MHz, DMSO-d6) δ ppm 0.90-0.98 (m, 9H) 1.27 (t, J = 7.14 Hz, 3H) 3.11 (d, J = 5.86 Hz, 1H) 3.17 (d, J = 5.86 Hz, 1H) 3.40 (q, J = 5.61 Hz, 2H) 4.28 (q, J = 7.20 Hz, 2H) 4.48 (d, J = 9.52 Hz, 1H) 4.66 (t, J = 5.31 Hz, 1H) 5.93 (s, 2H) 7.17 (t, J = 7.87 Hz, 1H) 7.30 (t, J = 7.50 Hz, 1H) 7.47 (t, J = 7.69 Hz, 1H) 7.56 (d, J = 9.88 Hz, 1H) 7.68-7.78 (m, 3H) 8.18 (d, J = 8.05 Hz, 1H) 8.30 (t, J = 5.49 Hz, 1H) | 499 |
| 485 | 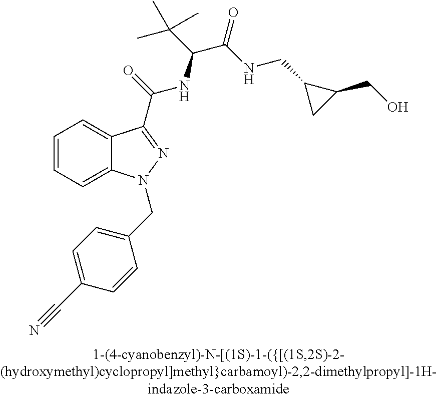 | ¹H NMR (400 MHz, DMSO-d6) δ ppm 8.34 (t, J = 5.31 Hz, 1H) 8.18 (d, J = 8.42 Hz, 1H) 7.74-7.83 (m, 3H) 7.59 (d, J = 9.52 Hz, 1H) 7.46 (t, J = 7.32 Hz, 1H) 7.27-7.37 (m, 3H) 5.91 (s, 2H) 4.48 (d, J = 9.52 Hz, 1H) 4.37-4.43 (m, 1H) 3.23-3.30 (m, 1H) 2.99-3.22 (m, 2H) 2.79-2.96 (m, 1H) 0.97 (s, 9H) 0.72-0.85 (m, 2H) 0.27-0.35 (m, 2H). | 474 |
| 486 | 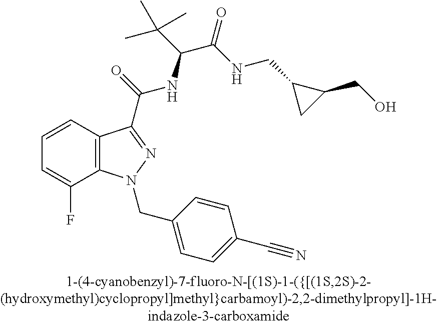 | ¹H NMR (400 MHz, DMSO-d6) δ ppm 8.34 (t, J = 5.49 Hz, 1H) 8.01 (d, J = 7.32 Hz, 1H) 7.80 (d, J = 8.42 Hz, 2H) 7.63 (d, J = 9.52 Hz, 1H) 7.22-7.33 (m, 4H) 5.93 (s, 2H) 4.48 (d, J = 9.52 Hz, 1H) 4.40 (td, J = 5.49, 3.29 Hz, 1H) 3.27 (dq, J = 10.52, 5.40 Hz, 1H) 3.00-3.21 (m, 2H) 2.80-2.97 (m, 1H) 0.97 (s, 8H) 0.73-0.86 (m, 2H) 0.28-0.35 (m, 2H). | 492 |
| 487 | 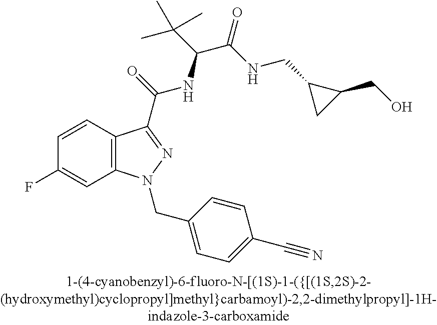 | ¹H NMR (400 MHz, DMSO-d6) δ ppm 8.33 (t, J = 5.31 Hz, 1H) 8.18 (dd, J = 8.97, 5.31 Hz, 1H) 7.80 (d, J = 8.42 Hz, 2H) 7.74 (dd, J = 9.70, 2.01 Hz, 1H) 7.57 (d, J = 9.52 Hz, 1H) 7.37 (d, J = 8.42 Hz, 2H) 7.19 (td, J = 9.06, 2.01 Hz, 1H) 5.86 (s, 2H) 4.46 (d, J = 9.88 Hz, 1H) 4.35-4.43 (m, 1H) 3.23-3.30 (m, 1H) 2.99-3.22 (m, 2H) 2.79-2.96 (m, 1H) 0.96 (s, 9H) 0.71-0.85 (m, 2H) 0.28-0.36 (m, 2H). | 492 |
| 488 | 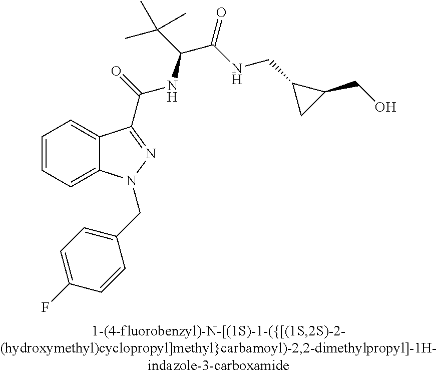 | ¹H NMR (400 MHz, DMSO-d6) δ ppm 8.34 (t, J = 5.31 Hz, 1H) 8.16 (d, J = 8.05 Hz, 1H) 7.78 (d, J = 8.79 Hz, 1H) 7.59 (d, J = 9.52 Hz, 1H) 7.45 (t, J = 7.69 Hz, 1H) 7.24-7.33 (m, 3H) 7.15 (t, J = 8.79 Hz, 2H) 5.77 (s, 2H) 4.48 (d, J = 9.52 Hz, 1H) 4.37-4.43 (m, 1H) 3.24-3.30 (m, 1H) 3.00-3.21 (m, 2H) 2.79-2.96 (m, 1H) 0.97 (s, 9H) 0.79 (ddd, J = 13.27, 6.50, 6.22 Hz, 1H) 0.29-0.35 (m, 2H). | 467 |
| 489 | 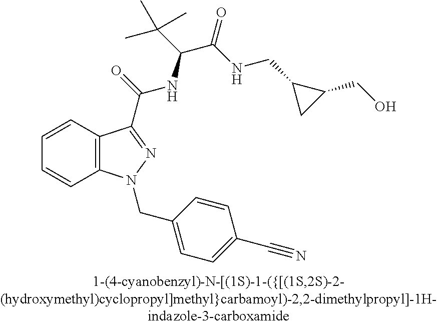 | ¹H NMR (400 MHz, DMSO-d6) δ ppm 8.32 (dt, J = 10.98, 5.49 Hz, 1H) 8.18 (d, J = 8.05 Hz, 1H) 7.73-7.82 (m, 3H) 7.60 (dd, J = 9.52, 2.20 Hz, 1H) 7.46 (t, J = 7.50 Hz, 1H) 7.27-7.37 (m, 3H) 5.92 (s, 2H) 4.53 (td, J = 5.40, 2.01 Hz, 1H) 4.48 (dd, J = 9.70, 3.48 Hz, 1H) 3.50 (dq, J = 11.67, 5.75 Hz, 1H) 3.18-3.27 (m, 1H) 2.97-3.16 (m, 2H) 0.91-1.06 (m, 11H) 0.60 (td, J = 8.33, 4.58 Hz, 1H) 0.07 (quin, J = 4.94 Hz, 1H). | 474 |
| 490 | 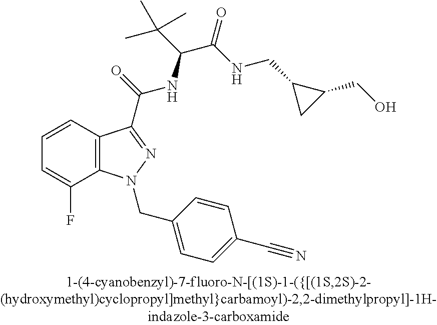 | ¹H NMR (400 MHz, DMSO-d6) δ d ppm 8.33 (ddd, J = 11.16, 5.49, 5.31 Hz, 1H) 8.01 (d, J = 7.69 Hz, 1H) 7.80 (d, J = 8.05 Hz, 2H) 7.64 (dd, J = 9.52, 1.83 Hz, 1H) 7.23-7.33 (m, 4H) 5.93 (s, 2H) 4.46-4.56 (m, 2H) 3.45-3.54 (m, J = 11.53, 5.77, 5.77, 5.49 Hz, 1H) 3.17-3.28 (m, 1H) 2.96-3.17 (m, 1H) 0.91-1.06 (m, 10H) 0.60 (td, J = 8.33, 4.58 Hz, 1H) 0.07 (dq, J = 5.12, 4.88 Hz, 1H). | 492 |
| 491 | 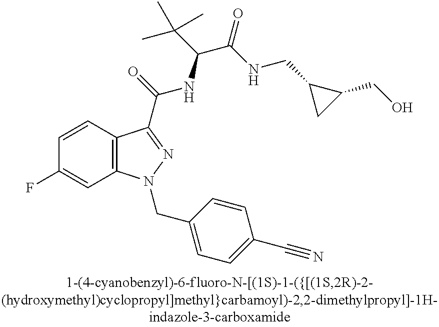 | ¹H NMR (400 MHz, DMSO-d6) δ ppm 8.32 (dt, J = 10.71, 5.45 Hz, 1H) 8.18 (dd, J = 8.97, 5.31 Hz, 1H) 7.80 (d, J = 8.42 Hz, 2H) 7.74 (dd, J = 9.70, 2.01 Hz, 1H) 7.58 (dd, J = 9.52, 2.20 Hz, 1H) 7.37 (d, J = 8.42 Hz, 2H) 7.20 (td, J = 9.15, 1.83 Hz, 1H) 5.87 (s, 2H) 4.52 (td, J = 5.31, 1.83 Hz, 1H) 4.46 (dd, J = 9.70, 3.48 Hz, 1H) 3.49 (dq, J = 11.39, 5.72 Hz, 1H) 3.16-3.27 (m, 1H) 2.96-3.15 (m, 1H) 0.86-1.06 (m, 11H) 0.59 (td, J = 8.33, 4.58 Hz, 1H) 0.07 (quin, J = 4.85 Hz, 1H). | 492 |
| 492 | 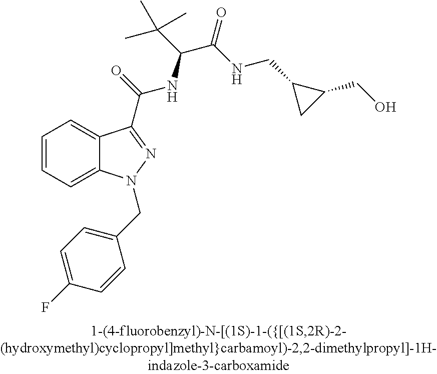 | ¹H NMR (400 MHz, DMSO-d6) δ ppm 8.32 (ddd, J = 10.80, 5.67, 5.49 Hz, 1H) 8.16 (d, J = 8.42 Hz, 1H) 7.78 (d, J = 8.42 Hz, 1H) 7.60 (dd, J = 9.70, 2.38 Hz, 1H) 7.45 (t, J = 7.50 Hz, 1H) 7.25-7.34 (m, 3H) 7.15 (t, J = 8.79 Hz, 2H) 5.77 (s, 2H) 4.53 (td, J = 5.31, 2.20 Hz, 1H) 4.48 (dd, J = 9.70, 3.48 Hz, 1H) 3.50 (dq, J = 11.44, 5.83 Hz, 1H) 3.19-3.28 (m, 1H) 2.97-3.18 (m, 1H) 0.89-1.08 (m, 11H) 0.60 (td, J = 8.24, 4.39 Hz, 1H) 0.07 (quin, J = 4.85 Hz, 1H). | 467 |
| 493 | 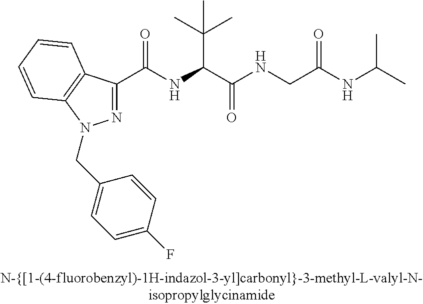 | ¹H NMR (400 MHz, CDCl3-d6) δ ppm 1.027-1.043 (d, J = 6.4 Hz, 2H), 1.084 (s, 9H), 1.113-1.129 (d, J = 6.4 Hz, 2H), 3.866-4.016 (m, 3H), 4.252-4.271 (d, J = 7.6 Hz, 1H), 5.524 (s, 2H), 6.300-6.361 (br, 1H), 6.587 (s, 1H), 6.920-6.963 (t, J = 8.6 Hz, 2H), 7.114-7.148 (m, 2H), 7.291-7.326 (m, 3H), 7.549-7.568 (br, 1H), 8.241-8.261 (d, J = 8.0 Hz, 1H) | 482 |
| 494 | 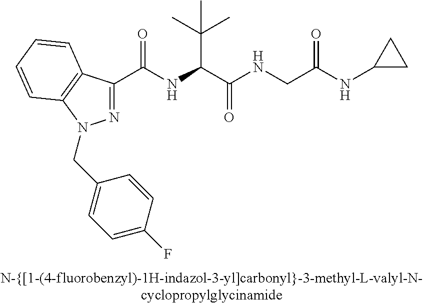 | ¹H NMR (400 MHz, CDCl3-d6) δ ppm 0.513 (s, 2H), 0.671-0.678 (d, J = 2.8 Hz, 2H), 1.083 (s, 9H), 2.665-2.675 (m, 1H), 3.750-4.050 (m, 2H), 4.159-4.177 (d, J = 6.4 Hz, 1H), 5.528 (s, 2H), 6.450 (br, 1H), 6.723 (s, 1H), 6.925-6.967 (t, J = 8.4 Hz, 2H), 7.116-7.150 (m, 2H), 7.238-7.316 (m, 3H), 7.516-7.533 (br, 1H), 8.249-8.270 (d, J = 8.4 Hz, 1H | 480 |
| 495 | 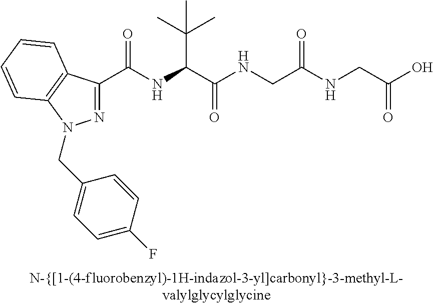 | ¹H NMR (400 MHz, CDCl3-d6) δ ppm 1.130 (s, 9H), 3.906-3.932 (d, J = 10.4 Hz, 2H), 4.058 (m, 2H), 4.585-4.605 (m, 2H), 5.577 (s, 2H), 6.979-7.021 (m, 2H), 7.180-7.212 (m, 2H), 7.293-7.377 (m, 3H), 7.642 (s, 1H), 7.793-7.814 (d, J = 8.4 Hz, 1H), 8.201-8.221 (d, J = 8 Hz, 1H) | 498 |
| 496 | 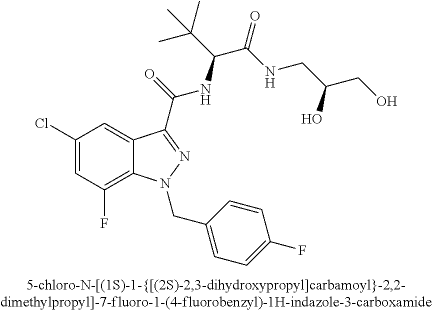 | ¹H NMR (400 MHz, DMSO-d6) δ ppm 8.23-8.30 (m, 1H), 7.97-8.01 (m, 1H), 7.63-7.69 (m, 1H), 7.51-7.56 (m, 1H), 7.20-7.27 (m, 2H), 7.12-7.20 (m, 2H), 5.79 (s, 2H), 4.72 (d, J = 5.12 Hz, 1H), 4.52-4.57 (m, 2H), 3.46-3.55 (m, 1H), 3.22-3.30 (m, 3H), 2.90-3.00 (m, 1H), 0.96 (s, 9H) | 509 |
| 497 | 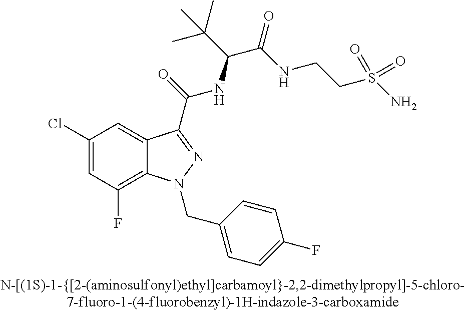 | ¹H NMR (400 MHz, DMSO-d6) δ ppm 8.50-8.56 (m, 1H), 7.97-7.99 (m, 1H), 7.61-7.67 (m, 1H), 7.51-7.57 (m, 1H), 7.21-7.28 (m, 2H), 7.13-7.20 (m, 2H), 6.91 (s, 2H), 5.79 (s, 2H), 4.44 (d, J = 9.52 Hz, 1H), 3.37-3.57 (m, 2H), 3.04-3.20 (m, 2H), 0.96 (s, 9H) | 542 |
| 498 | 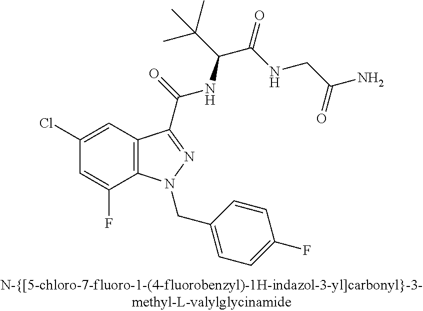 | ¹H NMR (400 MHz, DMSO-d6) δ ppm 8.46-8.53 (m, 1H), 7.97-8.00 (m, 1H), 7.65-7.70 (m, 1H), 7.51-7.56 (m, 1H), 7.33 (br. s., 1H), 7.21-7.27 (m, 2H), 7.13-7.19 (m, 2H), 7.01 (br. s., 1H), 5.79 (s, 2H), 4.53 (d, J = 9.15 Hz, 1H), 3.62-3.75 (m, 2H), 0.98 (s, 9H) | 492 |
| 499 | 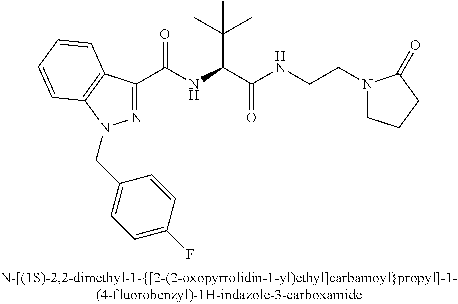 | ¹H NMR (400 MHz, CDCl3-d6) δ ppm 1.104 (s, 9H) 1.981-1.997 (m, 2H) 2.333-2.448 (m, 2H) 3.465 (m, 6H) 4.430-4.453 (d, J = 9.2 Hz, 1H) 5.587 (s, 2H) 6.731 (s, 1H) 6.984-7.021 (t, J = 7.4 Hz, 2H) 7.207-7.219 (m, 2H) 7.299-7.367 (m, 2H) 7.679-7.702 (d, J = 9.2 Hz, 1H) 8.334-8.353 (d, J = 7.6 Hz, 1H) | 494 |
| 500 | 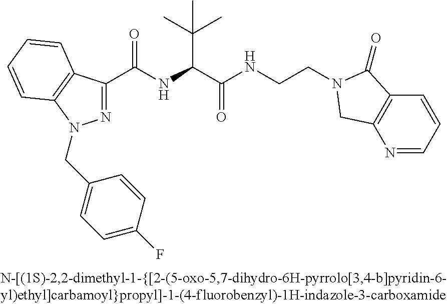 | ¹H NMR (400 MHz, CDCl3-d6) δ ppm 1.041 (s, 9H) 3.612-3.382 (m, 4H) 4.455-4.477 (d, J = 8.8 Hz, 1H) 4.823-4.974 (q, 2H) 5.577 (s, 2H) 6.990-7.027 (m, 2H), 7.120 (s, 1H) 7.215-7.228 (m, 2H) 7.310-7.417 (m, 3H) 7.570-7.591 (d, J = 8.4 Hz, 1H) 8.193-8.243 (t, J1 = 12 Hz, J2 = 8 Hz, 2H) 8.651 (s, 1H) | 543 |
| 501 | 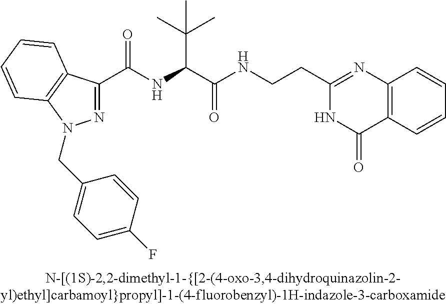 | ¹H NMR (400 MHz, CDCl3-d6) δ ppm 1.085 (s, 9H) 3.186 (s, 2H) 3.649 (s, 1H) 4.336 (s, 1H) 4.816-4.838 (d, J = 8.8 Hz, 1H) 5.342-5.466 (q, 2H) 6.989-7.032 (t, J = 8.6 Hz, 2H) 7.260-7.293 (m, 1H) 7.097-7.197 (m, 4H) 7.279-7.391 (m, 4H) 7.765-7.789 (d, J = 9.6 Hz, 1H) 7.915-7.950 (t, J = 7.0 Hz, 2H) 8.098 (s, 1H) | 555 |
| 502 | 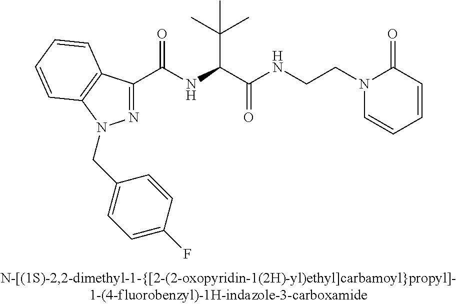 | ¹H NMR (400 MHz, CDCl3-d6) δ ppm 1.071 (s, 9H) 3.607-3.646 (m, 2H) 4.088-4.127 (m, 2H) 4.438-4.469 (d, J = 12.4 Hz, 1H) 5.574 (s, 2H) 5.994-6.039 (t, J = 9.0 Hz, 1H) 6.475-6.506 (d, J = 12.4 Hz, 1H) 6.983-7.017 (d, J = 13.6 Hz, 2H) 7.176-7.259 (m, 4H) 7.2918-7.363 (m, 2H) 7.633-7.664 (d, J = 12.4 Hz, 1H) 8.308-8.335 (d, J = 10.8 Hz, 1H) | 504 |
| 503 | 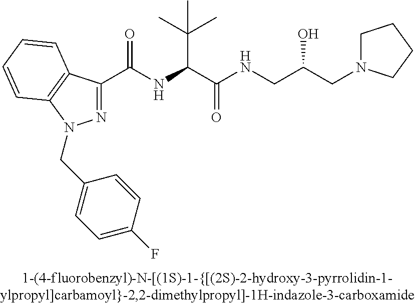 | ¹H NMR (400 MHz, CDCl3 + D2O) δ ppm 1.124 (s, 9H), 1.767 (s, 4H), 2.380-2.417 (q, 1H), 2.516-2.531 (d, J = 6 Hz, 2H), 2.631-2.684 (t, J = 10.6 Hz, 3H), 3.188-3.238 (q, 1H), 3.517-3.560 (t, J = 8.6 Hz, 1H), 3.830-3.840 (d, J = 4 Hz, 1H), 4.445-4.462 (d, J = 6.8 Hz, 2H), 5.583 (s, 2H), 6.979-7.022 (t, J = 8.6 Hz, 2H), 7.188-7.241 (q, 2H), 7.278-7.379 (m, 2H), 7.699-7.723 (d, J = 9.6 Hz, 1H), 8.326-8.346 (d, J = 8 Hz, 1H) | 510 |
| 504 | 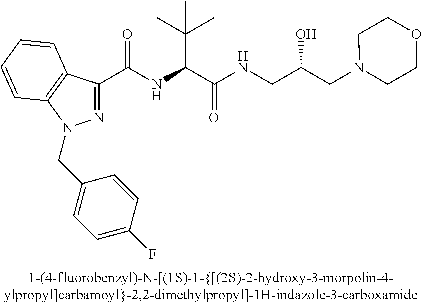 | ¹H NMR (400 MHz, CDCl3-d6) δ ppm 1.130 (s, 9H), 2.369-2.428 (m, 4H), 2.598 (s, 2H), 3.224-3.273 (q, 1H), 3.501-3.557 (m, 1H), 3.637-3.854 (m, 4H), 3.863 (s, 1H), 4.412-4.435 (d, J = 9.2 Hz, 1H), 5.585 (s, 1H), 6.376 (s, 1H), 6.984-7.027 (t, J = 8.6 Hz, 2H), 7.189-7.242 (m, 2H), 7.279-7.242 (m, 2H), 7.279-7.384 (m, 2H), 7.670-7.693 (d, J = 9.2 Hz, 1H), 8.312-8.331 (d, J = 7.6 Hz, 1H) | 526 |
| 505 | 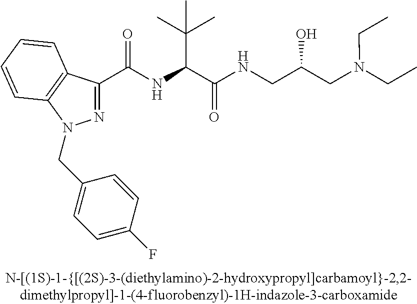 | ¹H NMR (400 MHz, CDCl3-d6) δ ppm 0.96-0.995 (t, J = 7 Hz, 6H), 1.127 (s, 9H), 2.309-2.533 (m, 4H), 2.568-2.637 (m, 2H), 3.170-3.219 (q, 1H), 3.507-3.564 (m, 1H), 3.720-3.760 (q, 1H), 4.435-4.458 (d, J = 9.2 Hz, 1H), 5.583 (s, 2H), 6.379-6.404 (t, J = 5 Hz, 1H), 6.979-7.021 (t, J = 8.4 Hz, 2H), 7.187-7.379 (m, 2H), 7.187-7.379 (m, 4H), 7.689-7.713 (d, J = 9.6 Hz, 1H), 8.329-8.349 (d, J = 8 Hz, 1H) | 512 |
| 506 | 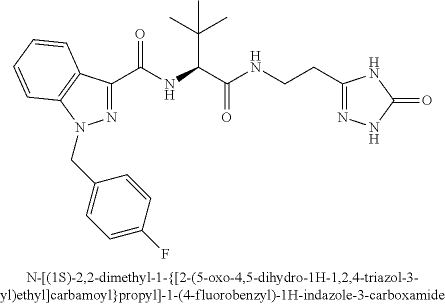 | ¹H NMR (400 MHz, CDCl3-d6) δ ppm 1.125 (s, 9H) 2.690-2.726 (d, J = 14.4 Hz , 1H) 2.938-2.966 (t, J = 11.2 Hz, 1H) 3.415 (s, 1H) 4.048 (s, 1H) 4.572-4.598 (d, J = 10.4 Hz, 1H) 5.493 (s, 2H) 6.984-7.026 (t, J = 8.4 Hz, 2H) 7.071 (s, 1H) 7.163-7.240 (m, 4H) 7.774-7.800 (d, J = 10.4 Hz, 1H) 8.163-8.183 (d, J = 8.0 Hz, 1H) 8.524 (s, 1H) | 494 |
| 507 | 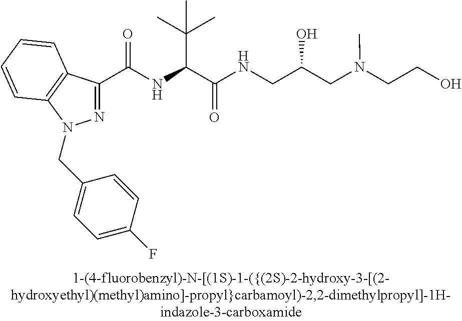 | ¹H NMR (400 MHz, CDCl3-d6) δ ppm 1.133 (s, 9H) 2.348 (s, 2H) 2.459-2.646 (m, 4H) 3.361 (m, 1H) 3.472-3.506 (m, 1H) 3.629-3.676 (m, 2H) 3.885-3.897 (d, J = 4.8 Hz, 1H) 4.411-4.433 (d, J = 8.8 Hz, 1H) 5.589 (s, 2H) 6.702 (s, 1H), 6.988-7.029 (t, J = 8.2 Hz, 2H) 7.192-7.226 (m, 2H) 7.288-7.368 (m, 2H) 7.681-7.704 (d, J = 9.2 Hz, 1H) 8.315-8.334 (d, J = 7.6 Hz, 1H) | 514 |
| 508 | 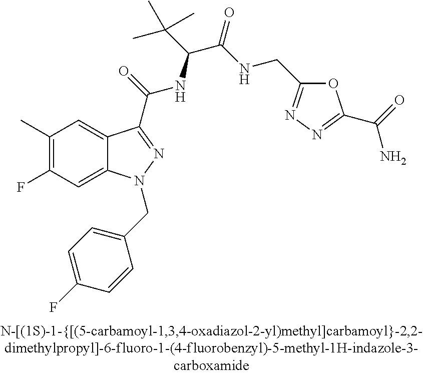 | ¹H NMR (400 MHz, DMSO-d6) δ ppm 9.15 (t, J = 5.49 Hz, 1H) 8.58 (s, 1H) 8.19 (s, 1H) 8.02 (d, J = 7.32 Hz, 1H) 7.70 (d, J = 10.25 Hz, 1H) 7.57 (d, J = 9.52 Hz, 1H) 7.31 (dd, J = 8.79, 5.49 Hz, 2H) 7.15 (t, J = 8.79 Hz, 2H) 5.70 (s, 2H) 4.49-4.73 (m, 3H) 2.32 (s, 3H) 0.97 (s, 9H). | 540 |
| 509 | 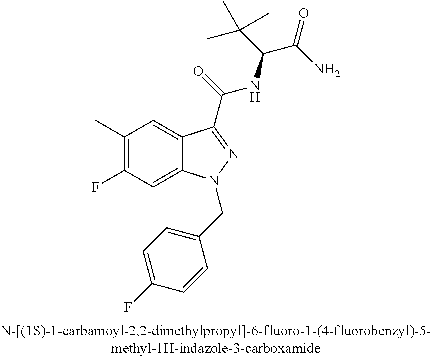 | ¹H NMR (400 MHz, DMSO-d6) δ ppm 8.03 (d, J = 7.32 Hz, 1H) 7.67-7.77 (m, 2H) 7.56 (d, J = 9.52 Hz, 1H) 7.26-7.34 (m, 2H) 7.15 (t, J = 8.79 Hz, 2H) 5.70 (s, 2H) 4.43 (d, J = 9.52 Hz, 1H) 3.73 (s, 1H) 2.32 (s, 3H) 0.96 (s, 9H).). | 415 |
| 510 | 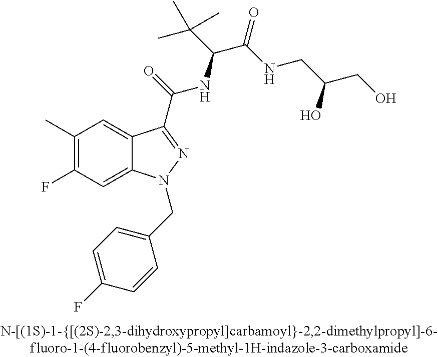 | ¹H NMR (400 MHz, DMSO-d6) δ ppm 8.26 (t, J = 5.49 Hz, 1H) 8.03 (d, J = 7.32 Hz, 1H) 7.70 (d, J = 10.25 Hz, 1H) 7.59 (d, J = 9.52 Hz, 1H) 7.31 (dd, J = 8.79, 5.49 Hz, 2H) 7.15 (t, J = 8.79 Hz, 2H) 5.70 (s, 2H) 4.71 (br. s., 1H) 4.49-4.59 (m, 2H) 3.47-3.54 (m, 1H) 3.22-3.30 (m, 3H) 2.90-2.99 (m, 1H) 2.32 (s, 3H) 0.95 (s, 9H). | 489 |
| 511 | 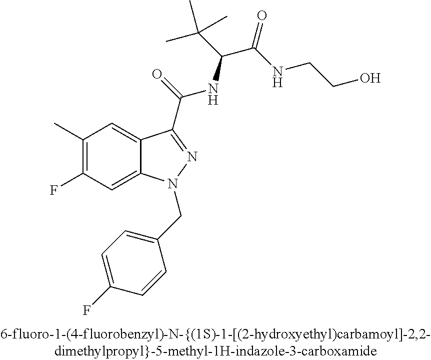 | ¹H NMR (400 MHz, DMSO-d6) δ ppm 8.30 (t, J = 5.49 Hz, 1H) 8.03 (d, J = 7.32 Hz, 1H) 7.70 (d, J = 10.25 Hz, 1H) 7.57 (d, J = 9.88 Hz, 1H) 7.31 (dd, J = 8.60, 5.67 Hz, 2H) 7.15 (t, J = 8.97 Hz, 2H) 5.70 (s, 2H) 4.66 (br. s., 1H) 4.47 (d, J = 9.52 Hz, 1H) 3.40 (t, J = 5.67 Hz, 2H) 3.05-3.25 (m, 2H) 2.31 (s, 3H) 0.95 (s, 9H). | 459 |
| 512 | 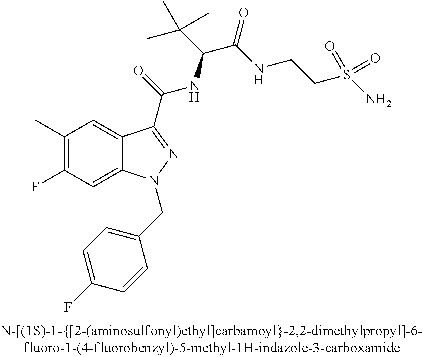 | ¹H NMR (400 MHz, DMSO-d6) δ ppm 8.51 (t, J = 5.49 Hz, 1H) 8.02 (d, J = 7.69 Hz, 1H) 7.71 (d, J = 10.25 Hz, 1H) 7.56 (d, J = 9.52 Hz, 1H) 7.32 (dd, J = 8.42, 5.49 Hz, 2H) 7.16 (t, J = 8.79 Hz, 2H) 6.91 (s, 2H) 5.70 (s, 2H) 4.42 (d, J = 9.52 Hz, 1H) 3.36-3.58 (m, 2H) 3.03-3.20 (m, 2H) 2.32 (s, 3H) 0.95 (s, 9H). | 522 |
| 513 | 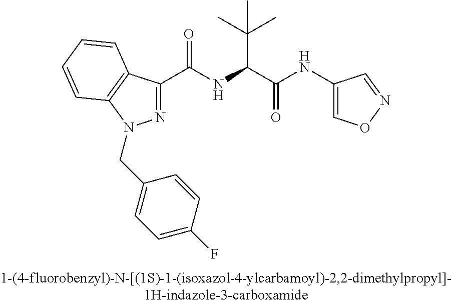 | ¹H NMR (400 MHz, CDCl3-d6) δ ppm 1.220 (s, 9H) 4.867 (d, J = 9.6 Hz, 1H) 5.620 (s, 1H) 7.023 (t, J = 8.6 Hz, 3H) 7.208~7.258 (m, 2H) 7.282~7.396 (m, 2H) 7.809~7.833 (m, 1H) 8.179 (s, 1H) 8.236 (s, 1H) 8.923 (s, 1H) 9.187 (s, 1H) | 450 |
| 514 | 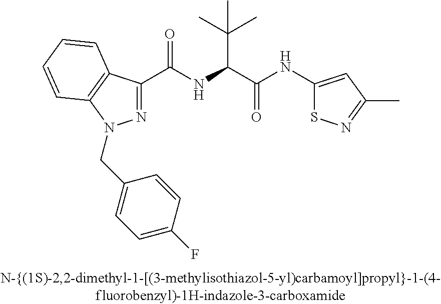 | ¹H NMR (400 MHz, CDCl3-d6) δ ppm 1.237 (s, 9H) 2.166 (s, 3H) 4.986 (d, J = 9.6 Hz, 1H) 5.628 (s, 2H) 6.321 (s, 1H) 7.002~7.045 (m, 2H) 7.225~7.255 (m, 2H) 7.271~7.275 (m, 1H) 7.385~7.402 (m, 2H) 7.881 (d, J = 9.2 Hz, 1H) 8.253 (d, J = 9.2 Hz, 1H) | 480 |
| 515 | 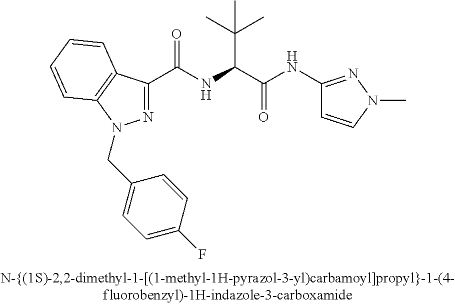 | ¹H NMR (400 MHz, CDCl3-d6) δ ppm 1.157 (s, 9H) 3.808 (s, 9H) 4.598 (d, J = 9.2 Hz, 1H) 5.591 (s, 1H) 6.671 (d, J = 3.2 Hz, 1H) 6.976~7.033 (m, 2H) 7.183~7.243 (m, 3H) 7.289~7.370 (m, 3H) 7.747 (d, J = 12 Hz 1H) 8.341~7.368 (m, 2H) | 463 |
| 516 | 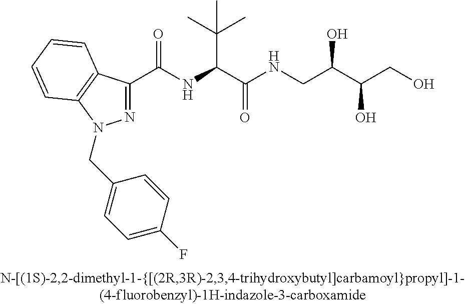 | ¹H NMR (400 MHz, CD3OD-d6) δ ppm1.098 (s, 9H), 3.302-3.318 (m, 1H), 3.544-3.562 (m, 1H), 3.588-3.617 (m, 4H), 3.735 (m, 1H), 4.521-4.544 (d, 1H , J = 9 Hz), 5.715 (s, 2H), 7.029-7.073 (t, 2H, J = 8.8 Hz), 7.286-7.295 (m, 3H), 7.308-7.331 (m, 1H), 7.436-7.439 (d, 1H, J = 1.2 Hz), 7.589-7.610 (d, 1H, J = 8.4 Hz), 8.210-8.233 (dd, 1H, J1 = 8.2 Hz, J2 = 0.4 Hz), 8.360 (s, 1H) | 487 |
| 517 | 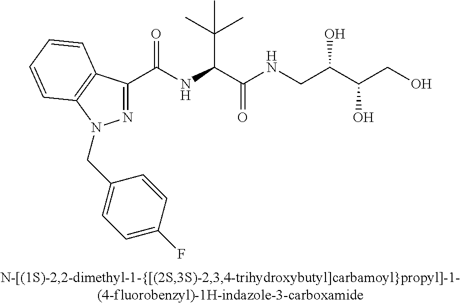 | ¹H NMR (400 MHz, CD3OD-d6) δ ppm 1.102 (s, 9H), 3.458-3.504 (dd, 1H, J1 = 4.8 Hz, J2 = 13.8 Hz), 3.560-3.625 (m, 3H), 3.741-3.748 (m, 3H), 4.529 (s, 1H), 5.712 (s, 2H), 7.027-7.070 (m, 2H), 7.267-7.328 (m, 3H), 7.412-7.453 (m, 1H), 7.583-7.605 (d, 1H, J = 8.8 Hz), 8.211-8.232 (d, 1H, J = 8.4 Hz) | 487 |
| 518 | 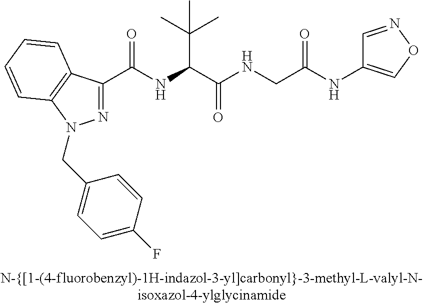 | ¹H NMR (400 MHz, CDCl3-d6) δ ppm 1.197 (s, 9H) 4.021 (d, J = 5.2 Hz, 1H), δ 4.105 (d, J = 5.6 Hz, 2H) 4.272~4.334 (m, 1H) 5.603 (s, 2H) 6.679 (m, 1H) 6.996~7.039 (m, 2H) 7.179~7.213 (m, 2H) 7.292~7.407 (m, 3H) 7.611 (d, = 5.2 Hz, 1H) 8.1911 (d, = 8.4 Hz 1H) 8.613 (s, 1H) 9.005 (s, 1H) 9.327 (s, 1H) | 507 |
| 519 | 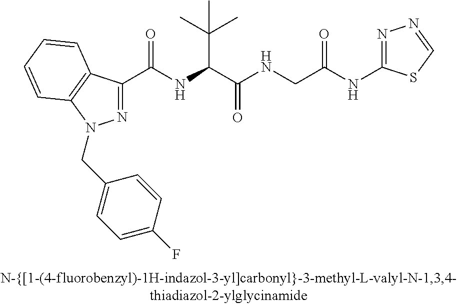 | ¹H NMR (400 MHz, CDCl3-d6) δ ppm 1.084 (s, 9H), δ 4.142 (d, J = 5.6 Hz, 1H) 4.422 (d, J = 6 Hz, 1H) 4.557 (d, J = 8 Hz, 1H) 5.523 (d, J = 4.4 Hz, 2H) 6.958~7.001 (m, 2H) 7.139~7.174 (m, 2H) 7.209~7.317 (m, 3H) 7.656 (d, J = 8.4 Hz, 1H) 8.058~8.086 (m, 1H) 8.396 (d, J = 8 Hz, 1H) 8.816 (s, 1H) | 524 |
| 520 | 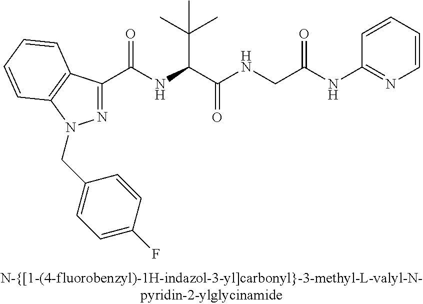 | ¹H NMR (400 MHz, CDCl3-d6) δ ppm 1.158 (s, 9H) 4.179~4.206 (m, 2H) 4.677~4.700 (d, J = 9.2 Hz, 1H) 5.540 (s, 2H) 6.954~6.997 (m, 3H) 7.147~7.356 (m, 5H) 7.545~7.652 (m, 2H) 7.769 (m, 1H) 7.791 (m, 1H) 8.075 (m, 1H) 8.216 (s, 1H) | 517 |
| 521 | 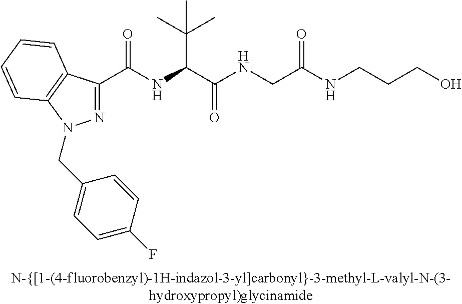 | ¹H NMR (400 MHz, CDCl3) δ ppm 1.160 (s, 9H), 1.657-1.679 (m, 2H) 3.404-3.439 (m, 2H) 3.578-3.606 (m, 2H) 3.819-3.874 (dd, J1 = 16.8 Hz, J2 = 5.2 Hz, 1H) 4.111-4.170 (dd, J1 = 16.8 Hz, J2 = 6.8 Hz, 1H) 4.242-4.260 (d, J = 7.2 Hz, 1H) 5.598 (s, 2H) 6.672 (s, 1H), 6.994-7.037 (m, 2H) 7.184-7.219 (m, 2H) 7.287-7.388 (m, 3H) 7.612-7.630 (d, J = 7.2 Hz, 1H), 8.243-8.263 (d, J = 8.0 Hz, 1H) | 498 |
| 522 | 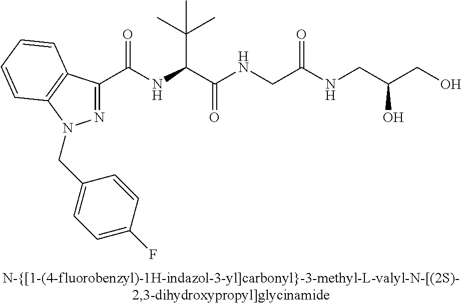 | ¹H NMR (400 MHz, CDCl3-d6) δ ppm 1.137 (s, 9H), 3.286 (br, 1H), 3.435-3.506 (m, 4H), 3.759-3.849 (m, 2H), 4.110-4.189 (m, 1H), 4.335-4.353 (d, J = 7.2 Hz, 1H), 5.572 (s, 2H), 6.972-7.015 (m, 2H), 7.162-7.197 (dd, J1 = 8.8 Hz, J2 = 5.6 Hz, 2H), 7.278-7.378 (m, 3H), 7.441 (s, 1H), 7.664-7.682 (d, J = 7.2 Hz, 1H), 8.218-8.238 (d, J = 8.0 Hz, 1H) | 514 |
| 523 | 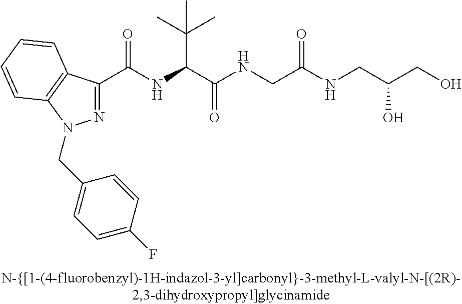 | ¹H NMR (400 MHz, CDCl3-d6) δ ppm 1.156 (s, 9H) 3.363-3.527 (m, 5H) 3.775-3.836 (m, 2H) 4.178-4.265 (m, 2H) 5.590 (s, 2H) 6.988-7.031 (m, 2H), 7.151-7.210 (m, 3H) 7.282-7.374 (m, 3H) 7.445 (s, 1H) 7.646-7.663 (d, J = 6.8 Hz, 2H) 8.231-8.252 (d, J = 8.4 Hz, 1H) | 514 |

1. A compound according to Formula I: 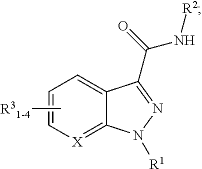
or a pharmaceutically acceptable salt thereof, wherein
X is CH or N;
R¹ is
R⁴₁₋₅-aryl-(CH₂)ₙ— or
R⁵₁₋₅-heteroaryl-(CH₂)ₙ—; wherein
each R⁴ is independently H, halo, cyano, NH₂—C(O)—, C₁-C₆ trifluoromethyl or C₁-C₆ alkoxy-C(O)—;
each R⁵ is independently H or C₁-C₆ alkyl;
R² is
NR¹¹R¹²—C(O)—R¹³CH—,
R¹⁴—C(O)—NR¹⁵—(CH₂)ₙ—R¹³CH—,
C₁-C₆ alkoxy-C(O)—(CH₂)—NR¹⁵—C(O)—R¹³CH—,
NR¹⁷R¹⁸—C(O)—(CH₂)ₙ—NR¹⁹—C(O)—R¹³CH—,
R²⁰—SO₂—NR²¹—(CH₂)ₙ—R¹³CH—,
R²²R²³CH—,
R²⁴₁₋₅-heteroaryl,
R²⁴₁₋₅-heteroaryl-R¹³CH—,
R²⁴₁₋₅-heteroaryl-NR¹⁵—C(O)—R¹³CH—,
R²⁵₁₋₅-heterocyclyl,
R²⁵₁₋₅-heterocyclyl-(CH₂)ₙ—,
R²⁶₁₋₅—C₃-C₇ cycloalkyl,
NR²⁷R²⁸—(CH₂)ₙ—NR²⁹—C(O)—R¹³CH—,
R³⁰—SO₂—NR³¹—(CH₂)ₙ—NR¹⁵—C(O)—R¹³CH—,
R³⁰—SO₂—(CH₂)ₙ—NR³¹—C(O)—R¹³CH—,
R³²—C(O)—R³³CH—NR³⁴—C(O)—R¹³CH—,
R³²—C(O)—(CH₂)ₙ—NR³⁴—C(O)—R¹³CH—,
R³⁵₁₋₅-heteroaryl-(CH₂)ₙ—NR³⁶—C(O)₇R¹³CH—,
R³⁷₁₋₅-heterocyclyl-(CH₂)ₙ—NR³⁶—C(O)—R¹³CH—,
R³⁷₁₋₅-heterocyclyl-C(O)—R¹³CH—,
R³⁸₁₅-aryl-R³⁹C—NR⁴⁰—C(O)—R¹³CH—,
R³⁸₁₋₅-aryl-(CH₂)ₙ—NR⁴⁰—C(O)—R¹³CH—,
R⁴¹₁₋₅-aryl-(CH₂)ₙ—,
NR¹⁷R¹⁸—C(O)—CH(R⁴²)—NR¹⁹—C(O)—R¹³CH—, or
R⁴³—CH(OH)—CH₂—NR¹⁹—C(O)—R¹³CH—;
wherein
R¹¹ and R¹² are independently H, OH, C₁-C₆ alkyl, C₁-C₆ haloalkyl, OH—C₁-C₆ alkyl, (OH)₂—C₁-C₆ alkyl, (OH)₃—C₄-C₆ alkyl, C₁-C₆ alkoxy-(CH₂)—, C₃-C₇ cycloalkyl, benzo-fused C₃-C₇ cycloalkyl, cyano-C₁-C₆ alkyl, NH₂—C(NH)—C₁-C₆ alkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl-, OH—C₃-C₇ cycloalkyl-, C₁-C₆ alkoxy-C(O)—C₃-C₇ cycloalkyl-, (C₁-C₆ alkoxy-aryl)-C₃-C₇ cycloalkyl-, NH₂—C(O)—C₃-C₇ cycloalkyl-, OH-aryl, or R²⁴₁₋₅-heteroaryl-O—(CH₂)ₙ—;
R¹³ is H, C₁-C₆ alkyl, OH—C₁-C₆ alkyl, aryl, aryl-(CH₂)—, or C₃-C₇ cycloalkyl;
R¹⁴ is (C₁-C₆ alkyl)₂N—, aryl, C₁-C₆ alkyl, or C₃-C₇ cycloalkyl;
R¹⁵, R²¹, R²⁹, R³¹, R³⁴, and R⁴⁰ are independently H or C₁-C₆ alkyl;
R¹⁶ is OH or C₁-C₆ alkoxy;
R¹⁷ and R¹⁸ are independently H, C₁-C₆ alkyl, C₃-C₇ cycloalkyl, OH—C₁-C₆ alkyl, (OH)₂—C₁-C₆ alkyl, or R²⁴₁₋₅-heteroaryl-;
each R¹⁹ is independently H or C₁-C₆ alkyl;
R²⁰ is C₁-C₆ alkyl, C₁-C₆ haloalkyl, or (C₁-C₆ alkyl)₂N—;
R²² and R²³ are independently C₁-C₆ alkyl, C₃-C₇ cycloalkyl-(CH₂)ₙ—, OH—C₁-C₆ alkyl, aryl, or aryl-OH—C₁-C₆ alkylene;
each R²⁴ is independently H, C₁-C₆ alkyl, C₃-C₇ cycloalkyl, C₁-C₆ haloalkyl, oxo, OH, NH₂, C₁-C₆ alkoxy-C(O)—, NH₂—C(O)—(CH₂)ₙ—, NH₂—C(O)—, NH₂—C(O)—NH—, OH—C(O)—, NH₂—C(O)—(CH₂)ₙ—NH—C(O)—, (OH)₂—C₁-C₆ alkyl-NH—C(O)—, OH—C₁-C₆ alkyl-NH—C(O)—, or C₃-C₇ cycloalkyl-C(O)—NH—;
each R²⁵ is independently H or oxo;
each R²⁶ is independently H, OH, OH—C₁-C₆ alkyl, aryl-(CH₂)ₙ—O—, NH₂—C(O)— or C₁-C₆ alkoxy-C(O)—;
R²⁷ and R²⁸ independently are H, NH₂—C(O)—, C₃-C₇ cycloalkyl-C(O)—, or R²⁴₁₋₅-heteroaryl-;
R³⁰ is C₁-C₆ alkyl, C₃-C₇ cycloalkyl, NH₂, C₁-C₆ alkyl-NH—, C₃-C₇ cycloalkyl-(CH₂)ₙ—NH—, morpholin-4-yl, or R³⁸₁₋₅-phenyl;
R³² is OH or C₁-C₆ alkoxy-;
each R³³ is independently H, C₁-C₆ alkyl, or OH—C₁-C₆ alkyl;
each R³⁵ is independently H, C₁-C₆ alkyl, NH₂—C(O)—, C₁-C₆ alkoxy-C(O)—, C₃-C₇ cycloalkyl, OH, phenyl, or heteroaryl, or two adjacent R³⁵ groups may together form —(CH₂)₃₋₆—;
each R³⁶ is independently H, C₁-C₆ alkyl, C₁-C₆ alkoxy-, or NH₂—C(O)—;
each R³⁷ is independently H, NH₂C(O)—, OH, halo, cyano, oxo, OH—C₁-C₆ alkyl, (OH)₂—C₁-C₆ alkyl, NH₂C(O)—(CH₂)ₙ—, NH₂C(O)—(CH₂)ₙ—C(O)—, NH₂C(O)—NH—(CH₂)ₙ—, C₁-C₆ alkyl-NH—C(O)—O—, (OH)—C₁-C₆ alkyl-NH—C(O)—, (OH)₂—C₁-C₆ alkyl-NH—C(O)—, C₁-C₆ alkyl-C(O)—, C₁-C₆ alkoxy-C(O)—, C₃-C₇ cycloalkyl-C(O)—NH—(CH₂)ₙ—, C₁-C₆ alkyl-SO₂—, C₃-C₇ cycloalkyl-SO₂—, or C₃-C₇ cycloalkyl-SO₂—NH—(CH₂)ₙ—;
each R³⁸ is independently H, NH₂SO₂—, cyano, heteroaryl, OH, halo, C₁-C₆ alkoxy, OH—C(O)—, or C₁-C₆ alkoxy-C(O)—;
each R³⁹ is independently H, C₁-C₆ alkyl, or OH—C₁-C₆ alkyl;
each R⁴¹ is independently H, C₁-C₆ alkoxy or halo;
R⁴² is H, C₁-C₆ alkyl, OH—C₁-C₆ alkyl, aryl, aryl-(CH₂)ₙ— or NH₂—C(O)—CH₂;
R⁴³ is OH—C(O)—, C₁-C₆ alkoxy-C(O)—, NH₂—C(O)— or R⁴⁴R⁴⁵NCH₂—; and
R⁴⁴ and R⁴⁵ are independently C₁-C₆ alkyl or OH—C₁-C₆ alkyl, or
R⁴⁴ and a R⁴⁵ together with the nitrogen atom to which they are attached form a pyrrolidine, piperidine or morpholine ring;
n is an integer from 1 to 6; and
each R³ is independently H, halo, C₁-C₆ alkyl, aryl, NH₂—C(O)—, C₁-C₆ alkoxy or heteroaryl.

2. The compound of claim 1 wherein
X is CH or N;
R¹ is R⁴₁₋₅-benzyl, R⁵₁₋₅-isoxazolyl-CH₂— or R⁵₁₋₅-pyridinyl-CH₂—; wherein
each R⁴ is H, fluoro, cyano, NH₂—C(O)—;
each R⁵ is independently H or CH₃;
R² is NR¹¹R¹²—C(O)—R¹³CH—, R¹⁴C(O)—NR¹⁵— CH₂—R¹³CH—, R¹⁶—C(O)—R¹³CH—, (CH₃)₃C—O—C(O)—CH₂—NR¹⁵—C(O)—R¹³CH—, NR¹⁷R¹⁸—C(O)—CH₂—NR¹⁹—C(O)—R¹³CH—, NR¹⁷R¹⁸—C(O)—(CH₂)₂—NR¹⁹—C(O)—R¹³CH—, R²⁰—SO₂—NR²¹—CH₂—R¹³CH—,
R²²R²³CH—, R²⁴₁₋₅-dihydroimidazolyl, R²⁴₁₋₅-isoxazolyl, R²⁴₁₋₅-thiadiazolyl, R²⁴₁₋₅-isoxazolyl-R¹³CH—, R²⁴₁₋₅-oxazolyl-R¹³CH—, R²⁴₁₋₅-furyl-R¹³CH—, R²⁴₁₋₅—-oxadiazolyl-R¹³CH—, R²⁴₁₋₅-triazolyl-R¹³CH—, R²⁴₁₋₅-dihydroisoxazolyl-R¹³CH—, R²⁴₁₋₅-tetrazolyl-R¹³CH—, R²⁴₁₋₅-isoxazolyl-NR¹⁵—C(O)—R¹³CH—, R²⁴₁₋₅-thiadiazolyl-NR¹⁵—C(O)—R¹³CH—, R²⁵₁₋₅-tetrahydrofuranyl, R²⁵₁₋₅-tetrahydrofuranyl-CH₂—, R²⁶₁₋₅-cyclohexyl,
R²⁶₁₋₅-tetrahydronapthyl, R²⁶₁₋₅-dihydroindenyl, NR²⁷R²⁸—(CH₂)₂—NR²⁹—C(O)—R¹³CH—, R³⁰—SO₂—NR³¹—(CH₂)₂—NR¹⁵—C(O)—R¹³CH—, R³⁰—SO₂—(CH₂)₂—NR³¹—C(O)—R¹³CH—, R³²—C(O)—R³³CH—NR³⁴—C(O)—R¹³CH—, R³²—C(O)—(CH₂)₂—NR³⁴—C(O)—R¹³CH—, R³⁵₁₋₅-oxadiazole-(CH₂)₂—NR³⁶—C(O)—R¹³CH—, R³⁵₁₋₅-oxadiazole-C₁₋₁₂—NR³⁶—C(O)—R¹³CH—, R³⁵₁₋₅-pyridinyl-CH₂—NR³⁶—C(O)—R¹³CH—, R³⁵₁₋₅-tetrazolyl-CH₂—NR³⁶—C(O)—R¹³CH—, R³⁷₁₋₅-tetrahydropyranyl-CH₂—NR³⁶—C(O)—R¹³CH—, R³⁷₁₋₅-piperidinyl-C(O)—R¹³CH—, R³⁷₁₋₅-pyrrolidinyl-C(O)—R¹³CH—, R³⁷₁₋₅-morpholinyl-(CH₂)₂—NR³⁶—C(O)—R¹³CH—, R³⁷₁₋₅-piperidinyl-(CH₂)₂—NR³⁶—C(O)—R¹³CH—, R³⁷₁₋₅-piperazinyl-(CH₂)₂—NR³⁶—C(O)—R¹³CH—, R³⁷₁₋₅-tertrahydropyranyl-(CH₂)₂—NR³⁶—C(O)—R¹³CH—, R³⁸₁₋₅-phenyl-R³⁹C—NR⁴⁰—C(O)—R¹³CH—, R³⁸₁₋₅-phenyl-(CH₂)₂—NR⁴⁰—C(O)—R¹³CH—, R³⁸₁₋₅-phenyl-(CH₂)₃—NR⁴⁰—C(O)—R¹³CH— or
R⁴¹₁₋₅-benzyl; wherein
R¹¹ and R¹² independently are H, CH₃, (CH₃)₂CH—, cyclobutyl, cyclopropyl, CH₃—O—(CH₂)₂—, OH-ethyl, OH-propyl, (OH)₂-propyl, cyano-CH₂—, (OH—CH₂)₂—CH—, OH—cyclopropyl-CH₂—, OH-cyclopentyl-CH₂—, OH-methyl-cyclopropyl or OH-phenyl;
R¹³ is H, (CH₃)₃C, (CH₃)₂CHCH₂—, (CH₃)₂CH—, OH-ethyl, benzyl, phenyl, or cyclohexyl;
R¹⁴ is (CH₃CH₂)₂N—, phenyl, (CH₃)₃C—, or cyclopropyl;
R¹⁵, R²¹, R²⁹, R³¹, R³³, R³⁴, R³⁶, R³⁹ and R⁴⁰ are independently H or CH₃;
R¹⁶ is OH or CH₃O;
R¹⁷, R¹⁸ and R¹⁹ are independently H or CH₃;
R²⁰ is (CH₃)₂CH—, CH₃, CF₃, or (CH₃)₂N—;
R²² and R²³ are independently (CH₃)₃C—, (CH₃)₂CH—, cyclohexyl-CH₂—, OHCH₂, phenyl, OH-isopropyl, OH-ethyl, or phenyl-OHCH—;
each R²⁴ is independently H, CH₃, CH₃CH₂—, (CH₃)₃C—, cyclopropyl, CF₃, oxo, NH₂, CH₃CH₂—O—C(O)—, NH₂—C(O)—CH₂—, NH₂—C(O)—, NH₂—C(O)—NH—, OH—C(O)—, NH₂—C(O)—CH₂—NH—C(O)—, (OH)₂-propyl-NH—C(O)— or OH-ethyl-NH—C(O)—;
each R²⁵ is independently H or oxo;
each R²⁶ is independently H, OH, OHCH₂, benzyl-O—, NH₂—C(O)— or CH₃CH₂—O—C(O)—;
R²⁷ and R²⁸ are independently H, NH₂—C(O)—, or cyclopropyl-C(O)—;
R³⁰ is CH₃, cyclopropyl or NH₂;
R³² is OH;
each R³⁵ is independently H, CH₃, NH₂—C(O)—, CH₃CH₂—O—C(O)—, or cyclopropyl;
each R³⁷ is independently H, NH₂C(O)— or OH;
each R³⁸ is independently H, NH₂SO₂—, cyano, tetrazolyl, OH, chloro, CH₃—O—, or CH₃—O—C(O)—;
each R⁴¹ is independently H, CH₃O or fluoro; and
each R³ is independently H, CH₃, chloro, bromo, fluoro, phenyl, NH₂—C(O)—, CH₃O, pyridinyl or oxazolyl.

3. The compound of claim 2 wherein
X is CH or N;
R¹ is 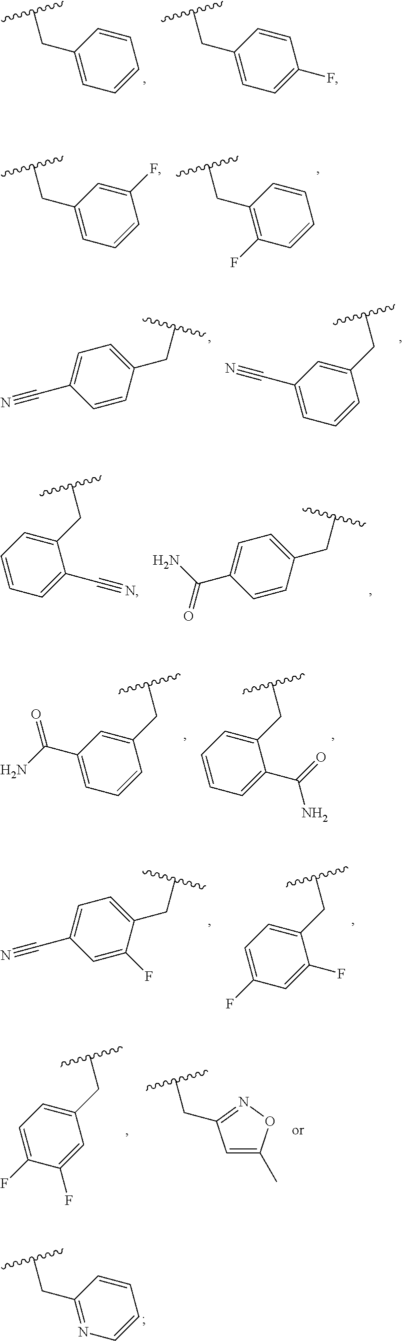
R² is 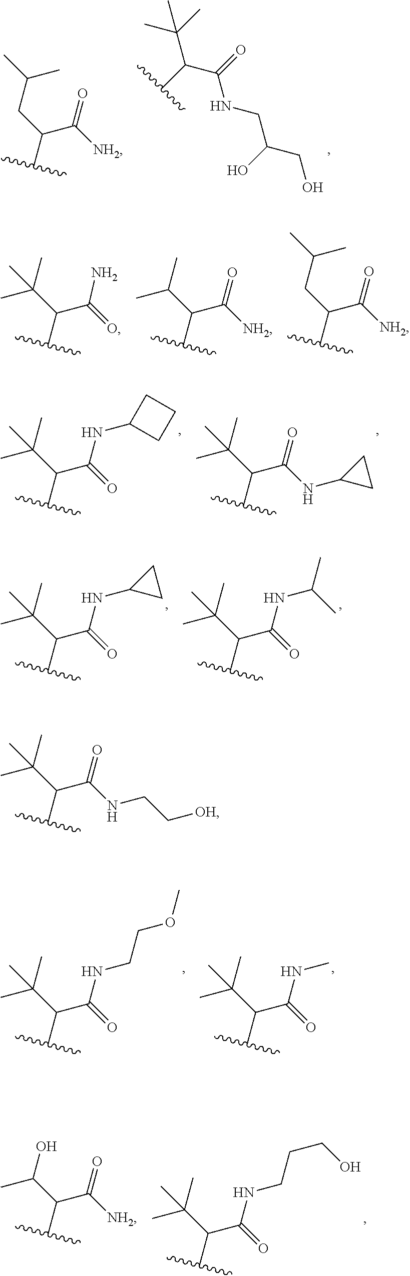 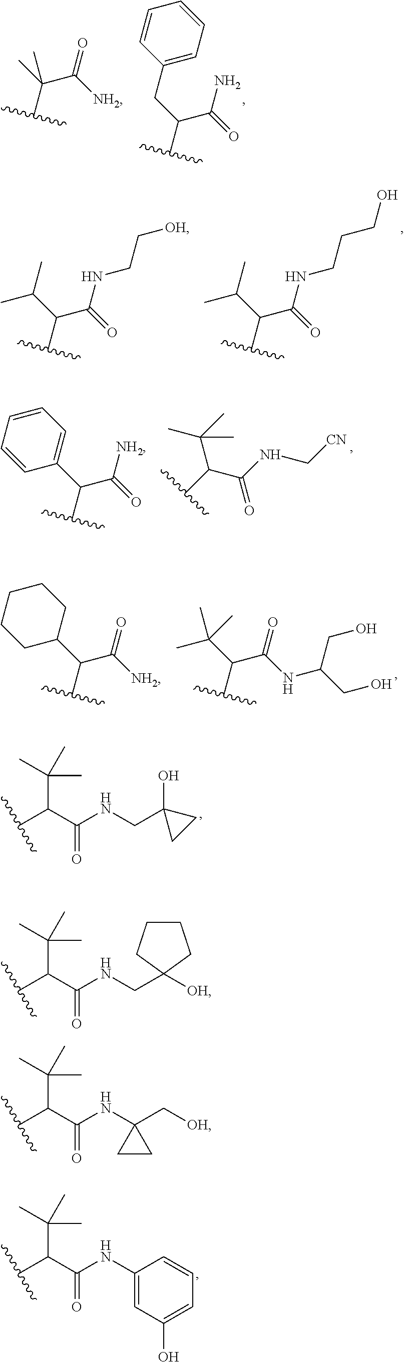 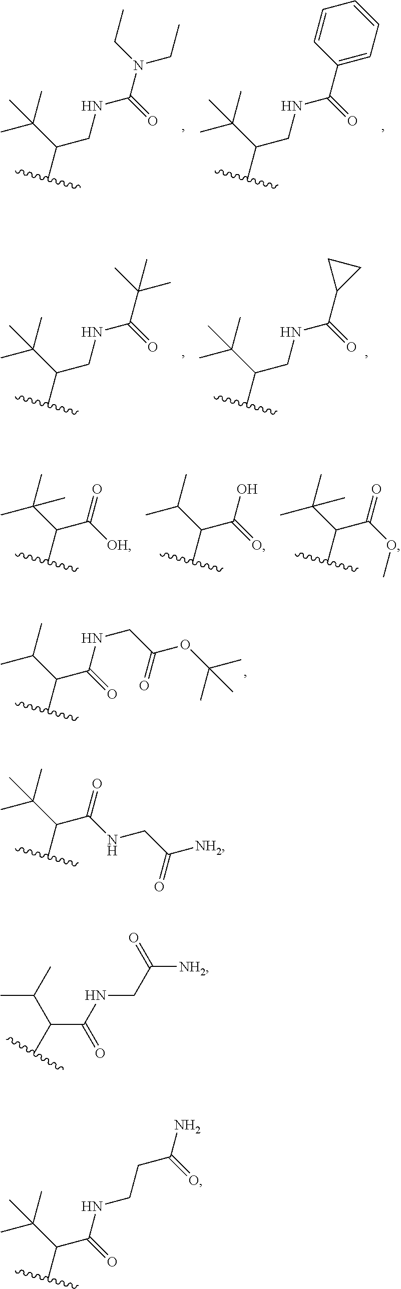 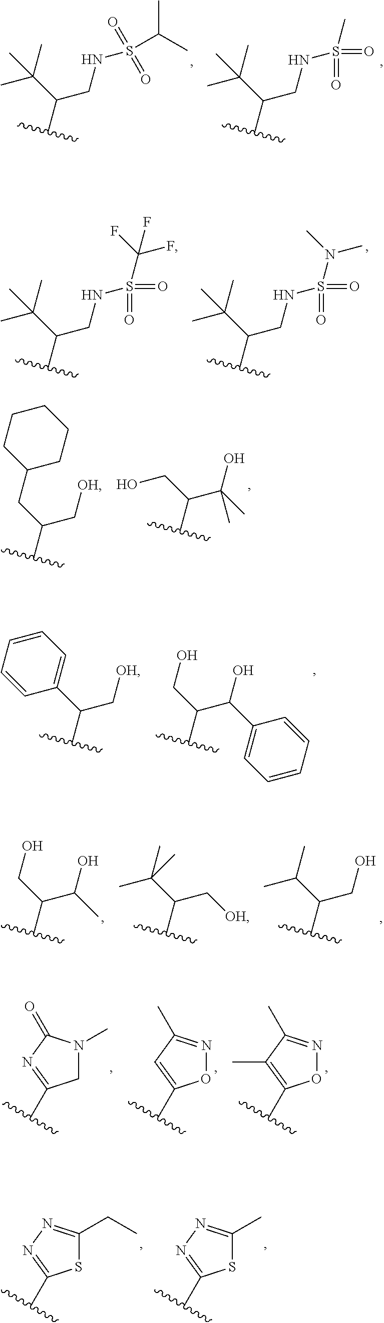 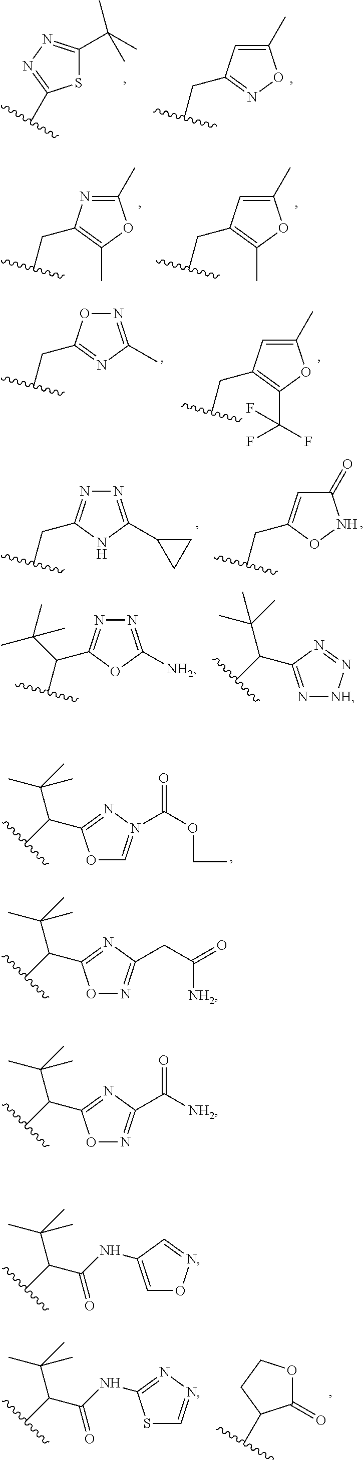 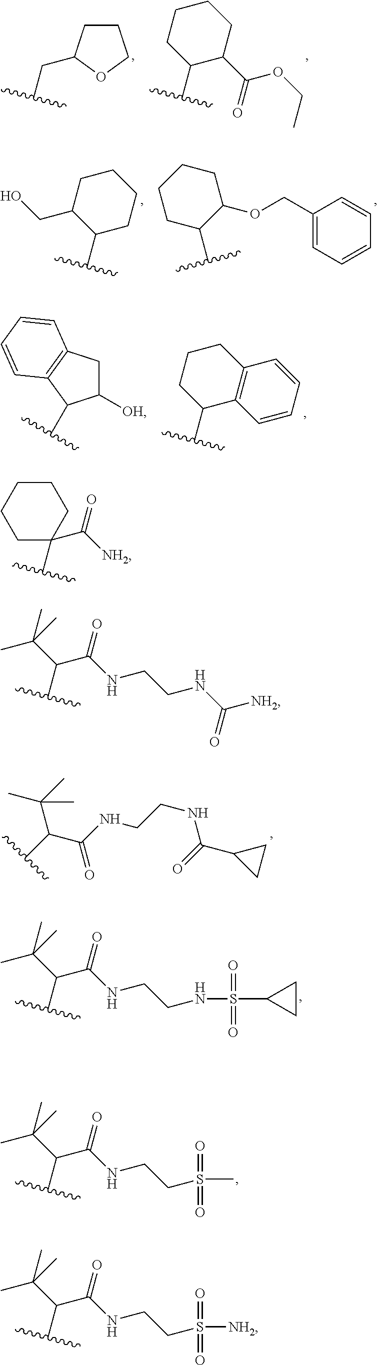 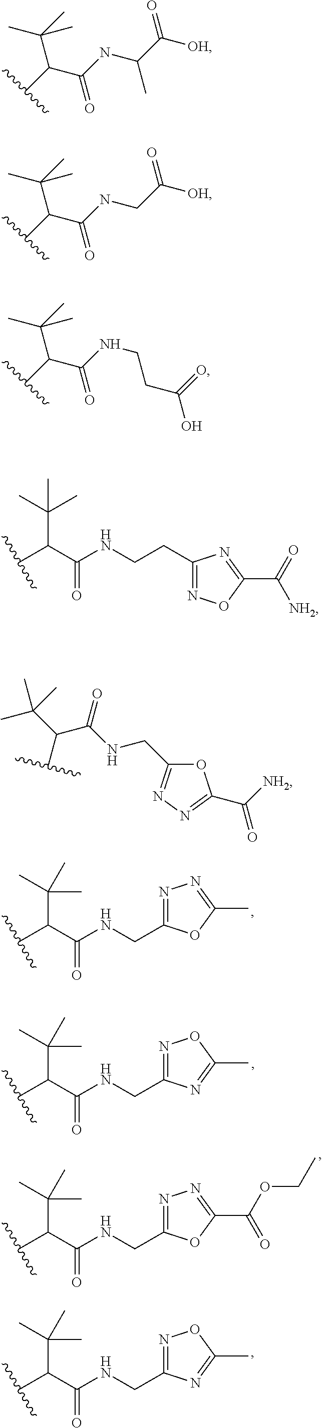 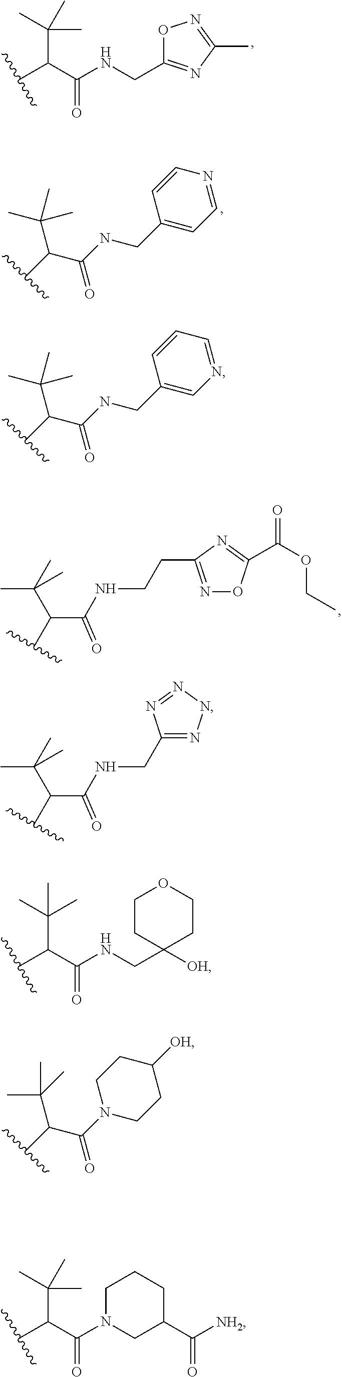 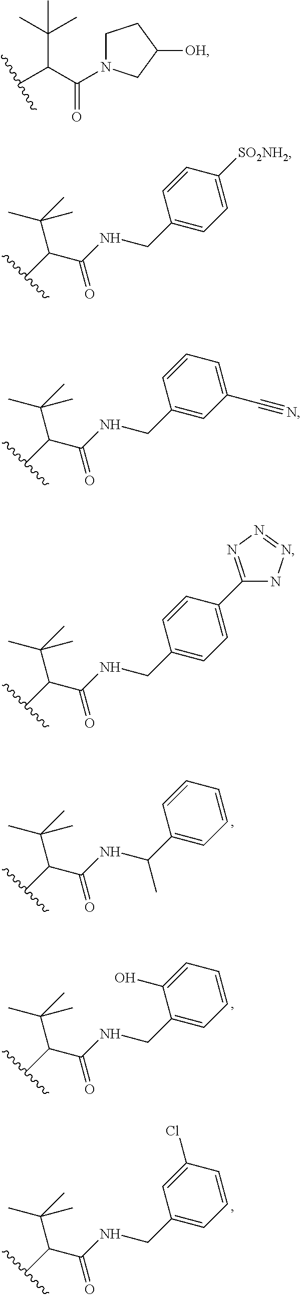 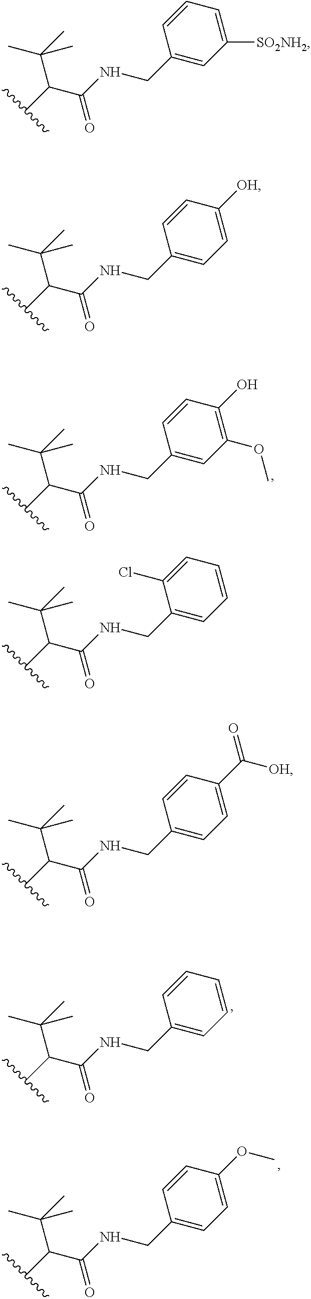 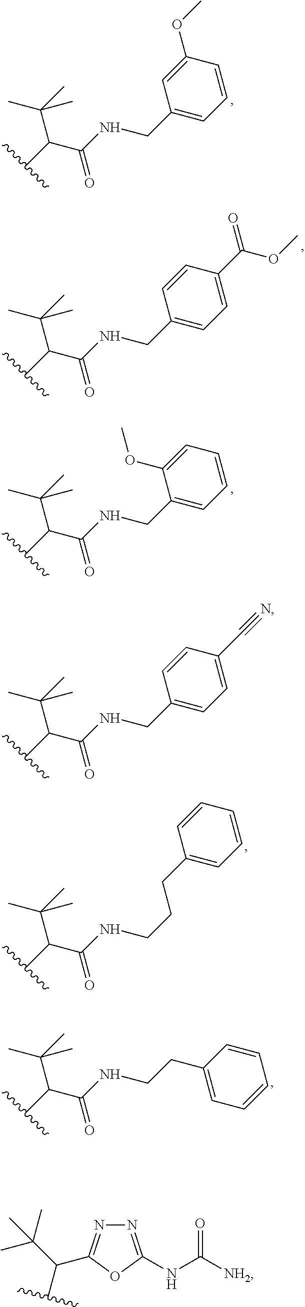 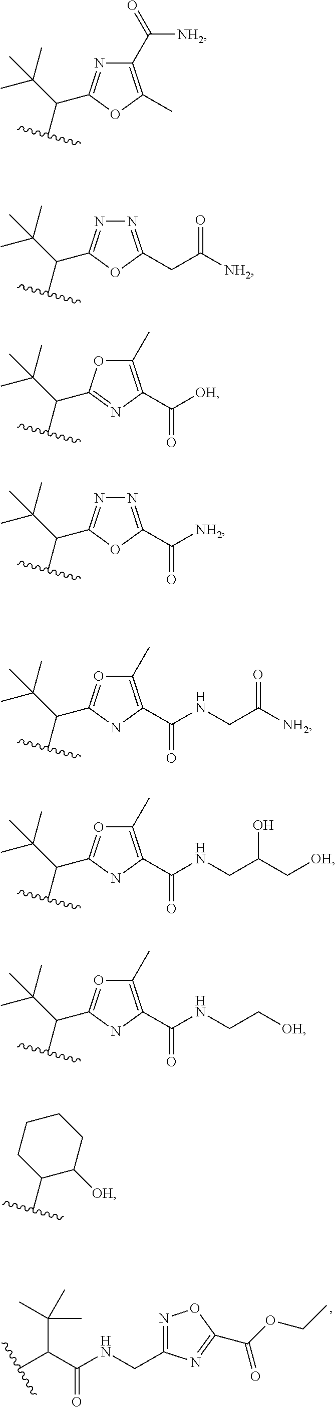 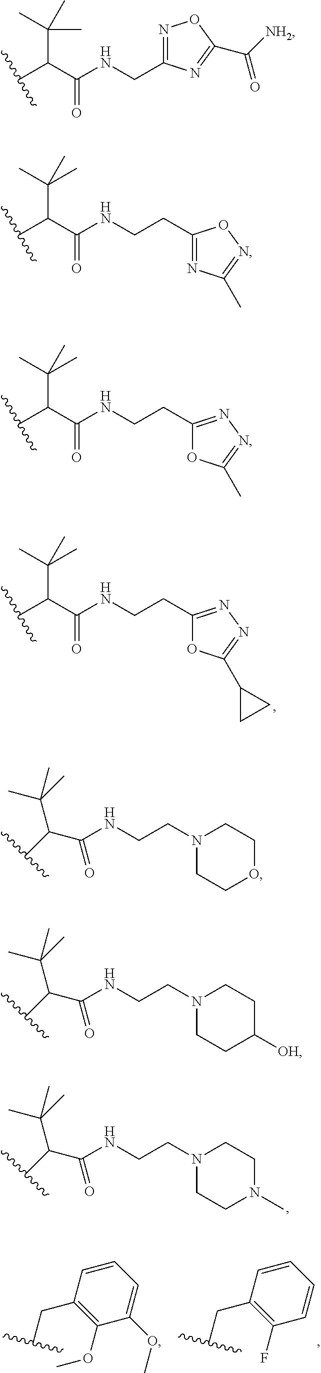 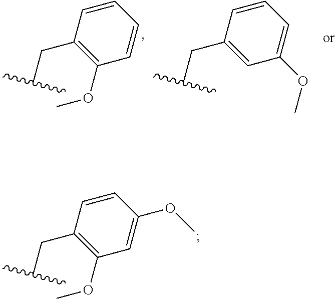
and
each R³ is independently H, CH₃, chloro, bromo, fluoro, phenyl, NH₂—C(O)—, CH₃O—, 3-pyridinyl, 4-pyridinyl, or 2-oxazolyl.

4. A compound of formula I: 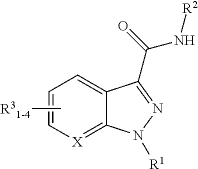
or a pharmaceutically acceptable salt thereof, wherein
X is CH or N;
R¹ is R⁴₁₋₅-aryl-(CH₂)ₙ— or R⁵₁₋₅-heteroaryl-(CH₂)ₙ—; wherein
each R⁴ is independently H, halo, cyano or NH₂—C(O)—;
each R⁵ is independently H or C₁-C₆ alkyl;
R² is NR¹¹R¹²—C(O)—R¹³CH—, R¹⁶—C(O)—R¹³CH—, NR¹⁷R¹⁸—C(O)—(CH₂)ₙ—NR¹⁹—C(O)—R¹³CH—, R²²R²³CH—, R²⁴₁₋₅-heteroaryl-R¹³CH—, R²⁶₁₋₅—C₃-C₇ cycloalkyl, NR²⁷R²⁸—(CH₂)ₙ—NR²⁹—C(O)—R¹³CH—, R³⁰—SO₂—NR³¹—(CH₂)ₙ—NR¹⁹—C(O)—R¹³CH—, R³⁹—SO₂—(CH₂)ₙ—NR³¹—C(O)—R¹³CH—, R³²—C(O)—R³³CH—NR³⁴—C(O)—R¹³CH—, R³⁵₁₋₅-heteroaryl-(CH₂)ₙ—NR³⁶—C(O)—R¹³CH—, R³⁷₁₋₅-heterocyclyl-(CH₂)ₙ—NR³⁶—C(O)—R¹³CH—, R³⁷₁₋₅-heterocyclyl-C(O)—R¹³CH— or R⁴¹₁₋₅-aryl-(CH₂)ₙ—; wherein
R¹¹ and R¹² are independently H, C₁-C₆ alkyl, OH—C₁-C₆ alkyl, (OH)₂—C₁-C₆ alkyl, C₁-C₆ alkoxy-(CH₂)ₙ—, C₃-C₇ cycloalkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl, OH-aryl,
R¹³ is H, C₁-C₆ alkyl, OH—C₁-C₆ alkyl, aryl, aryl-(CH₂)ₙ—, or C₃-C₇ cycloalkyl;
R¹⁶ is OH or C₁-C₆ alkoxy;
R¹⁷, R¹⁸ and R¹⁹ are independently H or C₁-C₆ alkyl;
R²² and R²³ are independently C₁-C₆ alkyl, C₃-C₇ cycloalkyl-(CH₂)ₙ—, OH—C₁-C₆ alkyl, or aryl;
each R²⁴ is independently H, C₁-C₆ alkyl, NH₂, NH₂—C(O)—NH—, NH₂—C(O)—, NH₂—C(O)—(CH₂)ₙ—, OH—C(O)—, NH₂—C(O)—(CH₂)ₙ—NH—C(O)—, (OH)₂—C₁-C₆ alkyl-NH—C(O)—, or OH—C₁-C₆ alkyl-NH—C(O)—;
each R²⁶ is independently H, OH, OH—C₁-C₆ alkyl, aryl-(CH₂)ₙ—O—, NH₂—C(O)— or C₁-C₆ alkoxy-C(O)—;
R²⁷ and R²⁸ independently are H or NH₂—C(O)—;
R²⁹ R³³, R³⁴, R³⁶ and R³⁸ are independently H or C₁-C₆ alkyl;
R³⁰ is C₁-C₆ alkyl, C₃-C₇ cycloalkyl or NH₂;
R³¹ is H,
R³² is OH;
each R³⁵ is independently H, C₁-C₆ alkyl, NH₂—C(O)—, C₁-C₆ alkoxy-C(O)—, or C₃-C₇ cycloalkyl;
each R³⁷ is independently H, NH₂C(O)— or OH;
each R⁴¹ is independently H, C₁-C₆ alkoxy or halo;
n is an integer from 1 to 6; and
each R³ is independently H, halo, C₁-C₆ alkyl, aryl, NH₂—C(O)—, C₁-C₆ alkoxy or heteroaryl.

5. The compound of claim 4 wherein
X is CH or N;
R¹ is R⁴₁₋₅-benzyl, R⁵₁₋₅-isoxazolyl-CH₂— or R⁵₁₋₅-pyridinyl-CH₂—; wherein
each R⁴ is H, fluoro, cyano, NH₂—C(O)—;
each R⁵ is independently H or CH₃;
R² is NR¹¹R¹²—C(O)—R¹³CH—, R¹⁶—C(O)—R¹³CH—, NR¹⁷R¹⁸—C(O)—CH₂—NR¹⁹—C(O)—R¹³CH—, NR¹⁷R¹⁸—C(O)— (CH₂)₂—NR¹⁹—C(O)—R¹³CH—, R²²R²³CH—, R²⁴₁₋₅-furyl-R¹³CH—, R²⁴₁₋₅-oxadiazolyl-R¹³CH—, R²⁴₁₋₅-tetrazolyl-R¹³CH—, R²⁶₁₋₅-cyclohexyl, R²⁶₁₋₅-tetrahydronapthyl, R²⁶₁₋₅-dihydroindenyl, NR²⁷R²⁸—(CH₂)₂—NR²⁹—C(O)—R¹³CH—, R³⁰—SO₂—NR³¹—(CH₂)₂—NR¹⁹—C(O)—R¹³CH—, R³⁰—SO₂—(CH₂)₂—NR³¹—C(O)—R¹³CH—, R³²—C(O)—R³³CH—NR³⁴—C(O)—R¹³CH—, R³⁵₁₋₅-oxadiazole-CH₂—NR³⁶—C(O)—R¹³CH—, R³⁵₁₋₅-oxadiazole-(CH₂)₂—NR³⁶—C(O)—R¹³CH—, R³⁷₁₋₅-morpholinyl-(CH₂)₂—NR³⁶—C(O)—R¹³CH—, R³⁷₁₋₅-piperidinyl-(CH₂)₂—NR³⁶—C(O)—R¹³CH—, R³⁷₁₋₅-piperazinyl-(CH₂)₂—NR³⁶—C(O)—R¹³CH—, R³⁷₁₋₅-tertrahydropyranyl-(CH₂)₂—NR³⁶—C(O)—R¹³CH—, R³⁷₁₋₅-piperidinyl-C(O)—R¹³CH—, R³⁷₁₋₅-pyrrolidinyl-C(O)—R¹³CH— or R⁴¹₁₋₅-benzyl; wherein
R¹¹ and R¹² are independently H, CH₃, (CH₃)₂CH—, cyclobutyl, cyclopropyl, CH₃—O—(CH₂)₂—, OH-ethyl, OH-propyl, (OH)₂-propyl, (OH—CH₂)₂—CH—, OH-cyclopropyl-CH₂—, OH-cyclopentyl-CH₂—, OH—CH₂-cyclopropyl, or OH-phenyl;
R¹³ is H, (CH₃)₃C, (CH₃)₂CHCH₂—, (CH₃)₂CH—, OH-ethyl, benzyl, phenyl, or cyclohexyl;
R¹⁶ is OH or CH₃O;
R¹⁷, R¹⁸ and R¹⁹ are independently H or CH₃;
R²² and R²³ are independently (CH₃)₃C—, (CH₃)₂CH—, cyclohexyl-CH₂—, OHCH₂, phenyl, OH-isopropyl, or OH-ethyl;
each R²⁴ is independently H, CH₃, NH₂, NH₂—C(O)—NH—, NH₂—C(O)—, NH₂—C(O)—CH₂—, OH—C(O)—, NH₂—C(O)—CH₂—NH—C(O)—, (OH)₂-propyl-NH—C(O)—, or OH-ethyl-NH—C(O)—;
each R²⁶ is independently H, OH, OHCH₂, benzyl-O—, NH₂—C(O)— or CH₃CH₂—O—C(O)—;
R²⁷ and R²⁸ are independently H or NH₂—C(O)—;
R²⁹R³³, R³⁴, R³⁶ and R³⁸ are independently H or CH₃;
R³⁰ is CH₃, cyclopropyl or NH₂;
R³¹ is H,
R³² is OH;
each R³⁵ is independently H, CH₃, NH₂—C(O)—, CH₃CH₂—O—C(O)—, or cyclopropyl;
each R³⁷ is independently H, NH₂C(O)— or OH;
each R⁴¹ is independently H, CH₃O or fluoro; and
each R³ is independently H, CH₃, chloro, bromo, fluoro, phenyl, NH₂—C(O)—, CH₃O, pyridinyl or oxazolyl.

6. The compound of claim 5 wherein
X is CH or N;
R¹ is 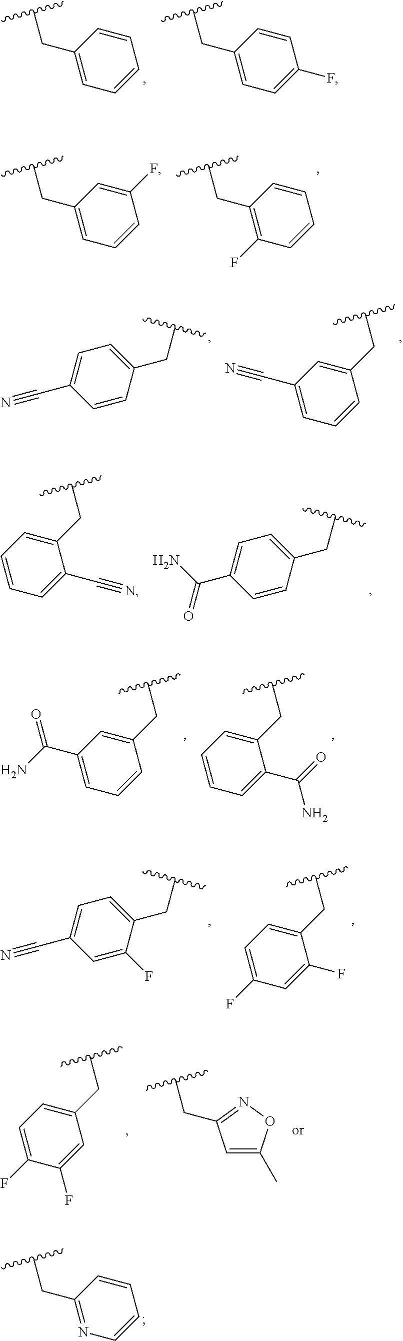
R² is 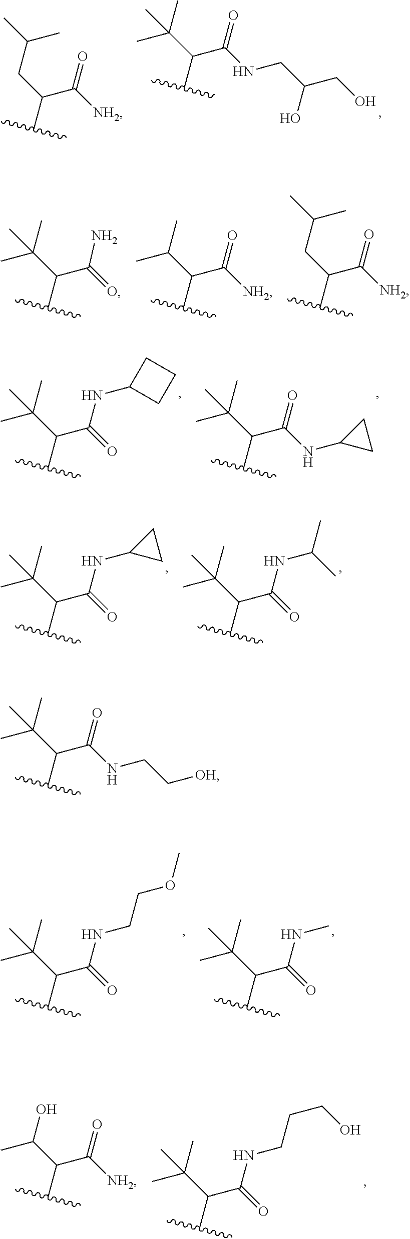 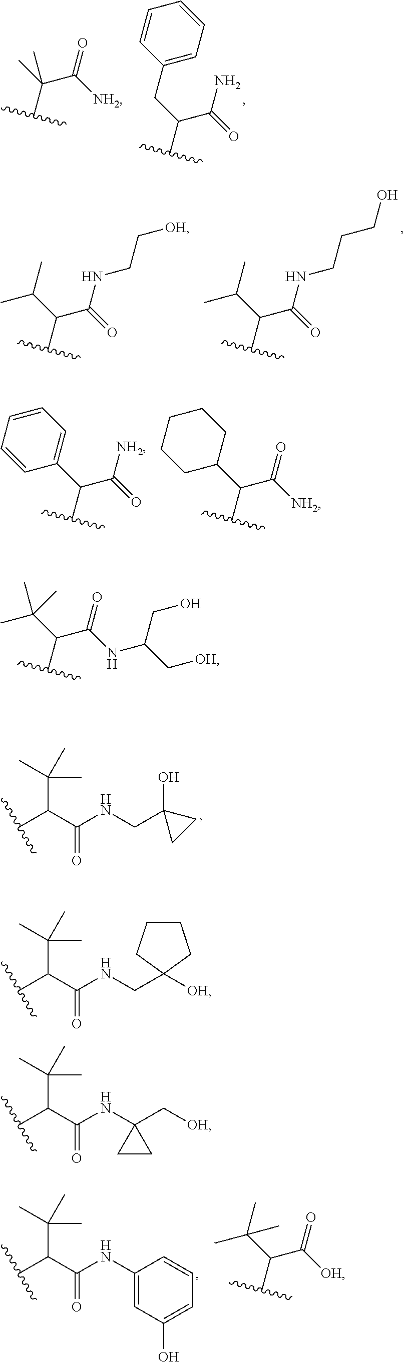 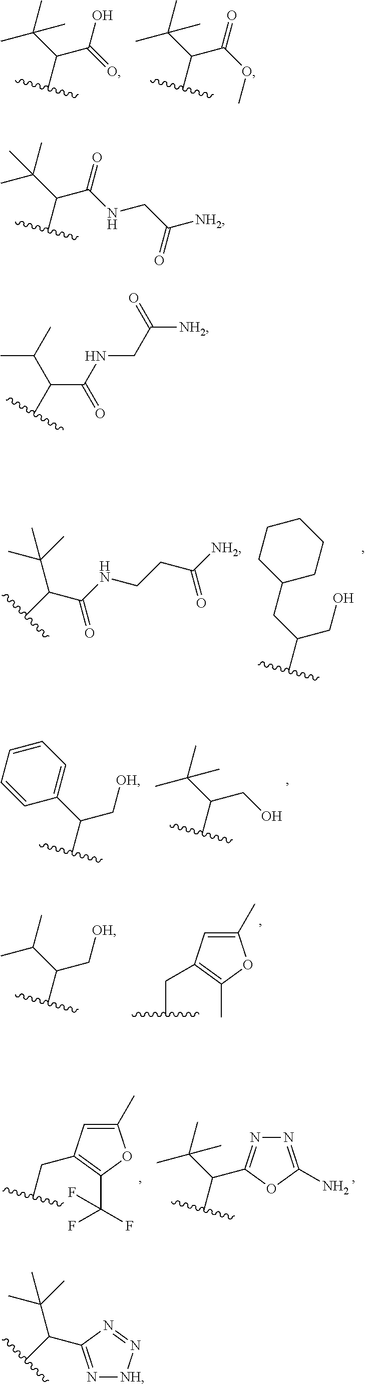 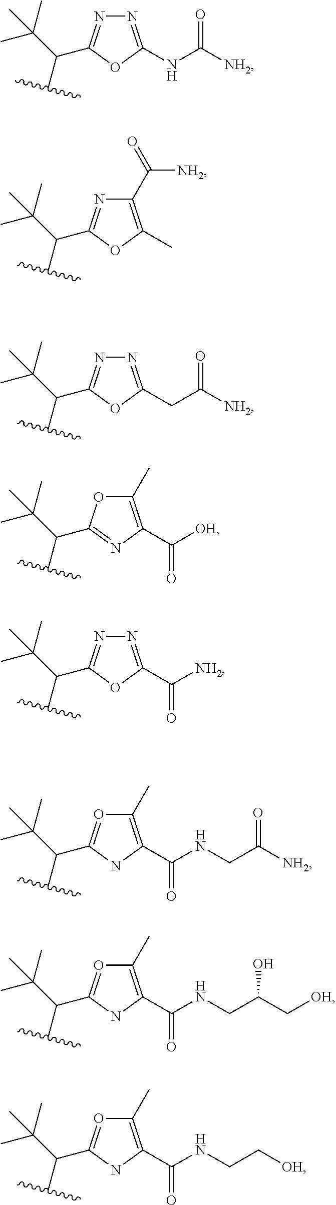 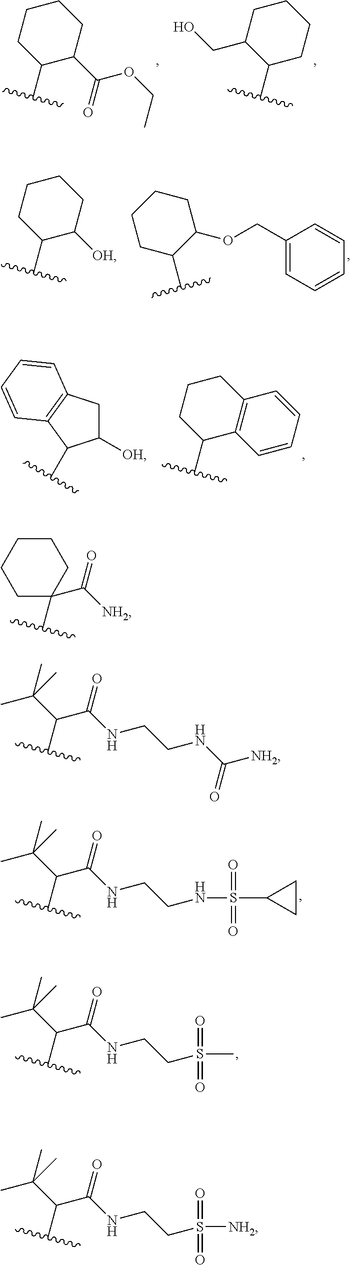 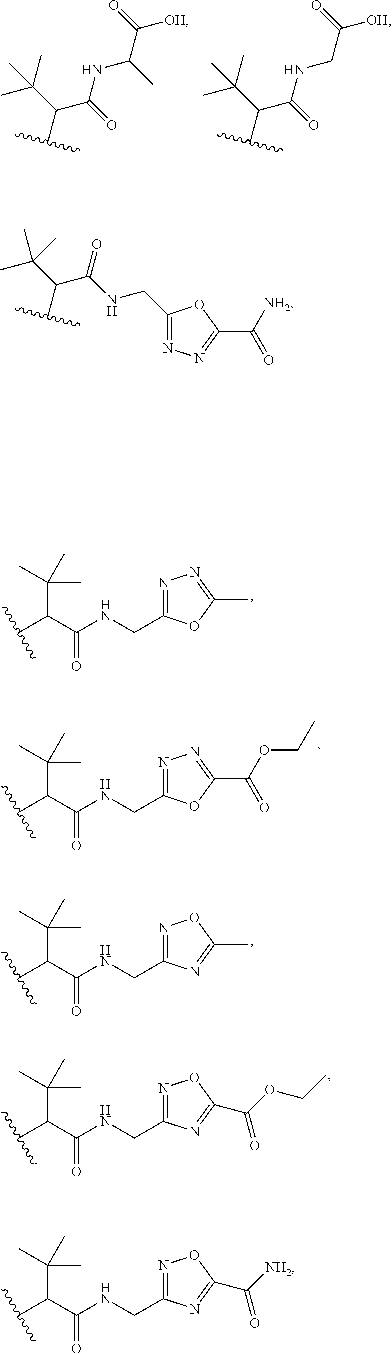 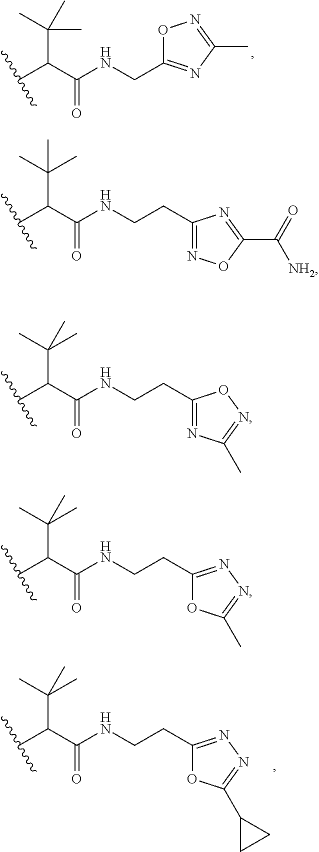 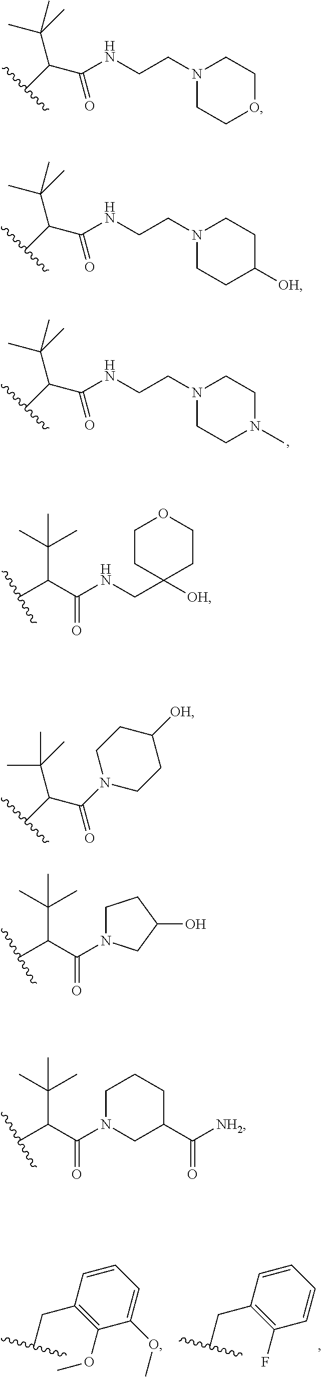 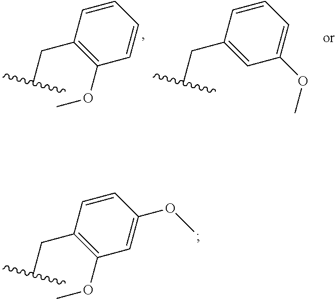
and
each R³ is independently H, CH₃, chloro, bromo, fluoro, phenyl, NH₂—C(O)—, CH₃O, 3-pyridinyl, 4-pyridinyl, or 2-oxazolyl.

7. The compound of claim 4 wherein X is CH.

8. The compound of claim 7 wherein
X is CH;
R¹ is R⁴₁₋₅-aryl-(CH₂)ₙ— or R⁵₁₋₅-heteroaryl-(CH₂)—; wherein
each R⁴ is independently H, halo, cyano, or NH₂—C(O)—;
each R⁵ is independently H or C₁-C₆ alkyl;
R2 is NR¹¹R¹²—C(O)—R¹³CH—, NR¹⁷R¹⁸—C(O)—(CH₂)ₙ—NR¹⁹—C(O)—R¹³CH—, R²²R²³CH—, R²⁴₁₋₅-heteroaryl-R¹³CH, R³⁰—SO₂—NR³¹—(CH₂)ₙ—NR¹⁹—C(O)—R¹³CH—, R³⁰—SO₂—(CH₂)ₙ—NR³¹—C(O)—R¹³CH— or R³²—C(O)—R³³CH—NR³⁴—C(O)—R¹³CH—; wherein
R¹¹ and R¹² are independently H, OH—C₁-C₆ alkyl, (OH)₂—C₁-C₆ alkyl, C₃-C₇ cycloalkyl or (OH—C₁-C₆ alkyl)₂-(CH₂)ₙ—;
R¹³ is C₁-C₆ alkyl;
R¹⁷, R¹⁸ and R¹⁹ are independently H;
R²² and R²³ are independently C₁-C₆ alkyl or OH—C₁-C₆ alkyl;
each R²⁴ is independently Hor NH₂;
R³⁰ is C₃-C₇ cycloalkyl or NH₂;
R³¹ is H;
R³² is OH;
R³³ is H;
R³⁴ is H;
n is an integer from 1 to 6; and
R³ is H, halo or C₁-C₆ alkyl;

9. The compound of claim 8 wherein
X is CH;
R¹ is 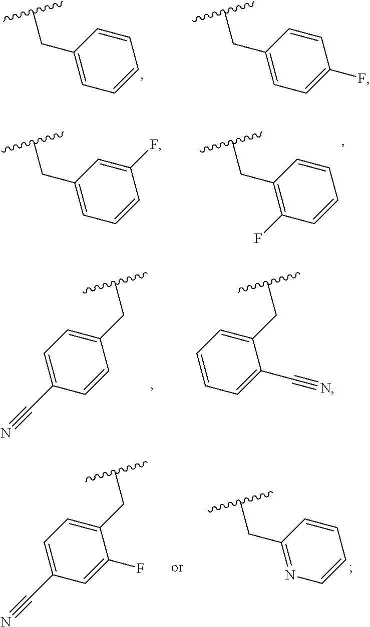
R² is 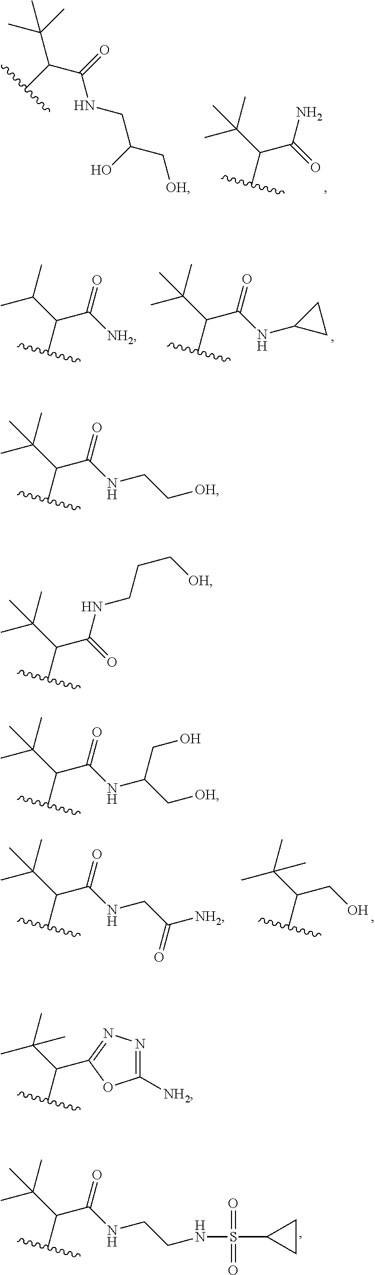 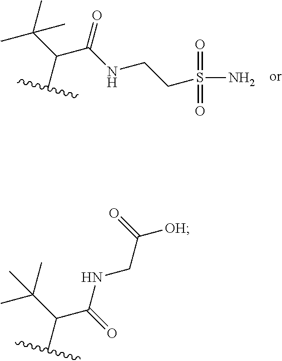
and
R³ is H, F, C₁ or CH₃;

10. The compound of claim 4 wherein
X is N;
R¹ is R⁴₁₋₅-aryl-(CH₂)ₙ— or R⁵₁₋₅-heteroaryl-(CH₂)ₙ—; wherein
each R⁴ is independently H, halo, cyano, or NH₂—C(O)—;
each R⁵ is independently H;
R² is NR¹¹R¹²—C(O)—R¹³CH—, R²²R²³CH— or R¹⁶—C(O)—R¹³CH—; wherein
R¹¹ and R¹² are independently H;
R¹³ is C₁-C₆ alkyl or OH—C₁-C₆ alkyl;
R¹⁶ is OH;
R²² and R²³ are independently C₁-C₆ alkyl or OH—C₁-C₆ alkyl;
n is an integer from 1 to 6; and
R³ is H.

11. The compound of claim 10 wherein
X is N;
R¹ is R⁴₁₋₅-benzyl or R⁵₁₋₅-pyridinyl-CH₂—; wherein
each R⁴ is H or fluoro;
each R⁵ is independently H;
R² is NR¹¹R¹²—C(O)—R¹³CH—, R²²R²³CH— or R¹⁶—C(O)—R¹³CH—; wherein
R¹¹ and R¹² are independently H;
R¹³ is (CH₃)₃C, (CH₃)₂CHCH₂, (CH₃)₂CH₂OH-ethyl;
R¹⁶ is OH;
R²² and R²³ are independently (CH₃)₃C or OHCH₂; and
R³ is H.

12. The compound of claim 11 wherein
X is N;
R¹ is 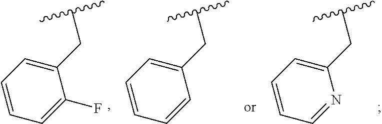
R² is 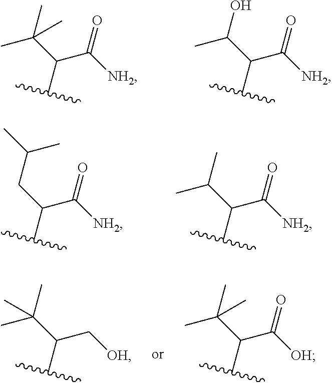
and
R³ is H.

13. A compound, or a pharmaceutically acceptable salt thereof, selected from the group consisting of
N-[(1S)-1-(aminocarbonyl)-2,2-dimethylpropyl]-1-benzyl-5-bromo-1H-indazole-3-carboxamide;
1-[4-(aminocarbonyl)benzyl]-N-[(1S)-1-(aminocarbonyl)-2,2-dimethylpropyl]-1H-indazole-3-carboxamide;
N-[(1S)-1-(aminocarbonyl)-2,2-dimethylpropyl]-1-benzyl-5-pyridin-3-yl-1H-indazole-3-carboxamide;
1-[3-(aminocarbonyl)benzyl]-N-[(1S)-1-(aminocarbonyl)-2,2-dimethylpropyl]-1H-indazole-3-carboxamide;
N-[(1S)-1-(aminocarbonyl)-2,2-dimethylpropyl]-1-benzyl-6-bromo-1H-indazole-3-carboxamide;
1-[2-(aminocarbonyl)benzyl]-N-[(1S)-1-(aminocarbonyl)-2,2-dimethylpropyl]-1H-indazole-3-carboxamide;
N-[(1S)-1-(aminocarbonyl)-2,2-dimethylpropyl]-1-benzyl-5-(1,3-oxazol-2-yl)-1H-indazole-3-carboxamide;
N-[(1S)-1-(aminocarbonyl)-2,2-dimethylpropyl]-1-benzyl-5-pyridin-4-yl-1H-indazole-3-carboxamide;
N-[(1S)-1-(aminocarbonyl)-2,2-dimethylpropyl]-1-benzyl-6-pyridin-4-yl-1H-indazole-3-carboxamide;
methyl N-[(1-benzyl-1H-indazol-3-yl)carbonyl]-3-methyl-L-valinate;
1-benzyl-N-(4-methoxybenzyl)-1H-indazole-3-carboxamide;
1-benzyl-N-(2-methoxybenzyl)-1H-indazole-3-carboxamide;
1-benzyl-N-(2-fluorobenzyl)-1H-indazole-3-carboxamide;
1-benzyl-N-(2,3-dimethoxybenzyl)-1H-indazole-3-carboxamide;
1-benzyl-N-(3-methoxybenzyl)-1H-indazole-3-carboxamide;
N-[(1-benzyl-1H-indazol-3-yl)carbonyl]-3-methyl-L-valine;
N-[(1S)-1-(aminocarbonyl)-2,2-dimethylpropyl]-1-benzyl-6-pyridin-3-yl-1H-indazole-3-carboxamide;
N-[(1S)-1-(aminocarbonyl)-2,2-dimethylpropyl]-1-benzyl-5-methoxy-1H-indazole-3-carboxamide;
N˜3˜-[(1S)-1-(aminocarbonyl)-2,2-dimethylpropyl]-1-benzyl-1H-indazole-3,5-dicarboxamide;
N-[(1S)-1-(aminocarbonyl)-2,2-dimethylpropyl]-1-benzyl-6-phenyl-1H-indazole-3-carboxamide;
N-[(1S)-1-(aminocarbonyl)-2,2-dimethylpropyl]-1-benzyl-5-phenyl-1H-indazole-3-carboxamide;
1-(4-cyanobenzyl)-N-{(1S)-1-[(cyclopropylamino)carbonyl]-2,2-dimethylpropyl}-1H-indazole-3-carboxamide;
N-{[1-(4-cyanobenzyl)-1H-indazol-3-yl]carbonyl}-3-methyl-L-valylglycinamide;
1-(4-cyanobenzyl)-N-[(1S)-1-{[(3-hydroxypropyl)amino]carbonyl}-2,2-dimethylpropyl]-1H-indazole-3-carboxamide;
1-(4-cyanobenzyl)-N-[(2,5-dimethyl-3-furyl)methyl]-1H-indazole-3-carboxamide;
1-(4-cyanobenzyl)-N-[(1S)-1-{[(2-hydroxyethyl)amino]carbonyl}-2,2-dimethylpropyl]-1H-indazole-3-carboxamide;
1-(4-cyanobenzyl)-N-[(1S)-2,2-dimethyl-1-(2H-tetrazol-5-yl)propyl]-1H-indazole-3-carboxamide;
N-[(1S)-1-(5-amino-1,3,4-oxadiazol-2-yl)-2,2-dimethylpropyl]-1-(4-cyanobenzyl)-1H-indazole-3-carboxamide;
N-{[1-(4-cyanobenzyl)-1H-indazol-3-yl]carbonyl}-3-methyl-L-valine;
1-benzyl-N-[(1S)-1-({[(2S)-2,3-dihydroxypropyl]amino}carbonyl)-2,2-dimethylpropyl] 1H-indazole-3-carboxamide;
1-benzyl-N-[(1S)-1-({[(2R)-2,3-dihydroxypropyl]amino}carbonyl)-2,2-dimethylpropyl]-1H-indazole-3-carboxamide;
1-benzyl-N-[(1S)-1-{5-[(cyclopropylcarbonyl)amino]-1,3,4-oxadiazol-2-yl}-2,2-dimethylpropyl]-1H-indazole-3-carboxamide;
N-[(1-benzyl-1H-indazol-3-yl)carbonyl]-3-methyl-L-valylglycine;
N-[(1S)-1-({[(2R)-2,3-dihydroxypropyl]amino}carbonyl)-2,2-dimethylpropyl]-1-(4-fluorobenzyl)-1H-indazole-3-carboxamide;
N-[(1S)-1-{5-[(cyclopropylcarbonyl)amino]-1,3,4-oxadiazol-2-yl}-2,2-dimethylpropyl]-1-(4-fluorobenzyl)-1H-indazole-3-carboxamide;
N-[(1S)-1-({[(2S)-2,3-dihydroxypropyl]amino}carbonyl)-2,2-dimethylpropyl]-1-(4-fluorobenzyl)-1H-indazole-3-carboxamide;
N-{(1S)-1-[(cyclopropylamino)carbonyl]-2,2-dimethylpropyl}-1-(4-fluorobenzyl)-1H-indazole-3-carboxamide;
1-(4-fluorobenzyl)-N-[(1S)-1-{[(2-hydroxyethyl)amino]carbonyl}-2,2-dimethylpropyl]-1H-indazole-3-carboxamide;
N-{[1-(4-fluorobenzyl)-1H-indazol-3-yl]carbonyl}-3-methyl-L-valylglycinamide;
N-{[1-(4-fluorobenzyl)-1H-indazol-3-yl]carbonyl}-3-methyl-L-valylglycine;
N-{(1S)-1-[({2-[(aminocarbonyl)amino]ethyl}amino)carbonyl]-2,2-dimethylpropyl}-1-benzyl-1H-indazole-3-carboxamide;
N-{(1S)-1-[({2-[(aminocarbonyl)amino]ethyl}amino)carbonyl]-2,2-dimethylpropyl}-1-(4-cyanobenzyl)-1H-indazole-3-carboxamide;
N-{(1S)-1-[({2-[(aminocarbonyl)amino]ethyl}amino)carbonyl]-2,2-dimethylpropyl}-1-(4-fluorobenzyl)-1H-indazole-3-carboxamide;
N-[(1S)-1-(aminocarbonyl)-2,2-dimethylpropyl]-1-(4-cyano-2-fluorobenzyl)-1H-indazole-3-carboxamide;
1-(4-cyano-2-fluorobenzyl)-N-{(1S)-1-[(cyclopropylamino)carbonyl]-2,2-dimethylpropyl}-1H-indazole-3-carboxamide;
1-(4-cyano-2-fluorobenzyl)-N-[(1S)-1-{5-[(cyclopropylcarbonyl)amino]-1,3,4-oxadiazol-2-yl}-2,2-dimethylpropyl]-1H-indazole-3-carboxamide;
1-(4-cyano-2-fluorobenzyl)-N-[(1S)-1-({[(2R)-2,3-dihydroxypropyl]amino}carbonyl)-2,2-dimethylpropyl]-1H-indazole-3-carboxamide;
1-(4-cyano-2-fluorobenzyl)-N-[(1S)-1-({[(2S)-2,3-dihydroxypropyl]amino}carbonyl)-2,2-dimethylpropyl]-1H-indazole-3-carboxamide;
1-(4-cyano-2-fluorobenzyl)-N-[(1S)-1-{[(2-hydroxyethyl)amino]carbonyl}-2,2-dimethylpropyl]-1H-indazole-3-carboxamide;
N-{[1-(4-cyano-2-fluorobenzyl)-1H-indazol-3-yl]carbonyl}-3-methyl-L-valylglycinamide;
1-(4-cyano-2-fluorobenzyl)-N-[(1S)-1-{[(3-hydroxypropyl)amino]carbonyl}-2,2-dimethylpropyl]-1H-indazole-3-carboxamide;
N-{(1S)-1-[({2-[(aminocarbonyl)amino]ethyl}amino)carbonyl]-2,2-dimethylpropyl}-1-(4-cyano-2-fluorobenzyl)-1H-indazole-3-carboxamide;
N-[(1S)-1-(5-amino-1,3,4-oxadiazol-2-yl)-2,2-dimethylpropyl]-1-(4-cyano-2-fluorobenzyl)-1H-indazole-3-carboxamide;
1-benzyl-N-{(1S)-1-[({2-[(cyclopropylsulfonyl)amino]ethyl}amino)carbonyl]-2,2-dimethylpropyl}-1H-indazole-3-carboxamide;
1-(4-cyanobenzyl)-N-{(1S)-1-[({2-[(cyclopropylsulfonyl)amino]ethyl}amino)carbonyl]-2,2-dimethylpropyl}-1H-indazole-3-carboxamide;
1-(4-cyano-2-fluorobenzyl)-N-{(1S)-1-[({2-[(cyclopropylsulfonyl)amino]ethyl}amino)carbonyl]-2,2-dimethylpropyl}-1H-indazole-3-carboxamide;
N-{(1S)-1-[({2-[(cyclopropylsulfonyl)amino]ethyl}amino)carbonyl]-2,2-dimethylpropyl}-1-(4-fluorobenzyl)-1H-indazole-3-carboxamide;
1-benzyl-N-{(1S)-1-[({2-[(cyclopropylcarbonyl)amino]ethyl}amino)carbonyl]-2,2-dimethylpropyl}-1H-indazole-3-carboxamide;
1-(4-cyanobenzyl)-N-{(1S)-1-[({2-[(cyclopropylcarbonyl)amino]ethyl}amino)carbonyl]-2,2-dimethylpropyl}-1H-indazole-3-carboxamide;
1-(4-cyano-2-fluorobenzyl)-N-{(1S)-1-[({2-[(cyclopropylcarbonyl)amino]ethyl}amino)carbonyl]-2,2-dimethylpropyl}-1H-indazole-3-carboxamide;
N-{(1S)-1-[({2-[(cyclopropylcarbonyl)amino]ethyl}amino)carbonyl]-2,2-dimethylpropyl}-1-(4-fluorobenzyl)-1H-indazole-3-carboxamide;
1-(4-cyanobenzyl)-N-[(1S)-2,2-dimethyl-1-({[2-(methylsulfonyl)ethyl]amino}carbonyl)propyl]-1H-indazole-3-carboxamide;
1-(4-cyano-2-fluorobenzyl)-N-[(1S)-2,2-dimethyl-1-({[2-(methylsulfonyl)ethyl]amino}carbonyl)propyl]-1H-indazole-3-carboxamide;
N-[(1S)-1-({[2-(aminosulfonyl)ethyl]amino}carbonyl)-2,2-dimethylpropyl]-1-(4-cyanobenzyl)-1H-indazole-3-carboxamide;
N-[(1S)-1-({[2-(aminosulfonyl)ethyl]amino}carbonyl)-2,2-dimethylpropyl]-1-(4-cyano-2-fluorobenzyl)-1H-indazole-3-carboxamide;
1-(4-cyanobenzyl)-N-{(1S)-1-[(cyclopropylamino)carbonyl]-2,2-dimethylpropyl}-7-fluoro-1H-indazole-3-carboxamide;
1-(4-cyanobenzyl)-7-fluoro-N-[(1S)-1-{[(2-hydroxyethyl)amino]carbonyl}-2,2-dimethylpropyl]-1H-indazole-3-carboxamide;
1-(4-cyanobenzyl)-7-fluoro-N-[(1S)-1-{[(3-hydroxypropyl)amino]carbonyl}-2,2-dimethylpropyl]-1H-indazole-3-carboxamide;
N-{[1-(4-cyanobenzyl)-7-fluoro-1H-indazol-3-yl]carbonyl}-3-methyl-L-valylglycinamide;
N-[(1S)-1-(aminocarbonyl)-2,2-dimethylpropyl]-1-(4-cyanobenzyl)-7-fluoro-1H-indazole-3-carboxamide;
N-[(1S)-1-(5-amino-1,3,4-oxadiazol-2-yl)-2,2-dimethylpropyl]-1-(4-cyanobenzyl)-7-fluoro-1H-indazole-3-carboxamide;
1-(4-cyanobenzyl)-N-[(1S)-1-({[(2S)-2,3-dihydroxypropyl]amino}carbonyl)-2,2-dimethylpropyl]-7-fluoro-1H-indazole-3-carboxamide;
1-(4-cyanobenzyl)-N-[(1S)-1-({[(2R)-2,3-dihydroxypropyl]amino}carbonyl)-2,2-dimethylpropyl]-7-fluoro-1H-indazole-3-carboxamide;
1-(4-cyanobenzyl)-N-{(1S)-1-[({2-[(cyclopropylsulfonyl)amino]ethyl}amino)carbonyl]-2,2-dimethylpropyl}-7-fluoro-1H-indazole-3-carboxamide
N-{(1S)-1-[({[5-(aminocarbonyl)-1,3,4-oxadiazol-2-yl]methyl}amino)carbonyl]-2,2-dimethylpropyl}-1-(4-cyanobenzyl)-7-fluoro-1H-indazole-3-carboxamide;
1-(4-cyanobenzyl)-7-fluoro-N-[(1S)-1-({[2-hydroxy-1-(hydroxymethyl)ethyl]amino}carbonyl)-2,2-dimethylpropyl]-1H-indazole-3-carboxamide;
N-[(1S)-1-{5-[(aminocarbonyl)amino]-1,3,4-oxadiazol-2-yl}-2,2-dimethylpropyl]-1-(4-fluorobenzyl)-1H-indazole-3-carboxamide;
N-{(1S)-1-[4-(aminocarbonyl)-5-methyl-1,3-oxazol-2-yl]-2,2-dimethylpropyl}-1-(4-fluorobenzyl)-1H-indazole-3-carboxamide;
N-{(1S)-1-[5-(2-amino-2-oxoethyl)-1,3,4-oxadiazol-2-yl]-2,2-dimethylpropyl}-1-(4-fluorobenzyl)-1H-indazole-3-carboxamide;
2-[(1S)-1-({[1-(4-fluorobenzyl)-1H-indazol-3-yl]carbonyl}amino)-2,2-dimethylpropyl]-5-methyl-1,3-oxazole-4-carboxylic acid;
N-{(1S)-1-[5-(aminocarbonyl)-1,3,4-oxadiazol-2-yl]-2,2-dimethylpropyl}-1-(4-fluorobenzyl)-1H-indazole-3-carboxamide;
N-[(1S)-1-(4-{[(2-amino-2-oxoethyl)amino]carbonyl}-5-methyl-1,3-oxazol-2-yl)-2,2-dimethylpropyl]-1-(4-fluorobenzyl)-1H-indazole-3-carboxamide;
N-{(1S)-1-[4-({[(2S)-2,3-dihydroxypropyl]amino}carbonyl)-5-methyl-1,3-oxazol-2-yl]-2,2-dimethylpropyl}-1-(4-fluorobenzyl)-1H-indazole-3-carboxamide;
1-(4-fluorobenzyl)-N-[(1S)-1-(4-{[(2-hydroxyethyl)amino]carbonyl}-5-methyl-1,3-oxazol-2-yl)-2,2-dimethylpropyl]-1H-indazole-3-carboxamide;
N-[(1S)-2,2-dimethyl-1-({[(5-methyl-1,3,4-oxadiazol-2-yl)methyl]amino}carbonyl)propyl]-1-(4-fluorobenzyl)-1H-indazole-3-carboxamide;
1-(4-cyanobenzyl)-N-[(1S)-2,2-dimethyl-1-({[(5-methyl-1,3,4-oxadiazol-2-yl)methyl]amino}carbonyl)propyl]-1H-indazole-3-carboxamide;
ethyl 5-{[(N-{[1-(4-fluorobenzyl)-1H-indazol-3-yl]carbonyl}-3-methyl-L-valyl)amino]methyl}-1,3,4-oxadiazole-2-carboxylate;
ethyl 5-{[(N-{[1-(4-cyanobenzyl)-1H-indazol-3-yl]carbonyl}-3-methyl-L-valyl)amino]methyl}-1,3,4-oxadiazole-2-carboxylate;
N-{(1S)-1-[({[5-(aminocarbonyl)-1,3,4-oxadiazol-2-yl]methyl}amino)carbonyl]-2,2-dimethylpropyl}-1-(4-fluorobenzyl)-1H-indazole-3-carboxamide;
N-{(1S)-1-[({[5-(aminocarbonyl)-1,3,4-oxadiazol-2-yl]methyl}amino)carbonyl]-2,2-dimethylpropyl}-1-(4-cyanobenzyl)-1H-indazole-3-carboxamide;
N-[(1S)-2,2-dimethyl-1-({[(5-methyl-1,2,4-oxadiazol-3-yl)methyl]amino}carbonyl)propyl]-1-(4-fluorobenzyl)-1H-indazole-3-carboxamide;
1-(4-cyanobenzyl)-N-[(1S)-2,2-dimethyl-1-({[(5-methyl-1,2,4-oxadiazol-3-yl)methyl]amino}carbonyl)propyl]-1H-indazole-3-carboxamide;
1-(4-fluorobenzyl)-N-{(1S)-1-[(4-hydroxypiperidin-1-yl)carbonyl]-2,2-dimethylpropyl}-1H-indazole-3-carboxamide;
1-(4-cyanobenzyl)-N-{(1S)-1-[(4-hydroxypiperidin-1-yl)carbonyl]-2,2-dimethylpropyl}-1H-indazole-3-carboxamide;
ethyl 3-{[(N-{[1-(4-fluorobenzyl)-1H-indazol-3-yl]carbonyl}-3-methyl-L-valyl)amino]methyl}-1,2,4-oxadiazole-5-carboxylate;
ethyl 3-{[(N-{[1-(4-cyanobenzyl)-1H-indazol-3-yl]carbonyl}-3-methyl-L-valyl)amino]methyl}-1,2,4-oxadiazole-5-carboxylate;
N-{(1S)-1-[({[5-(aminocarbonyl)-1,2,4-oxadiazol-3-yl]methyl}amino)carbonyl]-2,2-dimethylpropyl}-1-(4-fluorobenzyl)-1H-indazole-3-carboxamide;
N-{(1S)-1-[({[5-(aminocarbonyl)-1,2,4-oxadiazol-3-yl]methyl}amino)carbonyl]-2,2-dimethylpropyl}-1-(4-cyanobenzyl)-1H-indazole-3-carboxamide;
N-[(1S)-2,2-dimethyl-1-({[(3-methyl-1,2,4-oxadiazol-5-yl)methyl]amino}carbonyl)propyl]-1-(4-fluorobenzyl)-1H-indazole-3-carboxamide;
1-(4-cyanobenzyl)-N-[(1S)-2,2-dimethyl-1-({[(3-methyl-1,2,4-oxadiazol-5-yl)methyl]amino}carbonyl)propyl]-1H-indazole-3-carboxamide;
N-[(1S)-2,2-dimethyl-1-{[(2-morpholin-4-ylethyl)amino]carbonyl}propyl]-1-(4-fluorobenzyl)-1H-indazole-3-carboxamide;
1-(4-fluorobenzyl)-N-[(1S)-1-({[2-(4-hydroxypiperidin-1-yl)ethyl]amino}carbonyl)-2,2-dimethylpropyl]-1H-indazole-3-carboxamide;
N-[(1S)-2,2-dimethyl-1-({[2-(4-methylpiperazin-1-yl)ethyl]amino}carbonyl)propyl]-1-(4-fluorobenzyl)-1H-indazole-3-carboxamide;
N-{(1S)-1-[({2-[5-(aminocarbonyl)-1,2,4-oxadiazol-3-yl]ethyl}amino)carbonyl]-2,2-dimethylpropyl}-1-(4-fluorobenzyl)-1H-indazole-3-carboxamide;
N-{(1S)-1-[({2-[5-(aminocarbonyl)-1,2,4-oxadiazol-3-yl]ethyl}amino)carbonyl]-2,2-dimethylpropyl}-1-(4-cyanobenzyl)-1H-indazole-3-carboxamide;
N-[(1S)-2,2-dimethyl-1-({[2-(3-methyl-1,2,4-oxadiazol-5-yl)ethyl]amino}carbonyl)propyl]-1-(4-fluorobenzyl)-1H-indazole-3-carboxamide;
N-[(1S)-2,2-dimethyl-1-({[2-(5-methyl-1,3,4-oxadiazol-2-yl)ethyl]amino}carbonyl)propyl]-1-(4-fluorobenzyl)-1H-indazole-3-carboxamide;
N-[(1S)-1-({[2-(5-cyclopropyl-1,3,4-oxadiazol-2-yl)ethyl]amino}carbonyl)-2,2-dimethylpropyl]-1-(4-fluorobenzyl)-1H-indazole-3-carboxamide;
1-(4-fluorobenzyl)-N-[(1S)-1-({[(4-hydroxytetrahydro-2H-pyran-4-yl)methyl]amino}carbonyl)-2,2-dimethylpropyl]-1H-indazole-3-carboxamide;
1-(4-cyanobenzyl)-N-[(1S)-1-({[(4-hydroxytetrahydro-2H-pyran-4-yl)methyl]amino}carbonyl)-2,2-dimethylpropyl]-1H-indazole-3-carboxamide;
1-(4-fluorobenzyl)-N-[(1S)-1-{[(3R)-3-hydroxypyrrolidin-1-yl]carbonyl}-2,2-dimethylpropyl]-1H-indazole-3-carboxamide;
1-(4-cyanobenzyl)-N-[(1S)-1-{[(3R)-3-hydroxypyrrolidin-1-yl]carbonyl}-2,2-dimethylpropyl]-1H-indazole-3-carboxamide;
1-(cyclohexylmethyl)-N-[(1S)-1-({[(1-hydroxycyclopropyl)methyl]amino}carbonyl)-2,2-dimethylpropyl]-1H-indazole-3-carboxamide;
1-(4-cyanobutyl)-N-[(1S)-1-({[(1-hydroxycyclopropyl)methyl]amino}carbonyl)-2,2-dimethylpropyl]-1H-indazole-3-carboxamide;
1-(cyclohexylmethyl-N-[(1S)-1-{[(3-hydroxyphenyl)amino]carbonyl}-2,2-dimethylpropyl]-1H-indazole-3-carboxamide;
1-(4-cyanobutyl)-N-[(1S)-1-{[(3-hydroxyphenyl)amino]carbonyl}-2,2-dimethylpropyl]-1H-indazole-3-carboxamide;
1-(cyclohexylmethyl)-N-[(1S)-1-({[(1-hydroxycyclopentyl)methyl]amino}carbonyl)-2,2-dimethylpropyl]-1H-indazole-3-carboxamide;
1-(4-cyanobutyl)-N-[(1S)-1-({[(1-hydroxycyclopentyl)methyl]amino}carbonyl)-2,2-dimethylpropyl]-1H-indazole-3-carboxamide;
1-(cyclohexylmethyl)-N-[(1S)-1-({[1-(hydroxymethyl)cyclopropyl]amino}carbonyl)-2,2-dimethylpropyl]-1H-indazole-3-carboxamide;
1-(4-fluorobenzyl)-N-[(1S)-1-({[(4-hydroxytetrahydro-2H-pyran-4-yl)methyl]amino}carbonyl)-2,2-dimethylpropyl]-1H-indazole-3-carboxamide;
N-[(1S)-1-{[3-(aminocarbonyl)piperidin-1-yl]carbonyl}-2,2-dimethylpropyl]-1-(cyclohexylmethyl)-1H-indazole-3-carboxamide;
N-[(1S)-1-{[3-(aminocarbonyl)piperidin-1-yl]carbonyl}-2,2-dimethylpropyl]-1-(4-cyanobutyl)-1H-indazole-3-carboxamide;
N-[(1S)-1-(aminocarbonyl)-2,2-dimethylpropyl]-1-(4-cyanobenzyl)-5-fluoro-1H-indazole-3-carboxamide;
1-[4-(aminocarbonyl)benzyl]-N-[(1S)-1-(aminocarbonyl)-2,2-dimethylpropyl]-5-fluoro-1H-indazole-3-carboxamide;
1-[4-(aminocarbonyl)benzyl]-5-fluoro-N-[(1S)-1-{[(2-hydroxyethyl)amino]carbonyl}-2,2-dimethylpropyl]-1H-indazole-3-carboxamide;
1-(4-cyanobenzyl)-5-fluoro-N-[(1S)-1-{[(2-hydroxyethyl)amino]carbonyl}-2,2-dimethylpropyl]-1H-indazole-3-carboxamide;
1-(4-cyanobenzyl)-N-{(1S)-1-[(cyclopropylamino)carbonyl]-2,2-dimethylpropyl}-5-fluoro-1H-indazole-3-carboxamide;
N-{[1-(4-cyanobenzyl)-5-fluoro-1H-indazol-3-yl]carbonyl}-3-methyl-L-valylglycinamide;
N-{[1-(4-cyanobenzyl)-5-fluoro-1H-indazol-3-yl]carbonyl}-3-methyl-L-valylglycine;
N-[(1S)-1-(aminocarbonyl)-2,2-dimethylpropyl]-5-fluoro-1-(4-fluorobenzyl)-1H-indazole-3-carboxamide;
N-{(1S)-1-[(cyclopropylamino)carbonyl]-2,2-dimethylpropyl}-5-fluoro-1-(4-fluorobenzyl)-1H-indazole-3-carboxamide;
5-fluoro-1-(4-fluorobenzyl)-N-[(1S)-1-{[(2-hydroxyethyl)amino]carbonyl}-2,2-dimethylpropyl]-1H-indazole-3-carboxamide;
N-{[5-fluoro-1-(4-fluorobenzyl)-1H-indazol-3-yl]carbonyl}-3-methyl-L-valylglycinamide;
5-fluoro-1-(4-fluorobenzyl)-N-[(1S)-1-{[(3-hydroxypropyl)amino]carbonyl}-2,2-dimethylpropyl]-1H-indazole-3-carboxamide;
N-[(1S)-1-(aminocarbonyl)-2,2-dimethylpropyl]-7-fluoro-1-(4-fluorobenzyl)-1H-indazole-3-carboxamide;
N-{(1S)-1-[(cyclopropylamino)carbonyl]-2,2-dimethylpropyl}-7-fluoro-1-(4-fluorobenzyl)-1H-indazole-3-carboxamide;
7-fluoro-1-(4-fluorobenzyl)-N-[(1S)-1-{[(2-hydroxyethyl)amino]carbonyl}-2,2-dimethylpropyl]-1H-indazole-3-carboxamide;
7-fluoro-1-(4-fluorobenzyl)-N-[(1S)-1-{[(3-hydroxypropyl)amino]carbonyl}-2,2-dimethylpropyl]-1H-indazole-3-carboxamide;
N-{[7-fluoro-1-(4-fluorobenzyl)-1H-indazol-3-yl]carbonyl}-3-methyl-L-valylglycinamide;
N-{(1S)-1-[({[5-(aminocarbonyl)-1,3,4-oxadiazol-2-yl]methyl}amino)carbonyl]-2,2-dimethylpropyl}-7-fluoro-1-(4-fluorobenzyl)-1H-indazole-3-carboxamide;
N-[(1S)-1-(aminocarbonyl)-2,2-dimethylpropyl]-7-chloro-1-(4-fluorobenzyl)-1H-indazole-3-carboxamide;
7-chloro-N-{(1S)-1-[(cyclopropylamino)carbonyl]-2,2-dimethylpropyl}-1-(4-fluorobenzyl)-1H-indazole-3-carboxamide;
7-chloro-1-(4-fluorobenzyl)-N-[(1S)-1-{[(2-hydroxyethyl)amino]carbonyl}-2,2-dimethylpropyl]-1H-indazole-3-carboxamide;
7-chloro-1-(4-fluorobenzyl)-N-[(1S)-1-{[(3-hydroxypropyl)amino]carbonyl}-2,2-dimethylpropyl]-1H-indazole-3-carboxamide;
N-{[7-chloro-1-(4-fluorobenzyl)-1H-indazol-3-yl]carbonyl}-3-methyl-L-valylglycinamide;
N-{(1S)-1-[({2-[(cyclopropylsulfonyl)amino]ethyl}amino)carbonyl]-2,2-dimethylpropyl}-7-fluoro-1-(4-fluorobenzyl)-1H-indazole-3-carboxamide;
7-chloro-N-{(1S)-1-[({2-[(cyclopropylsulfonyl)amino]ethyl}amino)carbonyl]-2,2-dimethylpropyl}-1-(4-fluorobenzyl)-1H-indazole-3-carboxamide;
N-{[7-fluoro-1-(4-fluorobenzyl)-1H-indazol-3-yl]carbonyl}-3-methyl-L-valylglycine;
N-{[7-fluoro-1-(4-fluorobenzyl)-1H-indazol-3-yl]carbonyl}-3-methyl-L-valyl-D-alanine;
N-{[7-chloro-1-(4-fluorobenzyl)-1H-indazol-3-yl]carbonyl}-3-methyl-L-valyl-D-alanine;
7-chloro-N-[(1S)-1-({[(2 S)-2,3-dihydroxypropyl]amino}carbonyl)-2,2-dimethylpropyl]-1-(4-fluorobenzyl)-1H-indazole-3-carboxamide;
N-[(1S)-1-({[(2S)-2,3-dihydroxypropyl]amino}carbonyl)-2,2-dimethylpropyl]-7-fluoro-1-(4-fluorobenzyl)-1H-indazole-3-carboxamide;
7-chloro-N-[(1S)-1-({[(2R)-2,3-dihydroxypropyl]amino}carbonyl)-2,2-dimethylpropyl]-1-(4-fluorobenzyl)-1H-indazole-3-carboxamide;
N-[(1S)-1-({[(2R)-2,3-dihydroxypropyl]amino}carbonyl)-2,2-dimethylpropyl]-7-fluoro-1-(4-fluorobenzyl)-1H-indazole-3-carboxamide;
N-{(1S)-1-[({[5-(aminocarbonyl)-1,3,4-oxadiazol-2-yl]methyl}amino)carbonyl]-2,2-dimethylpropyl}-7-chloro-1-(4-fluorobenzyl)-1H-indazole-3-carboxamide;
N-{[7-chloro-1-(4-fluorobenzyl)-1H-indazol-3-yl]carbonyl}-3-methyl-L-valylglycine;
N-[(1S)-1-(aminocarbonyl)-2,2-dimethylpropyl]-7-chloro-1-(4-cyanobenzyl)-1H-indazole-3-carboxamide;
7-chloro-1-(4-cyanobenzyl)-N-{(1S)-1-[(cyclopropylamino)carbonyl]-2,2-dimethylpropyl}-1H-indazole-3-carboxamide;
7-chloro-1-(4-cyanobenzyl)-N-[(1S)-1-{[(2-hydroxyethyl)amino]carbonyl}-2,2-dimethylpropyl]-1H-indazole-3-carboxamide;
7-chloro-1-(4-cyanobenzyl)-N-[(1S)-1-{[(3-hydroxypropyl)amino]carbonyl}-2,2-dimethylpropyl]-1H-indazole-3-carboxamide;
N-{[7-chloro-1-(4-cyanobenzyl)-1H-indazol-3-yl]carbonyl}-3-methyl-L-valylglycinamide;
7-chloro-1-(4-cyanobenzyl)-N-{(1S)-1-[({2-[(cyclopropylsulfonyl)amino]ethyl}amino)carbonyl]-2,2-dimethylpropyl}-1H-indazole-3-carboxamide;
7-chloro-1-(4-cyanobenzyl)-N-[(1S)-1-({[(2S)-2,3-dihydroxypropyl]amino}carbonyl)-2,2-dimethylpropyl]-1H-indazole-3-carboxamide;
7-chloro-1-(4-cyanobenzyl)-N-[(1S)-1-({[(2R)-2,3-dihydroxypropyl]amino}carbonyl)-2,2-dimethylpropyl]-1H-indazole-3-carboxamide;
N-{[7-chloro-1-(4-cyanobenzyl)-1H-indazol-3-yl]carbonyl}-3-methyl-L-valylglycine;
N-{[7-chloro-1-(4-cyanobenzyl)-1H-indazol-3-yl]carbonyl}-3-methyl-L-valyl-D-alanine;
N-{[1-(3-fluorobenzyl)-1H-indazol-3-yl]carbonyl}-3-methyl-L-valylglycine;
N-{[1-(2-fluorobenzyl)-1H-indazol-3-yl]carbonyl}-3-methyl-L-valylglycine;
N-{[1-(2,4-difluorobenzyl)-1H-indazol-3-yl]carbonyl}-3-methyl-L-valylglycine; and
N-{[1-(3,4-difluorobenzyl)-1H-indazol-3-yl]carbonyl}-3-methyl-L-valylglycine.

14. A compound, or a pharmaceutically acceptable salt thereof, selected from the group consisting of
N-[(1S)-1-(aminocarbonyl)-2,2-dimethylpropyl]-1-(2-fluorobenzyl)-1H-pyrazolo[3,4-b]pyridine-3-carboxamide;
N-[(1S,2R)-1-(aminocarbonyl)-2-hydroxypropyl]-1-(2-fluorobenzyl)-1H-pyrazolo[3,4-b]pyridine-3-carboxamide;
N-[(1S)-1-(aminocarbonyl)-3-methylbutyl]-1-(2-fluorobenzyl)-1H-pyrazolo[3,4-b]pyridine-3-carboxamide;
1-(2-fluorobenzyl)-N-[(1S)-1-(hydroxymethyl)-2,2-dimethylpropyl]-1H-pyrazolo[3,4-b]pyridine-3-carboxamide;
N-[(1S)-1-(aminocarbonyl)-2,2-dimethylpropyl]-1-(pyridin-2-ylmethyl)-1H-pyrazolo[3,4-b]pyridine-3-carboxamide;
N-[(1S)-1-(aminocarbonyl)-2-methylpropyl]-1-(pyridin-2-ylmethyl)-1H-pyrazolo[3,4-b]pyridine-3-carboxamide;
N-[(1S)-1-(aminocarbonyl)-3-methylbutyl]-1-(pyridin-2-ylmethyl)-1H-pyrazolo[3,4-b]pyridine-3-carboxamide;
N-[(1-benzyl-1H-pyrazolo[3,4-b]pyridin-3-yl)carbonyl]-3-methyl-L-valine;
N-[(1S)-1-(aminocarbonyl)-2,2-dimethylpropyl]-1-benzyl-1H-pyrazolo[3,4-b]pyridine-3-carboxamide;
N-[(1S)-1-(aminocarbonyl)-2-methylpropyl]-1-benzyl-1H-pyrazolo[3,4-b]pyridine-3-carboxamide;
1-benzyl-N-[(1S)-1-(hydroxymethyl)-2,2-dimethylpropyl]-1H-pyrazolo[3,4-b]pyridine-3-carboxamide;
N-[(1S)-1-(aminocarbonyl)-3-methylbutyl]-1-benzyl-1H-pyrazolo[3,4-h]pyridine-3-carboxamide;
N-[(1S,2R)-1-(aminocarbonyl)-2-hydroxypropyl]-1-benzyl-1H-pyrazolo[3,4-b]pyridine-3-carboxamide;
N-[(1S)-1-(hydroxymethyl)-2,2-dimethylpropyl]-1-(pyridin-2-ylmethyl)-1H-pyrazolo[3,4-b]pyridine-3-carboxamide;
N-[(1S,2R)-1-(aminocarbonyl)-2-hydroxypropyl]-1-(pyridin-2-ylmethyl)-1H-pyrazolo[3,4-b]pyridine-3-carboxamide; and
N-[(1S)-1-(aminocarbonyl)-2-methylpropyl]-1-(2-fluorobenzyl)-1H-pyrazolo[3,4-b]pyridine-3-carboxamide.

15. A compound according to claim 1 of the general formula 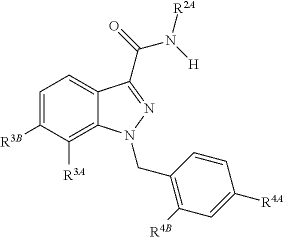
or a pharmaceutically acceptable salt thereof wherein
R^{2A} is selected from
NR¹¹R¹²—C(O)—R¹³CH—,
C₁-C₆ alkoxy-C(O)—(CH₂), —NR¹⁵—C(O)—R¹³CH—,
NR¹⁷R¹⁸—C(O)—(CH₂), NR¹⁹—C(O)—R¹³CH—,
R²⁴₁₋₅-heteroaryl-NR¹⁵—C(O)—R¹³CH—,
NR²⁷R²⁸—(CH₂), —NR²⁹—C(O)—R¹³CH—,
R³⁰—SO₂—NR³¹—(CH₂)ₙ—NR¹⁵—C(O)—R¹³CH—,
R³⁰—SO₂—(CH₂)ₙ—NR³¹—C(O)—R¹³CH—,
R³²—C(O)—R³³CH—NR³⁴—C(O)—R¹³CH—,
R³²—C(O)—(CH₂)ₙ—NR³⁴—C(O)—R¹³CH—,
R³⁵₁₋₅-heteroaryl-(CH₂)ₙ—NR³⁶—C(O)—R¹³CH—,
R³⁷₁₋₅-heterocyclyl-(C₂)ₙ—NR³⁶—C(O)—R¹³CH—,
R³⁷₁₋₅-heterocyclyl-C(O)—R¹³CH—,
R³⁸₁₋₅-aryl-R³⁹C—NR⁴⁰—C(O)—R¹³CH—, or
R³⁸₁₋₅-aryl-(CH₂)ₙ—NR⁴⁰—C(O)—R¹³CH—
wherein
R¹¹ and R¹² are independently H, C₁-C₆ alkyl, OH—C₁-C₆ alkyl, (OH)₂—C₁-C₆ alkyl, C₁-C₆ alkoxy-(CH₂)ₙ—, C₃-C₇ cycloalkyl, cyano-C₁-C₆ alkyl, (OH—C₁-C₆ alkyl)₂-C₁-C₆ alkylene, OH—C₃-C₇ cycloalkyl-(CH₂)ₙ—, OH—(CH₂)ₙ—C₃-C₇ cycloalkyl-, or OH-aryl;
R¹³ is H, C₁-C₆ alkyl, OH—C₁-C₆ alkyl, aryl, aryl-(CH₂)ₙ—, or C₃-C₇ cycloalkyl;
R¹⁵, R²⁹, R³¹, R³³, R³⁴, R³⁶, R³⁹ and R⁴⁰ are independently H or C₁-C₆ alkyl;
R¹⁷, R¹⁸ and R¹⁹ are independently H or C₁-C₆ alkyl;
each R²⁴ is independently H, C₁-C₆ alkyl, C₃-C₇ cycloalkyl, C₁-C₆ haloalkyl, oxo, NH₂, C₁-C₆ alkoxy-C(O)—, NH₂—C(O)—(CH₂)ₙ—, NH₂—C(O)—, NH₂—C(O)—NH—, OH—C(O)—, NH₂—C(O)—(CH₂)ₙ—NH—C(O)—, (OH)₂—C₁-C₆ alkyl-NH—C(O)—, or OH—C₁-C₆ alkyl-NH—C(O)—;
each R²⁵ is independently H or oxo;
R²⁷ and R²⁸ independently are H, NH₂—C(O)—, or C₃-C₇ cycloalkyl-C(O)—;
R³⁰ is C₁-C₆ alkyl, C₃-C₇ cycloalkyl or NH₂;
R³² is OH;
R³⁵ is independently H, C₁-C₆ alkyl, NH₂—C(O)—, C₁-C₆ alkoxy-C(O)— or C₃-C₇ cycloalkyl;
each R³⁷ is independently H, NH₂C(O)— or OH;
each R³⁸ is independently H, NH₂SO₂—, cyano, heteroaryl, OH, halo, C₁-C₆ alkoxy, OH—C(O)—, or C₁-C₆ alkoxy-C(O)—;
n is an integer from 1 to 6;
R^{3A} and R^{3B} are independently selected from H and halo;
R^{4A} is selected from F and CN; and
R^{4B} is selected from H and F.

16. A compound according to claim 15 wherein R¹³ is C₁-C₆ alkyl.

17. A compound according to claim 16 wherein R¹³ is branched C₃-C₆ alkyl.

18. A compound according to claim 17 wherein R¹³ is tert-butyl

19. A compound according to claim 1 of the general formula 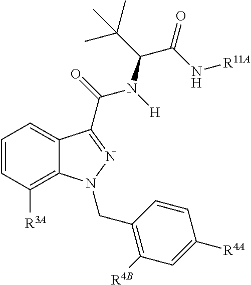
or a pharmaceutically acceptable salt thereof wherein
R^{3A} is selected from H, F and Cl;
R^{4A} is selected from F and CN;
R^{4B} is selected from H and F; and
R^{11A} is selected from H, OH—C₁-C₆ alkyl and (OH)₂—C₁-C₆ alkyl.

20. A compound according to claim 1 of the general formula 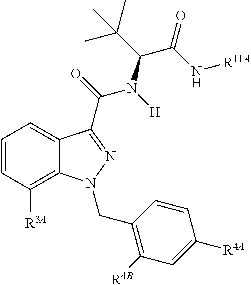
or a pharmaceutically acceptable salt thereof wherein
R^{3A} is selected from H, F and Cl;
R^{4A} is selected from F and CN;
R^{4B} is selected from H and F; and
R^{11A} is selected from H, 2-hydroxyethyl and 2,3-dihydroxypropyl.

21. A pharmaceutical composition comprising a compound of Formula I according to claim 1 or a pharmaceutically acceptable salt, enantiomer, or racemate thereof.

22. A compound according to claim 1 a pharmaceutically acceptable salt, enantiomer, or racemate thereof, for use as a medicament.

23. A compound according to claim 22 for use in treatment of a CB1 mediated disorder.

24. A compound according to claim 23 for use in treatment of pain.

25. Use of a compound according to claim 1 or a pharmaceutically acceptable salt, enantiomer, or racemate thereof, for the manufacture of a medicament for the treatment of a CB1 mediated disorder.

26. The use according to claim 25 wherein the CB1 mediated disorder is pain.

27. Use of a compound according to claim 1 or a pharmaceutically acceptable salt, enantiomer, or racemate thereof, for the treatment of a CB1 mediated disorder.

28. The use according to claim 27 wherein the CB1 mediated disorder is pain.

29. A method for the treatment of a CB1 mediated disorder in a subject in need of such treatment or prevention, wherein the method comprises administering to the subject an amount of a compound of Formula I according to claim 1 or a pharmaceutically acceptable salt, enantiomer, or racemate thereof, wherein the amount of the compound is effective for the treatment or prevention of the CB1 mediated disorder.

30. The method of claim 29 wherein the CB1 mediated disorder is pain.
